(19) United States
(12) Patent Application Publication
(10) Pub. No.: US 2023/0062179 A1
(43) Pub. Date: Mar. 2, 2023

(54) METHODS FOR GENERATING NEW GENES IN ORGANISM AND USE THEREOF

(71) Applicant: Qingdao Kingagroot Chemical Compound Co., Ltd., Qingdao (CN)

(72) Inventors: LINJIAN JIANG, Qingdao (CN); JIYAO WANG, Qingdao (CN); SUDONG MO, Qingdao (CN); BO CHEN, Qingdao (CN); QIANG HU, Qingdao (CN); DEHUI DING, Qingdao (CN); HUARONG LI, Qingdao (CN)

(21) Appl. No.: 17/663,075

(22) Filed: May 12, 2022

Related U.S. Application Data
(63) Continuation-in-part of application No. 17/246,971, filed on May 3, 2021, now abandoned.
Application No. 17/246,971 is a continuation-in-part of application No. 17/264,367, filed on Jan. 29, 2021.

(30) Foreign Application Priority Data
Nov. 6, 2019 (CN) 201911073406.1
Oct. 30, 2020 (CN) 202011190279.6

(51) Int. Cl. C12N 15/82; C07K 14/415; A01H 5/00; A01H 1/00

(52) U.S. Cl. CPC C12N 15/8216; C07K 14/415; C12N 15/8274; A01H 5/00; A01H 1/00; C12N 2310/20

(57) ABSTRACT

The present invention relates to the technical fields of genetic engineering and bioinformatics, in particular, to a method for creating a new gene in an organism in the absence of an artificial DNA template, and a use thereof. The method comprises simultaneously generating DNA breaks at two or more different specific sites in the organism's genome, wherein the specific sites are genomic sites capable of separating different gene elements or different protein domains, and the DNA breaks are ligated to each other through non-homologous end joining (NHEJ) or homologous repair to generate a new combination of the different gene elements or different protein domains that is different from the original genome sequence, thereby creating a new gene. The new gene of the invention can change the growth, development, resistance, yield and other traits of the organism, and has great value in application.

### TECHNICAL FIELD

The present invention relates to the technical fields of genetic engineering and bioinformatics, and in particular, a method for creating a new gene in an organism in the absence of an artificial DNA template, and use thereof.

### BACKGROUND ART

Generally speaking, a complete gene expression cassette in an organism comprises a promoter, 5′ untranslated region (5′ UTR), coding region (CDS) or non-coding RNA region (Non-coding RNA), 3′ untranslated region (3′UTR), a terminator and many other elements. Non-coding RNA can perform its biological functions at the RNA level, including rRNA, tRNA, snRNA, snoRNA and microRNA. The CDS region contains exons and introns. After the transcribed RNA is translated into a protein, the amino acids of different segments usually form different domains. The specific domains determine the intracellular localization and function of the protein (such as nuclear localization signal, chloroplast leading peptide, mitochondrial leading peptide, DNA binding domain, transcription activation domain, enzyme catalytic center, etc.). For non-coding RNA, different segments also have different functions. When one or several elements of a gene change, a new gene will be formed, which may have new functions. For example, an inversion event of a 1.7 Mb chromosome fragment occurred upstream of the PpOFP1 gene of flat peach may result in a new promoter, which will significantly increase the expression of PpOFP1 in peach fruit with flat shape in the S2 stage of fruit development as compared to that in peach fruit with round shape, thereby inhibit the vertical development of peach fruit and result in the flat shape phenotype in flat peach (Zhou et al. 2018. A 1.7-Mb chromosomal inversion downstream of a PpOFP1 gene is responsible for flat fruit shape in peach. Plant Biotechnol. J. DOI: 10.1111/pbi.13455).

The natural generation of new genes in biological genomes requires a long evolutionary process. According to the research work, the molecular mechanisms for the generation of new genes include exon rearrangement, gene duplication, retrotransposition, and integration of movable elements (transposons, retrotransposons), horizontal gene transfer, gene fusion splitting, de novo origination, and many other mechanisms, and new genes may be retained in species under the action of natural selection through the derivation and functional evolution. The relatively young new genes that have been identified in fruit flies, Arabidopsis thaliana, and primates have a history of hundreds of thousands to millions of years according to a calculation (Long et al. 2012. The origin and evolution of new genes. Methods Mol Biol. DOI: 10.1007/978-1-61779-585-5_7). Therefore, in the field of genetic engineering and biological breeding, taking plants as an example, if it is desired to introduce a new gene into a plant (even if all the gene elements of the new gene are derived from different genes of the species itself), it can only be achieved through the transgenic technology. That is, the elements from different genes are assembled together in vitro to form a new gene, which is then transferred into the plant through transgenic technology. It is characterized in that the assembly of new gene needs to be carried out in vitro, resulting in transgenic crops.

The gene editing tools represented by CRISPR/Cas9 and the like can efficiently and accurately generate double-strand breaks (DSB) at specific sites in the genome of an organism, and then the double-strand breaks (DSB) are repaired through the cell's own non-homologous end repair or homologous recombination mechanisms, thereby generating site-specific mutations. The current applications of the gene editing technique mainly focus on the editing of the internal elements of a single gene, mostly the editing of a CDS exon region. Editing an exon usually results in frameshift mutations in the gene, leading to the function loss of the gene. For this reason, the gene editing tools such as CRISPR/Cas9 are also known as gene knockout (i.e., gene destruction) tools. In addition to the CDS region, the promoter, 5′UTR and other regions can also be knocked out to affect the expression level of a gene. These methods all mutate existing genes without generating new genes, so it is difficult to meet some needs in production. For example, for most genes, the existing gene editing technology is difficult to achieve the up-regulation of gene expression, and it is also difficult to change the subcellular localization of a protein or change the functional domain of protein. There are also reports in the literature of inserting a promoter or enhancer sequence upstream of an existing gene to change the expression pattern of the gene so as to produce new traits (Lu et al. 2020. Targeted, efficient sequence insertion and replacement in rice. Nat Biotechnol. DOI: 10.1038/s41587-020-0581-5), but this method requires the provision of foreign DNA templates, so strict regulatory procedures similar to genetically modified crops apply, and the application is restricted.

### SUMMARY OF THE INVENTION

In order to solve the above-mentioned problems in the prior art, the present invention provides a method for creating a new gene in an organism in the absence of an artificial DNA template by simultaneously generating two or more DNA double-strand breaks at a combination of specific sites in the organism's genome, and use thereof.

In one aspect, the present invention provides a method for creating a new gene in an organism, comprising the following steps:

simultaneously generating DNA breaks at two or more different specific sites in the organism's genome, wherein the specific sites are genomic sites capable of separating different genetic elements or different protein domains, ligating the DNA breaks to each other by a non-homologous end joining (NHEJ) or homologous repair, generating a new combination of the different genetic elements or different protein domains that is different from the original genome sequence, thereby creating the new gene.

In another aspect, the present invention provides a method for in vivo creation of new genes that can be stably inherited in an organism, characterized by comprising the following steps:

(1) simultaneously generating double-stranded DNA breaks at two or more different specific sites in the organism's genome, wherein the specific sites are capable of separating different gene elements or different protein domains, and the DNA breaks are then ligated to each other by a non-homologous end joining (NHEJ) or homologous repair, generating a new combination or assemble of the different gene elements or different protein domains derived from the original genomic sequence, thereby the new gene is generated;

in a specific embodiment, it also includes (2) designing primer pairs that can specifically detect the above-mentioned new combination or assemble, then cells or tissues containing the new genes can be screened out by PCR test, and the characteristic sequences of new combinations of gene elements can be determined by sequencing; and

(3) cultivating the above-screened cells or tissues to obtain T0 generation organisms, and perform PCR tests and sequencing on the organisms for two consecutive generations including the T0 generation and its bred T1 or at least three consecutive generations to select the organisms containing the above-mentioned characteristic sequence of new combination of gene elements, namely, a new gene that can be stably inherited has been created in the organism;

optionally, it also includes (4) testing the biological traits or phenotypes related to the function of the new gene, to determine the genotype that can bring beneficial traits to the organism, and to obtain a new functional gene that can be stably inherited.

In a specific embodiment, in the step (1), DNA breaks are simultaneously generated at two different specific sites in the genome of the organism, wherein one site is the genomic locus between the promoter region and the coding region of a gene, meanwhile, the other site is between the promoter region and the coding region of another gene with different expression patterns, resulting in a new combination of the promoter of one gene and the coding region of the other gene that has a different expression pattern; preferably, a combination of the strong promoter and the gene of interest is eventually produced.

In another specific embodiment, in the step (1), DNA breaks are simultaneously generated at three different specific sites in the genome of the organism, the three specific sites include two genomic sites whose combination capable of cutting off the promoter region of a highly expressed gene and the third genomic site between the coding region and the promoter region of the gene of interest that has a different expression pattern; or a genomic site between the promoter region and the coding region of a highly expressed gene and another two genomic sites whose combination capable of cutting off the coding region fragment of the gene of interest that has a different expression pattern; then through gene editing at the above-mentioned sites, translocation editing events can be generated, in which the strong promoter fragment that is inserted upstream of the coding region of the gene of interest, or the coding region fragment of the gene of interest is inserted the downstream of the promoter of another highly expressed gene, finally, the combination of the promoter of one gene and the coding region of the other gene of interest with different expression patterns is generated.

In a specific embodiment, the “two or more different specific sites” may be located on the same chromosome or on different chromosomes. When they locate on the same chromosome, the chromosome fragment resulting from the DNA breaks simultaneously occurring at two specific sites may be deleted, inversed or replicating doubled after repair; when they locate on different chromosomes, the DNA breaks generated at two specific sites may be ligated to each other after repair to produce a crossover event of the chromosome arms. These events can be identified and screened by PCR sequencing with specifically designed primers.

In a specific embodiment, the “two or more different specific sites” may be specific sites on at least two different genes, or may be at least two different specific sites on the same gene.

In a specific embodiment, the transcription directions of the “at least two different genes” may be the same or different (opposite or toward each other).

The “gene elements” comprise a promoter, a 5′ untranslated region (5′UTR), a coding region (CDS) or non-coding RNA region (Non-coding RNA), a 3′ untranslated region (3′UTR) and a terminator of the gene.

In a specific embodiment, the combination of different gene elements refers to a combination of the promoter of one of the two genes with different expression patterns and the CDS or non-coding RNA region of the other gene.

In another specific embodiment, the combination of different gene elements refers to a combination of a region from the promoter to the 5′UTR of one of two genes with different expression patterns and the CDS or non-coding RNA region of the other gene.

In a specific embodiment, the “different expression patterns” refer to different levels of gene expression.

In another specific embodiment, the “different expression patterns” refer to different tissue-specific of gene expression.

In another specific embodiment, the “different expression patterns” refer to different developmental stage-specificities of gene expression.

In another specific embodiment, the combination of different gene elements is a combination of adjacent gene elements within the same gene.

The “protein domains” refer to a DNA fragment corresponding to a specific functional domain of a protein; it includes but is not limited to nuclear localization signal, chloroplast leading peptide, mitochondrial leading peptide, phosphorylation site, methylation site, transmembrane domain, DNA binding domain, transcription activation domain, receptor activation domain, enzyme catalytic center, etc.

In a specific embodiment, the combination of different protein domains refers to a combination of a localization signal region of one of two protein coding genes with different subcellular localizations and a mature protein coding region of the other gene.

In a specific embodiment, the “different subcellular locations” include, but are not limited to, a nuclear location, a cytoplasmic location, a cell membrane location, a chloroplast location, a mitochondrial location, or an endoplasmic reticulum membrane location.

In another specific embodiment, the combination of different protein domains refers to a combination of two protein domains with different biological functions.

In a specific embodiment, the “different biological functions” include, but are not limited to, recognition of specific DNA or RNA conserved sequence, activation of gene expression, binding to protein ligand, binding to small molecule signal, ion binding, or specific enzymatic reaction.

In another specific embodiment, the combination of different protein domains refers to a combination of adjacent protein domains in the same gene.

In another specific embodiment, the combination of gene elements and protein domains refers to a combination of protein domains and adjacent promoters, 5′UTR, 3′UTR or terminators in the same gene.

Specifically, the exchange of promoters of different genes can be achieved by inversion of chromosome fragments: when two genes located on the same chromosome have different directions, DNA breaks can be generated at specific sites between the promoter and CDS of each of the two genes, the region between the breaks can be inverted, thereby the promoters of these two genes would be exchanged, and two new genes would be generated at both ends of the inverted chromosome segment. The different directions of the two genes may be that their 5′ ends are internal, namely both genes are in opposite directions, or their 5′ ends are external, namely both genes are towards each other. Where the genes are in opposite directions, the promoters of the genes would be inverted, as shown in Scheme 1 of FIG. 2; where the genes are towards each other, the CDS regions of the genes would be inverted, as shown in Scheme 1 of FIG. 4. The inverted region can be as short as less than 10 kb in length, with no other genes therebetween; or the inverted region can be very long, reaching up to 300 kb-3 Mb, and containing hundreds of genes.

It is also possible to create a new gene by doubling a chromosome fragment: where two genes located on the same chromosome are in the same direction, DNA breaks can be generated in specific sites between the promoter and CDS of each of the two genes, the region between the breaks can be doubled by duplication, and a new gene would be created at the junction of the doubled segment by fusing the promoter of the downstream gene to the CDS region of the upstream gene, as shown in FIG. 1 Scheme 1 and FIG. 3. The length of the doubled region can be in the range of 500 bp to 5 Mb, which can be very short with no other genes therebetween, or can be very long to contain hundreds of genes. Although this method will induce point mutations in the regions between the promoters and the CDS region of the original two genes, such small-scale point mutations generally have little effect on the properties of the gene expression, while the new genes created by promoter replacement will have new properties of expression. Or alternatively, DNA breaks can be generated at specific positions on both sides of a protein domain of a same gene, and the region between the breaks can be doubled by duplication, thereby creating a new gene with doubled specific functional domains.

The present invention also provides a new gene obtainable by the present method.

Compared with the original genes, the new gene may have different promoter and therefore have expression characteristics in terms of tissues or intensities or developmental stages, or have new amino acid sequences.

The “new amino acid sequence” can either be a fusion of the whole or partial coding regions of two or more gene, or a doubling of a partial protein coding region of the same gene.

The present invention further provides use of the gene in conferring or improving a resistance/tolerance trait or growth advantage trait in an organism.

In a specific embodiment, in the combination of different gene elements, one element is a plant endogenous strong promoter or the region from a strong promoter to 5′UTR, and the other is the HPPD, EPSPS, PPO, ALS, ACCase, GS, PDS, DHPS, DXPS, HST, SPS, cellulose synthesis, VLCFAS, fatty acid thioesterase, serine threonine protein phosphatase or lycopene cyclase gene coding region of the same plant.

In a specific embodiment, the present invention also provides a new gene obtainable by the present method, the level of the new gene expression is up-regulated relative to the plant endogenous wild-type HPPD, EPSPS, PPO, ALS, ACCase, GS, PDS, DHPS, DXPS, HST, SPS, cellulose synthesis, VLCFAS, fatty acid thioesterase, serine threonine protein phosphatase or lycopene cyclase gene.

In a specific embodiment, the present invention also provides use of the new gene in the improvement of the resistance or tolerance to a corresponding inhibition of HPPD, inhibition of EPSPS, inhibition of PPO, inhibition of ALS, inhibition of ACCase, inhibition of GS, inhibition of PDS, inhibition of DHPS, inhibition of DXPS, inhibition of HST, inhibition of SPS, inhibition of cellulose synthesis, inhibition of VLCFAS, inhibition of fatty acid thioesterase, inhibition of serine threonine protein phosphatase or inhibition of lycopene cyclase herbicide in a plant cell, a plant tissue, a plant part or a plant.

In another specific embodiment, in the combination of different gene elements, one element is an endogenous strong promoter or the region from a strong promoter to 5′UTR of the organism, and the other is a gene coding region of any one of the P450 family in the same organism.

In another specific embodiment, the present invention also provides a new gene obtainable by the present method, the level of the new gene expression is up-regulated relative to the corresponding endogenous wild-type P450 gene of the organism.

In another specific embodiment, the present invention also provides use of the new gene in enhancing biological detoxification capability, stress tolerance or secondary metabolic ability.

In another specific embodiment, the said P450 gene is rice OsCYP81A gene or maize ZmCYP81A9 gene.

In another specific embodiment, the present invention also provides a new gene obtainable by the present method, the level of the new gene expression is up-regulated relative to the rice endogenous OsCYP81A6 gene or maize endogenous ZmCYP81A9 gene, respectively.

In another specific embodiment, the present invention also provides use of the new gene in the improvement of the resistance or tolerance of rice or maize to a herbicide.

In another specific embodiment, in the combination of different gene elements, one element is a maize endogenous strong promoter or the region from a strong promoter to 5′UTR, and the other is the coding region of maize gene ZMM28 (Zm00001d022088), ZmKNR6 or ZmBAMld.

In another specific embodiment, the present invention also provides a new gene obtainable by the present method, the level of the new gene expression is up-regulated relative to the plant endogenous wild-type ZMM28 gene, ZmKNR6 gene or ZmBAMld gene, respectively.

In another specific embodiment, the present invention also provides use of the new gene in the improvement of maize yield.

In another specific embodiment, in the combination of different gene elements, one element is a rice endogenous strong promoter or the region from a strong promoter to 5′UTR, and the other is the coding region of rice gene COLD1 or OsCPK24.

In another specific embodiment, the present invention also provides a new gene obtainable by the present method, the level of the new gene expression is up-regulated relative to the rice endogenous wild-type COLD1 or OsCPK24 gene, respectively.

In another specific embodiment, the present invention also provides use of the new gene in the improvement of cold tolerance in rice.

In another specific embodiment, in the combination of different gene elements, one element is an endogenous strong promoter or the region from a strong promoter to 5′UTR of the organism, and the other is a gene coding region of any one of the ATP-binding cassette (ABC) transporter family in the same organism.

In another specific embodiment, the present invention also provides a new gene obtainable by the present method, the level of the new gene expression is up-regulated relative to the corresponding endogenous wild-type ATP-binding cassette (ABC) transporter gene of the organism.

In another specific embodiment, the present invention also provides use of the new gene in enhancing biological detoxification capability or stress tolerance.

In another specific embodiment, in the combination of different gene elements, one element is a plant endogenous strong promoter or the region from a strong promoter to 5′UTR of the plant, and the other is a gene coding region of any one of the NAC transcription factor family in the same plant.

In another specific embodiment, the said NAC transcription factor family gene is OsNAC045, OsNAC67, ZmSNAC1, OsNAC006, OsNAC42, OsSNAC1 or OsSNAC2.

In another specific embodiment, the present invention also provides a new gene obtainable by the present method, the level of the new gene expression is up-regulated relative to the corresponding plant endogenous wild-type NAC transcription factor family gene.

In another specific embodiment, the present invention also provides use of the new gene in enhancing plant stress tolerance or plant yield.

In another specific embodiment, in the combination of different gene elements, one element is a plant endogenous strong promoter or the region from a strong promoter to 5′UTR, and the other is the gene coding region of any one of MYB, MADS, DREB and bZIP transcription factor family in the same plant.

In another specific embodiment, the present invention also provides a new gene obtainable by the present method, the level of the new gene expression is up-regulated relative to the corresponding plant endogenous wild-type MYB transcription factor gene, MADS transcription factor family gene, DREB transcription factor family gene coding region or bZIP transcription factor family gene, respectively.

In another specific embodiment, the present invention also provides the use of new gene in enhancing plant stress tolerance or regulating plant growth and development.

In another specific embodiment, in the combination of different gene elements, one element is the promoter of any one of overexpression or tissue-specific expression rice genes listed in Table A, and the other is the protein coding region or the non-coding RNA region of another gene that is different from the selected promoter corresponding to the rice gene.

In another specific embodiment, the present invention also provides a new gene obtainable by the present method, the expression pattern of the new gene is changed relative to the selected protein coding region or the non-coding RNA region of the rice endogenous gene.

In another specific embodiment, the present invention also provides the use of new gene in regulating the growth and development of rice.

In another specific embodiment, in the combination of different gene elements, one element is a protein coding region or non-coding RNA region selected from any one of the biological functional genes listed in Table B to K, and the other is the promoter region of another gene that is different from the selected functional gene of the biological genome corresponding to the selected gene.

In another specific embodiment, the present invention also provides a new gene obtainable by the present method, the expression pattern of the new gene is changed relative to the selected functional gene.

In another specific embodiment, the present invention also provides use of the new gene in regulating the growth and development of organism.

In another specific embodiment, in the combination of different gene elements, one element is an endogenous strong promoter or the region from a strong promoter to 5′UTR of the organism, and the other is a gene coding region of any one of the GST (glutathione-s-transferases) family in the same organism.

In another specific embodiment, the present invention also provides a new gene obtainable by the present method, the level of the new gene expression is up-regulated relative to the corresponding endogenous GST (glutathione-s-transferases) family gene of the organism.

In another specific embodiment, the present invention also provides use of the new gene in enhancing biological detoxification capability or stress tolerance.

In another specific embodiment, the said GST family gene is wheat GST Cla47 (AY064480.1) gene, wheat GST 19E50 (AY064481.1), wheat GST28E45 (AY479764.1), maize ZmGSTIV, maize ZmGST6, maize ZmGST31, maize GSTI, maize GSTIII, maize GSTIV, maize GST5 or maize GST7 gene.

In another specific embodiment, the present invention also provides a new gene obtainable by the present method, the level of the new gene expression is up-regulated relative to the endogenous wheat GST Cla47 (AY064480.1) gene, wheat GST 19E50 (AY064481.1), wheat GST28E45 (AY479764.1), maize ZmGSTIV, maize ZmGST6, maize ZmGST31, maize GSTI, maize GSTIII, maize GSTIV, maize GST5 or maize GST7 gene, respectively.

In another specific embodiment, the present invention also provides use of the new gene in the improvement of the resistance or tolerance of wheat or maize to a herbicide.

In another specific embodiment, in the combination of different gene elements, one element is a rice endogenous strong promoter or the region from a strong promoter to 5′UTR, and the other is the coding region of any one of gene protein in rice GIF1 (Os04g0413500), NOG1 (OsO1g075220), LAIR (Os02g0154100), OSA1 (Os03g0689300), OsNRT1.1A (Os08g0155400), OsNRT2.3B (OsO1g0704100), OsRac1 (OsO1g0229400), OsNRT2.1 (Os02g0112100), OsGIF1 (Os03g0733600), OsNAC9 (Os03g0815100), CPB1/D11/GNS4 (Os04g0469800), miR1432 (Os04g0436100), OsNLP4 (Os09g0549450), RAG2 (Os07g0214300), LRKI1 (Os02g0154200), OsNHX1 (Os07t0666900), GW6 (Os06g0623700), WG7 (Os07g0669800), D11/OsBZR1 (Os04g0469800, Os07g0580500), OsAAP6 (Os07g0134000), OsLSK1 (Os01g0669100), IPA1 (Os08g0509600), SMG11 (Os01g0197100), CYP72A31 (Os01g0602200), SNAC1 (Os03g0815100), ZBED (Os01g0547200), OsSta2 (Os02g0655200), OsASR5 (Os11g0167800), OsCPK4 (Os02g03410), OsDjA9 (Os06g0116800), EUI (Os05g0482400), JMJ705 (Os01g67970), WRKY45 (Os05t0322900), OsRSR1 (Os05g0121600), OsRLCK5 (OsO1g0114100), APIP4 (OsO1g0124200), OsPAL6 (Os04t0518400), OsPAL8 (Os11g0708900), TPS46 (Os08t0168000), OsERF3 (Os01g58420) and OsYSL15 (Os02g0650300).

In another specific embodiment, the present invention also provides a new gene obtainable by the present method, the level of the new gene expression is up-regulated relative to the corresponding endogenous gene.

In another specific embodiment, the present invention also provides use of the new gene in rice breeding.

In another specific embodiment, in the combination of different gene elements, one element is a fish endogenous strong promoter, and the other is a gene coding region of GH1 (growth hormone 1) in the selected fish.

In another specific embodiment, the present invention also provides a fish endogenous high expression GH1 gene obtainable by the method.

In another specific embodiment, the present invention also provides use of the fish endogenous high expression GH1 gene in fish breeding.

In another specific embodiment, in the combination of different protein domains, one element is a wheat endogenous protein chloroplast localization signal domain, and the other is a wheat mature protein coding region of cytoplasmic localization phosphoglucose isomerase (PGIc).

In another specific embodiment, the present invention also provides a new gene obtainable by the present method, the new gene locates the phosphoglucose isomerase gene relative to the coding cytoplasm and its mature protein is located in the chloroplast.

In another specific embodiment, the present invention also provides use of the new gene in the improvement of wheat yield.

In another specific embodiment, in the combination of different protein domains, one element is a rice protein chloroplast localization signal domain (CTP), and the other is the mature protein coding region of OsGLO3, OsOXO3 or OsCATC.

In another specific embodiment, the present invention also provides a new gene obtainable by the present method, the mature protein of the new gene is located in chloroplast different from OsGLO3, OsOXO3 or OsCATC.

In another specific embodiment, the present invention also provides use of the new gene in improving the photosynthetic efficiency of rice.

In another specific embodiment, the present invention also provides a chloroplast localized protein OsCACT, the nucleotide encoding the protein has a sequence selected from the group consisting of:

(1) the nucleic acid sequence as shown in SEQ ID NO: 28, or a portion thereof or a complementary sequence thereof;

(2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

In another specific embodiment, the present invention also provides a chloroplast localized protein OsGLO3, the nucleotide encoding the protein has a sequence selected from the group consisting of:

(1) the nucleic acid sequence as shown in SEQ ID NO: 29, or a portion thereof or a complementary sequence thereof;

(2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

In another specific embodiment, the present invention also provides use of the protein in improving the photosynthetic efficiency of rice.

The present invention further provides a composition, which comprises:

(a) the promoter of one of two genes with different expression patterns and a coding region or non-coding RNA region of the other gene;

(b) a region between the promoter and the 5′ untranslated region of one of two genes with different expression patterns and a coding region or non-coding RNA region of the other gene;

(c) a localization signal region of one of the two protein coding genes with different subcellular localizations and a mature protein coding region of the other gene;

(d) gene coding regions of protein domains with different biological functions derived from two genes with different functions;

wherein, the composition is non-naturally occurring, and is directly connected on the biological chromosome and can be inherited stably.

In a specific embodiment, the “different expression patterns” refers to different levels of gene expression.

In another specific embodiment, the “different expression patterns” refers to different tissue-specific of gene expression.

In another specific embodiment, the “different expression patterns” refers to different developmental stage-specificities of gene expression.

In a specific embodiment, the “different subcellular locations” include, but are not limited to, nuclear location, cytoplasmic location, cell membrane location, chloroplast location, mitochondrial location, or endoplasmic reticulum membrane location.

In a specific embodiment, the “different biological functions” include, but are not limited to, recognition of specific DNA or RNA conserved sequence, activation of gene expression, binding to protein ligand, binding to small molecule signal, ion binding, or specific enzymatic reaction.

In a specific embodiment, the composition is fused in vivo.

The present invention also provides an editing method for regulating the gene expression level of a target endogenous gene in an organism, which is independent of an exogenous DNA donor fragment, which comprises the following steps:

simultaneously generating DNA breaks separately at selected sites between the promoter and the coding region of each of the target endogenous gene and an optional endogenous inducible or tissue-specific expression gene with a desired expression pattern; ligating the DNA breaks to each other by means of non-homologous end joining (NHEJ) or homologous repair, thereby generating an in vivo fusion of the coding region of the target endogenous gene and the optional inducible or tissue-specific expression promoter to form a new gene with expected expression patterns.

In a specific embodiment, the target endogenous gene and the optional endogenous inducible or tissue-specific expression gene with a desired expression pattern are located on the same chromosome or on different chromosomes.

In a specific embodiment, the target endogenous gene is yeast ERG9 gene, the endogenous inducible expression gene is HXT1 gene, and the inducible expression promoter is HXT1 in response to glucose concentration.

The present invention also provides a yeast endogenous inducible ERG9 gene obtainable by the editing method.

The present invention also provides use of the yeast endogenous inducible ERG9 gene in synthetic biology.

In particular, the present invention also provides an editing method of increasing the expression level of a target endogenous gene in an organism independent of an exogenous DNA donor fragment, which comprises the following steps: simultaneously generating DNA breaks at specific sites between the promoter and the CDS of each of the target endogenous gene and an optional endogenous highly-expressing gene; ligating the DNA breaks to each other via non-homologous end joining (NHEJ) or homologous repair to form an in vivo fusion of the coding region of the target endogenous gene and the optional strong endogenous promoter, thereby creating a new highly-expressing endogenous gene. This method is named as an editing method for knocking-up an endogenous gene.

In a specific embodiment, the target endogenous gene and the optional highly-expressing endogenous gene are located on the same chromosome.

In another specific embodiment, the target endogenous gene and the optional highly-expressing endogenous gene are located on different chromosomes.

In another aspect, the present invention provides an editing method for knocking up the expression of an endogenous HPPD gene in a plant, comprising fusing the coding region of the HPPD gene with a strong plant endogenous promoter in vivo to form a new highly-expressing plant endogenous HPPD gene. That is, simultaneously generating DNA breaks at specific sites between the promoter and the CDS of each of the HPPD gene and an optional endogenous highly-expressing gene, ligating the DNA breaks to each other through an intracellular repair pathway to form an in vivo fusion of the coding region of the HPPD gene and the optional endogenous strong promoter, thereby creating a new highly-expressing HPPD gene. In rice, the strong promoter is preferably a promoter of the ubiquitin2 gene.

The present invention also provides a highly-expressing plant endogenous HPPD gene obtainable by the above editing method.

The present invention also provides a highly-expressing rice endogenous HPPD gene which has a sequence selected from the group consisting of:

(1) a nucleic acid sequence as shown in SEQ ID NO: 9, SEQ ID NO: 10, SEQ ID NO: 18, SEQ ID NO: 19 or SEQ ID NO: 27 or a portion thereof or a complementary sequence thereof;

(2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

In another aspect, the present invention provides an editing method for knocking up the expression of an endogenous EPSPS gene in a plant, which comprises fusing the coding region of an EPSPS gene with a strong plant endogenous promoter in vivo to form a new highly-expressing plant endogenous EPSPS gene. That is, simultaneously generating DNA breaks at specific sites between the promoter and the CDS of each of the EPSPS gene and an optional highly-expressing endogenous gene, ligating the DNA breaks to each other through an intracellular repair pathway to form an in vivo fusion of the coding region of the EPSPS gene and the optional strong endogenous promoter, thereby creating a new highly-expressing EPSPS gene. In rice, the strong promoter is preferably a promoter of the TKT gene.

The present invention also provides a highly-expressing plant endogenous EPSPS gene obtainable by the above editing method.

The present invention also provides a highly-expressing rice endogenous EPSPS gene which has a sequence selected from the group consisting of:

(1) the nucleic acid sequence as shown in SEQ ID NO: 11, SEQ ID NO: 12, SEQ ID NO: 13 or SEQ ID NO: 14 or a partial sequence thereof or a complementary sequence thereof;

(2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

In another aspect, the present invention provides an editing method for knocking up the expression of an endogenous PPO (PPOX) gene in a plant, which comprises fusing the coding region of the PPO gene with a strong plant endogenous promoter in vivo to form a new highly-expressing plant endogenous PPO gene. That is, simultaneously generating DNA breaks at specific sites between the promoter and the CDS of each of the PPO gene and an optional highly-expressing endogenous gene, ligating the DNA breaks to each other through an intracellular repair pathway to form an in vivo fusion of the coding region of the PPO gene and the optional strong endogenous promoter, thereby creating a new highly-expressing PPO gene. In rice, the strong promoter is preferably a promoter of the CP12 gene. In Arabidopsis thaliana, the strong promoter is preferably a promoter of the ubiquitin10 gene.

The present invention also provides a highly-expressing plant endogenous PPO gene obtainable by the above editing method.

The present invention also provides a highly-expressing rice endogenous PPO1 gene having a sequence selected from the group consisting of:

(1) the nucleic acid sequence as shown in SEQ ID NO: 15, SEQ ID NO: 16, SEQ ID NO: 17, SEQ ID NO: 20, SEQ ID NO: 21, SEQ ID NO: 22, SEQ ID NO: 23, SEQ ID NO: 24, SEQ ID NO: 25, or SEQ ID NO: 26 or a partial sequence thereof or a complementary sequence thereof, (2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

The present invention also provides a highly-expressing rice endogenous PPO2 gene, which has a sequence selected from the group consisting of:

(1) the nucleic acid sequence as shown in SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO:41, SEQ ID NO: 42, SEQ ID NO: 43, SEQ ID NO: 44, SEQ ID NO: 45, SEQ ID NO: 46 or SEQ ID NO: 47, or a portion thereof or a complementary sequence thereof;

(2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

The present invention also provides a highly-expressing maize endogenous PPO2 gene, which has a sequence selected from the group consisting of:

(1) the nucleic acid sequence as shown in SEQ ID NO: 48 or SEQ ID NO: 49, or a portion thereof or a complementary sequence thereof;

(2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

The present invention also provides a highly-expressing wheat endogenous PPO2 gene, which has a sequence selected from the group consisting of:

(1) the nucleic acid sequence as shown in SEQ ID NO: 50, SEQ ID NO: 51, SEQ ID NO: 52, SEQ ID NO: 53, SEQ ID NO: 54, SEQ ID NO: 55 or SEQ ID NO: 56, or a portion thereof or a complementary sequence thereof;

(2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

The present invention also provides a highly-expressing oilseed rape endogenous PPO2 gene, which has a sequence selected from the group consisting of:

(1) the nucleic acid sequence as shown in SEQ ID NO: 57, SEQ ID NO: 58, SEQ ID NO: 59, SEQ ID NO: 60 or SEQ ID NO: 61, or a portion thereof or a complementary sequence thereof;

(2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

The present invention also provides use of the gene in the improvement of the resistance or tolerance to a corresponding inhibitory herbicide in a plant cell, a plant tissue, a plant part or a plant.

The present invention also provides a plant or a progeny derived therefrom regenerated from the plant cell which comprises the gene.

The present invention also provides a method for producing a plant with an increased resistance or tolerance to an herbicide, which comprises regenerating the plant cell which comprises the gene into a plant or a progeny derived therefrom.

In a specific embodiment, the plant with increased herbicide resistance or tolerance is a non-transgenic line obtainable by crossing a plant regenerated from the plant host cell of the invention with a wild-type plant to remove the exogenous transgenic component through genetic segregation.

The present invention also provides a herbicide-resistant rice, which comprises one or a combination of two or more of the rice new gene, highly-expressing rice endogenous HPPD gene, highly-expressing rice endogenous EPSPS gene, highly-expressing rice endogenous PPO1 gene, and highly-expressing rice endogenous PPO2 gene.

In a specific embodiment, the herbicide-resistant rice is non-transgenic.

The present invention also provides a maize, wheat or oilseed rape resistant to a herbicide, which comprises one or a combination of two or more of the maize new gene, the wheat or maize new gene, the highly-expressing maize PPO2 gene, the highly-expressing wheat PPO2 gene, and the highly-expressing oilseed rape PPO2 gene.

In a specific embodiment, the maize, wheat or oilseed rape is non-transgenic.

The present invention also provides a method for controlling a weed in a cultivation site of a plant, wherein the plant is selected from the group consisting of the plant, a plant prepared by the method, the rice, or the maize, wheat or oilseed rape, wherein the method comprises applying to the cultivation site one or more corresponding inhibitory herbicides in an amount for effectively controlling the weed.

The present invention also provides an editing method for knocking up the expression of an endogenous WAK gene in a plant, characterized in that it comprises fusing the coding region of the WAK gene with a strong endogenous promoter of a plant in vivo to form a new highly-expressing plant endogenous WAK gene. That is, simultaneously generating DNA breaks respectively in selected specific sites between the promoter and the coding region of each of the WAK gene and an optional endogenous highly-expressing gene, ligating the DNA breaks to each other through an intracellular repair pathway, generating in vivo a fusion of the coding region of the WAK gene and the optional strong endogenous promoter to form a new highly-expressing WAK gene.

The present invention also provides a highly-expressing plant endogenous WAK gene obtainable by the editing method.

The present invention also provides a highly-expressing rice WAK gene, which has a sequence selected from the group consisting of:

(1) the nucleic acid sequence as shown in SEQ ID NO: 62, SEQ ID NO: 63, SEQ ID NO: 64, SEQ ID NO: 65, SEQ ID NO: 66, SEQ ID NO: 67 or SEQ ID NO: 68, or a portion thereof or a complementary sequence thereof;

(2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

The present invention also provides an editing method for knocking up the expression of an endogenous CNGC gene in a plant, characterized in that it comprises fusing the coding region of the CNGC gene with a strong endogenous promoter of a plant in vivo to form a new highly-expressing plant endogenous CNGC gene. That is, simultaneously generating DNA breaks respectively in selected specific sites between the promoter and the coding region of each of the CNGC gene and an optional endogenous highly-expressing gene, ligating the DNA breaks to each other through an intracellular repair pathway, generating in vivo a fusion of the coding region of the CNGC gene and the optional strong endogenous promoter to form a new highly-expressing CNGC gene.

The present invention also provides a highly-expressing plant endogenous CNGC gene obtainable by the editing method.

The present invention also provides a highly-expressing rice CNGC gene, which has a sequence selected from the group consisting of:

(1) the nucleic acid sequence as shown in SEQ ID NO: 69, SEQ ID NO: 70, SEQ ID NO: 71 or SEQ ID NO: 72, or a portion thereof or a complementary sequence thereof;

(2) a sequence having an identity of at least 75%, at least 80%, at least 85%, at least 90%, at least 95%, at least 98% or at least 99% to any one of the sequences as defined in (1); or

(3) a nucleic acid sequence capable of hybridizing to the sequence as shown in (1) or (2) under a stringent condition.

The present invention also provides use of the gene in conferring or improving a resistance to rice blast in rice.

The present invention also provides a rice resistant to rice blast, which comprises one or a combination of two or more of the highly-expressing rice WAK gene, and the highly-expressing rice CNGC gene.

Preferably the rice is non-transgenic.

The present invention also provides an editing method for knocking up the expression of an endogenous GH1 gene in a fish, characterized in that it comprises fusing the coding region of the GH1 gene with a strong endogenous promoter of a fish in vivo to form a new highly-expressing fish endogenous GH1 gene. That is, simultaneously generating DNA breaks respectively in selected specific sites between the promoter and the coding region of each of the GH1 gene and an optional endogenous highly-expressing gene, ligating the DNA breaks to each other through an intracellular repair pathway, generating in vivo a fusion of the coding region of the GH1 gene and the optional strong endogenous promoter to form a new highly-expressing GH1 gene; the strong promoter is preferably the corresponding fish ColIA1a (Collagen type I alpha 1a) gene promoter, RPS15A (ribosomal protein S15a) gene promoter, Actin promoter or DDX5 [DEAD (Asp-Glu-Ala-Asp) box helicase 5] gene promoter.

The present invention also provides a highly-expressing fish endogenous GH1 gene obtainable by the editing method.

The present invention also provides use of the highly-expressing fish endogenous GH1 gene in fish breeding.

The present invention also provides an editing method for knocking up the expression of an endogenous IGF2 (Insulin-like growth factor 2) gene in a pig, characterized in that it comprises fusing the coding region of the IGF2 gene with a strong endogenous promoter of a pig in vivo to form a new highly-expressing pig endogenous IGF2 gene. That is, simultaneously generating DNA breaks respectively in selected specific sites between the promoter and the coding region of each of the IGF2 gene and an optional endogenous highly-expressing gene, ligating the DNA breaks to each other through an intracellular repair pathway, generating in vivo a fusion of the coding region of the IGF2 gene and the optional strong endogenous promoter to form a new highly-expressing IGF2 gene; the strong promoter is preferably one of the pig TNNI2 and TNNT3 gene promoter.

The present invention also provides a highly-expressing pig endogenous IGF2 gene obtainable by the editing method.

The present invention also provides use of the highly-expressing pig endogenous IGF2 gene in pig breeding.

The present invention also provides an editing method for knocking up the expression of an endogenous IGF1 (Insulin-like growth factor 1) gene in a chicken embryo fibroblast, characterized in that it comprises fusing the coding region of the IGF1 gene with a strong endogenous promoter of a chicken in vivo to form a new highly-expressing chicken endogenous IGF1 gene. That is, simultaneously generating DNA breaks respectively in selected specific sites between the promoter and the coding region of each of the IGF1 gene and an optional endogenous highly-expressing gene, ligating the DNA breaks to each other through an intracellular repair pathway, generating in vivo a fusion of the coding region of the IGF1 gene and the optional strong endogenous promoter to form a new highly-expressing IGF1 gene; the strong promoter is preferably chicken MYBPC1 (myosin binding protein C) gene promoter.

The present invention also provides a highly-expressing chicken endogenous IGF1 gene obtainable by the editing method.

The present invention also provides use of the highly-expressing chicken endogenous IGF1 gene in chicken breeding.

The present invention also provides an editing method for knocking up the expression of an endogenous EPO (Erythropoietin) gene in an animal cell, characterized in that it comprises fusing the coding region of the EPO gene with a strong endogenous promoter of an animal in vivo to form a new highly-expressing endogenous EPO gene. That is, simultaneously generating DNA breaks respectively in selected specific sites between the promoter and the coding region of each of the EPO gene and an optional endogenous highly-expressing gene, ligating the DNA breaks to each other through an intracellular repair pathway, generating in vivo a fusion of the coding region of the EPO gene and the optional strong endogenous promoter to form a new highly-expressing EPO gene.

The present invention also provides a highly-expressing animal endogenous EPO gene obtainable by the editing method.

The present invention also provides use of the highly-expressing animal endogenous EPO gene in animal breeding.

The present invention also provides an editing method for knocking up the expression of an endogenous p53 gene in an animal cell, characterized in that it comprises fusing the coding region of the p53 gene with a strong endogenous promoter of an animal in vivo to form a new highly-expressing endogenous p53 gene. That is, simultaneously generating DNA breaks respectively in selected specific sites between the promoter and the coding region of each of the p53 gene and an optional endogenous highly-expressing gene, ligating the DNA breaks to each other through an intracellular repair pathway, generating in vivo a fusion of the coding region of the p53 gene and the optional strong endogenous promoter to form a new highly-expressing p53 gene.

The present invention also provides a highly-expressing animal endogenous p53 gene obtainable by the editing method.

The present invention also provides use of the highly-expressing animal endogenous p53 gene in animal breeding or cancer prevention.

In a specific embodiment, the “DNA breaks” are produced by delivering a nuclease with targeting property into a cell of the organism to contact with the specific sites of the genomic DNA. There is no essential difference between this type of DNA breaks and the DNA breaks produced by traditional techniques (such as radiation or chemical mutagenesis).

In a specific embodiment, the “nuclease with targeting property” is selected from Meganuclease, Zinc finger nuclease (ZFN), TALEN and the CRISPR/Cas system.

Among them, the CRISPR/Cas system can generate two or more DNA double-strand breaks at different sites in the genome through two or more leading RNAs targeting different sequences; by separately designing the ZFN protein or TALEN protein in two or more specific site sequences, the Zinc finger nuclease and TALEN systems can simultaneously generate DNA double-strand breaks at two or more sites. When two breaks are located on the same chromosome, repair results such as deletion, inversion and doubling may occur; and when two breaks are located on two different chromosomes, crossover of chromosomal arms may occur. The deletion, inversion, doubling and exchange of chromosome segments at two DNA breaks can recombine different gene elements or protein domains, thereby creating a new functional gene.

In a specific embodiment, the said CRISPR/Cas system is Cas9 nuclease system or Cas12 nuclease system.

In a specific embodiment, the “nuclease with targeting property” exists in the form of DNA.

In another specific embodiment, the “nuclease with targeting property” exists in the form of mRNA or protein, rather than the form of DNA.

In a specific embodiment, the method for delivering the nucleases with targeting property into the cell is selected from a group consisting of: 1) PEG-mediated cell transfection; 2) liposome-mediated cell transfection; 3) electric shock transformation; 4) microinjection; 5) gene gun bombardment; 6) Agrobacterium-mediated transformation; 7) viral vector-mediated transformation method; or 8) nanomagnetic bead mediated transformation method.

The present invention also provides a DNA containing the gene.

The present invention also provides a protein encoded by the gene, or biologically active fragment thereof.

The present invention also provides a recombinant expression vector, which comprises the gene and a promoter operably linked thereto.

The present invention also provides an expression cassette containing the gene.

The present invention also provides a host cell, which comprises the expression cassette.

The present invention further provides an organism regenerated from the host cell.

In the research work of the inventors, it was found that in cells simultaneously undergoing dual-target or multi-target gene editing, a certain proportion of the ends of DNA double-strand breaks at different targets were spontaneously ligated to each other, resulting in events of deletion, inversion or duplication-doubling of the fragments between the targets on the same chromosome, and/or the exchange of chromosome fragments between targets on different chromosomes. It has been reported in the literature that this phenomenon commonly exists in plants and animals (Puchta et al. 2020. Changing local recombination patterns in Arabidopsis by CRISPR/Cas mediated chromosome engineering. Nat Commun. DOI: 10.1038/s41467-020-18277-z; Li et al. 2015. Efficient inversions and duplications of mammalian regulatory DNA elements and gene clusters by CRISPR/Cas9. J Mol Cell Biol. DOI: 10.1093/jmcb/mjv016).

The present inventors surprisingly discovered that, by inducing DNA double-strand breaks in a combination of gene editing targets near specific elements of a gene of interest, causing spontaneous repair ligation, directed combination of different gene elements can be achieved at the genome level without the need to provide a foreign DNA template, it is possible to produce therefrom a new functional gene. This strategy greatly accelerates the creation of new genes and has great potential in animal and plant breeding and gene function research.

### DETAILED DESCRIPTION OF INVENTION

In the present invention, unless otherwise specified, the scientific and technical terms used herein have the meanings commonly understood by those skilled in the art. In addition, protein and nucleic acid chemistry, molecular biology, cell and tissue culture, microbiology, immunology related terms and laboratory procedures used herein are all terms and routine procedures widely used in the corresponding fields. For example, the standard recombinant DNA and molecular cloning techniques used in the present invention are well known to those skilled in the art and are fully described in the following documents: Sambrook, J., Fritsch, E F and Maniatis, T., Molecular Cloning: A Laboratory Manual; Cold Spring Harbor Laboratory Press: Cold Spring Harbor, 1989. For a better understanding of the present invention, definitions and explanations of related terms are provided below.

The term “genome” as used herein refers to all complements of genetic material (genes and non-coding sequences) present in each cell or virus or organelle of an organism, and/or complete genome inherited from a parent as a unit (haploid).

Table A lists some of the ubiquitously-expressed genes and tissue-specific expressed genes in rice. Generally, in production applications, a DNA sequence within 3 kb upstream of the start codon of ubiquitously-expressed genes or tissue-specific genes is used as the promoter region and the 5′ non-coding region, where the promoter region of ubiquitously expressed genes is used as a representative of strong promoters, and the promoter region of tissue-specifically expressed genes is used as a representative of tissue-specific promoters. It is known that ubiquitously-expressed genes and tissue-specific genes in other species similar to rice can be found in public databases such as NCBI (https://www.ncbi.nlm.nih.gov), JGI (https://jgi.doe).gov/).

TABLE A
The ubiquitously-expressed genes and tissue-specific expressed genes in rice.

| Ubiquitously-expressed | |
|---|---|
| genes | Annotation of gene functions |
| LOC_Os02g06640 | ubiquitin family protein, putative, expressed |
| LOC_Os03g51600 | tubulin/FtsZ domain containing protein, putative, expressed |
| LOC_Os06g46770 | ubiquitin family protein, putative, expressed |
| LOC_Os11g43900 | translationally-controlled tumor protein, putative, expressed |
| LOC_Os01g67860 | fructose-bisphospate aldolase isozyme, putative, expressed |
| LOC_Os07g26690 | aquaporin protein, putative, expressed |
| LOC_Os03g27310 | histone H3, putative, expressed |
| LOC_Os05g41060 | ADP-ribosylation factor, putative, expressed |
| LOC_Os08g03290 | glyceraldehyde-3-phosphate dehydrogenase, putative, expressed |
| LOC_Os05g07700 | ribosomal protein, putative, expressed |
| LOC_Os03g08010 | elongation factor Tu, putative, expressed |
| LOC_Os02g48560 | fatty acid desaturase, putative, expressed |
| LOC_Os01g05490 | triosephosphate isomerase, cytosolic, putative, expressed |
| LOC_Os03g08020 | elongation factor Tu, putative, expressed |
| LOC_Os10g33800 | lactate/malate dehydrogenase, putative, expressed |
| LOC_Os06g04030 | histone H3, putative, expressed |
| LOC_Os04g57220 | ubiquitin-conjugating enzyme, putative, expressed |
| LOC_Os08g09250 | glyoxalase family protein, putative, expressed |
| LOC_Os03g08050 | elongation factor Tu, putative, expressed |
| LOC_Os08g02340 | 60S acidic ribosomal protein, putative, expressed |
| LOC_Os03g50885 | actin, putative, expressed |
| LOC_Os09g26420 | AP2 domain containing protein, expressed |
| LOC_Os03g12670 | expressed protein |
| LOC_Os05g49890 | ras-related protein, putative, expressed |
| LOC_Os05g06770 | 40S ribosomal protein S27a, putative, expressed |
| LOC_Os10g08550 | enolase, putative, expressed |
| LOC_Os04g53620 | ubiquitin family protein, putative, expressed |
| LOC_Os05g39960 | 40S ribosomal protein S26, putative, expressed |
| LOC_Os02g01560 | 40S ribosomal protein S4, putative, expressed |
| LOC_Os08g03640 | 60S acidic ribosomal protein P0, putative, expressed |
| LOC_Os06g23440 | eukaryotic translation initiation factor 1A, putative, expressed |
| LOC_Os10g32920 | ribosomal protein, putative, expressed |
| LOC_Os01g60410 | ubiquitin-conjugating enzyme, putative, expressed |
| LOC_Os01g22490 | 40S ribosomal protein S27a, putative, expressed |
| LOC_Os03g13170 | ubiquitin fusion protein, putative, expressed |
| Seed specificity highly | |
| expressed genes | MSU_Annotation |
| LOC_Os07g10580 | PROLM26-Prolamin precursor, expressed |
| LOC_Os01g55690 | glutelin, putative, expressed |
| LOC_Os10g26060 | glutelin, putative, expressed |
| LOC_Os07g11330 | RAL2-Seed allergenic protein RA5/RA14/RA17 precursor, |
| | expressed |
| LOC_Os07g11510 | RAL6-Seed allergenic protein RA5/RA14/RA17 precursor, |
| | expressed |
| LOC_Os05g41970 | SSAl-2S albumin seed storage family protein precursor, |
| | expressed |
| LOC_Os07g11380 | RAL4-Seed allergenic protein RA5/RA14/RA17 precursor, |
| | expressed |
| LOC_Os07g10570 | PROLM25-Prolamin precursor, expressed |
| LOC_Os02g16820 | glutelin, putative, expressed |
| LOC_Os02g25640 | glutelin, putative, expressed |
| LOC_Os02g16830 | glutelin, putative, expressed |
| LOC_Os02g15150 | glutelin, putative, expressed |
| LOC_Os03g31360 | glutelin, putative, expressed |
| LOC_Os02g15169 | glutelin, putative, expressed |
| LOC_Os02g15178 | glutelin, putative, expressed |
| LOC_Os06g31070 | PROLM24-Prolamin precursor, expressed |
| LOC_Os03g46100 | cupin domain containing protein, expressed |
| LOC_Os07g11410 | RAL5-Seed allergenic protein RA5/RA14/RA17 precursor, |
| | expressed |
| LOC_Os08g03410 | glutelin, putative, expressed |
| LOC_Os07g11920 | PROLM22-Prolamin precursor, expressed |
| LOC_Os07g11360 | RAL3-Seed allergenic protein RA5/RA14/RA17 precursor, |
| | expressed |
| LOC_Os03g57960 | cupin domain containing protein, expressed |
| LOC_Os11g33000 | SSA5-2S albumin seed storage family protein precursor, |
| | expressed |
| LOC_Os07g11650 | LTPL164-Protease inhibitor/seed storage/LTP family protein |
| | precursor, expressed |
| LOC_Os11g37270 | AMBP1-Antimicrobial peptide MBP-1 family protein precursor, |
| | expressed |
| LOC_Os07g11910 | PROLM20-Prolamin precursor, expressed |
| LOC_Os12g16890 | PROLM28-Prolamin precursor, expressed |
| LOC_Os07g11900 | PROLM19-Prolamin precursor, putative, expressed |
| LOC_Os02g15090 | glutelin, putative, expressed |
| LOC_Os08g08960 | Cupin domain containing protein, expressed |
| LOC_Os10g35050 | aquaporin protein, putative, expressed |
| LOC_Os04g46200 | oleosin, putative, expressed |
| LOC_Os05g35690 | GASR6-Gibberellin-regulated GASA/GAST/Snakin family |
| | protein precursor, expressed |
| LOC_Os07g11630 | LTPL163-Protease inhibitor/seed storage/LTP family protein |
| | precursor, expressed |
| LOC_Os05g26350 | PROLM4-Prolamin precursor, expressed |
| LOC_Os06g04200 | starch synthase, putative, expressed |
| LOC_Os05g26770 | PROLM18-Prolamin precursor, expressed |
| LOC_Os05g26720 | PROLM16-Prolamin precursor, expressed |
| LOC_Os10g39420 | CAMK_CAMK_like.8-CAMK includes calcium/calmodulin |
| | dependent protein kinases, expressed |
| LOC_Os04g33150 | desiccation-related protein PCC13-62 precursor, putative, |
| | expressed |
| LOC_Os06g51084 | 1,4-alpha-glucan-branching enzyme, chloroplast precursor, |
| | putative, expressed |
| LOC_Os06g46284 | glycosyl hydrolase, family 31, putative, expressed |
| Stamen specificity | |
| highly expressed genes | Annotation of gene functions |
| LOC_Os10g40090 | expansin precursor, putative, expressed |
| LOC_Os06g21410 | arabinogalactan peptide 23 precursor, putative, expressed |
| LOC_Os05g46530 | invertase/pectin methylesterase inhibitor family protein, putative, |
| | expressed |
| LOC_Os04g32680 | POEI20-Pollen Ole e I allergen and extensin family protein |
| | precursor, expressed |
| LOC_Os01g27190 | C2 domain containing protein, putative, expressed |
| LOC_Os06g17450 | expressed protein |
| LOC_Os01g69020 | retrotransposon protein, putative, unclassified, expressed |
| LOC_Os10g32810 | beta-amylase, putative, expressed |
| LOC_Os02g05670 | expressed protein |
| LOC_Os07g15530 | expressed protein |
| LOC_Os04g57280 | expressed protein |
| LOC_Os05g20570 | invertase/pectin methylesterase inhibitor family protein, putative, |
| | expressed |
| LOC_Os03g04770 | beta-amylase, putative, expressed |
| LOC_Os05g40740 | monocopper oxidase, putative, expressed |
| LOC_Os02g02450 | transposon protein, putative, unclassified, expressed |
| LOC_Os04g33710 | expressed protein |
| LOC_Os10g35930 | OsPLIM2c-LIM domain protein, putative actin-binding protein |
| | and transcription factor, expressed |
| LOC_Os06g03390 | expressed protein |
| LOC_Os02g03520 | THION25-Plant thionin family protein precursor, expressed |
| LOC_Os01g39970 | protein kinase domain containing protein, putative, expressed |
| LOC_Os12g42650 | pollen preferential protein, putative, expressed |
| LOC_Os08g02880 | CXXXC11-Cysteine-rich protein with paired CXXXC motifs |
| | precursor, expressed |
| LOC_Os10g17680 | profilin domain containing protein, expressed |
| LOC_Os01g21970 | protein kinase, putative, expressed |
| LOC_Os05g13850 | TsetseEP precursor, putative, expressed |
| LOC_Os04g57270 | expressed protein |
| LOC_Os02g26290 | fasciclin-like arabinogalactan protein 8 precursor, putative, |
| | expressed |
| LOC_Os07g13440 | RALFL12-Rapid ALkalinization Factor RALF family protein |
| | precursor, putative, expressed |
| LOC_Os10g17660 | profilin domain containing protein, expressed |
| LOC_Os04g25160 | pollen allergen, putative, expressed |
| LOC_Os05g13830 | TsetseEP precursor, putative, expressed |
| LOC_Os04g26220 | pollen allergen, putative, expressed |
| LOC_Os03g01610 | expansin precursor, putative, expressed |
| LOC_Os03g01650 | expansin precursor, putative, expressed |
| LOC_Os04g11130 | DEF9-Defensin and Defensin-like DEFL family, expressed |
| LOC_Os06g44470 | pollen allergen, putative, expressed |
| LOC_Os01g23880 | expressed protein |
| LOC_Os08g12520 | expressed protein |
| LOC_Os04g11195 | gamma-thionin family domain containing protein, expressed |
| Pistil specificity highly | |
| expressed genes | Annotation of gene functions |
| LOC_Os05g33150 | CHIT6-Chitinase family protein precursor, expressed |
| LOC_Os07g38130 | polygalacturonase inhibitor 1 precursor, putative, expressed |
| LOC_Os11g44810 | auxin-repressed protein, putative, expressed |
| LOC_Os09g37910 | HMG1/2, putative, expressed |
| LOC_Os01g42520 | expressed protein |
| LOC_Os12g38000 | 60S ribosomal protein L8, putative, expressed |
| LOC_Os03g08500 | AP2 domain containing protein, expressed |
| LOC_Os04g18090 | histone H1, putative, expressed |
| LOC_Os06g04030 | histone H3, putative, expressed |
| LOC_Os10g40730 | expansin precursor, putative, expressed |
| LOC_Os07g48910 | retrotransposon protein, putative, unclassified, expressed |
| LOC_Os03g22270 | auxin-repressed protein, putative, expressed |
| Leaf specificity highly | |
| expressed genes | Annotation of gene functions |
| LOC_Os12g17600 | ribulose bisphosphate carboxylase small chain, chloroplast |
| | precursor, putative, expressed |
| LOC_Os11g47970 | AAA-type ATPase family protein, putative, expressed |
| LOC_Os12g19381 | ribulose bisphosphate carboxylase small chain, chloroplast |
| | precursor, putative, expressed |
| LOC_Os11g07020 | fructose-bisphospate aldolase isozyme, putative, expressed |
| LOC_Os01g41710 | chlorophyll A-B binding protein, putative, expressed |
| LOC_Os09g17740 | chlorophyll A-B binding protein, putative, expressed |
| LOC_Os01g45274 | carbonic anhydrase, chloroplast precursor, putative, expressed |
| LOC_Os01g45914 | expressed protein |
| LOC_Os06g01210 | plastocyanin, chloroplast precursor, putative, expressed |
| LOC_Os08g10020 | photosystem II 10 kDa polypeptide, chloroplast precursor, |
| | putative, expressed |
| LOC_Os03g39610 | chlorophyll A-B binding protein, putative, expressed |
| LOC_Os01g31690 | oxygen-evolving enhancer protein 1, chloroplast precursor, |
| | putative, expressed |
| LOC_Os07g37240 | chlorophyll A-B binding protein, putative, expressed |
| LOC_Os07g37550 | chlorophyll A-B binding protein, putative, expressed |
| LOC_Os04g38600 | glyceraldehyde-3-phosphate dehydrogenase, putative, expressed |
| LOC_Os08g33820 | chlorophyll A-B binding protein, putative, expressed |
| LOC_Os11g13890 | chlorophyll A-B binding protein, putative, expressed |
| LOC_Os06g21590 | chlorophyll A-B binding protein, putative, expressed |
| LOC_Os02g10390 | chlorophyll A-B binding protein, putative, expressed |
| LOC_Os09g36680 | ribonuclease T2 family domain containing protein, expressed |
| LOC_Os08g44680 | photosystem I reaction center subunit II, chloroplast precursor, |
| | putative, expressed |
| LOC_Os12g19470 | ribulose bisphosphate carboxylase small chain, chloroplast |
| | precursor, putative, expressed |
| LOC_Os07g05480 | photosystem I reaction center subunit, chloroplast precursor, |
| | putative, expressed |
| LOC_Os07g04840 | PsbP, putative, expressed |
| LOC_Os08g01380 | 2Fe-2S iron-sulfur cluster binding domain containing protein, |
| | expressed |
| LOC_Os04g33830 | membrane protein, putative, expressed |
| LOC_Os05g48630 | expressed protein |
| LOC_Os01g52240 | chlorophyll A-B binding protein, putative, expressed |
| LOC_Os01g10400 | expressed protein |
| LOC_Os04g38410 | chlorophyll A-B binding protein, putative, expressed |
| LOC_Osl2g23200 | photosystem I reaction center subunit XI, chloroplast precursor, |
| | putative, expressed |
| LOC_Os07g38960 | chlorophyll A-B binding protein, putative, expressed |
| LOC_Os01g19740 | calvin cycle protein CP 12, putative, expressed |
| LOC_Os01g64960 | chlorophyll A-B binding protein, putative, expressed |
| LOC_Os03g03720 | glyceraldehyde-3-phosphate dehydrogenase, putative, expressed |
| LOC_Os12g08770 | photosystem I reaction center subunit N, chloroplast precursor, |
| | putative, expressed |
| LOC_Os02g02890 | peptidyl-prolyl cis-trans isomerase, putative, expressed |
| LOC_Os02g47020 | phosphoribulokinase/Uridine kinase family protein, expressed |
| LOC_Os07g25430 | photosystem I reaction center subunit IV A, chloroplast precursor, |
| | putative, expressed |
| LOC_Os01g17170 | magnesium-protoporphyrin IX monomethyl ester |
| | cyclase, chloroplast precursor, putative, expressed |
| LOC_Os07g36080 | oxygen evolving enhancer protein 3 domain containing protein, |
| | expressed |
| LOC_Os11g06720 | abscisic stress-ripening, putative, expressed |
| LOC_Os03g03910 | catalase domain containing protein, expressed |
| LOC_Os03g52840 | serine hydroxymethyltransferase, mitochondrial precursor, |
| | putative, expressed |
| LOC_Os12g08760 | carboxyvinyl-carboxyphosphonate phosphorylmutase, putative, |
| | expressed |
| LOC_Os05g41640 | phosphoglycerate kinase protein, putative, expressed |
| LOC_Os09g30340 | photosystem I reaction center subunit, chloroplast precursor, |
| | putative, expressed |
| LOC_Os04g21350 | flowering promoting factor-like 1, putative, expressed |
| LOC_Os04g16680 | fructose-1,6-bisphosphatase, putative, expressed |
| LOC_Os07g47640 | ultraviolet-B-repressible protein, putative, expressed |
| LOC_Os12g08730 | thioredoxin, putative, expressed |
| LOC_Os12g33120 | expressed protein |
| LOC_Os03g56670 | photosystem I reaction center subunit III, chloroplast precursor, |
| | putative, expressed |
| LOC_Os03g22370 | ultraviolet-B-repressible protein, putative, expressed |
| LOC_Os03g57220 | hydroxy acid oxidase 1, putative, expressed |
| LOC_Os01g56680 | photosystem II reaction center W protein, chloroplast precursor, |
| | putative, expressed |
| LOC_Os02g51080 | FAD binding domain containing protein, expressed |
| LOC_Os07g32880 | ATP synthase gamma chain, putative, expressed |
| LOC_Os03g17070 | ATP synthase B chain, chloroplast precursor, putative, expressed |
| LOC_Os01g13690 | ligA, putative, expressed |
| LOC_Os04g52260 | LTPL124-Protease inhibitor/seed storage/LTP family protein |
| | precursor, expressed |
| LOC_Os12g43600 | RNA recognition motif containing protein, expressed |
| LOC_Os01g51410 | glycine dehydrogenase, putative, expressed |
| LOC_Os06g40940 | glycine dehydrogenase, putative, expressed |
| LOC_Os06g15400 | expressed protein |
| LOC_Os12g02320 | LTPL12-Protease inhibitor/seed storage/LTP family protein |
| | precursor, expressed |
| LOC_Os07g01760 | aminotransferase, classes I and II, domain containing protein, |
| | expressed |
| LOC_Os08g39300 | aminotransferase, putative, expressed |
| LOC_Os06g04270 | transketolase, chloroplast precursor, putative, expressed |
| LOC_Os08g04500 | terpene synthase, putative, expressed |
| LOC_Os02g44630 | aquaporin protein, putative, expressed |
| LOC_Os12g23180 | 3-beta hydroxysteroid dehydrogenase/isomerase family protein, |
| | putative, expressed |
| LOC_Os06g51220 | HMG1/2, putative, expressed |
| LOC_Os04g41560 | B-box zinc finger family protein, putative, expressed |
| LOC_Os04g56400 | glutamine synthetase, catalytic domain containing protein, |
| | expressed |

Table B lists some functional genes that have been reported to be related to plant metabolites. Up-regulated expression of these genes or specific expression in fruits, leaves and other organs may enhance the economic value of such plants.

TABLE B
Genes related to secondary metabolites of plants.

| Plant | Gene name | Utility | Reference |
|---|---|---|---|
| Carex | CrUGT87A1 | Flavonoids, | Zhang, K., et al. (2021). “CrUGT87A1, a UDP-sugar |
| rigescens | | Salt | glycosyltransferases (UGTs) gene from Carex |
| | | tolerance | rigescens, increases salt tolerance by accumulating |
| | | | flavonoids for antioxidation in Arabidopsis thaliana.” |
| | | | Plant Physiol Biochem 159: 28-36. |
| Solanum | S1MYB14 | Flavonoids | Li, Z., et al. (2021). “S1MYB14 promotes flavonoids |
| lycopersicum | | | accumulation and confers higher tolerance to |
| | | | 2,4,6-trichlorophenol in tomato.” Plant Sci 303: |
| | | | 110796. |
| Citrus | CsPH4 | Proanthocyanidin | Zhang, Y., et al. (2020). “Citrus PH4-Noemi regulatory |
| | | | complex is involved in proanthocyanidin biosynthesis |
| | | | via a positive feedback loop.” J Exp Bot 71(4): |
| | | | 1306-1321. |
| Ginkgo | GbF3′H1 | Epigallocatechin, | Wu, Y., et al. (2020). “Overexpression of the GbF3′H1 |
| biloba | | Gallocatechin, | Gene Enhanced the Epigallocatechin, Gallocatechin, |
| L. | | and Catechin | and Catechin Contents in Transgenic Populus.” J Agric |
| | | | Food Chem 68(4): 998-1006. |
| L. | LrMYB1 | Flavonoids | Wang, C., et al. (2020). “Comparative transcriptome |
| ruthenicum | | | analysis of two contrasting wolfberry genotypes during |
| | | | fruit development and ripening and characterization of |
| | | | the LrMYB1 transcription factor that regulates |
| | | | flavonoid biosynthesis.” BMC Genomics 21(1): 295. |
| Citrus | CsCYT75B1 | Flavonoids, | Rao, M. J., et al. (2020). “CsCYT75B1, a Citrus |
| sinensis | | Drought | CYTOCHROME P450 Gene, Is Involved in |
| | | tolerance | Accumulation of Antioxidant Flavonoids and Induces |
| | | | Drought Tolerance in Transgenic Arabidopsis.” |
| | | | Antioxidants (Basel) 9(2). |
| Pear | PpMYB17 | Flavonoids | Premathilake, A.T., et al. (2020). “R2R3-MYB |
| | | | transcription factor PpMYB17 positively regulates |
| | | | flavonoid biosynthesis in pear fruit.” Planta 252(4): 59. |
| Raphanus | RsPAP2 | Anthocyanins | Fan, L., et al. (2020). “A genome-wide association |
| sativus | | | study uncovers a critical role of the RsPAP2 gene in |
| L. | | | red-skinned Raphanus sativus L.” Hortic Res 7: 164. |
| Pear | PbMYB12b | Flavonoids | Zhai, R., et al. (2019). “The MYB transcription factor |
| | | | PbMYB12b positively regulates flavonol biosynthesis |
| | | | in pear fruit.” BMC Plant Biol 19(1): 85. |
| Rosa | RrMYB5-/ | Flavonoids | Shen, Y., et al. (2019). “RrMYB5-and |
| rugosa | RrMYB10 | proanthocyanidin | RrMYB10-regulated flavonoid biosynthesis plays a |
| | | | pivotal role in feedback loop responding to wounding |
| | | | and oxidation in Rosa rugosa.” Plant Biotechnol J |
| | | | 17(11): 2078-2095. |
| Carthamus | CtCHI | Flavonoids | Liu, X., et al. (2019). “Molecular cloning and |
| tinctorius | | | functional characterization of chaicone isomerase from |
| | | | Carthamus tinctorius.” AMB Express 9(1): 132. |
| Salvia | SmANS | Anthocyanin | Li, H., et al. (2019). “Overexpression of SmANS |
| miltiorrhiza | | | Enhances Anthocyanin Accumulation and Alters |
| | | | Phenolic Acids Content in Salvia miltiorrhiza and |
| | | | Salvia miltiorrhiza Bge f alba Plantlets.” Int J Mol Sci |
| | | | 20(9). |
| Solanum | S1MYB75 | Anthocyanin | Jian, W., et al. (2019). “S1MYB75, an MYB-type |
| lycopersicum | | | transcription factor, promotes anthocyanin |
| | | | accumulation and enhances volatile aroma production |
| | | | in tomato fruits.” Hortic Res 6: 22. |
| Oryza | Lsi1 | Stresstolerance | Fang, C., et al. (2019). “Lsi1 modulates the antioxidant |
| sativa | | | capacity of rice and protects against ultraviolet-B |
| L. | | | radiation.” Plant Sci 278: 96-106. |
| Fraxinus | Fm4CL-like | Lignin | Chen, X., et al. (2019). “Molecular cloning and |
| mandschurica | 1 | | functional analysis of 4-Coumarate:CoA ligase |
| | | | 4(4CL-like 1)from Fraxinus mandshurica and its role in |
| | | | abiotic stress tolerance and cell wall synthesis.” BMC |
| | | | Plant Biol 19(1): 231. |
| Peach | PpMYB15/ | Flavonoids | Cao, Y., et al. (2019). “PpMYB15 and PpMYBF1 |
| | PpMYBF1 | | Transcription Factors Are Involved in Regulating |
| | | | Flavonol Biosynthesis in Peach Fruit.” J Agric Food |
| | | | Chem 67(2): 644-652. |
| Carthamus | CtCYP82G24 | Flavonoids | Ahmad, N., et al. (2019). “Overexpression of a Novel |
| tinctorius | | | Cytochrome P450 Promotes Flavonoid Biosynthesis |
| | | | and Osmotic Stress Tolerance in Transgenic |
| | | | Arabidopsis.” Genes (Basel) 10(10). |
| Vitis | VbDFR | Anthocyanins, | Zhu, Y., et al. (2018). “Molecular Cloning and |
| bellula | | Proanthocyanidins | Functional Characterization of a Dihydroflavonol |
| | | | 4-Reductase from Vitis bellula.” Molecules 23(4). |
| Ginkgo | GbMYBFL | Flavonoids | Zhang, W., et al. (2018). “Characterization and |
| biloba | | | functional analysis of a MYB gene (GbMYBFL) related |
| L. | | | to flavonoid accumulation in Ginkgo biloba.” Genes |
| | | | Genomics 40(1): 49-61. |
| Malus | MdWRKY11 | Flavonoids | Wang, N., et al. (2018). “Transcriptomic Analysis of |
| domestica | | | Red-Fleshed Apples Reveals the Novel Role of |
| | | | MdWRKY11 in Flavonoid and Anthocyanin |
| | | | Biosynthesis.” J Agric Food Chem 66(27): 7076-7086. |
| Gossypium | GhSPL10 | Flavonoids | Wang, L., et al. (2018). “The GhmiR157a-GhSPL10 |
| hirsutum | | | regulatory module controls initial cellular |
| | | | dedifferentiation and callus proliferation in cotton by |
| | | | modulating ethylene-mediated flavonoid biosynthesis.” |
| | | | J Exp Bot 69(5): 1081-1093. |
| Salvia | SmJMT | Phenolic | Wang, B., et al. (2018). “Molecular Characterization |
| miltiorrhiza | | acids | and Overexpression of SmJMT Increases the |
| | | | Production of Phenolic Acids in Salvia miltiorrhiza.” |
| | | | Int JMol Sci 19(12). |
| Malus | MdATG18a | Anthocyanin | Sun, X., et al. (2018). “MdATG18a overexpression |
| domestica | | | improves tolerance to nitrogen deficiency and regulates |
| | | | anthocyanin accumulation through increased autophagy |
| | | | in transgenic apple.” Plant Cell Environ 41(2): 469-480. |
| Citrus | UGTs | Flavonoids | Liu, X., et al. (2018). “Functional Characterization of a |
| sinensis | | | Flavonoid Glycosyltransferase in Sweet Orange (Citrus |
| | | | sinensis).” Front Plant Sci 9: 166. |
| Arabidopsis | UGT76E11 | Flavonoids | Li, Q., et al. (2018). “Ectopic expression of |
| thaliana | | | glycosyltransferase UGT76E11 increases flavonoid |
| | | | accumulation and enhances abiotic stress tolerance in |
| | | | Arabidopsis.” Plant Biol (Stuttg) 20(1): 10-19. |
| Arabidopsis | AtMYB12 | Flavonoids | Bhatia, C., et al. (2018). “Low Temperature-Enhanced |
| thaliana | | | Flavonol Synthesis Requires Light-Associated |
| | | | Regulatory Components in Arabidopsis thaliana.” Plant |
| | | | Cell Physiol 59(10): 2099-2112. |
| Antirrhinum | AmDEL | Flavonoids | Wang, F., et al. (2016). “The Antirrhinum AmDEL |
| | | | gene enhances flavonoids accumulation and salt and |
| | | | drought tolerance in transgenic Arabidopsis.” Planta |
| | | | 244(1): 59-73. |
| Lycium | LcF3H | Flavonoids | Song, X., et al. (2016). “Molecular cloning and |
| chinense | | Drought | identification of a flavanone 3-hydroxylase gene from |
| | | tolerance | Lycium chinense, and its overexpression enhances |
| | | | drought stress in tobacco.” Plant Physiol Biochem 98: |
| | | | 89-100. |
| Sorghum | SbMyb60 | Phenylpropanoid | Scully, E.D., et al. (2016). “Overexpression of |
| bicolor | | Drought | SbMyb60 impacts phenylpropanoid biosynthesis and |
| | | tolerance | alters secondary cell wall composition in Sorghum |
| | | | bicolor.” Plant J 85(3): 378-395. |
| Vitis | VvibZIPC22 | Flavonoids | Malacame, G., et al. (2016). “The grapevine |
| vinifera | | | VvibZIPC22 transcription factor is involved in the |
| | | | regulation of flavonoid biosynthesis.” J Exp Bot |
| | | | 67(11): 3509-3522. |
| Eupatorium | EaCHS1 | Flavonoids | Lijuan, C., et al. (2015). “Chaicone synthase EaCHSI |
| adenophorum | | | from Eupatorium adenophorum functions in salt stress |
| | | | tolerance in tobacco.” Plant Cell Rep 34(5): 885-894. |
| Arabidopsis | AtROS1 | Flavonoids | Bharti, P., et al. (2015). “AtROS1 overexpression |
| thaliana | | | provides evidence for epigenetic regulation of genes |
| | | | encoding enzymes of flavonoid biosynthesis and |
| | | | antioxidant pathways during salt stress in transgenic |
| | | | tobacco.” J Exp Bot 66(19): 5959-5969. |
| Malus | MdMYB9/ | Anthocyanin, | An, X.H., et al. (2015). “MdMYB9 and MdMYB11 are |
| domestica | MdMYB11 | proanthocyanidin | involved in the regulation of the JA-induced |
| | | | biosynthesis of anthocyanin and proanthocyanidin in |
| | | | apples.” Plant Cell Physiol 56(4): 650-662. |
| Arabidopsis | PAP1 | Flavonoids | Mitsunami, T., et al. (2014). “Overexpression of the |
| thaliana | | | PAP1 transcription factor reveals a complex regulation |
| | | | of flavonoid and phenylpropanoid metabolism in |
| | | | Nicotiana tabacum plants attacked by Spodoptera |
| | | | litura.” PLoS One 9(9): el08849. |
| Carnellia | CsF3H | Flavonoids | Mahajan, M. and S. K. Yadav (2014). “Overexpression |
| sinensis | | | of a tea flavanone 3-hydroxylase gene confers tolerance |
| | | | to salt stress and Alternaria solani in transgenic |
| | | | tobacco.” Plant Mol Biol 85(6): 551-573. |
| Arabidopsis | UVR8 | Flavonoids | Fasano, R., et al. (2014). “Role of Arabidopsis UV |
| thaliana | | | RESISTANCE LOCUS 8 in plant growth reduction |
| | | | under osmotic stress and low levels of UV-B.” Mol |
| | | | Plant 7(5): 773-791. |
| Fagopyrum | FtMYB1/ | Proanthocyanidins | Bai, Y.C., et al. (2014). “Characterization of two |
| tataricum | FtMYB2, | | tartary buckwheat R2R3-MYB transcription factors and |
| Gaertn | | | their regulation of proanthocyanidin biosynthesis.” |
| | | | Physiol Plant 152(3): 431-440. |
| Ipomoea | IbDFR | Anthocyanin | Wang, H., et al. (2013). “Functional characterization of |
| batatas | | | Dihydroflavonol-4-reductase in anthocyanin |
| Lam. | | | biosynthesis of purple sweet potato underlies the direct |
| | | | evidence of anthocyanins function against abiotic |
| | | | stresses.” PLoS One 8(11): e78484. |
| Theobroma | TcANR/ | Proanthocyanidin | Liu, Y., et al. (2013). “Proanthocyanidin synthesis in |
| cacao | TcLAR | | Theobroma cacao: genes encoding anthocyanidin |
| | | | synthase, anthocyanidin reductase, and |
| | | | leucoanthocyanidin reductase.” BMC Plant Biol 13: |
| | | | 202. |
| Solanum | DFR | Flavonoids | Kostyn, K., et al. (2013). “Transgenic potato plants |
| tuberosum | | vitamin C | with overexpression of dihydroflavonol reductase can |
| | | | serve as efficient nutrition sources.” J Agric Food |
| | | | Chem 61(27): 6743-6753. |
| Epimedium | EsMYBA1 | Anthocyanin | Huang, W., et al. (2013). “A R2R3-MYB transcription |
| sagittatum | | | factor from Epimedium sagittatum regulates the |
| | | | flavonoid biosynthetic pathway.” PLoS One 8(8): |
| | | | e70778. |
| Gentiana | GtMYBP3/ | Flavonoids | Nakatsuka, T., et al. (2012). “Isolation and |
| triflora | GtMYBP4 | | characterization of GtMYBP3 and GtMYBP4, |
| | | | orthologues of R2R3-MYB transcription factors that |
| | | | regulate early flavonoid biosynthesis, in gentian |
| | | | flowers.” J Exp Bot 63(18): 6505-6517. |
| Triticum | TaMYB4 | Flavonoids | Ma, Q.H., et al. (2011). “TaMYB4 cloned from wheat |
| aestivum L. | | | regulates lignin biosynthesis through negatively |
| | | | controlling the transcripts of both cinnamyl alcohol |
| | | | dehydrogenase and cinnamoyl-CoA reductase genes.” |
| | | | Biochimie 93(7): 1179-1186. |
| Fragaria | EGS/IGS | Eugenol, | Hoffmann, T., et al. (2011). “Metabolic engineering in |
| vesca | | Isoeugenol | strawberry fruit uncovers a dormant biosynthetic |
| | | | pathway.” Metab Eng 13(5): 527-531. |
| Populus | MYB134 | Proanthocyanidins | Mellway, R.D., et al. (2009). “The wound-, pathogen-, |
| spp. | | | and ultraviolet B-responsive MYB134 gene encodes an |
| | | | R2R3 MYB transcription factor that regulates |
| | | | proanthocyanidin synthesis in poplar.” Plant Physiol |
| | | | 150(2): 924-941. |
| Saussure | CHI | Apigenin | Li, F.X., et al. (2006). “Overexpression of the |
| amedusa | | | Saussurea medusa chaicone isomerase gene in S. |
| | | | involucrata hairy root cultures enhances their |
| | | | biosynthesis of apigenin.” Phytochemistry 67(6): |
| | | | 553-560. |
| Vitis | VvMYB5a | Phenolic | Deluc, L., et al. (2006). “Characterization of a |
| vinifera | | compounds | grapevine R2R3-MYB transcription factor that |
| | | | regulates the phenylpropanoid pathway.” Plant Physiol |
| | | | 140(2): 499-511. |
| Medicago | MtDFR1 | Flavonoids | Xie, D.Y., et al. (2004). “Molecular and biochemical |
| truncatula | | | analysis of two cDNA clones encoding |
| | | | dihydroflavonol-4-reductase from Medicago |
| | | | truncatula.” Plant Physiol 134(3): 979-994. |
| Solanum | CHS/CHI/DFR | Phenolic acids, | Lukaszewicz, M., et al. (2004). “Antioxidant capacity |
| tuberosum L. | | Anthocyanins | manipulation in transgenic potato tuber by changes in |
| | | | phenolic compounds content.” J Agric Food Chem |
| | | | 52(6): 1526-1533. |
| Zea | LC/C1 | Flavonoids | Le Gall, G., et al. (2003). “Characterization and content |
| mays L. | | | of flavonoid glycosides in genetically modified tomato |
| | | | (Lycopersicon esculentum) fruits.” J Agric Food Chem |
| | | | 51(9): 2438-2446. |
| Petunia | Petunia chi-a | Flavonoids | Muir, S.R., et al. (2001). “Overexpression of petunia |
| | | | chaicone isomerase in tomato results in fruit containing |
| | | | increased levels of flavonols.” Nat Biotechnol 19(5): |
| | | | 470-474. |
| Taxus | TcCYP725A | Taxol | Liao, W., et al. (2019). “Sub-cellular localization and |
| chinensis | 22 | | overexpressing analysis of hydroxylase gene |
| | | | TcCYP725A22 of Taxus chinensis.” Sheng Wu Gong |
| | | | Cheng Xue Bao 35(6): 1109-1116. |
| Lycopersicon | MI0X4 | Vitamin C | Munir, S., et al. (2020). “Genome-wide analysis of |
| esculenturn | | | Myo-inositol oxygenase gene family in tomato reveals |
| | | | their involvement in ascorbic acid accumulation.” BMC |
| | | | Genomics 21(1): 284. |
| Zea mays L./ | ZmPTPN | Vitamin C, | Zhang, H., et al. (2020). “Enhanced Vitamin C |
| Arabidopsis | AtPTPN | Drought | Production Mediated by an ABA-Induced PTP-like |
| thaliana | | tolerance | Nucleotidase Improves Plant Drought Tolerance in |
| | | | Arabidopsis and Maize.” Mol Plant 13(5): 760-776. |
| Elaeis | EgHGGT | Vitamin E | Luo, T., et al. (2020). “Identifying Vitamin E |
| guineensis | | | Biosynthesis Genes in Elaeis guineensis by |
| | | | Genome-Wide Association Study.” J Agric Food Chem |
| | | | 68(2): 678-685. |
| Zea | ZmTMT | Vitamin E | Zhang, L., et al. (2020). “Overexpression of the maize |
| mays | | | gamma-tocopherol methyltransferase gene (ZmTMT) |
| L. | | | increases alpha-tocopherol content in transgenic |
| | | | Arabidopsis and maize seeds.” Transgenic Res 29(1): |
| | | | 95-104. |
| Zea | ZmPORB2 | Vitamin E | Zhan, W., et al. (2019). “An allele of ZmPORB2 |
| mays | | | encoding a protochlorophyllide oxidoreductase |
| L. | | | promotes tocopherol accumulation in both leaves and |
| | | | kernels of maize.” Plant J 100(1): 114-127. |
| Pyrus | PbrWRKY53 | Vitamin C | Liu, Y., et al. (2019). “A WRKY transcription factor |
| betulaefolia | | | PbrWRKY53 from Pyrus betulaefolia is involved in |
| | | | drought tolerance and As A accumulation.” Plant |
| | | | Biotechnol J 17(9): 1770-1787. |
| Arabidopsis | PDX-II | Vitamin B6 | Bagri, D.S., et al. (2018). “Overexpression of PDX-II |
| thaliana. | | | gene in potato (Solanum tuberosum L.) leads to the |
| | | | enhanced accumulation of vitamin B6 in tuber tissues |
| | | | and tolerance to abiotic stresses.” Plant Sci 272: |
| | | | 267-275. |
| Brassica | BjHMGS1 | Vitamin E | Liao, P., et al. (2018). “Improved fruit |
| juncea | | | alpha-tocopherol, carotenoid, squalene and phytosterol |
| | | | contents through manipulation of Brassica juncea |
| | | | 3-HYDROXY-3-METHYLGLUTARYL-COA |
| | | | SYNTHASE1 in transgenic tomato.” Plant Biotechnol J |
| | | | 16(3): 784-796. |
| Hordeum | HvHGGT | Vitamin E | Chen, J., et al. (2017). “Overexpression of HvHGGT |
| vulgare L. | | | Enhances Tocotrienol Levels and Antioxidant Activity |
| | | | in Barley.” J Agric Food Chem 65(25): 5181-5187. |
| Medicago | MsHPPD | Vitamin E | Jiang, J., et al. (2017). |
| sativa L. | | | “P -HYDROXYPHENYLPYRUVATE |
| | | | DIOXYGENASE from Medicago sativa is involved in |
| | | | vitamin E biosynthesis and abscisic acid-mediated seed |
| | | | germination.” Sci Rep 7: 40625. |
| Arabidopsis | AtOxR | Vitamin C | Bu, Y., et al. (2016). “Overexpression of AtOxR gene |
| thaliana | | | improves abiotic stresses tolerance and vitamin C |
| | | | content in Arabidopsis thaliana.” BMC Biotechnol |
| | | | 16(1): 69. |
| Medicago | MsTMT | Vitamin E | Jiang, J., et al. (2016). “Overexpression of Medicago |
| sativa L. | | | sativa TMT elevates the alpha-tocopherol content in |
| | | | Arabidopsis seeds, alfalfa leaves, and delays |
| | | | dark-induced leaf senescence.” Plant Sci 249: 93-104. |
| Arabidopsis | AtGCHI | Vitamin B9 | Ramirez Rivera, N. G., et al. (2016). “Metabolic |
| thaliana | | | engineering of folate and its precursors in Mexican |
| | | | common bean (Phaseolus vulgaris L.).” Plant |
| | | | Biotechnol J 14(10): 2021-2032. |
| Lactuca | LsMT | Vitamin E | Tang, Y., et al. (2016). “Roles of MPBQ-MT in |
| sativa | | | Promoting alpha/gamma-Tocopherol Production and |
| | | | Photosynthesis under High Light in Lettuce.” PLoS |
| | | | One 11(2): e0148490. |
| Arabidopsis | VTE6 | Vitamin E | Vom Dorp, K., et al. (2015). “Remobilization of Phytol |
| thaliana | | | from Chlorophyll Degradation Is Essential for |
| | | | Tocopherol Synthesis and Growth of Arabidopsis.” |
| | | | Plant Cell 27(10): 2846-2859. |
| Triticum | CrtB | Beta-carotene | Zeng, J., et al. (2015). “Metabolic Engineering of |
| aestivum L. | | | Wheat Provitamin A by Simultaneously Overexpressing |
| | | | CrtB and Silencing Carotenoid Hydroxylase (TaHYD).” |
| | | | J Agric Food Chem 63(41): 9083-9092. |
| Solanum | SIVKOR | Drought | Yu, Z.B., et al. (2016). “A homologue of vitamin K |
| lycopersicum | | tolerance | epoxide reductase in Solanum lycopersicum is involved |
| | | Salt | in resistance to osmotic stress.” Physiol Plant 156(3): |
| | | tolerance | 311-322. |
| Oryza | GTPCHI/ | Vitamin B9/ | Dong, W., et al. (2014). “Overexpression of folate |
| sativa | ADCS/DHFS/ | folate | biosynthesis genes in rice (Oryza sativa L.) and |
| L. | FPGS | | evaluation of their impact on seed folate content.” Plant |
| | | | Foods HumNutr 69(4): 379-385. |
| Strawberry | FaGalUR | Vitamin C | Amaya, I., et al. (2015). “Increased antioxidant capacity |
| | | | in tomato by ectopic expression of the strawberry |
| | | | D-galacturonate reductase gene.” Biotechnol J 10(3): |
| | | | 490-500. |
| Arabidopsis | myo-inositol | Vitamin C | Lisko, K.A., et al. (2013). “Elevating vitamin C |
| thaliana | oxygenase/ | | content via overexpression of myo-inositol oxygenase |
| | 1-gulono-1,4- | | and 1-gulono-1,4-lactone oxidase in Arabidopsis leads |
| | lactone | | to enhanced biomass and tolerance to abiotic stresses.” |
| | oxidase | | In Vitro Cell Dev Biol Plant 49(6): 643-655. |
| Perilla | TMT | Vitamin E | Arun, M., et al. (2014). “Transfer and targeted |
| frutescens | | | overexpression of gamma-tocopherol methyltransferase |
| | | | (gamma-TMT) gene using seed-specific promoter |
| | | | improves tocopherol composition in Indian soybean |
| | | | cultivars.” Appl Biochem Biotechnol 172(4): |
| | | | 1763-1776. |
| Arabidopsis | AtTMT | Vitamin E | Zhang, G.Y., et al. (2013). “Increased |
| thaliana | | | alpha-tocotrienol content in seeds of transgenic rice |
| | | | overexpressing Arabidopsis gamma-tocopherol |
| | | | methyltransferase.” Transgenic Res 22(1): 89-99. |
| Oryza | NAS | Increase Zn | Johnson, A.A., et al. (2011). “Constitutive |
| sativa | | Fe | overexpression of the OsNAS gene family reveals |
| L. | | | single-gene strategies for effective iron- and |
| | | | zinc-biofortification of rice endosperm.” PLoS One |
| | | | 6(9): e24476. |
| Zea | crtB/crt1 | Vitamin A | Aluru, M., et al. (2008). “Generation of transgenic |
| mays | | | maize with enhanced provitamin A content.” J Exp Bot |
| L. | | | 59(13): 3551-3562. |
| Arabidopsis | PT/V-TE2 & | Vitamin E | Lee, K., et al. (2007). “Overexpression of Arabidopsis |
| thaliana | TC/VTE1 | | homogentisate phytyltransferase or tocopherol cyclase |
| | | | elevates vitamin E content by increasing |
| | | | gamma-tocopherol level in lettuce (Lactuca sativa L.).” |
| | | | Mol Cells 24(2): 301-306. |
| Arabidopsis | VTE1 | Vitamin E | Kanwischer, M., et al. (2005). “Alterations in |
| thaliana | | | tocopherol cyclase activity in transgenic and mutant |
| | | | plants of Arabidopsis affect tocopherol content, |
| | | | tocopherol composition, and oxidative stress.” Plant |
| | | | Physiol 137(2): 713-723. |
| Hordeum | 4-hydroxyphenyl- | Vitamin E | Falk, J., et al. (2003). “Constitutive overexpression of |
| vulgare | pyruvate | | barley 4-hydroxyphenylpyruvate dioxygenase in |
| L. | dioxygenase | | tobacco results in elevation of the vitamin E content in |
| | | | seeds but not in leaves.” FEBS Lett 540(1-3): 35-40. |
| Triticum | DHAR | Vitamin C | Chen, Z., et al. (2003). “Increasing vitamin C content of |
| aestivum L. | | | plants through enhanced ascorbate recycling.” Proc |
| | | | Natl Acad Sci U S A 100(6): 3525-3530. |
| Strawberry | GalUR | Vitamin C | Agius, F., et al. (2003). “Engineering increased vitamin |
| | | | C levels in plants by overexpression of a |
| | | | D-galacturonic acid reductase.” Nat Biotechnol 21(2): |
| | | | 177-181. |
| Arabidopsis | gamma-tocopherol | Vitamin E | Shintani, D. and D. DellaPenna (1998). “Elevating the |
| thaliana | methyltransferase | | vitamin E content of plants through metabolic |
| | | | engineering.” Science 282(5396): 2098-2100. |

Table C lists the important functional genes in oilseed rape. The combination of such genes with those endogenous promoters of oilseed rape can be used to create non-transgenic endogenous high-expression new genes or tissue-specific expression genes by applying the method in the present invention to bring about more application scenarios for breeding. There are also a large number of genes with reported functions in rice, corn, wheat, soybeans and other species. For those functional genes or non-coding RNAs that need to be up-regulated to realize competitive advantages for crops, their combinations with known strong expression promoters are available for creating customized new genes with new expression patterns as per needed by using the method in the present invention.

TABLE C
Important functional genes in oilseed rape

| Gene name | Application | Reference |
|---|---|---|
| Metallothionein | To improve tolerance to | Pan, Y., et al. (2018). “Genome-Wide |
| Family | heavy metal toxicity | Characterization and Analysis of Metallothionein |
| Genes (MT)- | | Family Genes That Function in Metal Stress |
| metallothionein | | Tolerance in Brassica napus L.” Int J Mol Sci |
| | | 19(8). |
| Alternative | To confer tolerance to | Yang, H., et al. (2019). “Overexpression of |
| oxidases | osmotic and salt stress | BnaAOX1b Confers Tolerance to Osmotic and Salt |
| (AOXs) | in oilseed rape | Stress in Rapeseed.” G3 (Bethesda) 9(10): |
| | | 3501-3511. |
| CBF/DREB1- | To improve freezing | Savitch, L. V., et al. (2005). “The effect of |
| like | tolerance and regulate | overexpression of two Brassica CBF/DREB1-like |
| transcription | chloroplast | transcription factors on photosynthetic capacity and |
| factors | development, thus to | freezing tolerance in Brassica napus.” Plant Cell |
| (BnCBF5 and 17) | improve photochemical | Physiol 46(9): 1525-1539. |
| | efficiency and | |
| | photosynthetic capacity | |
| Mitogen- | To indicate the | Wang, Z., et al. (2021). “Genome-Wide |
| activated protein | transcriptional level of | Identification and Analysis of MKK and MAPK |
| kinase | BnaMKK and | Gene Families in Brassica Species and Response to |
| (MAPK), Mito | BnaMAPK is usually | Stress in Brassica napus.” Int J Mol Sci 22(2). |
| gen-activated | regulated by growth, | |
| protein kinase | development and stress | |
| (MAPK) | signal. | |
| Family Genes | | |
| pyrab actin | Abiotic stress response | Di, F., et al. (2018). “Genome-Wide Analysis of the |
| resistance | | PYL Gene Family and Identification of PYL Genes |
| 1-like | | That Respond to Abiotic Stress in Brassica napus.” |
| (PYR/PYL) | | Genes (Basel) 9(3). |
| protein gene | | |
| family | | |
| BnPCS1; | Key factors in cadmium | Ding, Y., et al. (2018). “Screening of candidate |
| BnHMAs | stress response | gene responses to cadmium stress by RNA |
| | | sequencing in oilseed rape (Brassica napus L.).” |
| | | Environ Sci Pollut Res Int 25(32): 32433-32446. |
| APETALA2/e | Cold stress response | Du, C., et al. (2016). “Dynamic transcriptome |
| thylene | | analysis reveals AP2/ERF transcription factors |
| response | | responsible for cold stress in rapeseed (Brassica |
| factor | | napus L.).” Mol Genet Genomics 291(3): |
| (AP2/ERF) | | 1053-1067. |
| transcription | | |
| factor (TF) | | |
| superfamily | | |
| dehydrin, | Cold stress response | Edrisi Maryan, K., et al. (2019). “Analysis of |
| DHNs | | Brassica napus dehydrins and their Co-Expression |
| | | regulatory networks in relation to cold stress.” |
| | | Gene Expr Patterns 31: 7-17. |
| WRKY | To adapt to low boron | Feng, Y., et al. (2020). “Transcription factor |
| transcription | environmental stress | BnaA9.WRKY47 contributes to the adaptation of |
| factor | | Brassica napus to low boron stress by up-regulating |
| families; | | the boric acid channel gene BnaA3.NIP5; 1.” Plant |
| NIP5.1 | | Biotechnol J 18(5): 1241-1254. |
| phosphatidylinositol- | Drought resistance, | Georges, F., et al. (2009). “Over-expression of |
| phospholipase C2 | early flowering and | Brassica napus phosphatidylinositol-phospholipase |
| | maturation | C2 in canola induces significant changes in gene |
| | | expression and phytohormone distribution patterns, |
| | | enhances drought tolerance and promotes early |
| | | flowering and maturation.” Plant Cell Environ |
| | | 32(12): 1664-1681. |
| GRAS gene | Root stress response | Guo, P., et al. (2019). “Genome-wide survey and |
| family | | expression analyses of the GRAS gene family in |
| | | Brassica napus reveals their roles in root |
| | | development and stress response.” Planta 250(4): |
| | | 1051-1072. |
| Annexins | Cold stress response | He, X., et al. (2020). “Comprehensive analyses of |
| (ANN) | | the annexin (ANN) gene family in Brassica rapa, |
| genes | | Brassica oleracea and Brassica napus reveals their |
| | | roles in stress response.” Sci Rep 10(1): 4295. |
| CaM | Abiotic stress response | He, X., et al. (2020). “Genome-wide identification |
| (Calmodulin)/ | genes | and expression analysis of CaM/CML genes in |
| CML | | Brassica napus under abiotic stress.” J Plant Physiol |
| (calmodulin-like) | | 255: 153251. |
| genes | | |
| WRINKLED1, | Heat tolerance | Huang, R., et al. (2019). “Heat Stress Suppresses |
| BnWRI1 | | Brassica napus Seed Oil Accumulation by |
| | | Inhibition of Photosynthesis and BnWRI1 |
| | | Pathway.” Plant Cell Physiol 60(7): 1457-1470. |
| WAX | To promote growth and | Liu, N., et al. (2019). “Overexpression of WAX |
| INDUCER1/ | increase oil content | INDUCER1/SHINE1 Gene Enhances Wax |
| SHINE1 | | Accumulation under Osmotic Stress and Oil |
| (WIN1) | | Synthesis in Brassica napus.” Int J Mol Sci 20(18). |
| Cytokinin | Relates to pod length | Liu, P., et al. (2018). “Genome-Wide Identification |
| oxidase/ | | and Expression Profiling of Cytokinin |
| dehydrogenases | | Oxidase/Dehydrogenase (CKX) Genes Reveal |
| (CKXs) | | Likely Roles in Pod Development and Stress |
| | | Responses in Oilseed Rape (Brassica napus L.).” |
| | | Genes (Basel) 9(3). |
| mitogen-activated | Disease resistance | Wang, Z., et al. (2009). “Overexpression of |
| protein kinases 4, | | Brassica napus MPK4 enhances resistance to |
| MAPK4 | | Sclerotinia sclerotiorum in oilseed rape.” Mol Plant |
| | | Microbe Interact 22(3): 235-244. |
| ABSCISIC | Stress response | Xu, P. and W. Cai (2019). “Function of Brassica |
| ACID | | napus BnABI3 in Arabidopsis gs1, an Allele of |
| INSENSITIVE3 | | AtABI3, in Seed Development and Stress |
| | | Response.” Front Plant Sci 10: 67. |
| Alternative | Tolerance to salt stress | Yang, H., et al. (2019). “Overexpression of |
| oxidases | | BnaAOXlb Confers Tolerance to Osmotic and Salt |
| (AOXs) | | Stress in Rapeseed.” G3 (Bethesda) 9(10): |
| | | 3501-3511. |
| Glucosinolate | Resistance to | Zhang, Y., et al. (2015). “Overexpression of Three |
| Biosynthesis | Sclerotinia sclerotiorum | Glucosinolate Biosynthesis Genes in Brassica |
| | and Botrytis cinerea | napus Identifies Enhanced Resistance to Sclerotinia |
| | | sclerotiorum and Botrytis cinerea.” PLoS One |
| | | 10(10): e0140491. |
| tropinone | Cold resistance | Huang, Y., et al. (2020). “A Brassica napus |
| reductase | | Reductase Gene Dissected by Associative |
| | | Transcriptomics Enhances Plant Adaption to |
| | | Freezing Stress.” Front Plant Sci 11: 971. |
| aminoalcohol | Cold resistance | Qi, Q., et al. (2003). “Molecular and biochemical |
| phosphotransferase | | characterization of an |
| (AAPT1) | | aminoalcoholphosphotransferase (AAPT1) from |
| | | Brassica napus: effects of low temperature and |
| | | abscisic acid treatments on AAPT expression in |
| | | Arabidopsis plants and effects of over-expression |
| | | of BnAAPT1 in transgenic Arabidopsis.” Planta |
| | | 217(4): 547-558. |
| BnSIP1-1 | Tolerance to osmotic | Luo, J., et al. (2017). “BnSIP1-1, a Trihelix Family |
| Trihelix | stress and salt stress | Gene, Mediates Abiotic Stress Tolerance and ABA |
| Family Gene | | Signaling in Brassica napus.” Front Plant Sci 8: 44. |
| BnGLIP1 | Resistance to | Ding, L.N., et al. (2020). “Arabidopsis GDSL1 |
| | Sclerotinia sclerotiorum | overexpression enhances rapeseed Sclerotinia |
| | | sclerotiorum resistance and the functional |
| | | identification of its homolog in Brassica napus.” |
| | | Plant Biotechnol J 18(5): 1255-1270. |
| BnLEA (B. | Resistance to drought | Park, B.J., et al. (2005). Genetic improvement of |
| napus group 3 | and salt stress | Chinese cabbage for salt anddrought tolerance by |
| late | | constitutive expressionof a B. napusLEA gene. |
| embryogenesis | | Plant Science 169: 553-558. |
| abundant gene | | |
| BnPIP1 (B. | Drought resistance | Yu, Q., et al. (2005). Sense and antisense |
| napus plasma | | expression of plasma membrane aquaporin BnPIP1 |
| membrane | | from Brassica napus in tobacco and its effects on |
| aquaporin | | plant drought resistance. Plant Science 169: |
| | | 647-656. |
| BnLEA 4-1 | Drought resistance | Dalal, M., et al. (2019). Abiotic stress and |
| | | ABA-inducible Group 4 LEA from Brassica napus |
| | | plays a key role in salt and drought tolerance. |
| | | Journal of Biotechnology 139: 137-145. |
| BnCIPK6 | Salt resistance, low | Chen, L., et al. (2012) The Brassica napus |
| (CBL-interact | phosphorous tolerance | Calcineurin B-Like 1/CBL-interacting protein |
| ing protein | | kinase 6 (CBL1/CIPK6) component is involved in |
| kinase 6) | | the plant response to abiotic stress and ABA. |
| BnCIPK6M | | Journal of Experimental Botany 63: 6211-6222. |
| (CIPK6 | | |
| phosphomimic | | |
| form) | | |
| AINTEGUM | High yield | Kuluev, B.R., et al. (2013). “[Morphological |
| ENTA (ANT) | | features of transgenic tobacco plants expressing the |
| gene | | AINTEGUMENTA gene of rape under control of |
| | | the Dahlia mosaic virus promoter].” Ontogenez |
| | | 44(2): 110-114. |
| BnCOR25 | Cold resistance | Chen, L., et al. (2011). “A novel cold-regulated |
| | | gene, COR25, of Brassica napus is involved in |
| | | plant response and tolerance to cold stress.” Plant |
| | | Cell Rep 30(4): 463-471. |
| BnVQ7 | Disease resistance | Zou, Z., et al. (2020). “Genome-Wide Identification |
| (BnMKS1) | | and Analysis of VQ Motif-containing Gene Family |
| | | in Brassica napus and Functional Characterization |
| | | of BnMKS1 in Response to Leptosphaeria |
| | | maculans.” Phytopathology. |
| b-ketoacyl-A | To improve quality | Gupta, M., et al. (2012). “Transcriptional activation |
| CP synthase | | of Brassica napus beta-ketoacyl-ACP synthase II |
| II (KASII) | | with an engineered zinc finger protein transcription |
| | | factor.” Plant Biotechnol J 10(7): 783-791. |
| BnLEA3, | Drought resistance | Liang, Y., et al. (2019). “Drought-responsive genes, |
| BnVOC | | late embryogenesis abundant group3 (LEA3) and |
| | | vicinal oxygen chelate, function in lipid |
| | | accumulation in Brassica napus and Arabidopsis |
| | | mainly via enhancing photosynthetic efficiency and |
| | | reducing ROS.” Plant Biotechnol J 17(11): |
| | | 2123-2142. |
| BnaA3.MYB28 | To improve quality | Liu, S., et al. (2020). “Dissection of genetic |
| | | architecture for glucosinolate accumulations in |
| | | leaves and seeds of Brassica napus by genome-wide |
| | | association study.” Plant Biotechnol J 18(6): |
| | | 1472-1484. |
| BnaA9.CYP78A9 | To increase yield | Shi, L., et al. (2019). “A CACTA-like transposable |
| P450 | | element in the upstream region of |
| monooxygenase | | BnaA9.CYP78A9 acts as an enhancer to increase |
| | | silique length and seed weight in rapeseed.” Plant J |
| | | 98(3): 524-539. |
| BnHO-1 | Tolerance to Hg | Shen, Q., et al. (2011). “Expression of a Brassica |
| | pollution | napus heme oxygenase confers plant tolerance to |
| | | mercury toxicity.” Plant Cell Environ 34(5): |
| | | 752-763. |
| Al-activated | Tolerance to aluminum | Ligaba, A., et al. (2006). “The BnALMT1 and |
| malate | toxicity | BnALMT2 genes from rape encode |
| transporter) | | aluminum-activated malate transporters that |
| | | enhance the aluminum resistance of plant cells.” |
| | | Plant Physiol 142(3): 1294-1303. |
| SUPPRESSOR | To increase pod seed | Li, S., et al. (2015). “BnaC9.SMG7b Functions as a |
| WITH | number | Positive Regulator of the Number of Seeds per |
| MORPHOGENETIC | | Silique in Brassica napus by Regulating the |
| EFFECTS ON | | Formation of Functional Female Gametophytes.” |
| GENITALIA | | Plant Physiol 169(4): 2744-2760. |
| 7 | | |
| BnaA03.MPK6, | Resistance to | Wang, Z., et al. (2020). “BnaMPK6 is a |
| mitogen-activated | Sclerotinia sclerotiorum | determinant of quantitative disease resistance |
| protein kinases | | against Sclerotinia sclerotiorum in oilseed rape.” |
| | | Plant Sci 291: 110362. |
| PHT1 | To improve phosphate | Ren, F., et al. (2014). “A Brassica napus PHT1 |
| phosphate | uptake | phosphate transporter, BnPht1; 4, promotes |
| transporter, | | phosphate uptake and affects roots architecture of |
| BnPht1; 4 | | transgenic Arabidopsis.” Plant Mol Biol 86(6): |
| | | 595-607. |
| proline-rich, | To increase yield | Haffani, Y. Z., et al. (2006). “Altered Expression of |
| extensin-like | | PERK Receptor Kinases in Arabidopsis Leads to |
| receptor | | Changes in Growth and Floral Organ Formation.” |
| kinase | | Plant Signal Behav 1(5): 251-260. |
| (PERK) | | |
| BnSIP1-1 | Tolerance to osmotic | Luo, J., et al. (2017). “BnSIP1-1, a Trihelix Family |
| | and salt stress in | Gene, Mediates Abiotic Stress Tolerance and ABA |
| | germination stage | Signaling in Brassica napus.” Front Plant Sci 8: 44. |
| LTP2 | To increase trichome | Peng, D., et al. (2018). “Enhancing freezing |
| | density, change | tolerance of Brassica napus L. by overexpression of |
| | secondary metabolite | a stearoyl-acyl carrier protein desaturase gene |
| | concentration | (SAD) from Sapium sebiferum (L.) Roxb.” Plant |
| | | Sci 272: 32-41. |
| BnPGIP2 | Resistance to | Wang, Z., et al. (2018). “Overexpression of |
| | Sclerotinia sclerotiorum | OsPGIP2 confers Sclerotinia sclerotiorum |
| | | resistance in Brassica napus through increased |
| | | activation of defense mechanisms.” J Exp Bot |
| | | 69(12): 3141-3155. |
| BnLAS | To increase plant | Yang, M., et al. (2011). “Overexpression of the |
| | drought tolerance | Brassica napus BnLAS gene in Arabidopsis affects |
| | | plant development and increases drought |
| | | tolerance.” Plant Cell Rep 30(3): 373-388. |
| CBF/ | To improve | Savitch, L.V., et al. (2005). “The effect of |
| dreb1type | photosynthetic capacity | overexpression of two Brassica CBF/DREB1-like |
| transcription | and freezing tolerance | transcription factors on photosynthetic capacity and |
| factor | | freezing tolerance in Brassica napus.” Plant Cell |
| | | Physiol 46(9): 1525-1539. |
| BnWRKY33 | To enhance resistance | Wang, Z., et al. (2014). “Overexpression of |
| | to Sclerotinia | BnWRKY33 in oilseed rape enhances resistance to |
| | sclerotiorum | Sclerotinia sclerotiorum.” Mol Plant Pathol 15(7): |
| | | 677-689. |
| BnSCE3 | To inhibit sinapine | Clauss, K., et al. (2011). “Overexpression of |
| | accumulation | sinapine esterase BnSCE3 in oilseed rape seeds |
| | | triggers global changes in seed metabolism.” Plant |
| | | Physiol 155(3): 1127-1145. |
| MYB43 | Positively regulates | Jiang, J., et al. (2020). “MYB43 in Oilseed Rape |
| | vascular lignification, | (Brassica napus) Positively Regulates Vascular |
| | plant morphology and | Lignification, Plant Morphology and Yield |
| | Yield potential but | Potential but Negatively Affects Resistance to |
| | negatively affects | Sclerotinia sclerotiorum.” Genes (Basel) 11(5). |
| | resistance to Sclerotinia | |
| | sclerotiorum | |
| PAT15 | To increase branch and | Peng, D., et al. (2018). “Increasing branch and seed |
| | seed yield | yield through heterologous expression of the novel |
| | | rice S-acyl transferase gene OsPAT15 in Brassica |
| | | napus L.” Breed Sci 68(3): 326-335. |
| BnNRT2.2 | To increase nitrate | Faure-Rabasse, S., et al. (2002). “Effects of nitrate |
| | influx rates | pulses on BnNRT1 and BnNRT2 genes: mRNA |
| | | levels and nitrate influx rates in relation to the |
| | | duration of N deprivation in Brassica napus L.” J |
| | | Exp Bot 53(375): 1711-1721. |

Table D lists important functional genes in some horticulture crops. Methods in the present invention can be used to edit such genes and create new genes by designing new combinations of different gene elements or different protein domain.

TABLE D
Important functional genes in horticulture crops

| Crop | Gene name | Application | Reference |
|---|---|---|---|
| Apple | MdATG18a | Thermo tolerance | Huo, L., et al. (2020). “MdATG18a |
| | | | overexpression improves basal |
| | | | thermotolerance in transgenic apple by |
| | | | decreasing damage to chloroplasts.” Hortic |
| | | | Res 7: 21. |
| Apple | MdSPL13 | Salt stressresistance | Ma, Y., et al. (2021). “The miR156/SPL |
| | | | module regulates apple salt stress tolerance |
| | | | by activating MdWRKY100 expression.” |
| | | | Plant Biotechnol J 19(2): 311-323. |
| Apple | MdDREB76 | Drought tolerance | Sharma, V., et al. (2019). “An apple |
| | | and salt resistance | transcription factor, MdDREB76, confers salt |
| | | | and drought tolerance in transgenic tobacco |
| | | | by activating the expression of |
| | | | stress-responsive genes.” Plant Cell Rep |
| | | | 38(2): 221-241. |
| Apple | MdANK2B | Salt tolerance and | Zhang, F. J., et al. (2021). “The ankyrin |
| | | ABA sensitivity | repeat-containing protein MdANK2B |
| | | | regulates salt tolerance and ABA sensitivity |
| | | | in Malus domestica.” Plant Cell Rep 40(2): |
| | | | 405-419. |
| Apple | MdbHLH3 | Quality, | Yu, J.Q., et al. (2021). “The apple bHLH |
| | | carbohydrate and | transcription factor MdbHLH3 functions in |
| | | malic acid | determining the fruit carbohydrates and |
| | | | malate.” Plant Biotechnol J 19(2): 285-299. |
| Apple | MdIAA24 | Drought tolerance | Huang, D., et al. (2021). “Overexpression of |
| | | | MdIAA24 improves apple drought resistance |
| | | | by positively regulating strigolactone |
| | | | biosynthesis and mycorrhization.” Tree |
| | | | Physiol 41(1): 134-146. |
| Apple | MdNAC42 | Anthocyanin | Zhang, S., et al. (2020). “A novel NAC |
| | | | transcription factor, MdNAC42, regulates |
| | | | anthocyanin accumulation in red-fleshed |
| | | | apple by interacting with MdMYB10.” Tree |
| | | | Physiol 40(3): 413-423. |
| Apple | MdHAL3 | Salt tolerance | Yang, S., et al. (2020). “MdHAL3, a |
| | | | 4′-phosphopantothenoylcysteine |
| | | | decarboxylase, is involved in the salt |
| | | | tolerance of autotetraploid apple.” Plant Cell |
| | | | Rep 39(11): 1479-1491. |
| Apple | MdWRKY11- | Copper tolerance | Shi, K., et al. (2020). “MdWRKY11 improves |
| | MdHMA5 | | copper tolerance by directly promoting the |
| | | | expression of the copper transporter gene |
| | | | MdHMA5.” Hortic Res 7: 105. |
| Apple | MdATG9 | Nitrogen stress | Huo, L., et al. (2020). “The Apple |
| | | | Autophagy-Related Gene MdATG9 Confers |
| | | | Tolerance to Low Nitrogen in Transgenic |
| | | | Apple Callus.” Front Plant Sci 11: 423. |
| Apple | MdATG10 | Salt tolerance | Huo, L., et al. (2020). “Increased autophagic |
| | | | activity in roots caused by overexpression of |
| | | | the autophagy-related gene MdATG10 in |
| | | | apple enhances salt tolerance.” Plant Sci 294: |
| | | | 110444. |
| Apple | MdTYDC | alleviate replant | Gao, T., et al. (2020). “Exogenous dopamine |
| | | disease | and overexpression of the dopamine synthase |
| | | | gene MdTYDC alleviated apple replant |
| | | | disease.” Tree Physiol. |
| Apple | MdWRKY26/ | Salt tolerance and | Dong, Q., et al. (2020). “MdWRKY30, a |
| | 28/30 | osmotic stress | group Ila WRKY gene from apple, confers |
| | | | tolerance to salinity and osmotic stresses in |
| | | | transgenic apple callus and Arabidopsis |
| | | | seedlings.” Plant Sci 299: 110611. |
| Apple | MdCERK1-2 | resistance to | Chen, Q., et al. (2020). “Overexpression of an |
| | | pathogenic fungus | apple LysM-containing protein gene, |
| | | | MdCERK1-2, confers improved resistance to |
| | | | the pathogenic fungus, Alternaria alternata, in |
| | | | Nicotiana benthamiana.” BMC Plant Biol |
| | | | 20(1): 146. |
| Apple | MdBAK1 | Growth and | Zheng, L., et al. (2019). “Transcriptome |
| | | development | Analysis Reveals New Insights into |
| | | | MdBAK1-Mediated Plant Growth in Malus |
| | | | domestica.” J Agric Food Chem 67(35): |
| | | | 9757-9771. |
| Apple | MdWRKY100 | Resistance to | Zhang, F., et al. (2019). “MdWRKY100 |
| | | Colletotrichum | encodes a group I WRKY transcription factor |
| | | gloeosporioides | in Malus domestica that positively regulates |
| | | infection | resistance to Colletotrichum gloeosporioides |
| | | | infection.” Plant Sci 286: 68-77. |
| Apple | Ma10 | Acidity | Ma, B., et al. (2019). “A Ma10 gene encoding |
| | | | P-type ATPase is involved in fruit organic |
| | | | acid accumulation in apple.” Plant Biotechnol |
| | | | J 17(3): 674-686. |
| Apple | MdNAC1 | Drought resistance | Jia, D., et al. (2019). “An apple (Malus |
| | | | domestica) NAC transcription factor enhances |
| | | | drought tolerance in transgenic apple plants.” |
| | | | Plant Physiol Biochem 139: 504-512. |
| Apple | MdIAA9 | Osmotic stress | Huang, D., et al. (2019). “Overexpression of |
| | | | MdIAA9 confers high tolerance to osmotic |
| | | | stress in transgenic tobacco.” PeerJ 7: e7935. |
| Apple | MdABCG28 | Stem growth | Feng, Y., et al. (2019). “Genome-Wide |
| | | | Identification and Characterization of ABC |
| | | | Transporters in Nine Rosaceae Species |
| | | | Identifying MdABCG28 as a Possible |
| | | | Cytokinin Transporter linked to Dwarfing.” |
| | | | Int J Mol Sci 20(22). |
| Apple | MdWRKY9 | Dwarfing | Zheng, X., et al. (2018). “MdWRKY9 |
| | | | overexpression confers intensive dwarfing in |
| | | | the M26 rootstock of apple by directly |
| | | | inhibiting brassinosteroid synthetase |
| | | | MdDWF4 expression.” New Phytol 217(3): |
| | | | 1086-1098. |
| Apple | MdERF1B | Anthocyanin | Zhang, J., et al. (2018). “The ethylene |
| | | | response factor MdERF1B regulates |
| | | | anthocyanin and proanthocyanidin |
| | | | biosynthesis in apple.” Plant Mol Biol 98(3): |
| | | | 205-218. |
| Apple | MdATG18a | Drought tolerance | Sun, X., et al. (2018). “Improvement of |
| | | | drought tolerance by overexpressing |
| | | | MdATG18a is mediated by modified |
| | | | antioxidant system and activated autophagy in |
| | | | transgenic apple.” Plant Biotechnol J 16(2): |
| | | | 545-557. |
| Apple | MdWRKY79- | Fungus resistance | Meng, D., et al. (2018). “Sorbitol Modulates |
| | MdNLR16 | | Resistance to Alternaria altemata by |
| | | | Regulating the Expression of an NLR |
| | | | Resistance Gene in Apple.” Plant Cell 30(7): |
| | | | 1562-1581. |
| Apple | MdSAP15 | Drought tolerance | Dong, Q., et al. (2018). “Genome-Wide |
| | | | Analysis and Cloning of the Apple |
| | | | Stress-Associated Protein Gene Family |
| | | | Reveals MdSAP15, Which Confers Tolerance |
| | | | to Drought and Osmotic Stresses in |
| | | | Transgenic Arabidopsis.” Int J Mol Sci 19(9). |
| Pepper | CaNAC46 | Salt and drought | Ma, J., et al. (2021). “The NAC-type |
| | | tolerance | transcription factor CaNAC46 regulates the |
| | | | salt and drought tolerance of transgenic |
| | | | Arabidopsis thaliana.” BMC Plant Biol 21(1): |
| | | | 11. |
| Pepper | CaSBP08 | Phytophthora | Zhang, H.X., et al. (2020). “Identification of |
| | | capsici resistance | Pepper CaSBP08 Gene in Defense Response |
| | | | Against Phytophthora capsici Infection.” |
| | | | Front Plant Sci 11: 183. |
| Pepper | CaMLO6 | Thermo resistance | Yang, S., et al. (2020). “Pepper CaMLO6 |
| | | | Negatively Regulates Ralstonia solanacearum |
| | | | Resistance and Positively Regulates High |
| | | | Temperature and High Humidity Responses.” |
| | | | Plant Cell Physiol 61(7): 1223-1238. |
| Pepper | CaCBL1 | Ralstonia | Shen, L., et al. (2020). “CaCBL1 Acts as a |
| | | solanacearum | Positive Regulator in Pepper Response to |
| | | resistance | Ralstonia solanacearum.” Mol Plant Microbe |
| | | | Interact 33(7): 945-957. |
| Pepper | CaNHL4 | Pathogenic bacteria | Liu, C., et al. (2020). “Genome-wide analysis |
| | | resistance | of NDR1/HIN1-like genes in pepper |
| | | | (Capsicum annuum L.) and functional |
| | | | characterization of CaNHL4 under biotic and |
| | | | abiotic stresses.” Hortic Res 7: 93. |
| Pepper | CaHsp26.5 | Virus defense | Foong, S. L. et al. (2020). “Capsicum annum |
| | | | Hsp26.5 promotes defense responses against |
| | | | RNA viruses via ATAF2 but is hijacked as a |
| | | | chaperone for tobamovirus movement |
| | | | protein.” J Exp Bot 71(19): 6142-6158. |
| Pepper | CaChiVI2 | Thermo tolerance | Ali, M., et al. (2020). “The CaChiVI2 Gene of |
| | | and disease | Capsicum annuum L. Confers Resistance |
| | | resistance | Against Heat Stress and Infection of |
| | | | Phytophthora capsici.” Front Plant Sci 11: |
| | | | 219. |
| Pepper | CaLRR-RLK1 | Ralstonia | Mou, S., et al. (2019). “CaLRR-RLK1, a |
| | | solanacearum | novel RD receptor-like kinase from Capsicum |
| | | resistance | annuum and transcriptionally activated by |
| | | | CaHDZ27, act as positive regulator in |
| | | | Ralstonia solanacearum resistance.” BMC |
| | | | Plant Biol 19(1): 28. |
| Pepper | CaHSP16.4 | Thermo and | Huang, L.J., et al. (2019). “CaHSP16.4, a |
| | | drought tolerance | small heat shock protein gene in pepper, is |
| | | | involved in heat and drought tolerance.” |
| | | | Protoplasma 256(1): 39-51. |
| Pepper | CaWRKY41 | Ralstonia | Dang, F., et al. (2019). “A feedback loop |
| | | solanacearum | between CaWRKY41 and H2O2 coordinates |
| | | resistance | the response to Ralstonia solanacearum and |
| | | | excess cadmium in pepper.” J Exp Bot 70(5): |
| | | | 1581-1595. |
| Pepper | CaC3H14 | Ralstonia | Qiu, A., et al. (2018). “CaC3H14 encoding a |
| | | solanacearum | tandem CCCH zinc finger protein is directly |
| | | resistance | targeted by CaWRKY40 and positively |
| | | | regulates the response of pepper to |
| | | | inoculation by Ralstonia solanacearum.” Mol |
| | | | Plant Pathol 19(10): 2221-2235. |
| Pepper | CaWRKY22 | Ralstonia | Hussain, A., et al. (2018). “CaWRKY22 Acts |
| | | solanacearum | as a Positive Regulator in Pepper Response to |
| | | resistance | Ralstonia Solanacearum by Constituting |
| | | | Networks with CaWRKY6, CaWRKY27, |
| | | | CaWRKY40, and CaWRKY58.” Int J Mol Sci |
| | | | 19(5). |
| Pepper | CaHSL1 | Thermo tolerance | Guan, D., et al. (2018). “CaHSL1 Acts as a |
| | | | Positive Regulator of Pepper |
| | | | Thermotolerance Under High Humidity and Is |
| | | | Transcriptionally Modulated by |
| | | | CaWRKY40.” Front Plant Sci 9: 1802. |
| Pepper | HsfB2a | Thermo tolerance | Ashraf, M.F., et al. (2018). “Capsicum |
| | | and Ralstonia | annuum HsfB2a Positively Regulates the |
| | | solanacearum | Response to Ralstonia solanacearum Infection |
| | | resistance | or High Temperature and High Humidity |
| | | | Forming Transcriptional Cascade with |
| | | | CaWRKY6 and CaWRKY40.” Plant Cell |
| | | | Physiol 59(12): 2608-2623. |
| Pepper | CanPI7 | Insect resistance | Tanpure, R.S., et al. (2017). “Improved |
| | | | tolerance against Helicoverpa armigera in |
| | | | transgenic tomato over-expressing |
| | | | multi-domain proteinase inhibitor gene from |
| | | | Capsicum annuum.” Physiol Mol Biol Plants |
| | | | 23(3): 597-604. |
| Pepper | CaRDR1 | Resistance to | Qin, L., et al. (2017). “CaRDR1, an |
| | | TMV | RNA-Dependent RNA Polymerase Plays a |
| | | | Positive Role in Pepper Resistance against |
| | | | TMV.” Front Plant Sci 8: 1068. |
| Pepper | CaLRR51 | Ralstonia | Cheng, W., et al. (2017). “A novel |
| | | solanacearum | leucine-rich repeat protein, CaLRR51, acts as |
| | | resistance | a positive regulator in the response of pepper |
| | | | to Ralstonia solanacearum infection.” Mol |
| | | | Plant Pathol 18(8): 1089-1100. |
| Pepper | CabZIP63 | High temperature | Shen, L., et al. (2016). “Pepper CabZIP63 |
| | | tolerance | acts as a positive regulator during Ralstonia |
| | | | solanacearum or high temperature-high |
| | | | humidity challenge in a positive feedback |
| | | | loop with CaWRKY40.” J Exp Bot 67(8): |
| | | | 2439-2451. |
| Pepper | CaWRKY6 | Ralstonia | Cai, H., et al. (2015). “CaWRKY6 |
| | | solanacearum | transcriptionally activates CaWRKY40, |
| | | resistance | regulates Ralstonia solanacearum resistance, |
| | | | and confers high-temperature and |
| | | | high-humidity tolerance in pepper.” J Exp Bot |
| | | | 66(11): 3163-3174. |
| Pepper | CaDSR6 | Drought and salt | Kim, E.Y., et al. (2014). “Overexpression of |
| | | tolerance | CaDSR6 increases tolerance to drought and |
| | | | salt stresses in transgenic Arabidopsis plants.” |
| | | | Gene 552(1): 146-154. |
| Pepper | CaWRKY27 | Ralstonia | Dang, F., et al. (2014). “Overexpression of |
| | | solanacearum | CaWRKY27, a subgroup lie WRKY |
| | | resistance | transcription factor of Capsicum annuum, |
| | | | positively regulates tobacco resistance to |
| | | | Ralstonia solanacearum infection.” Physiol |
| | | | Plant 150(3): 397-411. |
| Pepper | CaAMP1 | Fungus resistance | Lee, S.C., et al. (2008). “Involvement of the |
| | | | pepper antimicrobial protein CaAMP1 gene in |
| | | | broad spectrum disease resistance.” Plant |
| | | | Physiol 148(2): 1004-1020. |
| Pepper | CaPMEI1 | Fungus resistance | An, S.H., et al. (2008). “Pepper pectin |
| | | | methylesterase inhibitor protein CaPMEI1 is |
| | | | required for antifungal activity, basal disease |
| | | | resistance and abiotic stress tolerance.” Planta |
| | | | 228(1): 61-78. |
| Grape | VvChi5, | Fungus resistance, | Zheng, T., et al. (2020). “Chitinase family |
| | VvChi17, | fruit storage | genes in grape differentially expressed in a |
| | VvChi22, | | manner specific to fruit species in response to |
| | VvChi26 | | Botrytis cinerea.” Mol Biol Rep 47(10): |
| | VvChi31 | | 7349-7363. |
| Albizia | IpDGAT2 | Lipid content | Fan, R., et al. (2021). “Characterization of |
| julibrissin | | | diacylglycerol acyltransferase 2 from Idesia |
| | | | polycarpa and function analysis.” Chem Phys |
| | | | Lipids 234: 105023. |
| Grape | VvBAP1 | Thermo stress | Ye, Q., et al. (2020). “VvBAPI1 a Grape C2 |
| | | tolerance | Domain Protein, Plays a Positive Regulatory |
| | | | Role Under Heat Stress.” Front Plant Sci 11: |
| | | | 544374. |
| Grape | VvKCS | Salt tolerance | Yang, Z., et al. (2020). “Overexpression of |
| | | | beta-Ketoacyl-CoA Synthase From Vitis |
| | | | vinifera L. Improves Salt Tolerance in |
| | | | Arabidopsis thaliana.” Front Plant Sci 11: |
| | | | 564385. |
| Grape | VvCKX5 | To reduce the | Moriyama, A., et al. (2020). “Crosstalk |
| | | number of flower | Pathway between Trehalose Metabolism and |
| | | buds per | Cytokinin Degradation for the Determination |
| | | inflorescence | of the Number of Berries per Bunch in |
| | | | Grapes.” Cells 9(11). |
| Grape | VvCEB1opt | Drought tolerance | Lim, S.D., et al. (2020). “Plant tissue |
| | | | succulence engineering improves water-use |
| | | | efficiency, water-deficit stress attenuation and |
| | | | salinity tolerance in Arabidopsis.” Plant J |
| | | | 103(3): 1049-1072. |
| Grape | VvERF1 | Botrytis cinerea | Dong, T., et al. (2020). “The Effect of |
| | | resistance | Ethylene on the Color Change and Resistance |
| | | | to Botrytis cinerea Infection in ‘Kyoho’ Grape |
| | | | Fruits.” Foods 9(7). |
| Grape | VvSUC11, | To enhance drought | Cai, Y., et al. (2020). “Expression of Sucrose |
| | VvSUC27 | resistance | Transporters from Vitis vinifera Confer High |
| | | | Yield and Enhances Drought Resistance in |
| | | | Arabidopsis.” Int J Mol Sci 21(7). |
| Grape | VvWRKY30 | improve salt stress | Zhu, D., et al. (2019). “VvWRKY30, a grape |
| | | tolerance | WRKY transcription factor, plays a positive |
| | | | regulatory role under salinity stress.” Plant |
| | | | Sci 280: 132-142. |
| Grape | VvSWEET10 | increase sugar | Zhang, Z., et al. (2019). “VvSWEET10 |
| | | accumulation | Mediates Sugar Accumulation in Grapes.” |
| | | | Genes (Basel) 10(4). |
| Grape | VvDOF3 | enhance powdery | Yu, Y.H., et al. (2019). “Grape (Vitis |
| | | mildew resistance | vinifera) VvDOF3 functions as a transcription |
| | | | activator and enhances powdery mildew |
| | | | resistance.” Plant Physiol Biochem 143: |
| | | | 183-189. |
| Grape | VvTIFY9 | Closely relates to | Yu, Y., et al. (2019). “Functional |
| | | sa-mediated | Characterization of Resistance to Powdery |
| | | powdery mildew | Mildew of VvTIFY9 from Vitis vinifera.” Int |
| | | resistance in grapes | J Mol Sci 20(17). |
| Grape | VdMYB1 | Positively regulates | Yu, Y., et al. (2019). “The grapevine |
| | | defensive response | R2R3-type MYB transcription factor |
| | | and increases | VdMYB1 positively regulates defense |
| | | resveratrol content | responses by activating the stilbene synthase |
| | | in leaves | gene 2 (VdSTS2).” BMC Plant Biol 19(1): |
| | | | 478. |
| Grape | VaERF092 | improve cold | Sun, X., et al. (2019). “The ethylene response |
| | VaWRKY33 | tolerance | factor VaERF092 from Amur grape regulates |
| | | | the transcription factor VaWRKY33, |
| | | | improving cold tolerance.” Plant J 99(5): |
| | | | 988-1002. |
| Grape | VqSTS6 | enhance ethylene | Liu, M., et al. (2019). “Expression of stilbene |
| | | compounds | synthase VqSTS6 from wild Chinese Vitis |
| | | accumulation and | quinquangularis in grapevine enhances |
| | | improve disease | resveratrol production and powdery mildew |
| | | resistance | resistance.” Planta 250(6): 1997-2007. |
| Grape | VbDFR | To increase | Zhu, Y., et al. (2018). “Molecular Cloning |
| | | anthocyanin | and Functional Characterization of a |
| | | production in | Dihydroflavonol 4-Reductase from Vitis |
| | | flowers | bellula.” Molecules 23(4). |
| Grape | VvCEB1opt | To show larger | Lim, S.D., et al. (2018). “A Vitis vinifera |
| | | cells, organ size | basic helix-loop-helix transcription factor |
| | | and vegetative | enhances plant cell size, vegetative biomass |
| | | biomass | and reproductive yield.” Plant Biotechnol J. |
| Grape | VpSBP16 | To improve | Hou, H., et al. (2018). “Overexpression of a |
| | | tolerance to salt and | SBP-Box Gene (VpSBP16) from Chinese |
| | | drought stress | Wild Vitis Species in Arabidopsis Improves |
| | | | Salinity and Drought Stress Tolerance.” Int J |
| | | | Mol Sci 19(4). |
| Grape | VpTNL1 | Resistance to strong | Wen, Z., et al. (2017). “Constitutive |
| | | pathogenic bacteria | heterologous overexpression of a |
| | | pseudomonas | TIR-NB-ARC-LRR gene encoding a putative |
| | | syringae | disease resistance protein from wild Chinese |
| | | | Vitis pseudoreticulata in Arabidopsis and |
| | | | tobacco enhances resistance to |
| | | | phytopathogenic fungi and bacteria.” Plant |
| | | | Physiol Biochem 112: 346-361. |
| Grape | VpRH2 | Resistance to | Wang, L., et al. (2017). “RING-H2-type E3 |
| | | powdery mildew | gene VpRH2 from Vitis pseudoreticulata |
| | | | improves resistance to powdery mildew by |
| | | | interacting with VpGRP2A.” J Exp Bot 68(7): |
| | | | 1669-1687. |
| Grape | VvVHP1; 2 | To improve | Sun, T., et al. (2017). “VvVHP2; 2 Is |
| | | anthocyaninaccumu | Transcriptionally Activated by VvMYBA1 |
| | | lation | and Promotes Anthocyanin Accumulation of |
| | | | Grape Berry Skins via Glucose Signal.” Front |
| | | | Plant Sci 8: 1811. |
| Grape | VaPUB | Be able to have | Jiao, L., et al. (2017). “Overexpression of a |
| | | quick response to | stress-responsive U-box protein gene VaPUB |
| | | biotic and abiotic | affects the accumulation of resistance related |
| | | stress and | proteins in Vitis vinifera ‘Thompson |
| | | obviously affect | Seedless’.” Plant Physiol Biochem 112: |
| | | accumulation of | 53-63. |
| | | disease resistance | |
| | | related proteins | |
| Epimedium | EsMYB9 | To increase | Huang, W., et al. (2017). “Functional |
| | | anthocyanin and | Characterization of a Novel R2R3-MYB |
| | | flavonol content | Transcription Factor Modulating the |
| | | | Flavonoid Biosynthetic Pathway from |
| | | | Epimedium sagittatum.” Front Plant Sci 8: |
| | | | 1274. |
| Grape | VvSUC27 | To play an | Cai, Y., et al. (2017). “Overexpression of a |
| | | important role in | Grapevine Sucrose Transporter (VvSUC27) in |
| | | biotic and abiotic | Tobacco Improves Plant Growth Rate in the |
| | | stress response, | Presence of Sucrose In vitro.” Front Plant Sci |
| | | especially in the | 8: 1069. |
| | | presence of sucrose | |
| Grape | VqDUF642 | To promote plant | Xie, X. and Y. Wang (2016). “VqDUF642, a |
| | | growth, reduce | gene isolated from the Chinese grape Vitis |
| | | botrytis cinerea | quinquangularis, is involved in berry |
| | | sensibility and | development and pathogen resistance.” Planta |
| | | enhance resistance | 244(5): 1075-1094. |
| | | to erysipelas and | |
| | | Metarhizium | |
| | | anisopliae | |
| Grape | VaCPK20 | To make stress | Dubrovina, A.S., et al. (2015). “VaCPK20, a |
| | | response in | calcium-dependent protein kinase gene of |
| | | non-stress | wild grapevine Vitis amurensis Rupr., |
| | | conditions, | mediates cold and drought stress tolerance.” J |
| | | post-freezing and | Plant Physiol 185: 1-12. |
| | | drought stress | |
| Grape | VaCPK29 | Positively regulates | Aleynova, O.A., et al. (2015). “Regulation of |
| | VaCPK20 | factors take part in | resveratrol production in Vitis amurensis cell |
| | | the biosynthesis of | cultures by calcium-dependent protein |
| | | resveratrol | kinases.” Appi Biochem Biotechnol 175(3): |
| | | | 1460-1476. |
| Grape | VvABF2 | The overexpression | Nicolas, P., et al. (2014). “The basic leucine |
| | | strongly enhances | zipper transcription factor ABSCISIC ACID |
| | | the accumulation of | RESPONSE ELEMENT-BINDING |
| | | diphenylethene | FACTOR2 is an important transcriptional |
| | | (resveratrol) which | regulator of abscisic acid-dependent grape |
| | | is beneficial to | berry ripening processes.” Plant Physiol |
| | | plant defense and | 164(1): 365-383. |
| | | human healthy | |
| Grape | VvDRT100-L | To obtain | Fujimori, N., et al. (2014). “Plant |
| | | adaptability, | DNA-damage repair/toleration 100 protein |
| | | tolerance and DNA | repairs UV-B-induced DNA damage.” DNA |
| | | repairation to | Repair (Amst) 21: 171-176. |
| | | ultraviolet light | |
| | | stress | |
| Grape | VvWRKY1 | To enhance | Marchive, C., et al. (2013). “Over-expression |
| | | resistance to downy | of VvWRKY1 in grapevines induces |
| | | mildew in grapes | expression of jasmonic acid pathway-related |
| | | | genes and confers higher tolerance to the |
| | | | downy mildew.” PLoS One 8(1): e54185. |
| Grape | VvIAA19 | To hasten growth | Kohno, M., et al. (2012). |
| | | speed, including | “Auxin-nonresponsive grape Aux/IAA19 is a |
| | | root elongation and | positive regulator of plant growth.” Mol Biol |
| | | flower | Rep 39(2): 911-917. |
| | | transformation | |
| Grape | VvCBF2 | Tolerance to cold, | Kobayashi, M., et al. (2012). |
| | VvZFPL | drought and salt | “Characterization of grape C-repeat-binding |
| | | stress | factor 2 and B-box-type zinc finger protein in |
| | | | transgenic Arabidopsis plants under stress |
| | | | conditions.” Mol Biol Rep 39(8): 7933-7939. |
| Grape | VvMYB5b | Anthocyanin and | Deluc, L., et al. (2008). “The transcription |
| | | procyanidine | factor VvMYB5b contributes to the regulation |
| | | derivate | of anthocyanin and proanthocyanidin |
| | | accumulation | biosynthesis in developing grape berries.” |
| | | | Plant Physiol 147(4): 2041-2053. |
| Grape | VvWRKY2 | Resistance to | Mzid, R., et al. (2007). “Overexpression of |
| | | fungal pathogens | VvWRKY2 in tobacco enhances broad |
| | | | resistance to necrotrophic fungal pathogens.” |
| | | | Physiol Plant 131(3): 434-447. |
| Grape | VvWRKY1 | Resistance to | Marchive, C., et al. (2007). “Isolation and |
| | | fungal pathogens | characterization of a Vitis vinifera |
| | | | transcription factor, VvWRKY1, and its |
| | | | effect on responses to fungal pathogens in |
| | | | transgenic tobacco plants.” J Exp Bot 58(8): |
| | | | 1999-2010. |
| Grape | VvMYBSa | To increase the | Deluc, L., et al. (2006). “Characterization of a |
| | | biosynthesis of | grapevine R2R3-MYB transcription factor |
| | | condensed tannins | that regulates the phenylpropanoid pathway.” |
| | | and change xylogen | Plant Physiol 140(2): 499-511. |
| | | metabolism | |
| Eggplant | SmMYB44 | Ralstonia | Qiu, Z., et al. (2019). “The eggplant |
| | | solanacearum | transcription factor myb44 enhances |
| | | resistance | resistance to bacterial wilt by activating the |
| | | | expression of spermidine synthase”. Journal |
| | | | of Experimental Botany(19), 19. |
| Eggplant | SmMYB1 | Anthocyanin | Zhang, Y., et al. (2014). “Anthocyanin |
| | | accumulation | accumulation and molecular analysis of |
| | | | anthocyanin biosynthesis-associated genes in |
| | | | eggplant (Solanum melongena L.).” Journal |
| | | | of Agricultural & Food Chemistry 62(13): |
| | | | 2906. |
| Eggplant | SmCBFs | Anthocyanin | Zhou, L., et al. (2019). “CBFs Function in |
| | SmMYB113 | accumulation | Anthocyanin Biosynthesis by Interacting with |
| | | | MYB113 in Eggplant (Solanum melongena |
| | | | L.).” Plant and Cell Physiology(2): 2. |
| Eggplant | SmMLO1 | Powdery mildew | Bracuto, V., et al. (2017). “Functional |
| | | susceptibility genes | characterization of the powdery mildew |
| | | | susceptibility gene SmMLO1 in eggplant |
| | | | (Solanum melongena L.).” Transgenic |
| | | | Research 26(3): 1-8. |
| Chinese | BrANT-1 | To regulate organ | Ding, Q., et al. (2018). “Ectopic expression of |
| cabbage | | size of Chinese | a Brassica rapa AINTEGUMENTA gene |
| | | cabbage | (BrANT-1) increases organ size and stomatal |
| | | | density in Arabidopsis.” Sci Rep 8(1): |
| | | | 10528.8(1):10528-. |
| Chinese | Brnym1 | To keep green | Wang, N., et al. (2020). “Defect in Brnym1, a |
| cabbage | | phenotype of leaves | magnesium-dechelatase protein, causes a |
| | | | stay-green phenotype in an EMS-mutagenized |
| | | | Chinese cabbage (Brassica campestris L. ssp. |
| | | | pekinensis) line.” Hortic Res 7(1): 8. |
| Chinese | BrARGOS | To regulate organ | Wang, B., et al. (2010). “Ectopic expression |
| cabbage | | size of Chinese | of a Chinese cabbage BrARGOS gene in |
| | | cabbage | Arabidopsis increases organ size.” Transgenic |
| | | | Res 19(3): 461-472. |
| Chinese | Bra040093 | Relates to petal | Peng, S., et al. (2019). “Mutation of ACX1, a |
| cabbage | | development in | Jasmonic Acid Biosynthetic Enzyme, Leads |
| | | Chinese cabbage | to Petal Degeneration in Chinese Cabbage |
| | | | (Brassica campestris ssp. pekinensis).” Int J |
| | | | Mol Sci 20(9). |
| Chinese | BrpSPL9-2 | Early-maturing | Wang, Y., et al. (2014). “BrpSPL9 (Brassica |
| cabbage | | improvement | rapa ssp. pekinensis SPL9) controls the |
| | | | earliness of heading time in Chinese |
| | | | cabbage.” Plant Biotechnol J 12(3): 312-321. |
| Chinese | BrWRKY12 | Resistance to carrot | Kim, H.S., et al. (2014). “Overexpression of |
| cabbage | | bacterial blight | the Brassica rapa transcription factor |
| | | | WRKY12 results in reduced soft rot |
| | | | symptoms caused by Pectobacterium |
| | | | carotovorum in Arabidopsis and Chinese |
| | | | cabbage.” Plant Biol (Stuttg) 16(5): 973-981. |
| Radish | RsPAP2 | Anthocyanin | Fan, L., et al. (2020). “A genome-wide |
| | | accumulation | association study uncovers a critical role of |
| | | | the RsPAP2 gene in red-skinned Raphanus |
| | | | sativus L.” Hortic Res 7: 164. |
| Radish | RsCPA31 | Salt stress tolerance | Wang, Y., et al. (2020). “Genome-Wide |
| | (RsNHX1) | | Identification and Functional Characterization |
| | | | of the Cation Proton Antiporter (CPA) Family |
| | | | Related to Salt Stress Response in Radish |
| | | | (Raphanus sativus L.).” Int J Mol Sci 21(21). |
| Radish | RsOFP2.3 | To regulate | Wang, Y., et al. (2020). “Characterization of |
| | | tuberous root shape | the OFP Gene Family and its Putative |
| | | | Involvement of Tuberous Root Shape in |
| | | | Radish.” Int J Mol Sci 21(4). |
| Pepper | CaASR1 | Ralstonia | Huang, J., et al. (2020). “CaASR1 promotes |
| | | solanacearum | salicylic acid- but represses jasmonic |
| | | resistance | acid-dependent signaling to enhance the |
| | | | resistance of Capsicum annuum to bacterial |
| | | | wilt by modulating CabZIP63.” J Exp Bot |
| | | | 71(20): 6538-6554. |
| Pepper | CaChiVI2 | Thermo and | Ali, M., et al. (2020). “The CaChiVI2 Gene of |
| | | drought tolerance | Capsicum annuum L. Confers Resistance |
| | | | Against Heat Stress and Infection of |
| | | | Phytophthora capsici.” Front Plant Sci 11: |
| | | | 219. |
| Pepper | CaNACO35 | Tolerance to abiotic | Zhang, H., et al. (2020). “Molecular and |
| | | stresses | Functional Characterization of CaNAC035, |
| | | | an NAC Transcription Factor From Pepper |
| | | | (Capsicum annuum L.).” Front Plant Sci 11: |
| | | | 14. |

Table E lists the representative functional genes in soybean. Methods in the present invention can be used to edit such genes and create new genes by designing new combinations of different gene elements or different protein domain, and can be utilized in soybean breeding program.

TABLE E
Important functional genes in soybean

| Gene name | Gene number | Application | Reference |
|---|---|---|---|
| GmDIR27 | Glyma.05g213400 | Resistance to | Ma, X., et al. (2021). “Functional |
| | | pod cracking | characterization of soybean (Glycine max) |
| | | | DIRIGENT genes reveals an important role of |
| | | | GmDIR27 in the regulation of pod |
| | | | dehiscence.” Genomics 113(1 Pt 2): 979-990. |
| GmG6PDH2 | Glyma.19G082300 | Salt tolerance | Zhao, Y., et al. (2020). “Genome-Wide |
| | | | Analysis of the Glucose-6-Phosphate |
| | | | Dehydrogenase Family in Soybean and |
| | | | Functional Identification of GmG6PDH2 |
| | | | Involvement in Salt Stress.” Front Plant Sci |
| | | | 11: 214. |
| GmPLDalpha1 | Glyma.01G215100 | Root nodule | Zhang, G., et al. (2020). “Phospholipase D- |
| | | development | and phosphatidic acid-mediated phospholipid |
| | | | metabolism and signaling modulate symbiotic |
| | | | interaction and nodulation in soybean (Glycine |
| | | | max).” Plant J. |
| GmGPA3 | Glyma.20G32900 | Growth | Wei, Z., et al. (2020). “GmGPA3 is involved in |
| | | development | post-Golgi trafficking of storage proteins and |
| | | | cell growth in soybean cotyledons.” Plant Sci |
| | | | 294: 110423. |
| GmNMHC5 | Glyma.13G255200 | Development | Wang, W., et al. (2020). “GmNMHC5, A |
| | | stage | Neoteric Positive Transcription Factor of |
| | | | Flowering and Maturity in Soybean.” Plants |
| | | | (Basel) 9(6). |
| GmPRR37 | Glyma.12G073900 | Development | Wang, L., et al. (2020). “Natural variation and |
| | | stage | CRISPR/Cas9-mediated mutation in |
| | | | GmPRR37 affect photoperiodic flowering and |
| | | | contribute to regional adaptation of soybean.” |
| | | | Plant Biotechnol J 18(9): 1869-1881. |
| GmDRR1 | Glyma.11G150400.1 | Root nodule | Shi, Y., et al. (2020). “RNA |
| | | development | Sequencing-Associated Study Identifies |
| | | | GmDRR1 as Positively Regulating the |
| | | | Establishment of Symbiosis in Soybean.” Mol |
| | | | Plant Microbe Interact 33(6): 798-807. |
| GmMYB29A2 | Glyma.02G005600 | Resistance to | Jahan, M. A., et al. (2020). “Glyceollin |
| | | phytophthora | Transcription Factor GmMYB29A2 Regulates |
| | | sojae | Soybean Resistance to Phytophthora sojae.” |
| | | | Plant Physiol 183(2): 530-546. |
| GmMYB68 | Glyma.04G042300.1 | Salt alkali | He, Y., et al. (2020). “Functional activation of |
| | | tolerance | a novel R2R3-MYB protein gene, GmMYB68, |
| | | | confers salt-alkali resistance in soybean |
| | | | (Glycine max L.).” Genome 63(1): 13-26. |
| GmCDF1 | Glyma.08G102000 | Salt tolerance | Zhang, W., et al. (2019). “A cation diffusion |
| | | | facilitator, GmCDF1, negatively regulates salt |
| | | | tolerance in soybean.” PLoS Genet 15(1): |
| | | | e1007798. |
| GmBTB/POZ | Glyma.04G244900 | Resistance to | Zhang, C., et al. (2019). “GmBTB/POZ, a |
| | | phytophthora | novel BTB/POZ domain-containing nuclear |
| | | sojae | protein, positively regulates the response of |
| | | | soybean to Phytophthora sojae infection.” Mol |
| | | | Plant Pathol 20(1): 78-91. |
| GmSnRK1.1 | Glyma.08G240300 | Resistance to | Wang, L., et al. (2019). “GmSnRK1.1, a |
| | | phytophthora | Sucrose Non-fermenting-1(SNF1)-Related |
| | | sojae | Protein Kinase, Promotes Soybean Resistance |
| | | | to Phytophthora sojae.” Front Plant Sci 10: |
| | | | 996. |
| GmSIN1 | Glyma.12G221500.1 | Salt tolerance | Li, S., et al. (2019). “A |
| | | | GmSIN1/GmNCED3s/GmRbohBs |
| | | | Feed-Forward Loop Acts as a Signal Amplifier |
| | | | That Regulates Root Growth in Soybean |
| | | | Exposed to Salt Stress.” Plant Cell 31(9): |
| | | | 2107-2130. |
| GmHsp90A2 | Glyma.16G178800 | Thermo | Huang, Y., et al. (2019). “GmHsp90A2 is |
| | | tolerance | involved in soybean heat stress as a positive |
| | | | regulator.” Plant Sci 285: 26-33. |
| GmPI4L | NM.001256363.1 | Resistance to | Chen, X., et al. (2019). “Overexpression of a |
| | | phytophthora | soybean 4-coumaric acid: coenzyme A ligase |
| | | sojae | (GmPI4L) enhances resistance to Phytophthora |
| | | | sojae in soybean.” Funct Plant Biol 46(4): |
| | | | 304-313. |
| GmPT7 | Glyma.14G188000 | Root nodule | Chen, L., et al. (2019). “A nodule-localized |
| | | development; | phosphate transporter GmPT7 plays an |
| | | increase yield | important role in enhancing symbiotic N2 |
| | | | fixation and yield in soybean.” New Phytol |
| | | | 221(4): 2013-2025. |
| GmbZIP1 | Glyma.02G131700 | Root nodule | Xu, S., et al. (2021). “GmbZIP1 negatively |
| | | development | regulates ABA-induced inhibition of |
| | | | nodulation by targeting GmENOD40-1 in |
| | | | soybean.” BMC Plant Biol 21(1): 35. |
| GmCRY1b | Glyma.06G103200 | Tolerance to | Lyu, X., et al. (2021). “GmCRY1s modulate |
| | | close planting | gibberellin metabolism to regulate soybean |
| | | | shade avoidance in response to reduced blue |
| | | | light.” Mol Plant 14(2): 298-314. |
| GmNAC06 | Glyma.06g21020.1 | Salt tolerance | Li, M., et al. (2021). “GmNAC06, aNAC |
| | | | domain transcription factor enhances salt stress |
| | | | tolerance in soybean.” Plant Mol Biol 105(3): |
| | | | 333-345. |
| GmbZIP2 | Glyma.14G002300 | Salt and | Yang, Y., et al. (2020). “The Soybean bZIP |
| | | drought | Transcription Factor Gene GmbZIP2 Confers |
| | | tolerance | Drought and Salt Resistances in Transgenic |
| | | | Plants.” Int J Mol Sci 21(2). |
| GmNAC8 | Glyma.16G151500.1 | Drought | Yang, C., et al. (2020). “GmNAC8 acts as a |
| | | tolerance | positive regulator in soybean drought stress.” |
| | | | Plant Sci 293: 110442. |
| GmPAP12 | Glyma.06G028200 | Root nodule | Wang, Y., et al. (2020). “GmPAP12 Is |
| | | development | Required for Nodule Development and |
| | | | Nitrogen Fixation Under Phosphorus |
| | | | Starvation in Soybean.” Front Plant Sci 11: |
| | | | 450. |
| GmNFYA13 | Glyma.13G202300 | Salt and | Ma, X. J., et al. (2020). “GmNFYA13 |
| | | drought | Improves Salt and Drought Tolerance in |
| | | tolerance | Transgenic Soybean Plants.” Front Plant Sci |
| | | | 11: 587244. |
| GmAAP6a | Glyma.17g192000 | Tolerance to | Liu, S., et al. (2020). “Overexpression of |
| | | nitrogen | GmAAP6a enhances tolerance to low nitrogen |
| | | deficiency | and improves seed nitrogen status by |
| | | | optimizing amino acid partitioning in |
| | | | soybean.” Plant Biotechnol J 18(8): |
| | | | 1749-1762. |
| GmPRR3b | Glyma.12G073900.1 | To regulate | Li, C., et al. (2020). “A |
| | | development | Domestication-Associated Gene GmPRR3b |
| | | stage | Regulates the Circadian Clock and Flowering |
| | | | Time in Soybean.” Mol Plant 13(5): 745-759. |
| GmMYB14 | Glyma.15G259400 | Tolerance to | Chen, L., et al. (2020). “Overexpression of |
| | | close planting | GmMYBl4 improves high-density yield and |
| | | and drought | drought tolerance of soybean through |
| | | | regulating plant architecture mediated by the |
| | | | brassinosteroid pathway.” Plant Biotechnol J. |
| GmAP1 | Glyma.16G091300 | To increase | Chen, L., et al. (2020). “Soybean AP1 |
| | | yield | homologs control flowering time and plant |
| | | | height.” J Integr Plant Biol 62(12): 1868-1879. |
| GmUBC9 | Glyma.03G199900 | Drought | Chen, K., et al. (2020). “Overexpression of |
| | | tolerance; late | GmUBC9 Gene Enhances Plant Drought |
| | | maturing | Resistance and Affects Flowering Time via |
| | | | Histone H2B Monoubiquitination.” Front Plant |
| | | | Sci 11: 555794. |
| GmOLEO1 | Glyma.20G196600 | High seed oil | Zhang, D., et al. (2019). “Artificial selection |
| | | content | on GmOLEO1 contributes to the increase in |
| | | | seed oil during soybean domestication.” PLoS |
| | | | Genet 15(7): e1008267. |
| GmKR3 | Glyma.06G267300 | Resistance to | Xun, H., et al. (2019). “Over-expression of |
| | | viral diseases | GmKR3, a TIR-NBS-LRR type R gene, |
| | | | confers resistance to multiple viruses in |
| | | | soybean.” Plant Mol Biol 99(1-2): 95-111. |
| GmYUC2a | Glyma.08G038600 | Root nodule | Wang, Y., et al. (2019). “GmYUC2a mediates |
| | | development | auxin biosynthesis during root development |
| | | | and nodulation in soybean.” J Exp Bot 70(12): |
| | | | 3165-3176. |
| GmNFR1alpha | Glyma.02G270800 | Root nodule | Indrasumunar, A., et al. (2011). “Nodulation |
| | | development | factor receptor kinase 1alpha controls nodule |
| | | | organ number in soybean (Glycine max L. |
| | | | Merr).” Plant J 65(1): 39-50. |
| GmHsfA1 | Glyma.16G091800.1 | Thermo | Zhu, B., et al. (2006). “Identification and |
| | | tolerance | characterization of a novel heat shock |
| | | | transcription factor gene, GmHsfA1, in |
| | | | soybeans (Glycine max).” J Plant Res 119(3): |
| | | | 247-256. |
| GmMPK1 | Glyma.08G309500 | To enhance | Wu, D., et al. (2020). “Identification of a |
| | | resistance to | candidate gene associated with isoflavone |
| | | phytophthora | content in soybean seeds using genome-wide |
| | | sojae; to | association and linkage mapping.” Plant J |
| | | increase | 104(4): 950-963. |
| | | isoflavone | |
| | | content | |
| GmIFR | NM_001254100 | Resistance to | Cheng, Q., et al. (2015). “Overexpression of |
| | | phytophthora | Soybean Isoflavone Reductase (GmIFR) |
| | | sojae in | Enhances Resistance to Phytophthora sojae in |
| | | soybean | Soybean.” Front Plant Sci 6: 1024. |
| GmCnx1 | NM_001255600 | Resistance to | Zhou, Z., et al. (2015). “Overexpression of a |
| | | mosaic virus | GmCnxl gene enhanced activity of nitrate |
| | | SMV | reductase and aldehyde oxidase, and boosted |
| | | | mosaic virus resistance in soybean.” PLoS One |
| | | | 10(4): e0124273. |
| GmPRP | KM506762 | Resistance to | Jiang, L., et al. (2015). “Isolation and |
| | | phytophthora | Characterization of a Novel |
| | | sojae No. 1 | Pathogenesis-Related Protein Gene (GmPRP) |
| | | physiological | with Induced Expression in Soybean (Glycine |
| | | race in | max) during Infection with Phytophthora |
| | | soybean | sojae.” PLoS One 10(6): e0129932. |
| GmIFR | NM_001254100, | Resistance to | Cheng, Q., et al. (2015). “Overexpression of |
| | | phytophthora | Soybean Isoflavone Reductase (GmIFR) |
| | | sojae in | Enhances Resistance to Phytophthora sojae in |
| | | soybean | Soybean.” Front Plant Sci 6: 1024. |
| GmCBS21 | Glyma.06G032200 | Nitrogen use | Hao, Q., et al. (2016). “Identification and |
| | | efficiency | Comparative Analysis of CBS |
| | | | Domain-Containing Proteins in Soybean |
| | | | (Glycine max) and the Primary Function of |
| | | | GmCBS21 in Enhanced Tolerance to Low |
| | | | Nitrogen Stress.” Int J Mol Sci 17(5). |
| GA20OX, | Glyma07g08950, | Seed weight | Lu, X., et al. (2016). “The transcriptomic |
| NFYA | Glyma02g47380 | and seed oil | signature of developing soybean seeds reveals |
| | | content | the genetic basis of seed trait adaptation during |
| | | | domestication.” Plant J 86(6): 530-544. |
| GmDIR22 | HQ_993047 | Resistance to | Li, N., et al. (2017). “A Novel Soybean |
| | | phytophthora | Dirigent Gene GmDIR22 Contributes to |
| | | sojae in | Promotion of Lignan Biosynthesis and |
| | | soybean | Enhances Resistance to Phytophthora sojae.” |
| | | | Front Plant Sci 8: 1185. |
| GmZF351 | Glyma06g44440 | To increase | Li, Q. T., et al. (2017). “Selection for a |
| | | seed oil | Zinc-Finger Protein Contributes to Seed Oil |
| | | content in | Increase during Soybean Domestication.” Plant |
| | | soybean | Physiol 173(4): 2208-2224. |
| GmORG3 | Glyma03g28630 | Resistance to | Xu, Z., et al. (2017). “The Soybean Basic |
| | | chromium | Helix-Loop-Helix Transcription Factor |
| | | stress | ORG3-Like Enhances Cadmium Tolerance via |
| | | | Increased Iron and Reduced Cadmium Uptake |
| | | | and Transport from Roots to Shoots.” Front |
| | | | Plant Sci 8: 1098. |
| GmESR1 | JN590243.1 | To promote | Zhang, C., et al. (2017). “Functional analysis |
| | | seed | of the GmESR1 gene associated with soybean |
| | | germination | regeneration.” PLoS One 12(4): e0175656. |
| GmAGL1 | AW433203 | To promote | Zeng, X., et al. (2018). “Soybean MADS-box |
| | | plant | gene GmAGL1 promotes flowering via the |
| | | maturation for | photoperiod pathway.” BMC Genomics 19(1): |
| | | early | 51. |
| | | flowering and | |
| | | early maturing | |
| GmPIP1; 6 | Gm08g01860.1 | Salt tolerance | Zhou, L., et al. (2014). “Constitutive |
| | | | overexpression of soybean plasma membrane |
| | | | intrinsic protein GmPIP1; 6 confers salt |
| | | | tolerance.” BMC Plant Biol 14: 181. |
| GmAKT2 | Glym08g20030.1 | SMV tolerance | Zhou, L., et al. (2014). “Overexpression of |
| | | | GmAKT2 potassium channel enhances |
| | | | resistance to soybean mosaic virus.” BMC |
| | | | Plant Biol 14: 154. |

Table F lists the representative functional genes in corn. Methods in the present invention can be used to edit such genes and create new genes by designing new combinations of different gene elements or different protein domain, and can be utilized in corn breeding program.

TABLE F
Important functional genes in corn

| Gene name | Application | Reference |
|---|---|---|
| ZmMKK1 | Drought and salt | Cai, G., et al. (2014). “A maize mitogen-activated protein |
| | tolerance | kinase kinase, ZmMKK1, positively regulated the salt and |
| | | drought tolerance in transgenic Arabidopsis.” J Plant Physiol |
| | | 171(12): 1003-1016. |
| ZmCesA7 | To increase | de Castro, M., et al. (2014). “Early cell-wall modifications of |
| | cellulose content in | maize cell cultures during habituation to dichlobenil.” J Plant |
| | cells | Physiol 171(2): 127-135. |
| ZmCesA8 | To increase | de Castro, M., et al. (2014). “Early cell-wall modifications of |
| | cellulose content in | maize cell cultures during habituation to dichlobenil.” J Plant |
| | cells | Physiol 171(2): 127-135. |
| ZmARGOS1 | To increase grain | Guo, M., et al. (2014). “Maize ARGOS1 (ZAR1) transgenic |
| | yield and improve | alleles increase hybrid maize yield.” J Exp Bot 65(1): |
| | drought tolerance | 249-260. |
| ZmARF25 | To control corn leaf | Li, C., et al. (2014). “Ectopic expression of a maize hybrid |
| | size | down-regulated gene ZmARF25 decreases organ size by |
| | | affecting cellular proliferation in Arabidopsis.” PLoS One |
| | | 9(4): e94830. |
| ZmLEA5C | Stress resistance | Liu, Y., et al. (2014). “Group 5 LEA protein, ZmLEA5C, |
| | | enhance tolerance to osmotic and low temperature stresses in |
| | | transgenic tobacco and yeast.” Plant Physiol Biochem 84: |
| | | 22-31. |
| ZmVP1 | Seed development | Suzuki, M., et al. (2014). “Distinct functions of COAR and |
| | | B3 domains of maize VP1 in induction of ectopic gene |
| | | expression and plant developmental phenotypes in |
| | | Arabidopsis.” Plant Mol Biol 85(1-2): 179-191. |
| ZmRACK1 | Disease resistance | Wang, B., et al. (2014). “Maize ZmRACK1 is involved in the |
| | | plant response to fungal phytopathogens.” Int J Mol Sci |
| | | 15(6): 9343-9359. |
| ZmGRF10 | To affect leaf size | Wu, L., et al. (2014). “Overexpression of the maize GRF10, |
| | and plant height | an endogenous truncated growth-regulating factor protein, |
| | | leads to reduction in leaf size and plant height.” J Integr Plant |
| | | Biol 56(11): 1053-1063. |
| Zm | To increase urea | Zanin, L., et al. (2014). “Isolation and functional |
| urea-proton | uptake | characterization of a high affinity urea transporter from roots |
| symporter | | of Zea mays.” BMC Plant Biol 14: 222. |
| DUR3 | | |
| ZmMPK5 | To participate in | Zhang, D., et al. (2014). “The overexpression of a maize |
| | signal transduction | mitogen-activated protein kinase gene (ZmMPK5) confers |
| | pathway of salt | salt stress tolerance and induces defence responses in |
| | stress, oxidative | tobacco.” Plant Biol (Stuttg) 16(3): 558-570. |
| | stress and pathogen | |
| | defense | |
| ZmSOC1 | Early flowering | Zhao, S., et al. (2014). “ZmSOC1, a MADS-box transcription |
| | | factor from Zea mays, promotes flowering in Arabidopsis.” |
| | | Int J Mol Sci 15(11): 19987-20003. |
| Zmhdz10 | Drought and salt | Zhao, Y., et al. (2014). “A novel maize homeodomain-leucine |
| | tolerance | zipper (HD-Zip) I gene, Zmhdz10, positively regulates |
| | | drought and salt tolerance in both rice and Arabidopsis.” |
| | | Plant Cell Physiol 55(6): 1142-1156. |
| ZmPIF3 | Drought and salt | Gao, Y., et al. (2015). “A maize phytochrome-interacting |
| | tolerance | factor 3 improves drought and salt stress tolerance in rice.” |
| | | Plant Mol Biol 87(4-5): 413-428. |
| ZmpsbA | Drought tolerance | Huo, Y., et al. (2015). “Overexpression of the Maize psbA |
| | | Gene Enhances Drought Tolerance Through Regulating |
| | | Antioxidant System, Photosynthetic Capability, and Stress |
| | | Defense Gene Expression in Tobacco.” Front Plant Sci 6: |
| | | 1223. |
| ZmIRT1 | Iron uptake | Li, S., et al. (2015). “Overexpression of ZmIRT1 and |
| | | ZmZIP3 Enhances Iron and Zinc Accumulation in Transgenic |
| | | Arabidopsis.” PLoS One 10(8): e0136647. |
| ZmZIP3 | Zinc uptake | Li, S., et al. (2015). “Overexpression of ZmIRT1 and |
| | | ZmZIP3 Enhances Iron and Zinc Accumulation in Transgenic |
| | | Arabidopsis.” PLoS One 10(8): e0136647. |
| ZmBDF | Drought and salt | Liu, Y., et al. (2015). “Characterization and functional |
| | tolerance | analysis of a B3 domain factor from Zea mays.” J Appl Genet |
| | | 56(4): 427-438. |
| ZmARGOS8 | Drought tolerance, | Shi, J., et al. (2015). “Overexpression of ARGOS Genes |
| | yield increase | Modifies Plant Sensitivity to Ethylene, Leading to Improved |
| | | Drought Tolerance in Both Arabidopsis and Maize.” Plant |
| | | Physiol 169(1): 266-282. |
| ZmCPK1 | Cold stress | Weckwerth, P., et al. (2015). “ZmCPK1, a |
| | | calcium-independent kinase member of the Zea mays CDPK |
| | | gene family, functions as a negative regulator in cold stress |
| | | signalling.” Plant Cell Environ 38(3): 544-558. |
| ZmMAPK1 | Drought tolerance | Wu, L., et al. (2015). “Overexpression of ZmMAPK1 |
| | and thermos stress | enhances drought and heat stress in transgenic Arabidopsis |
| | | thaliana.” Plant Mol Biol 88(4-5): 429-443. |
| ZmGRF | To promote plant | Xu, M., et al. (2015). “ZmGRF, a GA regulatory factor from |
| | flowering, stem | maize, promotes flowering and plant growth in Arabidopsis.” |
| | elongation and cell | Plant Mol Biol 87(1-2): 157-167. |
| | expansion, GA | |
| | singal | |
| ZmCCaMK | Antioxidant defense | Yan, J., et al. (2015). “Calcium and ZmCCaMK are involved |
| | | in brassinosteroid-induced antioxidant defense in maize |
| | | leaves.” Plant Cell Physiol 56(5): 883-896. |
| ZmJAZ14 | Drought tolerance | Zhou, X., et al. (2015). “A maize jasmonate Zim-domain |
| | and growth | protein, ZmJAZ14, associates with the JA, ABA, and GA |
| | promotion | signaling pathways in transgenic Arabidopsis.” PLoS One |
| | regulation | 10(3): e0121824. |
| ZmMADS1 | Early flowering | Alter, P., et al. (2016). “Flowering Time-Regulated Genes in |
| | | Maize Include the Transcription Factor ZmMADS1.” Plant |
| | | Physiol 172(1): 389-404. |
| ZmSAD1 | To adjust contents | Du, H., et al. (2016). “Modification of the fatty acid |
| | of stearic acid, oil | composition in Arabidopsis and maize seeds using a |
| | acid and long chain | stearoyl-acyl carrier protein desaturase-1 (ZmSAD1) gene.” |
| | saturated acid and | BMC Plant Biol 16(1): 137. |
| | the proportion of | |
| | saturated fatty acid | |
| | and unsaturated | |
| | fatty acid | |
| ZmGOLS2 | Stress resistance | Gu, L., et al. (2016). “ZmGOLS2, a target of transcription |
| | | factor ZmDREB2A, offers similar protection against abiotic |
| | | stress as ZmDREB2A.” Plant Mol Biol 90(1-2): 157-170. |
| ZmOXS2b | Stress resistance | He, L., et al. (2016). “Maize OXIDATIVE STRESS2 |
| | | Homologs Enhance Cadmium Tolerance in Arabidopsis |
| | | through Activation of a Putative SAM-Dependent |
| | | Methyltransferase Gene.” Plant Physiol 171(3): 1675-1685. |
| ZmO2L1 | Stress resistance | He, L., et al. (2016). “Maize OXIDATIVE STRESS2 |
| | | Homologs Enhance Cadmium Tolerance in Arabidopsis |
| | | through Activation of a Putative SAM-Dependent |
| | | Methyltransferase Gene.” Plant Physiol 171(3): 1675-1685. |
| ZmEREB156 | Starch synthesis | Huang, H., et al. (2016). “Sucrose and ABA regulate starch |
| | | biosynthesis in maize through a novel transcription factor, |
| | | ZmEREB156.” Sci Rep 6: 27590. |
| ZmZIP7 | To stimulate | Li, S., et al. (2016). “Constitutive expression of the ZmZIP7 |
| | endogenous iron and | in Arabidopsis alters metal homeostasis and increases Fe and |
| | zinc uptake | Zn content.” Plant Physiol Biochem 106: 1-10. |
| ZmLEA3 | To enhance | Liu, Y., et al. (2016). “Group 3 LEA Protein, ZmLEA3, Is |
| | tolerance to cold | Involved in Protection from Low Temperature Stress.” Front |
| | stress | Plant Sci 7: 1011. |
| Baby boom | To improve | Lowe, K., et al. (2016). “Morphogenic Regulators Baby |
| (BBM) | transformation | boom and Wuschel Improve Monocot Transformation.” Plant |
| | efficiency | Cell 28(9): 1998-2015. |
| Wuschel2 | To improve | Lowe, K., et al. (2016). “Morphogenic Regulators Baby |
| | transformation | boom and Wuschel Improve Monocot Transformation.” Plant |
| | efficiency | Cell 28(9): 1998-2015. |
| ZmABA2 | Drought and salt | Ma, F., et al. (2016). “ZmABA2, an interacting protein of |
| | tolerance | ZmMPK5, is involved in abscisic acid biosynthesis and |
| | | functions.” Plant Biotechnol J 14(2): 771-782. |
| ZmNAC55 | Drought tolerance | Mao, H., et al. (2016). “ZmNAC55, a maize stress-responsive |
| | | NAC transcription factor, confers drought resistance in |
| | | transgenic Arabidopsis.” Plant Physiol Biochem 105: 55-66. |
| ZmVPP5 | To enhance salt | Sun, X., et al. (2016). “Maize ZmVPP5 is a truncated |
| | sensitivity | Vacuole H(+) -PPase that confers hypersensitivity to salt |
| | | stress.” J Integr Plant Biol 58(6): 518-528. |
| ZmArf2 | Active GTP | Wang, Q., et al. (2016). “A maize ADP-ribosylation factor |
| | combination, | ZmArf2 increases organ and seed size by promoting cell |
| | endosperm | expansion in Arabidopsis.” Physiol Plant 156(1): 97-107. |
| | development | |
| ZmSEC14p | Cold stress | Wang, X., et al. (2016). “Isolation and functional |
| | resistance | characterization of a cold responsive phosphatidylinositol |
| | | transfer-associated protein, ZmSEC14p, from maize (Zea |
| | | may L.).” Plant Cell Rep 35(8): 1671-1686. |
| ZmCBL9 | Stress resistance | Zhang, F., et al. (2016). “Characterization of the calcineurin |
| | | B-Like (CBL) gene family in maize and functional analysis |
| | | of ZmCBL9 under abscisic acid and abiotic stress |
| | | treatments.” Plant Sci 253: 118-129. |
| ZmXerico1 | Drought tolerance | Brugiere, N., et al. (2017). “Overexpression of RING Domain |
| | | E3 Ligase ZmXerico1 Confers Drought Tolerance through |
| | | Regulation of ABA Homeostasis.” Plant Physiol 175(3): |
| | | 1350-1369. |
| ZmXerico2 | Drought tolerance | Brugiere, N., et al. (2017). “Overexpression of RING Domain |
| | | E3 Ligase ZmXerico1 Confers Drought Tolerance through |
| | | Regulation of ABA Homeostasis.” Plant Physiol 175(3): |
| | | 1350-1369. |
| ZmGRAS20 | Starch synthesis | Cai, H., et al. (2017). “A novel GRAS transcription factor, |
| | | ZmGRAS20, regulates starch biosynthesis in rice |
| | | endosperm.” Physiol Mol Biol Plants 23(1): 143-154. |
| ZmWRKY17 | Salt stress response | Cai, R., et al. (2017). “The maize WRKY transcription factor |
| | | ZmWRKY17 negatively regulates salt stress tolerance in |
| | | transgenic Arabidopsis plants.” Planta 246(6): 1215-1231. |
| ZmNLP6 | Nitrogen utilization | Cao, H., et al. (2017). “Overexpression of the Maize |
| | | ZmNLP6 and ZmNLP8 Can Complement the Arabidopsis |
| | | Nitrate Regulatory Mutant nlp7 by Restoring Nitrate |
| | | Signaling and Assimilation.” Front Plant Sci 8: 1703. |
| ZmNLP8 | Nitrogen utilization | Cao, H., et al. (2017). “Overexpression of the Maize |
| | | ZmNLP6 and ZmNLP8 Can Complement the Arabidopsis |
| | | Nitrate Regulatory Mutant nlp7 by Restoring Nitrate |
| | | Signaling and Assimilation.” Front Plant Sci 8: 1703. |
| TRU1 | To improve plant | Dong, Z., et al. (2017). “Ideal crop plant architecture is |
| | morphology | mediated by tassels replace upper ears1, a BTB/POZ ankyrin |
| | | repeat gene directly targeted by TEOSINTE BRANCHED1.” |
| | | Proc Natl Acad Sci USA 114(41): E8656-E8664. |
| UNBRANCH | To regulate | Du, Y., et al. (2017). “UNBRANCHED3 regulates branching |
| ED3/UB3 | vegetative and | by modulating cytokinin biosynthesis and signaling in maize |
| | reproductive | and rice.” New Phytol 214(2): 721-733. |
| | branching | |
| ZmWRKY4 | Oxidation resistance | Hong, C., et al. (2017). “The role of ZmWRKY4 in |
| | | regulating maize antioxidant defense under cadmium stress.” |
| | | Biochem Biophys Res Commun 482(4): 1504-1510. |
| ZmMGT10 | To enhance | Li, H., et al. (2017). “The maize CorA/MRS2/MGT-type Mg |
| | tolerance to Mg | transporter, ZmMGT10, responses to magnesium deficiency |
| | deficiency in corn | and confers low magnesium tolerance in transgenic |
| | | Arabidopsis.” Plant Mol Biol 95(3): 269-278 |
| ZmGOLS2 | To increase seed | Li, T., et al. (2017). “Regulation of Seed Vigor by |
| | vigor | Manipulation of Raffinose Family Oligosaccharides in Maize |
| | | and Arabidopsis thaliana.” Mol Plant 10(12): 1540-1555. |
| ZmRS | To reduce seed | Li, T., et al. (2017). “Regulation of Seed Vigor by |
| | vigor | Manipulation of Raffinose Family Oligosaccharides in Maize |
| | | and Arabidopsis thaliana.” Mol Plant 10(12): 1540-1555. |
| ZmINCW1 | To increase grain | Liu, J., et al. (2017). “The Conserved and Unique Genetic |
| | size/weight | Architecture of Kernel Size and Weight in Maize and Rice.” |
| | | Plant Physiol 175(2): 774-785. |
| ZmDHN13 | To enhance | Liu, Y., et al. (2017). “Functional characterization of KS-type |
| | tolerance to | dehydrin ZmDHN13 and its related conserved domains under |
| | oxidative stress | oxidative stress.” Sci Rep 7(1): 7361. |
| ZmPIF4 | To respond to | Shi, Q., et al. (2017). “Functional Characterization of the |
| | phytochrome singals | Maize Phytochrome-Interacting Factors PIF4 and PIF5.” |
| | | Front Plant Sci 8: 2273. |
| ZmPIF5 | To respond to | Shi, Q., et al. (2017). “Functional Characterization of the |
| | phytochrome singals | Maize Phytochrome-Interacting Factors PIF4 and PIF5.” |
| | | Front Plant Sci 8: 2273. |
| ABP9 | Stress resistance | Wang, C., et al. (2017). “ABP9, a maize bZIP transcription |
| | | factor, enhances tolerance to salt and drought in transgenic |
| | | cotton.” Planta 246(3): 453-469. |
| ZmPP2AA1 | Low phosphate | Wang, J., et al. (2017). “Overexpression of the protein |
| | response | phosphatase 2A regulatory subunit a gene ZmPP2AA1 |
| | | improves low phosphate tolerance by remodeling the root |
| | | system architecture of maize.” PLoS One 12(4): e0176538. |
| ZmMYB14 | Starch synthesis | Xiao, Q., et al. (2017). “ZmMYB14 is an important |
| | | transcription factor involved in the regulation of the activity |
| | | of the ZmBT1 promoter in starch biosynthesis in maize.” |
| | | FEBS J 284(18): 3079-3099. |
| ZmPR10 | Resistance to plant | Zandvakili, N., et al. (2017). “Cloning, Overexpression and |
| | pathogenic fungi | in vitro Antifungal Activity of Zea Mays PR10 Protein.” Iran |
| | | J Biotechnol 15(1): 42-49. |
| SFA1 | Cellulose hydrolysis | Zhu, L., et al. (2020). Overexpression of SFA1 in engineered |
| | | Saccharomyces cerevisiae to increase xylose utilization and |
| | | ethanol production from different lignocellulose |
| | | hydrolysates. Bioresour Technol 313, 123724. |
| ZmSCE1b | Paraquat resistance | Wang, H., et al. (2021). The maize SUMO conjugating |
| | | enzyme ZmSCE1b protects plants from paraquat toxicity. |
| | | Ecotoxicol Environ Saf 211, 111909. |
| ZmEREB20 | Salt stress resistance | Fu, J., et al. (2021). Maize transcription factor ZmEREB20 |
| | | enhanced salt tolerance in transgenic Arabidopsis. Plant |
| | | Physiol Biochem 159, 257-267. |
| ZmMPKL1 | Drought tolerance | Zhu, D., et al. (2020). MAPK-like protein 1 positively |
| | | regulates maize seedling drought sensitivity by suppressing |
| | | ABA biosynthesis. Plant J 102, 747-760. |
| ZmCCD10a | Phosphate stress | Zhong, Y., et al. (2020). ZmCCD10a Encodes a Distinct Type |
| | | of Carotenoid Cleavage Dioxygenase and Enhances Plant |
| | | Tolerance to Low Phosphate. Plant Physiol 184, 374-392. |
| ZmBZR1 | Organ development | Zhang, X., Guo, W., Du, D., Pu, L., and Zhang, C. (2020). |
| | | Overexpression of a maize BR transcription factor ZmBZR1 |
| | | in Arabidopsis enlarges organ and seed size of the transgenic |
| | | plants. Plant Sci 292, 110378. |
| ZmTMT | Quality | Zhang, L., et al. (2020). Overexpression of the maize |
| | improvement | gamma-tocopherol methyltransferase gene (ZmTMT) |
| | | increases alpha-tocopherol content in transgenic Arabidopsis |
| | | and maize seeds. Transgenic Res 29, 95-104. |
| ZmPTPN | Drought tolerance | Zhang, H., et al. (2020). Enhanced Vitamin C Production |
| | | Mediated by an ABA-Induced PTP-like Nucleotidase |
| | | Improves Plant Drought Tolerance in Arabidopsis and Maize. |
| | | Mol Plant 13, 760-776. |
| ZmMYB59 | Seed germination | Zhai, K., et al. (2020). Overexpression of Maize ZmMYB59 |
| | | Gene Plays a Negative Regulatory Role in Seed Germination |
| | | in Nicotiana tabacum and Oryza sativa. Front Plant Sci 11, |
| | | 564665. |
| ZmERF105 | Resistance to | Zang, Z., et al. (2020). A Novel ERF Transcription Factor, |
| | exserohilum | ZmERF105, Positively Regulates Maize Resistance to |
| | turcicum | Exserohilum turcicum. Front Plant Sci 11, 850. |
| ZmNAC126 | To accelerate | Yang, Z., et al. (2020). The transcription factor ZmNAC126 |
| | maturation | accelerates leaf senescence downstream of the ethylene |
| | | signalling pathway in maize. Plant Cell Environ 43, |
| | | 2287-2300. |
| EMP32 | Seed development | Yang, Y. Z., et al. (2020). EMP32 is required for the |
| | | cis-splicing of nad7 intron 2 and seed development in maize. |
| | | RNA Biol, 1-11. |
| ZmPt9 | Phosphate | Xu, Y., et al. (2020). Overexpression of a phosphate |
| | transportation to | transporter gene ZmPt9 from maize influences growth of |
| | promote crop | transgenic Arabidopsis thaliana. Biochem Biophys Res |
| | growth | Commun. |
| ZmNAC49 | Drought tolerance | Xiang, Y., et al. (2020). ZmNAC49 reduces stomatai density |
| | | to improve drought tolerance in maize. J Exp Bot. |
| NIGT1.2 | To maintain | Wang, X., et al. (2020). The Transcription Factor NIGT1.2 |
| | nitrogen and | Modulates Both Phosphate Uptake and Nitrate Influx during |
| | phosphorus balance | Phosphate Starvation in Arabidopsis and Maize. Plant Cell |
| | | 32, 3519-3534. |
| ZmmCCHA1 | Photosynthesis | Wang, C., et al.(2020). Functional characterization of a |
| | | chloroplast-localized Mn(2+)(Ca(2+))/H(+) antiporter, |
| | | ZmmCCHA1 from Zea mays ssp. mexicana L. Plant Physiol |
| | | Biochem 155, 396-405. |
| ZmBES1/BZR1-5 | Grain development, | Sun, F., et al. (2020). Maize transcription factor |
| | yield increase | ZmBES1/BZR1-5 positively regulates kernel size. J Exp Bot. |
| ZM-BG1H1 | Yield increase | Simmons, C. R., et al. (2020). Maize BIG GRAIN1 homolog |
| | | overexpression increases maize grain yield. Plant Biotechnol |
| | | J 18, 2304-2315. |
| ZmCCA1a | Photoperiod | Shi, Y., et al. (2020). ZmCCA1a on Chromosome 10 of |
| | regulation | Maize Delays Flowering of Arabidopsis thaliana. Front Plant |
| | | Sci 11, 78. |
| Dtbn1 | To control tassel | Qin, X., et al. (2020). Q(Dtbn1), an F-box gene affecting |
| | branch number | maize tassel branch number by a dominant model. Plant |
| | | Biotechnol J. |
| ZmTMM1 | To regulate lateral | Liu, Y., et al. (2020). Involvement of a truncated MADS-box |
| | root development | transcription factor ZmTMM1 in root nitrate foraging. J Exp |
| | | Bot 71, 4547-4561. |
| Zm-miR164e | To increase branch | Liu, M., et al. (2020). Analysis of the genetic architecture of |
| | number | maize kernel size traits by combined linkage and association |
| | | mapping. Plant Biotechnol J 18, 207-221. |
| ZmRAFS | Drought tolerance | Li, T., et al. (2020). Raffinose synthase enhances drought |
| | | tolerance through raffinose synthesis or galactinol hydrolysis |
| | | in maize and Arabidopsis plants. J Biol Chem 295, |
| | | 8064-8077. |
| ZmPHYC1 | To drawf plant | Li, Q., et al. (2020). CRISPR/Cas9-mediated knockout and |
| ZmPHYC2 | height and spike | overexpression studies reveal a role of maize phytochrome C |
| | height | in regulating flowering time and plant height. Plant |
| | | Biotechnol J 18, 2520-2532. |
| GRF5 | To increase | Kong, J., et al. (2020). Overexpression of the Transcription |
| | transformation | Factor GROWTH-REGULATING FACTOR5 Improves |
| | efficiency | Transformation of Dicot and Monocot Species. Front Plant |
| | | Sci 11, 572319. |
| KNR6 | Yield increase | Jia, H., et al. (2020). A serine/threonine protein kinase |
| | | encoding gene KERNEL NUMBER PER ROW6 regulates |
| | | maize grain yield. Nat Commun 11, 988. |
| ZmRLK7 | To regulate plant | He, C., et al. (2020). Overexpression of an Antisense RNA of |
| | structure and organ | Maize Receptor-Like Kinase Gene ZmRLK7 Enlarges the |
| | size | Organ and Seed Size of Transgenic Arabidopsis Plants. Front |
| | | Plant Sci 11, 579120. |
| ZmDREB1A | Cold tolerance | Han, Q., et al. (2020). ZmDREB1A Regulates RAFFINOSE |
| | | SYNTHASE Controlling Raffinose Accumulation and Plant |
| | | Chilling Stress Tolerance in Maize. Plant Cell Physiol 61, |
| | | 331-341. |
| ZmDREB2A | To regulate corn | Han, Q., et al. (2020). ZmDREB2A regulates ZmGH3.2 and |
| | seed longevity and | ZmRAFS, shifting metabolism towards seed aging tolerance |
| | increase aging | over seedling growth. Plant J 104, 268-282. |
| | tolerance | |
| ZmMYC2 | To regulate JA | Fu, J., et al. (2020). ZmMYC2 exhibits diverse functions and |
| | mediated growth, | enhances JA signaling in transgenic Arabidopsis. Plant Cell |
| | development and | Rep 39, 273-288. |
| | defensive reaction | |
| CENH3 | Development | Feng, C., et al. (2020). The deposition of CENH3 in maize is |
| | regulation | stringently regulated. Plant J 102, 6-17. |
| ZmAT6 | To enhance | Du, H., et al. (2020). A Maize ZmAT6 Gene Confers |
| | tolerance to | Aluminum Tolerance via Reactive Oxygen Species |
| | aluminum toxicity | Scavenging. Front Plant Sci 11, 1016. |
| | in corn to scavenge | |
| | active oxygen | |
| | species | |
| PIP2; 5 | Drought tolerance | Ding, L., et al. (2020). Modification of the Expression of the |
| | and yield increase | Aquaporin ZmPIP2; 5 Affects Water Relations and Plant |
| | | Growth. Plant Physiol 182, 2154-2165. |
| ZmVPS29 | To promote grain | Chen, L., et al. (2020). The retromer protein ZmVPS29 |
| | development | regulates maize kernel morphology likely through an |
| | | auxin-dependent process(es). Plant Biotechnol J 18, |
| | | 1004-1014. |
| ZmOSCA | Drought tolerance | Cao, L., et al. (2020). Systematic Analysis of the Maize |
| | | OSCA Genes Revealing ZmOSCA Family Members Involved |
| | | in Osmotic Stress and ZmOSCA2.4 Confers Enhanced |
| | | Drought Tolerance in Transgenic Arabidopsis. Int J Mol Sci |
| | | 21. |
| ZmWRKY114 | To participate in salt | Bo, C., et al. (2020). Maize WRKY114 gene negatively |
| | stress tolerance | regulates salt-stress tolerance in transgenic rice. Plant Cell |
| | through | Rep 39, 135-148. |
| | ABA-mediated | |
| | pathways | |
| ZmPGIP3 | Disease resistance | Zhu, G., et al. (2019). ZmPGIP3 Gene Encodes a |
| | | Polygalacturonase-Inhibiting Protein that Enhances |
| | | Resistance to Sheath Blight in Rice. Phytopathology 109, |
| | | 1732-1740. |
| ZmGPDH1 | Tolerance to salt | Zhao, Y., et al. (2019). A cytosolic NAD(+)-dependent |
| | and osmotic stress | GPDH from maize (ZmGPDH1) is involved in conferring salt |
| | | and osmotic stress tolerance. BMC Plant Biol 19, 16. |
| ZmDi19-1 | To respond to salt | Zhang, X., et al. (2019). A maize stress-responsive Di19 |
| | stress | transcription factor, ZmDi19-1, confers enhanced tolerance to |
| | | salt in transgenic Arabidopsis. Plant Cell Rep 38, 1563-1578. |
| ZmPORB2 | To increase | Zhan, W., et al. (2019). An allele of ZmPORB2 encoding a |
| | tocopherol | protochlorophyllide oxidoreductase promotes tocopherol |
| | accumulation | accumulation in both leaves and kernels of maize. Plant J |
| | | 100, 114-127. |
| ZmVQ52 | To participate in | Yu, T., et al. (2019). Overexpression of the maize |
| | circadian rhythm | transcription factor ZmVQ52 accelerates leaf senescence in |
| | and photosynthetic | Arabidopsis. PLoS One 14, e0221949. |
| | pathway | |
| ZmAPRG | To increase APA | Yu, T., et al. (2019). ZmAPRG, an uncharacterized gene, |
| | and Pi | enhances acid phosphatase activity and Pi concentration in |
| | concentration in | maize leaf during phosphate starvation. Theor Appl Genet |
| | corn leaves | 132, 1035-1048. |
| ZmEREB180 | Waterlogging | Yu, F., et al. (2019). A group VII ethylene response factor |
| | tolerance | gene, ZmEREB180, coordinates waterlogging tolerance in |
| | | maize seedlings. Plant Biotechnol J 17, 2286-2298. |
| Zmm28 | To improve growth | Wu, J., et al. (2019). Overexpression of zmm28 increases |
| | and photosynthetic | maize grain yield in the field. Proc Natl Acad Sci USA 116, |
| | capacity of corn | 23850-23858. |
| | plants and nitrogen | |
| | use efficiency | |
| ZmDOF36 | To regulate starch | Wu, J., et al. (2019). The DOF-Domain Transcription Factor |
| | synthesis in corn | ZmDOF36 Positively Regulates Starch Synthesis in |
| | endosperm | Transgenic Maize. Front Plant Sci 10, 465. |
| ZmSCE1d | Drought tolerance | Wang, H., et al. (2019). The Maize Class-I SUMO |
| | | Conjugating Enzyme ZmSCE1d Is Involved in Drought |
| | | Stress Response. Int J Mol Sci 21. |
| ZmSCE1e | Stress resistance | Wang, H., et al. (2019). Overexpression of a maize SUMO |
| | | conjugating enzyme gene (ZmSCE1e) increases Sumoylation |
| | | levels and enhances salt and drought tolerance in transgenic |
| | | tobacco. Plant Sci 281, 113-121. |
| ZmGLR | To regulate leaf | Wang, C., et al. (2019). ZmGLR, a cell membrane localized |
| | morphogenesis in | microtubule-associated protein, mediated leaf morphogenesis |
| | corn | in maize. Plant Sci 289, 110248. |
| ZmDEF1 | Resistance to weevil | Vi, T. X. T., et al. (2019). Overexpression of the ZmDEF1 |
| | larvae | gene increases the resistance to weevil larvae in transgenic |
| | | maize seeds. Mol Biol Rep 46, 2177-2185. |
| ZmCCT10 | Corn vegetative and | Stephenson, E., et al. (2019). Over-expression of the |
| | reproductive | photoperiod response regulator ZmCCT10 modifies plant |
| | development | architecture, flowering time and inflorescence morphology in |
| | | maize. PLoS One 14, e0203728. |
| ZmHAK1 | Stress resistance | Qin, Y. J., et al. (2019). ZmHAK5 and ZmHAK1 function in |
| | | K(+) uptake and distribution in maize under low K(+) |
| | | conditions. J Integr Plant Biol 61, 691-705. |
| ZmHAK5 | To enhance K(+) | Qin, Y. J., et al. (2019). ZmHAK5 and ZmHAK1 function in |
| | uptake activity and | K(+) uptake and distribution in maize under low K(+) |
| | promote growth | conditions. J Integr Plant Biol 61, 691-705. |
| ZmNAC34 | Starch synthesis | Peng, X., et al. (2019). A maize NAC transcription factor, |
| | | ZmNAC34, negatively regulates starch synthesis in rice. |
| | | Plant Cell Rep 38, 1473-1484. |
| ZmMYB-IF35 | To increase | Meng, C., et al. (2019). Overexpression of maize MYB-IF35 |
| | resistance to cold | increases chilling tolerance in Arabidopsis. Plant Physiol |
| | and oxidative stress | Biochem 135, 167-173. |
| ZmNAC33 | Drought tolerance | Liu, W., et al. (2019). Function analysis of ZmNAC33, a |
| | | positive regulator in drought stress response in Arabidopsis. |
| | | Plant Physiol Biochem 145, 174-183. |
| ZmRAD51A | Disease resistance | Liu, F., et al. (2019). DNA Repair Gene ZmRAD51A |
| | | Improves Rice and Arabidopsis Resistance to Disease. Int J |
| | | Mol Sci 20. |
| ZmMADS69 | Early flowering | Liang, Y., et al. (2019). ZmMADS69 functions as a flowering |
| | | activator through the ZmRap2.7-ZCN8 regulatory module |
| | | and contributes to maize flowering time adaptation. New |
| | | Phytol 221, 2335-2347. |
| ZmASR3 | Drought tolerance | Liang, Y., et al. (2019). ZmASR3 from the Maize ASR Gene |
| | | Family Positively Regulates Drought Tolerance in |
| | | Transgenic Arabidopsis. Int J Mol Sci 20. |
| ZmPTF1 | Drought tolerance | Li, Z., et al. (2019). The bHLH family member ZmPTF1 |
| | | regulates drought tolerance in maize by promoting root |
| | | development and abscisic acid synthesis. J Exp Bot 70, |
| | | 5471-5486. |
| ZmZIP5 | To play a role in | Li, S., et al. (2019). Improving Zinc and Iron Accumulation |
| | absorption and | in Maize Grains Using the Zinc and Iron Transporter |
| | rhizome | ZmZIP5. Plant Cell Physiol 60, 2077-2085. |
| | transformation of | |
| | zinc and iron | |
| ZmUBP15 | To respond to | Kong, J., et al. (2019). Maize factors ZmUBP15, ZmUBP16 |
| ZmUBP16Z | cadium stress and | and ZmUBP19 play important roles for plants to tolerance the |
| mUBP19 | salt stress | cadmium stress and salt stress. Plant Sci 280, 77-89. |
| ZmCtl1 | To improve stalk | Jiao, S., et al. (2019). Chitinase-likel Plays a Role in Stalk |
| | tensile strength | Tensile Strength in Maize. Plant Physiol 181, 1127-1147. |
| ZmPP2C-A | Drought tolerance | He, Z., et al. (2019). The Maize Clade A PP2C Phosphatases |
| | | Play Critical Roles in Multiple Abiotic Stress Responses. Int |
| | | J Mol Sci 20. |
| ZmPGH1 | To suppress | He, Y., et al. (2019). A maize polygalacturonase functions as |
| | programmed cell | a suppressor of programmed cell death in plants. BMC Plant |
| | death | Biol 19, 310. |
| ZmNAC071 | Stress response | He, L., et al. (2019). Novel Maize NAC Transcriptional |
| | | Repressor ZmNAC071 Confers Enhanced Sensitivity to ABA |
| | | and Osmotic Stress by Downregulating Stress-Responsive |
| | | Genes in Transgenic Arabidopsis. J Agric Food Chem 67, |
| | | 8905-8918. |
| ZmPEPC | To improve carbon | Giuliani, R., et al. (2019). Transgenic maize |
| | metabolism | phosphoenolpyruvate carboxylase alters leaf-atmosphere |
| | | CO2 and (13)CO2 exchanges in Oryza sativa. Photosynth Res |
| | | 142, 153-167. |
| ZmBBM2 | To promote callus | Du, X., et al. (2019). Transcriptome Profiling Predicts New |
| | induction and | Genes to Promote Maize Callus Formation and |
| | transformation | Transformation. Front Plant Sci 10, 1633. |
| ZmbZIP22 | To regulate starch | Dong, Q., et al. (2019). Overexpression of ZmbZIP22 gene |
| | synthesis | alters endosperm starch content and composition in maize |
| | | and rice. Plant Sci 283, 407-415. |
| ZmMADS1a | Positively regulates | Dong, Q., et al. (2019). Functional analysis of ZmMADS1a |
| | starch synthesis | reveals its role in regulating starch biosynthesis in maize |
| | | endosperm. Sci Rep 9, 3253. |
| ZmTCP42 | Drought tolerance | Ding, S., et al. (2019). Genome-Wide Analysis of TCP Family |
| | | Genes in Zea mays L. Identified a Role for ZmTCP42 in |
| | | Drought Tolerance. Int J Mol Sci 20. |
| ATG8 | To obviously | Chen, Q., et al. (2019). Overexpression of ATG8 in |
| | improve nitrogen | Arabidopsis Stimulates Autophagic Activity and Increases |
| | remobilization | Nitrogen Remobilization Efficiency and Grain Filling. Plant |
| | efficiency | Cell Physiol 60, 343-352. |
| SUMO1 | To regulate floral | Chen, J., et al. (2019). Overexpression of SUMO1 located |
| | development | predominately to euchromatin of dividing cells affects |
| | | reproductive development in maize. Plant Signal Behav 14, |
| | | e1588664. |
| ZmMYB167 | To increase biomass | Bhatia, R., et al. (2019). Modified expression ofZmMYB167 |
| | | in Brachypodium distachyon and Zea mays leads to increased |
| | | cell wall lignin and phenolic content. Sci Rep 9, 8800. |
| ZmLEC1 | Fatty acid synthesis | Zhu, Y., et al. (2018). A transgene design for enhancing oil |
| | | content in Arabidopsis and Camelina seeds. Biotechnol |
| | | Biofuels 11, 46. |
| ZmPIP1; 1 | Drought tolerance | Zhou, L., et al. (2018). Overexpression of a maize plasma |
| | and salt stress | membrane intrinsic protein ZmPIP1; 1 confers drought and |
| | | salt tolerance in Arabidopsis. PLoS One 13, e0198639. |
| ZmAIRP4 | Drought tolerance | Yang, L., et al. (2018). Overexpression of the maize E3 |
| | | ubiquitin ligase gene ZmAIRP4 enhances drought stress |
| | | tolerance in Arabidopsis. Plant Physiol Biochem 123, 34-42. |
| ZmNBS42 | Disease resistance | Xu, Y., et al. (2018). Expression of a maize NBS gene |
| | | ZmNBS42 enhances disease resistance in Arabidopsis. Plant |
| | | Cell Rep 37, 1523-1532. |
| ZmNBS25 | Disease resistance | Xu, Y., et al. (2018). The Maize NBS-LRR Gene ZmNBS25 |
| | | Enhances Disease Resistance in Rice and Arabidopsis. Front |
| | | Plant Sci 9, 1033. |
| ZmPt9 | To increase | Xu, Y., et al. (2018). The mycorrhiza-induced maize ZmPt9 |
| | axial root length and | gene affects root development and phosphate availability in |
| | promote lateral root | nonmycorrhizal plant. Plant Signal Behav 13, e1542240. |
| | formation | |
| ZmDA1 | To increase influx | Xie, G., et al. (2018). Over-expression of mutated ZmDA1 or |
| ZmDAR1 | of sugar to organ | ZmDAR1 gene improves maize kernel yield by enhancing |
| | pool from com grain | starch synthesis. Plant Biotechnol J 16, 234-244. |
| | and enhance starch | |
| | synthesis | |
| SAT | To increase | Xiang, X., et al. (2018). Overexpression of serine |
| | prolamine | acetyltransferase in maize leaves increases seed-specific |
| | accumulation | methionine-rich zeins. Plant Biotechnol J 16, 1057-1067. |
| ZmSO | Drought tolerance | Xia, Z., et al. (2018). Overexpression of the Maize Sulfite |
| | | Oxidase Increases Sulfate and GSH Levels and Enhances |
| | | Drought Tolerance in Transgenic Tobacco. Front Plant Sci 9, |
| | | 298. |
| ZmWRKY40 | Drought tolerance | Wang, C. T., et al. (2018). The Maize WRKY Transcription |
| | | Factor ZmWRKY40 Confers Drought Resistance in |
| | | Transgenic Arabidopsis. Int J Mol Sci 19. |
| ZmWRKY106 | To participate in | Wang, C. T., et al. (2018). Maize WRKY Transcription Factor |
| | several stress | ZmWRKY106 Confers Drought and Heat Tolerance in |
| | response pathways | Transgenic Plants. Int J Mol Sci 19. |
| | of abiotic resistance | |
| ZmNF-YB16 | To increase corn | Wang, B., et al. (2018). ZmNF-YB16 Overexpression |
| | yield | Improves Drought Resistance and Yield by Enhancing |
| | | Photosynthesis and the Antioxidant Capacity of Maize Plants. |
| | | Front Plant Sci 9, 709. |
| ZmLAC3 | To increase | Sun, Q., et al. (2018). MicroRNA528 Affects Lodging |
| | lignin content in | Resistance of Maize by Regulating Lignin Biosynthesis under |
| | corn stalk | Nitrogen-Luxury Conditions. Mol Plant 11, 806-814. |
| ZmbZIP4 | To positively | Ma, H., et al. (2018). ZmbZIP4 Contributes to Stress |
| | regulate plant | Resistance in Maize by Regulating ABA Synthesis and Root |
| | abiotic stress | Development. Plant Physiol 178, 753-770. |
| | response and | |
| | participate in corn | |
| | root development | |
| ZmNAGK | Drought tolerance | Liu, W., et al. (2018). Over-Expression of a Maize |
| | | N-Acetylglutamate Kinase Gene (ZmNAGK) Improves |
| | | Drought Tolerance in Tobacco. Front Plant Sci 9, 1902. |
| ZmPIN1a | Form | Li, Z., et al. (2018). Enhancing auxin accumulation in maize |
| | well-developed root | root tips improves root growth and dwarfs plant height. Plant |
| | system to make | Biotechnol J 16, 86-99. |
| | seminal root longer | |
| | and lateral root | |
| | denser | |
| UFGT2 | To increase abiotic | Li, Y. J., et al. (2018). The maize secondary metabolism |
| | stress tolerance of | glycosyltransferase UFGT2 modifies flavonols and |
| | plants | contributes to plant acclimation to abiotic stresses. Ann Bot |
| | | 122, 1203-1217. |
| ZmSRO1b | To enhance | Li, X., et al. (2018). Maize similar to RCD1 gene induced by |
| | resistance to salt | salt enhances Arabidopsis thaliana abiotic stress resistance. |
| | stress, cadmium | Biochem Biophys Res Commun 503, 2625-2632. |
| | stress and oxidative | |
| | stress | |
| ZmDREB4.1 | Development | Li, S., et al. (2018). A DREB-Like Transcription Factor From |
| | regulation | Maize (Zea mays), ZmDREB4.1, Plays a Negative Role in |
| | | Plant Growth and Development. Front Plant Sci 9, 395. |
| Bt2 | To increase seed | Li, N., et al. (2011). “Over-expression of AGPase genes |
| Sh2 | weight and starch | enhances seed weight and starch content in transgenic |
| | content | maize.” Planta 233(2): 241-250. |
| LC | Synthesis of | Le Gall, G., et al. (2003). “Characterization and content of |
| C1 | flavonoid | flavonoid glycosides in genetically modified tomato |
| | | (Lycopersicon esculentum) fruits.” J Agric Food Chem 51(9): |
| | | 2438-2446. |
| ZmbZIP72 | To increase stress | Ying, S., et al. (2012). “Cloning and characterization of a |
| | resistance | maize bZIP transcription factor, ZmbZIP72, confers drought |
| | | and salt tolerance in transgenic Arabidopsis.” Planta 235(2): |
| | | 253-266. |
| ZmCBL4 | Salt tolerance | Wang, M., et al. (2007). “Overexpression of a putative maize |
| | | calcineurin B-like protein in Arabidopsis confers salt |
| | | tolerance.” Plant Mol Biol 65(6): 733-746. |
| ZmCIPK16 | To enhance salt | Zhao, J., et al. (2009). “Cloning and characterization of a |
| | tolerance | novel CBL-interacting protein kinase from maize.” Plant Mol |
| | | Biol 69(6): 661-674. |
| ZmCPK4 | To enhance drought | Jiang, S., et al. (2013). “A maize calcium-dependent protein |
| | tolerance | kinase gene, ZmCPK4, positively regulated abscisic acid |
| | | signaling and enhanced drought stress tolerance in transgenic |
| | | Arabidopsis.” Plant Physiol Biochem 71: 112-120. |
| ZmDREB1A | To enhance drought | Qin, F., et al. (2004). “Cloning and functional analysis of a |
| | tolerance, salt | novel DREB1/CBF transcription factor involved in |
| | tolerance and cold | cold-responsive gene expression in Zea mays L.” Plant Cell |
| | tolerance | Physiol 45(8): 1042-1052. |
| ZmEF-Tu1 | To enhance thermo | Fu, J. and Z. Ristic (2010). “Analysis of transgenic wheat |
| | tolerance | (Triticum aestivum L.) harboring a maize (Zea mays L.) gene |
| | | for plastid EF-Tu: segregation pattern, expression and effects |
| | | of the transgene.” Plant Mol Biol 73(3): 339-347. |
| ZmLEAFY | To increase seed oil | Barthole, G., et al. (2012). “Controlling lipid accumulation in |
| COTYLEDON1 | content | cereal grains.” Plant Sci 185-186: 33-39. |
| ZmWRINKLED1 | | |
| ZmLTP3 | Salt tolerance | Zou, H. W., et al. (2013). “Isolation and Functional Analysis |
| | | of ZmLTP3, a Homologue to Arabidopsis LTP3.” Int J Mol |
| | | Sci 14(3): 5025-5035. |
| ZmMKK4 | Salt and cold | Kong, X., et al. (2011). “ZmMKK4, a novel group C |
| | tolerance | mitogen-activated protein kinase kinase in maize (Zea mays), |
| | | confers salt and cold tolerance in transgenic Arabidopsis.” |
| | | Plant Cell Environ 34(8): 1291-1303. |
| ZmPEAMT1 | To promote root | Wu, S., et al. (2007). “Cloning, characterization, and |
| | growth and enhance | transformation of the phosphoethanolamine |
| | salt tolerance | N-methyltransferase gene (ZmPEAMT1) in maize (Zea mays |
| | | L.).” Mol Biotechnol 36(2): 102-112. |
| ZmPIS | Drought tolerance | Zhai, S. M., et al. (2012). “Overexpression of the |
| | | phosphatidylinositol synthase gene from Zea mays in tobacco |
| | | plants alters the membrane lipids composition and improves |
| | | drought stress tolerance.” Planta 235(1): 69-84 |
| ZmPP2C2 | Cold tolerance | Hu, X., et al. (2010). “Enhanced tolerance to low temperature |
| | | in tobacco by over-expression of a new maize protein |
| | | phosphatase 2C, ZmPP2C2.” J Plant Physiol 167(15): |
| | | 1307-1315. |
| ZmRFP1 | Drought tolerance | Liu, J., et al. (2013). “Overexpression of a maize E3 ubiquitin |
| | | ligase gene enhances drought tolerance through regulating |
| | | stomatai aperture and antioxidant system in transgenic |
| | | tobacco.” Plant Physiol Biochem 73: 114-120. |
| ZmSAPK8 | Salt tolerance | Ying, S., et al. (2011). “Cloning and characterization of a |
| | | maize SnRK2 protein kinase gene confers enhanced salt |
| | | tolerance in transgenic Arabidopsis.” Plant Cell Rep 30(9): |
| | | 1683-1699. |
| ZmSIMK1 | Salt tolerance | Gu, L., et al. (2010). “Overexpression of maize |
| | | mitogen-activated protein kinase gene, ZmSIMK1 in |
| | | Arabidopsis increases tolerance to salt stress.” Mol Biol Rep |
| | | 37(8): 4067-4073. |
| ZmLEC1 | To increase seed oil | Shen, B., et al. (2010). “Expression of ZmLEC1 and |
| ZmWRI1 | content | ZmWRI1 increases seed oil production in maize.” Plant |
| | | Physiol 153(3): 980-987. |
| ZmWrinkled1 | To increase seed oil | Pouvreau, B., et al. (2011). “Duplicate maize Wrinkled1 |
| | content | transcription factors activate target genes involved in seed oil |
| | | biosynthesis.” Plant Physiol 156(2): 674-686. |

Table G lists the representative functional genes in barley. Methods in the present invention can be used to edit such genes and create new genes by designing new combinations of different gene elements or different protein domain, and can be utilized in barley breeding program.

TABLE G
Important functional genes in barley

| Gene name | Application | Reference |
|---|---|---|
| HGGT | Grain size and | Chen, J., et al. (2017). Overexpression of hvhggt enhances |
| (LOC548177) | weight | tocotrienol levels and antioxidant activity in barley. J. Agric. |
| | | Food Chem.. |
| HvSERK2 | Resistance to | Liu,Y. B., et al. (2018). Transient overexpression of hvserk2 |
| | powdery mildew | improves barley resistance to powdery mildew. International |
| | | Journal of Molecular Sciences, 19(4), 1226. |
| HvAKT1 | Drought | Feng, X., et al. (2020). Overexpression of hvakt1 improves |
| | tolerance | barley drought tolerance by regulating root ion homeostasis |
| | | and ros and no signaling. Journal of Experimental Botany. |
| HvADH-1 | Disease | Kasbauer Christoph L, et al. (2017). Barley ADH-1 modulates |
| (LOC548236) | resistance | susceptibility to Bgh and is involved in chitin-induced |
| | | systemic resistance. Plant Physiology and Biochemistry. |
| HvOS2 | To delay | Greenup, A. G., et al. (2010). Oddsoc2 is a MADS box floral |
| | blooming | repressor that is down-regulated by vernalization in temperate |
| | | cereals. Plant physiology, 153(3), 1062-1073. |
| HvCO1/ | Vernalization | Mulki M A., Korff M V. (2015). Constans controls floral |
| HvFT1 | regulation | repression by upregulating vernalization 2 (vrn-h2) in barley. |
| | | Plant Physiology, 170(1), 325. |
| Hvhak1 | Drought | Feng, X., et al. (2020). Hvakt2 and hvhak1 confer drought |
| | tolerance | tolerance in barley through enhanced leaf mesophyl1 h+ |
| | | homoeostasis. Wiley-Blackwell Online Open, 18(8), 1683. |
| DREB1 | Stress resistance | Xu, Z. S., et al. (2009). Isolation and functional |
| | | characterization of hvdreb1-a gene encoding a |
| | | dehydration-responsive element binding protein in hordeum |
| | | vulgare. Journal of Plant Research, 122(1), 121-130. |
| CslF6 | Yield increase | Lim, W. L., et al. (2019). Overexpression of hvcslf6 in barley |
| | | grain alters carbohydrate partitioning plus transfer tissue and |
| | | endosperm development. Journal of Experimental Botany, |
| | | 71(1). |
| HvPIP2; 3/ | Salt tolerance | Lim, W. L., et al. (2019). Overexpression of hvcslf6 in barley |
| HvPIP2; 4/ | | grain alters carbohydrate partitioning plus transfer tissue and |
| HvPIP2; 1 | | endosperm development. Journal of Experimental Botany, |
| | | 71(1). |
| HvNAS1 | To increase zinc | Hiroshi, Masuda., et al. (2009). Overexpression of the Barley |
| | and iron content | Nicotianamine Synthase GeneHvNAS1Increases Iron and Zinc |
| | in grains | Concentrations in Rice Grains., 2(4), 155-166. |
| NHX2 | Salt tolerance | Bayat F, et al. (2011). Overexpression of hvnhx2, a vacuolar |
| | | na+/h+ antiporter gene from barley, improves salt tolerance in |
| | | ‘arabidopsis thaliana’. Australian Journal of Crop Science, |
| | | 5(4), 428-432. |
| VRN1 | Vernalization | Daniel P Woods, et al. (2016). Evolution of vrn2/ghd7-like |
| | regulation | genes in vernalization-mediated repression of grass flowering. |
| | | Plant Physiology, 170 (4), 2124-2135. |

Table H lists the representative functional genes in rice. Methods in the present invention can be used to edit such genes and create new genes by designing new combinations of different gene elements or different protein domain, and can be utilized in rice breeding program.

TABLE H
Important functional genes in rice

| Gene name | Application | Reference |
|---|---|---|
| OsATG8b | Grain quality | Fan, T., et al. (2020). “A Rice Autophagy Gene |
| | | OsATG8b Is Involved in Nitrogen Remobilization and |
| | | Control of Grain Quality.” Front Plant Sci 11: 588. |
| OsGsa1 | To increase grain size | Dong, N. Q., et al. (2020). “UDP-glucosyltransferase |
| | and enhance abiotic | regulates grain size and abiotic stress tolerance |
| | stress tolerance | associated with metabolic flux redirection in rice.” |
| | | Nat Commun 11(1): 2629. |
| OsI-BAK1 | Grain filling and leaf | Khew, C. Y., et al. (2015). “Brassinosteroid |
| | development | insensitive 1-associated kinase 1 (OsI-BAK1) is |
| | | associated with grain filling and leaf development in |
| | | rice.” J Plant Physiol 182: 23-32. |
| OsCATA | Grain development | Suppression of phospholipase D genes improves |
| | | chalky grain production by |
| | | high temperature during the grain-filling stage in rice |
| OsGRF4 | Grain size and yield | Duan, P., et al. (2015). “Regulation of OsGRF4 by |
| | | OsmiR396 controls grain size and yield in rice.” Nat |
| | | Plants 2: 15203. |
| small grain | Grain yield | Fang, N., et al. (2016). “SMALL GRAIN 11 Controls |
| D2/SMG11 | | Grain Size, Grain Number and Grain Yield in Rice.” |
| | | Rice (NY) 9(1): 64. |
| OsHk6 | Grain yield | Choi, J., et al. (2012). “Functional identification of |
| | | OsHk6 as a homotypic cytokinin receptor in rice with |
| | | preferential affinity for iP.” Plant Cell Physiol 53(7): |
| | | 1334-1343. |
| OsRAA1 | Growth of axial root | Han, Y., et al. (2005). “Biochemical character of the |
| | and lateral root | purified OsRAA1, a novel rice protein with |
| | | GTP-binding activity, and its expression pattern in |
| | | Oryza sativa.” J Plant Physiol 162(9): 1057-1063. |
| OsHDAC1 | Plant architecture | Jang, I. C., et al. (2003). “Structure and expression of |
| | | the rice class-I type histone deacetylase genes |
| | | OsHDAC1-3: OsHDAC1 overexpression in transgenic |
| | | plants leads to increased growth rate and altered |
| | | architecture.” Plant J 33(3): 531-541. |
| OsbHLH073 | Plant architecture | Lee, J., et al. (2020). “OsbHLH073 Negatively |
| | | Regulates Internode Elongation and Plant Height by |
| | | Modulating GA Homeostasis in Rice.” Plants (Basel) |
| | | 9(4). |
| OsBAK1 | Plant architecture | Li, D., et al. (2009). “Engineering OsBAK1 gene as a |
| | | molecular tool to improve rice architecture for high |
| | | yield.” Plant Biotechnol J 7(8): 791-806. |
| TIFY11b | Plant height and grain | Hakata, M., et al. (2012). “Overexpression of a rice |
| | size | TIFY gene increases grain size through enhanced |
| | | accumulation of carbohydrates in the stem.” Biosci |
| | | Biotechnol Biochem 76(11): 2129-2134. |
| OsPSK3 | Plant height and | Huang, J. Y., et al. (2010). “[Over-expression of |
| | chlorophyll content | OsPSK3 increases chlorophyll content of leaves in |
| | | rice].” Yi Chuan 32(12): 1281-1289. |
| CYP94 | Plant height | Kurotani, K. I., et al. (2015). “Overexpression of a |
| | | CYP94 family gene CYP94C2b increases internode |
| | | length and plant height in rice.” Plant Signal Behav |
| | | 10(7): e1046667. |
| OsRSR1 | Seed quality and yield | Fu, F. F., et al. (2010). “Coexpression analysis |
| | | identifies Rice Starch Regulator1, a rice AP2/EREBP |
| | | family transcription factor, as a novel rice starch |
| | | biosynthesis regulator.” Plant Physiol 154(2): |
| | | 927-938. |
| RPBF | Seed quality | Yamamoto, M. P., et al. (2006). “Synergism between |
| | | RPBF Dof and RISBZ1bZIP Activators in the |
| | | Regulation of Rice SeedExpression Genes.” Plant |
| | | Physiol |
| PDI | Seed storage proteins | Hiroshi, Yasuda., et al. (2009). “Overexpression of BiP |
| | | has Inhibitory Effects on theAccumulation of Seed |
| | | Storage Proteins in EndospermCells of Rice.” Plant & |
| | | Cell Physiology, 50(8), 1532. |
| BiP | Seed storage proteins | Hiroshi, Yasuda., et al. (2009). “Overexpression of |
| | | BiP has Inhibitory Effects on the Accumulation of |
| | | Seed Storage Proteins in Endosperm Cells of Rice.” |
| | | Plant & Cell Physiology, 50(8), 1532. |
| OsWRKY22 | To positively regulate | Ge, Z. L., et al. (2018). “Transcription factor |
| | tolerance to aluminum | WRKY22 promotes aluminum tolerance viaactivation |
| | | of OSFRDL4 expression and enhancement of citrate |
| | | secretion in rice (oryza sativa)”. New Phytologist, |
| | | 219. |
| OsDof12 | To affect flowering | Li, D., et al. (2009). “Functional characterization of |
| | under long day length | rice OsDof12.” Planta 229(6): 1159-1169. |
| | conditions | |
| BSR1 | To enhance immune | Kanda, Y., et al. (2019). “Broad-Spectrum Disease |
| | response | Resistance Conferred by the Overexpression of Rice |
| | | RLCK BSR1 Results from an Enhanced Immune |
| | | Response to Multiple MAMPs.” Int J Mol Sci 20(22). |
| OsTLP27 | To increase | Hu, F., et al. (2012). “Overexpression of OsTLP27 in |
| | photosynthesis | rice improves chloroplast function and photochemical |
| | | efficiency.” Plant Sci 195: 125-134. |
| OsSRT1 | To enhance tolerance | Huang, L., et al. (2007). “Down-regulation of a |
| | to oxidative | SILENT INFORMATION REGULATOR2-related |
| | responsive stress | histone deacetylase gene, OsSRT1, induces DNA |
| | | fragmentation and cell death in rice.” Plant Physiol |
| | | 144(3): 1508-1519. |
| LSCHL4 | To increase yield | Zhang, G. H., et al. “LSCHL4 from Japonica Cultivar, |
| | | Which Is Allelic to NAL1, Increases Yield of Indica |
| | | Super Rice 93-11.” Molecular Plant(8), 1350-1364. |
| OsNRT2.1 | To increase yield and | Luo, B., et al. (2018). “Overexpression of a |
| | weight | High-Affinity Nitrate Transporter OsNRT2.1 |
| | | Increases Yield and Manganese Accumulation in Rice |
| | | UnderAlternating Wet and Dry Condition.” Frontiers |
| | | in Plant ence, 9, 1192. |
| GABA | To maintain iron | Zhu, C., et al. (2020). “γ-Aminobutyric Acid |
| | homeostasis in rice | Suppresses Iron Transportation from Roots to Shoots |
| | seedlings | in Rice Seedlings by InducingAerenchyma |
| | | Formation.” International Journal of Molecular |
| | | Sciences, 22(1), 220. |
| Roc5 | Leaf shape | Zou, L. P., et al. (2011). “Leaf rolling controlled by |
| | | the homeodomain leucine zipper class IV gene Roc5 |
| | | in rice.” Plant Physiol 156(3): 1589-1602. |
| OsPCF8 | Leaf morphogenesis | Yang, C., et al.(2013). “Overexpression of |
| | and cold tolerance | microRNA319 impacts leaf morphogenesis and leads |
| | | to enhanced cold tolerance in rice (Oryza sativaL.).” |
| | | Plant Cell & Environment, 36(12). |
| OsPCF5 | Leaf morphogenesis | Yang, C., et al. (2013). “Overexpression of |
| | and cold tolerance | microRNA319 impacts leaf morphogenesis and leads |
| | | to enhanced cold tolerance in rice (Oryza sativaL.)”. |
| | | Plant Cell & Environment, 36(12). |
| Osa-MIR319a | Leaf morphogenesis | Yang, C., et al. (2013). “Overexpression of |
| | and cold tolerance | microRNA319 impacts leaf morphogenesis and leads |
| | | to enhanced cold tolerance in rice (Oryza sativaL.).” |
| | | Plant Cell & Environment, 36(12). |
| OsClpP6 | Leaf senescence | Zhao, X., et al. (2021). “OsNBL1, a Multi-Organelle |
| | | Localized Protein, Plays Essential Roles in Rice |
| | | Senescence, Disease Resistance, and Salt |
| | | Tolerance.” Rice, 14(1). |
| RAV6 | Leaf angle and seed | Zhang, XQ., et al. (2015). Epigenetic mutation of |
| | size | RAV6 affects leaf angle and seed size in rice. PLANT |
| | | PHYSIOL, 2015, 169(3), 2118-2128. |
| OsHDY1 | Chloroplast | Zhao, J., et al. (2015). Functional inactivation of |
| | development | putative photosynthetic electron acceptor ferredoxin |
| | | c2 (fdc2) induces delayed heading date and decreased |
| | | photosynthetic rate in rice. Pios One, 10. |
| OsTRM13 | Salt stress tolerance | Youmei, Wang., et al. (2017). The |
| | | 2′-o-methyladenosine nucleoside modification gene |
| | | ostrml3 positively regulates salt stress tolerance in |
| | | rice. Journal of Experimental Botany. |
| OsHKT2; 4 | Salt balance | Chi, Zhang., et al. (2017). The rice high-affinity k+ |
| | | transporter OsHKT2; 4 mediates Mg2+ homeostasis |
| | | under high-Mg2+ conditions in transgenic |
| | | arabidopsis. Frontiers in Plant Science, 8. |
| OsXDH | To delay leaf | Han, R., et al. (2020). “Enhancing xanthine |
| | senescence and | dehydrogenase activity is an effective way to delay |
| | increase rice yield | leaf senescence and increase rice yield.” Rice (NY) |
| | | 13(1): 16. |
| TDC | To delay leaf | Kang, K., et al. (2009). “Senescence-induced |
| | senescence | serotonin biosynthesis and its role in delaying |
| | | senescence in rice leaves.” Plant Physiol 150(3): |
| | | 1380-1393. |
| OsDOS | To delay leaf | Kong, Z., et al. (2006). “A novel nuclear-localized |
| | senescence | CCCH-type zinc finger protein, OsDOS, is involved |
| | | in delaying leaf senescence in rice.” Plant Physiol |
| | | 141(4): 1376-1388. |
| bHLH142 | Male sterility | Ko, S. S., et al. (2017). “Tightly Controlled |
| | | Expression of bHLH142 Is Essential for Timely |
| | | Tapetai Programmed Cell Death and Pollen |
| | | Development in Rice.” Front Plant Sci 8: 1258. |
| OsZIP8 | Zinc uptake and | Lee, S., et al. (2010). “Zinc deficiency-inducible |
| | distribution | OsZIP8 encodes a plasma membrane-localized zinc |
| | | transporter in rice.” Mol Cells 29(6): 551-558. |
| OsZIP5 | Zinc distribution | Lee, S., et al. (2010). “OsZIP5 is a plasma membrane |
| | | zinc transporter in rice.” Plant Mol Biol 73(4-5): |
| | | 507-517. |
| OsZIP4 | Zinc distribution | Ishimaru, Y., et al. (2007). “Overexpression of the |
| | | OsZIP4 zinc transporter confers disarrangement of |
| | | zinc distribution in rice plants.” J Exp Bot 58(11): |
| | | 2909-2915. |
| APO1 | Spikelet number | Ikeda, K., et al. (2007). “Rice ABERRANT PANICLE |
| | | ORGANIZATION 1, encoding an F-box protein, |
| | | regulates meristem fate.” Plant J 51(6): 1030-1040. |
| OsPGIP4 | Resistance to bacterial | Feng, C., et al. (2016). “The |
| | leaf streak in rice | polygalacturonase-inhibiting protein 4 (OsPGIP4), a |
| | | potential component of the qBlsr5a locus, confers |
| | | resistance to bacterial leaf streak in rice.” Planta |
| | | 243(5): 1297-1308. |
| OsGAP1 | Resistance to bacterial | Cheung, M. Y., et al. (2008). “Constitutive expression |
| | pathogen | of a rice GTPase-activating protein induces defense |
| | | responses.” New Phytol 179(2): 530-545. |
| BBM1 | Vegetative | Khanday, I., et al. (2019). “A male-expressed rice |
| | propagation | embryogenic trigger redirected for asexual |
| | | propagation through seeds.” Nature 565(7737): 91-95. |
| OsGSTU5 | Resistance to sheath | Tiwari, M., et al. (2020). “Functional characterization |
| | blight disease | of tau class glutathione-S-transferase in rice to |
| | | provide tolerance against sheath blight disease.” 3 |
| | | Biotech 10(3): 84. |
| OsPGIP1 | Resistance to sheath | Chen, X. J., et al. (2016). “Overexpression of |
| | blight disease | OsPGIP1 Enhances Rice Resistance to Sheath Blight.” |
| | | Plant Dis 100(2): 388-395. |
| RGG1 and | Sheath blight disease | Swain, D. M., et al. (2019). “Concurrent |
| RGB1 | | overexpression of rice G-protein beta and gamma |
| | | subunits provide enhanced tolerance to sheath blight |
| | | disease and abiotic stress in rice.” Planta 250(5): |
| | | 1505-1520. |
| OsMYB4 | Sheath blight disease | Pooja, S., et al. (2015). “Homotypic clustering of |
| | | OsMYB4 binding site motifs in promoters of the rice |
| | | genome and cellular-level implications on sheath |
| | | blight disease resistance.” Gene 561(2): 209-218. |
| OsNLA1 | To maintain phosphate | Yue, W., et al. (2017). Osnla1, a ring-type ubiquitin |
| | homeostasis | ligase, maintains phosphate homeostasis in oryza |
| | | sativa via degradation of phosphate transporters. The |
| | | Plant Journal, 90(6), 1040. |
| OsYSL15 | Iron uptake | Lee, S., et al. (2009). “Disruption of OsYSL15 leads |
| | | to iron inefficiency in rice plants.” Plant Physiol |
| | | 150(2): 786-800. |
| OsYSL13 | Iron distribution | Chang., et al. (2018). OsYSL13 is involved in iron |
| | | distribution in rice. International Journal of Molecular |
| | | Sciences. |
| OsIRT1 | Iron and zinc uptake | Lee, S. and G. An (2009). “Over-expression of |
| | | OsIRT1 leads to increased iron and zinc |
| | | accumulations in rice.” Plant Cell Environ 32(4): |
| | | 408-416. |
| OsVIT2 | Iron and zinc | Zhang, Y., et al.(2012). Vacuolar membrane |
| | translocation | transporters osvit1 and osvit2 modulate iron |
| | | translocation between flag leaves and seeds in rice. |
| | | Plant Journal for Cell & Molecular Biology, 72(3), |
| | | 400-410. |
| OsVIT1 | Iron and zinc | Zhang, Y., et al.(2012). Vacuolar membrane |
| | translocation | transporters osvit1 and osvit2 modulate iron |
| | | translocation between flag leaves and seeds in rice. |
| | | Plant Journal for Cell & Molecular Biology, 72(3), |
| | | 400-410. |
| OsYSL2 | Iron and manganese | Ishimaru, Y., et al. (2010). “Rice metal-nicotianamine |
| | uptake | transporter, OsYSL2, is required for the long-distance |
| | | transport of iron and manganese.” Plant J 62(3): |
| | | 379-390. |
| OsGSTU12 | To regulate leaf | Zhao, N., et al.(2020). Over-expression of HDA710 |
| | senescence | delays leaf senescence in rice (oryza sativa l.). |
| | | Frontiers in Bioengineering and Biotechnology, 8, |
| | | 471. |
| HDA710 | To regulate leaf | Zhao, N., et al.(2020). Over-expression of HDA10 |
| | senescence | delays leaf senescence in rice (oryza sativa l.). |
| | | Frontiers in Bioengineering and Biotechnology, 8, |
| | | 471. |
| OsDof4 | To regulate flowering | Qi, W., et al. (2017). Constitutive expression of |
| | period | osdof4, encoding a c2-c2 zinc finger transcription |
| | | factor, confesses its distinct flowering effects under |
| | | long- and short-day photoperiods in rice (oryza sativa |
| | | l.). BMC Plant Biology, 17. |
| RFT1 | To regulate flowering | Lichao Zhang., et al.(2016). The wheat MYB-related |
| | time | transcription factor TaMYB72 promotes flowering in |
| | | rice. Journal of Integrative Plant Biology, 08(v.58), |
| | | 7-10. |
| Hd3a | To regulate flowering | Lichao Zhang., et al.(2016). The wheat MYB-related |
| | time | transcription factor TaMYB72 promotes flowering in |
| | | rice. Journal of Integrative Plant Biology, 08(v.58), |
| | | 7-10. |
| OsLAC13 | To regulate seed | Yu, Y., et al. (2017). Laccase-13 regulates seed |
| | setting rate | setting rate by affecting hydrogen peroxide dynamics |
| | | and mitochondrial integrity in rice. Frontiers in Plant |
| | | Science, 8. |
| OsHXK1 | To regulate | Zheng, S., et al. (2019). Pnas plus: osago2 controls ros |
| | anther development | production and the initiation of tapetai ped by |
| | | epigenetically regulating oshxk1 expression in rice |
| | | anthers. Proceedings of the National Academy of |
| | | Sciences of the United States of America, 116(15). |
| OsAGO2 | To regulate | Zheng, S., et al. (2019). Pnas plus: osago2 controls ros |
| | anther development | production and the initiation of tapetai ped by |
| | | epigenetically regulating oshxk1 expression in rice |
| | | anthers. Proceedings of the National Academy of |
| | | Sciences of the United States of America, 116(15). |
| TAWAWA1 | To regulate | Yoshida, A., et al (2013). TAWAWA1, a regulator |
| | growth development | of rice inflorescence architecture, functions through |
| | | the suppression of meristem phase transition. |
| | | Proceedings of the National Academy of Sciences, |
| | | 110(2), 767-772. |
| MAIF1 | To regulate root | C, Yong., et al. (2010). Overexpression of an f-box |
| | growth | protein gene reduces abiotic stress tolerance and |
| | | promotes root growth in rice. Molecular Plant, 4(1), |
| | | 190-197. |
| osa-miR171b | Stripe virus | Tong, A., et al. (2017). “Altered accumulation of |
| | | osa-miR171b contributes to rice stripe virus infection |
| | | by regulating disease symptoms.” J Exp Bot 68(15): |
| | | 4357-4367. |
| OsSERK1 | To generate somatic | Hu, H., et al. (2005). “Rice SERK1 gene positively |
| | embryogenesis and | regulates somatic embryogenesis of cultured cell and |
| | enhance rice blast | host defense response against fungal infection.” Planta |
| | resistance | 222(1): 107-117. |
| OsNRT1.1A | To improve crop yield | Wang, W., et al. (2018). “Expression of the Nitrate |
| | and shorten crop | Transporter Gene OsNRT1.1A/OsNPF6.3 Confers |
| | maturation | High Yield and Early Maturation in Rice.” Plant Cell |
| | | 30(3): 638-651. |
| OsPT8 | To improve inorganic | Jia, H., et al. (2011). “The phosphate transporter gene |
| | phosphate uptake | OsPht1; 8 is involved in phosphate homeostasis in |
| | | rice.” Plant Physiol 156(3): 1164-1175. |
| OsDRF1 | To enhance disease | Cao, Y., et al. (2008). “Overexpression of a rice |
| | resistance (mosaic | defense-related F-box protein gene OsDRFl in |
| | virus and | tobacco improves disease resistance through |
| | pseudomonas) | potentiation of defense gene expression.” Physiol |
| | | Plant 134(3): 440-452. |
| PHD1 | To increase | Li, C., et al. (2011). “A rice plastidial nucleotide sugar |
| | photosynthetic | epimerase is involved in galactolipid biosynthesis and |
| | efficiency and crop | improves photosynthetic efficiency.” PLoS Genet |
| | yield | 7(7): e1002196. |
| OsIRL | To improve tolerance | Kim, S. G., et al. (2010). “Overexpression of rice |
| | to peroxides | isoflavone reductase-like gene (OsIRL) confers |
| | | tolerance to reactive oxygen species.” Physiol Plant |
| | | 138(1): 1-9. |
| RAG2 | To increase yield and | “Overexpression of the 16-kDa a-amylase/trypsin |
| | quality | inhibitor RAG2 improves grain yield and quality of |
| | | rice” |
| OsAT10 | To enhance | Li, G., et al. (2018). “Overexpression of a rice BAHD |
| | saccharification | acyltransferase gene in switchgrass (Panicum |
| | | virgatum L.) enhances saccharification.” BMC |
| | | Biotechnol 18(1): 54. |
| OsSec18 | To increase rice plant | Sun, Y., et al. (2015). “The OsSec18 complex |
| | height and thousand | interacts with P0(P1-P2)2 to regulate vacuolar |
| | kernel weight | morphology in rice endosperm cell.” BMC Plant Biol |
| | | 15: 55. |
| OsPGIP2 | To enhance sheath | Chen, X., et al. (2019). “Amino acid substitutions in a |
| | blight resistance in | polygalacturonase inhibiting protein (OsPGIP2) |
| | rice | increases sheath blight resistance in rice.” Rice (NY) |
| | | 12(1): 56. |
| OsWRKY4 | Sheath blight | Wang, H., et al. (2015). “Rice WRKY4 acts as a |
| | resistance in rice | transcriptional activator mediating defense responses |
| | | toward Rhizoctonia solani, the causing agent of rice |
| | | sheath blight.” Plant Mol Biol 89(1-2): 157-171. |
| Pti1a | Rice resistance | Takahashi, A., et al. (2007). “Rice Pti1a negatively |
| | | regulates RAR1-dependent defense responses.” Plant |
| | | Cell 19(9): 2940-2951. |
| OsCDC48 | Rice resistance | Shi, L., et al. (2019). “OsCDC48/48E complex is |
| | | required for plant survival in rice (Oryza sativa L.).” |
| | | Plant Mol Biol 100(1-2): 163-179. |
| OsFLS2 | Bacteria resistance in | Wang, S., et al. (2015). “Rice OsFLS2-Mediated |
| | rice | Perception of Bacterial Flagellins Is Evaded by |
| | | Xanthomonas oryzae pvs. oryzae and oryzicola.” Mol |
| | | Plant 8(7): 1024-1037. |
| SNAC3 | Drought tolerant and | Fang, Y., et al. (2015). “A stress-responsive NAC |
| | heat resistant gene in | transcription factor SNAC3 confers heat and drought |
| | rice | tolerance through modulation of reactive oxygen |
| | | species in rice.” J Exp Bot 66(21): 6803-6817. |
| KNAT7 | Lodging resistance | Wang, S., et al. (2019). “Rice Homeobox Protein |
| | and yield of rice | KNAT7 Integrates the Pathways Regulating Cell |
| | | Expansion and Wall Stiffness.” Plant Physiol 181(2): |
| | | 669-682. |
| OsRLR1&OSWRKY19 | Disease resistance in | Du, D., et al. (2020). “The CC-NB-LRR OsRLR1 |
| | rice | mediates rice disease resistance through interaction |
| | | with OsWRKY19.” Plant Biotechnol J. |
| OsCDR1 | Disease resistance and | Prasad, B. D., et al. (2009). “Overexpression of rice |
| | defensive mechanism | (Oryza sativa L.) OsCDR1 leads to constitutive |
| | of rice | activation of defense responses in rice and |
| | | Arabidopsis.” Mol Plant Microbe Interact 22(12): |
| | | 1635-1644. |
| OsRDR1 | Virus resistance in | Wang, H., et al. (2016). “A Signaling Cascade from |
| | rice | miR444 to RDR1 in Rice Antiviral RNA Silencing |
| | | Pathway.” Plant Physiol 170(4): 2365-2377. |
| OsWRKY13 | Disease resistance in | Qiu, D., et al. (2007). “OsWRKY13 mediates rice |
| | rice | disease resistance by regulating defense-related genes |
| | | in salicylate- and jasmonate-dependent signaling.” |
| | | Mol Plant Microbe Interact 20(5): 492-499. |
| GIF1 | Rice grain-filling and | Wang, E., et al. (2008). “Control of rice grain-filling |
| | yield | and yield by a gene with a potential signature of |
| | | domestication.” Nat Genet 40(11): 1370-1374. |
| OsAAP5 | Rice tiller number and | Wang, J., et al. (2019). “The Amino Acid Permease 5 |
| | yield | (OsAAP5) Regulates Tiller Number and Grain Yield |
| | | in Rice.” Plant Physiol 180(2): 1031-1045. |
| OsRIP1 | Rice development | Wytynck, P., et al.(2021). Effect of rip overexpression |
| | | on abiotic stress tolerance and development of rice. |
| | | International Journal of Molecular Sciences, 22(3), |
| | | 1434. |
| OsmiR156b | Rice development | Kabin Xie., et al. (2006). Genomic organization, |
| | | differential expression, and interaction of squamosa |
| | | promoter-binding-like transcription factors and |
| | | microrna156 in rice. Plant Physiology, 142(1), |
| | | 280-93. |
| OsMADS57 | Rice development | Yin, X., et al. (2019). OsMADS18, a |
| | | membrane-bound MADS-box transcription factor, |
| | | modulates plant architecture and the abscisic acid |
| | | response in rice. Journal of Experimental Botany(15), |
| | | 3895-3909. |
| OsMADS18 | Rice development | Yin, X., et al. (2019). OsMADS18, a |
| | | membrane-bound MADS-box transcription factor, |
| | | modulates plant architecture and the abscisic acid |
| | | response in rice. Journal of Experimental Botany(15), |
| | | 3895-3909. |
| OsMADS15 | Rice development | Yin, X., et al. (2019). OsMADS18, a |
| | | membrane-bound MADS-box transcription factor, |
| | | modulates plant architecture and the abscisic acid |
| | | response in rice. Journal of Experimental Botany(15), |
| | | 3895-3909. |
| OsMADS14 | Rice development | Yin, X., et al. (2019). OsMADS18, a |
| | | membrane-bound MADS-box transcription factor, |
| | | modulates plant architecture and the abscisic acid |
| | | response in rice. Journal of Experimental Botany(15), |
| | | 3895-3909. |
| NF-YC12 | Rice development | Xiong, Y., et al. (2019). NF-YC12 is a key |
| | | multi-functional regulator of accumulation of seed |
| | | storage substances in rice. Journal of Experimental |
| | | Botany(15), 15. |
| CCP1 | Rice development | Yan, D., et al. (2015). Curved chimeric palea 1 |
| | | encoding an EMF1-like protein maintains epigenetic |
| | | repression of OsMADS58 in rice palea development. |
| | | Plant Journal, 82(1), 12-24. |
| LPA1 | Resistance to sheath | Sun, Q., et al. (2020). “Indeterminate Domain Proteins |
| | blight disease in rice | Regulate Rice Defense to Sheath Blight Disease.” |
| | | Rice (NY) 13(1): 15. |
| EPSPS | To improve | Achary, V. M. M., et al. (2020). “Overexpression of |
| | glyphosate tolerance | improved EPSPS gene results in field level glyphosate |
| | and increase grain | tolerance and higher grain yield in rice.” Plant |
| | yield in rice | Biotechnol J 18(12): 2504-2519. |
| Dehydroascorbate | Rice yield and | Do, H., et al. (2016). “Structural understanding of the |
| reductase | biomass | recycling of oxidized ascorbate by dehydroascorbate |
| OsDHAR | | reductase (OsDHAR) from Oryza sativa L. japonica.” |
| | | Sci Rep 6: 19498. |
| OsECS(gamma-ecs) | Rice yield and its | Choe, Y. H., et al. (2013). “Homologous expression of |
| | tolerance to | gamma-glutamylcysteine synthetase increases grain |
| | environmental stresses | yield and tolerance of transgenic rice plants to |
| | | environmental stresses.” J Plant Physiol 170(6): |
| | | 610-618. |
| WRKY45 | Rice blast resistance | Ueno, Y., et al. (2017). “WRKY45 phosphorylation at |
| | | threonine 266 acts negatively on WRKY45-dependent |
| | | blast resistance in rice.” Plant Signal Behav 12(8): |
| | | e1356968. |
| Pikh Gene | Rice blast resistance | Azizi, P., et al. (2016). “Over-Expression of the Pikh |
| | | Gene with a CaMV 35S Promoter Leads to Improved |
| | | Blast Disease (Magnaporthe oryzae) Tolerance in |
| | | Rice.” Front Plant Sci 7: 773. |
| EcGDH | To improve nitrogen | Tang, D., et al. (2018). “Ectopic expression of fungal |
| | assimilation and grain | EcGDH improves nitrogen assimilation and grain |
| | yield in rice | yield in rice.” J Integr Plant Biol 60(2): 85-88. |
| NADH-GOGAT | To improve nitrogen | Tomoyuki Yamaya1, 2, 4, Mitsuhiro Obara1, Hiroyuki |
| | utilization and grain | Nakajima1, Shohei Sasaki1, |
| | filling in rice | Toshihiko Hayakawa1 and Tadashi Sato3 |
| Fie1 | Rice yield (grain size) | Dhatt, B. K., et al. (2021). “Allelic variation in rice |
| | | Fertilization Independent Endosperm 1 contributes to |
| | | grain width under high night temperature stress.” New |
| | | Phytol 229(1): 335-350. |
| OsMYB1R1-VP64 | Rice yield | Wang, J., et al. (2016). “Overexpression of |
| | | OsMYB1R1-VP64 fusion protein increases grain yield |
| | | in rice by delaying flowering time.” FEBS Lett |
| | | 590(19): 3385-3396. |
| OsMIR530 | Rice yield | Sun, W., et al. (2020). “OsmiR530 acts downstream of |
| | | OsPIL15 to regulate grain yield in rice.” New Phytol |
| | | 226(3): 823-837. |
| OsLOGL5 | Rice yield | Wang, C., et al. (2020). “A cytokinin-activation |
| | | enzyme-like gene improves grain yield under various |
| | | field conditions in rice.” Plant Mol Biol 102(4-5): |
| | | 373-388. |
| OsDim1 | Rice yield | Doku, H. A., et al. (2019). “The expression pattern of |
| | | OsDim1 in rice and its proposed function.” Sci Rep |
| | | 9(1): 18492. |
| OsMYC2 | Resistance to bacterial | Uji, Y., et al. (2016). “Overexpression of OsMYC2 |
| | leaf blight in rice | Results in the Up-Regulation of Early JA-Rresponsive |
| | | Genes and Bacterial Blight Resistance in Rice.” Plant |
| | | Cell Physiol 57(9): 1814-1827. |
| Rice NH1 | Resistance to bacterial | Bart, R. S., et al. (2010). “Rice Snl6, a |
| | leaf blight in rice | cinnamoyl-CoA reductase-like gene family member, |
| | | is required for NH1-mediated immunity to |
| | | Xanthomonas oryzae pv. oryzae.” PLoS Genet 6(9): |
| | | e1001123. |
| OsTFX1 | Resistance to bacterial | Sugio, A., et al. (2007). “Two type III effector genes |
| | leaf blight in rice | of Xanthomonas oryzae pv. oryzae control the |
| | | induction of the host genes OsTFIIAgamma1 and |
| | | OsTFX1 during bacterial blight of rice.” Proc Natl |
| | | Acad Sci USA 104(25): 10720-10725. |
| DAO | To improve auxin | Zhao, Z., et al. (2013). A role for a dioxygenase in |
| | catabolism and | auxin metabolism and reproductive development in |
| | maintain auxin | rice. Developmental Cell, 27(1), 113-122. |
| | homeostasis | |
| OsSWEET5 | Growth development | Zhou, Y., et al. (2014). Overexpression of |
| | | OsSWEET5 in rice causes growth retardation and |
| | | precocious senescence. Plos One, 9(4), e94210. |
| OsPHR2 | Growth development | Wu, P., et al.(2008). Role of OsPHR2 on phosphorus |
| | | homeostasis and root hairs development in rice (oryza |
| | | sativa. L.). Plant Signaling & Behavior, 3(9), 674-675. |
| OsPHR1 | Growth development | Wu, P., et al.(2008). Role of OsPHR2 on phosphorus |
| | | homeostasis and root hairs development in rice (oryza |
| | | sativa. L.). Plant Signaling & Behavior, 3(9), 674-675. |
| OsPHF1 | Growth development | Wu, Z., et al. (2011). Investigating the contribution of |
| | | the phosphate transport pathway to arsenic |
| | | accumulation in rice. Plant Physiology, 157(1), |
| | | 498-508. |
| OsMGH3 | Growth development | Vijayraghavan, U.. (2011). Auxin-responsive |
| | | OsMGH3, a common downstream target of |
| | | OsMADS1 and OsMADS6, controls rice floret |
| | | fertility. Plant & Cell Physiology, 52(12), 2123-2135. |
| BZR1 | Growth development | Zhang, L. Y., et al. (2009). Antagonistic HLH/bHLH |
| | | transcription factors mediate brassinosteroid |
| | | regulation of cell elongation and plant development in |
| | | rice and arabidopsis. Plant Cell, 21(12), 3767-3780. |
| OsCPK10 | Biotic and abiotic | Fu, L., et al. (2013). “Overexpression of constitutively |
| | stress (resistance to | active OsCPK10 increases Arabidopsis resistance |
| | Magnaporthe grisea) | against Pseudomonas syringae pv. tomato and rice |
| | | resistance against Magnaporthe grisea.” Plant Physiol |
| | | Biochem 73: 202-210. |
| PME1 | Synthesis of | Kang, K., et al. (2011). “Methanol is an endogenous |
| | tryptophan | elicitor molecule for the synthesis of tryptophan and |
| | | tryptophan-derived secondary metabolites upon |
| | | senescence of detached rice leaves.” Plant J 66(2): |
| | | 247-257. |
| OsMYB5P | Tolerance to | Yang, W. T., et al. (2018). Rice OsMYB5P improves |
| | phosphorous | plant phosphate acquisition by regulation of phosphate |
| | deficiency | transporter. PloS one, 13(3), e0194628. |
| TOND1 | Tolerance to nitrogen | Zhang., et al. (2015).”TOND1 confers tolerance to |
| | deficiency | nitrogen deficiency in rice.”The Plant |
| | | Journal, 81(3): 367-376. |
| OsRZFP34 | To enhance stomata | Hsu, K. H., et al. (2014). “Expression of a gene |
| | opening | encoding a rice RING zinc-finger protein, OsRZFP34, |
| | | enhances stomata opening.” Plant Mol Biol 86(1-2): |
| | | 125-137. |
| P5CS | To increase synthesis | Kaikavoosi, K., et al. (2015). “2-Acetyl-1-pyrroline |
| | of proline and | augmentation in scented indica rice (Oryza sativa L.) |
| | augment rice scent | varieties through Delta(1)-pyrroline-5-carboxylate |
| | | synthetase (P5CS) gene transformation.” Appl |
| | | Biochem Biotechnol 177(7): 1466-1479. |
| OsSultr1; 1 | Tolerance to heavy | Kumar, S., et al. (2019). “Arsenic-responsive |
| | mental | high-affinity rice sulphate transporter, OsSultr1; 1, |
| | | provides abiotic stress tolerance under limiting |
| | | sulphur condition.” J Hazard Mater 373: 753-762. |
| OsCPK4 | Salt and drought | Campo, S., et al. (2014). “Overexpression of a |
| | tolerance | Calcium-Dependent Protein Kinase Confers Salt and |
| | | Drought Tolerance in Rice by Preventing Membrane |
| | | Lipid Peroxidation.” Plant Physiol 165(2): 688-704. |
| oscpk12 | Salt tolerance and | Asano, T., et al. (2012). “A rice calcium-dependent |
| | blast disease | protein kinase OsCPK12 oppositely modulates |
| | resistance | salt-stress tolerance and blast disease resistance.” |
| | | Plant J 69(1): 26-36. |
| serine-threonine | Salt tolerance | Diedhiou, C. J., et al. (2008). “The SNF1-type |
| protein kinase | | serine-threonine protein kinase SAPK4 regulates |
| SAPK4 | | stress-responsive gene expression in rice.” BMC Plant |
| | | Biol 8: 49. |
| Rab16A | Salt tolerance | Ganguly, M., et al. (2012). “Overexpression of |
| | | Rab16A gene in indica rice variety for generating |
| | | enhanced salt tolerance.” Plant Signal Behav 7(4): |
| | | 502-509. |
| STRK1 | Salt stress tolerance | Zhou, Y., et al. (2018). The receptor-like cytoplasmic |
| | | kinase STRK1 phosphorylates and activates Catc, |
| | | thereby regulating H2O2 homeostasis and improving |
| | | salt tolerance in rice. Plant Cell, tpc.01000.2017. |
| OsDREB2A | Salt stress tolerance | Zhang, X. X., et al. (2013). OsDREB2A, a rice |
| | | transcription factor, significantly affects salt tolerance |
| | | in transgenic soybean. Plos One, 8(12), e83011. |
| OsCam1 | Salt stress tolerance | Worawat., et al. (2018). Downstream components of |
| | | the calmodulin signaling pathway in the rice salt |
| | | stress response revealed by transcriptome profiling |
| | | and target identification. BMC Plant Biology. |
| OsiSAP8 | Salt, drought and cold | Kanneganti, V. and A. K. Gupta (2008). |
| | tolerance | “Overexpression of OsiSAP8, a member of stress |
| | | associated protein (SAP) gene family of rice confers |
| | | tolerance to salt, drought and cold stress in transgenic |
| | | tobacco and rice.” Plant Mol Biol 66(5): 445-462. |
| OsTZF1 | Salt and drought | Jan, A., et al. (2013). “OsTZF1, a CCCH-tandem zinc |
| | tolerance | finger protein, confers delayed senescence and stress |
| | | tolerance in rice by regulating stress-related genes.” |
| | | Plant Physiol 161(3): 1202-1216. |
| OsLEA5 | Salt and drought | Huang, L., et al. (2018). “An Atypical Late |
| | tolerance | Embryogenesis Abundant Protein OsLEA5 Plays a |
| | | Positive Role in ABA-Induced Antioxidant Defense in |
| | | Oryza sativa L.” Plant Cell Physiol 59(5): 916-929. |
| ZFP182 | Salt tolerance | Huang, J., et al (2007). A novel rice C2H2-type zinc |
| | | finger protein lacking DLN-box/EAR-motif plays a |
| | | role in salt tolerance. Biochimica et Biophysica Acta |
| | | (BBA)-Gene Structure and Expression, 1769(4), |
| | | 220-227. |
| OsSta2 | Salt tolerance | Kumar, M., et al. (2017). “Ectopic Expression of |
| | | OsSta2 Enhances Salt Stress Tolerance in Rice.” Front |
| | | Plant Sci 8: 316. |
| OsMSRA4.1 | Salt tolerance | Guo, X., et al. (2009). “OsMSRA4.1 and OsMSRB1.1, |
| | | two rice plastidial methionine sulfoxide reductases, |
| | | are involved in abiotic stress responses.” Planta |
| | | 230(1): 227-238. |
| OsMPG1 | Salt tolerance | Kumar, R., et al. (2012). “Functional screening of |
| | | cDNA library from a salt tolerant rice genotype |
| | | Pokkali identifies mannose-1-phosphate guanyl |
| | | transferase gene (OsMPG1) as a key member of |
| | | salinity stress response.” Plant Mol Biol 79(6): |
| | | 555-568. |
| OsMKK6 | Salt tolerance | Kumar, K. and A. K. Sinha (2013). “Overexpression |
| | | of constitutively active mitogen activated protein |
| | | kinase kinase 6 enhances tolerance to salt stress in |
| | | rice.” Rice (NY) 6(1): 25. |
| OsHAK1 | Salt tolerance | Chen, G., et al. (2015). “Rice potassium transporter |
| | | OsHAK1 is essential for maintaining |
| | | potassium-mediated growth and functions in salt |
| | | tolerance over low and high potassium concentration |
| | | ranges.” Plant Cell Environ 38(12): 2747-2765. |
| OsEXPA7 | Salt tolerance | Jadamba, C., et al. (2020). “Overexpression of Rice |
| | | Expansin7 (Osexpa7) Confers Enhanced Tolerance to |
| | | Salt Stress in Rice.” Int J Mol Sci 21(2). |
| MIPS | Salt tolerance | Kusuda, H., et al. (2015). “Ectopic expression of |
| | | myo-inositol 3-phosphate synthase induces a wide |
| | | range of metabolic changes and confers salt tolerance |
| | | in rice.” Plant Sci 232: 49-56. |
| CYP94C2b | Salt tolerance | Kurotani, K., et al. (2015). “Stress Tolerance Profiling |
| | | of a Collection of Extant Salt-Tolerant Rice Varieties |
| | | and Transgenic Plants Overexpressing Abiotic Stress |
| | | Tolerance Genes.” Plant Cell Physiol 56(10): |
| | | 1867-1876. |
| CYP94C2b | Salt tolerance | Kurotani, K., et al. (2015). “Elevated levels of CYP94 |
| | | family gene expression alleviate the jasmonate |
| | | response and enhance salt tolerance in rice.” Plant |
| | | Cell Physiol 56(4): 779-789. |
| SUB1A | Flooding tolerance | Fukao, T., et al. (2011). “The submergence tolerance |
| | | regulator SUB1A mediates crosstalk between |
| | | submergence and drought tolerance in rice.” Plant Cell |
| | | 23(1): 412-427. |
| OsCBL10 | Flooding tolerance | Ye, N. H., et al. (2018). Natural variation in the |
| | | promoter of rice calcineurin b-like protein10 |
| | | (OsCBL10) affects flooding tolerance during seed |
| | | germination among rice subspecies. Plant Journal for |
| | | Cell & Molecular Biology. |
| SNAC2 | Stress tolerance | Hu, H., et al. (2008). “Characterization of |
| | | transcription factor gene SNAC2 conferring cold and |
| | | salt tolerance in rice.” Plant Mol Biol 67(1-2): |
| | | 169-181. |
| OsNCED5 | Stress tolerance | Huang, Y., et al. (2019). “OsNCED5, a |
| | | 9-cis-epoxycarotenoid dioxygenase gene, regulates |
| | | salt and water stress tolerance and leaf senescence in |
| | | rice.” Plant Sci 287: 110188. |
| OsLea14-A | Stress tolerance | Hu, T., et al. (2019). “Overexpression of OsLea14-A |
| | | improves the tolerance of rice and increases Hg |
| | | accumulation under diverse stresses.” Environ Sci |
| | | Pollut Res Int 26(11): 10537-10551. |
| OsCYP20-2 | Stress tolerance | Kim, S. K., et al. (2012). “The rice thylakoid lumenal |
| | | cyclophilin OsCYP20-2 confers enhanced |
| | | environmental stress tolerance in tobacco and |
| | | Arabidopsis.” Plant Cell Rep 31(2): 417-426. |
| OsGLYII-2 | Salt tolerance | Ghosh, A., et al. (2014). “A glutathione responsive |
| | | rice glyoxalase II, OsGLYII-2, functions in salinity |
| | | adaptation by maintaining better photosynthesis |
| | | efficiency and anti-oxidant pool.” Plant J 80(1): |
| | | 93-105. |
| OsDREB1B | Biotic and abiotic | Gutha, L. R. and A. R. Reddy (2008). “Rice DREB1B |
| | stress tolerance | promoter shows distinct stress-specific responses, and |
| | | the overexpression of cDNA in tobacco confers |
| | | improved abiotic and biotic stress tolerance.” Plant |
| | | Mol Biol 68(6): 533-555. |
| OsPRX38 | Arsenic tolerance | Kidwai, M., et al. (2019). “Oryza sativa class III |
| | | peroxidase (OsPRX38) overexpression in Arabidopsis |
| | | thaliana reduces arsenic accumulation due to |
| | | apoplastic lignification.” J Hazard Mater 362: |
| | | 383-393. |
| OsAIR2 | Arsenic tolerance | Hwang, S. G., et al. (2017). “Molecular |
| | | characterization of rice arsenic-induced RING finger |
| | | E3 ligase 2 (OsAIR2) and its heterogeneous |
| | | overexpression in Arabidopsis thaliana.” Physiol Plant |
| | | 161(3): 372-384. |
| OsAIR1 | Arsenic tolerance | Hwang, S. G., et al. (2016). “Molecular |
| | | characterization of Oryza sativa arsenic-induced |
| | | RING E3 ligase 1 (OsAIR1): Expression patterns, |
| | | localization, functional interaction, and heterogeneous |
| | | overexpression.” J Plant Physiol 191: 140-148. |
| OsRGB1 | Thermo and salt | Biswas, S., et al. (2019). “Overexpression of |
| | tolerance | heterotrimeric G protein beta subunit gene (OsRGB1) |
| | | confers both heat and salinity stress tolerance in rice.” |
| | | Plant Physiol Biochem 144: 334-344. |
| OsPTF1 | Low Phosphate | Keke Yi., et al. OsPTF1, a Novel Transcription Factor |
| | tolerance | Involved inTolerance to Phosphate Starvation in Rice |
| OsSHMT, | Chilling tolerance | Fang, C., et al. (2020). “Serine |
| Lsi1 gene | | hydroxymethyltransferase localised in the |
| (Lsi1-OX) | | endoplasmic reticulum plays a role in scavenging |
| | | H2O2 to enhance rice chilling tolerance.” BMC Plant |
| | | Biol 20(1): 236. |
| OsICE | Chilling tolerance | Deng, C., et al. (2017). “The rice transcription factors |
| | | OsICE confer enhanced cold tolerance in transgenic |
| | | Arabidopsis.” Plant Signal Behav 12(5): e1316442. |
| ZFP252 | Drought and salt | Dong-Qing Xu., et al (2008). Overexpression of a |
| | tolerance | TFIIIA-type zinc finger protein geneZFP252 enhances |
| | | drought and salt tolerance in rice (Oryza sativa L.) |
| OsETOL1 | Drought and flooding | Du, H., et al. (2014). “A homolog of ETHYLENE |
| | tolerance | OVERPRODUCER, OsETOL1, differentially |
| | | modulates drought and submergence tolerance in |
| | | rice.” Plant J 78(5): 834-849. |
| OsRab7 | To improve drought | El-Esawi, M. A. and A. A. Alayafi (2019). |
| | and heat tolerance and | “Overexpression of Rice Rab7 Gene Improves |
| | increase rice yield | Drought and Heat Tolerance and Increases Grain |
| | | Yield in Rice (Oryza sativa L.).” Genes (Basel) 10(1). |
| OsGH3-2 | Drought and cold | Du, H., et al. (2012). “A GH3 family member, |
| | tolerance | OsGH3-2, modulates auxin and abscisic acid levels |
| | | and differentially affects drought and cold tolerance in |
| | | rice.” J Exp Bot 63(18): 6467-6480. |
| OsbZIP23 | Drought tolerance and | Dey, A., et al. (2016). “Enhanced Gene Expression |
| | yield increase | Rather than Natural Polymorphism in Coding |
| | | Sequence of the OsbZIP23 Determines Drought |
| | | Tolerance and Yield Improvement in Rice |
| | | Genotypes.” PLoS One 11(3): e0150763. |
| ScMYBAS1 | Drought tolerance | Favero Peixoto-Junior, R., et al. (2018). |
| | | “Overexpression of ScMYBAS1 alternative splicing |
| | | transcripts differentially impacts biomass |
| | | accumulation and drought tolerance in rice transgenic |
| | | plants.” PLoS One 13(12): e0207534. |
| OsTF1L | Drought tolerance | Bang, S. W., et al. (2019). “Overexpression of |
| | | OsTFIL, a rice HD-Zip transcription factor, promotes |
| | | lignin biosynthesis and stomatai closure that improves |
| | | drought tolerance.” Plant Biotechnol J 17(1): 118-131. |
| OsPLDα1 | Drought tolerance | Abreu, F. R. M., et al. (2018). “Overexpression of a |
| | | phospholipase (OsPLDalpha1) for drought tolerance |
| | | in upland rice (Oryza sativa L.).” Protoplasma 255(6): |
| | | 1751-1761. |
| OsDIL | Drought tolerance | Guo, C., et al. (2013). “The rice OsDIL gene plays a |
| | | role in drought tolerance at vegetative and |
| | | reproductive stages.” Plant Mol Biol 82(3): 239-253. |
| DST | Drought and salt | Cui, L. G., et al. (2015). “DCA1 Acts as a |
| &DCA1 | tolerance | Transcriptional Co-activator of DST and Contributes |
| | | to Drought and Salt Tolerance in Rice.” PLoS Genet |
| | | 11(10): e1005617. |
| OsAHL1 | Drought tolerance | Zhou, L., et al (2016). “A novel gene OsAHL1 |
| | | improvesboth drought avoidance anddrought tolerance |
| | | in rice.” Scientific reports, 6(1), 1-15. |
| OsNAC5 | Drought tolerance and | Jeong, J. S., et al. (2013). “OsNAC5 overexpression |
| | yield increase | enlarges root diameter in rice plants leading to |
| | | enhanced drought tolerance and increased grain yield |
| | | in the field.” Plant Biotechnol J 11(1): 101-114. |
| OsMAPK5 | Drought, salt, and cold | Xiong, L., et al.(2003). “Disease Resistance and |
| | tolerance; disease | Abiotic Stress Tolerance in Rice Are Inversely |
| | resistance | Modulated by an Abscisic Acid-Inducible |
| | | Mitogen-Activated Protein Kinase.” Plant Cell. |
| OsNAC10 | Drought, salt and cold | Jeong, J. S., et al. (2010). “Root-specific expression of |
| | tolerance | OsNAC10 improves drought tolerance and grain yield |
| | | in rice under field drought conditions.” Plant Physiol |
| | | 153(1): 185-197. |
| ONAC022 | Drought and salt | Hong, Y., et al. (2016). “Overexpression of a |
| | tolerance | Stress-Responsive NAC Transcription Factor Gene |
| | | ONAC022 Improves Drought and Salt Tolerance in |
| | | Rice.” Front Plant Sci 7: 4. |
| ZFP245 | Tolerance to drought, | Huang, J., et al. (2009). “Increased tolerance of rice to |
| | cold and oxidative | cold, drought and oxidative stresses mediated by the |
| | stresses | overexpression of a gene that encodes the zinc finger |
| | | protein ZFP245.” Biochem Biophys Res Commun |
| | | 389(3): 556-561. |
| OsASR1 | Drought and cold | Joo, J., et al. (2013). “Abiotic stress responsive rice |
| | tolerance | ASR1 and ASR3 exhibit different tissue-dependent |
| | | sugar and hormone-sensitivities.” Mol Cells 35(5): |
| | | 421-435. |
| AP37 | Drought, high salt and | Kim, Y. S. and J. K. Kim (2009). “Rice transcription |
| | cold tolerance | factor AP37 involved in grain yield increase under |
| | | drought stress.” Plant Signal Behav 4(8): 735-736. |
| OSRIP18 | Drought and heat | Jiang, S. Y., et al. (2012). “Over-expression of |
| | tolerance | OSRIP18 increases drought and salt tolerance in |
| | | transgenic rice plants.” Transgenic Res 21(4): |
| | | 785-795. |
| WRKY13 | Drought tolerance | Xiao, J., et al.(2013). “Rice WRKY13 Regulates Cross |
| | | Talk between Abiotic and Biotic Stress Signaling |
| | | Pathways by Selective Binding to Different |
| | | cis-Elements.” Plant Physiology, 163(4), 1868-1882. |
| SNAC1 | Drought tolerance | Hu, H., et al. (2006). “Overexpressing a NAM, ATAF, |
| | | and CUC (NAC) transcription factor enhances drought |
| | | resistance and salt tolerance in rice.” Proc Natl Acad |
| | | Sci USA 103(35): 12987-12992. |
| OsRBGD3 | Drought tolerance | Lenka, S. K., et al. (2019). “Heterologous expression |
| | | of rice RNA-binding glycine-rich (RBG) gene |
| | | OsRBGD3 in transgenic Arabidopsis thaliana confers |
| | | cold stress tolerance.” Funct Plant Biol 46(5): |
| | | 482-491. |
| OsPYL6 | Drought tolerance | Kumar, V. V. S., et al. (2020). “ABA receptor |
| | | OsPYL6 confers drought tolerance to indica rice |
| | | through dehydration avoidance and tolerance |
| | | mechanisms.” J Exp Bot. |
| OsPYL3 | Drought tolerance | Lenka, S. K., et al. (2018). “Ectopic Expression of |
| | | Rice PYL3 Enhances Cold and Drought Tolerance in |
| | | Arabidopsis thaliana.” Mol Biotechnol 60(5): |
| | | 350-361. |
| OsNF-YA7 | Drought tolerance | Lee, D. K., et al. (2015). “The NF-YA transcription |
| | | factor OsNF-YA7 confers drought stress tolerance of |
| | | rice in an abscisic acid independent manner.” Plant |
| | | Sci 241: 199-210. |
| OsNADK1 | Drought tolerance | Wang, X., et al. (2020). “The NAD kinase OsNADK1 |
| | | affects theintracellular redox balance and enhances |
| | | the tolerance of rice to drought.” BMC Plant Biology, |
| | | 20. |
| OsNAC6 | Drought tolerance | Lee, D. K., et al. (2017). “The rice OsNAC6 |
| | | transcription factor orchestrates multiple molecular |
| | | mechanisms involving root structural adaptions and |
| | | nicotianamine biosynthesis for drought tolerance.” |
| | | Plant Biotechnol J 15(6): 754-764. |
| OsIAA6 | Drought tolerance | Jung, H., et al. (2015). “OsIAA6, a member of the rice |
| | | Aux/IAA gene family, is involved in drought |
| | | tolerance and tiller outgrowth.” Plant Sci 236: |
| | | 304-312. |
| OsERF71 | Drought tolerance | Lee, D. K., et al. (2017). “Rice OsERF71-mediated |
| | | root modification affects shoot drought tolerance.” |
| | | Plant Signal Behav 12(1): e1268311. |
| OsDRAP1 | Drought tolerance | Huang, L., et al. (2018). “Characterization of |
| | | Transcription Factor Gene OsDRAP1 Conferring |
| | | Drought Tolerance in Rice.” Front Plant Sci 9: 94. |
| OsCYP18-2 | Drought tolerance | Lee, S. S., et al. (2015). “Rice cyclophilin OsCYP18-2 |
| | | is translocated to the nucleus by an interaction with |
| | | SKIP and enhances drought tolerance in rice and |
| | | Arabidopsis.” Plant Cell Environ 38(10): 2071-2087. |
| OsCPK9 | Drought tolerance | Shuya, Wei., et al.(2014). “A rice calcium-dependent |
| | | protein kinase OsCPK9positively regulates drought |
| | | stress tolerance andspikelet fertility.” BMC Plant |
| | | Biology, 14(1), 133-133. |
| OsADF | Drought tolerance | Huang, Y. C., et al. (2012). “Comprehensive analysis |
| | | of differentially expressed rice actin depolymerizing |
| | | factor gene family and heterologous overexpression of |
| | | OsADF3 confers Arabidopsis Thaliana drought |
| | | tolerance.” Rice (NY) 5(1): 33. |
| OCPI1 | Drought tolerance | Huang, Y., et al. (2007). “Characterization of a stress |
| | | responsive proteinase inhibitor gene with positive |
| | | effect in improving drought resistance in rice.” Planta |
| | | 226(1): 73-85. |
| CDPK13 | Drought tolerance | Komatsu, S., et al. (2007). “Over-expression of |
| | | calcium-dependent protein kinase 13 and calreticulin |
| | | interacting protein 1 confers cold tolerance on rice |
| | | plants.” Mol Genet Genomics 277(6): 713-723. |
| OsDREB1D | Cold and salt tolerance | Zhang, Y., et al. (2009). “Expression of a rice DREB1 |
| | | gene, OsDREB1D, enhances cold and high-salt |
| | | tolerance in transgenic Arabidopsis.” Bmb Reports, |
| | | 42(8), 486-492. |
| OsWRKY71 | Cold tolerance | Kumar, M., et al. (2017). “Genome-Wide |
| | | Identification and Analysis of Genes, Conserved |
| | | between japonica and indica Rice Cultivars, that |
| | | Respond to Low-Temperature Stress at the Vegetative |
| | | Growth Stage.” Front Plant Sci 8: 1120. |
| OsLti6b | Cold tolerance | Kim, S. H., et al. (2007). “Isolation of cold |
| | | stress-responsive genes in the reproductive organs, |
| | | and characterization of the OsLti6b gene from rice |
| | | (Oryza sativa L.).” Plant Cell Rep 26(7): 1097-1110. |
| OsAOX1a | Cold tolerance | Li, C. R., et al. (2013). “Overexpression of an |
| | | alternative oxidase gene, OsAOX1a, improves cold |
| | | tolerance in Oryza sativa L.” Genet Mol Res 12(4): |
| | | 5424-5432. |
| OsREX1-S | Cadmium tolerance | Kunihiro, S., et al. (2014). “Overexpression of rice |
| | | OsREX1-S, encoding a putative component of the |
| | | core general transcription and DNA repair factor IIH, |
| | | renders plant cells tolerant to cadmium- and |
| | | UV-induced damage by enhancing DNA excision |
| | | repair.” Planta 239(5): 1101-1111. |
| OsMYB45 | Cadmium tolerance | Hu, S., et al. (2017). “OsMYB45 plays an important |
| | | role in rice resistance to cadmium stress.” Plant Sci |
| | | 264: 1-8. |
| OsSIRP1 | Tolerance to salinity | Hwang, S. G., et al. (2016). “Molecular dissection of |
| | and other stresses | Oryza sativa salt-induced RING Finger Protein 1 |
| | | (OsSIRP1): possible involvement in the sensitivity |
| | | response to salinity stress.” Physiol Plant 158(2): |
| | | 168-179 |
| OsJRL | Tolerance to salinity | He, X., et al. (2017). “A rice jacalin-related |
| | and other stresses | mannose-binding lectin gene, OsJRL, enhances |
| | | Escherichia coli viability under high salinity stress |
| | | and improves salinity tolerance of rice.” Plant Biol |
| | | (Stuttg) 19(2): 257-267. |
| SLG1 | Heat tolerance | Xu, Y., et al.(2020). “Natural variations of SLG1 |
| | | confer high-temperaturetolerance in indica |
| | | rice.”Nature Communications, 11(1), 5441. |
| SBPase | Heat tolerance | Feng, L., et al. (2007). “Overexpression of SBPase |
| | | enhances photosynthesis against high temperature |
| | | stress in transgenic rice plants.” Plant Cell Rep 26(9): |
| | | 1635-1646. |
| OsCDPK1 | Drought tolerance | Ho, S. L., et al. (2013). “Sugar starvation- and |
| | | GA-inducible calcium-dependent protein kinase 1 |
| | | feedback regulates GA biosynthesis and activates a |
| | | 14-3-3 protein to confer drought tolerance in rice |
| | | seedlings.” Plant Mol Biol 81(4-5): 347-361. |
| OsASR5 | Drought tolerance | Li, J., et al. (2017).”OsASR5 enhances drought |
| | | tolerance through a stomatai closure pathway |
| | | associated with ABA and H2O2 signalling in |
| | | rice.”Plant Biotechnology Journal.. |
| OsZFP350 | Abiotic stress | Kang, Z., et al. (2019). “Overexpression of the zinc |
| | tolerance | finger protein gene OsZFP350 improves root |
| | | development by increasing resistance to abiotic stress |
| | | in rice.” Acta Biochim Pol 66(2): 183-190. |
| OsTOP6A1 | Abiotic stress | Jain, M., et al. (2008). “Constitutive expression of a |
| | tolerance | meiotic recombination protein gene homolog, |
| | | OsTOP6A1, from rice confers abiotic stress tolerance |
| | | in transgenic Arabidopsis plants.” Plant Cell Rep |
| | | 27(4): 767-778. |
| OsGSTL2 | Abiotic stress | Kumar, S., et al. (2013). “Expression of a rice Lambda |
| | tolerance | class of glutathione S-transferase, OsGSTL2, in |
| | | Arabidopsis provides tolerance to heavy metal and |
| | | other abiotic stresses.” J Hazard Mater 248-249: |
| | | 228-237. |
| OsCyp2-P | Abiotic stress | Kumari, S., et al. (2015). “Expression of a cyclophilin |
| | tolerance | OsCyp2-P isolated from a salt-tolerant landrace of |
| | | rice in tobacco alleviates stress via ion homeostasis |
| | | and limiting ROS accumulation.” Funct Integr |
| | | Genomics 15(4): 395-412. |
| ZFP177 | To enhance tolerance | Huang, J., et al. (2008). “Expression analysis of rice |
| | to high and low | A20/AN1-type zinc finger genes and characterization |
| | temperatures and | of ZFP177 that contributes to temperature stress |
| | sensitivity to salt and | tolerance.” Gene 420(2): 135-144. |
| | drought | |
| OsCAF1B | Low-temperature | Fang, J. C., et al. (2021). “A CCR4-associated factor |
| | tolerance | 1, OsCAF1B, confers tolerance of low-temperature |
| | | stress to rice seedlings.” Plant Mol Biol 105(1-2): |
| | | 177-192. |
| OsPEX1 | Lignin content | Ke, S., et al. (2019). “Rice OsPEX1, an extensin-like |
| | | protein, affects lignin biosynthesis and plant growth.” |
| | | Plant Mol Biol 100(1-2): 151-161. |
| OsLRR1 | Immune response | Liang, Z., et al. (2010). “A novel simple extracellular |
| | | leucine-rich repeat (eLRR) domain protein from rice |
| | | (OsLRR1) enters the endosomal pathway and interacts |
| | | with the hypersensitive-induced reaction protein 1 |
| | | (OsHIRl).” Plant Cell & Environment, 32. |
| OsHIR1 | Immune response | Liang, Z., et al. (2010). “A novel simple extracellular |
| | | leucine-rich repeat (eLRR) domain protein from rice |
| | | (OsLRR1) enters the endosomal pathway and interacts |
| | | with the hypersensitive-induced reaction protein 1 |
| | | (OsHIR1).” Plant Cell & Environment, 32. |
| OsPIN2 | Aluminium tolerance | D Wu., et al.(2015). “Overexpressing OsPIN2 |
| | | enhances aluminium internalization by elevating |
| | | vesicular trafficking in rice root apex.” Journal of |
| | | Experimental Botany, 66(21), 6791-6801. |
| PGL2 | Grain length and | Heang, D. and H. Sassa (2012). “An atypical bHLH |
| | weight | protein encoded by POSITIVE REGULATOR OF |
| | | GRAIN LENGTH 2 is involved in controlling grain |
| | | length and weight of rice through interaction with a |
| | | typical bHLH protein APG.” Breed Sci 62(2): |
| | | 133-141. |
| PGL1 | Grain length and | Heang, D. and H. Sassa (2012). “Antagonistic actions |
| | weight | of HLH/bHLH proteins are involved in grain length |
| | | and weight in rice.” PLoS One 7(2): e31325. |
| OsCKX2 | Grain number and | Yang, J., et al. (2018). “Chromatin interacting factor |
| | yield | OsVIL2 increases biomass and rice grain yield.” Plant |
| | | Biotechnology Journal. |
| OsGL1-2 | Wax accumulation and | Islam, M. A., et al. (2009). “Characterization of |
| | drought resistance | Glossy1-homologous genes in rice involved in leaf |
| | | wax accumulation and drought resistance.” Plant Mol |
| | | Biol 70(4): 443-456. |
| GLA1 | To regulate grain size | Wang, T., et al. (2019). “GRAIN LENGTH AND |
| | | AWN 1 negatively regulates grain size in rice.” J |
| | | Integr Plant Biol 61(10): 1036-1042. |
| OsIQD14 | To regulate grain | Yang, B. J., et al. “Rice microtubule-associated |
| | shape | protein IQ67-DOMAIN14 regulates grain shape by |
| | | modulating microtubule cytoskeleton |
| | | dynamics.” Wiley-Blackwell Online Open, 18(5). |
| OsAPx2 | Salt tolerance | Guan, Q., et al. (2012). “Genetic transformation and |
| | | analysis of rice OsAPx2 gene in Medicago sativa.” |
| | | PLoS One 7(7): e41233. |
| TPS46 | Aphid resistance | Sun, Y., et al. (2017). “TPS46, a Rice Terpene |
| | | Synthase Conferring Natural Resistance to Bird |
| | | Cherry-Oat Aphid, Rhopalosiphum padi (Linnaeus).” |
| | | Front Plant Sci 8: 110. |
| OsNCED3 | Stress resistance | Huang, Y., et al. (2018). “9-cis-Epoxycarotenoid |
| | | Dioxygenase 3 Regulates Plant Growth and Enhances |
| | | Multi-Abiotic Stress Tolerance in Rice.” Front Plant |
| | | Sci 9: 162. |
| OsNPR1 | Rice bacterial leaf | Yuexing Yuan., et al.(2010). “Functional analysis of |
| | blight resistance | rice NPR1-like genes reveals that OsNPR1/NH1 is the |
| | | rice orthologue conferring disease resistance with |
| | | enhance herbivore susceptibility.” Plant Biotechnology |
| | | Journal, 5(2), 313-324. |
| OsOXO4 | Sheath blight | Kutubuddin A Molla., et al.(2013). “Rice oxalate |
| | resistance | oxidase gene driven by green tissue-specific promoter |
| | | increases tolerance to sheath blight pathogen |
| | | (Rhizoctonia solani) in transgenic rice. “Molecular |
| | | Plant Pathology, 14(9). |
| RDR6 | Rice stripe disease | Hong, W., et al. (2015). “OsRDR6 plays role in host |
| | resistance | defense against double-stranded RNA virus, Rice |
| | | Dwarf Phytoreovirus.” Sci Rep 5: 11324. |
| OsHsp18.0 | Biotic and abiotic | Kuang, J., et al. (2017). “A Class II small heat shock |
| | stress tolerance | protein OsHsp18.0 plays positive roles in both biotic |
| | | and abiotic defense responses in rice.” Sci Rep 7(1): |
| | | 11333. |
| OsTPP1 | Salt stress, cold stress | Ge, L. F., et al. (2008). “Overexpression of the |
| | tolerance | trehalose-6-phosphate phosphatase gene OsTPP1 |
| | | confers stress tolerance in rice and results in the |
| | | activation of stress responsive genes.” Planta 228(1): |
| | | 191-201. |
| ZFP185 | Stress resistance | Ye, Zhang., et al.(2016). “An A20/AN1-type zinc |
| | | finger protein modulates gibberellins and abscisic acid |
| | | contents and increases sensitivity to abiotic stress in |
| | | rice (Oryza sativa).” Journal of Experimental Botany. |
| M2H | Stress resistance | Choi, G. H. and K. Back (2019). “Suppression of |
| | | Melatonin 2-Hydroxylase Increases Melatonin |
| | | Production Leading to the Enhanced Abiotic Stress |
| | | Tolerance against Cadmium, Senescence, Salt, and |
| | | Tunicamycin in Rice Plants.” Biomolecules 9(10). |
| OsSAP1 | Stress resistance | Kothari, K. S., et al. (2016). “Rice Stress Associated |
| | | Protein 1 (OsSAP1) Interacts with Aminotransferase |
| | | (OsAMTR1) and Pathogenesis-Related 1a Protein |
| | | (OsSCP) and Regulates Abiotic Stress Responses.” |
| | | Front Plant Sci 7: 1057. |
| OsNAS2 | Stress tolerance | Lee, S., et al. (2012). “Activation of Rice |
| | | nicotianamine synthase 2 (OsNAS2) enhances iron |
| | | availability for biofortification.” Mol Cells 33(3): |
| | | 269-275. |
| OsNAC9 | Drought tolerance and | Redillas, M. C., et al. (2012). “The overexpression of |
| | crop yield | OsNAC9 alters the root architecture of rice plants |
| | | enhancing drought resistance and grain yield under |
| | | field conditions.” Plant Biotechnol J 10(7): 792-805. |
| OsTPKb | Drought tolerance | Ahmad, I., et al. (2016). “Overexpression of the |
| | | potassium channel TPKb in small vacuoles confers |
| | | osmotic and drought tolerance to rice.” New Phytol |
| | | 209(3): 1040-1048. |
| OsFTL10 | Drought tolerance | Fang, M., et al. (2019). “Overexpression of OsFTL10 |
| | | induces early flowering and improves drought |
| | | tolerance in Oryza sativa L.” PeerJ 7: e6422. |
| OsbZIP16 | Drought tolerance | Chen, H., et al. (2012). “Basic leucine zipper |
| | | transcription factor OsbZIP16 positively regulates |
| | | drought resistance in rice.” Plant Sci 193-194: 8-17. |
| OsAKT1 | Drought tolerance | Ahmad, I., et al. (2016). “Overexpression of the rice |
| | | AKT1 potassium channel affects potassium nutrition |
| | | and rice drought tolerance.” J Exp Bot 67(9): |
| | | 2689-2698. |
| OsITPK2 | Drought and salt | Du, H., et al. (2011). “Characterization of an inositol |
| | tolerance | 1,3,4-trisphosphate 5/6-kinase gene that is essential |
| | | for drought and salt stress responses in rice.” Plant |
| | | Mol Biol 77(6): 547-563. |
| OsSRO1c | Drought tolerance and | You, J., et al.(2013) “The SNAC1-targeted gene |
| | oxidation resistance | OsSRO1c modulates stomatai closure and oxidative |
| | | stress tolerance by regulating hydrogen peroxide in |
| | | rice.”Journal of Experimental Botany(2), 569. |
| OsbZIP46CA1&SAPK6 | Drought and | Chang, Y., et al. (2017). “Co-overexpression of the |
| | temperature tolerance | Constitutively Active Form of OsbZIP46 and |
| | | ABA-Activated Protein Kinase SAPK6 Improves |
| | | Drought and Temperature Stress Resistance in Rice.” |
| | | Front Plant Sci 8: 1102. |
| DSM2/OsBCH1 | Drought tolerance and | Du, H., et al. (2010). “Characterization of the |
| | oxidation resistance | beta-carotene hydroxylase gene DSM2 conferring |
| | | drought and oxidative stress resistance by increasing |
| | | xanthophylls and abscisic acid synthesis in rice.” Plant |
| | | Physiol 154(3): 1304-1318. |
| ZBED | Drought tolerance and | Zuluaga, A. P., et al. (2020). “The Rice DNA-Binding |
| | disease resistance | Protein ZBED Controls Stress Regulators and |
| | | Maintains Disease Resistance After a Mild Drought.” |
| | | Front Plant Sci 11: 1265. |
| ROC4 | Drought tolerance | Wang, Z., et al. (2018).”The E3 Ligase DROUGHT |
| | | HYPERSENSITIVE Negatively Regulates Cuticular |
| | | Wax Biosynthesis by Promoting the Degradation of |
| | | Transcription Factor ROC4 in Rice.” Plant Cell, |
| | | tpc.00823.2017. |
| OsPP18 | Drought tolerance | You, J., et al. (2014). “A STRESS-RESPONSIVE |
| | | NAC1-Regulated Protein Phosphatase Gene Rice |
| | | Protein Phosphatase18 Modulates Drought and |
| | | Oxidative Stress Tolerance through Abscisic |
| | | Acid-Independent Reactive Oxygen Species |
| | | Scavenging in Rice. “Plant Physiology, 166(4), |
| | | 2100-14. |
| OsERF71 | Drought tolerance | Lee, D. K., et al. (2016). “Overexpression of the |
| | | OsERF71 Transcription Factor Alters Rice Root |
| | | Structure and Drought Resistance.” Plant Physiol |
| | | 172(1): 575-588. |
| OsDERF1 | Drought tolerance | Wan, L., et al. (2011). “Transcriptional activation of |
| | | OsDERF1 in OsERF3 and OsAP2-39 negatively |
| | | modulates ethylene synthesis and drought tolerance in |
| | | rice.” PLoS One 6(9): e25216. |
| LRK2 | Drought tolerance | Kang, J., et al. (2017). “Overexpression of the |
| | | leucine-rich receptor-like kinase gene LRK2 increases |
| | | drought tolerance and tiller number in rice.” Plant |
| | | Biotechnol J 15(9): 1175-1185. |
| DHS | Drought tolerance | “The E3 Ligase DROUGHT HYPERSENSITIVE |
| | | Negatively Regulates Cuticular Wax Biosynthesis by |
| | | Promoting the Degradation of Transcription Factor |
| | | ROC4 in Rice” |
| OsCTZFP8 | Cold resistance | Jin, Y. M., et al. (2018). “Overexpression of a New |
| | | Zinc Finger Protein Transcription Factor OsCTZFP8 |
| | | Improves Cold Tolerance in Rice.” Int J Genomics |
| | | 2018: 5480617. |
| OsPYL/RCAR5 | Abiotic stress | Kim, H., et al. (2014). “Overexpression of PYL5 in |
| | tolerance | rice enhances drought tolerance, inhibits growth, and |
| | | modulates gene expression.” J Exp Bot 65(2): |
| | | 453-464. |
| OsF3H | Plant hopper | Jan, R., et al. (2020). “Overexpression of OsF3H |
| | resistance | modulates WBPH stress by alteration of |
| | | phenylpropanoid pathway at a transcriptomic and |
| | | metabolomic level in Oryza sativa.” Sci Rep 10(1): |
| | | 14685. |
| OsACS2 | Magnaporthe oryzae | Helliwell, E. E., et al. (2013). “Transgenic rice with |
| | and Rhizoctonia solani | inducible ethylene production exhibits broad-spectrum |
| | disease resistance | disease resistance to the fungal pathogens |
| | | Magnaporthe oryzae and Rhizoctonia solani.” Plant |
| | | Biotechnol J 11(1): 33-42. |
| OsWRKY76 | Magnaporthe oryzae | Naoki, Y., et al. (2013). “WRKY76 is a rice |
| | resistance and drought | transcriptional repressor playing opposite roles in |
| | tolerance | blast disease resistance and cold stress |
| | | tolerance.” Journal of Experimental Botany(16), |
| | | 5085-5097. |
| OsEREB1 | Magnaporthe oryzae | Jisha, V., et al. (2015). “Overexpression of an |
| | and xanthomonas | AP2/ERF Type Transcription Factor OsEREBP1 |
| | oryzae resistance; | Confers Biotic and Abiotic Stress Tolerance in Rice.” |
| | drought tolerance | PLoS One 10(6): e0127831. |
| OsWRKY31 | Magnaporthe oryzae | Juan., et al.(2008) “Constitutive expression of |
| | resistance | pathogen-inducible OsWRKY31 enhances disease |
| | | resistance and affects root growth and auxin response |
| | | in transgenic rice plants.” Cell Research. |
| OsMBL1 | Magnaporthe oryzae | Han, Y., et al. (2019). “A Magnaporthe Chitinase |
| | resistance | Interacts with a Rice Jacalin-Related Lectin to |
| | | Promote Host Colonization.” Plant Physiol 179(4): |
| | | 1416-1430. |
| CYP71Z18 | Magnaporthe oryzae | Shen, Q., et al. (2019). “CYP71Z18 overexpression |
| | resistance | confers elevated blast resistance in transgenic rice.” |
| | | Plant Mol Biol 100(6): 579-589. |
| OsEXTL | Plant lodging | Fan, C., et al. (2018). “Ectopic expression of a novel |
| | resistance | OsExtensin-like gene consistently enhances plant |
| | | lodging resistance by regulating cell elongation and |
| | | cell wall thickening in rice.” Plant Biotechnol J 16(1): |
| | | 254-263. |
| S1R109944 | Disease resistance | Qiao, L., et al. (2020). “Expression of rice siR109944 |
| | | in Arabidopsis affects plant immunity to multiple |
| | | fungal pathogens.” Plant Signal Behav 15(4): |
| | | 1744347. |
| OsPrx114 | Disease resistance | Wally, O. and Z. K. Punja (2010). “Enhanced disease |
| | | resistance in transgenic carrot (Daucus carota L.) |
| | | plants over-expressing a rice cationic peroxidase.” |
| | | Planta 232(5): 1229-1239. |
| OsWRKY89 | Disease resistance | Wang, H., et al. (2007). “Overexpression of rice |
| | | WRKY89 enhances ultraviolet B tolerance and |
| | | disease resistance in rice plants.” Plant Mol Biol |
| | | 65(6): 799-815. |
| miR528 | Virus resistance | Shengze, et al. (2019). Transcriptional regulation of |
| | | mir528 by OsSPL9 orchestrates antiviral response in |
| | | rice. [symbol] 1114-1122. |
| OsCDPK1 | Disease resistance | He, S. L., et al. (2018). “Overexpression of a |
| | | constitutively active truncated form of OsCDPK1 |
| | | confers disease resistance by affecting OsPR10a |
| | | expression in rice.” Sci Rep 8(1): 403. |
| OsPR10a | Bacterial leaf blight | Huang, L. F., et al. (2016). “Multiple Patterns of |
| | and streak disease | Regulation and Overexpression of a |
| | resistance | Ribonuclease-Like Pathogenesis-Related Protein |
| | | Gene, OsPR10a, Conferring Disease Resistance in |
| | | Rice and Arabidopsis.” PLoS One 11(6): e0156414. |
| OsWRKY11 | Bacterial leaf blight | Lee, H., et al. (2018). “Rice WRKY11 Plays a Role in |
| | resistance | Pathogen Defense and Drought Tolerance.” Rice |
| | | (NY) 11(1): 5. |
| OsPUB41 | Bacterial leaf blight | Kachewar, N. R., et al. (2019). “Overexpression of |
| | resistance | OsPUB41, a Rice E3 ubiquitin ligase induced by cell |
| | | wall degrading enzymes, enhances immune responses |
| | | in Rice and Arabidopsis.” BMC Plant Biol 19(1): 530. |
| OsFWL5 | Bacterial leaf blight | Li, B., et al. (2019). “Overexpression a “fruit-weight |
| | resistance | 2.2-like” gene OsFWL5 improves rice resistance.” |
| | | Rice (NY) 12(1): 51. |
| OsCM | Bacterial leaf blight | Jan, R., et al. (2020). “Overexpression of OsCM |
| | resistance | alleviates BLB stress via phytohormonal accumulation |
| | | and transcriptional modulation of defense-related |
| | | genes in Oryza sativa.” Sci Rep 10(1): 19520. |
| OsMlo2 | Powdery mildew | Elliott, C., et al. (2002). “Functional conservation of |
| | resistance | wheat and rice Mlo orthologs in defense modulation to |
| | | the powdery mildew fungus.” Mol Plant Microbe |
| | | Interact 15(10): 1069-1077. |
| SE5 | Flowering | Izawa, T., et al. (2000). “Phytochromes confer the |
| | | photoperiodic control of flowering in rice (a short-day |
| | | plant).” Plant J 22(5): 391-399. |
| OsMADS50 | Flowering | Lee, S., et al. (2004). “Functional analyses of the |
| | | flowering time gene OsMADS50, the putative |
| | | SUPPRESSOR OF OVEREXPRESSION OF CO |
| | | 1/AGAMOUS-LIKE 20 (SOC1/AGL20) ortholog in |
| | | rice.” Plant J 38(5): 754-764. |
| OsMADS1 | Flowering | Jeon, J. S., et al. (2000). “leafy hull sterile1 is a |
| | | homeotic mutation in a rice MADS box gene affecting |
| | | rice flower development.” Plant Cell 12(6): 871-884. |
| OsGI | Flowering | Hayama, R., et al. (2003). “Adaptation of |
| | | photoperiodic control pathways produces short-day |
| | | flowering in rice.” Nature 422(6933): 719-722. |
| OsGR2 | Detoxication | Zhang, Z., et al.(2020). Two glyoxylate reductase |
| | | isoforms are functionally redundant but required |
| | | under high photorespiration conditions in rice. BMC |
| | | Plant Biology, 20(1). |
| OsGR1 | Detoxication | Zhang, Z., et al.(2020). Two glyoxylate reductase |
| | | isoforms are functionally redundant but required |
| | | under high photorespiration conditions in rice. BMC |
| | | Plant Biology, 20(1). |
| OsD-LDH2 | To relieve biotic stress | Jain, M., et al. (2020). “A D-lactate dehydrogenase |
| | tolerance | from rice is involved in conferring tolerance to |
| | | multiple abiotic stresses by maintaining cellular |
| | | homeostasis.” Sci Rep 10(1): 12835. |
| OsRab6a | Environment singal | Yang, A., et al. (2016). “A Small GTPase, OsRab6a, |
| | (iron) response | is Involved in the Regulation of Iron Homeostasis in |
| | | Rice.” Front Plant Sci 11: 595439. |
| OsGGP | Overexpression on | Broad, R. C., et al. (2020). “Effect of Rice |
| | ascorbate | GDP-L-Galactose Phosphorylase Constitutive |
| | concentration; stress | Overexpression on Ascorbate Concentration, Stress |
| | tolerance | Tolerance, and Iron Bioavailability in Rice.” Front |
| | | Plant Sci 11: 595439. |
| OsMADS58 | Floral organ | Ming, Zheng ., et al.(2015). “DEFORMED FLORAL |
| | development | ORGAN1 (DFO1) regulates floral organ identity by |
| | | epigenetically repressing the expression of |
| | | OsMADS58 in rice (Oryza sativa).” New Phytologist, |
| | | 206(4). |
| DFO1 | Floral organ | Ming, Zheng., et al.(2015). “DEFORMED FLORAL |
| | development | ORGAN1 (DFO1) regulates floral organ identity by |
| | | epigenetically repressing the expression of |
| | | OsMADS58 in rice (Oryza sativa).” New Phytologist, |
| | | 206(4). |
| OsUCL23 | Pollen development | Zhang, Y. C., et al.(2019). “OsmiR528 regulates |
| | | rice-pollen intine formationby targeting an uclacyanin |
| | | to influence flavonoid metabolism.” Proceedings of the |
| | | National Academy of Sciences, 117(1), 201810968. |
| allene oxide | Brown planthopper | Chu, H., et al. (2013). “A CLE-WOX signalling |
| cyclase | resistance | module regulates root meristem maintenance and |
| | | vascular tissue development in rice.” J Exp Bot |
| | | 64(17): 5359-5369. |
| Os6PGDH1 | Brown planthopper | Chen, L., et al. (2020). “Overexpression of a Cytosolic |
| | | 6-Phosphogluconate Dehydrogenase Gene Enhances |
| | | the Resistance of Rice to Nilaparvata lugens.” Plants |
| | | (Basel) 9(11). |
| OsGID1 | Brown planthopper | Chen, L., et al. (2018). “Overexpression of OsGID1 |
| | | Enhances the Resistance of Rice to the Brown |
| | | Planthopper Nilaparvata lugens.” Int J Mol Sci 19(9). |
| OsSPL7 | Hydrogen peroxide | Hoang, T. V., et al. (2019). “Heat stress transcription |
| | accumulation, rice | factor OsSPL7 plays a critical role in reactive oxygen |
| | blast resistance, | species balance and stress responses in rice.” Plant Sci |
| | bacterial blight | 289: 110273. |
| | resistance, cold | |
| | tolerance | |
| OsNAS2 | zinc and ironconten | Moreno-Moyano, L. T., et al.(2016). “Association of |
| | in rice gains | Increased Grain Iron and Zinc Concentrations with |
| | | Agro-morphological Traits ofBiofortified |
| | | Rice.” Frontiers in Plant Science, 7, 1463. |
| OsGrxC2.2 | increasegrain weight | Liu, S., et al. (2019). “Overexpression of a |
| | | CPYC-Type Glutaredoxin, OsGrxC2.2, Causes |
| | | Abnormal Embryos and an Increased Grain Weight in |
| | | Rice” |
| OsBBI1 | broad spectrum | Wei Li., et al.(2011). “Rice RING protein OsBBI1 |
| | resistance to | with E3 ligase activity confers broad-spectrum |
| | Magnaporthe oryzae | resistance against Magnaporthe oryzae by modifying |
| | | the cell wall defence.” Cell Research, 21(5), 835-848. |
| SDG711 | large inflorescence | Liu, X., et al. (2015). “Regulation of Histone |
| | | Methylation and Reprogramming of Gene Expression |
| | | in the Rice Inflorescence Meristem.” Plant Cell, 27(5), |
| | | 1428-44. |
| PMM1 | increaseyield and | Li, Y., et al.(2018). “Panicle Morphology Mutant 1 |
| | grain weight | (PMM1) determines the inflorescence architecture of |
| | | rice by controlling brassinosteroid biosynthesis.” BMC |
| | | Plant Biology, 18(1). |
| SDG701 | early flowering | Liu, K. P., et al.(2017). “SET DOMAIN GROUP701 |
| | | encodes a H3K4-methytransferase and regulates |
| | | multiple key processes of rice plant |
| | | development.” NEW PHYTOL, 2017,215(2)(—), |
| | | 609-623. |
| SAPK10 | early flowering | Xixi, Liu., et al. (2019). “Protein Interactomic |
| | | Analysis of SAPKs and ABA-Inducible bZIPs |
| | | Revealed Key Roles of SAPK10 in Rice |
| | | Flowering.” International Journal of Molecular |
| | | Sciences. |
| OsPAP10c | phosphorus utilization | Lu, L., et al. (2016). “OsPAP10c, a novel secreted |
| | | acid phosphatase in rice, plays an important role in the |
| | | utilization of external organic phosphorus.” Plant, Cell |
| | | & Environment. |
| OsHsfA7 | salt and drought | Liu, A. L., et al. (2013). “Over-expression of |
| | tolerance | OsHsfA7 enhanced salt and drought tolerance in |
| | | transgenic rice.” BMB Reports, 46(1). |
| OsMYB3R-2 | cold tolerance | Qibin Ma., et al.(2009). “Enhanced T olerance to |
| | | Chilling Stress in OsMYB3R-2T ransgenic Rice Is |
| | | Mediated by Alteration in Cell Cycle and Ectopic |
| | | Expression of Stress Genes.” Plant Physiology, |
| | | 150(1), 244-256. |
| OsDREB1G | cold tolerance | Moon, S. J., et al.(2019). “Ectopic Expression of |
| | | OsDREB1G, a Member of the OsDREB1 Subfamily, |
| | | Confers Cold Stress Tolerance in Rice.” Frontiers in |
| | | plant science, 10. |
| SAPK1 | salt tolerance | Lou, D., et al.(2018). “The sucrose |
| SAPK2 | | non-fermenting-1-related protein kinases SAPK1 and |
| | | SAPK2 function collaboratively as positive regulators |
| | | of saltstress tolerance in rice.” BMC Plant Biology, |
| | | 18(1). |
| OsRacB | salt tolerance | Min, L., et al.(2010). Rice gtpase osracb: potential |
| | | accessory factor in plant salt-stress signaling. Acta |
| | | Biochimica Et Biophysica Sinica, 38(6), 393-402. |
| OsCNX6 | salt tolerance | Xin Liu., et al.(2018). Identification and |
| | | characterization of the rice pre-harvest sprouting |
| | | mutants involved in molybdenum cofactor |
| | | biosynthesis. The New Phytologist. |
| OsCIPK30 | tolerance to rice | Liu, Y., et al. (2020). “Overexpression of OsCIPK30 |
| | stripe virus | Enhances Plant T olerance to Rice stripe |
| | | virus” Frontiers in Microbiology, 8, 2322. |
| OsGAPB | low light | “Proteomic Analysis of Rice Subjected to Low Light |
| | stresstolerance | Stress and Overexpression of OsGAPB Increases the |
| | | Stress Tolerance.” Rice, 13(1). |
| OsERF3 | stress resistance | Jing, L., et al. (2011). “An EAR-motif-containing ERF |
| | | transcription factor affects herbivore-induced |
| | | signaling, defense and resistance in rice.” Plant |
| | | Journal, 68(4), 583-596. |
| OsARD1 | stress resistance | Liang, S., et al. (2019). “Overexpression of OsARD1 |
| | | Improves Submergence, Drought, and Salt Tolerances |
| | | of Seedling Through the Enhancement of Ethylene |
| | | Synthesis in Rice.” Frontiers in Plant Science, 10. |
| OsDHODH1 | drought and salt | Liu, W. Y., et al.(2009). “The OsDHODH1 Gene is |
| | tolerance | Involved in Salt and Drought Tolerance in |
| | | Rice.” Journal of Integrative Plant Biology, 51(009), |
| | | 825-833. |
| RWC3 | drought tolerance | Lian, H. L., et al.(2004). “The Role of Aquaporin |
| | | RWC3 in Drought A voidance in Rice.” Plant & Cell |
| | | Physiology. |
| bZIP73 | cold tolerance | Liu, C., et al. (2019). “The bZIP73 transcription factor |
| | | controls rice cold tolerance at the reproductive |
| | | stage” Plant Biotechnology Journal, 17(9). |
| OsPdk1 | rice blast disease | Hirochika, H., et al. (2010). “Pdk1 Kinase Regulates |
| | resistance and | Basal Disease Resistance Through the |
| | bacterial leaf blight | OsOxi1-OsPti1a Phosphorylation Cascade in |
| | disease | Rice.” Plant & Cell Physiology, 51(12), 2082-91. |
| OsCBSX3 | rice blast disease | Mou, S., et al.(2015). Over-expression of rice CBS |
| | resistance | domain containing protein, OsCBSX3, confers rice |
| | | resistance to magnaporthe oryzae inoculation. |
| | | International Journal of Molecular Sciences, 16(7), |
| | | 15903-15917. |
| OsAAA-ATPase1 | rice blast disease | Liu, X., et al. (2020). Rice OsAAA-ATPase1 is |
| | resistance | induced during blast infection in a salicylic |
| | | acid-dependent manner, and promotes blast fungus |
| | | resistance. International Journal of Molecular |
| | | Sciences, 21(4), 1443. |
| XA3 | bacterial leaf blight | Liu, F., et al. (2020). The rice xa3 gene confers |
| | disease resistance | resistance to xanthomonas oryzae pv. oryzae in the |
| | | model rice kitaake genetic background. Frontiers in |
| | | Plant Science, 11. |
| OsTGA2 | bacterial leaf blight | Seok-Jun., et al.(2018). OsTGA2 confers disease |
| | disease resistance | resistance to rice against leaf blight by regulating |
| | | expression levels of disease related genes via |
| | | interaction with NH1. PloS one, 13(11), e0206910. |
| OsCYP71Z2 | bacterial leaf blight | Wenqi, L., et al.(2019). Overexpressing CYP71z2 |
| | disease resistance | enhances resistance to bacterial blight by suppressing |
| | | auxin biosynthesis in rice. PloS one. |
| OsPIANK1 | bacterial leaf blight | Mou, S., et al., (2013). Functional analysis and |
| | diseaseresistance | expressional characterization of rice ankyrin |
| | | repeat-containing protein, ospiank1, in basal defense |
| | | against magnaporthe oryzae attack. Plos One, 8. |
| NOE1/OsCATC | programmed cell death | Runlong, M., et al.(2013). Nitric Oxide and Protein |
| | | S-Nitrosylation Are Integral to Hydrogen |
| | | Peroxide-Induced Leaf Cell Death in Rice |
| OsEXPA8 | facilitating cell | Ma, N., et al, (2013). Overexpression of OsEXPA8, a |
| | extension | root-specific gene, improves rice growth and root |
| | | system architecture by facilitating cell extension. |
| | | PLOS ONE, 8(10), e75997-. |
| OsSnRK1a | Broad spectrum | Filipe, O., et al. (2018). “The energy sensor |
| | disease resistance | OsSnRK1a confers broad-spectrum disease resistance |
| | (rice blast) | in rice.” Sci Rep 8(1): 3864. |
| OsMADS25 | Root development | Ning Xu., et al.(2018). Rice transcription factor |
| | | OsMADS25 modulates root growth and confers |
| | | salinity tolerance via the ABA-mediated regulatory |
| | | pathway and ROS scavenging. PLoS genetics. |
| MHZ5 | Root and coleoptile | Yin, C. C., et al. (2015). Ethylene responses in rice |
| | growth | roots and coleoptiles are differentially regulated by a |
| | | carotenoid isomerase-mediated abscisic acid pathway. |
| | | Plant Cell, 27(4), 1061-1081. |
| OsPIN1 | Root development and | Xu, M., et al.(2005)A pin1 family gene, ospin1, |
| | tillering | involved in auxin-dependent adventitious root |
| | | emergence and tillering in rice. Plant & Cell |
| | | Physiology (10), 1674. |
| OsRDCP1 | Drought stress | Bae, H., et al. (2011). “Overexpression of OsRDCP1, |
| | tolerance | a rice RING domain-containing E3 ubiquitin ligase, |
| | | increased tolerance to drought stress in rice (Oryza |
| | | sativa L.).” Plant Sci 180(6): 775-782. |
| OsRPK1 | Negatively regulate | Zou, Y., et al. (2014). “OsRPK1, a novel leucine-rich |
| | plant height and tiller | repeat receptor-like kinase, negatively regulates polar |
| | number | auxin transport and root development in rice.” |
| | | Biochim Biophys Acta 1840(6): 1676-1685. |
| ONAC095 | Negatively regulates | Huang, L., et al. (2016). “Rice NAC transcription |
| | drought tolerance and | factor ONAC095 plays opposite roles in drought and |
| | positively regulates | cold stress tolerance.” BMC Plant Biol 16(1): 203. |
| | cold tolerance | |
| OsAAP4 | Tillering and yield | Fang, Z., et al. (2021). “The Amino Acid Transporter |
| | | OsAAP4 Contributes to Rice Tillering and Grain |
| | | Yield by Regulating Neutral Amino Acid Allocation |
| | | through Two Splicing Variants.” Rice (NY) 14(1): 2. |
| OsRAN2 | Abiotic stress | Zang, A., et al. (2010). Overexpression of OsRAN2 in |
| | | rice and Arabidopsis renders transgenic plants |
| | | hypersensitive to salinity and osmotic stress. Journal |
| | | of experimental botany, 61(3), 777-789. |
| OsCYP71D8L | Abiotic stress | Zhou, J., et al (2020). CYP71D8L is a key regulator |
| | | involved in growth and stress responses by mediating |
| | | gibberellin homeostasis in rice. Journal of |
| | | experimental botany, 71(3), 1160-1170. |
| OsTRXh1 | Development and | Zhang, C. et al (2011). An apoplastic h-type |
| | stress response | thioredoxin is involved in the stress response through |
| | | regulation of the apoplastic reactive oxygen species in |
| | | rice. Plant Physiology, 157(4), 1884-1899. |
| RF2a | To exert strong | Petruccelli, S., et al. (2001). “Transcription factor |
| | negative effect on the | RF2a alters expression of the rice tungro bacilliform |
| | development of | virus promoter in transgenic tobacco plants.” Proc |
| | transgenic plants | Natl Acad Sci USA 98(13): 7635-7640. |
| OsPM1 | Response to drought | Yao, L., et al. (2018). The AWPM-19 family protein |
| | | OsPM1 mediates abscisic acid influx and drought |
| | | response in rice. The Plant Cell, 30(6), 1258-1276. |
| OsCO3 | To delay flowering in | Kim, S. K., et al. (2008). “OsCO3, a |
| | the conditions of short | CONSTANS-LIKE gene, controls flowering by |
| | daylight | negatively regulating the expression of FT-like genes |
| | | under SD conditions in rice.” Planta 228(2): 355-365 |
| OsLsi1 | Resistance to cold | Fang, C., et al. (2017). “Overexpression of Lsi1 in |
| | stress | cold-sensitive rice mediates transcriptional regulatory |
| | | networks and enhances resistance to chilling stress.” |
| | | Plant Sci 262: 115-126. |
| Perox3 | Rice blast resistance | Zhu, Z., et al. (2020). “New insights into |
| | | bsr-d1-mediated broad-spectrum resistance to rice |
| | | blast.” Mol Plant Pathol 21(7): 951-960. |
| OsWRKY53 | Rice blast resistance | Chujo, T., et al. (2014). “Overexpression of |
| | | phosphomimic mutated OsWRKY53 leads to |
| | | enhanced blast resistance in rice.” PLoS One 9(6): |
| | | e98737. |
| OsSPK1 | Rice blast resistance | Wang, Q., et al. (2018). “Resistance protein Pit |
| | | interacts with the GEF OsSPK1 to activate OsRac1 |
| | | and trigger rice immunity.” Proc Natl Acad Sci USA |
| | | 115(49): E11551-E11560. |
| OsDjA9 | Rice blast resistance | Xu, G., et al. (2020). “A fungal effector targets a heat |
| | | shock-dynamin protein complex to modulate |
| | | mitochondrial dynamics and reduce plant immunity.” |
| | | Sci Adv 6(48). |
| OsCPK4 | Rice blast resistance | Bundo, M. and M. Coca (2016). “Enhancing blast |
| | | disease resistance by overexpression of the |
| | | calcium-dependent protein kinase OsCPK4 in rice.” |
| | | Plant Biotechnol J 14(6): 1357-1367. |
| Osa-miR162 | Rice blast resistance | Li, X. P., et al. (2020). “Osa-miR162a fine-tunes rice |
| | | resistance to Magnaporthe oryzae and Yield.” Rice |
| | | (NY) 13(1): 38. |
| APIP4 | Rice blast | Zhang, C., et al. (2020). “A fungal effector and a rice |
| | | NLR protein have antagonistic effects on a |
| | | Bowman-Birk trypsin inhibitor.” Plant Biotechnol J |
| | | 18(11): 2354-2363. |
| OsTPS19 | Rice blast | Chen, X., et al. (2018). “The rice terpene synthase |
| | | gene OsTPS19 functions as an (S)-limonene synthase |
| | | in planta, and its overexpression leads to enhanced |
| | | resistance to the blast fungus Magnaporthe oryzae.” |
| | | Plant Biotechnol J 16(10): 1778-1787. |
| RBBI2-3 | Rice blast | Qu, L. J., et al. (2003). “Molecular cloning and |
| | | functional analysis of a novel type of Bowman-Birk |
| | | inhibitor gene family in rice.” Plant Physiol 133(2): |
| | | 560-570. |
| TLH | Rice blast | Rehmeyer, C., et al. (2006). “Organization of |
| | | chromosome ends in the rice blast fungus, |
| | | Magnaporthe oryzae.” Nucleic Acids Res 34(17): |
| | | 4685-4701. |
| Rirlb | Rice blast | Schaffrath, U., et al. (2000). “Constitutive expression |
| | | of the defense-related Rir1b gene in transgenic rice |
| | | plants confers enhanced resistance to the rice blast |
| | | fungus Magnaporthe grisea.” Plant Mol Biol 43(1): |
| | | 59-66. |
| MoSDT1 | Rice blast | Wang, C., et al. (2019). “Overexpression of |
| | | Magnaporthe Oryzae Systemic Defense Trigger 1 |
| | | (MoSDT1) Confers Improved Rice Blast Resistance in |
| | | Rice.” Int J Mol Sci 20(19). |
| Met6 | Rice blast | Saint-Macary, M. E., et al. (2015). “Methionine |
| | | biosynthesis is essential for infection in the rice blast |
| | | fungus Magnaporthe oryzae.” PLoS One 10(4): |
| | | e0111108. |
| Cpk2 | Rice blast | Selvaraj, P., et al. (2017). “Cpk2, a Catalytic Subunit |
| | | of Cyclic AMP-PKA, Regulates Growth and |
| | | Pathogenesis in Rice Blast.” Front Microbiol 8: 2289. |
| APIP12 | Rice blast | Tang, M., et al. (2017). “The Nup98 Homolog APIP12 |
| | | Targeted by the Effector AvrPiz-t is Involved in Rice |
| | | Basal Resistance Against Magnaporthe oryzae.” Rice |
| | | (NY) 10(1): 5. |
| SEP1 | Rice blast | Saunders, D. G., et al. (2010). “Spatial uncoupling of |
| | | mitosis and cytokinesis during appressorium-mediated |
| | | plant infection by the rice blast fungus Magnaporthe |
| | | oryzae.” Plant Cell 22(7): 2417-2428. |
| OsATG8a | Nitrogen use | Yu, J., et al (2019). Increased autophagy of rice can |
| | efficiency | increase yield and nitrogen use efficiency (NUE). |
| | | Frontiers in plant science, 10, 584. |
| RDD1 | To improve nutrient | Iwamoto, M. and A. Tagiri (2016). |
| | uptake and increase | “MicroRNA-targeted transcription factor gene RDD1 |
| | yield | promotes nutrient ion uptake and accumulation in |
| | | rice.” Plant J 85(4): 466-477. |
| OsCCT19 | Head sprouting | Zhang, L., et al (2015). Three CCT domain-containing |
| | | genes were identified to regulate heading date by |
| | | candidate gene-based association mapping and |
| | | transformation in rice. Scientific reports, 5(1), 1-11. |
| OsCCT11 | Head sprouting | Zhang, L., et al (2015). Three CCT domain-containing |
| | | genes were identified to regulate heading date by |
| | | candidate gene-based association mapping and |
| | | transformation in rice. Scientific reports, 5(1), 1-11. |
| OsCCT01 | Head sprouting | Zhang, L., et al (2015). Three CCT domain-containing |
| | | genes were identified to regulate heading date by |
| | | candidate gene-based association mapping and |
| | | transformation in rice. Scientific reports, 5(1), 1-11. |
| SLRL1 | Gibberellin signaling | Itoh, H., et al. (2005). “Overexpression of a GRAS |
| | repressor | protein lacking the DELLA domain confers altered |
| | | gibberellin responses in rice.” Plant J 44(4): 669-679. |
| OsGIF1 | Yield and plant | He, Z., et al. (2017). “OsGIF1 Positively Regulates |
| | morphology | the Sizes of Stems, Leaves, and Grains in Rice.” Front |
| | | Plant Sci 8: 1730. |
| OsDHAR1 | Yield and biomass | Kim, Y. S., et al. (2013). “Homologous expression of |
| | | cytosolic dehydroascorbate reductase increases grain |
| | | yield and biomass under paddy field conditions in |
| | | transgenic rice (Oryza sativa L. japonica).” Planta |
| | | 237(6): 1613-1625. |
| WG7 | Yield | Huang, Y., et al. (2020). “Wide Grain 7 increases |
| | | grain width by enhancing H3K4me3 enrichment in the |
| | | OsMADS1 promoter in rice (Oryza sativa L.).” Plant J |
| | | 102(3): 517-528. |
| PDH45 | Yield | Sahoo, R. K., et al. (2012). “Pea DNA helicase 45 |
| | | promotes salinity stress tolerance in IR64 rice with |
| | | improved yield.” Plant Signal Behav 7(8): 1042-1046. |
| OsSUS3 | Yield | Fan, C., et al. (2019). “Sucrose Synthase Enhances |
| | | Hull Size and Grain Weight by Regulating Cell |
| | | Division and Starch Accumulation in Transgenic |
| | | Rice.” Int J Mol Sci 20(20). |
| OsSND2 | Yield | Ye, Y., et al (2018). OsSND2, aNAC family |
| | | transcription factor, is involved in secondary cell wall |
| | | biosynthesis through regulating MYBs expression in |
| | | rice. Rice, 11(1), 1-14. |
| OsSGL | Yield | Wang, M., et al. (2016). “OsSGL, a novel pleiotropic |
| | | stress-related gene enhances grain length and yield in |
| | | rice.” Sci Rep 6: 38157. |
| OsRac1 | Yield | Zhang, Y., et al (2019). The Rho-family GTPase |
| | | OsRac1 controls rice grain size and yield by |
| | | regulating cell division. Proceedings of the National |
| | | Academy of Sciences, 116(32), 16121-16126 |
| OsqLL9 | Yield | Fu, X., et al. (2019). “Enhanced Expression of QTL |
| | | qLL9/DEP1 Facilitates the Improvement of Leaf |
| | | Morphology and Grain Yield in Rice.” Int J Mol Sci |
| | | 20(4). |
| OsNRT2.3b | Yield | Fan, X., et al. (2016). “Overexpression of a |
| | | pH-sensitive nitrate transporter in rice increases crop |
| | | yields.” Proc Natl Acad Sci USA 113(26): |
| | | 7118-7123. |
| OsNPF7.2 | Yield | Wang, J., et al. (2018). “Rice nitrate transporter |
| | | OsNPF7.2 positively regulates tiller number and grain |
| | | yield.” Rice (NY) 11(1): 12. |
| OsNLP4 | Yield | Wu, J.,et al (2021). Rice NIN-LIKE PROTEIN 4 plays |
| | | a pivotal role in nitrogen use efficiency. Plant |
| | | biotechnology journal, 19(3), 448-461. |
| OsMYB103L | Yield | Yang, C., et al(2014). OsMYB103L, an R2R3-MYB |
| | | transcription factor, influences leaf rolling and |
| | | mechanical strength in rice (Oryza sativa L.). BMC |
| | | plant biology, 14(1), 1-15. |
| OsMPH1 | Yield | Zhang, Y., et al(2017). OsMPH1 regulates plant |
| | | height and improves grain yield in rice. PLoS one, |
| | | 12(7), e0180825. |
| OsmiR156 | Yield | Zhao, M., et al (2015). Regulation of OsmiR156h |
| | | through alternative polyadenylation improves grain |
| | | yield in rice. PloS one, 10(5), e0126154. |
| OsLSK1 | Yield | Zou, X., et al. (2015). “Over-expression of an |
| | | S-domain receptor-like kinase extracellular domain |
| | | improves panicle architecture and grain yield in rice.” |
| | | J Exp Bot 66(22): 7197-7209. |
| OsEATB | Yield | Qi, W., et al. (2011). “Rice ethylene-response |
| | | AP2/ERF factor OsEATB restricts internode |
| | | elongation by down-regulating a gibberellin |
| | | biosynthetic gene.” Plant Physiol 157(1): 216-228. |
| OsCu/Zn-SOD | Yield | Guan, Q., et al. (2017). “Tolerance analysis of |
| | | chloroplast OsCu/Zn-SOD overexpressing rice under |
| | | NaCl and NaHCO3 stress.” PLoS One 12(10): |
| | | e0186052. |
| OsbHLH107 | Yield | Yang, X., et al (2018). Overexpression of |
| | | OsbHLH107, a member of the basic helix-loop-helix |
| | | transcription factor family, enhances grain size in rice |
| | | (Oryza sativa L.). Rice, 11(1), 1-12. |
| OsbHLH079 | Yield | Seo, H., et al. (2020). “The Rice Basic |
| | | Helix-Loop-Helix 79 (OsbHLH079) Determines Leaf |
| | | Angle and Grain Shape.” Int J Mol Sci 21(6). |
| OsATG8c | Yield | Zhen, X., et al(2019). OsATG8c-mediated increased |
| | | autophagy regulates the yield and nitrogen use |
| | | efficiency in rice. International journal of molecular |
| | | sciences, 20(19), 4956. |
| OsASN1 | Yield | Lee, S., et al. (2020). “OsASN1 Overexpression in |
| | | Rice Increases Grain Protein Content and Yield under |
| | | Nitrogen-Limiting Conditions.” Plant Cell Physiol |
| | | 61(7): 1309-1320. |
| OsAP2-39 | Yield | Yaish, M. W., et al(2010). The APETALA-2-like |
| | | transcription factor OsAP2-39 controls key |
| | | interactions between abscisic acid and gibberellin in |
| | | rice. PLoS Genet, 6(9), el001098. |
| OsAFB6 | Yield | He, Q., et al. (2018). “Overexpression of an auxin |
| | | receptor OsAFB6 significantly enhanced grain yield |
| | | by increasing cytokinin and decreasing auxin |
| | | concentrations in rice panicle.” Sci Rep 8(1): 14051. |
| OsACBP2 | Yield | Guo, Z. H., et al. (2019). “The overexpression of rice |
| | | ACYL-CoA-BINDING PROTEIN2 increases grain |
| | | size and bran oil content in transgenic rice.” Plant J |
| | | 100(6): 1132-1147. |
| OsABCG18 | Yield | Zhao, J.,et al (2019). ABC transporter OsABCG18 |
| | | controls the shootward transport of cytokinins and |
| | | grain yield in rice. Journal of experimental botany, |
| | | 70(21), 6277-6291. |
| OsAAP6 | Yield | Ji, Y., et al. (2020). “The amino acid transporter |
| | | AAP1 mediates growth and grain yield by regulating |
| | | neutral amino acid uptake and reallocation in Oryza |
| | | sativa.” J Exp Bot 71(16): 4763-4777. |
| OSA1 | Yield | Zhang, M., et al (2021). Plasma membrane H+-ATPase |
| | | overexpression increases rice yield via simultaneous |
| | | enhancement of nutrient uptake and photosynthesis. |
| | | Nature communications, 12(1), 1-12. |
| NOG1 | Yield | Huo, X., et al. (2017). “NOG1 increases grain |
| | | production in rice.” Nat Commun 8(1): 1497. |
| miR1432 | Yield | Zhao, Y. F., et al(2019). “miR1432-OsACOT |
| | | (Acyl-CoA thioesterase) module determines grain |
| | | yield via enhancing grain filling rate in rice.” Plant |
| | | biotechnology journal, 17(4), 712-723. |
| LRK1 | Yield | Zha, X.,et.al (2009). “Overexpression of the rice |
| | | LRK1 gene improves quantitative yield components.” |
| | | Plant biotechnology journal, 7(7), 611-620. |
| LAIR | Yield | Wang, Y., et al (2018). “Overexpressing IncRNA |
| | | LAIR increases grain yield and regulates |
| | | neighbouring gene cluster expression in rice.” Nature |
| | | communications, 9(1), 1-9. |
| HD1 | Yield | Piao, R., et al. (2014). “Isolation and characterization |
| | | of a dominant dwarf gene, d-h, in rice.” PLoS One |
| | | 9(2): e86210. |
| GW6 | Yield | Shi, C. L., et al. (2020). “A quantitative trait locus |
| | | GW6 controls rice grain size and yield through the |
| | | gibberellin pathway.” Plant J 103(3): 1174-1188. |
| GNS4 | Yield | Zhou, Y., et al (2017). “GNS4, a novel allele of |
| | | DWARF11, regulates grain number and grain size in a |
| | | high-yield rice variety.” Rice, 10(1), 1-11. |
| DEGs | Yield | Swamy, B. P., et al. (2013). “Genetic, physiological, |
| | | and gene expression analyses reveal that multiple |
| | | QTL enhance yield of rice mega-variety IR64 under |
| | | drought.” PLoS One 8(5): e62795. |
| CPB1/D11 | Yield | Wu, Y., et al (2016). “CLUSTERED PRIMARY |
| | | BRANCH 1, a new allele of DWARF 11, controls |
| | | panicle architecture and seed size in rice.” Plant |
| | | biotechnology journal, 14(1), 377-386. |
| Big Grain 1 | Yield | Liu, L., et al. (2015). “Activation of big grain1 |
| | | significantly improves grain size by regulating auxin |
| | | transport in rice.” Proc Natl Acad Sci USA. |
| | | 112: 11102-11107. |
| AtGolS2 | Yield | Selvaraj, M. G., et al. (2017). “Overexpression of an |
| | | Arabidopsis thaliana galactinol synthase gene |
| | | improves drought tolerance in transgenic rice and |
| | | increased grain yield in the field.” Plant Biotechnol J |
| | | 15(11): 1465-1477. |
| OsNLP1(NIN-LIKE | Yield | Alfatih, A., et al. (2020). “Rice NIN-LIKE PROTEIN |
| PROTEIN 1) | | 1 rapidly responds to nitrogen deficiency and |
| | | improves yield and nitrogen use efficiency.” J Exp |
| | | Bot 71(19): 6032-6042. |
| OsNHX1 | Yield | Qu, M., et al. (2020). “Alterations in stomatai |
| | | response to fluctuating light increase biomass and |
| | | yield of rice under drought conditions.” Plant J |
| | | 104(5): 1334-1347. |
| CYP734A4 | Yield | Qian, W., et al. (2017). “Novel rice mutants |
| | | overexpressing the brassinosteroid catabolic gene |
| | | CYP734A4.” Plant Mol Biol 93(1-2): 197-208. |
| AtICE1 | Yield | Verma, R. K., et al. (2020). “Overexpression of |
| | | Arabidopsis ICE1 enhances yield and multiple abiotic |
| | | stress tolerance in indica rice.” Plant Signal Behav |
| | | 15(11): 1814547. |
| OsGH3.1 | Resistance to a fungal | Domingo, C., et al. (2009). “Constitutive expression |
| | pathogen | of OsGH3.1 reduces auxin content and enhances |
| | | defense response and resistance to a fungal pathogen |
| | | in rice.” Mol Plant Microbe Interact 22(2): 201-210. |
| OsHAP2E | Tolerance to | Alam, M. M., et al. (2015). “Overexpression of a rice |
| | pathogens, salinity | heme activator protein gene (OsHAP2E) confers |
| | and drought | resistance to pathogens, salinity and drought, and |
| | | increases photosynthesis and tiller number.” Plant |
| | | Biotechnol J 13(1): 85-96. |
| Oshox4 | Semi-dwarfing | Dai, M., et al. (2008). “Functional analysis of rice |
| | phenotype | HOMEOBOX4 (Oshox4) gene reveals a negative |
| | | function in gibberellin responses.” Plant Mol Biol |
| | | 66(3): 289-301. |
| EUI | Resistance to bacterial | Yang, D. L., et al (2008). “Altered Disease |
| | leaf blight and leaf | Development in the eui Mutants |
| | blast in rice | and Eui Overexpressors Indicates that Gibberellins |
| | | Negatively Regulate Rice Basal Disease |
| | | Resistance.” Molecular plant, 1(3), 528-537. |
| LRR1 | Resistance to bacterial | Caddell, D. F., et al. (2017). “Silencing of the Rice |
| | leaf blight in rice | Gene LRR1 Compromises Rice Xa21 Transcript |
| | | Accumulation and XA21-Mediated Immunity.” Rice |
| | | (NY) 10(1): 23. |
| GH3-8, | Resistance to bacterial | Ding, X., et al. (2008). “Activation of the |
| | leaf blight in rice | indole-3-acetic acid-amido synthetase GH3-8 |
| | | suppresses expansin expression and promotes |
| | | salicylate- and jasmonate-independent basal immunity |
| | | in rice.” Plant Cell 20(1): 228-240. |
| OsSWEET14 | Resistance to bacterial | Tran, T. T., et al. (2018). “Functional analysis of |
| | leaf blight in rice | African Xanthomonas oryzae pv. oryzae TALomes |
| | | reveals a new susceptibility gene in bacterial leaf |
| | | blight of rice.” PLoS Pathog 14(6): e1007092. |
| cecropin B | Resistance to bacterial | Sharma, A., et al. (2000). “Transgenic expression of |
| | leaf blight in rice | cecropin B, an antibacterial peptide from Bombyx |
| | | mori, confers enhanced resistance to bacterial leaf |
| | | blight in rice.” FEBS Lett 484(1): 7-11. |
| OsAMT1-1 | Ammonium uptake | Hoque, M. S., et al. (2006). “Over-expression of the |
| | | rice OsAMT1-1 gene increases ammonium uptake and |
| | | content, but impairs growth and development of plants |
| | | under high ammonium nutrition.” Funct Plant Biol |
| | | 33(2): 153-163. |
| OsWRKY36 | Dwarfing | Lan, J., et al. (2020). “Small grain and semi-dwarf 3, a |
| | | WRKY transcription factor, negatively regulates plant |
| | | height and grain size by stabilizing SLR1 expression |
| | | in rice.” Plant Mol Biol 104(4-5): 429-450 |
| bHLH6 | Pi uptake | He, Q., et al. (2020). “OsbHLH6 interacts with |
| | | OsSPX4 and regulates the phosphate starvation |
| | | response in rice.” Plant J. |
| OsNPF7.7 | Nitrogen uptake and | Huang, W., et al. (2018). “Two Splicing Variants of |
| | utilization | OsNPF7.7 Regulate Shoot Branching and Nitrogen |
| | | Utilization Efficiency in Rice.” Front Plant Sci 9: 300. |
| OsIMA1 | Fe uptake | Kobayashi, T., et al. (2020). “Iron |
| | | deficiency-inducible peptide coding genes OsIMA1 |
| | | and OsIMA2 positively regulate a major pathway of |
| | | iron uptake and translocation in rice.” J Exp Bot. |
| SAPK9 | Drought tolerance and | Dey, A., et al. (2016). “The sucrose non-fermenting |
| | grain yield | 1-related kinase 2 gene SAPK9 improves drought |
| | | tolerance and grain yield in rice by modulating |
| | | cellular osmotic potential, stomatai closure and |
| | | stress-responsive gene expression.” BMC Plant Biol |
| | | 16(1): 158. |
| DNG701 | DNA methylation | La, H., et al. (2011). “A 5-methylcytosine DNA |
| | | glycosylase/lyase demethylates the retrotransposon |
| | | Tos17 and promotes its transposition in rice.” Proc |
| | | Natl Acad Sci USA 108(37): 15498-15503. |
| OsSLG | grain yield | Feng, Z., et al. (2016). “SLG controls grain size and |
| | | leaf angle by modulating brassinosteroid homeostasis |
| | | in rice.” J Exp Bot 67(14): 4241-4253. |

Table I lists the representative functional genes in wheat. Methods in the present invention can be used to edit such genes and create new genes by designing new combinations of different gene elements or different protein domain, and can be utilized in wheat breeding program.

TABLE I
Important functional genes in wheat

| Gene name | Application | Reference |
|---|---|---|
| TaDOG1L1 | Seed | Ashikawa I, et al. (2010). Ectopic expression of wheat and barley |
| | dormancy | dog1-like genes promotes seed dormancy in arabidopsis. Plant |
| | | Science An International Journal of Experimental Plant Biology, |
| | | 179(5), 536-542. |
| CTR1 | Salt | Cai-Li B I, et al. (2010). Cloning and characterization of a |
| | resistance | putative Ctrl gene from wheat. Journal of Integrative Agriculture, |
| | | 9(009), 1241-1250. |
| TaPIEP1 | Pathogenic | Dong N, et al. (2010). Overexpression of tapiep1, a |
| | bacteria | pathogen-induced erf gene of wheat, confers host-enhanced |
| | resistance | resistance to fungal pathogen bipolaris sorokiniana. Functional & |
| | | Integrative Genomics, 10(2), 215-226. |
| 1Dy12 | Flour | Valquiria R M Pierucci, et al. (2009). Effects of overexpression |
| | quality | of high molecular weight glutenin subunit 1dy10 on wheat tortilla |
| | | properties. J Agric Food Chern, 57(14), 6318-6326. |
| STRP | Salt | Zhou W, et al. (2009). Overexpression of tastrg gene improves |
| | resistance | salt and drought tolerance in rice. Journal of Plant Physiology, |
| | | 166(15), 1660-1671. |
| TaAIDFa | Signal | Xu Z S, et al. (2008). Characterization of the taaidfa gene |
| | transduction | encoding a crt/dre-binding factor responsive to drought, high-salt, |
| | | and cold stress in wheat. Molecular Genetics & Genomics, |
| | | 280(6), 497-508. |
| Waox1a | Cold | Sugie A, et al. (2006). Overexpression of wheat alternative |
| | tolerance | oxidase gene waox1a alters respiration capacity and response to |
| | | reactive oxygen species under low temperature in transgenic |
| | | arabidopsis. Genes & Genetic Systems, 81(5), 349-354. |
| skp1 | Promotion | Li C, et al. (2006). Cloning and expression analysis of tsk1, a |
| | of cell | wheat skp1 homologue, and functional comparison with |
| | division | arabidopsis ask1 in male meiosis and auxin signalling. Functional |
| | | Plant Biology, 33(4), 381-390. |
| TaSNAC8- | Drought | Mao, H., et al. (2020). “Regulatory changes in TaSNAC8-6A are |
| 6A | tolerance in | associated with drought tolerance in wheat seedlings.” Plant |
| | seedling | Biotechnol J 18(4): 1078-1092. |
| | stage | |
| TaCML36 | Sheath | Lu, L., et al. (2019). “TaCML36, a wheat calmodulin-like protein, |
| | blight | positively participates in an immune response to Rhizoctonia |
| | disease | cerealis.” Crop Journal 7(5): 608-618. |
| | resistance | |
| TdPIP2; 1 | Salt and | Ayadi, M., et al. (2019). “Overexpression of a Wheat Aquaporin |
| | drought | Gene, TdPIP2; 1, Enhances Salt and Drought Tolerance in |
| | tolerance | Transgenic Durum Wheat cv. Maali.” Int J Mol Sci 20(10). |
| TaCIPK10 | Stripe rust | Liu, P., et al. (2019). “TaCIPK10 interacts with and |
| | resistance | phosphorylates TaNH2 to activate wheat defense responses to |
| | | stripe rust.” Plant Biotechnol J 17(5): 956-968. |
| TaCML20 | Drought | Kalaipandian, S., et al. (2019). “Overexpression of TaCML20, a |
| | tolerance | calmodulin-like gene, enhances water soluble carbohydrate |
| | and growth | accumulation and yield in wheat.” Physiol Plant 165(4): 790-799. |
| | promoting | |
| TaJAZ1 | Powdery | Jing, Y., et al. (2019). “Overexpression of TaJAZ1 increases |
| | mildew | powdery mildew resistance through promoting reactive oxygen |
| | resistance | species accumulation in bread wheat.” Sci Rep 9(1): 5691. |
| TaCOLD1 | Reduced | Dong, H., et al. (2019). “TaCOLD1 defines anew regulator of |
| | height of | plant height in bread wheat.” Plant Biotechnol J 17(3): 687-699. |
| | plant | |
| TaMYB86B | Salt and | Song, Y., et al. (2020). “TaMYB86B encodes a R2R3-type MYB |
| | drought | transcription factor and enhances salt tolerance in wheat.” Plant |
| | tolerance | Sci 300: 110624. |
| TaUGT6 | FHB | He, Y., et al. (2020). “TaUGT6, a Novel |
| | resistance | UDP-Glycosyltransferase Gene Enhances the Resistance to FHB |
| | | and DON Accumulation in Wheat.” Front Plant Sci 11: 574775. |
| TaEXPA2 | Drought | Yang, J.J., et al. (2020). “Expansin gene TaEXPA2 positively |
| | tolerance | regulates drought tolerance in transgenic wheat (Triticum |
| | | aestivum L.).” Plant Science 298: 14. |
| TaDof1 | N and C | Hasnain, A., et al. (2020). “Transcription Factor TaDof1 |
| | assimilation | Improves Nitrogen and Carbon Assimilation Under Low-Nitrogen |
| | | Conditions in Wheat.” Plant Molecular Biology Reporter 38(3): |
| | | 441-451. |
| TaPRX-2A | Salt | Su, P., et al. (2020). “A member of wheat class III peroxidase |
| | tolerance | gene family,TaPRX-2A, enhanced the tolerance of salt stress.” |
| | | BMC Plant Biol 20(1). |
| TaPUB1 | Salt | Wang, W., et al. (2020). “The involvement of wheat U-box E3 |
| | tolerance | ubiquitin ligase TaPUB1 in salt stress tolerance.” J Integr Plant |
| | | Biol 62(5): 631-651. |
| TaZFP1B | Drought | Cheuk, A., et al. (2020). “The barley stripe mosaic virus |
| | tolerance | expression system reveals the wheat C2H2 zinc finger protein |
| | | TaZFP1B as a key regulator of drought tolerance.” BMC Plant |
| | | Biol 20(1): 144. |
| TaWAK6 | Leaf rust | Dmochowska-Boguta, M., et al. (2020). “TaWAK6 encoding |
| | resistance | wall-associated kinase is involved in wheat resistance to leaf rust |
| | | similar to adult plant resistance.” PLoS One 15(1): e0227713. |
| TaGATA1 | sheath | Liu, X., et al. (2020). “The wheat LLM-domain-containing |
| | blight | transcription factor TaGATA1 positively modulates host immune |
| | disease | response to Rhizoctonia cerealis.” J Exp Bot 71(1): 344-355. |
| | resistance | |
| TaMAPK16 | Drought | Zhao, Y.J., et al. (2020). “Characterization on the water |
| | tolerance | deprivation-associated physiological traits as well as the related |
| | | differential genes during seed filling stage in wheat (T. aestivum |
| | | L.).” Plant Cell Tissue and Organ Culture 140(3): 605-618. |
| TaPEPKR2 | Heat and | Zang, X., et al. (2018). “Overexpression of the Wheat (Triticum |
| | drought | aestivum L.) TaPEPKR2 Gene Enhances Heat and Dehydration |
| | tolerance | Tolerance in Both Wheat and Arabidopsis.” Front Plant Sci 9: |
| | | 1710. |
| TaWRKY2 | Drought | Gao, H., et al. (2018). “Overexpression of a WRKY Transcription |
| | tolerance | Factor TaWRKY2 Enhances Drought Stress Tolerance in |
| | | Transgenic Wheat.” Front Plant Sci 9: 997. |
| TaNBP1 | Low | Liu, Z., et al. (2018). “TaNBP1, a guanine nucleotide-binding |
| | nitrogen | subunit gene of wheat, is essential in the regulation of N |
| | tolerance | starvation adaptation via modulating N acquisition and ROS |
| | | homeostasis.” BMC Plant Biol 18(1): 167. |
| TaMIR2275 | Low | Qiao, Q.H., et al. (2018). “Wheat miRNA member TaMIR2275 |
| | nitrogen | involves plant nitrogen starvation adaptation via enhancement of |
| | tolerance | the N acquisition-associated process.” Acta Physiologiae |
| | | Plantarum 40(10): 13. |
| TaSHN1 | Drought | Bi, H., et al. (2018). “Overexpression of the TaSHN1 |
| | tolerance | transcription factor in bread wheat leads to leaf surface |
| | | modifications, improved drought tolerance, and no yield penalty |
| | | under controlled growth conditions.” Plant Cell Environ 41(11): |
| | | 2549-2566. |
| TaGS2-2Ab | Promoting | Hu, M., et al. (2018). “Transgenic expression of plastidic |
| | nitrogen | glutamine synthetase increases nitrogen uptake and yield in |
| | use | wheat.” Plant Biotechnol J 16(11): 1858-1867. |
| | efficiency | |
| TaRNAC1 | To enhance | Chen, D., et al. (2018). “Overexpression of a predominantly |
| | drought | root-expressed NAC transcription factor in wheat roots enhances |
| | tolerance | root length, biomass and drought tolerance.” Plant Cell Rep |
| | of root | 37(2): 225-237. |
| | system | |
| TaEDS1 | Powdery | Chen, G., et al. (2018). “TaEDS1 genes positively regulate |
| | mildew | resistance to powdery mildew in wheat.” Plant Mol Biol 96(6): |
| | resistance | 607-625. |
| Ta-UGT (3) | Head blight | Xing, L.P., et al. (2018). “Over-expressing a |
| | resistance | UDP-glucosyltransferase gene (Ta-UGT (3)) enhances Fusarium |
| | | Head Blight resistance of wheat.” Plant Growth Regulation 84(3): |
| | | 561-571. |
| aCOMT-3D | Sheath | Wang, M., et al. (2018). “A wheat caffeic acid |
| | blight | 3-O-methyltransferase TaCOMT-3D positively contributes to |
| | disease | both resistance to sharp eyespot disease and stem mechanical |
| | resistance | strength.” Sci Rep 8(1): 6543. |
| TaCIPK23 | Drought | Cui, X.Y., et al. (2018). “Wheat CBL-interacting protein kinase |
| | tolerance | 23 positively regulates drought stress and ABA responses.” BMC |
| | | Plant Biol 18(1): 93. |
| TaSAP5 | Drought | Zhang, N., et al. (2017). “The E3 Ligase TaSAP5 Alters Drought |
| | tolerance | Stress Responses by Promoting the Degradation of DRIP |
| | | Proteins.” Plant Physiol 175(4): 1878-1892. |
| TaTAR2.1- | To increase | Shao, A., et al. (2017). “The Auxin Biosynthetic TRYPTOPHAN |
| 3A | yield and | AMINOTRANSFERASE RELATED TaTAR2.1-3A Increases |
| | biomass | Grain Yield of Wheat.” Plant Physiol 174(4): 2274-2288. |
| TaRCR1 | Sheath | Zhu, X., et al. (2017). “The wheat NB-LRR gene TaRCR1 is |
| | blight | required for host defence response to the necrotrophic fungal |
| | disease | pathogen Rhizoctonia cerealis.” Plant Biotechnol J 15(6): |
| | resistance | 674-687. |
| TaPIMP2 | root rot | Wei, X., et al. (2017). “TaPIMP2, a pathogen-induced MYB |
| | resistance | protein in wheat, contributes to host resistance to common root |
| | | rot caused by Bipolaris sorokiniana.” Sci Rep 7(1): 1754. |
| TaNF-YB3; 1 | Drought | Yang, M.Y., et al. (2017). “Wheat nuclear factor Y (NF-Y) B |
| | tolerance | subfamily gene TaNF-YB3; 1 confers critical drought tolerance |
| | | through modulation of the ABA-associated signaling pathway.” |
| | | Plant Cell Tissue and Organ Culture 128(1): 97-111. |
| TaFER-5B | Heat and | Zang, X., et al. (2017). “Overexpression of wheat ferritin gene |
| | other | TaFER-5B enhances tolerance to heat stress and other abiotic |
| | tolerance | stresses associated with the ROS scavenging.” BMC Plant Biol |
| | | 17(1): 14. |
| TaCAD12 | Sheath | Rong, W., et al. (2016). “A Wheat Cinnamyl Alcohol |
| | blight | Dehydrogenase TaCAD12 Contributes to Host Resistance to the |
| | disease | Sharp Eyespot Disease.” Front Plant Sci 7: 1723. |
| | resistance | |
| TaCPK7-D | Sheath | Wei, X., et al. (2016). “The wheat calcium-dependent protein |
| | blight | kinase TaCPK7-D positively regulates host resistance to sharp |
| | disease | eyespot disease.” Mol Plant Pathol 17(8): 1252-1264. |
| | resistance | |
| TabHLH1 | Nitrogen | Yang, T., et al. (2016). “TabHLH1, a bHLH-type transcription |
| | and | factor gene in wheat, improves plant tolerance to Pi and N |
| | phosphorus | deprivation via regulation of nutrient transporter gene |
| | stress | transcription and ROS homeostasis.” Plant Physiol Biochem 104: |
| | tolerance | 99-113. |
| TaRIM1 | Sheath | Shan, T., et al. (2016). “The wheat R2R3-MYB transcription |
| | blight | factor TaRIM1 participates in resistance response against the |
| | disease | pathogen Rhizoctonia cerealis infection through regulating |
| | resistance | defense genes.” Sci Rep 6: 28777. |
| TaBASS2 | Salt | Zhao, Y., et al. (2016). “A putative pyruvate transporter |
| | tolerance | TaBASS2 positively regulates salinity tolerance in wheat via |
| | | modulation of ABI4 expression.” BMC Plant Biol 16(1): 109. |
| TaSOD2 | Salt stress | Wang, M., et al. (2016). “A wheat superoxide dismutase gene |
| | and other | TaSOD2 enhances salt resistance through modulating redox |
| | stresses | homeostasis by promoting NADPH oxidase activity.” Plant Mol |
| | | Biol 91(1-2): 115-130. |
| TaRLK1/ | Powdery | Chen, T., et al. (2016). “Two members of TaRLK family confer |
| TaRLK2 | mildew | powdery mildew resistance in common wheat.” BMC Plant Biol |
| | resistance | 16: 27. |
| Wknox1 | Formation | Ryoko Morimoto, et al. (2005)Intragenic diversity and functional |
| | of leaf | conservation of the three homoeologous loci of the KN1-type |
| | blade | homeobox gene Wknox1 in common wheat. Plant Molecular |
| | | Biology, 57(6). |
| FKBP | Stress | Kurek L, et al. (2002) Overexpression of the wheat fk506-binding |
| | response | protein 73 (fkbp73) and the heat-induced wheat fkbp77 in |
| | and | transgenic wheat reveals different functions of the two isoforms. |
| | photosynthesis | Transgenic Res, 11(4): 373-9. |
| | enhancing | |
| TaMloA/B/D | Powdery | Elliott C., et al. (2002) Functional conservation of wheat and rice |
| | mildew | mio orthologs in defense modulation to the powdery mildew |
| | resistance | fungus. Mol Plant Microbe Interact, 15(10): 1069-77. |
| GLP | Disease | Christensen A.B., et al.(2004), The germinlike protein glp4 |
| | resistance | exhibits superoxide dismutase activity and is an important |
| | | component of quantitative resistance in wheat and barley [J]. Mol |
| | | Plant Microbe Interact, 17(1): 109-17. |
| Q gene | wide | Simons K.J., et al. (2006), Molecular characterization of the |
| | adaptability, | major wheat domestication gene q. Genetics, 172(1): 547-55. |
| | plant | |
| | morphology | |
| TaGI1 | Flowering | Zhao X.Y., et al. (2005), The wheat tagi1, involved in |
| | time | photoperiodic flowering, encodes an arabidopsis gi ortholog[J]. |
| | regulation | Plant Mol Biol, 58(1): 53-64.. |
| TaWRKY45 | Head blight | Bahrini, L, et al. (2011). “Overexpression of the |
| | resistance | pathogen-inducible wheat TaWRKY45 gene confers disease |
| | | resistance to multiple fungi in transgenic wheat plants.” Breed Sci |
| | | 61(4): 319-326. |
| Rht-A1 | Regulation | Pearce, S., et al. (2011). “Molecular characterization of Rht-1 |
| | of plant | dwarfing genes in hexapioid wheat.” Plant Physiol 157(4): |
| | height | 1820-1831. |
| TaSnRK2.7 | Abio-stress | Zhang, H., et al. (2011). “Characterization of a common wheat |
| | tolerance | (Triticum aestivum L.) TaSnRK2.7 gene involved in abiotic stress |
| | | responses.” J Exp Bot 62(3): 975-988. |
| BLF | Head blight | Han, J., et al. (2012). “Transgenic expression of lactoferrin |
| | resistance | imparts enhanced resistance to head blight of wheat caused by |
| | | Fusarium graminearum.” BMC Plant Biol 12: 33. |
| HvCO9 | Flowering | Kikuchi, R., et al. (2012). “The differential expression of HvCO9, |
| | time | a member of the CONSTANS-like gene family, contributes to the |
| | regulation | control of flowering under short-day conditions in barley.” J Exp |
| | | Bot 63(2): 773-784. |
| DELLA | Disease | Saville, R. J., et al. (2012). “The ‘Green Revolution’ dwarfing |
| | resistance | genes play a role in disease resistance in Triticum aestivum and |
| | | Hordeum vulgare.” J Exp Bot 63(3): 1271-1283. |
| TaBI-1 | Stripe rust | Wang, X., et al. (2012). “Wheat BAX inhibitor-1 contributes to |
| | resistance | wheat resistance to Puccinia striiformis.” J Exp Bot 63(12): |
| | | 4571-4584. |
| TaGAMYB | Anther | Wang, Y., et al. (2012). “TamiR159 directed wheat TaGAMYB |
| | development | cleavage and its involvement in anther development and heat |
| | and heat | response.” PLoS One 7(11): e48445. |
| | response | |
| TaMYB32 | Salt | Zhang, L., et al. (2012). “Molecular characterization of 60 |
| | tolerance | isolated wheat MYB genes and analysis of their expression during |
| | | abiotic stress.” J Exp Bot 63(1): 203-214. |
| TaOPR1 | Salt | Dong, W., et al. (2013). “Wheat oxophytodienoate reductase gene |
| | tolerance | TaOPR1 confers salinity tolerance via enhancement of abscisic |
| | | acid signaling and reactive oxygen species scavenging.” Plant |
| | | Physiol 161(3): 1217-1228. |
| FgLaeA | Fusarium | Kim, H.K., et al. (2013). “Functional roles of FgLaeA in |
| | graminearum | controlling secondary metabolism, sexual development, and |
| | resistance | virulence in Fusarium graminearum.” PLoS One 8(7): e68441. |
| TiMYB2R-1 | full rot | Liu, X., et al. (2013). “Transgenic wheat expressing Thinopyrum |
| | disease | intermedium MYB transcription factor TiMYB2R-1 shows |
| | | enhanced resistance to the take-all disease.” J Exp Bot 64(8): |
| | | 2243-2253. |
| FgStuA | Fusarium | Pasquali, M., et al. (2013). “FeStuA from Fusarium culmorum |
| | culmorum | controls wheat foot and root rot in a toxin dispensable manner.” |
| | | PLoS One 8(2): e57429. |
| TaWRKY71-1 | Dormancy | Qin, Z., et al. (2013). “Ectopic expression of a wheat WRKY |
| | regulation | transcription factor gene TaWRKY71-1 results in hyponastic |
| | | leaves in Arabidopsis thaliana.″ PLoS One 8(5): e63033. |
| AbaA | Fusarium | Son, H., et al. (2013). ″AbaA regulates conidiogenesis in the |
| | graminearum | ascomycete fungus Fusarium graminearum.” PLoS One 8(9): |
| | resistance | e72915. |
| ZCCT | Flowering | Gulyas, Z., et al. (2014). “Central role of the flowering repressor |
| | regulation | ZCCT2 in the redox control of freezing tolerance and the initial |
| | | development of flower primordia in wheat.” BMC Plant Biol 14: |
| | | 91. |
| Ta-sro1 | Abio-stress | Liu, S., et al. (2014). “A wheat SIMILAR TO RCD-ONE gene |
| | tolerance | enhances seedling growth and abiotic stress resistance by |
| | | modulating redox homeostasis and maintaining genomic |
| | | integrity.” Plant Cell 26(1): 164-180. |
| TaTEF-7A | Yield | Zheng, J., et al. (2014). “TEF-7A, a transcript elongation factor |
| | | gene, influences yield-related traits in bread wheat (Triticum |
| | | aestivum L.).” J Exp Bot 65(18): 5351-5365. |
| TaNAC2-5A | Yield | He, X., et al. (2015). “The Nitrate-Inducible NAC Transcription |
| | | Factor TaNAC2-5A Controls Nitrate Response and Increases |
| | | Wheat Yield.” Plant Physiol 169(3): 1991-2005. |
| TaFROG | Fusarium | Perochon, A., et al. (2015). “TaFROG Encodes a Pooideae |
| | graminearum | Orphan Protein That Interacts with SnRK1 and Enhances |
| | | Resistance to the Mycotoxigenic Fungus Fusarium graminearum.” |
| | resistance | Plant Physiol 169(4): 2895-2906. |
| PsANT | Disease | Tang, C., et al. (2015). “PsANT, the adenine nucleotide |
| | resistance | translocase of Puccinia striiformis, promotes cell death and |
| | | fungal growth.” Sci Rep 5: 11241. |
| SbPIP1 | Salt | Yu, G.H., et al. (2015). “Changes in the Physiological |
| | tolerance | Parameters of SbPIP1-Transformed Wheat Plants under Salt |
| | | Stress.” Int J Genomics 2015: 384356. |
| TaNAC47 | Abio-stress | Zhang, L., et al. (2015). “The Novel Wheat Transcription Factor |
| | tolerance | TaNAC47 Enhances Multiple Abiotic Stress Tolerances in |
| | | Transgenic Plants.” Front Plant Sci 6: 1174. |
| TaFBA1 | Anti-oxidation | Zhou, S.M., et al. (2015). “The involvement of wheat F-box |
| | | protein gene TaFBA1 in the oxidative stress tolerance of plants.” |
| | | PLoS One 10(4): e0122117. |
| CMPG1-V | Powdery | Zhu, Y., et al. (2015). “E3 ubiquitin ligase gene CMPG1-V from |
| | mildew | Haynaldia villosa L. contributes to powdery mildew resistance in |
| | resistance | common wheat (Triticum aestivum L.).” Plant J 84(1): 154-168. |
| TaAGC1 | Rhizoctonia | Zhu, X., et al. (2015). “The wheat AGC kinase TaAGC1 is a |
| | cerealis | positive contributor to host resistance to the necrotrophic |
| | resistance | pathogen Rhizoctonia cerealis.” J Exp Bot 66(21): 6591-6603. |
| TaNAC-S | Late | Zhao, D., et al. (2015). “Overexpression of aNAC transcription |
| | maturing, | factor delays leaf senescence and increases grain nitrogen |
| | improve | concentration in wheat.” Plant Biol (Stuttg) 17(4): 904-913. |
| | grain seed | |
| | quality | |
| TabZIP60 | Abio-stress | Zhang, L., et al. (2015). “A novel wheat bZIP transcription factor, |
| | tolerance | TabZIP60, confers multiple abiotic stress tolerances in transgenic |
| | | Arabidopsis.” Physiol Plant 153(4): 538-554. |
| TaNF-YB4 | Promoting | Yadav, D., et al. (2015). “Constitutive overexpression of the |
| | yield | TaNF-YB4 gene in transgenic wheat significantly improves grain |
| | | yield.” J Exp Bot 66(21): 6635-6650. |
| TaHsfA6f | Heat | Xue, G.P., et al. (2015). “TaHsfA6f is a transcriptional activator |
| | response | that regulates a suite of heat stress protection genes in wheat |
| | | (Triticum aestivum L.) including previously unknown Hsf |
| | | targets.” J Exp Bot 66(3): 1025-1039. |
| TaNAC29 | Salt | Xu, Z., et al. (2015). “Wheat NAC transcription factor TaNAC29 |
| | tolerance | is involved in response to salt stress.” Plant Physiol Biochem 96: |
| | | 356-363. |
| CYP51 | Aluminum | Wagatsuma, T., et al. (2015). “Higher sterol content regulated by |
| | tolerance | CYP51 with concomitant lower phospholipid content in |
| | | membranes is a common strategy for aluminium tolerance in |
| | | several plant species.” J Exp Bot 66(3): 907-918. |
| TaGBF1 | Blue light | Sun, Y., et al. (2015). “The wheat TaGBF1 gene is involved in the |
| | response | blue-light response and salt tolerance.” Plant J 84(6): 1219-1230. |
| | and salt | |
| | tolerance | |
| EF-Tu | Disease | Schoonbeek, H.J., et al. (2015). “Arabidopsis EF-Tu receptor |
| | resistance | enhances bacterial disease resistance in transgenic wheat.” New |
| | | Phytol 206(2): 606-613. |
| Chs3b | Disease | Cheng, W., et al. (2015). “Host-induced gene silencing of an |
| | resistance | essential chitin synthase gene confers durable resistance to |
| | | Fusarium head blight and seedling blight in wheat.” Plant |
| | | Biotechnol J 13(9): 1335-1345. |
| PsSRPKL | Fusarium | Cheng, Y., et al. (2015). “Characterization of protein kinase |
| | head blight | PsSRPKL, a novel pathogenicity factor in the wheat stripe rust |
| | resistance | fungus.” Environ Microbiol 17(8): 2601-2617. |
| TdSHN1 | Abio-stress | Djemal, R. and H. Khoudi (2015). “Isolation and molecular |
| | tolerance | characterization of a novel WIN1/SHN1 ethylene-responsive |
| | | transcription factor TdSHN1 from durum wheat (Triticum |
| | | turgidum. L. subsp. durum).” Protoplasma 252(6): 1461-1473. |
| WD40 | Abio-stress | Kong, D., et al. (2015). “Identification of TaWD40D, a wheat |
| | tolerance | WD40 repeat-containing protein that is associated with plant |
| | | tolerance to abiotic stresses.” Plant Cell Rep 34(3): 395-410. |
| TaCYP78A3 | Grain size | Ma, M., et al. (2015). “Expression of TaCYP78A3, a gene |
| | regulation | encoding cytochrome P450 CYP78A3 protein in wheat (Triticum |
| | | aestivum L.), affects seed size.” Plant J 83(2): 312-325. |
| TaDOGIL4 | Seed | Ashikawa, I., et al. (2014). “A transgenic approach to controlling |
| | dormancy | wheat seed dormancy level by using Triticeae DOG1-like genes.” |
| | regulation | Transgenic Res 23(4): 621-629. |
| TaEXPB23 | Promoting | Han, Y.Y., et al. (2014). “The involvement of expansins in |
| | uptake of | responses to phosphorus availability in wheat, and its potentials |
| | phosphorus | in improving phosphorus efficiency of plants.” Plant Physiol |
| | | Biochem 78: 53-62. |
| TaNHX3 | Salt | Lu, W., et al. (2014). “Overexpression of TaNHX3, a vacuolar |
| | tolerance | Na(+)/H(+) antiporter gene in wheat, enhances salt stress |
| | | tolerance in tobacco by improving related physiological |
| | | processes.” Plant Physiol Biochem 76: 17-28. |
| TaERF3 | Abio-stress | Rong, W., et al. (2014). “The ERF transcription factor TaERF3 |
| | tolerance | promotes tolerance to salt and drought stresses in wheat.” Plant |
| | | Biotechnol J 12(4): 468-479. |
| TaABL1 | Abio-stress | Xu, D.B., et al. (2014). “ABI-like transcription factor gene |
| | tolerance | TaABL1 from wheat improves multiple abiotic stress tolerances |
| | | in transgenic plants.” Funct Integr Genomics 14(4): 717-730. |
| TaHsfC2a | Thermo | Xue, G.P., et al. (2014). “The heat shock factor family from |
| | tolerance | Triticum aestivum in response to heat and other major abiotic |
| | | stresses and their role in regulation of heat shock protein genes.” |
| | | J Exp Bot 65(2): 539-557. |
| TaLTP | Chilling | Yu, G., et al. (2014). “Identification of wheat non-specific lipid |
| | tolerance | transfer proteins involved in chilling tolerance.” Plant Cell Rep |
| | | 33(10): 1757-1766. |
| TaCLP1 | Stripe rust | Feng, H., et al. (2013). “Target of tae-miR408, a |
| | resistance | chemocyanin-like protein gene (TaCLP1), plays positive roles in |
| | | wheat response to high-salinity, heavy cupric stress and stripe |
| | | rust.” Plant Mol Biol 83(4-5): 433-443. |
| TaLSD1 | Stripe rust | Guo, J., et al. (2013). “Wheat zinc finger protein TaLSD1, a |
| | resistance | negative regulator of programmed cell death, is involved in wheat |
| | | resistance against stripe rust fungus.” Plant Physiol Biochem 71: |
| | | 164-172. |
| TaLSU1 | Starch | Kang, G., et al. (2013). “Increasing the starch content and grain |
| | content | weight of common wheat by overexpression of the cytosolic |
| | | AGPase large subunit gene.” Plant Physiol Biochem 73: 93-98. |
| TaDREB3 | Chilling | Kovalchuk, N., et al. (2013). “Optimization of TaDREB3 gene |
| | tolerance | expression in transgenic barley using cold-inducible promoters.” |
| | | Plant Biotechnol J 11(6): 659-670. |
| TaS3 | Powdery | Li, S., et al. (2013). “Wheat gene TaS3 contributes to powdery |
| | mildew | mildew susceptibility.” Plant Cell Rep 32(12): 1891-1901. |
| TaHMA2 | Heavy-metal | Tan, J., et al. (2013). “Functional analyses of TaHMA2, a |
| | tolerance | P(1B)-type ATPase in wheat.” Plant Biotechnol J 11(4): 420-431. |
| TaSnRK2.3 | Abio-stress | Tian, S., et al. (2013). “Cloning and characterization of |
| | tolerance | TaSnRK2.3, a novel SnRK2 gene in common wheat.” J Exp Bot |
| | | 64(7): 2063-2080. |
| TaMYB3R1 | Abio-stress | Cai, H., et al. (2011). “Identification of a MYB3R gene involved |
| | tolerance | in drought, salt and cold stress in wheat (Triticum aestivum L.).” |
| | | Gene 485(2): 146-152. |
| TaLTP5 | Fusarium | Zhu, X., et al. (2012). “Overexpression of wheat lipid transfer |
| | graminearum | protein gene TaLTP5 increases resistances to Cochliobolus |
| | resistance | sativus and Fusarium graminearum in transgenic wheat.” Funct |
| | | Integr Genomics 12(3): 481-488. |
| TaCHP | Salt | Zhao, X., et al. (2012). “The role of TaCHP in salt stress |
| | resistance | responsive pathways.” Plant Signal Behav 7(1): 71-74. |
| TaDAD2 | Stripe rust | Wang, X., et al. (2011). “TaDAD2, a negative regulator of |
| | resistance | programmed cell death, is important for the interaction between |
| | | wheat and the stripe rust fungus.” Mol Plant Microbe Interact |
| | | 24(1): 79-90. |

Table J lists some representative functional genes in tomato.

TABLE J
Important functional genes in in tomato

| Gene name | Application | Reference |
|---|---|---|
| SlWRKY3 | SlWRKY3 as a positive | Chinnapandi, B., et al. (2019). “Tomato |
| | regulator of induced | SlWRKY3 acts as a positive regulator for |
| | resistance in response | resistance against the root-knot nematode |
| | to nematode invasion | Meloidogyne javanica by activating lipids and |
| | and infection, mostly | hormone-mediated defense-signaling pathways.” |
| | during the early stages | Plant Signal Behav 14(6): 1601951. |
| | of nematode infection. | |
| SlSAMS1 | tolerance to alkali | Gong, B., et al. (2014). “Overexpression of |
| | stress | S-adenosyl-L-methionine synthetase increased |
| | | tomato tolerance to alkali stress through |
| | | polyamine metabolism.” Plant Biotechnol J |
| | | 12(6): 694-708. |
| SlCYP90B3 | BR biosynthesis | Hu, S., et al. (2020). “Regulation of fruit |
| | | ripening by the brassinosteroid biosynthetic gene |
| | | SlCYP90B3 via an ethylene-dependent pathway |
| | | in tomato.” Hortic Res 7: 163. |
| SlTLFP8 | decreased stomatai | Li, S., et al. (2020). “SlTLFP8 reduces water loss |
| | density | to improve water-use efficiency by modulating |
| | | cell size and stomatal density via |
| | | endoreduplication.” Plant Cell Environ 43(11): |
| | | 2666-2679. |
| DWARF | improved seed | Li, X.J., et al. (2016). “DWARF overexpression |
| | germination, root | induces alteration in phytohormone homeostasis, |
| | development and early | development, architecture and carotenoid |
| | growth vigour | accumulation in tomato.” Plant Biotechnol J |
| | | 14(3): 1021-1033. |
| SlAGO7 | increased fruit yield | Lin, D., et al. (2016). “Ectopic expression of |
| | | SlAGO7 alters leaf pattern and inflorescence |
| | | architecture and increases fruit yield in tomato.” |
| | | Physiol Plant 157(4): 490-506. |
| LeNHX4 | increased fruit size | Maach, M., et al. (2020). “Overexpression of |
| | | LeNHX4 improved yield, fruit quality and salt |
| | | tolerance in tomato plants |
| | | (Solanumlycopersicum L.).” MolBiol Rep 47(6): |
| | | 4145-4153. |
| SlBRI1 | improve multiple major | Nie, S., et al. (2017). “Enhancing |
| | agronomic traits | Brassinosteroid Signaling via Overexpression of |
| | | Tomato (Solanumlycopersicum) SlBRI1 |
| | | Improves Major Agronomic Traits.” Front Plant |
| | | Sci 8: 1386. |
| SlCDF4 | increased yield | Renau-Morata, B., et al. (2020). “The targeted |
| | | overexpression of SlCDF4 in the fruit enhances |
| | | tomato size and yield involving gibberellin |
| | | signalling.” Sci Rep 10(1): 10645. |
| SlJUB1 | drought tolerance | Thirumalaikumar, V.P., et al. (2018). “NAC |
| | | transcription factor JUNGBRUNNEN1 enhances |
| | | drought tolerance in tomato.” Plant Biotechnol J |
| | | 16(2): 354-366. |
| SlNAP1 | drought tolerance | Wang, J., et al. (2020). “Transcriptomic and |
| | | genetic approaches reveal an essential role of the |
| | | NAC transcription factor SlNAP1 in the growth |
| | | and defense response of tomato.” Hortic Res |
| | | 7(1): 209. |
| SlRING1 | cadmium (Cd) | Ahammed, G.J., et al. (2020). “Overexpression |
| | tolerance | of tomato RING E3 ubiquitin ligase gene |
| | | SlRING1 confers cadmium tolerance by |
| | | attenuating cadmium accumulation and oxidative |
| | | stress.” Physiol Plant. |
| SlAREB1 | regulates primary | Bastias, A., et al. (2014). “The transcription |
| | metabolic pathways | factor AREB1 regulates primary metabolic |
| | | pathways in tomato fruits.” J Exp Bot 65(9): |
| | | 2351-2363. |
| HsfA1a | cadmium (Cd) | Cai, S.Y., et al. (2017). “HsfA1a upregulates |
| | tolerance | melatonin biosynthesis to confer cadmium |
| | | tolerance in tomato plants.” J Pineal Res 62(2) |
| SlUPA-like | regulation of plant | Cui, B., et al. (2016). “Overexpression of |
| | development and stress | SlUPA-like induces cell enlargement, aberrant |
| | tolerance | development and low stress tolerance through |
| | | phytohormonal pathway in tomato.” Sci Rep 6: |
| | | 23818 |
| MYB49 | tolerance to drought | Cui, J., et al. (2018). “Tomato MYB49 enhances |
| | and salt stresses | resistance to Phytophthorainfestans and |
| | | tolerance to water deficit and salt stress.” Planta |
| | | 248(6): 1487-1503. |
| LetAPX | tolerance to chilling | Duan, M., et al. (2012). “Overexpression of |
| | stress | thylakoidalascorbate peroxidase shows enhanced |
| | | resistance to chilling stress in tomato.” J Plant |
| | | Physiol 169(9): 867-877. |
| SlBZR1D | regulates BR signaling | Jia, C., et al. (2021). “Tomato BZR/BES |
| | and salt tolerance | transcription factor SlBZR1 positively regulates |
| | | BR signaling and salt stress tolerance in tomato |
| | | and Arabidopsis.” Plant Sci 302: 110719. |
| SlMBP22 | drought tolerance | Li, F., et al. (2020). “Overexpression of |
| | | SlMBP22 in Tomato Affects Plant Growth and |
| | | Enhances Tolerance to Drought Stress.” Plant |
| | | Sci 301: 110672. |
| SlCOMT1 | salt tolerance | Liu, D.D., et al. (2019). “Overexpression of the |
| | | Melatonin Synthesis-Related Gene SlCOMT1 |
| | | Improves the Resistance of Tomato to Salt |
| | | Stress.” Molecules 24(8). |
| SlGRAS40 | enhances tolerance to | Liu, Y., et al. (2017). “Overexpression of |
| | Abiotic Stresses and | SlGRAS40 in Tomato Enhances Tolerance to |
| | influences Auxin and | Abiotic Stresses and Influences Auxin and |
| | Gibberellin signaling | Gibberellin Signaling.” Front Plant Sci 8: 1659. |
| SlGMEs | increased ascorbate | Zhang, C., et al. (2011). “Overexpression of |
| | accumulation and | SIGMEs leads to ascorbate accumulation with |
| | improved tolerance to | enhanced oxidative stress, cold, and salt |
| | abiotic stresses | tolerance in tomato.” Plant Cell Rep 30(3): |
| | | 389-398. |
| SlMAPK3 | regulates tolerance to | Muhammad, T., et al. (2019). “Overexpression |
| | Cd(2+) and drought | of a Mitogen-Activated Protein Kinase |
| | stress | SlMAPK3 Positively Regulates Tomato |
| | | Tolerance to Cadmium and Drought Stress.” |
| | | Molecules 24(3). |

Table K lists some representative functional genes in potato and sweet potato.

TABLE K
Important functional genes in potato and sweetpotato

| Crop | Gene name | Application | Reference |
|---|---|---|---|
| potato | StERF94 | Salt | Charfeddine M, et al.(2019). “Investigation of |
| | | tolerance | the response to salinity of transgenic potato |
| | | | plants overexpressing the transcription factor |
| | | | StERF94”. Journal of Biosciences, 44(6). |
| potato | STARCH | To reduce | Brummell D A, et al. (2015). “Overexpression |
| | BRANCHING | gelatinization | of STARCH BRANCHING ENZYME II |
| | ENZYME | temperature, | increases short-chain branching of |
| | II(SBEII) | to change | amylopectin and alters the physicochemical |
| | | starch | properties of starch from potato tuber”. BMC |
| | | properties | Biotechnology. |
| potato | potato protease | Reduced | Dong T, et al. (2020). “Cysteine protease |
| | inhibitors (StPIs) | enzymatic | inhibitors reduce enzymatic browning of |
| | | browning | potato by lowering the accumulation of free |
| | | | amino acids”. Journal of Agricultural and Food |
| | | | Chemistry. |
| potato | NAC family | Wilt | Chang Y, et al. (2020). “NAC transcription |
| | transcription | resistance in | factor involves in regulating bacterial wilt |
| | factor (StNACb4) | potato | resistance in potato”. Functional Plant |
| | | | Biology, 47. |
| potato | nitrate transporter | Increased | KLAASSEN M T, et al. (2020). |
| | gene(StNPF1.11) | nitrogen use | “Overexpression of a putative nitrate |
| | | efficiency, | transporter (StNPF1.11) increases plant height, |
| | | plant height, | leaf chlorophyll content and tuber protein |
| | | leaf | content of young potato plants”. Funct Plant |
| | | chlorophyll | Biol, 47(5): 464-472 |
| | | content and | |
| | | tuber protein | |
| | | content | |
| potato | eukaryotic | PVY | Sanchez P A G, et al. (2020). “Overexpression |
| | translation | resistance | of a modified eIF4E regulates potato virus Y |
| | initiation factor 4E | | resistance at the transcriptional level in |
| | (eIF4E) | | potato”. BMC Genomics, 21. |
| potato | StPOTHR1 | Late blight | Chen Q, et al. (2018). “StPOTHR1, a |
| | | resistance | NDR1/HIN1-like gene in Solanumtuberosum, |
| | | | enhances resistance against |
| | | | Phytophthorainfestans”. Biochemical & |
| | | | Biophysical Research |
| | | | Communications,1 155-1161. |
| potato | Snakin-1 (SN1) | Enhance | NATALIA, et al. (2008). “Overexpression of |
| | | resistance to | snakin-1 gene enhances resistance to |
| | | bacterial | Rhizoctoniasolani and Erwiniacarotovora in |
| | | disease | transgenic potato plants”. Molecular Plant |
| | | | Pathology, 9(3):329-338. |
| potato | Phosphate | Growth | Cao M, et al. (2020). “Functional Analysis of |
| | Transporter | promoting | StPHT1; 7, a Solanumtuberosum L. Phosphate |
| | PHT1; 7 | | Transporter Gene, in Growth and Drought |
| | | | Tolerance”. Plants, 9(10): 1384. |
| potato | StBUS/ELS | Tolerance to | Varun Dwivedi, et al. (2020). “Functional |
| | | certain | characterization of a defense-responsive |
| | | bacteria and | bulnesol/elemol synthase from potato”. |
| | | fungi | PhysiologiaPlantarum. |
| potato | StDREB1 | Increase | Donia Bouaziz, et al. (2013). “Overexpression |
| | | tolerance to | of StDREB1 Transcription Factor Increases |
| | | salt | Tolerance to Salt in Transgenic Potato Plants”. |
| | | | Molecular Biotechnology, 54(3):803-817 |
| potato | StMAPK11 | To promote | Zhu X, et al. (2021). “Mitogen-activated |
| | | growth under | protein kinase 11 (MAPK11) maintains growth |
| | | drought | and photosynthesis of potato plant under |
| | | condition | drought condition”. Plant Cell Reports,1-16. |
| potato | POTH1 | Enlarged | Rosin F M, et al. (2003). “Overexpression of a |
| | | tube | Knotted-like Homeobox Gene of Potato Alters |
| | | | Vegetative Development by Decreasing |
| | | | Gibberellin Accumulation”. Plant Physiology, |
| | | | 132(1): 106-117. |
| potato | StMPKl | Late blight | Yamamizo C, et al. (2006). “Rewiring |
| | | resistance | Mitogen-Activated Protein Kinase Cascade by |
| | | | Positive Feedback Confers Potato Blight |
| | | | Resistance”. Plant Physiology, 140(2):681-692 |
| potato | S. | Cold and salt | Lee H E, et al. (2007). “Ethylene responsive |
| | tuberosumethylene | stress | element binding protein 1 (StEREBP1) from |
| | responsive | tolerance | Solanumtuberosum increases tolerance to |
| | element binding | | abiotic stress in transgenic potato plants”. |
| | protein | | Biochemical & Biophysical Research |
| | (StEREBP1) | | Communications, 353(4): 863-868. |
| potato | StBEL5 | Enlarged | Mithu Chatterjee, et al, (2007). “A |
| | | tube | BELL1-Like Gene of Potato Is Light Activated |
| | | | and Wound Inducible”. Plant Physiology, |
| | | | Volume 145, Issue 4, 1435-1443, |
| potato | StRFP1 | Broad | Ni X, et al. (2010). “Cloning and molecular |
| | | spectrum | characterization of the potato RING finger |
| | | resistance to | protein gene StRFP1 and its function in potato |
| | | late blight | broad-spectrum resistance against |
| | | | Phytophthorainfestans”. Journal of Plant |
| | | | Physiology, 167(6):488-496. |
| potato | StMYB1R-1 | Drought | Shin D, et al. (2011). “Expression of |
| | | tolerance | StMYB1R-1, a novel potato single MYB-like |
| | | | domain transcription factor, increases drought |
| | | | tolerance”. Plant Physiology, 155(l):421-432. |
| potato | StInvInh2A | Reduced | Liu X, et al. (2013). “StInvInh2 as an inhibitor |
| | StInvInh2B | cold-induced | of StvacINV1 regulates the cold-induced |
| | | sweetening | sweetening of potato tubers by specifically |
| | | of potato | capping vacuolar invertase activity”. Plant |
| | | | Biotechnology Journal, 11(5):640-647. |
| potato | StAN11 | Increased | Li W, et al. (2013). “Cloning and |
| | | anthocyanin | characterization of a potato StAN11 gene |
| | | accumulation | involved in anthocyanin biosynthesis |
| | | | regulation”. Journal of Integrative Plant |
| | | | Biology, 56(4):364-372. |
| potato | STANN1 | Drought | Michal, et al. (2015). “Potato Annexin |
| | | tolerance | STANN1 Promotes Drought Tolerance and |
| | | | Mitigates Light Stress in Transgenic |
| | | | Solanumtuberosum L. Plants”. Plos One. |
| sweet | IbOr | Increased | Goo Y M, et al. (2015). “Overexpression of |
| potato | | accumulation | the sweet potato IbOr gene results in the |
| | | of carotenoid | increased accumulation of carotenoid and |
| | | and confers | confers tolerance to environmental stresses in |
| | | tolerance to | transgenic potato”. ComptesRendusBiologies. |
| | | salt stress | |
| sweet | IbPSS1 | Increased | Yu Y, et al. (2020). “Overexpression of |
| potato | | salt tolerance | phosphatidylserine synthase IbPSS1 affords |
| | | in root | cellular Na+ homeostasis and salt tolerance by |
| | | | activating plasma membrane Na+/H+ antiport |
| | | | activity in sweet potato roots”. Horticulture |
| | | | Research, 7:131. |
| sweet | IbLCY-e | Increased | Kim S H, et al. (2013). “Downregulation of the |
| potato | | salt tolerance | lycopene-cyclase gene increases carotenoid |
| | | | synthesis via the P-branch-specific pathway |
| | | | and enhances salt-stress tolerance in sweet |
| | | | potato transgenic calli”. Physiologia |
| | | | Plantarum, 147(4):432-442. |
| sweet | IbNFU1 | Increased | Liu, D.G., et al (2014b) |
| potato | | salt tolerance | Anlpomoeabatatasiron-sulfur cluster scaffold |
| | | | protein gene, IbNFU1, is involved in salt |
| | | | tolerance. PLoS One, 9, e93935 |
| sweet | IbP5CR | Increased | Liu, D.G. et al (2014a) Overexpression |
| potato | | salt tolerance | of IbP5CR enhances salt tolerance in |
| | | | transgenie sweet potato. Plant Cell, Tissue |
| | | | Organ Cult. 117(1),1-16 |
| sweet | IbMas | Increased | Liu, D.G., et al (2014c) A novela/b-hydrolase |
| potato | | salt tolerance | gene IbMas enhances salt tolerance in |
| | | | transgenic sweetpotato PLoS One, 9, e115128. |
| sweet | IbSIMT1 | Increased | Liu, D.G., et al (2015) IbSIMT1, a novel |
| potato | | salt tolerance | salt-induced methyltransferase gene from |
| | | | Ipomoea batatas, is involved in salt |
| | | | tolerance .Plant Cell, Tissue Organ Cult. |
| | | | 120, 701-715 |
| sweet | IbMIPS1 | Increased | Hong, et al. (2016). “A |
| potato | | salt and | myo-inositol-1-phosphate synthase gene, |
| | | drought | IbMIPSI, enhances salt and drought tolerance |
| | | tolerance | and stem nematode resistance in transgenic |
| | | | sweet potato”. Plant Biotechnology Journal. |

TABLE L
List of herbicide resistance genes

| | | Gene | |
|---|---|---|---|
| Crop | Gene name | number | Reference |
| wheat | Imi1 | AY210407.1 | Perez-Jones, A., et al. (2006). “Introgression of an |
| | | | imidazolinone-resistance gene from winter wheat |
| | | | (Triticum aestivum L.) into jointed goatgrass (Aegilops |
| | | | cylindrica Host).” Theor Appl Genet114(l): 177-186. |
| wheat | GST Cla47 | AY064480.1 | Theodoulou, F.L., et al. (2003). “Co-induction of |
| | | | glutathione-S-transferases and multi drug resistance |
| | | | associated protein by xenobiotics in wheat.” Pest Manag |
| | | | Sci59(2): 202-214. |
| wheat | GST 19E50 | AY064481.1 | Theodoulou, F.L., et al. (2003). “Co-induction of |
| | | | glutathione-S-transferases and multi drug resistance |
| | | | associated protein by xenobiotics in wheat.” Pest Manag |
| | | | Sci59(2): 202-214. |
| wheat | GST 28e45 | AF479764.1 | Theodoulou, F.L., et al. (2003). “Co-induction of |
| | | | glutathione-S-transferases and multi drug resistance |
| | | | associated protein by xenobiotics in wheat.” Pest Manag |
| | | | Sci59(2): 202-214. |
| wheat | MRP1 | AY064479.1 | Theodoulou, F.L., et al. (2003). “Co-induction of |
| | | | glutathione-S-transferases and multi drug resistance |
| | | | associated protein by xenobiotics in wheat.” Pest Manag |
| | | | Sci59(2): 202-214. |
| wheat | cytochrome | LOC543123 | Busi, R., et al. (2020). “Cinmethylin controls multiple |
| | P450 | | herbicide-resistant Lolium rigidum and its wheat |
| | | | selectivity is P450-based.” Pest Manag Sci76(8): |
| | | | 2601-2608. |
| corn | ZmSCE1b | | Wang, H., et al. (2021). “The maize SUMO conjugating |
| | | | enzyme ZmSCE1b protects plants from paraquat |
| | | | toxicity.” Ecotoxicol Environ Saf 211: 111909. |
| corn | ZmGSTIV | | Sun, L., et al. (2018). “The expression of detoxification |
| | | | genes in two maize cultivars by interaction of |
| | | | isoxadifen-ethyl and nicosulfuron.” Plant Physiol |
| | | | Biochem 129: 101-108. |
| corn | ZmGST6 | | Sun, L., et al. (2018). “The expression of detoxification |
| | | | genes in two maize cultivars by interaction of |
| | | | isoxadifen-ethyl and nicosulfuron.” Plant Physiol |
| | | | Biochem 129: 101-109. |
| corn | ZmGST31 | | Sun, L., et al. (2018). “The expression of detoxification |
| | | | genes in two maize cultivars by interaction of |
| | | | isoxadifen-ethyl and nicosulfuron.” Plant Physiol |
| | | | Biochem 129: 101-110. |
| corn | ZmMRP1 | | Sun, L., et al. (2018). “The expression of detoxification |
| | | | genes in two maize cultivars by interaction of |
| | | | isoxadifen-ethyl and nicosulfuron.” Plant Physiol |
| | | | Biochem 129: 101-111. |
| corn | GSTI | | Li, D., et al. (2017). “Characterization of glutathione |
| | | | S-transferases in the detoxification of metolachlor in |
| | | | two maize cultivars of differing herbicide tolerance.” |
| | | | Pestic Biochem Physiol 143: 265-271. |
| corn | GSTIII | | Li, D., et al. (2017). “Characterization of glutathione |
| | | | S-transferases in the detoxification of metolachlor in |
| | | | two maize cultivars of differing herbicide tolerance.” |
| | | | Pestic Biochem Physiol 143: 265-271. |
| corn | GSTIV | | Li, D., et al. (2017). “Characterization of glutathione |
| | | | S-transferases in the detoxification of metolachlor in |
| | | | two maize cultivars of differing herbicide tolerance.” |
| | | | Pestic Biochem Physiol 143: 265-271. |
| corn | GST5 | | Li, D., et al. (2017). “Characterization of glutathione |
| | | | S-transferases in the detoxification of metolachlor in |
| | | | two maize cultivars of differing herbicide tolerance.” |
| | | | Pestic Biochem Physiol 143: 265-271. |
| corn | GST6 | | Li, D., et al. (2017). “Characterization of glutathione |
| | | | S-transferases in the detoxification of metolachlor in |
| | | | two maize cultivars of differing herbicide tolerance.” |
| | | | Pestic Biochem Physiol 143: 265-271. |
| corn | GST7 | | Li, D., et al. (2017). “Characterization of glutathione |
| | | | S-transferases in the detoxification of metolachlor in |
| | | | two maize cultivars of differing herbicide tolerance.” |
| | | | Pestic Biochem Physiol 143: 265-271. |
| corn | bifunctional | | Mahmoud, M., et al. (2020). “Identification of |
| | 3-dehydroquinate | | Structural Variants in Two Novel Genomes of Maize |
| | dehydratase | | Inbred Lines Possibly Related to Glyphosate |
| | | | Tolerance.” Plants (Basel) 9(4). |
| corn | shikimate | | Mahmoud, M., et al. (2020). “Identification of |
| | dehydrogenase | | Structural Variants in Two Novel Genomes of Maize |
| | | | Inbred Lines Possibly Related to Glyphosate |
| | | | Tolerance.” Plants (Basel) 9(5). |
| corn | chorismate | | Mahmoud, M., et al. (2020). “Identification of |
| | synthase | | Structural Variants in Two Novel Genomes of Maize |
| | | | Inbred Lines Possibly Related to Glyphosate |
| | | | Tolerance.” Plants (Basel) 9(6). |
| corn | (T102I + P10 | | Yu, Q., et al. (2015). “Evolution of a double amino acid |
| | 6S [TIPS]) | | substitution in the 5-enolpyruvylshikimate-3-phosphate |
| | (EPSPS) | | synthase in Eleusine indica conferring high-level |
| | | | glyphosate resistance.” Plant Physiol 167(4): |
| | | | 1440-1447. |
| corn | CYP81A9 | Zm00001 | Liu, X., et al. (2019). “Rapid identification of a |
| | | d013230 | candidate nicosulfuron sensitivity gene (Nss) in maize |
| | | | (Zea mays L.) via combining bulked segregant analysis |
| | | | and RNA-seq.” Theor Appl Genet 132(5): 1351-1361. |
| soybean | GmHRA | | Mathesius, C.A., et al. (2009). “Safety assessment of a |
| | | | modified acetolactate synthase protein (GM-HRA) used |
| | | | as a selectable marker in genetically modified |
| | | | soybeans.” Regul Toxicol Pharmacol 55(3): 309-320. |

“The expression level of a new gene is upregulated” in the present invention means that the expression level of a new gene relative to the endogenous wild-type gene of the corresponding organism is increased, preferably the expression level is increased by at least 0.5 times, at least 1 time, at least 2 times, at least 3 times, at least 4 times or at least 5 times.

The term “gene editing” refers to strategies and techniques for targeted specific modification of any genetic information or genome of living organisms. Therefore, the term includes editing of gene coding regions, but also includes editing of regions other than gene coding regions of the genome. It also includes editing or modifying other genetic information of nuclei (if present) and cells.

The term “CRISPR/Cas nuclease” may be a CRISPR-based nuclease or a nucleic acid sequence encoding the same, including but not limited to: 1) Cas9, including SpCas9, ScCas9, SaCas9, xCas9, VRER-Cas9, EQR-Cas9, SpG-Cas9, SpRY-Cas9, SpCas9-NG, NG-Cas9, NGA-Cas9 (VQR), etc.; 2) Cas12, including LbCpf1, FnCpf1, AsCpf1, MAD7, etc., or any variant or derivative of the aforementioned CRISPR-based nuclease; preferably, wherein the at least one CRISPR-based nuclease comprises a mutation compared to the corresponding wild-type sequence, so that the obtained CRISPR-based nuclease recognizes a different PAM sequence. As used herein, “CRISPR-based nuclease” is any nuclease that has been identified in a naturally occurring CRISPR system, which is subsequently isolated from its natural background, and has preferably been modified or combined into a recombinant construct of interest, suitable as a tool for targeted genome engineering. As long as the original wild-type CRISPR-based nuclease provides DNA recognition, i.e., binding properties, any CRISPR-based nuclease can be used and optionally reprogrammed or otherwise mutated so as to be suitable for various embodiments of the invention.

The term “CRISPR” refers to a sequence-specific genetic manipulation technique that relies on clustered regularly interspaced short palindromic repeats, which is different from RNA interference that regulates gene expression at the transcriptional level.

“Cas9 nuclease” and “Cas9” are used interchangeably herein, and refer to RNA-guided nuclease comprising Cas9 protein or fragment thereof (for example, a protein containing the active DNA cleavage domain of Cas9 and/or the gRNA binding domain of Cas9). Cas9 is a component of the CRISPR/Cas (clustered regularly interspaced short palindrome repeats and associated systems) genome editing system. It can target and cut DNA target sequences under the guidance of guide RNA to form DNA double-strand breaks (DSB).

“Cas protein” or “Cas polypeptide” refers to a polypeptide encoded by Cas (CRISPR-associated) gene. Cas protein includes Cas endonuclease. Cas protein can be a bacterial or archaeal protein. For example, the types I to III CRISPR Cas proteins herein generally originate from prokaryotes; the type I and type III Cas proteins can be derived from bacteria or archaea species, and the type II Cas protein (i.e., Cas9) can be derived from bacterial species. “Cas proteins” include Cas9 protein, Cpfl protein, C2c1 protein, C2c2 protein, C2c3 protein, Cas3, Cas3-HD, Cas5, Cas7, Cas8, Cas10, Cas12a, Cas12b, or a combination or complex thereof.

“Cas9 variant” or “Cas9 endonuclease variant” refers to a variant of the parent Cas9 endonuclease, wherein when associated with crRNA and tracRNA or with sgRNA, the Cas9 endonuclease variant retains the abilities of recognizing, binding to all or part of a DNA target sequence and optionally unwinding all or part of a DNA target sequence, nicking all or part of a DNA target sequence, or cutting all or part of a DNA target sequence. The Cas9 endonuclease variants include the Cas9 endonuclease variants described herein, wherein the Cas9 endonuclease variants are different from the parent Cas9 endonuclease in the following manner: the Cas9 endonuclease variants (when complexed with gRNA to form a polynucleotide-directed endonuclease complex capable of modifying a target site) have at least one improved property, such as, but not limited to, increased transformation efficiency, increased DNA editing efficiency, decreased off-target cutting, or any combination thereof, as compared to the parent Cas9 endonuclease (complexed with the same gRNA to form a polynucleotide-guided endonuclease complex capable of modifying the same target site).

The Cas9 endonuclease variants described herein include variants that can bind to and nick double-stranded DNA target sites when associated with crRNA and tracrRNA or with sgRNA, while the parent Cas endonuclease can bind to the target site and result in double strand break (cleavage) when associated with crRNA and tracrRNA or with sgRNA.

“Guide RNA” and “gRNA” are used interchangeably herein, and refer to a guide RNA sequence used to target a specific gene for correction using CRISPR technology, which usually consists of crRNA and tracrRNA molecules that are partially complementary to form a complex, wherein crRNA contains a sequence that has sufficient complementarity with the target sequence so to hybridize with the target sequence and direct the CRISPR complex (Cas9+crRNA+tracrRNA) to specifically bind to the target sequence. However, it is known in the art that a single guide RNA (sgRNA) can be designed, which contains both the properties of crRNA and tracrRNA.

The terms “single guide RNA” and “sgRNA” are used interchangeably herein, and refer to the synthetic fusion of two RNA molecules, which comprises a fusion of a crRNA (CRISPR RNA) of a variable targeting domain (linked to a tracr pairing sequence hybridized to tracrRNA) and a tracrRNA (trans-activating CRISPR RNA). The sgRNA may comprise crRNA or crRNA fragments and tracrRNA or tracrRNA fragments of the type II CRISPR/Cas system that can form a complex with the type II Cas endonuclease, wherein the guide RNA/Cas endonuclease complex can guide the Cas endonuclease to a DNA target site so that the Cas endonuclease can recognize, optionally bind to the DNA target site, and optionally nick the DNA target site or cut (introduce a single-strand or double-strand break) the DNA target site.

In certain embodiments, the guide RNA(s) and Cas9 can be delivered to a cell as a ribonucleoprotein (RNP) complex. RNP is composed of purified Cas9 protein complexed with gRNA, and it is well known in the art that RNP can be effectively delivered to many types of cells, including but not limited to stem cells and immune cells (Addgene, Cambridge, Mass., Mirus Bio LLC, Madison, Wis.).

The protospacer adjacent motif (PAM) herein refers to a short nucleotide sequence adjacent to a (targeted) target sequence (prespacer) recognized by the gRNA/Cas endonuclease system. If the target DNA sequence is not adjacent to an appropriate PAM sequence, the Cas endonuclease may not be able to successfully recognize the target DNA sequence. The sequence and length of PAM herein can be different depending on the Cas protein or Cas protein complex in use. The PAM sequence can be of any length, but is typically in length of 1, 2, 3, 4, 5, 6, 7, 8, 9, 10, 11, 12, 13, 14, 15, 16, 17, 18, 19 or 20 nucleotides.

Cytochrome P450 enzyme system (CYP) is discovered as a protein that can be bound to CO. In 1958, Klingenberg discovered this pigment protein in rat liver microsomes. Cytochrome P450 was so named because of its maximum absorption value at 450 nm wavelength when combined with CO in its reduced state. As the largest superfamily of oxidoreductases, P450 is widely distributed in the vast majority of organisms, including but not limited to animals, plants, fungi, bacteria, archaea and viruses. Cytochrome P450 enzymes include those reviewed in the following literature: Van Bogaert et al, 2011, FEBS J. 278(2): 206-221, or Urlacherand Girhard, 2011, Trends in Biotechnology 30(1): 26-36, or the following websites:http://drnelson.uthsc.edu/CytochromeP450.html and http://p450.riceblast.snu.ac.kr/index.php?a=view. Its naming is based on the English abbreviation CYP (Cytochrome P450) with numbers+letters+numbers, respectively representing the family, subfamily and individual enzymes.

For some embodiments, the said cytochrome P450s include but not limited to the following as per list:

Example enzymes.

| Family | Gene |
|---|---|
| CYP1 | CYP1A1, CYP1A2, CYP1B1 |
| CYP2 | CYP2A6, CYP2A7, CYP2A13, CYP2B6, |
| | CYP2C8, CYP2C9, CYP2C18, CYP2C19, |
| | CYP2D6, CYP2E1, CYP2F1, CYP2I2, |
| | CYP2R1, CYP2S1, CYP2U1, CYP2W1 |
| CYP3 | CYP3A4, CYP3A5, CYP3A7, CYP3A43 |
| CYP4 | CYP4A11, CYP4A22, CYP4B1, CYP4F2, |
| | CYP4F3, CYP4FB, CYP4F11, CYP4F12, |
| | CYP4F22, CYP4Y2, CYP4X1, CYP4Z1 |
| CYP5 | CYP5A1 |
| CYP7 | CYP7A1, CYP7B1 |
| CYP8 | CYP8A1 (prostacyclin synthase), CYP8B1 |
| | (bile acid biosyntheses) |
| CYP11 | CYP11A1, CYP11B1, CYP11B2 |
| CYP17 | CYP17A1 |
| CYP19 | CYP19A1 |
| CYP20 | CYP20A1 |
| CYP21 | CYP21A2 |
| CYP24 | CYP24A1 |
| CYP26 | CYP26A1, CYP26B1, CYP26C1 |
| CYP27 | CYP27A1 (bile acid biosynthesis), CYP27B1 |
| | (vitamin D3 1-alpha hydroxylase, activates |
| | vitamin D3). CYP27C1 (unknown function) |
| CYP39 | CYP29A1 |
| CYP46 | CYP46A1 |
| CYP51 | CYP51A1 (lancsterol 14-alpha demethylase) |

For some embodiments, the rice cytochrome P450s include but not limited to the following as per list:

| MSU/TIGR locus ID | CYP name |
|---|---|
| LOC_Os01g08800 | CYP96D1 |
| LOC_Os01g08810 | CYP96E1 |
| LOC_Os01g10040 | CYP90D2v1 |
| LOC_Os01g10040 | CYP90D2v2 |
| LOC_Os01g11270 | CYP710A5 |
| LOC_Os01g11280 | CYP710A6 |
| LOC_Os01g11300 | CYP710A7 |
| LOC_Os01g11340 | CYP710A8 |
| LOC_Os01g12740 | CYP71T1 |
| LOC_Os01g12750 | CYP71T2 |
| LOC_Os01g12760 | CYP71T3 |
| LOC_Os01g12770 | CYP71T4 |
| LOC_Os01g24780 | CYP709D1 |
| LOC_Os01g24810 | CYP89D1 |
| LOC_Os01g27890 | CYP71K1 |
| LOC_Os01g29150 | CYP734A6 |
| LOC_Os01g36294 | CYP71C19P |
| LOC_Os01g38110 | CYP76M14 |
| LOC_Os01g41800 | CYP72A31P |
| LOC_Os01g41810 | CYP72A32 |
| LOC_Os01g41820 | CYP72A33 |
| LOC_Os01g43700 | CYP72A17v1 |
| LOC_Os01g43700 | CYP72A17v2 |
| LOC_Os01g43710 | CYP72A18 |
| LOC_Os01g43740 | CYP72A20 |
| LOC_Os01g43750 | CYP72A21 |
| LOC_Os01g43760 | CYP72A22 |
| LOC_Os01g43774 | CYP72A23 |
| LOC_Os01g43844 | CYP72A24 |
| LOC_Os01g43851 | CYP72A25 |
| LOC_Os01g50490 | CYP706C2 |
| LOC_Os01g50530 | CYP711A2 |
| LOC_Os01g50580 | CYP711A3 |
| LOC_Os01g50590 | CYP711A4 |
| LOC_Os01g52790 | CYP72A35 |
| LOC_Os01g58950 | CYP94D13 |
| LOC_Os01g58960 | CYP94D12 |
| LOC_Os01g58970 | CYP94D11 |
| LOC_Os01g58990 | CYP94D10 |
| LOC_Os01g59000 | CYP94D9 |
| LOC_Os01g59020 | CYP94D7 |
| LOC_Os01g59050 | CYP94D6 |
| LOC_Os01g60450 | CYP73A35P |
| LOC_Os01g63540 | CYP86A9 |
| LOC_Os01g63930 | CYP94C3v1 |
| LOC_Os01g63930 | CYP94C3v2 |
| LOC_Os01g72260 | CYP94E2 |
| LOC_Os01g72270 | CYP94E1 |
| LOC_Os01g72740 | CYP71AA3 |
| LOC_Os01g72760 | CYP71AA2 |
| LOC_Os02g01890 | CYP89E1 |
| LOC_Os02g02000 | CYP74F1 |
| LOC_Os02g02230 | CYP51H5 |
| LOC_Os02g07680 | CYP97B4vl |
| LOC_Os02g07680 | CYP97B4v2 |
| LOC_Os02g07680 | CYP97B4v3 |
| LOC_Os02g07680 | CYP97B4v4 |
| LOC_Os02g07680 | CYP97B4v5 |
| LOC_Os02g09190 | CYP71X12 |
| LOC_Os02g09200 | CYP71X11 |
| LOC_Os02g09220 | CYP71X10 |
| LOC_Os02g09240 | CYP71X8 |
| LOC_Os02g09250 | CYP71X7 |
| LOC_Os02g09290 | CYP71X4 |
| LOC_Os02g09310 | CYP71X3 |
| LOC_Os02g09320 | CYP71X2 |
| LOC_Os02g09330 | CYP71X1P |
| LOC_Os02g09390 | CYP71K3 |
| LOC_Os02g09400 | CYP71K4 |
| LOC_Os02g09410 | CYP71K5 |
| LOC_Os02g11020 | CYP734A2 |
| LOC_Os02g12540 | CYP71V5 |
| LOC_Os02g12550 | CYP71V4 |
| LOC_Os02g12680 | CYP74E1 |
| LOC_Os02g12690 | CYP74E2 |
| LOC_Os02g12890 | CYP711A5v1 |
| LOC_Os02g12890 | CYP711A5v2 |
| LOC_Os02g17760 | CYP71U3 |
| LOC_Os02g21810 | CYP51H4 |
| LOC_Os02g26770 | CYP73A40 |
| LOC_Os02g26810 | CYP73A39 |
| LOC_Os02g29720 | CYP76N1P |
| LOC_Os02g29960 | CYP92A15 |
| LOC_Os02g30080 | CYP81L5 |
| LOC_Os02g30090 | CYP81L4 |
| LOC_Os02g30100 | CYP81L3 |
| LOC_Os02g30110 | CYP81L2 |
| LOC_Os02g32770 | CYP71Z5 |
| LOC_Os02g36030 | CYP76M5 |
| LOC_Os02g36070 | CYP76M8 |
| LOC_Os02g36110 | CYP76M17 |
| LOC_Os02g36150 | CYP71Z6 |
| LOC_Os02g36190 | CYP71Z7 |
| LOC_Os02g36280 | CYP76M6 |
| LOC_Os02g38290 | CYP86E1v1 |
| LOC_Os02g38290 | CYP86E1v2 |
| LOC_Os02g38930 | CYP71X13P |
| LOC_Os02g38940 | CYP71X14 |
| LOC_Os02g44654 | CYP86A10v1 |
| LOC_Os02g44654 | CYP86A10v2 |
| LOC_Os02g45280 | CYP87A5 |
| LOC_Os02g47470 | CYP707A5vl |
| LOC_Os02g47470 | CYP707A5v2 |
| LOC_Os02g47470 | CYP707A5v3 |
| LOC_Os02g57290 | CYP97A4v1 |
| LOC_Os02g57290 | CYP97A4v2 |
| LOC_Os02g57290 | CYP97A4v3 |
| LOC_Os02g57290 | CYP97A4v4 |
| LOC_Os02g57810 | CYP715B1 |
| LOC_Os03g02180 | CYP84A6 |
| LOC_Os03g04190 | CYP78A17 |
| LOC_Os03g04530 | CYP96B6 |
| LOC_Os03g04630 | CYP96B2 |
| LOC_Os03g04640 | CYP96B9 |
| LOC_Os03g04650 | CYP96B3 |
| LOC_Os03g04660 | CYP96B5 |
| LOC_Os03g04680 | CYP96B4 |
| LOC_Os03g07250 | CYP704B2 |
| LOC_Os03g12260 | CYP94D15 |
| LOC_Os03g12500 | CYP74A5 |
| LOC_Os03g12660 | CYP90B2 |
| LOC_Os03g14400 | CYP76H4 |
| LOC_Os03g14420 | CYP76H5 |
| LOC_Os03g14560 | CYP76Q1 |
| LOC_Os03g21400 | CYP714B2 |
| LOC_Os03g25150 | CYP75A11 |
| LOC_Os03g25480 | CYP709E1 |
| LOC_Os03g25490 | CYP709E2Pv1 |
| LOC_Os03g25490 | CYP709E2Pv2 |
| LOC_Os03g30420 | CYP78A12 |
| LOC_Os03g37080 | CYP71E6P |
| LOC_Os03g37290 | CYP79A7 |
| LOC_Os03g39540 | CYP71AC3P |
| LOC_Os03g39650 | CYP71W1 |
| LOC_Os03g39690 | CYP71W3 |
| LOC_Os03g39760 | CYP71W4 |
| LOC_Os03g40540 | CYP85A1 |
| LOC_Os03g40600 | CYP78A14 |
| LOC_Os03g44740 | CYP92C21 |
| LOC_Os03g45619 | CYP87C2v1 |
| LOC_Os03g45619 | CYP87C2v2 |
| LOC_Os03g55240 | CYP81A6 |
| LOC_Os03g55260 | CYP81A8 |
| LOC_Os03g55800 | CYP74A4 |
| LOC_Os03g61980 | CYP733A1 |
| LOC_Os03g63310 | CYP71E4 |
| LOC_Os04g01140 | CYP93G1v1 |
| LOC_Os04g01140 | CYP93G1v2 |
| LOC_Os04g03870 | CYP723A2 |
| LOC_Os04g03890 | CYP723A3 |
| LOC_Os04g08824 | CYP79A10 |
| LOC_Os04g08828 | CYP79A9 |
| LOC_Os04g09430 | CYP79A9P |
| LOC_Os04g09920 | CYP99A3 |
| LOC_Os04g10160 | CYP99A2 |
| LOC_Os04g18380 | CYP81M1 |
| LOC_Os04g27020 | CYP71Z1 |
| LOC_Os04g33370 | CYP77A18 |
| LOC_Os04g39430 | CYP724B1 |
| LOC_Os04g40460 | CYP71S2 |
| LOC_Os04g40470 | CYP71S1 |
| LOC_Os04g47250 | CYP86A11 |
| LOC_Os04g48170 | CYP87A6 |
| LOC_Os04g48200 | CYP87B4 |
| LOC_Os04g48210 | CYP87A4v1 |
| LOC_Os04g48210 | CYP87A4v2 |
| LOC_Os04g48460 | CYP704A3 |
| LOC_Os05g01120 | CYP722B1 |
| LOC_Os05g08850 | CYP96D2 |
| LOC_Os05g11130 | CYP90D3 |
| LOC_Os05g12040 | CYP51G3 |
| LOC_Os05g25640 | CYP73A38 |
| LOC_Os05g30890 | CYP72A34 |
| LOC_Os05g31740 | CYP94E3 |
| LOC_Os05g33590 | CYP721B2 |
| LOC_Os05g33600 | CYP721B1 |
| LOC_Os05g34325 | CYP51H6 |
| LOC_Os05g34330 | CYP51H7P |
| LOC_Os05g34380 | CYP51H8 |
| LOC_Os05g35010 | CYP71AD1 |
| LOC_Os05g37250 | CYP94C4 |
| LOC_Os05g40384 | CYP714D1 |
| LOC_Os05g41440 | CYP98A4v1 |
| LOC_Os05g41440 | CYP98A4v2 |
| LOC_Os05g43910 | CYP71R1 |
| LOC_Os06g01250 | CYP93G2 |
| LOC_Os06g02019 | CYP88A5 |
| LOC_Os06g03930 | CYP704A4 |
| LOC_Os06g09210 | CYP709C10 |
| LOC_Os06g09220 | CYP709C11 |
| LOC_Os06g15680 | CYP71R2P |
| LOC_Os06g19070 | CYP76Q2 |
| LOC_Os06g22020 | CYP71C20 |
| LOC_Os06g22340 | CYP89C1 |
| LOC_Os06g24180 | CYP84A7 |
| LOC_Os06g30179 | CYP71AB3 |
| LOC_Os06g30500 | CYP71AB2 |
| LOC_Os06g30640 | CYP76M9 |
| LOC_Os06g36920 | CYP711A6 |
| LOC_Os06g37224 | CYP701A9 |
| LOC_Os06g37300 | CYP701A8 |
| LOC_Os06g37330 | CYP701A19 |
| LOC_Os06g37364 | CYP701A6v1 |
| LOC_Os06g37364 | CYP701A6v2 |
| LOC_Os06g37364 | CYP701A6v3 |
| LOC_Os06g39780 | CYP76M7 |
| LOC_Os06g39880 | CYP734A4 |
| LOC_Os06g41070 | CYP93F1 |
| LOC_Os06g42610 | CYP89B12P |
| LOC_Os06g43304 | CYP71Y7 |
| LOC_Os06g43320 | CYP71Y6 |
| LOC_Os06g43350 | CYP71Y5 |
| LOC_Os06g43370 | CYP71Y4 |
| LOC_Os06g43384 | CYP71Y3 |
| LOC_Os06g43410 | CYP71Y1P |
| LOC_Os06g43420 | CYP71K10 |
| LOC_Os06g43430 | CYP71K9 |
| LOC_Os06g43440 | CYP71K8 |
| LOC_Os06g43480 | CYP71K7P |
| LOC_Os06g43490 | CYP71K6 |
| LOC_Os06g43520 | CYP71AF1 |
| LOC_Os06g45960 | CYP71AC2 |
| LOC_Os06g46680 | CYP77B2 |
| LOC_Os07g11739 | CYP71Z2 |
| LOC_Os07g11870 | CYP71Z21 |
| LOC_Os07g11970 | CYP71Z22 |
| LOC_Os07g19130 | CYP71Q2 |
| LOC_Os07g19210 | CYP71Q1 |
| LOC_Os07g23570 | CYP709C9 |
| LOC_Os07g23710 | CYP709C12P |
| LOC_Os07g26870 | CYP89G1 |
| LOC_Os07g28160 | CYP51H1 |
| LOC_Os07g29960 | CYP87B5 |
| LOC_Os07g33440 | CYP728B3 |
| LOC_Os07g33480 | CYP728C9v1 |
| LOC_Os07g33480 | CYP728C9v2 |
| LOC_Os07g33540 | CYP728C7 |
| LOC_Os07g33550 | CYP728C5 |
| LOC_Os07g33560 | CYP728C4 |
| LOC_Os07g33580 | CYP728C3 |
| LOC_Os07g33610 | CYP728C1v1 |
| LOC_Os07g33610 | CYP728Clv2 |
| LOC_Os07g33620 | CYP728B1 |
| LOC_Os07g37970 | CYP51H9 |
| LOC_Os07g37980 | CYP51G4P |
| LOC_Os07g41240 | CYP78A13 |
| LOC_Os07g44110 | CYP709C8 |
| LOC_Os07g44130 | CYP709C6 |
| LOC_Os07g44140 | CYP709C5 |
| LOC_Os07g45000 | CYP727A1 |
| LOC_Os07g45290 | CYP734A5 |
| LOC_Os07g48330 | CYP714B1 |
| LOC_Os08g01450 | CYP71C12 |
| LOC_Os08g01470 | CYP71C13P |
| LOC_Os08g01490 | CYP71C17 |
| LOC_Os08g01510 | CYP71C15 |
| LOC_Os08g01520 | CYP71C16 |
| LOC_Os08g03682 | CYP703A3 |
| LOC_Os08g05610 | CYP89C8P |
| LOC_Os08g05620 | CYP89C9 |
| LOC_Os08g12990 | CYP76H11 |
| LOC_Os08g16260 | CYP96B8 |
| LOC_Os08g16430 | CYP96B7 |
| LOC_Os08g33300 | CYP735A3 |
| LOC_Os08g35510 | CYP92A12 |
| LOC_Os08g36310 | CYP76M1 |
| LOC_Os08g36860 | CYP707A6 |
| LOC_Os08g39640 | CYP76M11P |
| LOC_Os08g39660 | CYP76M10 |
| LOC_Os08g39694 | CYP76M4Pv1 |
| LOC_Os08g39694 | CYP76M4Pv2 |
| LOC_Os08g39694 | CYP76M4Pv3 |
| LOC_Os08g39730 | CYP76M2 |
| LOC_Os08g43390 | CYP78A15 |
| LOC_Os08g43440 | CYP706C1 |
| LOC_Os09g08920 | CYP92A13 |
| LOC_Os09g08990 | CYP92A14 |
| LOC_Os09g10340 | CYP71V2 |
| LOC_Os09g21260 | CYP728A1 |
| LOC_Os09g23820 | CYP735A4 |
| LOC_Os09g26940 | CYP92A11 |
| LOC_Os09g26960 | CYP92A9 |
| LOC_Os09g26970 | CYP92A8 |
| LOC_Os09g26980 | CYP92A7 |
| LOC_Os09g27500 | CYP76L1 |
| LOC_Os09g27510 | CYP76K1 |
| LOC_Os09g28390 | CYP707A37 |
| LOC_Os09g35940 | CYP78A16 |
| LOC_Os09g36070 | CYP71T8 |
| LOC_Os09g36080 | CYP71AK2 |
| LOC_Os10g05020 | CYP89B11 |
| LOC_Os10g05490 | CYP76P1 |
| LOC_Os10g08319 | CYP76H9 |
| LOC_Os10g08474 | CYP76H8 |
| LOC_Os10g08540 | CYP76H6 |
| LOC_Os10g09090 | CYP76V1 |
| LOC_Os10g09160 | CYP71AB1 |
| LOC_Os10g16974 | CYP75B11 |
| LOC_Os10g17260 | CYP75B3 |
| LOC_Os10g21050 | CYP76P3 |
| LOC_Os10g23130 | CYP729A2 |
| LOC_Os10g23180 | CYP729A1v1 |
| LOC_Os10g23180 | CYP729A1v2 |
| LOC_Os10g26340 | CYP78A11 |
| LOC_Os10g30380 | CYP71Z3 |
| LOC_Os10g30390 | CYP71Z4 |
| LOC_Os10g30410 | CYP71Z8 |
| LOC_Os10g34480 | CYP86B3 |
| LOC_Os10g36740 | CYP89F1 |
| LOC_Os10g36848 | CYP84A5 |
| LOC_Os10g36960 | CYP89B10 |
| LOC_Os10g36980 | CYP89B9 |
| LOC_Os10g37020 | CYP89B8P |
| LOC_Os10g37034 | CYP89B7P |
| LOC_Os10g37050 | CYP89B6 |
| LOC_Os10g37070 | CYP89B5P |
| LOC_Os10g37100 | CYP89B4 |
| LOC_Os10g37110 | CYP89B3 |
| LOC_Os10g37120 | CYP89B2 |
| LOC_Os10g37160 | CYP89B1 |
| LOC_Os10g38090 | CYP704A7 |
| LOC_Os10g38110 | CYP704A5v1 |
| LOC_Os10g38110 | CYP704A5v2 |
| LOC_Os10g38120 | CYP704A6 |
| LOC_Os10g39930 | CYP97C2v1 |
| LOC_Os10g39930 | CYP97C2v2 |
| LOC_Os11g02710 | CYP714C16P |
| LOC_Os11g04290 | CYP94D5 |
| LOC_Os11g04310 | CYP94D4 |
| LOC_Os11g04710 | CYP90A3 |
| LOC_Os11g05380 | CYP94C2 |
| LOC_Os11g18570 | CYP87B1 |
| LOC_Os11g27730 | CYP71C32 |
| LOC_Os11g28060 | CYP71C33 |
| LOC_Os11g29290 | CYP94B4 |
| LOC_Os11g29720 | CYP78D1 |
| LOC_Os11g32240 | CYP51G1 |
| LOC_Os11g41680 | CYP71K11 |
| LOC_Os11g41710 | CYP71K12 |
| LOC_Os12g02630 | CYP714C1 |
| LOC_Os12g02640 | CYP714C2 |
| LOC_Os12g04100 | CYP94D63 |
| LOC_Os12g04110 | CYP94D64 |
| LOC_Os12g04480 | CYP90A19 |
| LOC_Os12g05440 | CYP94C79 |
| LOC_Os12g09500 | CYP76P2 |
| LOC_Os12g09790 | CYP76M13 |
| LOC_Os12g16720 | CYP71P1 |
| LOC_Os12g18820 | CYP87C5P |
| LOC_Os12g25660 | CYP94B5 |
| LOC_Os12g32850 | CYP71E5 |
| LOC_Os12g39240 | CYP81N1 |
| LOC_Os12g39300 | CYP81N1P |
| LOC_Os12g39310 | CYP81P1 |
| LOC_Os12g44290 | CYP71V3 |

As used herein, ATP-binding cassette (ABC) transporter family are a family of membrane transporter proteins that regulate the transport of a wide variety of pharmacological agents, potentially toxic drugs, and xenobiotics, as well as anions. ABC transporters are homologous membrane proteins that bind and use cellular adenosine triphosphate (ATP) for their specific activities. And the ATP-binding cassette (ABC) transporter proteins comprise a large family of prokaryotic and eukaryotic membrane proteins involved in the energy-dependent transport of a wide range of substrates across membranes (Higgins, C. F. et al., Ann. Rev. Cell Biol., 8:67-113 (1992)). In eukaryotes, ABC transport proteins typically consist of four domains that include two conserved ATP-binding domains and two transmembrane domains (Hyde et al., Nature, 346:362-5 (1990)).

As used herein, NAC transcription factors are unique transcription factors in plants and are numerous and widely distributed in terrestrial plants. They constitute one of the largest transcription factor families and play an important role in multiple growth development and stress response processes. Wherein, NAC is an acronym derived from the names of the three genes first described as containing a NAC domain, namely NAM (no apical meristem), ATAF1,2 and CUC2 (cup-shaped cotyledon).

As used herein, wherein, the Myb gene was found to encode a transcription factor (Biedenkapp H., et al., 1988, Nature, 335: 835-837), represent a family comprising many related genes, and exist in a wide variety of species, including yeast, nematodes, insects and plants, as well as vertebrates (Masaki Iwabuchi and Kazuo Shinozaki, Shokubutsu genomu kinou no dainamizumu: tensha inshi ni yoru hatsugen seigyo (Dynamism of Plant Genome functions: Expression control by transcriptional factor), Springer Japan, 2001). Plant transcription factor MYB (v-myb avian myeloblastosis viral oncogene homolog) is a type of transcription factor discovered in recent years that is related to the regulation of plant growth and development, physiological metabolism, cell morphology and pattern formation and other physiological processes. It is ubiquitous in plants and is also one of the largest transcription families in plants, MYB transcription factors play an important role in plant metabolism and regulation. Most MYB proteins contain a Myb domain composed of amino acid residues at the N-terminus. According to the structural characteristics of this highly conserved domain, MYB transcription factors can be divided into four categories: 1R-MYB/MYB-related; R2R3-MYB; 3R-MYB; 4R-MYB (4 repetitions of R1/R2). MYB transcription factors have a variety of biological functions and are widely involved in the growth and development of plant roots, stems, leaves, and flowers. At the same time, the MYB gene family also responds to abiotic stress processes such as drought, salinity, and cold damage. In addition, MYB transcription factors are also closely related to the quality of certain cash crops.

As used herein, the family of MADS transcription factors that play critical roles in diverse developmental process in plants including flower and seed development (Minster, et al., 2002; Parenicova, et al., 2003). MADS protein is composed of domains such as MADS (M), Intervening (I), Keratin 2 like (K) and C2 terminal (C), which belong to domain proteins.

As used herein, the DREB (dehydration responsive element binding protein) type transcription factor is a subfamily of the AP2/EREBP (APETALA2/an ethylene-responsive element binding protein) transcription factor family. It has a conserved AP2 domain and can specifically combine with DRE cis-acting elements in the promoter region of stress resistance genes to regulate the expression of a series of downstream stress response genes under conditions of low temperature, drought, saline-alkali and so on. It is a key regulatory factor in stress adaptation.

As used herein, the bZIP (basic region/leucine zipper) family of transcription factors comprises the simplest motif that nature uses for targeting specific DNA sites: a pair of short α-helices that recognize the DNA major groove with sequence-specificity and high affinity (Struhl, K., Ann. Rev. Biochem., 1989, 58, 1051; Landschulz, W. H., et al., Science, 1988, 240, 1759-1764).

As used herein, plant bZIP transcription factors are a class of proteins that are widely distributed in eukaryotes and relatively conserved. Its basic region is highly conserved and contains about 20 amino acid residues. According to the difference in the structure of bZIP, it can be divided into 10 subfamilies The transcription factors of different subgroups perform different functions, mainly including the expression of plant seed storage genes, the regulation of plant growth and development, light signal transduction, disease prevention, stress response and ABA sensitivity and other signal responses.

As used herein, Glutathione-S-Transferases (GSTs) family are a large family of enzymes ubiquitously expressed in animals, plants and microorganism. It is a superfamily of enzymes that are encoded by multiple genes and have multiple functions. They are combined with harmful heterologous substances or oxidation products through glutathione to promote the metabolism, regional isolation or elimination of such substances, and involved in cellular defense against a broad spectrum of cytotoxic agents (see Gate and Tew, Expert Opin. Ther. Targets 5: 477, 2001). Over 400 different GST sequences have been identified and based on their genetic characteristics and substrate specificity can be classified in four different classes α, μ, π, and θ (see Mannervik et al., Biochem. J. 282:305, 1992). Each allelic variant encoded at the same gene locus is distinguished by a letter. According to the homology and gene structure characteristics of plant proteins, the GST family is divided into 8 subfamilies: F (Phi), U (Tau), T (Theta), Z (Zeta), L (Lambda), DHAR, EF1Bγ and TCHQD. The F and U families are unique to plants. Compared with other subfamilies, they have the most members and the most abundant content. Soluble GST is mainly distributed in the cytoplasm, a few in chloroplasts and microbodies, and a small amount in the nucleus and apoplasts. Plant GST was first discovered in corn (Zea mays L.), and subsequently found in plants such as Arabidopsis thaliana, soybean (Glycine max), rice (Oryza sativa L.), and tobacco (Nicotiana tabacum L.).

As used herein, the term “organism” includes animals, plants, fungi, bacteria, and the like.

As used herein, the term “host cell” includes plant cells, animal cells, fungal cells, bacterial cells, and the like.

In the present invention, the term “animal” includes any member of the animal kingdom, for example, invertebrates and vertebrates. Invertebrates include but not limited to protozoa (such as amoeba), helminthes, molluscs (such as escargots, snails, freshwater mussels, oysters and devilfishes), arthropods (such as insects, spiders, and crabs), etc.; vertebrates include but not limited to fishes (such as zebrafish, salmon, crucian carp, carp or tilapia and other edible economic fish that can be raised artificially), amphibians (such as frogs, toads and newts), reptiles (such as snakes, lizards, iguanas, turtles and crocodiles), birds (such as chickens, geese, ducks, turkeys, ostriches, quails, pheasants, parrots, finches, hawks, eagles, kites, vultures, harriers, ospreys, owls, crows, guinea fowls, pigeons, emus and cassowaries), mammals (such as humans, non-human primates (such as lemurs, tarsier, monkeys, apes and orangutans), pigs, cattle, sheep, horses, camels, rabbits, kangaroos, deer, polar bears, canines (such as dogs, wolves, foxes and jackals), felines (such as lions, tigers, cheetahs, lynxes and cats) and rodents (such as mice, rats, hamsters and guinea pigs)] etc. The term “non-human” does not include humans.

The term “animal” also includes individual animals at every developmental stage (including newborn, embryo, and fetus stage).

The term “fungus” refers to any member of eukaryotic organisms generated by saprophytic and parasitic spores. Generally, they are filamentous organisms and previously they are classified as chlorophyll deficiency plants, including but not limited to basidiomycotina, deuteromycotina, ascomycotina, mastigomycotina, zygomycotina, etc. However, it should be understood that the fungal classification is constantly evolving, and as a result, the specific definition of the fungal kingdom might be adjusted in the future. The macro-fungi can be divided into four categories: edible fungi, medicinal fungi, poisonous fungi and fungi with unknown uses. Most of the edible fungi and medicinal fungi belong to basidiomycotina, for example, Tremella fuciformis, Phlogiotis helvelloides, Tremella aurantialba, Auricularia auricular, Auricularia polytricha, Auricularia delicate, Auricularia messenterica, Auricularia rugosissima, Calocera cornea, Fistulina hepatica, Poria cocos, Grifola frondosa, Grifola umbellate, Ganoderma applanatum, Coriolus versicolor, Ganoderma capense, Ganoderma lucifum, Ganoderma cochlear, Ganoderma lobatum, Ganoderma tsugae, Ganderma sinense, Polyporus rhinoceros, Omphalia lapidescens, Phellinus baumii, Cryptoporus volvatus, Pycnoporus cinnabarinus, Fuscoporus obliqus, Sparassis crispa, Hericium erinaceus, Thelephora vialis, Ramaria flava, Ramaria botrytoides, Ramaria stricta, Ramaria botrytis, Clavicorona pyxidata, Clavulina cinerea, Cantharellus cibarius, Hydnum repandum, Lycoperdon perlatum, Lycoperdon Polymorphum, Lycoperdon pusllum, Lycoperdon aurantium, Lycoperdon flavidum, Lycoperdon poleroderma, Lycoperdon verrucosum, Boletus albidus, Boletus aereus, Boletus rubellus, Suillus grevillea, Suillus granulatus, Suillus luteus, Fistulina hepatica, Russula integra, Russula alutacea, Russula zoeteus, Russula Viresceu, Pleurotus citrinopileatus, Pleurotus ostreatus, Pleurotus sapidus, Pleurotus ferulae, Pleurotus abalonus, Pleurotus cornucopiae, Pleurotus cystidiosus, Pleurotus djamor, Pleurotus salmoneostramineus, Pleurotus eryngii (DC. ex Fr.) Quel. var. eryngii, Pleurotus eryngii (DC. ex Fr.) Quel.var. ferulae Lanzi, Pleurotus nebrodensis, Pleurotus ostreatus, Pleurotus florida, Pleurotus pulmonarius, Pleurotus tuber-regium, Hohenbuchelia serotine, Agaricus bisporus, Agaricus arvensis, Agaricus blazei, Tricholoma matsutake, Tricholoma gambosum, Tricholoma conglobatum, Tricholoma album, Tricholoma mongolicum, Armillaria mellea, Armillariella ventricosa, Armillariella mucida, Armillariella tabescens, Collybia radicata, Collybia radicata (Relh.ex Fr.) Quel. var. furfuracea PK., Marasmius androsaceus, Termitomyces albuminosus, Tricholoma giganteum, Hypsizigus marmoreus, Lepista sordida, Lyophyllum ulmarium, Lyophyllum shimeji, Flammulina velutipes, Cortinarius armillatus, Amanita caesarea, Amanita caesarea (Scop. ex Fr.) Pers. ex Schw. var. alba Gill, Amanita strobiliformis, Amanita vaginata, Volvariella volvacea, Pholiota adiposa, Pholiot squarrosa, Pholiot mutabilis, Pholiota nameko, Stropharia rugoso-annulata, Coprinus sterquilinus, Coprinus fuscesceus, Coprinus atramentarius, Coprinus comatus, Coprinus ovatus, Dictyophora indusiata, Dictyophora duplicate, Dictyophora echino-volvata, Schizphylhls commne, Agrocybe cylindracea, Lentinus edodes; some are Ascomycotina, for example, Morchella esculenta, Cordyceps sinensis, Cordyceps militaris, Claviceps purpurea, Cordyceps sobolifera, Engleromyces geotzii, Podostroma yunnansis, Shiraia bambusiicola, Hypocrella bambusea, Xylaria nigripes, Tuber spp.

In the present invention, the “plant” should be understood to mean any differentiated multicellular organism capable of performing photosynthesis, in particular monocotyledonous or dicotyledonous plants, for example, (1) food crops: Oryza spp., like Oryza sativa, Oryza latifolia, Oryza sativa, Oryza glaberrima; Triticum spp., like Triticum aestivum, T. Turgidum ssp. durum; Hordeum spp., like Hordeum vulgare, Hordeum arizonicum; Secale cereale; Avena spp., like Avena sativa, Avena fatua, Avena byzantine, Avena fatua var.sativa, Avena hybrida; Echinochloa spp., like Pennisetum glaucum, Sorghum, Sorghum bicolor, Sorghum vulgare, Triticale, Zea mays or Maize, Millet, Rice, Foxtail millet, Proso millet, Sorghum bicolor, Panicum, Fagopyrum spp., Panicum miliaceum, Setaria italica, Zizania palustris, Eragrostis tef, Panicum miliaceum, Eleusine coracana; (2) legume crops: Glycine spp. like Glycine max, Soja hispida, Soja max, Vicia spp., Vigna spp., Pisum spp., field bean, Lupinus spp., Vicia, Tamarindus indica, Lens culinaris, Lathyrus spp., Lablab, broad bean, mung bean, red bean, chickpea; (3) oil crops: Arachis hypogaea, Arachis spp, Sesamum spp., Helianthus spp. like Helianthus annuus, Elaeis like Eiaeis guineensis, Elaeis oleifera, soybean, Brassicanapus, Brassica oleracea, Sesamum orientale, Brassica juncea, Oilseed rape, Camellia oleifera, oil palm, olive, castor-oil plant, Brassica napus L., canola; (4) fiber crops: Agave sisalana, Gossypium spp. like Gossypium, Gossypium barbadense, Gossypium hirsutum, Hibiscus cannabinus, Agave sisalana, Musa textilis Nee, Linum usitatissimum, Corchorus capsularis L, Boehmeria nivea (L.), Cannabis sativa, Cannabis sativa; (5) fruit crops: Ziziphus spp., Cucumis spp., Passiflora edulis, Vitis spp., Vaccinium spp., Pyrus communis, Prunus spp., Psidium spp., Punica granatum, Malus spp., Citrullus lanatus, Citrus spp., Ficus carica, Fortunella spp., Fragaria spp., Crataegus spp., Diospyros spp., Eugenia unifora, Eriobotrya japonica, Dimocarpus longan, Carica papaya, Cocos spp., Averrhoa carambola, Actinidia spp., Prunus amygdalus, Musa spp. (Musa acuminate), Persea spp. (Persea Americana), Psidium guajava, Mammea Americana, Mangifera indica, Canarium album (Oleaeuropaea), Caricapapaya, Cocos nucifera, Malpighia emarginata, Manilkara zapota, Ananas comosus, Annona spp., Citrus reticulate (Citrus spp.), Artocarpus spp., Litchi chinensis, Ribes spp., Rubus spp., pear, peach, apricot, plum, red bayberry, lemon, kumquat, durian, orange, strawberry, blueberry, hami melon, muskmelon, date palm, walnut tree, cherry tree; (6) rhizome crops: Manihot spp., Ipomoea batatas, Colocasia esculenta, tuber mustard, Allium cepa (onion), eleocharis tuberose (water chestnut), Cyperus rotundus, Rhizoma dioscoreae; (7) vegetable crops: Spinacia spp., Phaseolus spp., Lactuca sativa, Momordica spp, Petroselinum crispum, Capsicum spp., Solanum spp. (such as Solanum tuberosum, Solanum integrifolium, Solanum lycopersicum), Lycopersicon spp. (such as Lycopersicon esculentum, Lycopersicon lycopersicum, Lycopersicon pyriforme), Macrotyloma spp., Kale, Luffa acutangula, lentil, okra, onion, potato, artichoke, asparagus, broccoli, Brussels sprouts, cabbage, carrot, cauliflower, celery, collard greens, squash, Benincasa hispida, Asparagus officinalis, Apium graveolens, Amaranthus spp., Allium spp., Abelmoschus spp., Cichorium endivia, Cucurbita spp., Coriandrum sativum, B. carinata, Rapbanus sativus, Brassica spp. (such as Brassica napus, Brassica rapa ssp., canola, oilseed rape, turnip rape, turnip rape, leaf mustard, cabbage, black mustard, canola (rapeseed), Brussels sprout, Solanaceae (eggplant), Capsicum annuum (sweet pepper), cucumber, luffa, Chinese cabbage, rape, cabbage, calabash, Chinese chives, lotus, lotus root, lettuce; (8) flower crops: Tropaeolum minus, Tropaeolum majus, Canna indica, Opuntia spp., Tagetes spp., Cymbidium (orchid), Crinum asiaticum L., Clivia, Hippeastrum rutilum, Rosa rugosa, Rosa Chinensis, Jasminum sambac, Tulipa gesneriana L., Cerasus sp., Pharbitis nil (L.) Choisy, Calendula officinalis L., Nelumbo sp., Bellis perennis L., Dianthus caryophyllus, Petunia hybrida, Tulipa gesneriana L., Lilium brownie, Prunus mume, Narcissus tazetta L., Jasminum nudiflorum Lindl., Primula malacoides, Daphne odora, Camellia japonica, Michelia alba, Magnolia liliiflora, Viburnum macrocephalum, Clivia miniata, Malus spectabilis, Paeonia suffruticosa, Paeonia lactiflora, Syzygium aromaticum, Rhododendron simsii, Rhododendron hybridum, Michelia figo (Lour.) Spreng., Cercis chinensis, Kerria japonica, Weigela florida, Fructus forsythiae, Jasminum mesnyi, Parochetus communis, Cyclamen persicum Mill., Phalaenophsis hybrid, Dendrobium nobile, Hyacinthus orientalis, Iris tectorum Maxim, Zantedeschia aethiopica, Calendula officinalis, Hippeastrum rutilum, Begonia semperflorenshybr, Fuchsia hybrida, Begonia maculata Raddi, Geranium, Epipremnum aureum; (9) medicinal crops: Carthamus tinctorius, Mentha spp., Rheum rhabarbarum, Crocus sativus, Lycium chinense, Polygonatum odoratum, Polygonatum Kingianum, Anemarrhena asphodeloides Bunge, Radix ophiopogonis, Fritillaria cirrhosa, Curcuma aromatica, Amomum villosum Lour., Polygonum multiflorum, Rheum officinale, Glycyrrhiza uralensis Fisch, Astragalus membranaceus, Panax ginseng, Panax notoginseng, Acanthopanax gracilistylus, Angelica sinensis, Ligusticum wallichii, Bupleurum sinenses DC., Datura stramonium Linn., Datura metel L., Mentha haplocalyx, Leonurus sibiricus L., Agastache rugosus, Scutellaria baicalensis, Prunella vulgaris L., Pyrethrum carneum, Ginkgo biloba L., Cinchona ledgeriana, Hevea brasiliensis (wild), Medicago sativa Linn, Piper Nigrum L., Radix Isatidis, Atractylodes macrocephala Koidz; (10) raw material crops: Hevea brasiliensis, Ricinus communis, Vernicia fordii, Morus alba L., Hops Humulus lupulus, Betula, Alnus cremastogyne Burk., Rhus vemiciflua stokes; (11) pasture crops: Agropyron spp., Trifolium spp., Miscanthus sinensis, Pennisetum sp., Phalaris arundinacea, Panicum virgatum, prairiegrasses, Indiangrass, Big bluestem grass, Phleum pratense, turf, cyperaceae (Kobresia pygmaea, Carex pediformis, Carex humilis), Medicago sativa Linn, Phleum pratense L., Medicago sativa, Melilotus suavcolen, Astragalus sinicus, Crotalaria juncea, Sesbania cannabina, Azolla imbircata, Eichhornia crassipes, Amorpha fruticosa, Lupinus micranthus, Trifolium, Astragalus adsurgens pall, Pistia stratiotes linn, Alternanthera philoxeroides, Lolium; (12) sugar crops: Saccharum spp., Beta vulgaris; (13) beverage crops: Camellia sinensis, Camellia Sinensis, tea, Coffee (Coffea spp.), Theobroma cacao, Humulus lupulus Linn.; (14) lawn plants: Ammophila arenaria, Poa spp. (Poa pratensis (bluegrass)), Agrostis spp. (Agrostis matsumurae, Agrostis palustris), Lolium spp. (Lolium), Festuca spp. (Festuca ovina L.), Zoysia spp. (Zoysia japonica), Cynodon spp. (Cynodon dactylon/bermudagrass), Stenotaphrum secunda tum (Stenotaphrum secundatum), Paspalum spp., Eremochloa ophiuroides (centipedegrass), Axonopus spp. (carpetweed), Bouteloua dactyloides (buffalograss), Bouteloua var. spp. (Bouteloua gracilis), Digitaria sanguinalis, Cyperus rotundus, Kyllinga brevifolia, Cyperusa muricus, Erigeron canadensis, Hydrocotylesibthorpioides, Kummerowia striata, Euphorbia humifusa, Viola arvensis, Carex rigescens, Carex heterostachya, turf; (15) tree crops: Pinus spp., Salix spp., Acer spp., Hibiscus spp., Eucalyptus spp., Ginkgo biloba, Bambusa sp., Populus spp., Prosopis spp., Quercus spp., Phoenix spp., Fagus spp., Ceiba pentandra, Cinnamomum spp., Corchorus spp., Phragmites australis, Physalis spp., Desmodium spp., Populus, Hedera helix, Populus tomentosa Carr, Viburnum odoratissinum, Ginkgo biloba L., Quercus, Ailanthus altissima, Schima superba, Ilex pur-purea, Platanus acerifolia, Ligustrum lucidum, Buxus megistophylla Levl., Dahurian larch, Acacia mearnsii, Pinus massoniana, Pinus khasys, Pinus yunnanensis, Pinus finlaysoniana, Pinus tabuliformis, Pinus koraiensis, Juglans nigra, Citrus limon, Platanus acerifolia, Syzygium jambos, Davidia involucrate, Bombax malabarica L., Ceiba pentandra (L.), Bauhinia blakeana, Albizia saman, Albizzia julibrissin, Erythrina corallodendron, Erythrina indica, Magnolia gradiflora, Cycas revolute, Lagerstroemia indica, coniferous, macrophanerophytes, Frutex; (16) nut crops: Bertholletia excelsea, Castanea spp., Corylus spp., Carya spp., Juglans spp., Pistacia vera, Anacardium occidentale, Macadamia (Macadamia integrifolia), Carya illinoensis Koch, Macadamia, Pistachio, Badam, other plants that produce nuts; (17) others: Arabidopsis thaliana, Brachiaria eruciformis, Cenchrus echinatus, Setaria faberi, Eleusine indica, Cadaba farinose, algae, Carex elata, ornamental plants, Carissa macrocarpa, Cynara spp., Daucus carota, Dioscorea spp., Erianthus sp., Festuca arundinacea, Hemerocallis fulva, Lotus spp., Luzula sylvatica, Medicago sativa, Melilotus spp., Morms nigra, Nicotiana spp., Olea spp., Ornithopus spp., Pastinaca sativa, Sambucus spp., Sinapis sp., Syzygium spp., Tripsacum dactyloides, Triticosecale rimpaui, Viola odorata, and the like.

In a specific embodiment, the plant is selected from rice, maize, wheat, soybean, sunflower, sorghum, rape, alfalfa, cotton, barley, millet, sugarcane, tomato, tobacco, cassava, potato, sweet potato, Chinese cabbage, cabbage, cucumber, Chinese rose, Scindapsus aureus, watermelon, melon, strawberry, blueberry, grape, apple, citrus, peach, pear, banana, etc.

As used herein, the term “plant” includes a whole plant and any progeny, cell, tissue or part of plant. The term “plant part” includes any part of a plant, including, for example, but not limited to: seed (including mature seed, immature embryo without seed coat, and immature seed); plant cutting; plant cell; plant cell culture; plant organ (e.g., pollen, embryo, flower, fruit, bud, leaf, root, stem, and related explant). Plant tissue or plant organ can be seed, callus tissue, or any other plant cell population organized into a structural or functional unit. Some plant cells or tissue cultures can regenerate a plant that has the physiological and morphological characteristics of the plant from which the cell or tissue is derived, and can regenerate a plant that has substantially the same genotype as the plant. In contrast, some plant cells cannot regenerate plants. The regenerable cells in plant cells or tissue cultures can be embryos, protoplasts, meristematic cells, callus, pollen, leaves, anthers, roots, root tips, silks, flowers, kernels, ears, cobs, husks, or stems.

The plant parts comprise harvestable parts and parts that can be used to propagate offspring plants. The plant parts that can be used for propagation include, for example, but not limited to: seeds, fruits, cuttings, seedlings, tubers and rootstocks. The harvestable parts of plants can be any of useful parts of plants, including, for example, but not limited to: flowers, pollen, seedlings, tubers, leaves, stems, fruits, seeds and roots.

The plant cells are the structural and physiological units of plants. As used herein, the plant cells include protoplasts and protoplasts with partial cell walls. The plant cells may be in a form of isolated single cells or cell aggregates (e.g., loose callus and cultured cells), and may be part of higher order tissue units (e.g., plant tissues, plant organs, and intact plants). Therefore, the plant cells can be protoplasts, gamete-producing cells, or cells or collection of cells capable of regenerating a whole plant. Therefore, in the embodiments herein, a seed containing a plurality of plant cells and capable of regenerating into a whole plant is considered as a “plant part”.

As used herein, the term “protoplast” refers to a plant cell whose cell wall is completely or partially removed and whose lipid bilayer membrane is exposed. Typically, the protoplast is an isolated plant cell without cell wall, which has the potential to regenerate a cell culture or a whole plant.

The plant “offspring” includes any subsequent generations of the plant.

The terms “inhibitory herbicide tolerance” and “inhibitory herbicide resistance” can be used interchangeably, and both refer to tolerance and resistance to an inhibitory herbicide. “Improvement in tolerance to inhibitory herbicide” and “improvement in resistance to inhibitory herbicide” mean that the tolerance or resistance to the inhibitory herbicide is improved as compared to a plant containing the wild-type gene.

Generally, if the herbicidal compounds as described herein, which can be employed in the context of the present invention are capable of forming geometrical isomers, for example E/Z isomers, it is possible to use both, the pure isomers and mixtures thereof, in the compositions according to the invention. If the herbicidal compounds as described herein have one or more centers of chirality and, as a consequence, are present as enantiomers or diastereomers, it is possible to use both, the pure enantiomers and diastereomers and their mixtures, in the compositions according to the invention. If the herbicidal compounds as described herein have ionizable functional groups, they can also be employed in the form of their agriculturally acceptable salts. Suitable are, in general, the salts of those cations and the acid addition salts of those acids whose cations and anions, respectively, have no adverse effect on the activity of the active compounds. Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, methylammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy)eth-1-ylammonium (diglycolamine salt), di(2-hydroxyeth-1-yl)ammonium (diolamine salt), tris(2-hydroxyethyl)ammonium (trolamine salt), tris(2-hydroxypropyl)ammonium, benzyltrimethylammonium, benzyltriethylammonium, N,N,N-trimethylethanolammonium (choline salt), furthermore phosphonium ions, sulfonium ions, preferably tri(C₁-C₄-alkyl)sulfonium, such as tri-methylsulfonium, and sulfoxonium ions, preferably tri(C₁-C₄-alkyl)sulfoxonium, and finally the salts of polybasic amines such as N,N-bis-(3-aminopropyl)methylamine and diethylenetri amine. Anions of useful acid addition salts are primarily chloride, bromide, fluoride, iodide, hydrogensulfate, methylsulfate, sulfate, dihydrogenphosphate, hydrogenphosphate, nitrate, bi-carbonate, carbonate, hexafluorosilicate, hexafluorophosphate, benzoate and also the anions of C₁-C₄-alkanoic acids, preferably formate, acetate, propionate and butyrate.

The herbicidal compounds as described herein having a carboxyl group can be employed in the form of the acid, in the form of an agriculturally suitable salt as mentioned above or else in the form of an agriculturally acceptable derivative, for example as amides, such as mono- and di-C₁-C₆-alkylamides or arylamides, as esters, for example as allyl esters, propargyl esters, C₁-C₁₀-alkyl esters, alkoxyalkyl esters, tefuryl ((tetra-hydrofuran-2-yl)methyl) esters and also as thioesters, for example as C₁-C₁₀-alkylthio esters. Preferred mono- and di-C₁-C₆-alkylamides are the methyl and the dimethylamides. Preferred arylamides are, for example, the anilides and the 2-chloroanilides. Preferred alkyl esters are, for example, the methyl, ethyl, propyl, isopropyl, butyl, isobutyl, pentyl, mexyl (1-methyl hexyl), meptyl (1-methylheptyl), heptyl, octyl or isooctyl (2-ethylhexyl) esters. Preferred C₁-C₄-alkoxy-C₁-C₄-alkyl esters are the straight-chain or branched C₁-C₄-alkoxy ethyl esters, for example the 2-methoxyethyl, 2-ethoxyethyl, 2-butoxyethyl (butotyl), 2-butoxypropyl or 3-butoxypropyl ester. An example of a straight-chain or branched C₁-C₆-alkylthio ester is the ethylthio ester.

(1) Inhibition of HPPD (Hydroxyphenyl Pyruvate Dioxygenase): a substance that has herbicidal activity per se or a substance that is used in combination with other herbicides and/or additives which can change its effect, and the substance can act by inhibiting HPPD. Substances which are capable of producing herbicidal activity by inhibiting HPPD are well known in the art, including but not limited to the following types:

1) triketones, e.g., sulcotrione (CAS NO.: 99105-77-8), mesotrione (CAS NO.: 104206-82-8), bicyclopyrone (CAS NO.: 352010-68-5), tembotrione (CAS NO.: 335104-84-2), tefuryltrione (CAS NO.: 473278-76-1), benzobicyclon (CAS NO.: 156963-66-5);

2) diketonitriles, e.g., 2-cyano-3-cyclopropyl-1-(2-methylsulfonyl-4-trifluoromethylphenyl)propane-1,3-dione (CAS NO.: 143701-75-1), 2-cyano-3-cyclopropyl-1-(2-methylsulfonyl-3,4-dichlorophenyl)propane-1,3-dione (CAS NO.: 212829-55-5), 2-cyano-1-[4-(methylsulfonyl)-2-trifluoromethylphenyl]-3-(1-methyl cycloprop-yl)propane-1,3-dione (CAS NO.: 143659-52-3);

3) isoxazoles, e.g., isoxaflutole (CAS NO.: 141112-29-0), isoxachlortole (CAS NO.: 141112-06-3), clomazone (CAS NO.: 81777-89-1);

4) pyrazoles, e.g., topramezone (CAS NO.: 210631-68-8); pyrasulfotole (CAS NO.: 365400-11-9), pyrazoxyfen (CAS NO.: 71561-11-0); pyrazolate (CAS NO.: 58011-68-0), benzofenap (CAS NO.: 82692-44-2), bipyrazone (CAS NO.: 1622908-18-2), tolpyralate (CAS NO.: 1101132-67-5), fenpyrazone (CAS NO.: 1992017-55-6), cypyrafluone (CAS NO.: 1855929-45-1), tripyrasulfone (CAS NO.: 1911613-97-2);

5) benzophenons;

6) others: lancotrione (CAS NO.: 1486617-21-3), fenquinotrione (CAS NO.: 1342891-70-6), fufengcao'an (CAS NO:2421252-30-2);

and those mentioned in patent CN105264069A.

(2) Inhibition of EPSPS (Enolpyruvyl Shikimate Phosphate Synthase): e.g., sulphosate, Glyphosate, glyphosate-isopropylammonium, and glyphosate-trimesium.

(3) Inhibition of PPO (Protoporphyrinogen Oxidase) can be divided into pyrimidinediones, diphenyl-ethers, phenylpyrazoles, N-phenylphthalimides, thiadiazoles, oxadiazoles, triazolinones, oxazolidinedionesand other herbicides with different chemical structures.

In an exemplary embodiment, pyrimidinediones herbicides include but not limited to butafenacil (CAS NO: 134605-64-4), saflufenacil (CAS NO: 372137-35-4), benzfendizone (CAS NO: 158755-95-4), tiafenacil (CAS NO: 1220411-29-9), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (Epyrifenacil, CAS NO: 353292-31-6), 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS NO: 1304113-05-0), 3-[7-Chloro-5-fluoro-2-(trifluoromethyl)-1H-benzimidazol-4-yl]-1-methyl-6-(trifluoromethyl)-1H-pyrimidine-2,4-dione (CAS NO: 212754-02-4), flupropacil (CAS NO: 120890-70-2), uracil containing isoxazoline disclosed in CN105753853A (for example, the compound

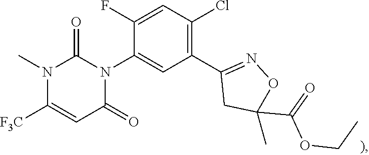 [SMILES: CCOC(=O)C1(C)CC(c2cc(-n3c(=O)cc(C(F)(F)F)n(C)c3=O)c(F)cc2Cl)=NO1]

uracil pyridines disclosed in WO2017/202768 and uracils disclosed in WO2018/019842;

Diphenyl-ethers herbicides include but not limited to fomesafen (CAS NO: 72178-02-0), oxyfluorfen (CAS NO: 42874-03-3), aclonifen (CAS NO: 74070-46-5), ethoxyfen-ethyl (CAS NO: 131086-42-5), lactofen (CAS NO: 77501-63-4), chlomethoxyfen (CAS NO: 32861-85-1), chlomitrofen (CAS NO: 1836-77-7), fluoroglycofen-ethyl (CAS NO: 77501-90-7), Acifluorfen or Acifluorfen sodium (CAS NO: 50594-66-6 or 62476-59-9), Bifenox (CAS NO: 42576-02-3), ethoxyfen (CAS NO: 188634-90-4), fluoronitrofen (CAS NO: 13738-63-1), furyloxyfen (CAS NO: 80020-41-3), nitrofluorfen (CAS NO: 42874-01-1), and halosafen (CAS NO: 77227-69-1);

Phenylpyrazoles herbicides include but not limited to pyraflufen-ethyl (CAS NO: 129630-19-9), and fluazolate (CAS NO: 174514-07-9);

N-phenylphthalimides herbicides include but not limited to flumioxazin (CAS NO: 103361-09-7), cinidonethyl (CAS NO: 142891-20-1), Flumipropyn (CAS NO: 84478-52-4), and flumiclorac-pentyl (CAS NO: 87546-18-7);

Thiadiazoles herbicides include but not limited tofluthiacet-methyl (CAS NO: 117337-19-6), fluthiacet (CAS NO: 149253-65-6), and thidiazimin (CAS NO: 123249-43-4);

Oxadiazoles herbicides include but not limited to Oxadiargyl (CAS NO: 39807-15-3), and Oxadiazon (CAS NO: 19666-30-9);

Triazolinones herbicides include but not limited to carfentrazone (CAS NO: 128621-72-7), carfentrazone-ethyl (CAS NO: 128639-02-1), sulfentrazone (CAS NO: 122836-35-5), azafenidin (CAS NO: 68049-83-2), and bencarbazone (CAS NO: 173980-17-1);

Oxazolidinediones herbicides include but not limited to pentoxazone (CAS NO: 110956-75-7);

Other herbicides include but not limited to pyraclonil (CAS NO: 158353-15-2), flufenpyr-ethyl (CAS NO: 188489-07-8), profluazol (CAS NO: 190314-43-3), trifludimoxazin (CAS NO: 1258836-72-4), N-ethyl-3-2,6-dichloro-4-trifluoromethylphenoxy)-5-methyl-1H-pyrazole-1-carboxamide (CAS NO: 452098-92-9), N-tetrahydrofurfuryl-3-(2,6-dichloro-4-trifluoromethylphenoxy)-5-methyl-1H-pyrazole-1-carboxamide (CAS NO: 915396-43-9), N-ethyl-3-(2-chloro-6-fluoro-4-trifluoromethylphenoxy)-5-methyl-1H-pyrazole-1-carboxamide (CAS NO: 452099-05-7), N-tetrahydrofurfuryl-3-(2-chloro-6-fluoro-4-trifluoromethylphenoxy)-5-methyl-1H-pyrazole-1-carboxamide (CAS NO: 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS NO: 451484-50-7), 2-(2,2,7-Trifluoro-3-oxo-4-prop-2-ynyl-3,4-dihydro-2H-benzo[1,4]oxazin-6-yl)-4,5,6,7-tetrahydro-isoindole-1,3-dione (CAS NO: 1300118-96-0), methyl (E)-4-[2-chloro-5-[4-chloro-5-(difluoromethoxy)-1H-methyl-pyrazol-3-yl]-4-fluoro-phenoxy]-3-methoxy-but-2-enoate (CAS NO: 948893-00-3), phenylpyridines disclosed in WO2016/120116, benzoxazinone derivatives disclosed in EP09163242.2, and compounds represented by general formula I

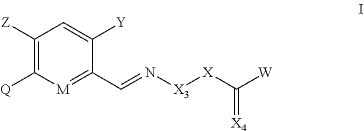

(See patent CN202011462769.7);

In another exemplary embodiment, Q represents

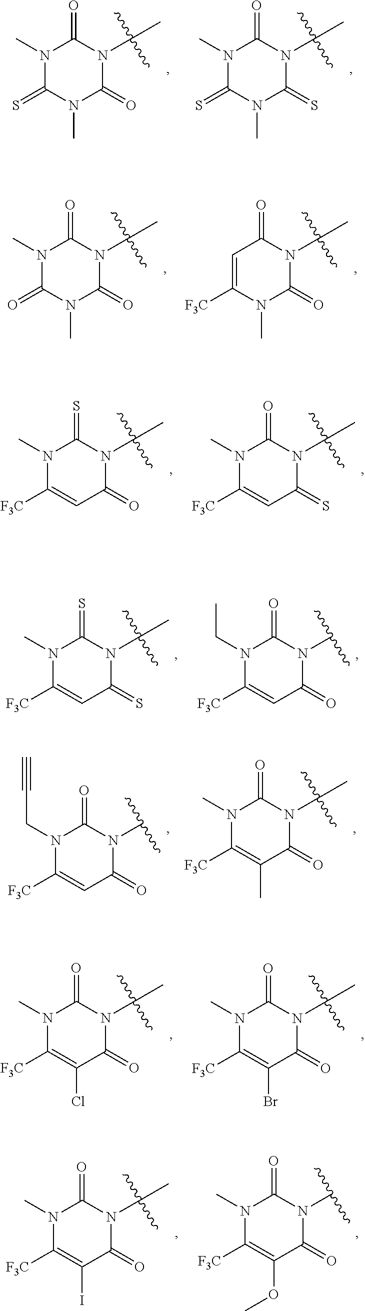

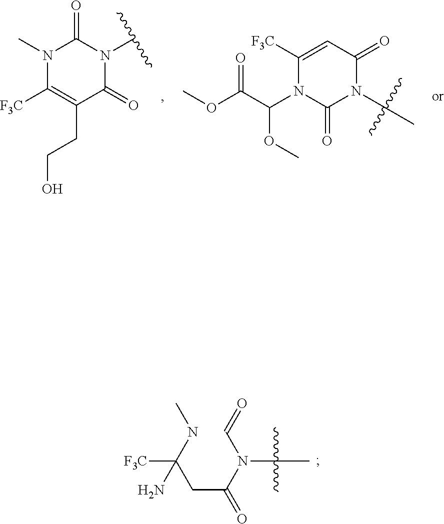 [SMILES: *n1c(=O)c(CCO)c(C(F)(F)F)n(C)c1=O ; *n1c(=O)cc(C(F)(F)F)n(C(OC)C(=O)OC)c1=O ; *N(C=O)C(=O)CC(N)(NC)C(F)(F)F]

Y represents halogen, halo C₁-C₆ alkyl or cyano;

Z represents halogen

M represents CH or N;

X represents —CX₁X₂—(C1-C6 alkyl)ₙ-, —(C1-C6 alkyl)-CX₁X₂—(C1-C6 alkyl)ₙ- or —(CH₂)ᵣ—; n represents 0 or 1; r represents an integer of 2 or more;

X₁, X₂ each independently represent H, halogen, C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, C3-C6 cycloalkyl C1-C6 alkyl, C1-C6 alkoxy, C1-C6 alkylthio, hydroxy C1-C6 alkyl, C1-C6 alkoxy C1-C6 alkyl, phenyl or benzyl;

X₃, X₄ each independently represent O or S;

W represents hydroxy, C1-C6 alkoxy, C2-C6 alkenyloxy, C2-C6 alkynyloxy, halo C1-C6 alkoxy, halo C2-C6 alkenyloxy, halo C2-C6 alkynyloxy, C3-C6 cycloalkyloxy, phenyloxy, sulfhydryl, C1-C6 alkylthio, C2-C6 alkenylthio, C2-C6 alkynylthio, halo C1-C6 alkylthio, halo C2-C6 alkenylthio, halo C2-C6 alkynylthio, C3-C6 cycloalkylthio, phenylthio, amino or C1-C6 alkylamino.

In another exemplary embodiment, the compound represented by the general formula I is selected from compound A: Q represents

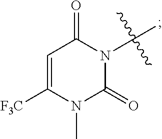

Y represents chlorine; Z represents fluorine; M represents CH; X represents—C*X₁X₂—(C1-C6 alkyl)ₙ-(C* is the chiral center, R configuration), n represents 0; X₁ represents hydrogen; X₂ represents methyl; X₃ and X₄ each independently represent O; W represents methoxy.

4) Inhibition of ALS (Acetolactate Synthase) including but not limited to the following herbicides or their mixtures:

(1) sulfonylureas such as amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, trifloxysulfuron-sodium, triflusulfuron, triflusulfuron-methyl and tritosulfuron;

(2) imidazolinones such as imazamethabenz, imazamethabenz-methyl, imazamox, imazapic, imazapyr, imazaquin and imazethapyr;

(3) triazolopyrimidine herbicides and sulfonanilides such as cloransulam, cloransulam-methyl, diclosulam, flumetsulam, florasulam, metosulam, penoxsulam, pyroxsulam, pyrimisulfan and triafamone;

(4) pyrimidinylbenzoates such as bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methylethyl ester (CAS NO.: 420138-41-6), 4-[[[2-[(4,6-dimethoxy-2-pyrimidinyl) oxy]phenyl]methyl]amino]-benzoic acid propyl ester (CAS NO.: 420138-40-5), N-(4-bromophenyl)-2-[(4,6-dimethoxy-2-pyrimidinyl)oxy]benzenemethanamine (CAS NO.: 420138-01-8);

(5) sulfonylaminocarbonyl-triazolinone herbicides such as flucarbazone, flucarbazone-sodium, propoxycarbazone, propoxycarbazone-sodium, thiencarbazone and thiencarbazone-methyl;

5) Inhibition of ACCase (Acetyl CoA Carboxylas): Fenthiaprop, alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS NO. 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS NO.: 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS NO.: 1033757-93-5); 4-(2′,4′-Dichloro-4-ethyl[1,1′-biphenyl]-3-yl)-2,2,6,6-tetramethyl-2H-pyran-3,5(4H,6H)-dione (CAS NO.: 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS NO.: 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro [1,1′-biphenyl]-3-yl)-3,6-dihydro-2,2,6,6-tetramethyl-2H-pyran-3-one (CAS NO.: 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS NO.: 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS NO.: 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro [1,1′-biphenyl]-3-yl)-5,6-dihydro-2,2,6,6-tetramethyl-5-oxo-2H-pyran-3-yl carbonic acid methyl ester (CAS NO.: 1312340-83-2); 4-(2′,4′-Dichloro-4-ethyl [1,1′-biphenyl]-3-yl)-5,6-dihydro-2,2,6,6-tetramethyl-5-oxo-2H-pyran-3-yl carbonic acid methyl ester (CAS NO.: 1033760-58-5).

(6) Inhibition of GS (Glutamine Synthetase): e.g., Bialaphos/bilanafos, Bilanaphos-natrium, Glufosinate-ammonium, Glufosinate, and glufosinate-P.

(7) Inhibition of PDS (Phytoene Desaturase): e.g., flurochloridone, flurtamone, beflubutamid, norflurazon, fluridone, Diflufenican, Picolinafen, and 4-(3-trifluoromethylphenoxy)-2-(4-trifluoromethylphenyl)pyrimidine (CAS NO: 180608-33-7).

(8) Inhibition of DHPS (Dihydropteroate Synthase): e.g., Asulam.

(9) Inhibition of DXPS (Deoxy-D-Xyulose Phosphate Synthase): e.g., Bixlozone, and Clomazone.

(10) Inhibition of HST (Homogentisate Solanesyltransferase): e.g., Cyclopyrimorate.

(11) Inhibition of SPS (Solanesyl Diphosphate Synthase): e.g., Aclonifen.

(12) Inhibition of Cellulose Synthesis: e.g., Indaziflam, Triaziflam, Chlorthiamid, Dichlobenil, Isoxaben, Flupoxam, 1-cyclohexyl-5-pentafluorphenyloxy-1⁴-[1,2,4,6]thiatriazin-3-ylamine (CAS NO: 175899-01-1), and the azines disclosed in CN109688807A.

(13) Inhibition of VLCFAS (Very Long-Chain Fatty Acid Synthesis) include but not limited to the following types:

1) α-Chloroacetamides: e.g., acetochlor, alachlor, butachlor, dimethachlor, dimethenamid, dimethenamid-P, metazachlor, metolachlor, metolachlor-S, pethoxamid, pretilachlor, propachlor, propisochlor, and thenylchlor;

2) α-Oxyacetamides: e.g., flufenacet, and mefenacet;

3) α-Thioacetamides: e.g., anilofos, and piperophos;

4) Azolyl-carboxamides: e.g., cafenstrole, fentrazamide, and ipfencarbazone;

5) Benzofuranes: e.g., Benfuresate, and Ethofumesate;

6) Isoxazolines: e.g., fenoxasulfone, and pyroxasulfone;

7) Oxiranes: e.g., Indanofan, and Tridiphane;

8) Thiocarbamates: e.g.,

Cycloate, Dimepiperate, EPTC, Esprocarb, Molinate, Orbencarb, Prosulfocarb, Thiobencarb/Benthiocarb, Tri-allate, Vernolate, and isoxazoline compounds of the formulae II.1, II.2, II.3, II.4, II.5, II.6, II.7, II.8 and II.9, and other isoxazoline compounds mentioned in patent WO 2006/024820, WO 2006/037945, WO 2007/071900, WO 2007/096576, etc.

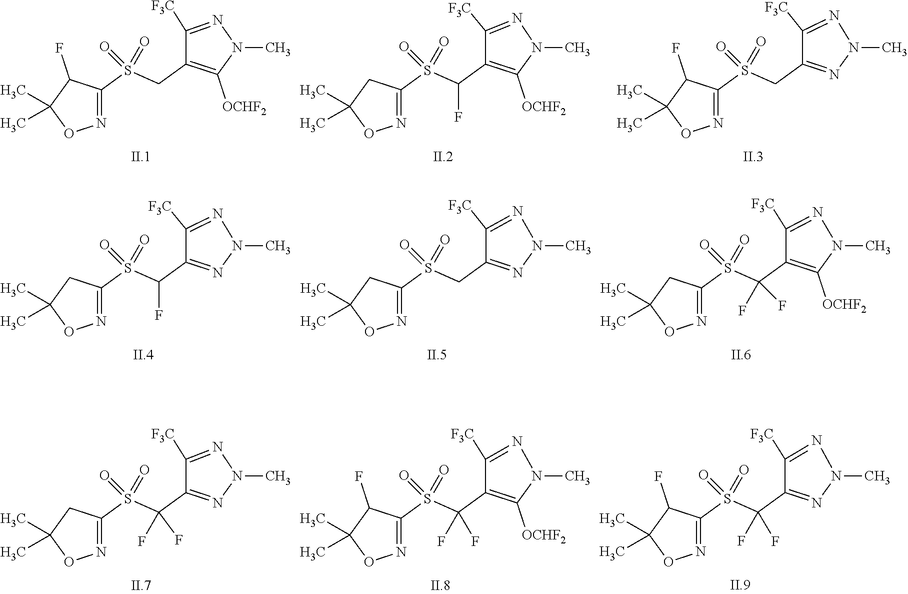

(14) Inhibition of fatty acid thioesterase: e.g., Cinmethylin, and Methiozolin;

(15) Inhibition of serine threonine protein phosphatase: e.g., Endothall.

(16) Inhibition of lycopene cyclase: e.g., Amitrole.

The term “wild-type” refers to a nucleic acid molecule or protein that can be found in nature.

In the present invention, the term “cultivation site” comprises a site where the plant of the present invention is cultivated, such as soil, and also comprises, for example, plant seeds, plant seedlings and grown plants. The term “weed-controlling effective amount” refers to an amount of herbicide that is sufficient to affect the growth or development of the target weed, for example, to prevent or inhibit the growth or development of the target weed, or to kill the weed. Advantageously, the weed-controlling effective amount does not significantly affect the growth and/or development of the plant seeds, plant seedlings or plants of the present invention. Those skilled in the art can determine such weed-controlling effective amount through routine experiments.

The term “gene” comprises a nucleic acid fragment expressing a functional molecule (such as, but not limited to, specific protein), including regulatory sequences before (5′ non-coding sequences) and after (3′ non-coding sequences) a coding sequence.

The DNA sequence that “encodes” a specific RNA is a DNA nucleic acid sequence that can be transcribed into RNA. The DNA polynucleotides can encode a RNA (mRNA) that can be translated into a protein, or the DNA polynucleotides can encode a RNA that cannot be translated into a protein (for example, tRNA, rRNA, or DNA-targeting RNA; which are also known as “non-coding” RNA or “ncRNA”).

The terms “polypeptide”, “peptide” and “protein” are used interchangeably in the present invention, and refer to a polymer of amino acid residues. The terms are applied to amino acid polymers in which one or more amino acid residues are artificially chemical analogs of corresponding and naturally occurring amino acids, as well as to naturally occurring amino acid polymers. The terms “polypeptide”, “peptide”, “amino acid sequence” and “protein” may also include their modification forms, including but not limited to glycosylation, lipid linkage, sulfation, γ-carboxylation of glutamic acid residue, hydroxylation and ADP-ribosylation.

The term “biologically active fragment” refers to a fragment that has one or more amino acid residues deleted from the N and/or C-terminus of a protein while still retaining its functional activity.

The terms “polynucleotide” and “nucleic acid” are used interchangeably and comprise DNA, RNA or hybrids thereof, which may be double-stranded or single-stranded.

The terms “nucleotide sequence” and “nucleic acid sequence” both refer to the sequence of bases in DNA or RNA.

Those of ordinary skill in the art can easily use known methods, such as directed evolution and point mutation methods, to mutate the DNA fragments as shown in SEQ ID No. 9 to SEQ ID No. 17 of the present invention. Those artificially modified nucleotide sequences that have at least 75% identity to any one of the foregoing sequences of the present invention and exhibit the same function are considered as derivatives of the nucleotide sequence of the present invention and equivalent to the sequences of the present invention.

The term “identity” refers to the sequence similarity to a natural nucleic acid sequence. Sequence identity can be evaluated by observation or computer software. Using a computer sequence alignment software, the identity between two or more sequences can be expressed as a percentage (%), which can be used to evaluate the identity between related sequences. “Partial sequence” means at least 5%, 10%, 20%, 30%, 40%, 50%, 60%, 70%, 80%, 90% or 95% of a given sequence.

The stringent condition may be as follows: hybridizing at 50° C. in a mixed solution of 7% sodium dodecyl sulfate (SDS), 0.5M NaPO₄, and 1 mM EDTA, and washing at 50° C. in 2×SSC and 0.1% SDS; or alternatively: hybridizing at 50° C. in a mixed solution of 7% SDS, 0.5M NaPO₄ and 1 mM EDTA, and washing at 50° C. in 1×SSC and 0.1% SDS; or alternatively: hybridizing at 50° C. in a mixed solution of 7% SDS, 0.5M NaPO₄ and 1 mM EDTA, and washing at 50° C. in 0.5×SSC and 0.1% SDS; or alternatively: hybridizing at 50° C. in a mixed solution of 7% SDS, 0.5M NaPO₄ and 1 mM EDTA, and washing at 50° C. in 0.1×SSC and 0.1% SDS; or alternatively: hybridizing at 50° C. in a mixed solution of 7% SDS, 0.5M NaPO₄ and 1 mM EDTA, and washing at 65° C. in 0.1×SSC and 0.1% SDS; or alternatively: hybridizing at 65° C. in a solution of 6×SSC, 0.5% SDS, and then membrane washing with 2×SSC, 0.1% SDS and 1×SSC, 0.1% SDS each once; or alternatively: hybridizing and membrane washing twice in a solution of 2×SSC, 0.1% SDS at 68° C., 5 min each time, and then hybridizing and membrane washing twice in a solution of 0.5×SSC, 0.1% SDS at 68° C., 15 min each time; or alternatively: hybridizing and membrane washing in a solution of 0.1×SSPE (or 0.1×SSC), 0.1% SDS at 65° C.

As used in the present invention, “expression cassette”, “expression vector” and “expression construct” refer to a vector such as a recombinant vector suitable for expression of a nucleotide sequence of interest in a plant. The term “expression” refers to the production of a functional product. For example, the expression of a nucleotide sequence may refer to the transcription of the nucleotide sequence (such as transcription to generate mRNA or functional RNA) and/or the translation of RNA into a precursor or mature protein.

The “expression construct” of the present invention can be a linear nucleic acid fragment, a circular plasmid, a viral vector, or, in some embodiments, can be an RNA (such as mRNA) that can be translated.

The “expression construct” of the present invention may comprise regulatory sequences and nucleotide sequences of interest from different sources, or regulatory sequences and nucleotide sequences of interest from the same source but arranged in a way different from those normally occurring in nature.

The “highly-expressing gene” in the present invention refers to a gene whose expression level is higher than that of a common gene in a specific tissue.

The terms “recombinant expression vector” or “DNA construct” are used interchangeably herein and refer to a DNA molecule comprising a vector and at least one insert. Recombinant expression vectors are usually produced for the purpose of expression and/or propagation of the insert or for the construction of other recombinant nucleotide sequences. The insert may be operably or may be inoperably linked to a promoter sequence and may be operably or may be inoperably linked to a DNA regulatory sequence.

The terms “regulatory sequence” and “regulatory element” can be used interchangeably and refer to a nucleotide sequence that is located at the upstream (5′ non-coding sequence), middle or downstream (3′ non-coding sequence) of a coding sequence, and affects the transcription, RNA processing, stability or translation of a related coding sequence. Plant expression regulatory elements refer to nucleotide sequences that can control the transcription, RNA processing or stability or translation of a nucleotide sequence of interest in plants.

The regulatory sequences may include, but are not limited to, promoters, translation leader sequences, introns, and polyA recognition sequences.

The term “promoter” refers to a nucleic acid fragment capable of controlling the transcription of another nucleic acid fragment. In some embodiments of the present invention, the promoter is a promoter capable of controlling gene transcription in plant cells, regardless of whether it is derived from plant cells. The promoter can be a constitutive promoter or a tissue-specific promoter or a developmentally regulated promoter or an inducible promoter.

The term “strong promoter” is a well-known and widely used term in the art. Many strong promoters are known in the art or can be identified by routine experiments. The activity of the strong promoter is higher than the activity of the promoter operatively linked to the nucleic acid molecule to be overexpressed in a wild-type organism, for example, a promoter with an activity higher than the promoter of an endogenous gene. Preferably, the activity of the strong promoter is higher by about 2%, 5%, 8%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000% or more than 1000% than the activity of the promoter operably linked to the nucleic acid molecule to be overexpressed in the wild-type organism. Those skilled in the art know how to measure the activity of a promoter and compare the activities of different promoters.

The term “constitutive promoter” refers to a promoter that will generally cause gene expression in most cell types in most cases. “Tissue-specific promoter” and “tissue-preferred promoter” are used interchangeably, and refer to a promoter that is mainly but not necessarily exclusively expressed in a tissue or organ, and also expressed in a specific cell or cell type. “Developmentally regulated promoter” refers to a promoter whose activity is determined by a developmental event. “Inducible promoter” responds to an endogenous or exogenous stimulus (environment, hormone, chemical signal, etc.) to selectively express an operably linked DNA sequence.

As used herein, the term “operably linked” refers to a connection of a regulatory element (for example, but not limited to, promoter sequence, transcription termination sequence, etc.) to a nucleic acid sequence (for example, a coding sequence or open reading frame) such that the transcription of the nucleotide sequence is controlled and regulated by the transcription regulatory element. The techniques for operably linking regulatory element region to nucleic acid molecule are known in the art.

The “introducing” a nucleic acid molecule (such as a plasmid, linear nucleic acid fragment, RNA, etc.) or protein into a plant refers to transforming a cell of the plant with the nucleic acid or protein so that the nucleic acid or protein can function in the plant cell. The term “transformation” used in the present invention comprises stable transformation and transient transformation.

The term “stable transformation” refers to that the introduction of an exogenous nucleotide sequence into a plant genome results in a stable inheritance of the exogenous gene. Once stably transformed, the exogenous nucleic acid sequence is stably integrated into the genome of the plant and any successive generations thereof.

The term “transient transformation” refers to that the introduction of a nucleic acid molecule or protein into a plant cell to perform function does not result in a stable inheritance of the foreign gene. In transient transformation, the exogenous nucleic acid sequence is not integrated into the genome of the plant.

Changing the expression of endogenous genes in organisms includes two aspects: intensity and spatial-temporal characteristics. The change of intensity includes the increase (knock-up), decrease (knock-down) and/or shut off the expression of the gene (knock-out); the spatial-temporal specificity includes temporal (growth and development stage) specificity and spatial (tissue) specificity, as well as inducibility. In addition, it includes changing the targeting of a protein, for example, changing the feature of cytoplasmic localization of a protein into a feature of chloroplast localization or nuclear localization.

Unless otherwise defined, all technical and scientific terms used herein have the same meanings as commonly understood by those of ordinary skill in the art to which the present invention pertains. Although any methods and materials similar or equivalent to those described herein can also be used in the practice or testing of the present invention, the preferred methods and materials are now described.

All publications and patents cited in this description are incorporated herein by reference as if each individual publication or patent is exactly and individually indicated to be incorporated by reference, and is incorporated herein by reference to disclose and describe methods and/or materials related to the publications cited. The citation of any publication which it was published before the filing date should not be interpreted as an admission that the present invention is not eligible to precede the publication of the existing invention. In addition, the publication date provided may be different from the actual publication date, which may require independent verification.

Unless specifically stated or implied, as used herein, the terms “a”, “a/an” and “the” mean “at least one.” All patents, patent applications, and publications mentioned or cited herein are incorporated herein by reference in their entirety, with the same degree of citation as if they were individually cited.

The present invention has the following advantageous technical effects:

The present invention comprehensively uses the information of the following two different professional fields to develop a method for directly creating new genes in organisms, completely changing the conventional use of the original gene editing tools (i.e., knocking out genes), realizing a new use thereof for creating new genes, in particular, realizing an editing method for knocking up endogenous genes by using gene editing technology to increase the expression of target genes. The first is the information in the field of gene editing, that is, when two or more different target sites and Cas9 simultaneously target the genome or organism, different situations such as deletion, inversion, doubling or inversion-doubling may occur. The second is the information in the field of genomics, that is, the information about location and distance of different genes in the genome, and specific locations, directions and functions of different elements (promoter, 5′UTR, coding region (CDS), different domain regions, terminator, etc.) in genes, and expression specificity of different genes, etc. By combining the information in these two different fields, breaks are induced at specific sites of two or more different genes or at two or more specific sites within a single gene (specific sites can be determined in the field of genomics), a new combination of different gene elements or functional domains can be formed through deletion, inversion, doubling, and inversion-doubling or chromosome arm exchange, etc. (the specific situations would be provided in the field of gene editing), thereby specifically creating a new gene in the organism.

The new genes created by the present invention are formed by the fusion or recombination of different elements of two or more genes under the action of the spontaneous DNA repair mechanism in the organism to change the expression intensity, spatial-temporal specificity, special functional domains and the like of the original gene without an exogenous transgene or synthetic gene elements. Because the new gene has the fusion of two or more different gene elements, this greatly expands the scope of gene mutation, and will produce more abundant and diverse functions, thus it has a wide range of application prospects. At the same time, these new genes are not linked to the gene editing vectors, so the vector elements can be removed through genetic segregation, and thereby resulting in non-transgenic biological materials containing the new genes for animal and plant breeding. Alternatively, non-integrated transient editing can be performed by delivery of mRNA or ribonucleic acid protein complex (RNP) to create non-genetically modified biological materials containing the new genes. This process is non-transgenic and the resultant edited materials would contain no transgene as well. In theory and in fact, these new genes can also be obtained through traditional breeding techniques (such as radiation or chemical mutagenesis). The difference is that the screening with traditional techniques requires the creation of libraries containing a huge number of random mutants and thus it is time-consuming and costly to screen new functional genes. While in the present invention, new functional genes can be created through bioinformatics analysis combined with gene editing technology, the breeding duration can be greatly shortened. The method of the present invention is not obliged to the current regulations on gene editing organisms in many countries.

In addition, the new gene creation technology of the present invention can be used to change many traits in organisms, including the growth, development, resistance, yield, etc., and has great application value. The new genes created may have new regulatory elements (such as promoters), which will change the expression intensity and and/or spatial-temporal characteristics of the original genes, or will have new amino acid sequences and thus have new functions. Taking crops as an example, changing the expression of specific genes can increase the resistance of crops to noxious organisms such as pests and weeds and abiotic stresses such as drought, waterlogging, and salinity, and can also increase yield and improve quality. Taking fish as an example, changing the expression characteristics of growth hormone in fish can significantly change its growth and development speed.

### BRIEF DESCRIPTION OF DRAWINGS

FIG. 1 shows a schematic diagram of creating a new HPPD gene in rice.

FIG. 2 shows a schematic diagram of creating a new EPSPS gene in rice.

FIG. 3 shows a schematic diagram of creating a new PPOX gene in Arabidopsis thaliana.

FIG. 4 shows a schematic diagram of creating a new PPOX gene in rice.

FIG. 5 shows the sequencing results for the HPPD-duplication Scheme tested with rice protoplast.

FIG. 6 shows the map of the Agrobacterium transformation vector pQY2091 for rice.

FIG. 7 shows the electrophoresis results of the PCR products for the detection of new gene fragments in pQY2091 transformed hygromycin resistant rice callus. The arrow indicates the PCR band of the new gene created by the fusion of the promoter of the UBI2 gene with the coding region of the HPPD. The numbers are the numbers of the different callus samples. M represents DNA Marker, and the band sizes are 100 bp, 250 bp, 500 bp, 750 bp, 1000 bp, 2000 bp, 2500 bp, 5000 bp, 7500 bp in order.

FIG. 8 shows the electrophoresis results of the PCR products for the detection of new gene fragments in pQY2091 transformed rice T0 seedlings. The arrow indicates the PCR band of the new gene created by the fusion of the promoter of the UBI2 gene with the coding region of the HPPD. The numbers are the serial numbers of the different T0 seedlings. M represents DNA Marker, and the band sizes are sequentially 100 bp, 250 bp, 500 bp, 750 bp, 1000 bp, 2000 bp, 2500 bp, 5000 bp, 7500 bp.

FIG. 9 shows the test results for the resistance to Bipyrazone of the QY2091 T0 generation of the HPPD gene doubling strain. In the same flowerpot, the wild-type Jinjing 818 is on the left, and the HPPD doubling strain is on the right.

FIG. 10 shows the relative expression levels of the HPPD and UBI2 genes in the QY2091 TO generation of the HPPD gene doubling strain. 818CK1 and 818CK3 represent two control plants of the wild-type Jinjing 818; 13M and 20M represent the primary tiller leaf samples of the QY2091-13 and the QY2091-20 T0 plants; 13 L and 20 L represent the secondary tiller leaf samples of the QY2091-13 and the QY2091-20 T0 plants used in the herbicide resistance test.

FIG. 11 shows a schematic diagram of the possible genotypes of QY2091 T1 generation and the binding sites of the molecular detection primers.

FIG. 12 shows the comparison of the sequencing results detecting the HPPD doubling and the predicted doubled sequences for QY2091-13 and QY2091-20.

FIG. 13 shows the results of the herbicide resistance test for the T1 generation of the QY2091 HPPD doubling strain at the seedling stage.

FIG. 14 shows a schematic diagram of the types of the possible editing event of rice PPO1 gene chromosome fragment inversion and the binding sites of molecular detection primers.

FIG. 15 shows the sequencing results of the EPSPS-inversion detection.

FIG. 16 shows the map of the rice Agrobacterium transformation vector pQY2234.

FIG. 17 shows the electrophoresis results of the PCR products for the detection of new gene fragments of hygromycin resistant rice callus transformed with pQY2234. The arrow indicates the PCR band of the new gene created by the fusion of the promoter of the CP12 gene with the coding region of the PPO1. The numbers are the serial numbers of different callus samples. M represents DNA Marker, and the band sizes are sequentially 100 bp, 250 bp, 500 bp, 750 bp, 1000 bp, 2000 bp, 2500 bp, 5000 bp, 7500 bp.

FIG. 18 shows the resistance test results of the PPO1 gene inversion strain to Compound A of the QY2234 T0 generation. Under the same treatment dose, the left flowerpot is the wild-type Huaidao No. 5 control, and the right is the PPO1 inversion strain.

FIG. 19 shows the relative expression levels of PPO1 and CP12 genes in the QY2234 TO generation PPO1 inversion strain. H5CK1 and H5CK2 represent two wild-type Huaidao No. 5 control plants; 252M, 304M and 329M represent the primary tiller leaf samples of QY2234-252, QY2234-304 and QY2234-329 T0 plants; 252 L, 304 L and 329 L represent secondary tiller leaf samples.

FIG. 20 shows the comparison of the sequencing result of the PPO1 inversion with the predicted inversion sequence in the Huaidao 5 background.

FIG. 21 shows the comparison of the sequencing result of the PPO1 inversion with the predicted inversion sequence in the Jinjing 818 background.

FIG. 22 shows the herbicide resistance test results for the T1 generation of the QY2234 PPO1 inversion strain at seedling stage.

FIG. 23 Duplication created a new GH1 gene cassette in zebrafish embryos. The GH1 gene is the growth hormone gene in zebrafish. Col1A1a is collagen type I alpha 1a gene. Col1A1a-GH1 fusion was the new gene cassette as a result of the duplication. DNA template used for PCR amplification in the Control group (CK) was extracted from young zebrafish without microinjection. DNA template used for PCR amplification in the Treatment group (RNP treat) was DNA sample extracted from young zebrafish after microinjection.

FIG. 24 PPO1 inversion event lines were tested for herbicide resistance in the field at T1 generation of QY2234 rice plants. WT is wild-type Jinjing 818. 5 # and 42 # represent samples from the PPO1 inversion event lines of QY2234/818-5 and QY2234/818-42, respectively. The herbicide tested was PPO inhibitor compound A.

FIG. 25 shows the Western Blot detection of PPO1 protein in the T1 rice plants of the QY2234 lines. 5 #, 42 #, 114 #, and 257 # represent the samples from the inversion event lines of QY2234/818-5, QY2234/818-42, QY2234/818-144, and QY2234/818-257, respectively.

FIG. 26 shows the field assay of HPPD inhibitor herbicide resistance under field conditions at T1 generation of QY2091 rice plants. 12 # and 21 # represent QY2091-12 and QY2091-21 duplication event lines, respectively. The herbicide tested was HHPD inhibitor Bipyrazone.

FIG. 27 A schematic diagram of the duplicated DNA fragment harboring PPO1 gene in rice, and 4 duplicated events were detected in rice protoplast cells using sequencing peak comparison. pQY2648, pQY2650, pQY2651, pQY2653 are the vector numbers tested. R2, F2 were used as sequencing primers. The diagram is not in proportion with DNA segment lengths.

FIG. 28 A schematic diagram of fragment translocation between chromosome1 and chromosome2 that up-regulates HPPD gene expression in rice. After targeted fragment translocation, CP12 gene promoter drives HPPD CDS expression; at the same time, HPPD gene promoter drives CP12 CDS expression. The diagram is not in proportion with DNA segment lengths.

FIG. 29 Fusion of the promoter of CP12 and the coding region of HPPD was detected in rice protoplast cells transformed with pQY2257. The diagram is not in proportion with DNA segment lengths.

FIG. 30 Fusion of the promoter of HPPD and the coding region of CP12 was detected in rice protoplast cells transformed with pQY2259. The diagram is not in proportion with DNA segment lengths.

FIG. 31 A schematic diagram of knocking-up HPPD gene expression as a result of the duplication of the segment between the two targeted cuts in rice, which was mediated by CRISPR/LbCpf1. The diagram is not in proportion with DNA segment lengths.

FIG. 32 A schematic diagram of fusing the OsCATC gene with a chloroplast signal peptide domain (LOC4331514CTP) through the deletion of the segment between the two targeted cuts in rice protoplast, which results in OsCATC protein have a chloroplast signal peptide domain and thus could go to chloroplast after expressed; and the positive events were detected in rice protoplast cells, which was demonstrated by sequencing. CTP stands for chloroplast signal peptide domain. The diagram is not in proportion with DNA segment lengths.

FIG. 33 A schematic diagram of the OsGLO3 gene linking the chloroplast signal peptide domain (LOC4337056CTP) through chromosome fragment inversion between the targeted cuts, which results in OsGLO3 protein have a chloroplast signal peptide domain and thus could go to chloroplast after expressed; while LOC4337056 gene drops its CTP; and the detection results of positive event rice protoplasts. CTP stands for chloroplast signal peptide domain. The diagram is not in proportion with DNA segment lengths.

FIG. 34 A schematic diagram showing knock-up of PPO2 gene by duplication of the DNA fragments between the two targeted cuts in rice. A new gene is produced where the SAMDC strong promoter drives the expression of PPO2. The diagram is not in proportion with DNA segment lengths.

FIG. 35 Positive duplication events were detected in pQY1386-transformed rice calli as indicated by alignment of sequencing data. 28 #, 62 # are two duplication-positive calli. The diagram is not in proportion with DNA segment lengths.

FIG. 36 Positive duplication events were detected in pQY1387-transformed rice calli as indicated by alignment of sequencing data. 64 #, 82 #, 110 #, 145 # are duplication-positive calli. The diagram is not in proportion with DNA segment lengths.

FIG. 37 Positive duplication events were detected in T0 rice plants (QY1387/818-2) emerged from pQY1387-transformed calli. The repair outcomes of two targets as well as the duplication joint were aligned with Sanger sequencing data. The diagram is not in proportion with DNA segment lengths.

FIG. 38 The detection results of the relative expression level of PPO2 in QY1387/818 T0 rice plants. As expected, PPO2 expression significantly increased meanwhile SAMDC expression significantly reduced.

FIG. 39 Herbicide resistance assay of rice QY1387 T0 plants. 2 # represents the 1387/818-2 line, 4 # represents the 1387/818-4 line, and WT is the wild type of Jinjing 818. The herbicide tested is PPO inhibitor compound A

FIG. 40 A schematic diagram of creation of new PPO2 genes by DNA fragment inversion between the two targeted cuts in rice. The diagram is not in proportion with DNA segment lengths.

FIG. 41 Positive inversion events were detected in QY2611-transformed rice calli as indicated by alignment of sequencing data. 10 # represents the QY2611/818-10 callus, 13 # represents the QY2611/818-13 callus. The diagram is not in proportion with DNA segment lengths.

FIG. 42 Positive inversion events were detected in QY2612-transformed rice calli as indicated by alignment of sequencing data. 5 # represents the QY2612/818-5 callus, 34 # represents the QY2612/818-34 callus. The diagram is not in proportion with DNA segment lengths.

FIG. 43 A schematic diagram of successful generation of new PPO2 gene cassette in maize protoplast cells, through duplication of the segment between the two targeted cuts and demonstrated by alignment of Sanger sequencing data. pQY1340 and pQY1341 are test vectors. The diagram is not in proportion with DNA segment lengths.

FIG. 44 A schematic diagram of successful generation of new PPO2-2A gene cassette in wheat protoplast cells transfected with pQY2626 vector, through inversion of the segment between the two targeted cuts, and demonstrated by alignment of Sanger sequencing data. The diagram is not in proportion with DNA segment lengths.

FIG. 45 A schematic diagram of successful generation of new PPO2-2B gene cassette in wheat protoplast cells transfected with pQY2631 vector, through duplication of the segment between the two targeted cuts, and demonstrated by alignment of Sanger sequencing data. The diagram is not in proportion with DNA segment lengths.

FIG. 46 A schematic diagram of successful generation of new PPO2-2D gene cassette in wheat protoplast cells transfected with pQY2635 vector, through duplication of the segment between the two targeted cuts, and demonstrated by alignment of Sanger sequencing data. The diagram is not in proportion with DNA segment lengths.

FIG. 47 Sequencing results of chromosome fragment inverted rice Line QY1085/818-23.

FIG. 48 Sequencing results of chromosome fragment duplicated rice Line QY1089/818-321.

FIG. 49 A schematic diagram of successful generation of new IGF2 (Insulin-like growth factor 2) gene cassette driven by TNNI2 gene promoter, through inversion of the segment between the two targeted cuts, and demonstrated the detection of the positive fusion event of Pig TNNI2 promoter and IGF2 gene in pig primary fibroblast cells. The diagram is not in proportion with DNA segment lengths.

FIG. 50 A schematic diagram of successful generation of new TNNI3 (muscle troponin T) gene cassette driven by IGF2 gene promoter, through inversion of the segment between the two targeted cuts, and demonstrated the detection of the fusion event of Pig IGF2 promoter and TNNT3 gene in pig primary fibroblast cells. The diagram is not in proportion with DNA segment lengths.

FIG. 51 shows the sequencing result of forward and reverse primers. The experiment result shows that the fragments between gh1 gene and col1a1a gene in zebra fish embryo are doubled.

FIG. 52 is the sequencing result. The experiment result shows that the coding area and the coding area & promotor of ddx5 gene and the coding area & the promotor of gh1 gene are exchanged due to the inversion of chromosome fragments;

FIG. 53 is the comparison diagram of inversion and wild type zebra fish. The result shows that the growth of zebra fish with upregulated expression is obviously accelerated.

FIG. 54 is a schematic diagram of Ubi2 promoter translocation to knock-up PPO2 gene in rice.

FIG. 55 shows the herbicide resistance test results for the T1 generation of the QY378-16 translocation rice at seedling stage.

### SPECIFIC MODELS FOR CARRYING OUT THE INVENTION

The present invention is further described in conjunction with the examples as follows. The following description is just illustrative, and the protection scope of the present invention should not be limited to this.

### Example 1: An Editing Method for Knocking Up the Expression of the Endogenous HPPD Gene by Inducing Doubling of Chromosome Fragment in Plant—Rice Protoplast Test

HPPD was a key enzyme in the pathway of chlorophyll synthesis in plants, and the inhibition of the activity of the HPPD would eventually lead to albino chlorosis and death of plants. Many herbicides, such as mesotrione and topramezone, were inhibitors with the HPPD as the target protein, and thus increasing the expression level of the endogenous HPPD gene in plants could improve the tolerance of the plants to these herbicides. The rice HPPD gene (as shown in SEQ ID NO: 6, in which 1-1067 bp is the promoter, and the rest is the expression region) locates on rice chromosome 2. Through bioinformatic analysis, it was found that rice Ubiquitin2 (hereinafter referred to as UBI2) gene (as shown in SEQ ID NO: 5, in which 1-2107 bp was the promoter, and the rest was the expression region) locates about 338 kb downstream of HPPD gene, and the UBI2 gene and the HPPD gene were in the same direction on the chromosome. According to the rice gene expression profile data provided by the International Rice Genome Sequencing Project (http://rice.plantbiology.msu.edu/index.shtml), the expression intensity of the UBI2 gene in rice leaves was 3 to 10 times higher than that of the HPPD gene, and the UBI2 gene promoter was a strong constitutively expressed promoter.

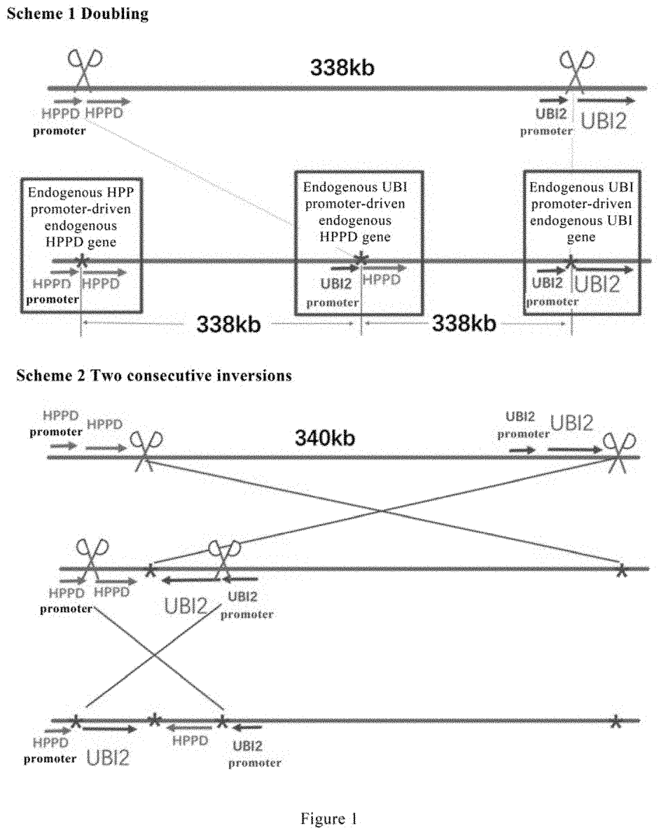

As shown in FIG. 1, Scheme 1 shows that double-strand breaks were simultaneously generated at the sites between the promoters and the CDS region of the HPPD and UBI2 genes respectively, the event of doubling the region between the two breaks were obtained after screening and identification, and a new gene could be formed by fusing the promoter of UBI2 and the coding region of HPPD together. In addition, according to Scheme 2 as shown in FIG. 1, a new gene in which the promoter of UBI2 and the coding region of HPPD were fused could also be formed by two consecutive inversions. First, the schemes as shown in FIG. 1 were tested in the rice protoplast system as follows:

1. Firstly, the genomic DNA sequences of the rice HPPD and UBI2 genes were input into the CRISPOR online tool (http://crispor.tefor.net/) to search for available editing target sites. After online scoring, the following target sites between the promoters and the CDS regions of HPPD and UBI2 genes were selected for testing:

| | |
|---|---|
| OsHPPD-guide RNA1 | GTGCTGGTTGCCTTGGCTGC |
| OsHPPD-guide RNA2 | CACAAATTCACCAGCAGCCA |
| OsHPPD-guide RNA3 | TAAGAACTAGCACAAGATTA |
| OsHPPD-guide RNA4 | GAAATAATCACCAAACAGAT |

The guide RNA1 and guide RNA2 located between the promoter and the CDS region of the HPPD gene, close to the start codon of the HPPD protein, and the guide RNA3 and guide RNA4 located between the promoter and CDS region of the UBI2 gene, close to the UBI2 protein initiation codon.

pHUE411 vector (https://www.addgene.org/62203/) is used as the backbone, and the following primers were designed for the above-mentioned target sites to perform vector construction as described in “Xing H L, Dong L, Wang Z P, Zhang H Y, Han C Y, Liu B, Wang X C, Chen Q J. A CRISPR/Cas9 Toolkit for multiplex genome editing in plants. BMC Plant Biol. 2014 Nov. 29; 14(1): 327”.

| Primer No. | DNA sequence (5′ to 3′) |
|---|---|
| OsHPPD-sgRN | ATATGGTCTCGGGCGGTGCTGGTTGCCTTGGCTGCGTTTTAGAGC |
| A1-F | TAGAAATAGCAAG |
| OsHPPD-sgRN | ATATGGTCTCGGGCGCACAAATTCACCAGCAGCCAGTTTTAGAG |
| A2-F | CTAGAAATAGCAAG |
| OsHPPD-sgRN | TATTGGTCTCTAAACTAATCTTGTGCTAGTTCTTAGCTTCTTGGT |
| A3-R | GCCGCGC |
| OsHPPD-sgRN | TATTGGTCTCTAAACATCTGTTTGGTGATTATTTCGCTTCTTGGTG |
| A4-R | CCGCGC |

gene editing vectors for the following dual-target combination were constructed following the method provided in the above-mentioned literature. Specifically, with the pCBC-MT1T2 plasmid (https://www.addgene.org/50593/) as the template, sgRNA1+3, sgRNA1+4, sgRNA2+3 and sgRNA2+4 double target fragments were amplified respectively for constructing the sgRNA expression cassettes. The vectorbackbone of pHUE411 was digested with BsaI, and recovered from the gel, and the target fragment was digested and directly used for the ligation reaction. T4 DNA ligase was used to ligate the vector backbone and the target fragment, and the ligation product was transformed into Trans5α competent cells. Different monoclones were picked and sequenced The Sparkjade High Purity Plasmid Mini Extraction Kit was used to extract plasmids from the clones with correct sequences, thereby obtaining recombinant plasmids, respectively named as pQY002065, pQY002066, pQY002067, and pQY002068, as follows:

pQY002065 pHUE411-HPPD-sgRNA1+3 combination of OsHPPD-guide RNA1, guide RNA3

pQY002066 pHUE411-HPPD-sgRNA1+4 combination of OsHPPD-guide RNA1, guide RNA4

pQY002067 pHUE411-HPPD-sgRNA2+3 combination of OsHPPD-guide RNA2, guide RNA3

pQY002068 pHUE411-HPPD-sgRNA2+4 combination of OsHPPD-guide RNA2, guide RNA4

2. Plasmids of high-purity and high-concentration were prepared for the above-mentioned pQY002065-002068 vectors as follows:

Plasmids were extracted with the Promega Medium Plasmid Extraction Kit (Midipreps DNA Purification System, Promega, A7640) according to the instructions. The specific steps were:

(1) Adding 5 ml of Escherichia coli to 300 ml of liquid LB medium containing kanamycin, and shaking at 200 rpm, 37° C. for 12 to 16 hours;

(2) Placing the above bacteria solution in a 500 ml centrifuge tube, and centrifuging at 5,000 g for 10 minutes, discarding the supernatant;

(3) Adding 3 ml of Cell Resuspension Solution (CRS) to resuspend the cell pellet and vortexing for thorough mixing;

(4) Adding 3 ml of Cell Lysis Solution (CLS) and mixing up and down slowly for no more than 5 minutes;

(5) Adding 3 ml of Neutralization Solution and mixed well by overturning until the color become clear and transparent;

(6) Centrifuging at 14,000 g for 15 minutes, and further centrifuging for 15 minutes if precipitate was not formed compact;

(7) Transferring the supernatant to a new 50 ml centrifuge tube, avoiding to suck in white precipitate into the centrifuge tube;

(8) Adding 10 ml of DNA purification resin (Purification Resin, shaken vigorously before use) and mixing well;

(9) Pouring the Resin/DNA mixture was poured into a filter column, and treating by the vacuum pump negative pressure method (0.05 MPa);

(10) Adding 15 ml of Column Wash Solution (CWS) to the filter column, and vacuuming.

(11) Adding 15 ml of CWS, and repeating vacuuming once; vacuuming was extended for 30 s after the whole solution passed through the filter column;

(12) Cutting off the filter column, transferring to a 1.5 ml centrifuge tube, centrifuging at 12,000 g for 2 minutes, removing residual liquid, and transferring the filter column to a new 1.5 ml centrifuge tube;

(13) Adding 200 μL of sterilized water preheated to 70° C., and keeping rest for 2 minutes;

(14) Centrifuging at 12,000 g for 2 minutes to elute the plasmid DNA; and the concentration was generally about 1 μg/μL.

3. Preparing Rice Protoplasts and Performing PEG-Mediated Transformation:

First, rice seedlings for protoplasts were prepared, which is of the variety Nipponbare. The seeds were provided by the Weeds Department of the School of Plant Protection, China Agricultural University, and expanded in house. The rice seeds were hulled first, and the hulled seeds were rinsed with 75% ethanol for 1 minute, treated with 5% (v/v) sodium hypochlorite for 20 minutes, then washed with sterile water for more than 5 times. After blow-drying in an ultra-clean table, they were placed in a tissue culture bottle containing ½ MS medium, 20 seeds for each bottle. Protoplasts were prepared by incubating at 26° C. for about 10 days with 12 hours light.

The methods for rice protoplast preparation and PEG-mediated transformation were conducted according to “Lin et al., 2018 Application of protoplast technology to CRISPR/Cas9 mutagenesis: from single-cell mutation detection to mutant plant regeneration. Plant Biotechnology Journal https://doi.org/10.1111/pbi.12870”. The steps were as follows:

(1) the leaf sheath of the seedlings was selected, cut into pieces of about 1 mm with a sharp Geely razor blade, and placed in 0.6 M mannitol and MES culture medium (formulation: 0.6 M mannitol, 0.4 M MES, pH 5.7) for later use. All materials were cut and transferred to 20 ml of enzymatic hydrolysis solution (formulation: 1.5% Cellulase R10/RS (YaKult Honsha), 0.5% Mecerozyme R10 (YaKult Honsha), 0.5M mannitol, 20 mM KCl, 20 mM MES, pH 5.7, 10 mM CaCl₂), 0.1% BSA, 5 mM β-mercaptoethanol), wrapped in tin foil and placed in a 28° C. shaker, enzymatically hydrolyzed at 50 rpm in the dark for about 4 hours, and the speed was increased to 100 rpm in the last 2 minutes;

(2) after the enzymatic lysis, an equal volume of W5 solution (formulation: 154 mM NaCl, 125 mM CaCl₂), 5 mM KCl, 15 mM MES) was added, shaken horizontally for 10 seconds to release the protoplasts. The cells after enzymatic lysis were filtered through a 300-mesh sieve and centrifuged at 150 g for 5 minutes to collect protoplasts;

(3) the cells were rinsed twice with the W5 solution, and the protoplasts were collected by centrifugation at 150 g for 5 minutes;

(4) the protoplasts were resuspended with an appropriate amount of MMG solution (formulation: 3.05 g/L MgCl₂, 1 g/L MES, 91.2 g/L mannitol), and the concentration of the protoplasts was about 2×10⁶ cells/mL.

The transformation of protoplasts was carried out as follows:

(1) to 200 μL of the aforementioned MMG resuspended protoplasts, endotoxin-free plasmid DNA of high quality (10-20 μg) was added and tapped to mix well;

(2) an equal volume of 40% (w/v) PEG solution (formulation: 40% (w/v) PEG, 0.5M mannitol, 100 mM CaCl₂)) was added, tapped to mix well, and kept rest at 28° C. in the dark for 15 minutes;

(3) after the induction of transformation, 1.5 ml of W5 solution was added slowly, tapped to mix the cells well. The cells were collected by centrifugation at 150 g for 3 minutes. This step was repeated once;

(4) 1.5 ml of W5 solution was added to resuspend the cells, and placed in a 28° C. incubator and cultured in the dark for 12-16 hours. For extracting protoplast genomic DNA, the cultivation should be carried out for 48-60 hours.

4. Genome Targeting and Detecting New Gene:

(1) First, protoplast DNAs were extracted by the CTAB method with some modifications. The specific method was as follows: the protoplasts were centrifuged, then the supernatant was discarded. 500 μL of DNA extracting solution (formulation: CTAB 20 g/L, NaCl 81.82 g/L, 100 mM Tris-HCl (pH 8.0), 20 mM EDTA, 0.2% β-mercaptoethanol) was added, shaken to mix well, and incubated in a 65° C. water bath for 1 hour; when the incubated sample was cooled, 500 μL of chloroform was added and mixed upside down and centrifuged at 10,000 rpm for 10 minutes; 400 μL of the supernatant was transferred to a new 1.5 ml centrifuge tube, 1 ml of 70% (v/v) ethanol was added and the mixture was kept at −20° C. for precipitating for 20 minutes; the mixture was centrifuged at 12,000 rpm for 15 minutes to precipitate the DNA; after the precipitate was air dried, 50 μL of ultrapure water was added and stored at −20° C. for later use.

(2) The detection primers in the following table were used to amplify the fragments containing the target sites on both sides or the predicted fragments resulting from the fusion of the UBI2 promoter and the HPPD coding region. The lengths of the PCR products were between 300-1000 bp, in which the primer8-F+primer6-R combination was used to detect the fusion fragment at the middle joint after the doubling of the chromosome fragment, and the product length was expected to be 630 bp.

| Primer | Sequence (5′ to 3′) |
|---|---|
| OsHPPDduplicated- | CACTACCATCCATCCATTTGC |
| primer1-F | |
| OsHPPDduplicated- | GAGTTCCCCGTGGAGAGGT |
| primer6-R | |
| OsHPPDduplicated- | TCCATTACTACTCTCCCCGATT |
| primer3-F | |
| OsHPPDduplicated- | GTGTGGGGGAGTGGATGAC |
| primer7-R | |
| OsHPPDduplicated- | TGTAGCTTGTGCGTTTCGAT |
| primer5-F | |
| OsHPPDduplicated- | GGGATGCCCTCTTTGTCC |
| primer2-R | |
| OsHPPDduplicated- | TCTGTGTGAAGATTATTGCCACT |
| primer8-F | |
| OsHPPDduplicated- | GGGATGCCCTCCTTATCTTG |
| primer4-R | |

The PCR reaction system was as follows:

| Components | Volume |
|---|---|
| 2 × 15 buffer solution | 5 μL |
| Forward primer (10 μM) | 2 μL |
| Reverse primer (10 μM) | 2 μL |
| Template DNA | 2 μL |
| Ultrapure water | Added to 50 μL |

(3) A PCR Reaction was Conducted Under the Following General Reaction Conditions:

| Step | Temperature | Time |
|---|---|---|
| Denaturation | 98° C. | 30 s |
| | 98° C. | 15 s |
| Amplification for | 58° C. | 15 s |
| 30-35 cycles | 72° C. | 30 s |
| Final extension | 72° C. | 3 min |
| Finished | 16° C. | 5 min |

(4) The PCR reaction products were detected by 1% agarose gel electrophoresis. The results showed that the 630 bp positive band for the predicted fusion fragment of the UBI2 promoter and the HPPD coding region could be detected in the pQY002066 and pQY002068 transformed samples.

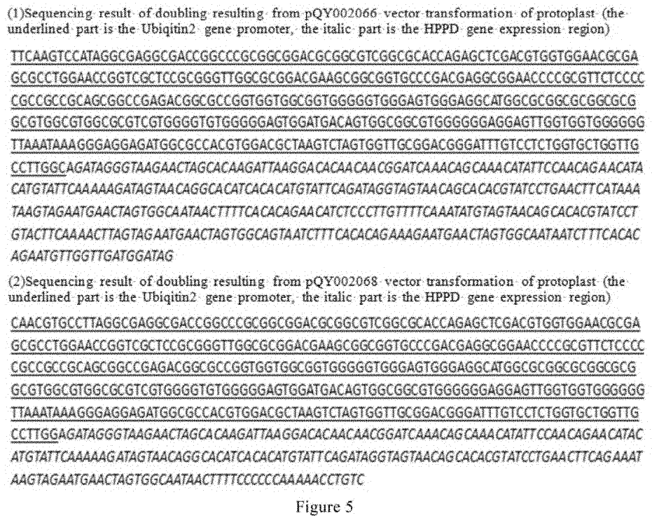

5. The positive samples of the fusion fragment of the UBI2 promoter and the HPPD coding region were sequenced for verification, and the OsHPPDduplicated-primer8-F and OsHPPDduplicated-primer6-R primers were used to sequence from both ends. As shown in FIG. 5, the promoter of the UBI2 gene and the expression region of the HPPD gene could be directly ligated, and the editing event of the fusion of the promoter of rice UBI2 gene and the expression region of the HPPD gene could be detected in the protoplast genomic DNA of the rice transformed with pQY002066 and pQY002068 plasmids, indicating that the scheme of doubling the chromosome fragments to form a new HPPD gene was feasible, a new HPPD gene which expression was driven by a strong promoter could be created, and this was defined as an HPPD doubling event. The sequencing result of the pQY002066 vector transformed protoplast for testing HPPD doubling event was shown in SEQ ID NO: 9; and the sequencing result of the pQY002068 vector transformed protoplast for testing HPPD doubling event was shown in SEQ ID NO: 10.

### Example 2: Creation of Herbicide-Resistant Rice with Knock-Up Expression of Endogenous HPPD Gene by Chromosome Fragment Doubling Through Agrobacterium-Mediated Transformation

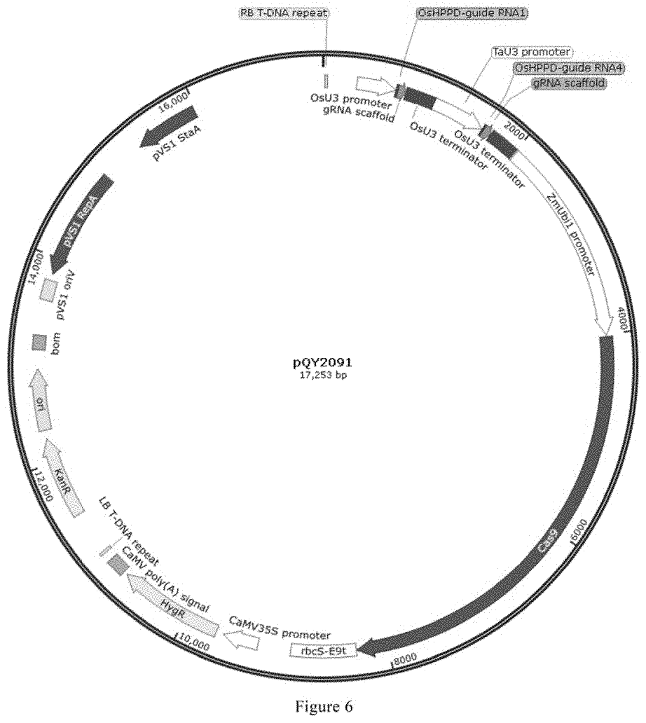

1. Construction of knock-up editing vector: Based on the results of the protoplast test in Example 1, the dual-target combination OsHPPD-guide RNA1: 5′GTGCTGGTTGCCTTGGCTGC3′ and OsHPPD-guide RNA4: 5′GAAATAATCACCAAACAGAT3′ with a high editing efficiency was selected. The Agrobacterium transformation vector pQY2091 was constructed according to Example 1. pHUE411 was used as the vector backbone and subjected to rice codon optimization. The map of the vector was shown in FIG. 6.

2. Agrobacterium Transformation of Rice Callus:

1) Agrobacterium transformation: 1 μg of the rice knock-up editing vector pQY2091 plasmid was added to 10 μl of Agrobacterium EHA105 heat-shock competent cells (Angyu Biotech, Catalog No. G6040), placed on ice for 5 minutes, immersed in liquid nitrogen for quick freezing for 5 minutes, then removed and heated at 37° C. for 5 minutes, and finally placed on ice for 5 minutes. 500 μl of YEB liquid medium (formulation: yeast extract 1 g/L, peptone 5 g/L, beef extract 5 g/L, sucrose 5 g/L, magnesium sulfate 0.5 g/L) was added. The mixture was placed in a shaker and incubated at 28° C., 200 rpm for 2˜3 hours; the bacteria were collected by centrifugation at 3500 rpm for 30 seconds, the collected bacteria were spread on YEB (kanamycin 50 mg/L+rifampicin 25 mg/L) plate, and incubated for 2 days in an incubator at 28° C.; the single colonies were picked and placed into liquid culture medium, and the bacteria were stored at −80° C.

2) Cultivation of Agrobacterium: The single colonies of the transformed Agrobacterium on the YEB plate was picked, added into 20 ml of YEB liquid medium (kanamycin 50 mg/L+rifampicin 25 mg/L), and cultured while stirring at 28° C. until the OD600 was 0.5, then the bacteria cells were collected by centrifugation at 5000 rpm for 10 minutes, 20-40 ml of AAM (Solarbio, lot number LA8580) liquid medium was added to resuspend the bacterial cells to reach OD600 of 0.2-0.3, and then acetosyringone (Solarbio, article number A8110) was added to reach the final concentration of 200 μM for infecting the callus.

3) Induction of rice callus: The varieties of the transformation recipient rice were Huaidao 5 and Jinjing 818, purchased from the seed market in Huai'an, Jiangsu, and expanded in house. 800-2000 clean rice seeds were hulled, then washed with sterile water until the water was clear after washing. Then the seeds were disinfected with 70% alcohol for 30 seconds, then 30 ml of 5% sodium hypochlorite was added and the mixture was placed on a horizontal shaker and shaken at 50 rpm for 20 minutes, then washed with sterile water for 5 times. The seeds were placed on sterile absorbent paper, air-dried to remove the water on the surface of the seeds, inoculated on an induction medium and cultivated at 28° C. to obtain callus.

The formulation of the induction medium: 4.1 g/L N6 powder+0.3 g/L hydrolyzed casein+2.878 g/L proline+2 mg/L 2,4-D+3% sucrose+0.1 g/L inositol+0.5 g glutamine+0.45% phytagel, pH 5.8.

4) Infection of rice callus with Agrobacterium: The callus of Huaidao No. 5 or Jinjing 818 subcultured for 10 days with a diameter of 3 mm was selected and collected into a 50 ml centrifuge tube; the resuspension solution of the Agrobacterium AAM with the OD600 adjusted to 0.2-0.3 was poured into the centrifuge tube containing the callus, placed in a shaker at 28° C. at a speed of 200 rpm to perform infection for 20 minutes; when the infection was completed, the bacteria solution was discarded, the callus was placed on sterile filter paper and air-dried for about 20 minutes, then placed on a plate containing co-cultivation medium to perform co-cultivation, on which the plate was covered with a sterile filter paper soaked with AAM liquid medium containing 100 μM acetosyringone; after 3 days of co-cultivation, the Agrobacterium was removed by washing (firstly washing with sterile water for 5 times, then washing with 500 mg/L cephalosporin antibiotic for 20 minutes), and selective cultured on 50 mg/L hygromycin selection medium.

The formulation of the co-cultivation medium: 4.1 g/L N6 powder+0.3 g/L hydrolyzed casein+0.5 g/L proline+2 mg/L 2,4-D+200 μM AS+10 g/L glucose+3% Sucrose+0.45% phytagel, pH 5.5.

3. Molecular Identification and Differentiation into Seedlings of Hygromycin Resistant Callus:

Different from the selection process of conventional rice transformation, with specific primers of the fusion fragments generated after the chromosome fragment doubling, hygromycin resistant callus could be molecularly identified during the callus selection and culture stage in the present invention, positive doubling events could be determined, and callus containing new genes resulting from fusion of different gene elements was selected for differentiation cultivation and induced to emerge seedlings. The specific steps were as follows:

1) The co-cultured callus was transferred to the selection medium for the first round of selection (2 weeks). The formulation of the selection medium is: 4.1 g/L N6 powder+0.3 g/L hydrolyzed casein+2.878 g/L proline+2 mg/L 2,4-D+3% sucrose+0.5 g glutamine+30 mg/L hygromycin (HYG)+500 mg/L cephalosporin (cef)+0.1 g/L inositol+0.45% phytagel, pH 5.8.

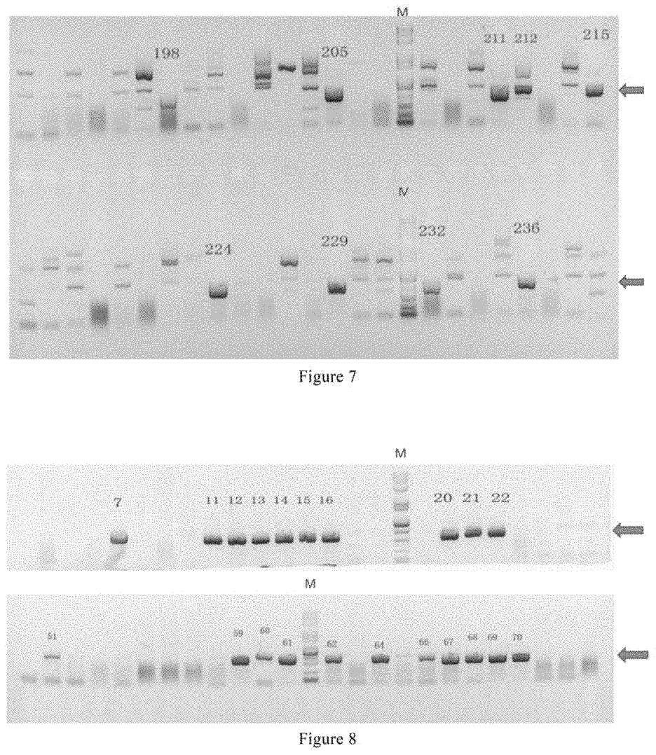

2) After the first round of selection was completed, the newly grown callus was transferred into a new selection medium for the second round of selection (2 weeks). At this stage, the newly grown callus with a diameter greater than 3 mm was clamped by tweezers to take a small amount of sample, the DNA thereof was extracted with the CTAB method described in Example 1 for the first round of molecular identification. In this example, the primer pair of OsHPPDduplicated-primer8-F (8F) and OsHPPD duplicated-primer6-R (6R) was selected to perform PCR identification for the callus transformed with the pQY2091 vector, in which the reaction system and reaction conditions were similar to those of Example 1. Among the total of 350 calli tested, no positive sample was detected in the calliof Huaidao 5, while 28 positive samples were detected in the calli of Jinjing 818. The PCR detection results of some calli were shown in FIG. 7.

3) The calli identified as positive by PCR were transferred to a new selection medium for the third round of selection and expanding cultivation; after the diameter of the calli was greater than 5 mm, the callus in the expanding cultivation was subjected to the second round of molecular identification using 8F+6R primer pair, the yellow-white callus at good growth status that was identified as positive in the second round was transferred to a differentiation medium to perform differentiation, and the seedlings of about 1 cm could be obtained after 3 to 4 weeks; the differentiated seedlings were transferred to a rooting medium for rooting cultivation; after the seedlings of the rooting cultivation were subjected to hardening off, they were transferred to a flowerpot with soil and placed in a greenhouse for cultivation. The formulation of the differentiation medium is: 4.42 g/L MS powder+0.5 g/L hydrolyzed casein+0.2 mg/L NAA+2 mg/L KT+3% sucrose+3% sorbitol+30 mg/L hygromycin+0.1 g/L inositol+0.45% phytagel, pH 5.8. The formulation of the rooting medium is: 2.3 g/L MS powder+3% sucrose+0.45% phytagel.

4. Molecular Detection of HPPD Doubling Seedlings (T0 Generation):

After the second round of molecular identification, 29 doubling event-positive calli were co-differentiated to obtain 403 seedlings of T0 generation, and the 8F+6R primer pair was used for the third round of molecular identification of the 403 seedlings, among which 56 had positive bands. The positive seedlings were moved into a greenhouse for cultivation. The PCR detection results of some T0 seedlings were shown in FIG. 8.

5. HPPD Inhibitory Herbicide Resistance Test for HPPD Doubled Seedlings (T0 Generation):

The transformation seedlings of T0 generation identified as doubling event positive were transplanted into large plastic buckets in the greenhouse for expanding propagation to obtain seeds of T1 generation. After the seedlings began to tiller, the tillers were taken from vigorously growing strains, and planted in the same pots with the tillers of the wild-type control varieties at the same growth period. After the plant height reached about 20 cm, the herbicide resistance test was conducted. The herbicide used was Bipyrazone (CAS No. 1622908-18-2) produced by our company, and its field dosage was usually 4 grams of active ingredients per mu (4 g a.i./mu). In this experiment, Bipyrazone was applied at a dosage gradient of 2 g a.i./mu, 4 g a.i./mu, 8 g a.i./mu and 32 g a.i./mu with a walk-in spray tower.

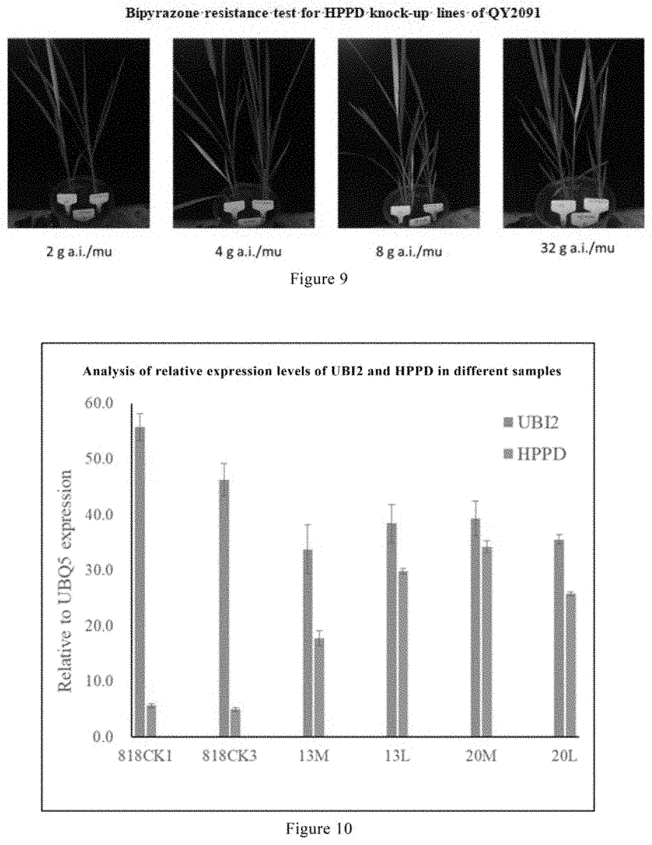

The resistance test results were shown in FIG. 9. After 5-7 days of the application, the wild-type control rice seedlings began to show albino, while the strains of the HPPD doubling events all remained normally green. After 4 weeks of the application, the wild-type rice seedlings were close to death, while the strains of the doubling events all continued to remain green and grew normally. The test results showed that the HPPD gene-doubled strains had a significantly improved tolerance to Bipyrazone.

6. Quantitative Detection of the Relative Expression of the HPPD Gene in the HPPD Doubled Seedlings (T0 Generation):

It was speculated that the improved resistance of the HPPD gene doubled strain to Bipyrazone was due to the fusion of the strong promoter of UBI2 and the HPPD gene CDS that increased the expression of HPPD, so the T0 generation strains QY2091-13 and QY2091-20 were used to take samples from the primary tillers and the secondary tillers used for herbicide resistance test to detect the expression levels of the HPPD and UBI2 genes, respectively, with the wild-type Jinjing 818 as the control. The specific steps were as follows:

1) Extraction of Total RNA (Trizol Method):

0.1-0.3 g of fresh leaves were taken and ground into powder in liquid nitrogen. 1 ml of Trizol reagent was added for every 50-100 mg of tissue for lysis; the Trizol lysate of the above tissue was transferred into a 1.5 ml centrifuge tube, stood at room temperature (15-30° C.) for 5 minutes; chloroform was added in an amount of 0.2 ml per 1 ml of Trizol; the centrifuge tube was capped, shaken vigorously in hand for 15 seconds, stood at room temperature (15-30° C.) for 2-3 minutes, then centrifuged at 12000 g (4° C.) for 15 minutes; the upper aqueous phase was removed and placed in anew centrifuge tube, isopropanol was added in an amount of 0.5 ml per 1 ml of Trizol, the mixture was kept at room temperature (15-30° C.) for 10 minutes, then centrifuged at 12000 g (2-8° C.) for 10 minutes; the supernatant was discarded, and 75% ethanol was added to the pellet in an amount of 1 ml per 1 ml of Trizol for washing. The mixture was vortexed, and centrifuged at 7500 g (2-8° C.) for 5 minutes. The supernatant was discarded; the precipitated RNA was dried naturally at room temperature for 30 minutes; the RNA precipitate was dissolved by 50 μl of RNase-free water, and stored in the refrigerator at −80° C. after electrophoresis analysis and concentration determination.

2) RNA Electrophoresis Analysis:

An agarose gel at a concentration of 1% was prepared, then 1 μl of the RNA was taken and mixed with 1 μl of 2× Loading Buffer. The mixture was loaded on the gel. The voltage was set to 180V and the time for electrophoresis was 12 minutes. After the electrophoresis was completed, the agarose gel was taken out, and the locations and brightness of fragments were observed with a UV gel imaging system.

3) RNA Purity Detection:

The RNA concentration was measured with a microprotein nucleic acid analyzer. RNA with a good purity had an OD260/OD280 value between 1.8-2.1. The value lower than 1.8 indicated serious protein contamination, and higher than 2.1 indicated serious RNA degradation.

4) Real-Time Fluorescence Quantitative PCR

The extracted total RNA was reverse transcribed into cDNA with a special reverse transcription kit. The main procedure comprised: first determining the concentration of the extracted total RNA, and a portion of 1-4 μg of RNA was used for synthesizing cDNA by reverse transcriptase synthesis. The resulting cDNA was stored at −20° C.

\textcircled{1} A solution of the RNA template was prepared on ice as set forth in the following table and subjected to denaturation and annealing reaction in a PCR instrument. This process was conducive to the denaturation of the RNA template and the specific annealing of primers and templates, thereby improving the efficiency of reverse transcription.

TABLE 1
Reverse transcription, denaturation and annealing
reaction system

| Component | Amounts (μl) |
|---|---|
| Oligo dT primer (50 μM) | 1 μl |
| dNTP mixture (10 mM each) | 1 μl |
| RNA Template | l-4 μg |
| RNase free water | Added to 10 μL |

Reaction conditions for denaturation and annealing:

| | |
|---|---|
| 65° C. | 5 min |
| 4° C. | 5 min |

\textcircled{2} The reverse transcription reaction system was prepared as set forth in Table 2 for synthesizing cDNA:

TABLE 2
Reverse transcription reaction system

| Component | Amount (μl) |
|---|---|
| Reaction solution after the above | 10 μl |
| denaturation and annealing | |
| 5× RTase Plus Reaction Buffer | 4 μl |
| RNase Inhibitor | 0.5 μl |
| Evo M-MLV Plus RTase (200 U/μl) | 1 μl |
| RNase free water | Added to 20 μL |

Reaction conditions for cDNA synthesis:

| | |
|---|---|
| 42° C. | 60 min |
| 95° C. | 5 min |

\textcircled{3} The UBQ5 gene of rice was selected as the internal reference gene, and the synthesized cDNA was used as the template to perform fluorescence quantitative PCR. The primers listed in Table 3 were used to prepare the reaction solution according to Table 4.

TABLE 3
Sequence (5′ to 3′) of the primer for
Fluorescence quantitative PCR

| | |
|---|---|
| UBQ5-F | ACCACTTCGACCGCCACTACT |
| UBQ5-R | ACGCCTAAGCCTGCTGGTT |
| RT-OsHPPD-F | CAGATCTTCACCAAGCCAGTAG |
| RT-OsHPPD-R | GAGAAGTTGCCCTTCCCAAA |
| RT-OsUbi2-F | CCTCCGTGGTGGTCAGTAAT |
| RT-OsUbi2-R | GAACAGAGGCTCGGGACG |

TABLE 4
Reaction solution for real-time quantitative PCR
(Real Time PCR)

| Component of mixture | Amount (μl) |
|---|---|
| SYBR Premix ExTaq II | 5 μl |
| Forward primer (10 μM) | 0.2 μl |
| Reverse primer (10 μM) | 0.2 μl |
| cDNA | 1 μl |
| Rox II | 0.2 μl |
| Ultrapure water | 3.4 μl |
| In total | 10 μl |

\textcircled{4} The reaction was performed following the real-time quantitative PCR reaction steps in Table 5. The reaction was conducted for 40 cycles.

TABLE 5
Real-time quantitative PCR reaction steps

| Temperature (° C.) | Time |
|---|---|
| 50° C. | 2 min |
| 95° C. | 10 min |
| 95° C. | 15 s |
| 60° C. | 20 s |
| 95° C. | 15 s |
| 60° C. | 20 s |
| 95° C. | 15 s |

5) Data Processing and Experimental Results

As shown in Table 6, UBQ5 was used as an internal reference, ΔCt was calculated by subtracting the Ct value of UBQ5 from the Ct value of the target gene, and then 2^{-ΔCt} was calculated, which represented the relative expression level of the target gene. The 818CK1 and 818CK3 were two wild-type Jinjing 818 control plants; 13M and 20M represented the primary tiller leaf samples of QY2091-13 and QY2091-20 T0 plants; 13 L and 20 L represented the secondary tiller leaf samples of QY2091-13 and QY2091-20 T0 plants used for herbicide resistance testing.

TABLE 6
Ct values and relative expressionfolds of different genes

| | UBQ5 | Mean | UBI2 | ΔCt | 2^{-ΔCt} | Mean | HPPD | ΔCt | 2^{-ΔCt} | Mean |
|---|---|---|---|---|---|---|---|---|---|---|
| | 23.27 | | 17.56 | −5.88 | 58.95 | | 20.81 | −2.63 | 6.20 | |
| | 23.55 | | 17.71 | −5.73 | 53.09 | | 21.01 | −2.43 | 5.40 | |
| 818CK1 | 23.51 | 23.44 | 17.66 | −5.78 | 55.06 | 55.70 | 20.98 | −2.47 | 5.52 | 5.71 |
| | 23.45 | | 17.88 | −5.50 | 45.20 | | 20.93 | −2.44 | 5.43 | |
| | 23.19 | | 17.94 | −5.44 | 43.41 | | 21.13 | −2.24 | 4.74 | |
| 818CK3 | 23.49 | 23.37 | 17.72 | −5.65 | 50.26 | 46.29 | 21.14 | −2.24 | 4.72 | 4.96 |
| | 24.61 | | 19.56 | −4.92 | 30.32 | | 20.23 | −4.25 | 19.07 | |
| | 24.27 | | 19.52 | −4.96 | 31.05 | | 20.29 | −4.19 | 18.28 | |
| 13M | 24.56 | 24.48 | 19.16 | −5.32 | 39.97 | 33.78 | 20.48 | −4.00 | 15.99 | 17.78 |
| | 23.98 | | 18.76 | −5.20 | 36.70 | | 19.02 | −4.94 | 30.64 | |
| | 23.89 | | 18.52 | −5.43 | 43.19 | | 19.07 | −4.89 | 29.56 | |
| 13L | 24.00 | 23.96 | 18.81 | −5.14 | 35.34 | 38.41 | 19.07 | −4.88 | 29.45 | 29.88 |
| | 24.34 | | 19.01 | −5.40 | 42.30 | | 19.37 | −5.04 | 32.98 | |
| | 24.41 | | 19.07 | −5.34 | 40.64 | | 19.33 | −5.09 | 34.05 | |
| 20M | 24.49 | 24.41 | 19.29 | −5.13 | 35.00 | 39.32 | 19.26 | −5.16 | 35.65 | 34.22 |
| | 24.63 | | 19.46 | −5.11 | 34.52 | | 19.88 | −4.69 | 25.83 | |
| | 24.67 | | 19.38 | −5.19 | 36.48 | | 19.91 | −4.66 | 25.31 | |
| 20L | 24.41 | 24.57 | 19.42 | −5.15 | 35.61 | 35.54 | 19.86 | −4.71 | 26.16 | 25.77 |

The results were shown in FIG. 10. The rice UBQ5 was used as an internal reference gene to calculate the relative expression levels of the OsHPPD and UBI2 genes. The results showed that the HPPD expression level of the HPPD doubled strain was significantly higher than that of the wild type, indicating that the fused UBI2 strong promoter did increase the expression level of HPPD, thereby creating a highly-expressing HPPD gene, with the HPPD gene knocked up. The slight decrease in the expression level of UBI2 could be due to the small-scale mutations resulting from the edition of the promoter region, and we had indeed detected base insertions, deletions or small fragment deletions at the UBI2 target site. Compared with the wild type, the expression levels of UBI2 and HPPD significantly tended to be consistent and met the oretical expectations; among them, the HPPD expression level of the 20M sample was about 6 times higher than that of the wild type CK3 group.

The above results proved that, following the effective chromosome fragment doubling program as tested in protoplasts, calli and transformed seedlings with doubling events could be selected by multiple rounds of molecular identification during the Agrobacterium transformation and tissue culturing, and the UBI2 strong promoter in the new HPPD gene fusion generated in the transformed seedlings did increase the expression level of HPPD gene, rendering the plants to get resistance to HPPD inhibitory herbicide Bipyrazone, up to 8 times the field dose, and thus a herbicide-resistant rice with knock-up endogenous HPPD gene was created. Taking this as an example, using the chromosome fragment doubling technical solution of Example 1 and Example 2, a desired promoter could also be introduced into an endogenous gene which gene expression pattern should be changed to create a new gene, and a new variety of plants with desired gene expression pattern could be created through Agrobacterium-mediated transformation.

### Example 3: Molecular Detection and Herbicide Resistance Test of T1 Generation of Herbicide-Resistant Rice Strain with Knock-Up Expression of the Endogenous HPPD Gene Caused by Chromosome Fragment Doubling

The physical distance between the HPPD gene and the UBI2 gene in the wild-type rice genome was 338 kb, as shown in Scheme 1 in FIG. 1. The length of the chromosome was increased by 338 kb after the chromosome fragment between them was doubled by duplication, and a highly-expressing new HPPD gene was generated with a UBI2 promoter at the joint of the duplicated fragment to drive the expression of the HPPD CDS region. In order to determine whether the new gene could be inherited stably and the effect of the doubling chromosome fragment on the genetic stability, molecular detection and herbicide resistance test was conducted for the T1 generation of the HPPD doubled strains.

First of all, it was observed that the doubling event had no significant effect on the fertility of T0 generation plants, as all positive T0 strains were able to produce normal seeds. Planting test of T1 generation seedlings were further conducted for the QY2091-13 and QY2091-20 strains.

1. Sample Preparation:

For QY2091-13, a total of 36 T1 seedlings were planted, among which 27 grew normally and 9 were albino. 32 were selected for DNA extraction and detection, where No. 1-24 were normal seedlings, and No. 25-32 were albino seedlings.

For QY2091-20, a total of 44 T1 seedlings were planted, among which 33 grew normally and 11 were albino. 40 were selected for DNA extraction and detection, where No. 1-32 were normal seedlings, and No. 33-40 were albino seedlings.

Albino seedlings were observed in the T1 generation plants. It was speculated that, since HPPD was a key enzyme in the chlorophyll synthesis pathway of plants, and the T0 generation plants resulting from the dual-target edition possibly could be chimeras of many genotypes such as doubling, deletion, inversion of chromosome fragments, or small fragment mutation at the edited target site. The albino phenotype could be generated in the plants where the HPPD gene was destroyed, for example, the HPPD CDS region was deleted. Different primer pairs were designed for PCR to determine possible genotypes.

2. PCR Molecular Identification:

1) Sequences of Detection Primers: Sequence 5′-3′

Primer 8F:
TCTGTGTGAAGATTATTGCCACTAGTTC
Primer 6R:
GAGTTCCCCGTGGAGAGGT
Test 141-F:
CCCCTTCCCTCTAAAAATCAGAACAG
Primer 4R:
GGGATGCCCTCCTTATCTTGGATC
Primer 3F:
CCTCCATTACTACTCTCCCCGATTC
Primer 7R:
GTGTGGGGGAGTGGATGACAG
pg-Hyg-R1:
TCGTCCATCACAGTTTGCCA
pg-35S-F:
TGACGTAAGGGATGACGCAC

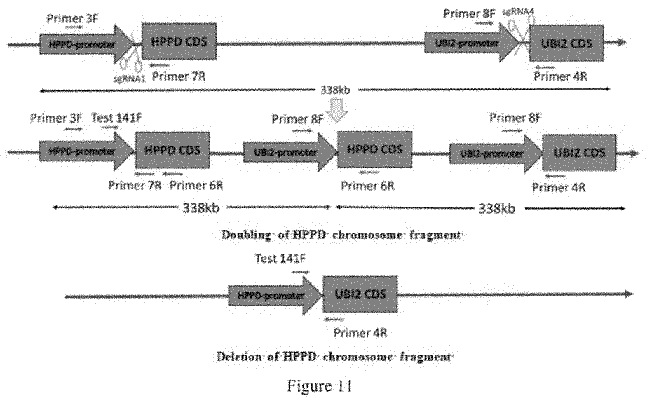

2) The binding sites of the above primers were shown in FIG. 11. Among them, the Primer 8F+Primer 6R were used to detect the fusion fragment of the UBI2 promoter and the HPPD CDS after the chromosome fragment doubling, and the length of the product was 630 bp; the Test 141-F+Primer 4R were used to detect chromosome fragment deletion event, and the length of the product was 222 bp; and the pg-Hyg-R1+pg-35S-F were used to detect the T-DNA fragment of the editing vector, and the length of the product was 660 bp.

3) PCR reaction system, reaction procedure and gel electrophoresis detection were performed according to Example 1.

3. Molecular Detection Results:

The detection results of doubling and deletion events were shown in Table 7. It could be noted that the chromosome fragment doubling events and deletion events were observed in the T1 generation plants, with different rations among different lines. The doubling events in the QY2091-13 (29/32) were higher than that in the QY2091-20 (21/40), possibly due to the different chimeric ratios in the T0 generation plants. The test results indicated that the fusion gene generated by the doubling was heritable.

TABLE 7
Detection results of doubling and deletion events

| QY2091-20 | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Doubling | + | − | − | − | + | + | + | − | − | − | + | − | − | − | − | − | + | + | + | − |
| Deletion | − | − | − | − | − | + | − | − | + | − | − | + | − | − | − | − | − | − | − | − |

| | 21 | 22 | 23 | 24 | 25 | 26 | 27 | 28 | 29 | 30 | 31 | 32 | 33 | 34 | 35 | 36 | 37 | 38 | 39 | 40 |
|---|---|---|---|---|---|---|---|---|---|---|---|---|---|---|---|---|---|---|---|---|
| Doubling | − | + | − | − | + | − | + | + | + | − | + | + | + | − | + | + | + | + | + | − |
| Deletion | − | − | + | + | − | + | − | − | − | + | + | − | + | − | + | − | − | − | − | + |

| QY2091-13 | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Doubling | + | − | + | + | + | + | + | − | + | + | + | + | + | + | + | + | + | + | + | + |
| Deletion | − | + | − | + | + | + | − | + | − | − | + | − | − | − | − | − | − | − | − | + |

| | 21 | 22 | 23 | 24 | 25 | 26 | 27 | 28 | 29 | 30 | 31 | 32 |
|---|---|---|---|---|---|---|---|---|---|---|---|---|
| Doubling | − | + | + | + | + | + | + | + | + | + | + | + |
| Deletion | − | − | + | − | − | + | − | − | + | + | − | + |

The pg-Hyg-R1+pg-35S-F primers were used to detect the T-DNA fragment of the editing vector for the above T1 seedlings. The electrophoresis results of the PCR products of QY2091-20-17 and QY2091-13-7 were negative for the T-DNA fragment, indicating that it was a homozygous doubling. It could be seen that doubling-homozygous non-transgenic strains could be segregated from the T1 generation of the doubling events.

4. Detection of Editing Events by Sequencing:

The doubling fusion fragments were sequenced for the doubling-homozygous positive T1 generation samples 1, 5, 7, 11, 18 and 19 for QY2091-20 and for the doubling-homozygous positive T1 samples 1, 3, 7, 9, 10 and 12 for QY2091-13. The left target site of the HPPD gene and the right target site of the UBI2 were amplified at the same time for sequencing to detect the editing events at the target sites. Among them, the Primer 3F+Primer 7R were used to detect the editing event of the left HPPD target site, where the wild-type control product was 481 bp in length; the Primer 8F+Primer 4R were used to detect the editing event of the right UBI2 target site, where the wild-type control product was 329 bp in length.

1) Genotype of the Doubling Events:

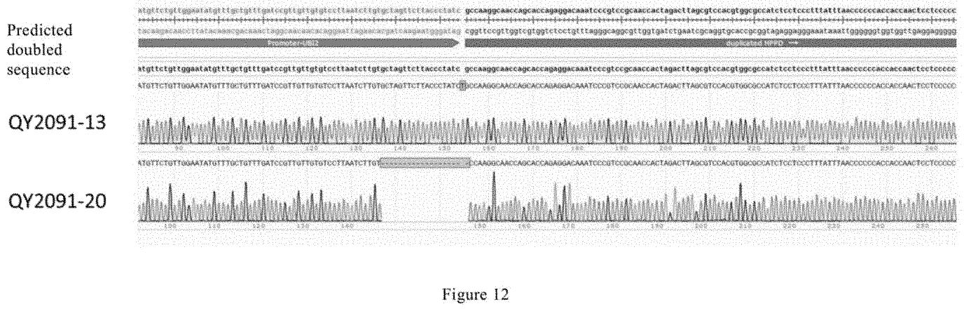

The sequencing result of the HPPD doubling in QY2091-13 was shown in SEQ ID NO: 18, and the sequencing result of the HPPD doubling in QY2091-20 was shown in SEQ ID NO: 19, see FIG. 12. Compared with the predicted linker sequences of the doubling, one T base was inserted at the linker in QY2091-13, 19 bases were deleted from the linker in QY2091-20, and both of the insertion and deletion occurred in the promoter region of UBI2 and had no effect on the coding region of the HPPD protein. From the detection results on the expression levels of the HPPD gene in Example 2, it can be seen that the expression levels of these new HPPD genes where the UBI2 promoters were fused to the HPPD CDS region was significantly increased.

2) Editing Events at the Original HPPD and UBI2 Target Sites on Both Sides:

There were more types of editing events at the target sites on both sides. In two lines, three editing types occurred in the HPPD promoter region, namely insertion of single base, deletion of 17 bases, and deletion of 16 bases; and two editing types occurred in the UBI2 promoter region, namely insertion of 7 bases and deletion of 3 bases. The T1 plants used for testing and sampling were all green seedlings and grew normally, indicating that small-scale mutations in these promoter regions had no significant effect on gene function, and herbicide-resistant rice varieties could be selected from their offspring.

5. Herbicide Resistance Test on Seedlings of T1 Generation:

The herbicide resistance of the T1 generation of the QY2091 HPPD doubled strain was tested at the seedling stage. After the T1 generation seeds were subjected to surface disinfection, they germinated on ½ MS medium containing 1.2 μM Bipyrazone, and cultivated at 28° C., 16 hours light/8 hours dark, in which wild-type Jinjing 818 was used as a control.

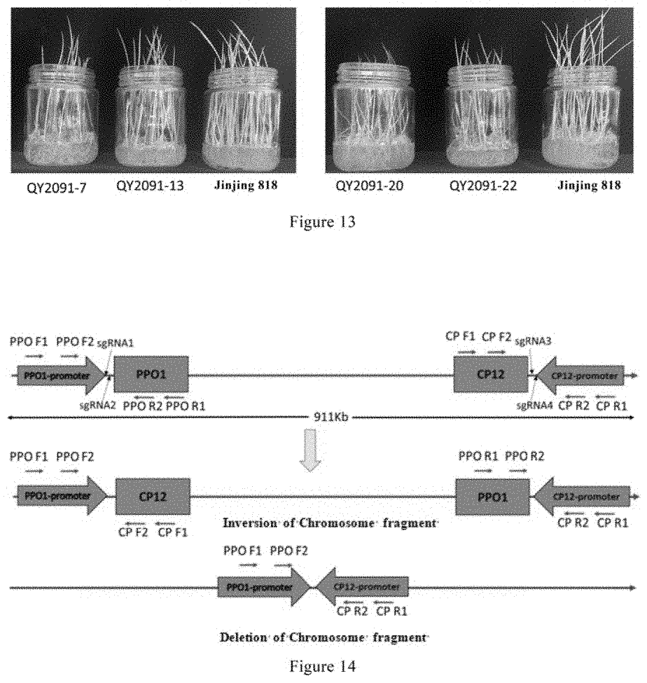

The test results of resistance were shown in FIG. 13. After 10 days of cultivation in light, the wild-type control rice seedlings showed phenotypes of albinism and were almost all albino, while the lines of the HPPD doubling events QY2091-7, 13, 20, 22 showed phenotype segregation of chlorosis and green seedlings. According to the aforementioned molecular detection results, there was genotype segregation in the T1 generation. Albino seedlings appeared in the absence of herbicide treatment, while green seedlings continued to remain green and grew normally after the addition of 1.2 μM Bipyrazone. The test results indicated that the high resistance to Bipyrazone of the HPPD gene-doubled lines could be stably inherited to the T1 generation.

### Example 4: An Editing Method for Knocking Up the Expression of the Endogenous PPO Gene by Inducing Chromosome Fragment Inversion—Rice Protoplast Test

The rice PPO1 (also known as PPOX1) gene (as shown in SEQ ID NO: 7, in which 1-1065 bp was the promoter, the rest was the coding region) was located on chromosome 1, and the calvin cycle protein CP12 gene (as shown in SEQ ID NO: ID NO: 8, in which 1-2088 bp was the promoter, and the rest was the coding region) was located 911 kb downstream of the PPO1 gene with opposite directions. According to the rice gene expression profile data provided by the International Rice Genome Sequencing Project (http://rice.plantbiology.msu.edu/index.shtml), the expression intensity of the CP12 gene in rice leaves was 50 times that of the PPO1 gene, and the CP12 gene promoter was a strong promoter highly expressing in leaves.

As shown in Scheme 1 of FIG. 4, by simultaneously inducing double-strand breaks between the respective promoters and the CDS region of the two genes and screening, the region between the two breaks could be reversed, with the promoter of PPO1 gene replaced with the promoter of CP12 gene, increasing the expression level of the PPO1 gene and achieving the resistance to PPO inhibitory herbicides, thereby herbicide-resistant lines could be selected. In addition, as shown in Scheme 2 of FIG. 4, a new gene of PPO1 driven by the promoter of CP12 gene could also be created by first inversion and then doubling.

1. First, the rice PPO1 and CP12 genomic DNA sequences were input into the CRISPOR online tool (http://crispor.tefor.net/) to search for available editing target sites. After online scoring, the following target sites were selected between the promoters and the CDS regions of the PPO1 and CP12 genes for testing:

| Name of target sgRNA | Sequence (5′ to 3) |
|---|---|
| OsPPO-guide RNA1 | CCATGTCCGTCGCTGACGAG |
| OsPPO-guide RNA2 | CCGCTCGTCAGCGACGGACA |
| OsPPO-guide RNA3 | GCCATGGCTGGCTGTTGATG |
| OsPPO-guide RNA4 | CGGATTTCTGCGTGTGATGT |

The guide RNA1 and guide RNA2 located between the promoter and the CDS region of the PPO1 gene, close to the PPO1 start codon, and the guide RNA3 and guide RNA4 located between the promoter and the CDS region of the CP12 gene, close to the CP12 start codon.

As described in Example 1, primers were designed for the above target sites to construct dual-tar et vectors, with HUE411 as the backbone:

| Primer No. | DNA sequence (5′ to 3′) |
|---|---|
| OsPPO1- | ATATGGTCTCGGGCGCCATGTCCGTCGCTGACGAGGT |
| sgRNA1-F | TTTAGAGCTAGAAATAGCAAG |
| OsPPO1- | ATATGGTCTCGGGCGCCGCTCGTCAGCGACGGACAGT |
| sgRNA2-F | TTTAGAGCTAGAAATAGCAAG |
| OsPPO1- | TATTGGTCTCTAAACCATCAACAGCCAGCCATGGCGC |
| sgRNA3-R | TTCTTGGTGCCGCGCCTC |
| OsPPO1- | TATTGGTCTCTAAACACATCACACGCAGAAATCCGGC |
| sgRNA4-R | TTCTTGGTGCCGCGCCTC |

Specifically, the pCBC-MT1T2 plasmid (https://www.addgene.org/50593/) was used as the template to amplify the sgRNA1+3, sgRNA1+4, sgRNA2+3, sgRNA2+4 dual-target fragments and construct sgRNA expression cassettes, respectively. The pHUE411 vector backbone was digested with BsaI and recovered from gel, and the target fragment was directly used for the ligation reaction after digestion. T4 DNA ligase was used to ligate the vector backbone and the target fragment, the ligation product was transformed into Trans5α competent cells, different monoclones were selected and sequenced. The Sparkjade High Purity Plasmid Mini Extraction Kit was used to extract plasmids with correct sequencing results, thereby obtaining recombinant plasmids, respectively named as pQY002095, pQY002096, pQY002097, pQY002098, as shown below:

pQY002095 pHUE411-PPO-sgRNA1+3 containing OsPPO-guide RNA1, guide RNA3 combination

pQY002096 pHUE411-PPO-sgRNA2+3 containing OsPPO-guide RNA2, guide RNA3 combination

pQY002097 pHUE411-PPO-sgRNA1+4 containing OsPPO-guide RNA1, guide RNA4 combination

pQY002098 pHUE411-PPO-sgRNA2+4 containing OsPPO-guide RNA2, guide RNA4 combination

2. Plasmids of high-purity and high-concentration were prepared for the above-mentioned pQY002095-002098 vectors as described in the step 2 of Example 1.

3. Rice protoplasts were prepared and subjected to PEG-mediated transformation with the above-mentioned vectors as described in step 3 of Example 1.

4. Genomic targeting and detection of new gene with the detection primers shown in the table below for the PCR detection as described in the step 4 of Example 1.

| Primer | Sequence (5′ to 3′) |
|---|---|
| OsPPOinversion- | GCTATGCCGTCGCTCTTTC |
| checkF1(PPO-F1) | TC |
| OsPPOinversion- | CGGACTTATTCCCACCAGA |
| checkF2(PPO-F2) | A |
| OsPPOinversion- | GAGAAGGGGAGCAAGAAGA |
| checkR1(PPO-R1) | CGT |
| OsPPOinversion- | AAGGCTGGAAGCTGTTGGG |
| checkR2(PPO-R2) | |
| OsCPinversion- | CATTCCACCAAACTCCCCT |
| checkF1(CP-F1) | CTG |
| OsCPinversion- | AGGTCTCCTTGAGCTTGTC |
| checkF2(CP-F2) | G |
| OsCPinversion- | GTCATCTGCTCATGTTTTC |
| checkR1(CP-R1) | ACGGTC |
| OsCPinversion- | CTGAGGAGGCGATAAGAAA |
| checkR2(CP-R2) | CGA |

Among them, the combination of PPO-R2 and CP-R2 was used to amplify the CP12 promoter-driven PPO1 CDS new gene fragment that was generated on the right side after chromosome fragment inversion, and the combination of PPO-F2 and CP-F2 was used to amplify the PPO1 promoter-driven CP12 CDS new gene fragment that was generated on the left side after inversion. The possible genotypes resulting from the dual-target editing and the binding sites of the molecular detection primers were shown in FIG. 14.

5. The PCR and sequencing results showed that the expected new gene in which the CP12 promoter drove the expression of PPO1 was created from the transformation of rice protoplasts. The editing event where the rice CP12 gene promoter was fused to the PPO1 gene expression region could be detected in the genomic DNA of the transformed rice protoplasts. This indicated that the scheme to form a new PPO gene through chromosome fragment inversion was feasible, and a new PPO gene driven by a strong promoter could be created, which was defined as a PPO1 inversion event. The sequencing results for the chromosome fragment inversion in protoplasts transformed with the pQY002095 vector were shown in SEQ ID NO: 15; the sequencing results for the chromosome fragment deletion in protoplasts transformed with the pQY002095 vector were shown in SEQ ID NO: 16; and the sequencing results for the chromosome fragment inversion in protoplasts transformed with the pQY002098 vector were shown in SEQ ID NO: 17.

### Example 5: Creation of Herbicide-Resistant Rice with Knock-Up Expression of the Endogenous PPO Gene Caused by Chromosome Fragment Inversion Through Agrobacterium-Mediated Transformation

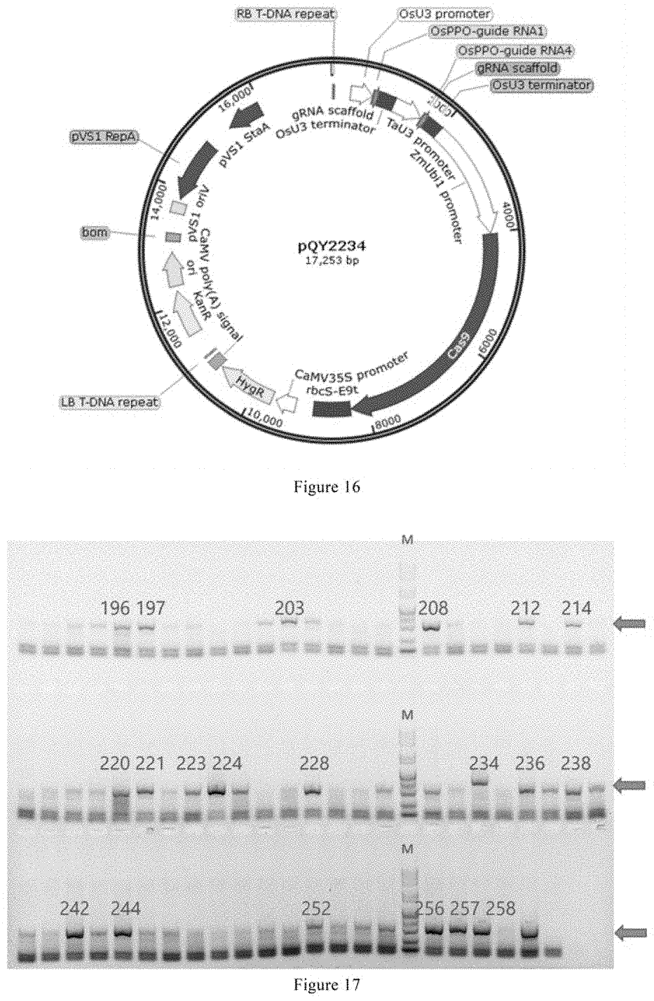

1. Construction of knock-up editing vector: Based on the results of the protoplast testing, the dual-target combination of OsPPO-guide RNA1: 5′CCATGTCCGTCGCTGACGAG3′ and OsPPO-guide RNA4: 5′CGGATTTCTGCGT-GTGATGT3′ with high editing efficiency was selected to construct the Agrobacterium transformation vector pQY2234. pHUE411 was used as the vector backbone and the rice codon optimization was performed. The vector map was shown in FIG. 16.

2. Agrobacterium Transformed Rice Callus and Two Rounds of Molecular Identification:

The pQY2234 plasmid was used to transform rice callus according to the method described in step 2 of Example 2. The recipient varieties were Huaidao No. 5 and Jinjing 818. In the callus selection stage, two rounds of molecular identification were performed on hygromycin-resistant callus, and the calli positive in inversion event were differentiated. During the molecular detection of callus, the amplification of the CP12 promoter-driven PPO1 CDS new gene fragment generated on the right side after chromosome fragment inversion by the combination of PPO-R2 and CP-R2 was deemed as the positive standard for the inversion event, while the CP12 new gene generated on the left side after inversion was considered after differentiation and seedling emergence of the callus. A total of 734 calli from Huaidao No. 5 were tested, in which 24 calli were positive for the inversion event, and 259 calli from Jinjing 818 were tested, in which 29 calli were positive for the inversion event. FIG. 17 showed the PCR detection results of Jinjing 818 calli No. 192-259.

3. A total of 53 inversion event-positive calli were subjected to two rounds of molecular identification and then co-differentiated, and 9 doubling event-positive calli were identified, which were subjected to two rounds of molecular identification and then co-differentiated to produce 1,875 T0 seedlings, in which 768 strains were from Huaidao No. 5 background, and 1107 strains were from Jinjing 818 background. These 1875 seedlings were further subjected to the third round of molecular identification with the PPO-R2 and CP-R2 primer pair, in which 184 lines from Huaidao No. 5 background showed inversion-positive bands, 350 strains from Jinjing 818 background showed inversion-positive bands. The positive seedlings were moved to the greenhouse for cultivation.

4. PPO Inhibitory Herbicide Resistance Test of PPO1 Inversion Seedlings (T0 Generation):

Transformation seedlings of QY2234 T0 generation identified as inversion event-positive were transplanted into large plastic buckets in the greenhouse to grow seeds of T1 generation. There were a large number of positive seedlings, so some T0 seedlings and wild-type control lines with similar growth period and status were selected. When the plant height reached about 20 cm, the herbicide resistance test was directly carried out. The herbicide used was a high-efficiency PPO inhibitory herbicide produced by the company (“Compound A”). In this experiment, the herbicide was applied at the gradients of three levels, namely 0.18, 0.4, and 0.6 g ai/mu, by a walk-in type spray tower.

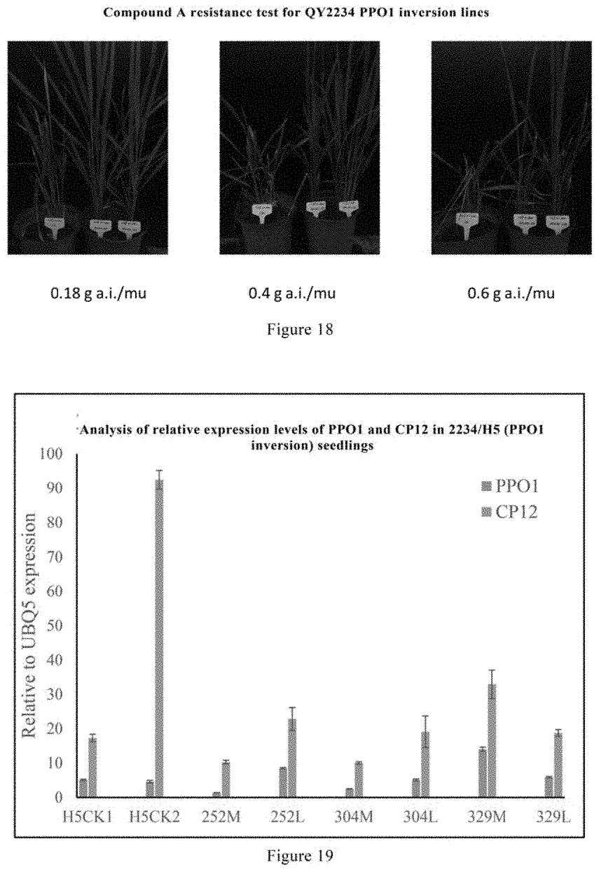

The resistance test results were shown in FIG. 18. 3-5 days after the application, the wild-type control rice seedlings began to wither from tip of leaf, necrotic spots appeared on the leaves, and the plants gradually withered, while most of the lines of the PPO1 inversion event maintained normal growth, the leaves had no obvious phytotoxicity. In addition, some lines showed phytotoxicity, probably due to the polygenotypic mosaicism of editing events and the low expression level of PPO1 in the T0 generation lines. Two weeks after the application, the wild-type rice seedlings died, and most of the inversion event strains continued to remain green and grew normally. The test results showed that the PPO1 inversion lines could significantly improve the tolerance of plants to Compound A.

5. Quantitative Detection of Relative Expression Level of PPO1 Gene in PPO1 Inversion Seedlings (T0 Generation):

It was speculated that the increased resistance of the PPO1 gene inversion lines to Compound A was due to the fusion of the strong CP12 promoter and the CDS of the PPO1 gene which would increase the expression level of PPO1. Therefore, the lines of T0 generation QY2234-252, QY2234-304 and QY2234-329 from Huaidao No. 5 background were selected, their primary tillers and secondary tillers were sampled and subjected to the detection of expression levels of PPO1 and CP12 genes. The wild-type Huaidao No. 5 was used as the control. The specific protocols followed step 6 of Example 2, with the rice UBQ5 gene as the internal reference gene. the fluorescence quantitative primers were as follows: 5′-3′

| | |
|---|---|
| UBQ5-F | ACCACTTCGACCGCCACTACT |
| UBQ5-R | ACGCCTAAGCCTGCTGGTT |
| RT-OsPPO1-F | GCAGCAGATGCTCTGTCAATA |
| RT-OsPPO1-R | CTGGAGCTCTCCGTCAATTAAG |
| RT-OsCP12-F1 | CCGGACATCTCGGACAA |
| RT-OsCP12-R1 | CTCAGCTCCTCCACCTC |

The UBQ5 was used as an internal reference. ΔCt was calculated by subtracting the Ct value of UBQ5 from the Ct value of the target gene. Then 2^{-ΔCt} was calculated, which represented the relative expression level of the target gene. The H5CK1 and H5CK2 were two wild-type control plants of Huaidao No. 5, the 252M, 304M and 329M represented the primary tiller leaf samples of QY2234-252, QY2234-304 and QY2234-329 T0 plants, and the 252 L, 304 L, and 329 L represented their secondary tiller leaf samples. The results were shown in Table 8 below:

TABLE 8
Ct values and relative expression folds of different genes

| | UBQ5 | Mean | PPO1 | ΔCt | 2^{-ΔCt} | Mean | CP12 | ΔCt | 2^{-ΔCt} | Mean |
|---|---|---|---|---|---|---|---|---|---|---|
| | 28.18 | | 25.83 | −2.43 | 5.39 | | 22.28 | −3.98 | 15.77 | |
| | 28.37 | | 25.98 | −2.28 | 4.85 | | 22.06 | −4.20 | 18.44 | |
| H5CK1 | 28.23 | 28.26 | 25.93 | −2.33 | 5.03 | 5.09 | 22.11 | −4.15 | 17.76 | 17.32 |
| | 28.23 | | 25.73 | −2.36 | 5.15 | | 21.63 | −6.47 | 88.58 | |
| | 27.98 | | 26.02 | −2.07 | 4.20 | | 21.53 | −6.57 | 94.87 | |
| H5CK2 | 28.07 | 28.09 | 25.92 | −2.18 | 4.52 | 4.62 | 21.54 | −6.55 | 93.83 | 92.43 |
| | 25.51 | | 25.17 | −0.54 | 1.45 | | 22.26 | −3.45 | 10.95 | |
| | 25.82 | | 25.22 | −0.49 | 1.41 | | 22.36 | −3.36 | 10.23 | |
| 252M | 25.80 | 25.71 | 25.22 | −0.49 | 1.41 | 1.42 | 22.43 | −3.29 | 9.76 | 10.31 |
| | 26.41 | | 23.36 | −3.14 | 8.84 | | 22.30 | −4.21 | 18.49 | |
| | 26.64 | | 23.41 | −3.10 | 8.56 | | 21.95 | −4.56 | 23.55 | |
| 252L | 26.47 | 26.51 | 23.46 | −3.05 | 8.28 | 8.56 | 21.78 | −4.73 | 26.47 | 22.84 |
| | 25.74 | | 24.55 | −1.29 | 2.44 | | 22.51 | −3.32 | 10.02 | |
| | 25.99 | | 24.53 | −1.31 | 2.48 | | 22.45 | −3.39 | 10.47 | |
| 304M | 25.78 | 25.84 | 24.48 | −1.36 | 2.57 | 2.50 | 22.56 | −3.28 | 9.71 | 10.07 |
| | 25.97 | | 23.63 | −2.36 | 5.14 | | 21.60 | −4.39 | 20.97 | |
| | 26.00 | | 23.75 | −2.25 | 4.74 | | 21.43 | −4.56 | 23.55 | |
| 304L | 26.00 | 25.99 | 23.56 | −2.43 | 5.39 | 5.09 | 22.32 | −3.68 | 12.78 | 19.10 |
| | 26.94 | | 23.11 | −3.89 | 14.84 | | 22.23 | −4.76 | 27.16 | |
| | 26.99 | | 23.25 | −3.75 | 13.42 | | 21.85 | −5.15 | 35.39 | |
| 329M | 27.07 | 27.00 | 23.22 | −3.78 | 13.71 | 13.99 | 21.82 | −5.18 | 36.29 | 32.95 |
| | 26.50 | | 23.64 | −2.63 | 6.19 | | 22.00 | −4.27 | 19.30 | |
| | 26.52 | | 23.74 | −2.53 | 5.79 | | 21.97 | −4.30 | 19.71 | |
| 329L | 25.79 | 26.27 | 23.77 | −2.50 | 5.65 | 5.87 | 22.15 | −4.12 | 17.42 | 18.81 |

The relative expression levels of PPO1 and CP12 in different strains were shown in FIG. 19. As the results showed, unlike the doubling event in Example 2, the gene expression levels of these inversion event strains were significantly different. The expression levels of CP12 are very different between the two Huaidao No. 5 CK groups, possibly because of the different growth rates of the seedlings. Compared with the H5CK2 control group, the expression levels of CP12 in the experimental groups all showed a tendency of decrease, while the expression levels of PPO1 for 252 L and 329M increased significantly, and the expression levels of PPO1 for 304 L and 329 L modestly increased, and the expression levels of PPO1 for 252M and 304M decreased. Different from the doubling of chromosome fragments which mainly increased the gene expression level, the inversion of chromosome fragments generated new genes on both sides, so various editing events might occur at the targets on both sides, and the changes in the transcription direction might also affect gene expression level at the same time. That is to say, the T0 generation plants were complex chimeras. There might also be significant differences in gene expression levels between primary and secondary tillers of the same plant. It could be seen from the results of quantitative PCR that the PPO1 inversion events showed a higher likelihood of increasing the PPO1 gene expression level, and thus herbicide-resistant strains with high expression level of PPO1 could be selected out by herbicide resistance selection for the inversion events.

The above results proved that, following the scheme of detecting effective chromosome fragment inversion in protoplasts, calli and transformed seedlings with inversion events could be selected through the multiple rounds of molecular identification during the Agrobacterium transformation and tissue culturing, and the CP12 strong promoter fused with the new PPO1 gene generated in the transformant seedlings could indeed increase the expression level of the PPO1 gene, which could confer the plants with resistance to the PPO inhibitory herbicide Compound A, thereby herbicide-resistant rice with knock-up endogenous PPO gene was created. Taking this as an example, the chromosome fragment inversion protocol of Example 4 and Example 5 also applied to other endogenous genes which gene expression pattern needed to be changed by introducing and fusing with a required promoter, thereby a new gene can be created, and new varieties with a desired gene expression pattern could be created through Agrobacterium-mediated transformation in plants.

### Example 6: Molecular Detection and Herbicide Resistance Test of the T1 Generation Plants of the Herbicide-Resistant Rice Lines with Knock-Up Expression of the Endogenous PPO1 Gene Through Chromosome Fragment Inversion

The physical distance between the wild-type rice genome PPO1 gene and CP12 gene was 911 kb. As shown in FIG. 14, a highly-expressing PPO1 gene with a CP12 promoter-driven PPO1 CDS region was generated on the right side after the inversion of the chromosome fragment between the two genes. A deletion of chromosome fragment could also occur. In order to test whether the new gene could be inherited stably and the influence of the chromosome fragment inversion on genetic stability, molecular detection and herbicide resistance test was carried out on the T1 generation of the PPO1 inversion strain.

First of all, it was observed that the inversion event had no significant effect on the fertility of the T0 generation plants, as all positive T0 strains were able to produce seeds normally. The T1 generations of QY2234/H5-851 strains with the Huaidao No. 5 background were selected for detection.

1. Sample Preparation:

For QY2234/H5-851, a total of 48 T1 seedlings were planted. All the plants grew normally.

2. PCR molecular Identification:

1) Detection Primer Sequence: 5′-3′

PPO-R2:
AAGGCTGGAAGCTGTTGGG
CP-R2:
CTGAGGAGGCGATAAGAAACGA
PPO-F2:
CGGACTTATTTCCCACCAGAA
CP-F2:
AGGTCTCCTTGAGCTTGTCG
pg-Hyg-R1:
TCGTCCATCACAGTTTGCCA
pg-35S-F:
TGACGTAAGGGATGACGCAC

2) The binding sites of the above primers were shown in FIG. 14, wherein the PPO-R2+CP-R2 was used to detect the fusion fragment of the right CP12 promoter and the PPO1 coding region after the inversion of the chromosome fragment, and the length of the product was 507 bp; the PPO-F2+CP-F2 was used to detect the fusion fragment of the left PPO1 promoter and the CP12 coding region after the inversion of the chromosome fragment, and the length of the product was 560 bp; the PPO-F2+PPO-R2 was used to detect the left PPO target site before the inversion, and the length of the product in the wild-type control was 586 bp; the CP-F2+CP-R2 was used to detect the right CP12 target site before the inversion, and the length of the product in the wild-type control was 481 bp. The pg-Hyg-R1+pg-35S-F was used to detect theT-DNA fragment of the editing vector, and the length of the product was 660 bp.

3) PCR reaction system and reaction conditions:

Reaction System (10 μL System):

| | |
|---|---|
| 2*KOD buffer | 5 μL |
| 2 mM dNTPs | 2 μL |
| KOD enzyme | 0.2 μL |
| Primer F | 0.2 μL |
| Primer R | 0.2 μL |
| Water | 2.1 μL |
| Sample | 0.3 μL |

Reaction Conditions:

| 94° C. | 2 minutes | | |
|---|---|---|---|
| 98° C. | 20 seconds | | |
| 60° C. | 20 seconds | {close oversize brace} | 40 cycles |
| 68° C. | 20 seconds | | |

| | |
|---|---|
| 68° C. | 2 minutes |
| 12° C. | 5 minutes |

The PCR products were subjected to electrophoresis on a 1% agarose gel with a voltage of 180V for 10 minutes.

3. Molecular Detection Results:

The detection results were shown in Table 9. A total of 48 plants were detected, of which 12 plants (2/7/11/16/26/36/37/40/41/44/46/47) were homozygous in inversion, 21 plants (1/3/4/5/6/8/9/15/17/20/22/23/24/27/30/31/33/34/39/42/43) were heterozygous in inversion, and 15 plants (10/12/13/14/18/19/21/25/28/29/32/35/38/45/48) were homozygous in non-inversion. The ratio of homozygous inversion: heterozygous inversion:homozygous non-inversion was 1:1.75:1.25, approximately 1:2:1. So the detection results met the Mendel's law of inheritance, indicating that the new PPO1 gene generated by inversion was heritable.

TABLE 9
Results of molecular detection

| QY2234-851 | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Right side of | + | + | + | + | + | + | + | + | + | − | + | − | − | − | + | + | + | − | − | + |
| inversion | | | | | | | | | | | | | | | | | | | | |
| Left side of | + | + | + | + | + | + | + | + | + | − | + | − | − | − | + | + | + | − | − | + |
| inversion | | | | | | | | | | | | | | | | | | | | |
| PPO WT | + | − | + | + | + | + | − | + | + | + | − | + | + | + | + | − | + | + | + | + |
| CP12 WT | + | − | + | + | + | + | − | + | + | + | − | + | + | + | + | − | + | + | + | + |
| QY2234-851 | 21 | 22 | 23 | 24 | 25 | 26 | 27 | 28 | 29 | 30 | 31 | 32 | 33 | 34 | 35 | 36 | 37 | 38 | 39 | 40 |
| Right side of | − | + | + | + | − | + | + | − | − | + | + | − | + | + | − | + | + | − | + | + |
| inversion | | | | | | | | | | | | | | | | | | | | |
| Left side of | − | + | + | + | − | + | + | − | − | + | + | − | + | + | − | + | + | − | + | + |
| inversion | | | | | | | | | | | | | | | | | | | | |
| PPO WT | + | + | + | + | + | − | + | + | + | + | + | + | + | + | + | − | − | + | + | − |
| CP12 WT | + | + | + | + | + | − | + | + | + | + | + | + | + | + | + | − | − | + | + | − |

| QY2234-851 | 41 | 42 | 43 | 44 | 45 | 46 | 47 | 48 |
|---|---|---|---|---|---|---|---|---|
| Right side of | + | + | + | + | − | + | + | − |
| inversion | | | | | | | | |
| Left side of | + | + | + | + | − | + | + | − |
| inversion | | | | | | | | |
| PPO WT | − | + | + | − | + | − | − | + |
| CP12 WT | − | + | + | − | + | − | − | + |

For the above T1 seedlings, the Pg-Hyg-R1+pg-35S-F primers were used to detect the T-DNA fragment of the editing vector. The electrophoresis results of 16 and 41 were negative for T-DNA fragment, indicating homozygous inversion. It could be seen that non-transgenic strains of homozygous inversion could be segregated from the T1 generation of the inversion event.

4. Sequencing Detection of the Editing Events:

The genotype detection of the inversion events focused on the editing events of the new PPO gene on the right side. The mutation events with the complete protein coding frame of the PPO1 gene were retained. The CP12 site editing events on the left side that did not affect the normal growth of plants through the phenotype observation in the greenhouse and field were retained. The genotypes of the editing events detected in the inversion event-positive lines were listed below, in which seamless indicated identical to the predicted fusion fragment sequence after inversion. The genotypes of the successful QY2234 inversion events in Huaidao No. 5 background were as follows:

| No. | Genotype | No. | Genotype |
|---|---|---|---|
| 2234/H5-295 | Right side −1 bp; | 2234/H5-650 | Right side seamless; |
| | left side −32 bp | | left side +1 bp (G) |
| 2234/H5-381 | Right side +18 bp | 2234/H5-263 | Right side seamless; |
| | | | left side seamless |
| 2234/H5-410 | Right side −1 bp; | 2234/H5-555 | Right side −23 bp |
| | left side +1 bp | | |
| 2234/H5-159 | Right side −16 bp | 2234/H5-645 | Right side −5 bp, +20 bp, |
| 2234/H5-232 | Right side −4 bp | | |

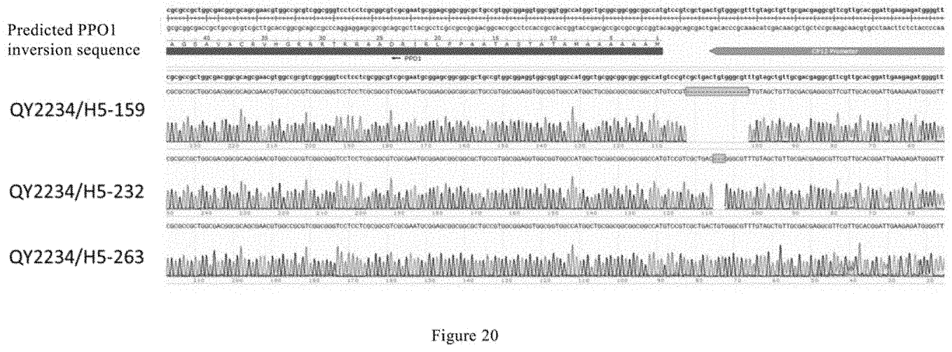

Some of the sequencing peak maps and sequence comparison results were shown in FIG. 20.

The genotypes of the successful QY2234 inversion in the Jinjing818 background were as follows:

| | Right side PPO | | Right side PPO |
|---|---|---|---|
| No. | genotype | No. | genotype |
| 2234/818-5 | Right side seamless | 2234/818-144 | Right side +1 bp |
| 2234/818-42 | Right side −16 bp | 2234/818-151 | Sight side +2 bp, |
| | | | −26 bp, pure peak |
| 2234/818-108 | Right side −15 bp | 2234/818-257 | Sight side +1 bp |

| | |
|---|---|
| 2234/818-134 | Right side +5 bp, |
| | −15 bp |

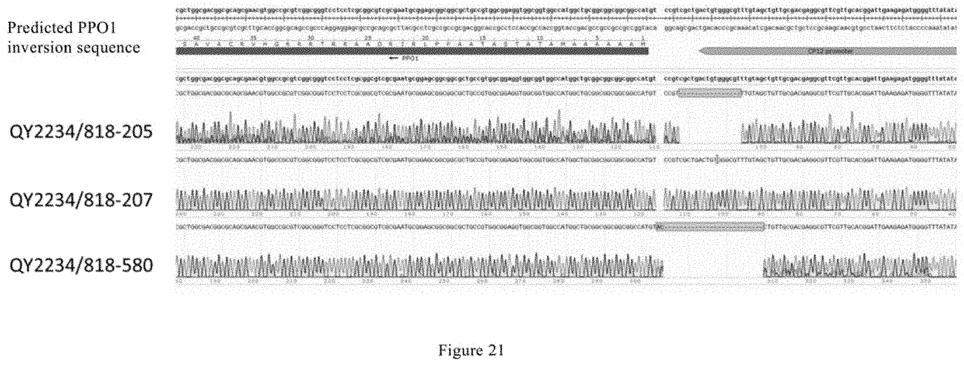

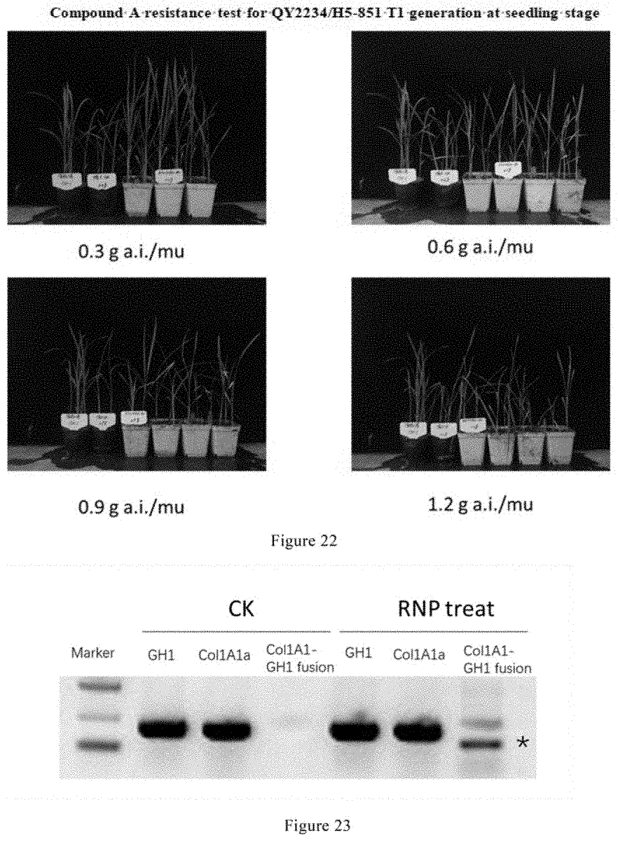

Some of the sequencing peak maps and sequence comparison results were shown in FIG. 21.

The sequencing results of the above different new PPO1 genes with the CP12 promoter fused to the PPO1 coding region were shown in SEQ ID NO: 20, SEQ ID NO: 21, SEQ ID NO: 22, SEQ ID NO: 23, SEQ ID NO: 24, SEQ ID NO: 25, and SEQ ID NO: 26.

5. Herbicide Resistance Test of T1 Generation Seedlings:

The herbicide resistance test was performed on the T1 generation of the QY2234/H5-851 PPO1 inversion lines at seedling stage. The wild-type Huaidao No. 5 was used as a control, and planted simultaneously with the T1 generation seeds of the inversion lines. When the seedlings reached a plant height of 15 cm, Compound A was applied by spraying at four levels of 0.3, 0.6, 0.9 and 1.2 g a.i./mu. The culture conditions were 28° C., with 16 hours of light and 8 hours of darkness.

The resistance test results were shown in FIG. 22. After 5 days of the application, the wild-type control rice seedlings showed obvious phytotoxicity at a dose of 0.3 g a.i./mu. They began to wither from the tip of leaf, and necrotic spots appeared on the leaves; at a dose of 0.6 g a.i./mu, the plants died quickly. However, QY2234/H5-851 T1 seedlings could maintain normal growth at a dose of 0.3 g a.i./mu, and no obvious phytotoxicity could be observed on the leaves; at doses of 0.6 and 0.9 g ai/mu, some T1 seedlings showed dry leaf tips, but most T1 seedlings could keep green and continue to grow, while the control substantially died off. At a dose of 1.2 g a.i./mu, the control plants were all dead, while some of the T1 seedlings could keep green and continue to grow. The test results indicated that the resistance of the PPO1 gene inversion lines to Compound A could be stably inherited to their T1 generation.

### Example 7: An Editing Method for Knocking Up the Expression of the Endogenous EPSPS Gene in Plant

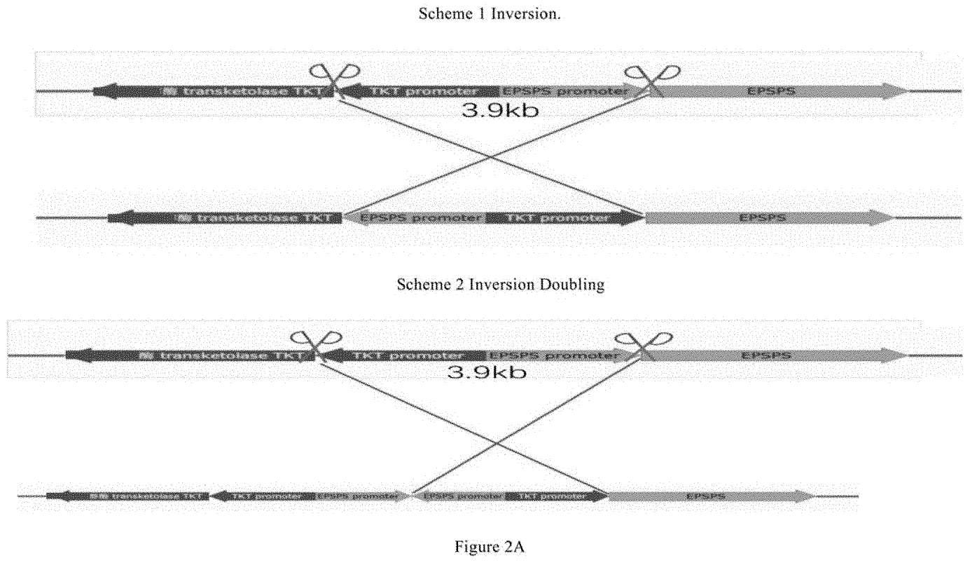

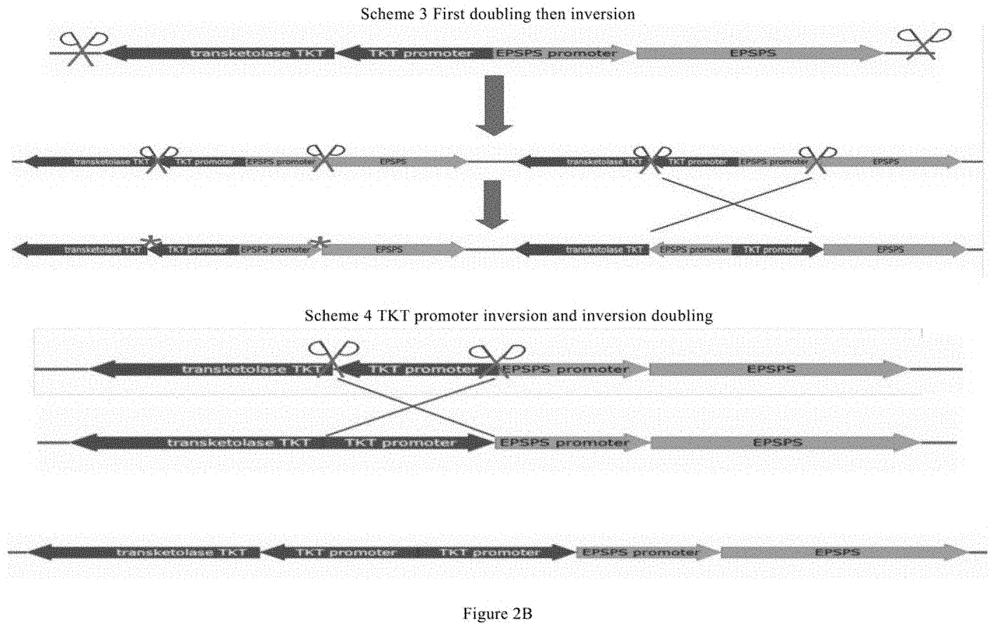

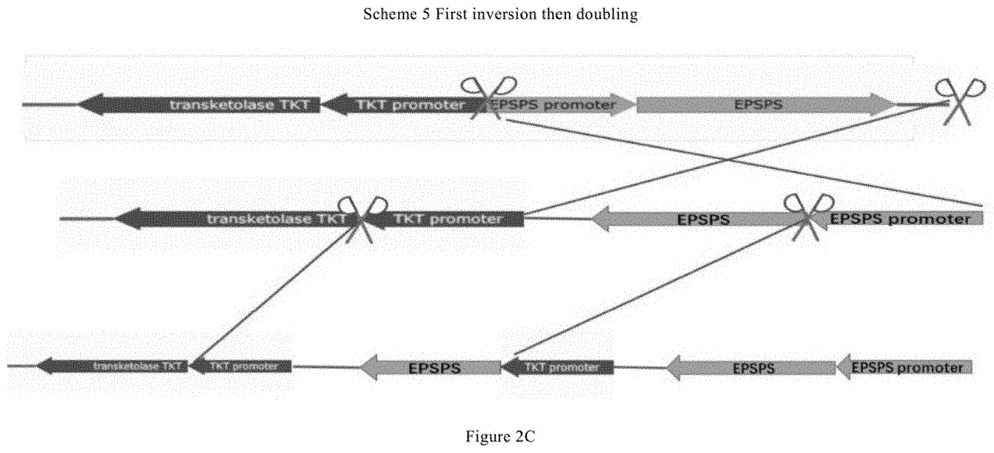

EPSPS was a key enzyme in the pathway of aromatic amino acid synthesis in plants and the target site of the biocidal herbicide glyphosate. The high expression level of EPSPS gene could endow plants with resistance to glyphosate. The EPSPS gene (as shown in SEQ ID NO: 4, in which 1-1897 bp was the promoter, and the rest was the expression region) was located on chromosome 6 in rice. The gene upstream was transketolase (TKT, as shown in SEQ ID NO: 3, in which 1-2091 bp was the promoter, and the rest was the expression region) with an opposite direction. The expression intensity of TKT gene in leaves was 20-50 times that of the EPSPS gene. As shown in FIG. 2, by simultaneously inducing double-strand breaks between the promoter and the CDS region of the two genes respectively, the inversion (Scheme 1) or inversion doubling (Scheme 2) of the region between the two breaks could be obtained after screening. In both cases, the promoter of the EPSPS gene would be replaced with the promoter of the TKT gene, thereby increasing the expression level of the EPSPS gene and obtaining the resistance to glyphosate. In addition, the Schemes 3, 4 and 5 as shown in FIG. 2 could also create new EPSPS genes driven by the TKT gene promoter. The gene structure of EPSPS adjacent to and opposite in direction relative to TKT was conserved in monocotyledonous plants (Table 10). While in dicotyledonous plants, both genes were also adjacent yet in the same direction; therefore, this method was universal in plants.

TABLE 10
Distance between the EPSPS gene and the adjacent TKT
gene in different plants

| | | Distance | |
|---|---|---|---|
| | | from | |
| | | CDS | |
| | | region | |
| | Location | start site | |
| Species | (chromosome) | (kb) | Direction |
| Rice | 6 | 4 | Reverse <TKT-EPSPS> |
| Wheat | 7A | 35 | Reverse <TKT-EPSPS> |
| | 7D | 15 | Reverse <TKT-EPSPS> |
| | 4A? | 50 | Reverse <TKT-EPSPS> |
| Maize | 9 | 22 | Reverse <TKT-EPSPS> |
| Brachypodium | 1 | 5 | Reverse <TKT-EPSPS> |
| distachyon | | | |
| Sorghum | 10 | 15 | Reverse <TKT-EPSPS> |
| Millet | 4 | 5 | Reverse <TKT-EPSPS> |
| Soybean | 3 | 6 | Forward TKT>EPSPS> |
| Tomato | 5 | 6 | Forward TKT>EPSPS> |
| Peanut | 2 | 6 | Forward TKT>EPSPS> |
| | 12 | 5 | Forward TKT>EPSPS> |
| Cotton | 9 | 22 | Forward TKT>EPSPS> |
| Alfalfa | 4 | 8 | Forward TKT>EPSPS> |
| Arabidopsis | 2 | 5 | Forward TKT>EPSPS> |
| Grape | 15 | 17 | Forward TKT>EPSPS> |

To this end, pHUE411 was used as the backbone, and the following as targets:

| Name of target sgRNA | Sequence (5′ to 3′) |
|---|---|
| OsEPSPS-guide RNA1 | CCACACCACTCCTCTCGCCA |
| OsEPSPS-guide RNA2 | CCATGGCGAGAGGAGTGGTG |
| OsEPSPS-guide RNA3 | ATGGTCGCCGCCATTGCCGG |
| OsEPSPS-guide RNA4 | GACCTCCACGCCGCCGGCAA |
| OsEPSPS-guide RNA5 | TAGTCATGTGACCATCCCTG |
| OsEPSPS-guide RNA6 | TTGACTCTTTGGTTCATGCT |

Several different dual-target vectors had been constructed:
pQY002061 pHUE411-EPSPS-sgRNA1+3
pQY002062 pHUE411-EPSPS-sgRNA2+3
pQY002063 pHUE411-EPSPS-sgRNA1+4
pQY002064 pHUE411-EPSPS-sgRNA2+4
pQY002093 pHUE411-EPSPS-sgRNA2+5
pQY002094 pHUE411-EPSPS-sgRNA2+6

(2) With the relevant detection primers shown in the following table, the fragments containing the target sites on both sides or the predicated fragments generated by the fusion of the TKT promoter and the EPSPS coding region were amplified, and the length of the products is between 300-1000 bp.

| Primer | Sequence (5′ to 3′) |
|---|---|
| EPSPSinversion checkF1 | ATCCAAGTTACCCCCTCTGC |
| EPSPSinversion checkR1 | CACAAACACAGCCACCTCAC |
| EPSPSinversion check-nestF2 | ATGTCCACGTCCACACCATA |
| EPSPSinversion check-nestR2 | AATGGAATTCACGCAAGAGG |
| EPSPSinversion checkF3 | GTAGGGGTTCTTGGGGTTGT |
| EPSPSinversion checkR3 | CGCATGCTAACTTGAGACGA |
| EPSPSinversion check-nestF4 | GGATCGTGTTCACCGACTTC |
| EPSPSinversion check-nestR4 | CCGGTACAACGCACGAGTAT |
| EPSPSinversion checkF5 | GGCGTCATTCCATGGTTGAT |
| | TGT |
| EPSPSinversion checknestF6 | GATAGACCCAGATGGGCATA |
| | GAATC |
| EPSPSinversion checkR5 | TGCATGCATTGATGGTTGGT |
| | GC |
| EPSPSinversion checknestR6 | CCGGCCCTTAGAATAAAGGT |
| | AGTAG |

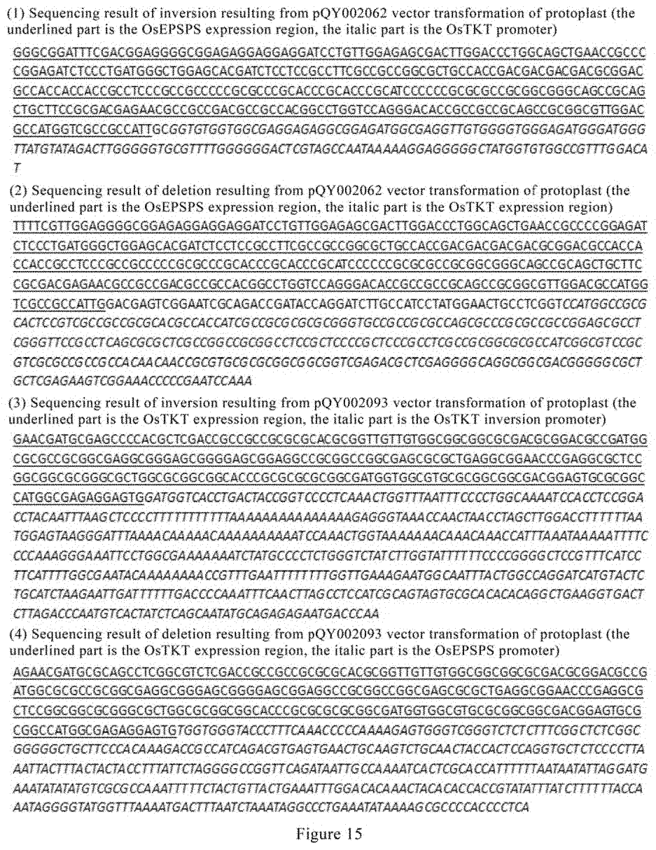

After protoplast transformation, the detection results showed that the expected inversion events were obtained. As shown in FIG. 15, the sequencing result of the inversion detection of pQY002062 vector transformed protoplast was shown in SEQ ID NO: 11; the sequencing result of the deletion detection of pQY002062 vector transformed protoplast was shown in SEQ ID No: 12; the sequencing result of the inversion detection of the pQY002093 vector transformed protoplast was shown in SEQ ID NO: 13; and the sequencing result of the deletion detection of pQY002093 vector transformed protoplast was shown in SEQ ID NO: 14.

These vectors were transferred into Agrobacterium for transforming calli of rice. Plants containing the new EPSPS gene were obtained. The herbicide bioassay results showed that the plants had obvious resistance to glyphosate herbicide.

### Example 8: An Editing Method for Knocking Up the Expression of the Endogenous PPO Gene in Arabidopsis

Protoporphyrinogen oxidase (PPO) was one of the main targets of herbicides. By highly expressing plant endogenous PPO, the resistance to PPO inhibitory herbicides could be significantly increased. The Arabidopsis PPO gene (as shown in SEQ ID NO: 1, in which 1-2058 bp was the promoter, and the rest was the expression region) located on chromosome 4, and the ubiquitin10 gene (as shown in SEQ ID NO: 2, in which 1-2078 bp was the promoter, and the rest was the expression region) located 1.9M downstream with the same direction as the PPO gene.

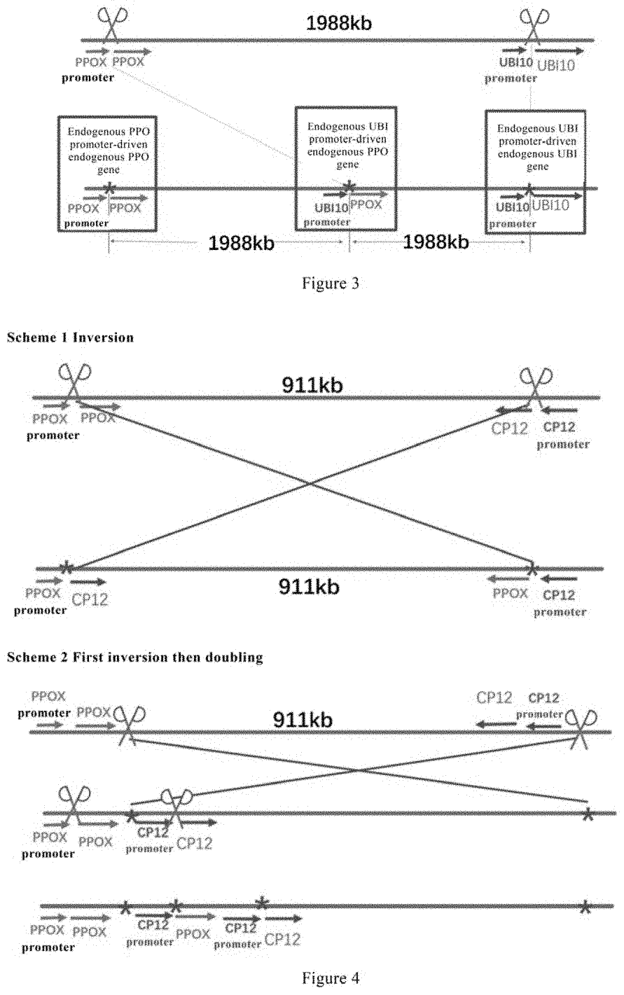

As shown in the Scheme as shown in FIG. 3, simultaneously generating double-strand breaks at the sites between the promoter and the CDS region of the PPO and the ubiquitin10 genes respectively. Doubling events of the region between the two breaks could be obtained by screening, namely a new gene generated by fusing the ubiquitin10 promoter and the PPO coding region. In addition, following Scheme 2 as shown in FIG. 1, a new gene in which the ubiquitin10 promoter and the PPO coding region were fused together could also be created.

To this end, pHEE401E was used as the backbone (https://www.addgene.org/71287/), and the following locations were used as target sites:

| Name of target sgRNA | Sequence (5′ to 3′) |
|---|---|
| AtPPO-guide RNA1 | CAAACCAAAGAAAAAGTATA |
| AtPPO-guide RNA2 | GGTAATCTTCTTCAGAAGAA |
| AtPPO-guide RNA3 | ATCATCTTAATTCTCGATTA |
| AtPPO-guide RNA4 | TTGTGATTTCTATCTAGATC |

The dual-target vectors were constructed following the method described by “Wang Z P, Xing H L, Dong L, Zhang H Y, Han C Y, Wang X C, Chen Q J. Egg cell-specific promoter-controlled CRISPR/Cas9 efficiently generates homozygous mutants for multiple target genes in Arabidopsis in a single generation. Genome Biol. 2015 Jul. 21; 16:144.”:

| | |
|---|---|
| pQY002076 | pHEE401E-AtPPO-sgRNA1 + 3 |
| pQY002077 | pHEE401E-AtPPO-sgRNA1 + 4 |
| pQY002078 | pHEE401E-AtPPO-sgRNA2 + 3 |
| pQY002079 | pHEE401E-AtPPO-sgRNA2 + 4 |

Arabidopsis was transformed according to the method as follows:

(1) Agrobacterium Transformation

Agrobacterium GV3101 competent cells were transformed with the recombinant plasmids to obtain recombinant Agrobacterium.

(2) Preparation of Agrobacterium Infection Solution

1) Activated Agrobacterium was inoculated in 30 ml of YEP liquid medium (containing 25 mg/L Rif and 50 mg/L Kan), cultured at 28° C. under shaking at 200 rpm overnight until the OD600 value was about 1.0-1.5.

2) The bacteria were collected by centrifugation at 6000 rpm for 10 minutes, and the supernatant was discarded.

3) The bacteria were resuspended in the infection solution (no need to adjust the pH) to reach OD600=0.8 for later use.

(3) Transformation of Arabidopsis

1) Before the plant transformation, the plants shouldgrow well with luxuriant inflorescence and no stress response. The first transformation could be carried out as long as the plant height reached 20 cm. When the soil was dry, watering was carried out as appropriate. On the day before the transformation, the grown siliques were cut with scissors.

2) The inflorescence of the plant to be transformed was immersed in the above solution for 30 seconds to 1 minute with gentle stirring. The infiltrated plant should have a layer of liquid film thereon.

3) After transformation, the plant was cultured in the dark for 24 hours, and then removed to a normal light environment for growth.

4) After one week, the second transformation was carried out in the same way.

(4) Seed Harvest

Seeds were harvested when they were mature. The harvested seeds were dried in an oven at 37° C. for about one week.

(5) Selection of Transgenic Plants

The seeds were treated with disinfectant for 5 minutes, washed with ddH₂O for 5 times, and then evenly spread on MS selection medium (containing 30 μg/ml Hyg, 100 μg/ml Cef). Then the medium was placed in a light incubator (at a temperature of 22° C., 16 hours of light and 8 hours of darkness, light intensity 100-150 μmol/m²/s, and a humidity of 75%) for cultivation. The positive seedlings were selected and transplanted to the soil after one week.

(6) Detection of T1 Mutant Plants

(6.1) Genomic DNA Extraction

1) About 200 mg of Arabidopsis leaves was cut and placed into a 2 ml centrifuge tube. Steel balls were added, and the leaves were ground with a high-throughput tissue disruptor.

2) After thorough grinding, 400 μL of SDS extraction buffer was added and mixed upside down. The mixture was incubated in 65° C. water bath for 15 minutes, and mixed upside down every 5 minutes during the period.

3) The mixture was centrifuged at 13000 rpm for 5 minutes.

4) 300 μL of supernatant was removed and transferred to a new 1.5 ml centrifuge tube, an equal volume of isopropanol pre-cooled at −20° C. was added into the centrifuge tube, and then the centrifuge tube was kept at −20° C. for 1 hour or overnight.

5) The mixture was centrifuged at 13000 rpm for 10 minutes, and the supernatant was discarded.

6) 500 μL of 70% ethanol was added to the centrifuge tube to wash the precipitate, the washing solution was discarded after centrifugation (carefully not discarding the precipitate). After the precipitate was dried at room temperature, 30 μL of ddH₂O was added to dissolve the DNA, and then stored at −20° C.

(6.2) PCR Amplification

With the extracted genome of the T1 plant as template, the target fragment was amplified with the detection primers. 5 μL of the amplification product was taken and detected by 1% agarose gel electrophoresis, and then imaged by a gel imager. The remaining product was directly sequenced by a sequencing company.

The sequencing results showed that the AtPPO1 gene doubling was successfully achieved in Arabidopsis, and the herbicide resistance test showed that the doubling plant had resistance to PPO herbicides.

### Example 9: Creation of GH1 Gene with New Expression Characteristics in Zebrafish

The growth hormone (GH) genes in fishes controlled their growth and development speed. At present, highly expressing the GH gene in Atlantic salmons through the transgenic technology could significantly increase their growth rates. The technique was of great economical value, but only approved for marketing after decades. The GH1 gene was the growth hormone gene in zebrafish. In the present invention, suitable promoters in zebrafish (suitable in terms of continuous expression, strength, and tissue specificity) were fused together with the CDS region of GH1 gene in vivo through deletion, inversion, doubling, inversion doubling, chromosome transfer, etc., to create a fast-growing fish variety.

The experiment procedure was as follows:

1. Breeding of Zebra Fish:

1) Preparation of paramecia: The mother liquor of paramecia was purchased online (https://item.taobao.com/item.htm?spm=a230r. 1.14.49.79f774c6C6elpL&id=573612042855 &ns=1&abbucket=18 #detail). A 2 L beaker was washed, sterilized and filled with 200 mL of paramecia mother liquor; two yeast pieces and two sterilized grains of wheat were added thereto; sterile water was added until the volume reaches 2 L; then the opening was covered and sealed with sterilized kraft paper; stationary culture was performed at 25-28° C. for 3-5 d; the mixture was used to feed the juvenile zebra fish when the usable concentration was reached. Each time the paramecia solution was taken, a dense filter screen was used to remove impurities.

2) Incubation of brine shrimp: Brine shrimp, also known as fairy shrimp and artemia, was a marine plankton. Brine shrimp eggs were purchased and stored at 4° C. For the incubation, the mixture was prepared at a ratio of 1 L deionized water: 32 g NaCl:3.5 g brine shrimp eggs; oxygenation was performed at 28.5° C. for 25-30 h; the incubated brine shrimps were collected. The incubated brine shrimps were kept in a small amount of 3.2% NaCl solution, where they could be kept for 2-3 d at 4° C.

3) The standardized large-scale breeding of zebra fish was realized with an independent zebrafish farming system manufactured by Shanghai Haisheng. The tap water treated with a water purifier was kept in a dosing barrel, where an appropriate amount of NaCl and NaHCO₃ was added to maintain a specific conductivity of 500 μs/cm and a pH of 7.0. The water circulation system ensured all breeding tanks maintain a constant water level and flow state. A waste treatment system automatically filters the fish feces and remaining fish food; the fish culture water was reused after being sterilized by UV exposure and heated (28.5° C.); the fresh water was automatically replenished after the wastewater was discharged. The lighting was controlled with an automatic timer in fish house to maintain the “14 h-light+10 h-dark cycles”; an air conditioning system kept the indoor temperature at 28° C.; an exhaust fan removed indoor moisture at regular intervals to avoid excessively high humidity. Zebra fish embryos were subjected to stationary culture in a biochemical incubator at 28.5° C., and could be fed with paramecia 5 days after fertilization. Feeding was performed 3-4 times a day. Fresh brine shrimp started to be supplemented gradually after about 13 days. When the bodies of all juveniles became red, it means the zebra fish can completely eat brine shrimp. The juvenile zebra fish was then transferred to the breeding tank. A moderate amount of fresh brine shrimp was fed 3 times a day.

AB varieties of zebra fish were transferred into an incubation box on a 2-female: 2-male basis on the afternoon of the day before reproduction of zebra fish; they were separated by a baffle. The baffle was removed the next morning; the zebra fish generally began to lay eggs in about 10 minutes; embryos were collected within 30 minutes after egg laying and rinsed with E3 culture medium (mass ratio 29.3% NaCl, 3.7% CaCl₂), 4% MgSO₄, 1.3% KCl, pH7.2) to remove dead eggs.

4) Preparation of injection dish: 1.5% agarose was prepared; 30-40 ml of agarose melt was poured into each plastic culture dish. The surface of agarose was gently covered with the mold to avoid bubbles. The mold was removed after the gel got completely solidified to attain a “V-shaped” groove; a small amount of E3 culture medium was added into the prepared culture dish; it was sealed and kept at 4° C.

2. Preparation of RNP Sample:

For zebrafish GH1 gene initiation codon upstream 100 bp DNA sequence designed sgRNA-GH1 target: 5′aagaacgagtttgtctatct3′, for zebrafish col1a1a gene termination codon designed sgRNA-col1a1a target: 5′atgtagactctttgaggcga3′, and for zebrafish ddx5 gene initiation codon upstream designed sgRNA-ddx5 target: 5′gcaccatcactgcgcgtaca3′. Genscript was entrusted to synthesize the EasyEdit sgRNA. The synthetic sgRNA and the purified Cas9 were mixed at a ratio of 1:3; 10×Cas9 buffer solution (200 mM HEPES, 100 mM MgCl₂, 5 mM DTT, 1.5 M KCl) was added and RNase-free ultra-pure water was used to dilute it to 1× so that the Cas9 protein concentration was 600 ng/uL, and the sgRNA concentration was 200 ng/uL; after 10 minutes of incubation at 25° C., a small amount of phenol red was added to dye the injection sample for convenient observation during injection; the volume of phenol red was normally less than 10% of the total volume. In the experiment, sgRNA-GH1 combined with sgRNA-col1a1a and sgRNA-ddx5, respectively and the RNP complex was prepared at equal ratio, and the mixture was injected into fish eggs.

3. Microinjection:

Under a stereomicroscope, the tip of injection needle was fractured slightly in a beveled manner using medical pointed toothless forceps for convenient injection. 4 μL of sample containing phenol red was taken by a micro loading tip; the pipette tip inserts into the needle from the end of needle to the tip reaching the point of injection needle; the tip was gently pushed to inject the sample into the needle while the tip was gently pulled out so that the front end of injection needle was filled with the red RNP sample; the injection needle with sample was then inserted into the holder for fixation. The quantitative capillary was 33 mm in length and 1 μL in total volume. The injection pressure was adjusted to increase the length of the liquid column in the capillary by 1 mm after 15 injections; then, each sample injection volume was 1 nL. The injection dish was taken out of the refrigerator in advance, and set aside until the room temperature was reached. The collected one-cell stage fertilized eggs were arranged in the groove of dish; a small amount of E3 culture medium was added so that the liquid level was just over the fertilized eggs. Under a stereomicroscope, the tip of the injection needle was gently penetrated into the membrane of the fertilized egg and reaches the yolk close to the animal pole; RNP sample was injected by stepping on the pedal. Due to the small amount of phenol red in the RNP sample, the light red sample liquid can be clearly observed during the injection. The injected embryos were placed in a disposable plate containing E3 culture medium and cultured in a constant temperature incubator at 28.5° C. The culture medium needs to be replaced every 24 hours to ensure the ion concentration and oxygen content.

4. DNA Extraction:

After each set of injections, the tail fin of survived zebrafish at about 2-3 months old were treated with cell lysate buffer (10 mmol/L Tris, 10 mmol/L EDTA, 200 mmol/L NaCl, 0.5% SDS, 200 μg/mL Proteinase K, pH 8.2). Each tube was filled with 200 μL of lysate and held overnight at 50° C.; they were violently shaken 2-3 times during this period. The tube was centrifuged at 1200 r/min for 5 min at room temperature, and then 200 μL of supernatant was taken. Equal volume of phenol: chloroform: isoamyl alcohol (25:24:1) was added and violently shaken. The tube was centrifuged at 12000 r/min at room temperature for 10 min; the supernatant taken was mixed with equal volume of chloroform, and then the tube was violently shaken. The tube was centrifuged at 12000 r/min at room temperature for 10 min; the supernatant was mixed with 1/20-volume 3 mol/L NaCl and 2.5-time volume pre-cooled anhydrous ethanol; the mixture was blended well and should not be made upside down; it's kept on ice for 30 min. The tube was centrifuged at 12000 r/min at 4° C. for 10 min, and the supernatant was abandoned swiftly. 1 mL 70% alcohol was added for rinsing. The tube was centrifuged at 12000 r/min at 4° C. for 10 min. The supernatant was abandoned swiftly, and vacuum drying was performed. Finally, 30 μL of deionized water was added in the end to dissolve the DNA. The solution was kept at −20° C. for future use. After the 0.8% agarose electrophoresis detection, the PCR test was performed with the corresponding primer, and the positive strip was subjected to sequencing verification. Wherein, gh1-R: tgctacaaataaagtgcactacaca and col1a1a-F:gggtctggattggagtcaca were double treated between the amplified col1a1a gene and gh1; gh1-R:tgctacaaataaagtgcactacaca and ddx5-F:acgcgttacgtacgtcagaa, as well as GH1-F:aaatgaccggaatcacaaca and ddx5-R:acgaccatccttaccctctg were inversely treated between the amplified ddx5 gene and gh1.

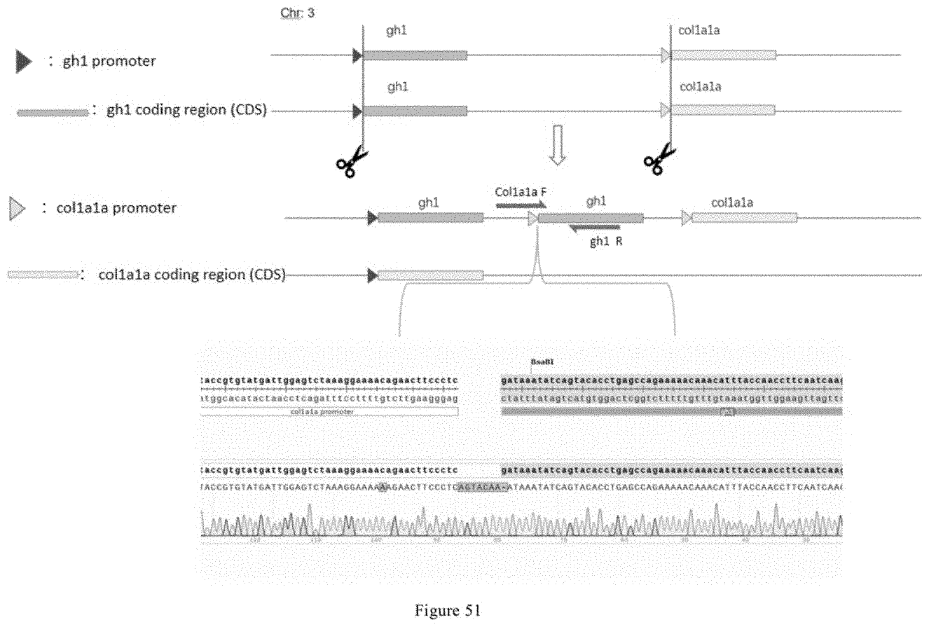

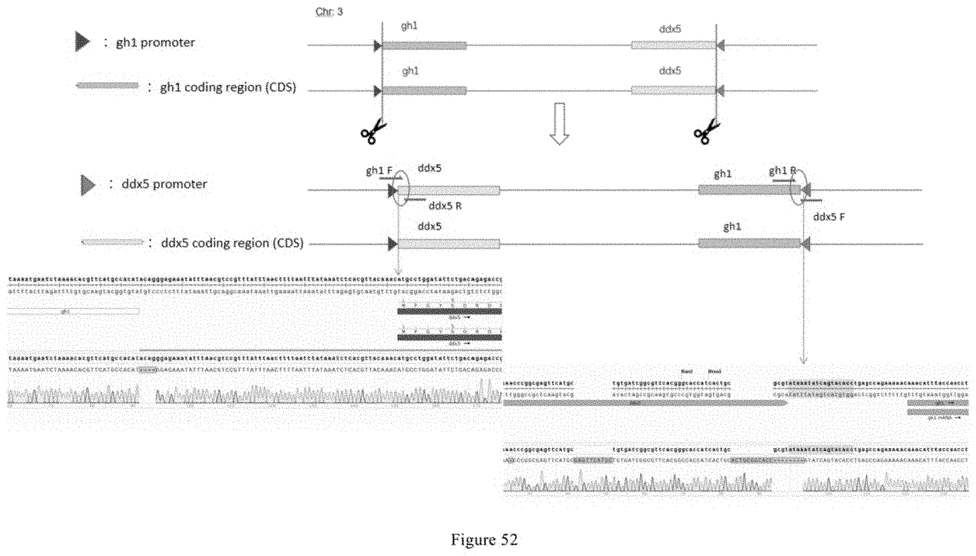

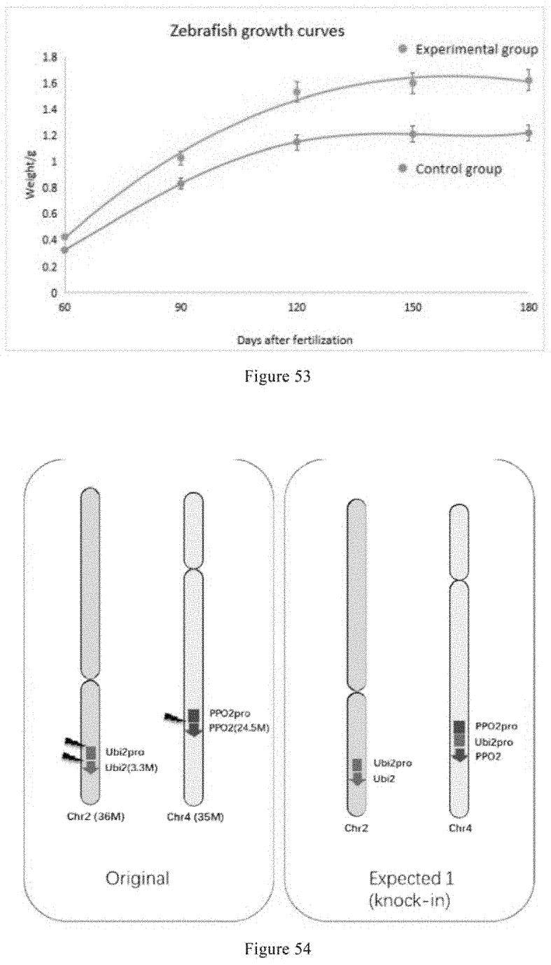

The experimental results were shown as follows: as shown in FIG. 23, the characteristic fragments of chromosome duplication were detected in the zebrafish embryo samples of the RNP injection group; as shown in FIG. 51, sequencing results showed that the expected duplication event occurred in the chromosome fragments between GH1 and COL1A1A gene targets in zebrafish embryos; as shown in FIG. 52, sequencing results showed that the coding area & promotor of ddx5 gene and the coding area & the promotor of gh1 gene were exchanged due to the inversion of chromosome fragments; as shown in FIG. 53, the growth of zebrafish with upregulated expression was obviously accelerated.

### Example 10: Field Herbicide Resistance Test on T1 Generation of Herbicide-Resistant Rice Lines QY2234

T1 generation of inversion lines QY2234/818-5 and QY2234/818-42 PPO1 were subjected to field herbicide resistance test with the wild-type Jinjing 818 rice variety as an herbicide-susceptible control. They were planted in sync with the inversion line T1 generation seeds in a paddy field of Red Flag Team, Nanbin Farm, Sanya, Hainan Province; the test was performed between Nov. 30, 2020 and Apr. 15, 2021. Seedlings were cultivated after 2 days of soaking rice seeds, and transplantation was performed after 3 weeks of seedling; 3 sets of replications were arranged; an herbicide called as compound A was applied in 3 weeks after transplantation, and the concentration was set to 0.3, 0.6 and 0.9 ga.i./mu (1 mu= 1/15 ha); the status of rice seedlings was investigated 10 days post application (DPA).

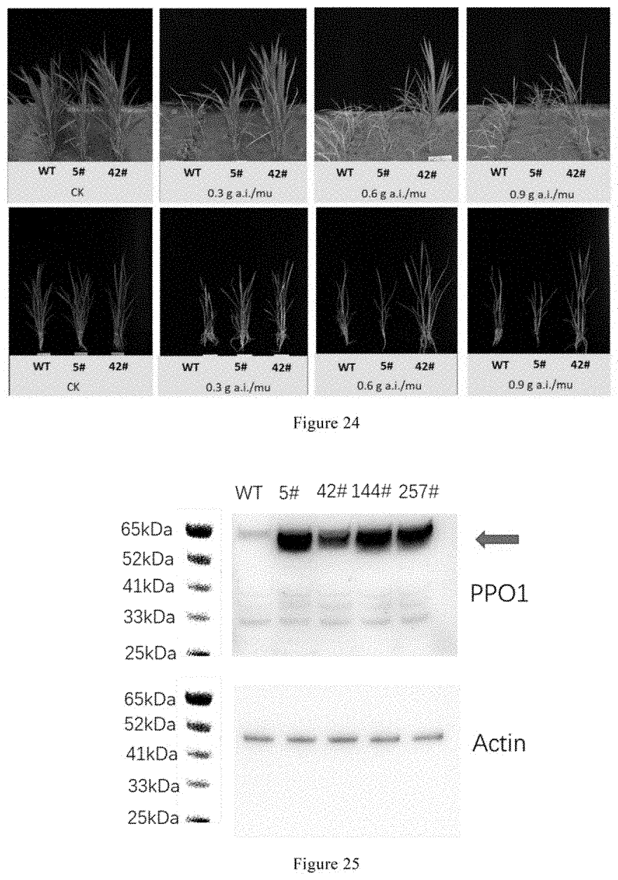

The result of the field herbicide resistance test was shown in FIG. 24. In 10 DPA at a dose of 0.3 ga.i./mu, all wild-type Jinjing 818 rice plants died; QY2234/818-5 and QY2234/818-42 were growing normally; at the dose of 0.6 ga.i./mu, QY2234/818-42 was growing normally, while most individual plants of QY2234/818-5 died, but there were resistant individual plants of QY2234/818-5; at the dose of 0.9 ga.i./mu, most individual plants of QY2234/818-42 and QY2234/818-5 died, but a few resistant plants were green and continued to grow. The test result indicated that the PPO1 gene inverted line exhibited herbicide resistance under field conditions under high light intensity in Hainan; stable resistant lines can be selected from the populations, which provides basic materials for herbicide-resistant rice breeding.

### Example 11: Western Blot Test on T1-Generation PPO1 Protein Expression Level of the QY2234 Line Rice

The T1-generation seedling leaves of the four PPO1 inversion rice lines, i.e., QY2234/818-5, QY2234/818-42, QY2234/818-144 and QY2234/818-257, were selected to determine the PPO1 protein expression level. With the wild-type Jinjing 818 rice variety as a control, they were planted in the greenhouse in sync with the inversion line T1 generation seeds; when the seedlings grew to a height of 15 cm, leaf samples were taken with reference to Example 6 for molecular identification; the inversion-positive seedlings were selected for the Western Blot Test on protein expression.

A Western Blot test was performed as per the Molecular Cloning: A Laboratory Manual (Sambrook, J., Fritsch, E. F. and Maniatis, T, Molecular Cloning: A Laboratory Manual; Cold Spring Harbor Laboratory Press: Cold Spring Harbor, 1989). The PPO1 protein antibody was rice PPO1 polyclonal antibody prepared by Qingdao Jinmotang Biotechnology Co., Ltd. (Qingdao, China); a plant endogenous reference Actin protein antibody was purchased from Sangon Biotech (Shanghai, China) Co., Ltd. (Art. No. D195301); the secondary antibody was HRP-labeled goat anti-rabbit IgG (Sangon, Art. No. D110058); the test was performed according to the operating instructions using the Western Blot Kit (Boster, Art. No. AR0040).

To be more specific, 2 g of single-plant rice sample was taken and ground with liquid nitrogen into powder; an appropriate amount of protein extraction buffer (material: Protein extraction buffer=1:1.5); incubated on ice for 30 min; centrifuged at 4° C. with 27100 g for 15 min; the supernatant was mixed with 5× loading buffer (delivered with the kits); the mixture was boiled for 15 min and subjected to electrophoresis at 110 V for 30 min.

The protein extraction buffer was formulated as follows:

| component | concentration |
|---|---|
| Tris-HCl (PH8.0) | 100 mM |
| glycerin | 10% |
| EDTA | 1 mM |
| AsA (ascorbic acid) | 2 mM |
| PVPP | 0.5% |
| PVP-40 | 0.5% |
| DTT (Add at operation time) | 20 mM |
| PMSF (Add at operation time) | 1 mM |

After the electrophoresis was finished, the gel was removed, and the gel block in an appropriate size was taken depending on the size of target protein, and then the filter paper and PVDF film of approximately the same volume was taken; the gel block was cleaned with clear water and then soaked with transfer solution; the filter paper and PVDF film were also soaked and wetted with the transfer solution; the wet filter paper, PVDF film, SDS-PAGE gel block and filter paper were stacked from bottom to top to expel as many bubbles as possible; they could be flattened with a test tube, while the displacement between layers should be prevented during the flattening; the film was transferred under at 25 V and 1.3 A for 10-30 min; after the transfer, the PVDF film was cleaned with PBST buffer. Upon completion of the cleaning, they were transferred to the blocking buffer solution and blocked at room temperature for 1 h. After the confining, the PVDF film was cleaned with PBST buffer solution for 3 times to remove the blocking liquid, then the primary antibody was incubated at a dilution ratio of approx. 1:1000-1:3000; the incubation time of the primary antibody was 2 h at room temperature, or 12 h at 4° C. Upon completion of the primary antibody incubation, the PVDF film was cleaned with the PBST buffer solution for 3 times with each cycle lasting for 10 min. The secondary antibody was incubated at a dilution ratio of approx. 1:10000-1:20000 for 1 h at room temperature. Upon completion of the secondary antibody incubation, the PVDF film was cleaned with the PBST buffer solution for 3 times with each cycle lasting for 10 min. ECL luminescence: ECL solutions A and B were mixed well in equal volume (prepared when needed), and the liquid mixture was dropped onto the PVDF film evenly; the film was placed in the fluorescence imager for imaging.

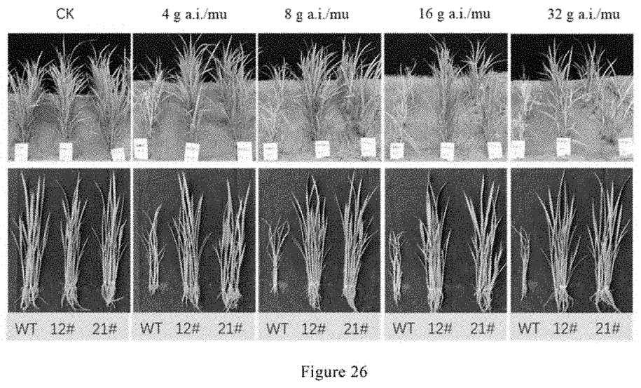

The Western Blot test result was shown in FIG. 25. According to the result, the internal-control Actin protein expression levels of the PPO1 inversion-positive lines were the same as the wild-type Jinjing 818, while the expression levels of PPO1 protein were significantly up-regulated. The 4 selected QY2234 lines had different genotypes at the inverted junction region between the CP12 promoter and the PPO1 protein coding region; QY2234/818-5 was identical to the predicted post-inverted fusion fragment sequence; compared with the predicted sequence, QY2234/818-42 lacks 16 bases in the CP12 promoter region; 1 base was inserted in the CP12 promoter region of QY2234/818-144 and QY2234/818-257. The test result showed that all the new genotypes could express PPO1 protein at high levels, and manifested that the method for creating new genes provided by the present invention could produce a variety of functional genotypes in the genome to enrich the gene pool.

### Example 12: Field Herbicide Resistance Test on T1 Generation of HPPD-Duplicated Rice Lines QY2091

Through germination test, the T1-generation of HPPD-gene duplicated lines QY2091-12 and QY2091-21 without albino seedling separation were selected for the field herbicide resistance test with the wild-type Jinjing 818 rice variety as a control. They were planted in sync with the T1 generation seeds of QY2091 lines in a paddy field of Red Flag Team, Nanbin Farm, Sanya, Hainan Province; the test was performed between Nov. 1, 2020 and Apr. 10, 2021. Seedlings were cultivated after 2 days of soaking rice seeds, and QY2091 seeds were soaked with 1/30000 herbicide compound Bipyrazone aqueous solution; albino seedlings were removed after emergence; transplantation was performed after 3 weeks of seedling, and 3 sets of repetitions were arranged; herbicide compound Bipyrazone was applied in 3 weeks after the transplantation at a concentration of 4, 8, 16, and 32 ga.i./mu; the seedling status was investigated 21 days after application.

The result of field herbicide resistance test was shown in FIG. 26. In 21 days after application, all wild-type Jinjing 818 rice plants died of albinism at 4 ga.i./mu and 8 ga.i./mu of herbicide compound Bipyrazone, while the QY2091-12 and QY2091-21 were normally growing; at 16 g a.i./mu, the QY2091-12 line was growing normally, while the QY2091-21 line began to exhibit resistance separation: Most individual plants showed resistance, and a few individual plants developed yellowing of new leaves; at 32 g a.i./mu, QY2091-12 and QY2091-21 began to exhibit resistance separation, and a few individual plants died of albinism, while significant yellowing of new leaves was observed in some individual plants; approx. ½ of the individual plants were growing normally and exhibit extremely high resistance. The recommended dosage for field application of herbicide compound Bipyrazone was 4 g a.i./mu. The test result indicated that the edited lines with highly expressive HPPD new genes created through chromosome segment duplication exhibited herbicide resistance under field conditions under high light intensity in Hainan, and could withstand an herbicide dose that was 8 times the recommended field dose. The screening of stable resistant lines will provide basic materials for herbicide-resistant rice breeding.

### Example 13: New Gene Creation Activity of NLS-Free Cas9 and Separately Expressed crRNA and tracrRNA in Rice Protoplast

Targets were chosen from upstream and downstream of the PPO1 gene to test whether chromosome fragment duplication events could be produced; furthermore, tests were performed on whether the nuclear localization signal with Cas9 removed could produce duplication event, and on whether replacing sgRNA (single guide RNA) with separated expression of crRNA and tracrRNA could induce cell targeted site editing to produce chromosome fragment duplication events.

Dual-target editing vector pQY2648 was constructed by the method described in Example 1 for the selected target sequence design primers, i.e., OsPPO1-esgRNA3:5′ taggtctccaaacATG GCGTTTTCTGTCCGCGTgcttcttggtgccgcg3′ and OsPPO1-esgRNA2:5′ TaggtctccggcgCAGTT GGATTAGGGAATATGGTTTAAGAGCTATGCTGGAAACAGC3′. The NLS signal peptides at both ends of SpCas9 wereremoved on the basis of pQY2648 to construct the NLS-free rice PPO1 dual-target editing vector pQY2650; the sgRNA expression cassette was modified based on pQY2650 and pQY2648; the fused Scaffold sequence was removed, and the crRNA and tracrRNA sequences were separately expressed. To be more specific, the OsU3 promoter drove the expression of OsPPO1-sgRNA2:5′CAGTTGGATTAGGGAATATGGTTTAAGGCTATGCT3′ crRNA sequence; the TaU3 promoter drove the expression of the OsPPO1-sgRNA3:5′ ACGCGGACAGAAAACGCCATGTTTAAGGCTATGC3′ sequence; the OsU3 promoter drove the expression of the expression cassette of tracrRNA sequence 5′AGCATAGCAAGTTTAAATAAGGCTAGTC CGTTATCAACTTGAAAAAGTGGCACCGAGTCGGTGCTTT3′; the NLS-free crRNA rice PPO1 dual-target editing vector pQY2651 and the crRNA rice PPO1 dual-target editing vector pQY2653 containing NLS were constructed; the primers used during the process were as follows:

2650F-BstBI:
5′gtacaaaaaagcaggcttcgaaATGgacaagaagtactcgatcggc3′
2650R-SacI:
5′tgaacgatcggggaaattcgagctcCTAgtcgcccccgagctgag3′
OsU3-HindIII-For2651F:
5′GCAGGTCTCaagcttaaggaatctttaaacatacgaacag3′
CrRNA1-BsaI-R1:
5′GCAGGTCTCCAGGTAAAAAAAAAAAGCATAGCCTTAAACCATATTCCC
TAATCCAACTG3′
TaU3-BsaI-F2:
5′GCAGGTCTCCACCcatgaatccaaaccacacggag3′
CrRNA2-BsaI-R2:
5′GCAGGTCTCGCTAGAAAAAAAAAAGCATAGCCTTAAACATGGCGTTTT
CTGTCCGCGT3′
TraCrRNA-OsU3-BsaIF3:
5′GCAGGTCTCGCTAGaaggaatctttaaacatacgaac3′
TraCrRNA-KpnI-R3:
5′GgtaccAAAAAAAAAAAAGCACCGACTCGGTGCCACTTTTTCAAGTTG
ATAACGGACTAGCCTTATTTAAACTTGCTATGCTCGCCacggatcatctg
cacaac3′,

For the above-mentioned 4 vectors, Example 1 was consulted to prepare the high-purity and high-concentration plasmids for PEG-mediated transformation of rice protoplast; the protoplast DNA was extracted for detection of edit duplication event; PCR primers to amplify the designed duplicated DNA at the junction regions were designed based the targeted cut sites on both sides, and then the PCR amplified fragments were sequenced; the primer sequences were as follows:

OsPPO1Dup-testF1:
CCACTGCTGCCACTTCCAC
OsPPOlDup-testF2:
GGCGACTTAGCATAGCCAG
OsPPO1Dup-testR1:
GCTATTGCGGTGCGTATCC
OsPPOlDup-testR2:
TCCAAGCTAGGGGTGAGAGA

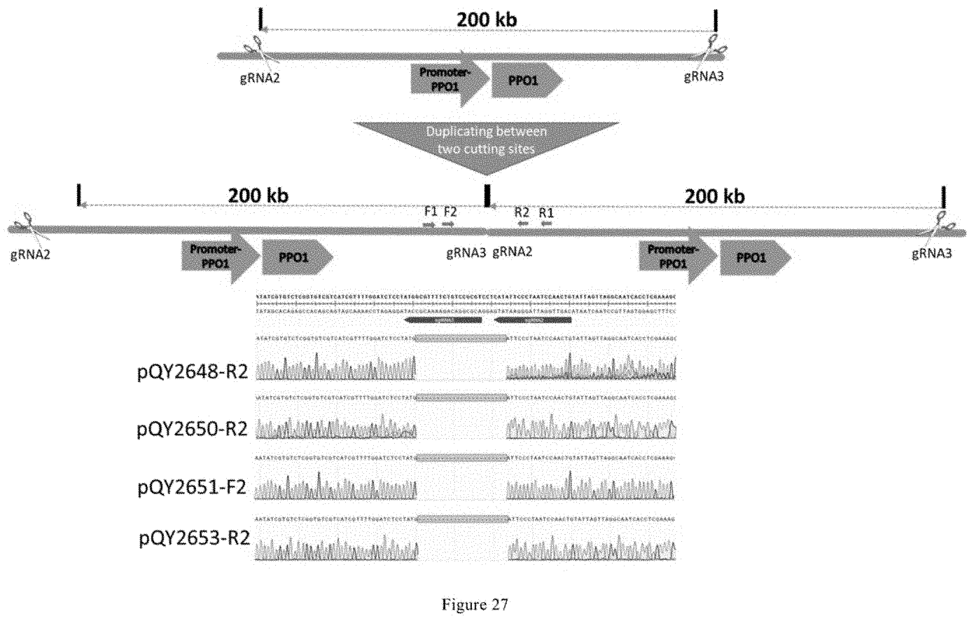

The test result was shown in FIG. 27; chromosome fragment duplication events were detected through the sequencing of PCR products using primers OsPPO1Dup-testF2 or OsPPO1Dup-testR2 and DNA extracted from pQY2648, pQY2650, pQY2651 and pQY2653 transformed rice protoplast samples; small fragments of DNA were missing between two DNA break sites at the expected fragment junction regions. The protoplast test result of pQY2650 demonstrated that the Cas9 without NLS could cut the target effectively to produce and detected the doubling event of chromosome fragments between targeted cuts when chromosomes were edited with the dual-target editing vector. The result of pQY2653 protoplast test demonstrates that the assembled gRNA could effectively guide the target editing in the event of separately expressed crRNA and tracrRNA to produce and detect doubling event of chromosome fragments between targeted cutting sites. The pQY2651 protoplast test result demonstrated that NLS-free Cas9 could work with separately expressed crRNA and tracrRNA and the spontaneously assembled gRNA to effectively guide target editing to produce and detect doubling event of chromosome fragments between cuts, which indicated that the creation of new genes through doubling/duplication, inversion, or translocation of chromosome fragments using the method of the present invention was independent of the nuclear localization signal of Cas9 protein or the fused single guide RNA (sgRNA) system.

### Example 14: Different Chromosome Fragment Translocation and Restructure to Create a New HPPD Gene in Rice

As mentioned in example 1 and example 4, rice HPPD gene is located on chromosome 2, CP12 gene is located on chromosome 1 but in opposite direction. Through CRISPR/Cas9-mediated chromosome cutting and naturally occurred inversion of CP12 and PPO1 gene protein coding regions-containing fragment and followed by the chromosomal fragments fusion, a new gene was generated in which CP12 promoter drives PPO1 expression, and as expected PPO1 expression was significantly enhanced, and conferred rice plant herbicide resistance. Taking advantage of the high expression characteristics of the CP12 promoter, a dual-target editing vector was designed and constructed, which cut the two regions upstream two start codons ATGs. After Agrobacterium-mediated transformation and followed by selection and plant-regeneration, a new HPPD gene in which CP12 promoter drives HPPD protein expression was identified through PCR and amplicon sequencing.

According to the analysis of rice gene expression profile data (http://rice.plantbiology.msu.edu/index.shtml) provided by the international rice genome sequencing project (International Rice Genome Sequencing Project), CP12 gene expression intensity is dozens to hundred times that of HPPD gene in rice leaf blade, CP12 gene promoter is strong in leaf blades and seedlings.

With reference to example 1 and example 2, the related genomic DNA sequences of rice HPPD and CP12 genes were input into CRISPOR online tool (http://crispor.tefor.net/) to find and assess available edit targets. After online scoring, the following targets (5′-3′) were selected between the promoters and protein coding regions of HPPD and CP12 genes for testing:

| | |
|---|---|
| HPPD-guide RNA1 | gtgctggttgccttggctgc |
| HPPD-guide RNA2 | cacaaattcaccagcagcca |
| CP12-guide RNA1 | gccatggctggctgttgatg |
| CP12-guide RNA2 | cggatttctgcgtgtgatgt |

HPPD-guide RNA1 and HPPD-guide RNA2 are located between HPPD gene promoter and protein coding region and close to HPPD protein start codon ATG, while CP12-guide RNA1 and CP12-guide RNA2 are located between CP12 gene promoter and protein coding region and close to CP12 protein start codon ATG.

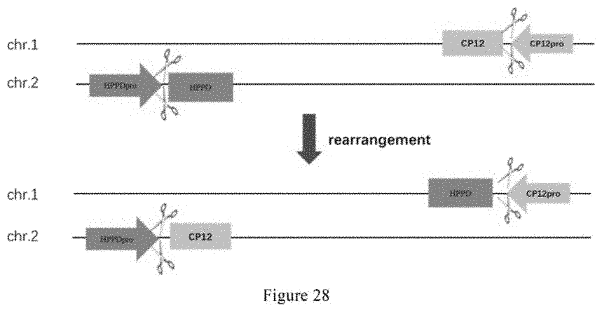

For the above-mentioned targets the following primers were designed and synthesized, the double-target editors pQY2257, pQY2258, pQY2259, pQY2260 were constructed with expectation of the editing events in which CP12 promoter driving HPPD protein coding region could be identified after transformation and selection with hyg, as shown in FIG. 28.

| | DNA sequence (5′ to 3′) |
|---|---|
| Primer ID | target sequences are underlined |
| HPPD-sgRNA1-F | taggtctccggcggtgctggttgccttggctgcgt |
| | tttagagctagaaatagcaagttaaaataaggc |
| HPPD-sgRNA2-F | taggtctccggcgcacaaattcaccagcagccagt |
| | tttagagctagaaatagcaagttaaaataaggc |
| CP12-sgRNA1-R | taggtctccaaaccatcaacagccagccatggcgc |
| | ttcttggtgccgcg |
| CP12-sgRNA2-R | taggtctccaaacacatcacacgcagaaatccggc |
| | ttcttggtgccgeg |

Wherein, guide RNA combinations in each editing vector:

pQY2257 contains the combination of HPPD-guide RNA1 and CP12-guide RNA1,

pQY2258 contains the combination of HPPD-guide RNA1 and CP12-guide RNA2 combination,

pQY2259 contains the combination of HPPD-guide RNA2 and CP12-guide RNA1 combination,

pQY2260 contains the combination of HPPD-guide RNA2 and CP12-guide RNA2 combination.

With reference to the example 1 for rice protoplast transformation method, the above pQY2257-2260 vectors with high purity and concentration of the plasmid DNA were prepared, and then the high-quality rice protoplast was prepared as well, PEG mediated transformation of the rice protoplast was carried out, and finally the genome editing and the designed new gene was expected to be detected where CP12 promoter drives HPPD gene expression.

The following detecting primers, OsCP12pro-detection-F and OsHPPDutr-detection-R, were used to amplify the predicted fragment generated by the fusion of CP12 promoter and HPPD coding region, and the length of PCR amplicon was expected to be 305 bp. Similarly, OsHPPDpro-detection-F and OsCP12cds-detection-R were used to detect the fragment produced by the fusion of HPPD promoter and CP12 coding region, and the length of PCR products was expected to be 445 bp.

| Primer ID | Sequence (5′ to 3′) |
|---|---|
| OsCP12pro-detection-F | ctgaggaggcgataagaaacga |
| OsHPPDutr-detection-R | gtgtgggggagtggatgac |
| OsHPPDpro-detection-F | caagagctttactccaagttacc |
| OsCP12cds-detection-R | acccgccctcggagttgg |

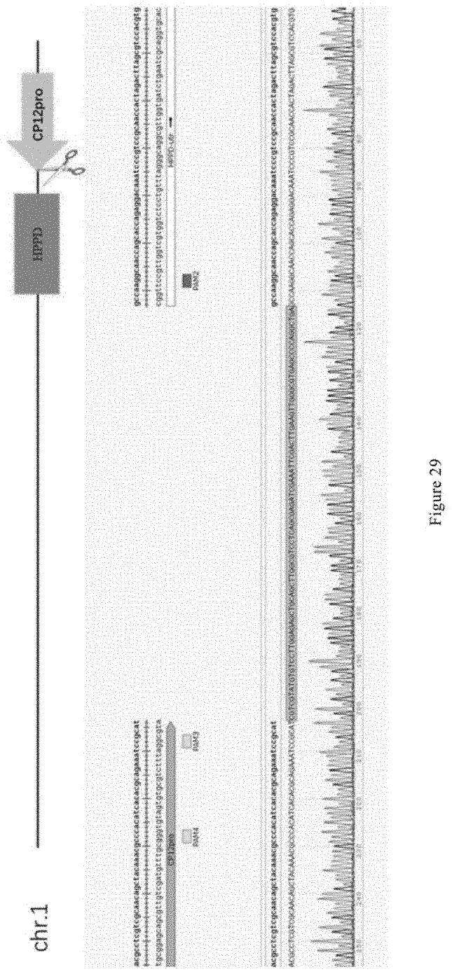

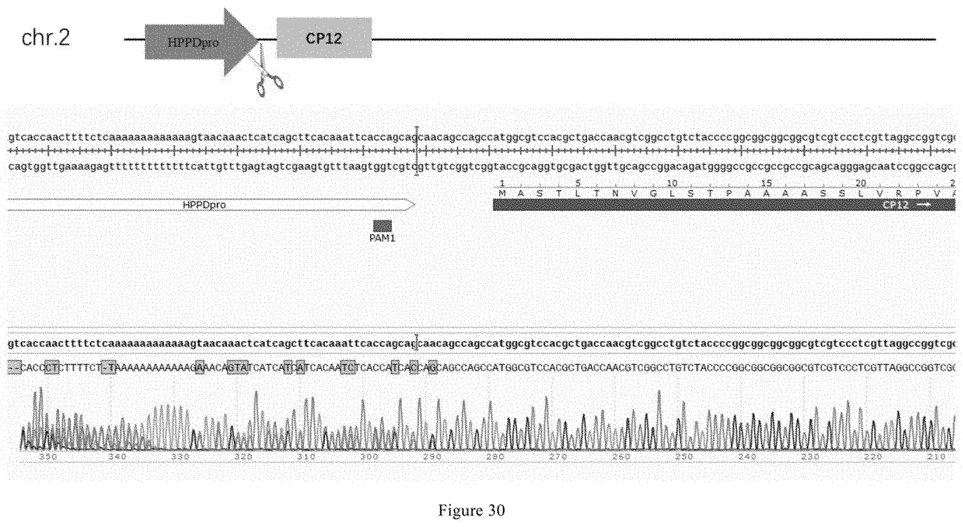

The identification results showed that in pQY2257-transformed protoplast samples were detected to have the CP12 promoter fused with the HPPD coding region, as shown in FIG. 29. While in the pQY2259-transformed protoplast samples were detected to have the HPPD promoter fused with the CP12 coding region, as shown in FIG. 30.

The above results show that, using the method described in this invention, can generate recombination between two chromosome fragments derived from two different chromosomes, which is expected to create the new genes as designed.

In this particular example, HPPD gene expression increases driven by the strong promoter of CP12 gene, meanwhile CP12 gene expression decreases driven by the weak promoter of HPPD gene. Therefore, the expression level of the new genes generated through this invention can be regulated as needed by choice of a strong or weak promoter.

### Example 15: Creation of a New High-Expression HPPD Gene Caused by Chromosome Fragment Duplication Mediated by LbCpf1 Dual-Target Editing-Rice Protoplast Test

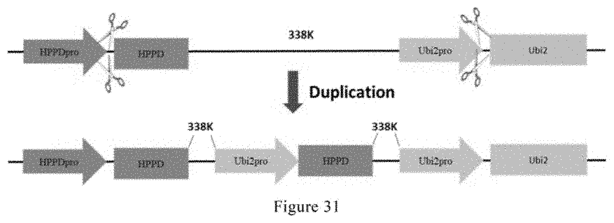

LbCpf1 belongs to the Cas12a type of nucleases, recognizes a TTTV PAM site, and thus is suitable to edit a high AT-content DNA sequence; while Cas9 recognizes a NGGPAM site and is suitable to edit a high GC-content DNA sequence. Therefore, the DNA scope of their editing ability is complementary to each other. In the rice protoplast system, the ability of LbCpf1 to cut and then induce the chromosome fragment to duplicate, i.e. to create a new HPPD gene was tested, as shown in the FIG. 31.

With reference to Example 1, the pHUE411 vector (https://www.addgene.org/62203/) was used as the backbone, and the sgRNA expression cassette was removed by restriction enzyme digestion. The SV40 NLS-LbCpf1-nucleoplasmin NLS gene fragment synthesized in GenScript Biotechnology Company (Nanjing, China) replaced the Cas9 CDS of pHUE411. At 338 kb downstream of HPPD gene is a high-expression Ubi2 gene with a same expression orientation. Thus, a duplication strategy was used to increase the expression of HPPD, which confers resistance to HPPD inhibitor herbicides. To this end, acrRNA was designed in the upstream of the start codon of rice HPPD gene: 5′accccccaccaccaactcctccc3′, and the second crRNA was designed in the upstream of the start codon of rice UBI2 gene: 5′ctatctgtgtgaagattattgcc3′. A tandem crRNA sequence was synthesized with HH ribozyme and HDV ribozyme recognition sites at both ends, as shown below: 5′AAATTACTGATGAGTCCGTGAGGACGAAACGAGTAAGCTCGTCTAATTTCTACT AAGTGTAGATaccccccaccaccaactcctcccTAATTTCTACTAAGTGTAGATctatctgtgtgaagatt attgccTAATTTCTACTAAGTGTAGATGGCCGGCATGGTCCCAGCCTCCTCGCTGGCG CCGGCTGGGCAACATGCTTCGGCATGGCGAATGGGAC3′. It was connected to the end of LbCpf1 protein expression cassette in the vector according to the operating instructions of the Seamless Cloning Kit from HB-infusion Hanbio Biotechnology Co. Ltd. (Shanghai, China). The maize UBI1 promoter was used to drive both Lbcpf1 protein and crRNA in the same expression cassette. This vector was named pQY2658.

With reference to Example 1, plasmids with high-purity and high-concentration were prepared for PEG-mediated transformation of rice protoplasts. After 48-72 hr of transformation protoplast DNA was extracted for detecting duplication-editing events. The primers from both sides of the targets were designed to cover the duplicated area, and the target fragment was expected to be 494 bp. The primer sequences are:

Ubi2pro-Primer 5:
gtagcttgtgcgtttcgatttg
HPPDcds-Primer 10:
tcgacgtggtggaacgcgag

The PCR amplification of the DNA extracted from the pQY2658 transformed rice protoplasts for the duplicated adapter fragments did produce bands with the expected size, and the sequencing result of the amplicon is consistent with the expected chromosome fragment duplicated adapter sequence. The sequencing result is shown in SEQ.No. 27.

The test results on protoplast transformed with pQY2658 proved that LbCpf1 nuclease can effectively cleave the target, generate the detectable duplication of chromosome fragments between the targeted cut sites. It shows that the present invention can be used to create new genes through the duplication, inversion, or translocation of chromosome fragments, which can also be realized on the nuclease system of Cas12a.

### Example 16: OsCATC Gene Connected to the Chloroplast Signal Peptide Domain Through Deletion of a Chromosome Segment

Three genes of rice, namely glycolate oxidase OsGLO3, oxalate oxidase OsOXO3 and catalase OsCATC, form a photorespiratory branch, which was referred to as GOC branch. The glycolic acid produced by photorespiration could be directly catalyzed into oxalic acid in chloroplast and finally completely decomposed into CO₂ by introducing the GOC branch into rice by transgene and locating it in the chloroplast, thereby creating a photosynthetic CO₂ concentration mechanism similar to C4 plants, which helped improve the photosynthetic efficiency and yield of rice (Shen et al. Engineering a New Chloroplastic Photorespiratory Bypass to Increase Photosynthetic Efficiency and Productivity in Rice. Molecular Plant, 2019, 12(2): 199-214).

By using the method presented by the invention, the protein domains of different genes could be recombined by non-transgenic method to add chloroplast signal domains to genes that required chloroplast localization. Primer OsCATC-sgRNA1: 5′gtcctggaacaccgccgcgg3′ was designed at the end of the chloroplast signal peptide domain of LOC4331514 gene of upstream 28 Kb of OsCATC gene; OsCATC-sgRNA2:5′atcagccatggatccctaca3′ was designed in the first five amino acid coding regions of OsCATC gene. The chloroplast signal peptide domain of LOC4331514 gene was expected to fuse with the coding region of OsCATC gene to produce a new CATC gene located in chloroplast after the removal of inter-target fragment. Dual-target editing vector pQY2654 was constructed by the method stated in Example 1, and the primers used were

OsCATC-sgRNA1-For2654F:
taggtctccggcggtcctggaacaccgccgcggGT
TTAAGAGCTATGCTGGAAACAGC,
and
OsCATC-sgRNA2-For2654R:
taggtctccaaactgtagggatccatggctgatgc
ttcttggtgccgcg.

High-purity and high-concentration plasmids were prepared for PEG-mediated transformation of rice protoplast; the protoplast DNA was extracted for detection of deletion edit event; detection primers were designed to extend the linker segment after the middle 28 Kb chromosome segment deletion for the targets on both sides; sequencing was performed, and the primer sequences were shown below:

| | |
|---|---|
| OsCATC-TestF: | ccacaaaacgagtggctcag |
| OsCATC-TestR: | gtgagcgagttgttgttgttcc |
| OsCATC-seqF: | ctcttccctccactccactg |

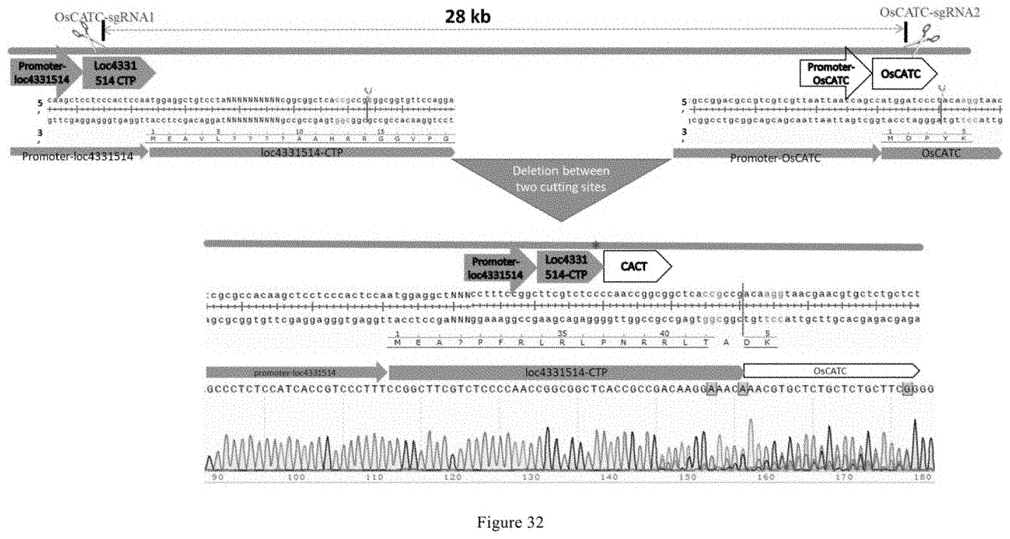

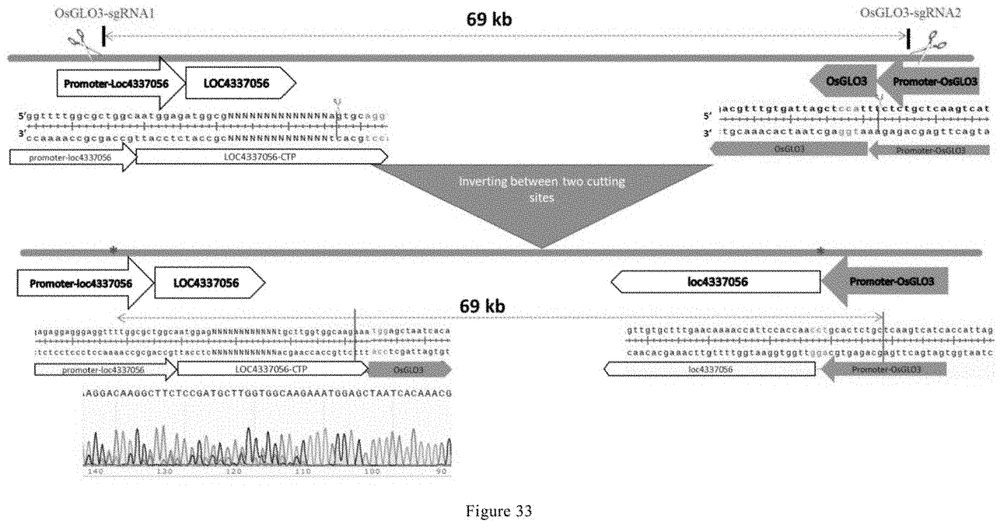

The test result was shown in FIG. 32. The extraction of DNA from pQY2654 transformed rice protoplast could detect chromosome fragment deletion event through PCR amplification of the expected junction region after the targeted deletion, and then sequencing; the chloroplast signal peptide domain of LOC4331514 gene was fused with the coding region of OsCATC gene to create a new gene; the sequencing result was shown in SEQ ID No: 28. The protoplast test result of pQY2654 indicates that new genes combined from new protein domains could be created through the deletion of chromosome segments between different protein domains by using the method of the present invention.

### Example 17: OsGLO3 Gene Connected to the Chloroplast Signal Peptide Domain Through Inversion of a Chromosome Segment

As stated in Example 16, the OsGlO3 gene also needed to be heterotopically expressed in chloroplasts to improve the photosynthetic efficiency of rice. Hence, for the OsGLO3 gene, OsGLO3-gRNA1:5′gtcctggaacaccgccgcgg3′ was designed at the end of chloroplast signal peptide domain of the LOC4337056 gene of the upstream 69 Kb, and OsGLO3-sgRNA2:5′tgatgacttgagcagagaaa3′ was designed in the initiation codon region of the OsCATC gene; the chloroplast signal peptide domain of the LOC4337056 gene was expected to fuse with the coding region of OsGLO3 gene to produce the new GLO gene located in chloroplast after the inversion of inter-target fragments. Dual-target editing vector pQY2655 was constructed as described in Example 1 using primers OsGLO3-sgRNA1-For2655F:taggtctccggcgcgatgcttggtggcaagtgcGTTTAAGAGCTATGCT GGAAACAGC and OsGLO3-sgRNA2-For2655R:taggtctccaaactttctctgctcaagtcatcagcttcttggtgccgcg. High-purity and high-concentration plasmids were prepared for PEG-mediated transformation of rice protoplast; the protoplast DNA was extracted for detection of inversion edit event; detection primers were designed to extend the linker segment after the middle 69 Kb chromosome segment inversion for the targets on both sides; sequencing was performed, and the primer sequences were shown below:

| | |
|---|---|
| OsGLO3-TestF1: | cctccttgttcgtgttctccg |
| OsGLO3-TestF2: | cggtcggttggttcatttcagg |
| OsGLO3-TestR1: | catccagcagtgtgctaccag |
| OsGLO3-TestR2: | cttgagaaggcctccctgttc |

The test result was shown in FIG. 33. The extraction of DNA from pQY2655 transformed rice protoplast could detect chromosome fragment inversion event through PCR amplification of the expected junction region after the targeted inversion, and then sequencing; the chloroplast signal peptide domain of LOC4337056 gene was fused with the coding region of OsCATC gene to create a new gene; the sequencing result was shown in SEQ ID NO: 29 The protoplast test result of pQY2655 indicated that new genes combined from new protein domains could be created through the inversion of chromosome segments between different protein domains by using the method of the present invention.

### Example 18: Creation of Herbicide-Resistant Rice Through Knock-Up of Endogenous PPO2 Gene Expression

The rice PPO2 gene was located on rice chromosome 4; bioinformatics analysis indicated that the S-adenosylmethionine decarboxylase (hereinafter referred as “SAMDC”) gene was approx. 436 kb downstream the PPO2 gene; the PPO2 gene and SAMDC gene had the same transcription direction on the chromosome. According to the analysis performed with the rice gene expression profile data (http://rice.plantbiology.msu.edu/index.shtml) from the International Rice Genome Sequencing Project, the expression intensity of SAMDC gene in rice leaves was tens to hundreds of times that of PPO2 gene; the promoter of SAMDC gene was a strong and constitutive express promoter.

For the rice PPO2 gene, the genomic DNA sequence of rice PPO2 and SAMDC was entered into the CRISPOR online tool (http://crispor.tefor.net/) respectively, to seek available edit targets following the procedures stated in Examples 1 and 2. Based on the online scoring, the following targets were selected for test between the promoter and CDS region of PPO2 and SAMDC genes:

| | |
|---|---|
| PPO2-guide RNA1 | gatttacttgttgtcttgtg |
| PPO2-guide RNA2 | ttggggctcttggatagcta |
| SAMDC-guide RNA1 | ggttggtcagaacactgtgc |
| SAMDC-guide RNA2 | actgtgccggagatggagga |

PPO2-guide RNA1 and PPO2-guide RNA2 were close to the initiation codon ATG of PPO2 between the promoter and CDS region of PPO2 gene, (i.e. 5′UTR); SAMDC-guide RNA1 and SAMDC-guide RNA2 were also close to the SAMDC protein initiation codon between SAMDC gene promoter and CDS region (i.e. 5′UTR).

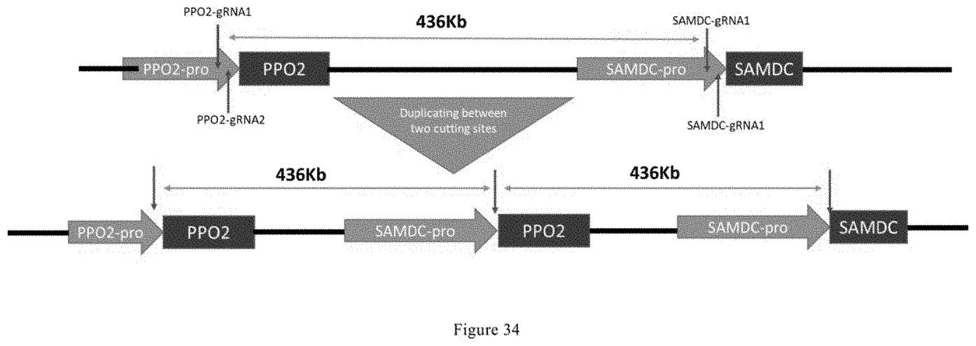

The following primers were designed for above-noted targets; dual-target edit vectors pQY1386 and pQY1387 were constructed, and the edit event of chromosome fragment duplication between two targeted cuts was expected to be achieved; the novel gene expressed by PPO2 CDS driven by SAMDC promoter was produced at the duplication fragment linker, as shown in FIG. 34.

| | DNA sequence |
|---|---|
| Primer ID | (5′ to 3′) |
| PPO2- | taggtctccggcggat |
| esgRNA1-F | ttacttgttgtcttgt |
| | gGTTTAAGAGCTATGC |
| | TGGAAACAGC |
| PPO2- | taggtctccggcgttg |
| esgRNA2-F | gggctcttggatagct |
| | aGTTTAAGAGCTATGC |
| | TGGAAACAGC |
| SAMDC- | taggtctccaaacgca |
| esgRNA1-R | cagtgttctgaccaac |
| | cgcttcttggtgccgc |
| | g |
| SAMDC- | taggtctccaaactcc |
| esgRNA2-R | tccatctccggcacag |
| | tgcttcttggtgccgc |
| | g |

Wherein,

pQY1386 contains the combination of PPO2-guide RNA1 and SAMDC-guide RNA1

pQY1387 contains the combination of PPO2-guide RNA2 and SAMDC-guide RNA2.

Vector plasmids were extracted, and agrobacterium strain EHA105 was electrotransformed. Agrobacterium tumefaciens-mediated transformation was performed with rice variety Jinjing 818 as the receptor by the method stated in Example 2. Several rounds of callus identification were conducted during the transformation and selection, and positive calli of duplication events were selected for differentiation.

The detection primers in the table below were used to amplify the fragments containing target sites on both sides or the fragments produced from the fusion of predicted SAMDC promoter and PPO2 coding region; the length of PCR product was expected to be 300-1000 bp; the primer5-F+primer4-R combination was used to detect the fusion segment at the intermediate linker after chromosome fragment duplication; the predicted product length was 912 bp.

| | Sequence |
|---|---|
| Primer ID | (5′ to 3′) |
| OsPPO2duplicated- | tctcggacaaa |
| primer1-F | cagtgcaccc |
| OsPPO2duplicated- | caaattgtggg |
| primer2-F | ccgtatgcacg |
| OsPPO2duplicated- | gcttcctcagc |
| primer3-R | ctgtacgcc |
| OsPPO2duplicated- | acccgccctcg |
| primer4-R | gagttgg |
| OsPPO2duplicated- | gtgcagtaagt |
| primer5-F | ggatgtactaa |
| | tggagtc |
| OsPPO2duplicated- | gccggaggcgt |
| primer6-F | gaagaagttc |
| | ca |
| OsPPO2duplicated- | gacacaatggt |
| primer7-R | gcaccgtgc |
| OsPPO2duplicated- | ggactcagaga |
| primer8-R | ggacataggag |
| | tc |

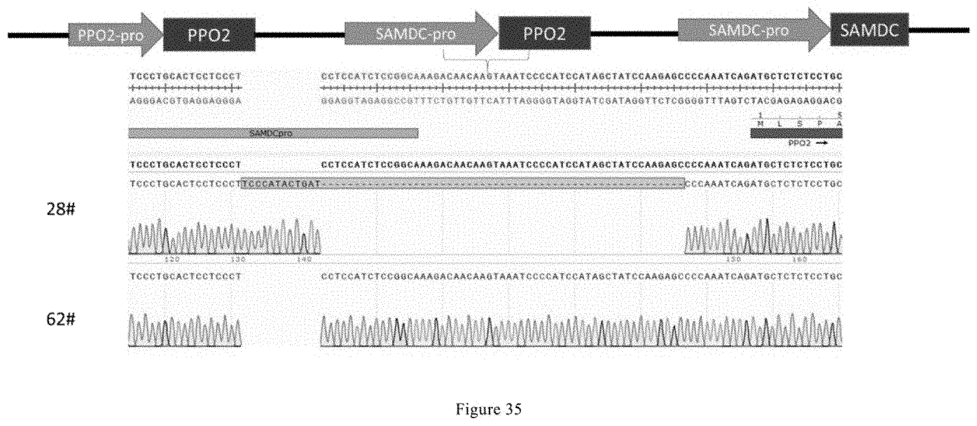

According to the final identification result, duplication edit events were detected in QY1386/818-28 # and QY1386/818-62 # calli; the sequencing result at the duplication fragment linker was shown in SEQ ID NO: 30 and SEQ ID NO: 31; The sequence alignment result was shown in FIG. 35; the result at #62 callus linker was exactly in line with expectations; seamless connection was observed, but duplication edit event was not detected in later differentiated seedlings.

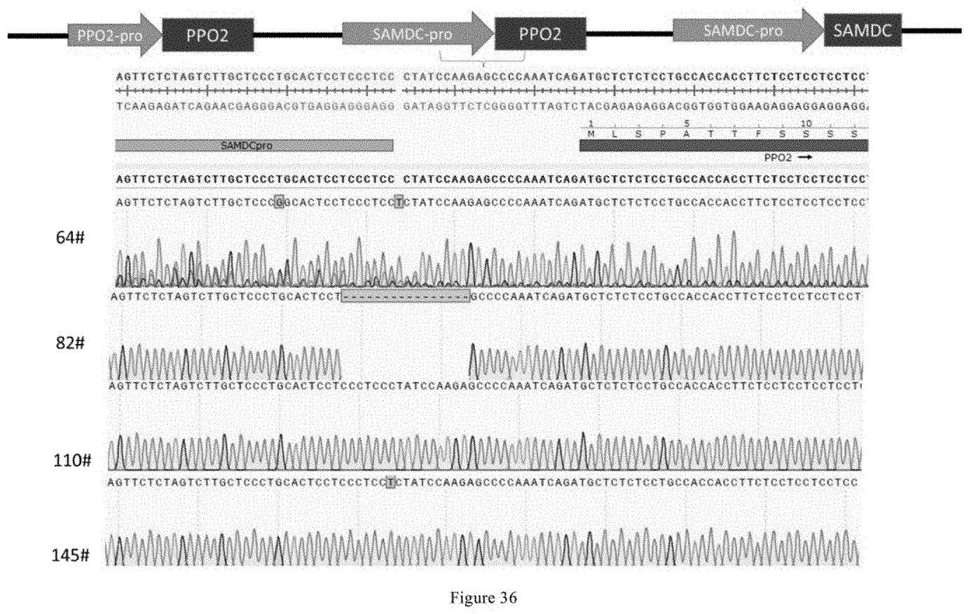

Five duplication edit events were detected in the calli of QY1837; the sequencing results at the duplication fragment linker were shown in SEQ ID NO: 32, SEQ ID NO: 33, SEQ ID NO: 34, SEQ ID NO: 35, and SEQ ID NO: 36; some sequence alignment results were shown in FIG. 36.

Duplication events were detected in QY1837 differentiated seedlings; the results of the PCR amplified products and the sequencing at chromosome duplication linkers, PPO2 targets and SAMDC targets of some T0 seedlings were given below:

| T0 seedling | Genotype at | | |
|---|---|---|---|
| No. | duplication point | PPO2 target | SAMDC target |
| 1387/818-2 | Heterozygosis: | +T, −11 bp | −6 bp |
| | Seamless, −2 bp | | |
| 1387/818-4/6/7 | Heterozygosis: | +T, −11 bp | −2 bp |
| | Seamless, −2 bp | | |
| 1387/818-36 | No duplication | Heterozygosis, | Heterozygosis, |
| | detected | doublet | doublet |
| 1387/818-38 | Heterozygosis: | Heterozygosis, | Heterozygosis, |
| | Seamless, −2 bp | doublet | doublet |

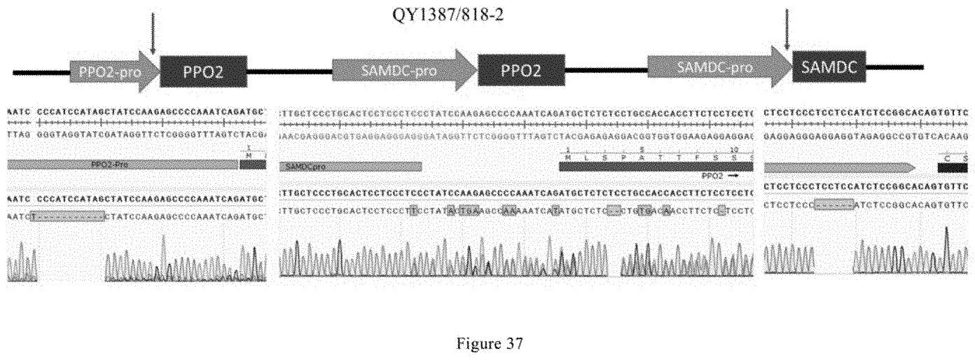

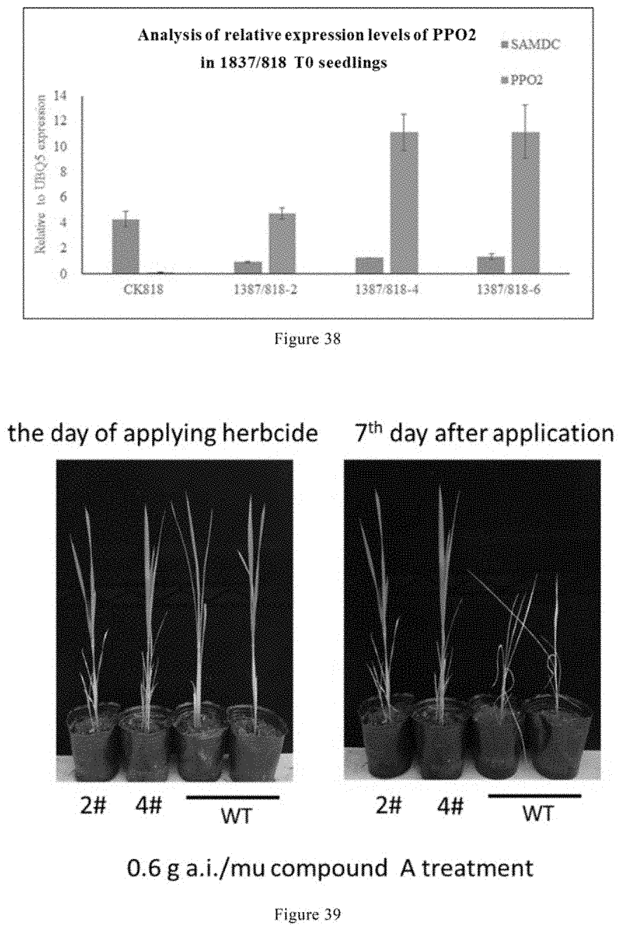

The result of comparison of 1387/818-2 with the sequencing peak diagram was shown in FIG. 37; it was obvious that novel PPO2 gene expressed by PPO2 driven by SAMDC promoter developed in the genome; small fragments were deleted on both sides of the target, but this did not affect the integrity of CDS region reading frame.

Quantitative PCR detection of the relative expression of PPO2 gene was performed for TO-generation differentiated seedlings 1387/818-2, 1387/818-4 and 1387/818-6; the experiment operation was in line with Example 2; the quantitative PCR primer sequence was 5′-3′ as follows:

| | |
|---|---|
| UBQ5-F | ACCACTTCGACCGCCACTACT |
| UBQ5-R | ACGCCTAAGCCTGCTGGTT |
| RT-OsPP02-F | GTATGGCTCTGTCATTGCTGGTG |
| RT-OsPPO2-R | GTTTATTCCTTCCTTTCCCTGGC |
| RT-OsSAMDC-F | ACCTATGGTTACCCTTGAAATGTG |
| RT-OsSAMDC-R | CTGGGATAATGTCAGAGATGCC |

With UBQ5 as the internal control, the result was shown in FIG. 35; compared with the wild-type rice Jinjing 818 control, the PPO2 gene expression of double-edited seedlings increases significantly, while SAMDC expression decreases relatively.

The herbicide resistance of T0 seedlings of 1387/818-2 and 1387/818-4 was preliminarily determined as stated in Example 6; wild-type Jinjing 818 seedlings with similar plant heights were taken as the control, and compound A was applied to them and TO seedlings at the same time at a chemical concentration of 0.6 g a.i./mu; the culture temperature was kept at 28° C. on a 16 (light)+8 (dark) basis; pictures were taken to record the results 7 days after application, as shown in FIG. 36. The T0 seedlings of 1387/818-2 and 1387/818-4 were a little dried-up at the top, while a few drug spots appear on the leaf surface; the wild-type Jinjing 818 withered and died; the result indicated that the SAMDC promoter-driven high expression of PPO2 protein enables rice to resist PPO inhibitor herbicides.

### Example 19: Creation of Herbicide-Resistant Rice Through Knock-Up Expression of the Endogenous OsPPO2 Gene Caused by CRISPR/Cas9 Targeted Chromosome Cutting and Inversion after Agrobacterium-Mediated Transformation

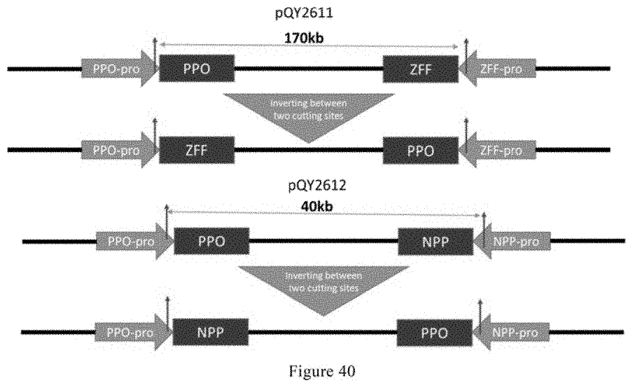

With reference to Example 4 to operate OsPPO2 gene, OsZFF (LOC_OS04G41560), a highly expressed gene at 170 kb downstream from OsPPO2 in the opposite direction, was selected to design two sgRNAs targeting in the regions close to the protein start codons ATGs, and a dual-target editing vector pQY2611 was constructed. Similarly, to increase the inversion probability, the downstream 40 kb from OsPPO2 and highly expressed gene OsNPP (LOC_OS04G41340) in the opposite direction of OsPPO2 was also selected to design another two sgRNAs targeting in the regions close to the protein start codons ATGs, and another dual-target editing vector pQY2612 was constructed. The three selected targets were shown as the following table. It was expected that the editing could produce double-strand DNA cut and then the inversion of chromosome fragments between the targets to form a new gene with high expression of PPO2, respectively, as shown in FIG. 40:

| | |
|---|---|
| OsPPO2-guide RNA2 | ttggggctcttggatagcta |
| 560-guide RNA3 | agttagtttagtcgtctcga |
| 340-guide RNA4 | tccggtggegtctgtttggt |

The following primers were used to construct the vectors:

| Primer ID | Sequence (5′ to 3′) |
|---|---|
| OsPPO2- | taggtctccggcgttggggctctt |
| sgRNA2-F | ggatagctaGTTTAAGAGCTATGC |
| | TGGAAACAGC |
| 560-sgRNA3-R | taggtctccaaactcgagacgact |
| | aaactaactgcttcttggtgccgc |
| | g |
| 340-sgRNA4-R | taggtctccaaacaccaaacagac |
| | gcaagacaagcttcttggtgccgc |
| | g |

Wherein,

pQY2611 contains the combination of OsPPO2-guide RNA2 and 560-guide RNA3

pQY2612 contains the combination of OsPPO2-guide RNA2 and 340-guide RNA4

pQY2611 contains the combination of OsPO2-guide RNA2 and 560-guide RNA3

pQY2612 contains the combination of OsPO2-guide RNA2 and 340-guide RNA4

The vector plasmid was extracted and transformed into Agrobacterium tumefacien strain EHA105. The rice variety Jinjing 818 was used as the receptor for Agrobacterium-mediated transformation, and the transformation method was referred to Example 2. Several rounds of callus identification were carried out during the transformation-post selection process, and the callus with positive inversion events was selected for differentiation.

The detecting primers in the table below were used to amplify the fragments containing both target sites and the fused fragment between the predicted 560 promoter and the PPO2 coding region. The length of the PCR amplicon was expected 300-1000 bp. Primer2-F+Primer12-R and Primer3-F+Primer10-R were used to detect fused fragments at the junction of OsZFF after chromosome fragmentation and then inversion, and the expected amplicon lengths were 512 bp and 561 bp, respectively. Similarly, Primer2-F+Primer6-R and Primer3-F+Primer7-R were used to detect the fused fragments at the junction of OSNPP after chromosome fragmentation and then inversion, and the expected amplicon lengths were 383 bp and 666 bp, respectively.

| | Sequence |
|---|---|
| Primer ID | (5′ to 3′) |
| OsPPO2 inverted- | caaattgtgggc |
| primer2-F | cgtatgcacg |
| OsPPO2 inverted- | cacgtctccact |
| primer12-R | ctcccagcc |
| OsPPO2 inverted- | gcttcctcagc |
| primer3-F | ctgtacgcc |
| OsPPO2 inverted- | Gcccgtgcagc |
| primer10-R | ctagccatc |
| OsPPO2inverted- | ccacctccccg |
| primer6-R | gcggtactg |
| OsPPO2inverted- | gatatgccgga |
| primer7-R | ccggacatgt |

The pQY2611-transformed calli were identified through PCR and amplicon sequencing. 292 samples were identified, 19 of which were positive for the inversion. The identified inversion event genotypes were shown as following table:

| | Junction sequence between | Junction sequence between |
|---|---|---|
| Positive | inverted PPO2 coding region | inverted ZFF coding region |
| callus ID | and ZFF promoter region | and PPO2 promoter region |
| 2611/818-3 | −4 bp, homozygous | +T, homozygous |
| 2611/818-10 | seamless, homozygous | no identification |
| 2611/818-13 | −2 bp, homozygous | −1 bp, homozygous |
| 2611/818-21 | seamless, homozygous | +T, homozygous |
| 2611/818-24 | seamless, homozygous | not detect |
| 2611/818-53 | −26 bp, | not detect |
| | messychromatogrampeaks | |
| 2611/818-54 | −30 bp, | not detect |
| | messychromatogrampeaks | |
| 2611/818-55 | −26 bp, | not detect |
| | messychromatogrampeaks | |
| 2611/818-67 | −30 bp, homozygous | not detect |
| 2611/818-83 | seamless, homozygous | +T, messychromatogrampeaks |
| 2611/818-85 | seamless, homozygous | not detect |
| 2611/818-90 | +418 bp, homozygous | not detect |
| 2611/818-92 | −2 bp, homozygous | +T, messychromatogrampeaks |
| 2611/818-102 | seamless, homozygous | +T, messychromatogrampeaks |
| 2611/818-106 | seamless, homozygous | +T, messychromatogrampeaks |
| 2611/818-107 | −2 bp, homozygous | +T, messychromatogrampeaks |
| 2611/818-108 | −2 bp, heterozygous | not detect |
| 2611/818-109 | heterozygous | not detect |
| 2611/818-121 | −22 bp, homozygous | not detect |

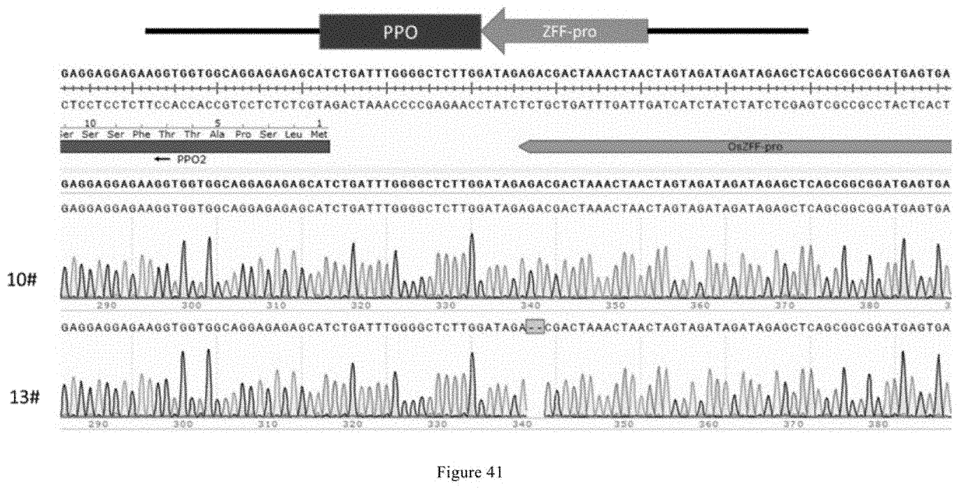

The sequencing results of the OsZFF promoter fused with OsPPe2 CDS region were shown in Seq No. 37, Seq No. 38, Seq No. 39, Seq No. 40, Seq No. 41, Seq No. 42, Seq No. 43. The alignment comparison results of 2611/818-10 and 2611/818-13 chromatogram peaks are shown in FIG. 41. The events 2611/818-3, 2611/818-10, 2611/818-54 were differentiated further and obtained inversion positive T0 plants.

Similarly, pQY2612-transformed calli were identified for inversion events. A total of 577 callus samples were identified, and 45 callus samples were detected to be positive for the inversion. The genotypes of inversion events detected were shown as following table:

| Positive | | genotype of inverted |
|---|---|---|
| callus ID | genotype of inverted PPO2 | NFF |
| 2612/818-5 | seamless, homozygous | −1 A, homozygous |
| 2612/818-29 | seamless, homozygous | not detect |
| 2612/818-34 | −3 bp, homozygous | not detect |
| 2612/818-62 | seamless, homozygous | not detect |
| 2612/818-64 | seamless, homozygous | not detect |
| 2612/818-66 | +1 T, homozygous | not detect |
| 2612/818-129 | seamless, homozygous | Seamless, homozygous |
| 2612/818-156 | seamless, homozygous | Seamless, homozygous |
| 2612/818-157 | seamless, homozygous | Seamless, homozygous |
| 2612/818-366 | seamless, homozygous | −5 bp |
| 2612/818-377 | −31 bp, the start codon is broken, | not detect |
| | homozygous | |
| 2612/818-419 | seamless, homozygous | +1 bp T |
| 2612/818-444 | Seamless, homozygous | Seamless, homozygous |
| 2612/818-457 | Seamless, homozygous | Seamless, homozygous |
| 2612/818-497 | +1 T, homozygous | −3 bp, homozygous |

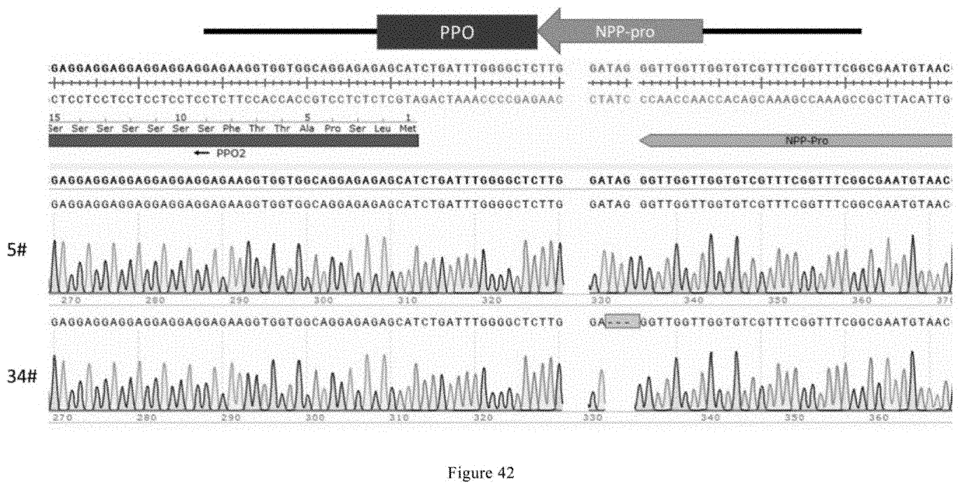

The sequencing results of the OsNPP promoter fused OSPPO2 CDS region were shown in Seq No. 44, Seq No. 45, Seq No. 46, and Seq No. 47. The sequencing results of events 2612/818-5 and 2611/818-34 and the chromatogram peaks are shown in FIG. 42. Eventually only event 2612/818-29 was differentiated successfully and obtained a positive T0 plant with desired inversion.

### Example 20: Test on Creation of Novel PPO2 Gene with Maize Protoplasts

As shown in Example 7, the gene distribution on chromosomes was collinear among different plants; the method for successful creation of novel genes like EPSPS, PPO1, PPO2 and HPPD in new mode of expression was versatile among other plant species. According to Example 18, novel PPO2 gene was created through the PPO2 gene selection in maize and the duplication of chromosome fragments between maize SAMDC genes, and then dual-target edit vectors were constructed for maize protoplast test.

The following targets were selected for test between the promoter and CDS region of PPO2 and SAMDC genes: ZmPPO2-sgRNA1:5′ggatttgcttgttgtcgtgg3′ was close to the initiation codon ATG of PPO2 protein between the promoter and CDS region of PPO2 gene (i.e. 5′UTR). ZmSAMDC-sgRNA2:5′gtcgattatcaggaagcagc3′ and ZmSAMDC-sgRNA3:5′acaatgctggagatggaggg3′ were close to the SAMDC protein initiation codon ATG between the promoter and CDS region of SAMDC gene (i.e. 5′UTR).

Dual-target edit vectors pQY1340 and pQY1341 were constructed using the following primers designed for above-noted targets.

| Primer ID | DNA sequence (5′ to 3′) |
|---|---|
| ZmPP02-sgRNA1-F | Taggtctccggcgggatttgctt |
| | gttgtcgtggGTTTAAGAGCTAT |
| | GCTGGAAACAGC |
| ZmSAMDC-sgRNA2-R | Taggtctccaaacgtcgattatc |
| | aggaagcagctgcaccagccggg |
| | aatcgaac |
| ZmSAMDC-sgRNA3-R | Taggtctccaaacacaatgctgg |
| | agatggagggtgcaccagccggg |
| | aatcgaac |

Wherein, pQY1340 contained ZmPPO2-sgRNA1 and SAMDC-sgRNA2 targets combination, while pQY1341 contained ZmPPO2-sgRNA1 and SAMDC-sgRNA3 targets combination.

High-concentration plasmids were prepared for above-noted vectors and used for the protoplast transformation in maize following the procedures stated in Example 1 for preparation and transformation of rice protoplasts; it was slightly different from rice in that a vacuum degree of 15 pa should be maintained for 30 minutes before enzymolysis so that the enzymatic hydrolysate contacts the cells more adequately; the maize variety used was B73.

The detection primers in the table below were used to amplify the fragments containing target sites on both sides or the fragments produced from the fusion of predicted SAMDC promoter and PPO2 coding region; the length of PCR product was expected to be 300-1100 bp; the ZmSAMDC test-F1+ZmPPO2 test-R2 combination was used to detect the fusion segment at intermediate linker after chromosome fragment duplication; the expected product length was approx. 597 bp; inner primer ZmSAMDC test-F2 was used for sequencing.

| Primer ID | DNA sequence (5′ to 3′) |
|---|---|
| ZmSAMDC test-F1 | gggtggcaaaaagtctagcag |
| ZmSAMDC test-R1 | ggtgagcaggagcttggtag |
| ZmSAMDC test-F2 | cggaggcgtgaagaagttccag |
| ZmSAMDC test-R2 | ccgtgcaagatccagaacagag |
| ZmPP02 test-F1 | gccatcctgagacctgtagc |
| ZmPP02 test-R1 | gcacaagggcataaagcaccac |
| ZmPP02 test-F2 | gcagtccgaccatacccatacc |
| ZmPP02 test-R2 | cctcgaaggcacaaacacgtac |

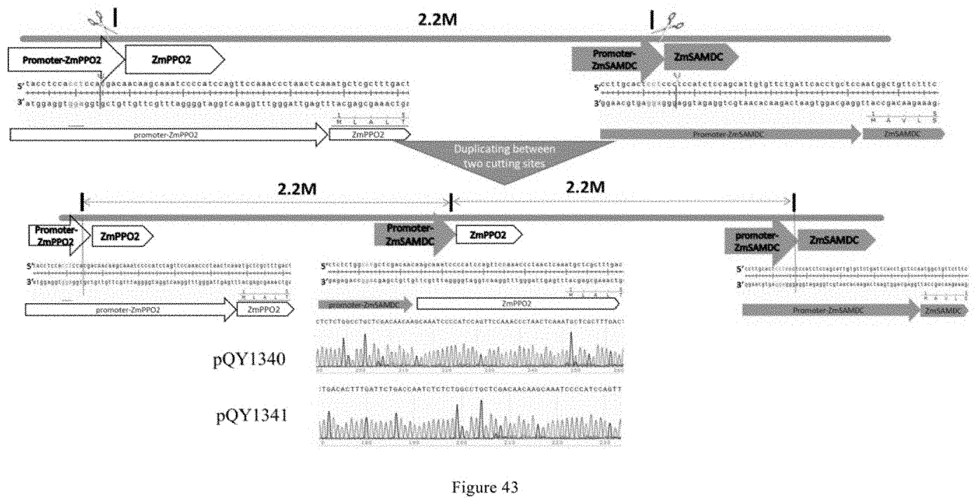

1% agarose gel electrophoresis test was performed for the PCR reaction product, and the result indicated that the predicted positive band (approx. 597 bp) into which the ZmSAMDC promoter and ZmPPO2 coding region were fused was detected in all pQY1340 and pQY1341 transformed maize protoplast samples. Positive fragments were sequenced, and the PPO2 duplication event sequencing result of pQY1340 vector transformed protoplast test was shown in SEQ ID NO: 48; the PPO2 duplication event sequencing result of pQY1341 vector transformed protoplast test was shown in SEQ ID NO: 49. The result of comparison with the sequence at predicted chromosome segment duplication linker was shown in FIG. 43, indicating that the SAMDC gene promoter and the PPO2 gene expression region can be linked up directly to create novel PPO2 gene with strong promoter-driven expression; thus, it's obvious that the method provided by the present invention for creating novel genes was also applicable to maize.

### Example 21: Creation of Novel PPO2 Gene in Wheat Protoplast Test

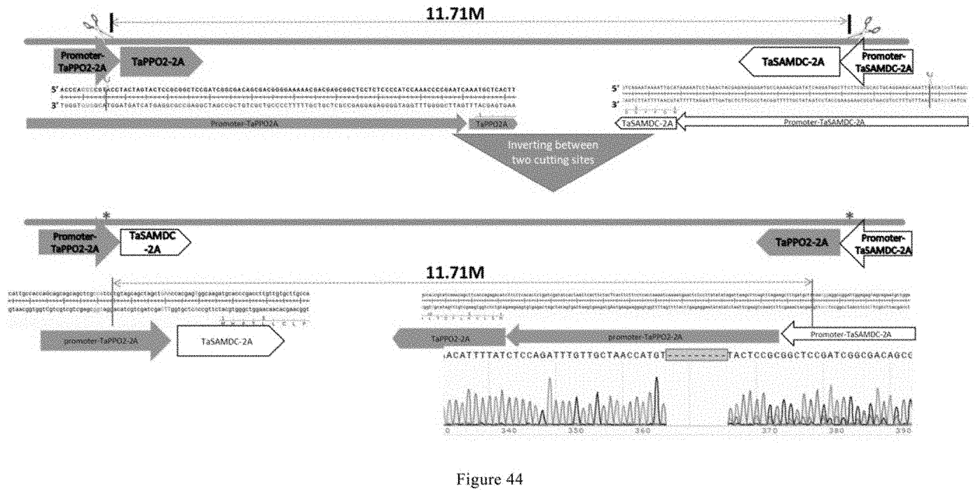

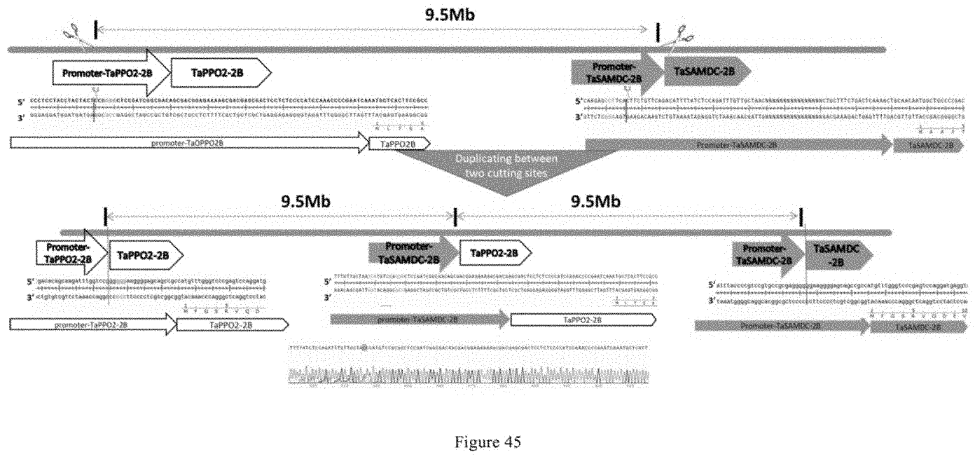

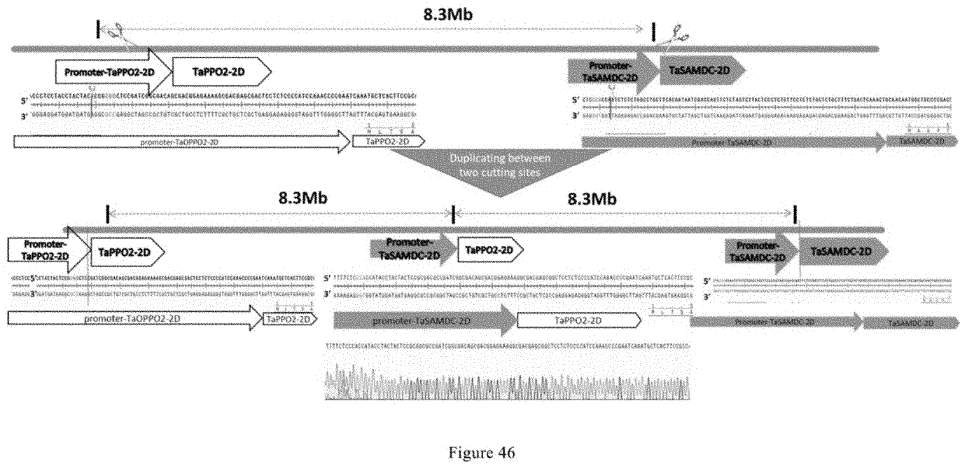

According to Example 18, in wheat the chromosome fragment region between PPO2 gene and SAMDC gene was selected for dual-target editing to create the novel gene expressed by PPO2 coding region driven by the SAMDC promoter. Wheat was hexaploid, so there were 3 sets of PPO2 genes and SAMDC genes in genomes A, B, and D. The TaPPO2-2A (TraesCS2A02G347900) gene was located at the wheat 2A chromosome, and the TaSAMDC-2A (TraesCS2A02G355400) gene was approx. 11.71 Mb downstream; since the TaSAMDC-2A and TaPPO2-2A gene transcriptions are in opposite directions on the same chromosome, it's necessary to choose inversion editing strategy, as shown in FIG. 44; TaPPO2-2B (TraesCS2B02G366300) was located at the wheat 2B chromosome, and TaSAMDC-2B (TraesCS2B02G372900) was 9.5 Mb downstream; since TaSAMDC-2B and TaPPO2-2B gene expressions are in the same direction on the chromosome, the duplication editing strategy should be used, as shown in FIG. 45; TaPPO2-2D (TraesCS2D02G346200) was located at the wheat 2D chromosome, and TaSAMDC-2D (TraesCS2D02G352900) was 8.3 Mb downstream; since the TaSAMDC-2D and TaPPO2-2D gene transcriptions are in the same direction on the chromosome, the duplication edit event should be selected, as shown in FIG. 46.

The DNA sequence of wheat ABD gene group PPO2 and SAMDC gene was entered into the CRISPOR online tool (http://crispor.tefor.net/) respectively, to seek available edit targets. Based on the online scoring, the following targets were selected for test between the promoter and CDS region of PPO2 and SAMDC genes:

| Primer ID | DNA sequence (5′ to 3′) |
|---|---|
| 2A guide RNA1 | GCGGAGTACTAGTAGGTACG |
| 2A guide RNA2 | TGTGAATTTGTTTCCTGCAG |
| 2A guide RNA3 | ATGACGCAGAGCACTCGTCG |
| 2A guide RNA4 | CTTCTCGTAGTTTAGGATTT |
| 2B guide RNA1 | CCCTCCTACCTACTACTCCG |
| 2Bguide RNA2 | TGTGACATTTTTTTCATCTT |
| 2Bguide RNA3 | CGAAGGCGACGACGGAGAGC |
| 2Bguide RNA4 | TCACTTCTGTTCAGACATTT |
| 2Dguide RNA1 | CCGCGGAGTAGTAGGTAGCA |
| 2Dguide RNA2 | GCTTCACGATAATCGACCAG |
| 2Dguide RNA3 | CGATGACGCCGACGCAGAGC |
| 2Dguide RNA4 | CCAATCTCTCTGGCCTGCTT |

2A guide RNA1 and 2A guide RNA2 were close to the initiation codon of PPO2 protein between the promoter and CDS region of PPO2 gene (i.e. 5′UTR); 2A guide RNA3 and 2A guide RNA4 were close to the SAMDC protein initiation codon between SAMDC gene promoter and CDS region (i.e. 5′UTR). 2B and 2D followed the same principle as above.

The following primers were designed for above-noted targets to construct the vector with pHUE411 vector (https://www.addgene.org/62203/) as the framework using the method presented in “Xing H L, Dong L, Wang Z P, Zhang H Y, Han C Y, Liu B, Wang X C, Chen Q J. A CRISPR/Cas9 toolkit for multiplex genome editing in plants. BMC Plant Biol. 2014 Nov. 29; 14(1):327”.

| Primer ID | DNA sequence (5′ to 3′) |
|---|---|
| TaPPO2A-T2 for | taggtctccggcgGCGGAGTACTAGTAGGT |
| 2626/2627BsaIF | ACGGTTTAAGAGCTATGCTGGAAACAGC |
| TaSAMDCA-for | taggtctccaaacTGTGAATTTGTTTCCTG |
| 2627/2629BsaIR | CAGgcttcttggtgccgcg |
| TaPPO2A-T2 for | taggtctccggcgATGACGCAGAGCACTCG |
| 2628/2629BsaIF | TCGGTTTAAGAGCTATGCTGGAAACAGC |
| TaSAMDCA-for | taggtctccaaacCTTCTCGTAGTTTAGGA |
| 2626/2628BsaIR | TTTgcttcttggtgccgcg |
| TaPPO2B-T2 for | taggtctccggcgCCCTCCTACCTACTACT |
| 2630/263 IBsaIF | CCGGTTTAAGAGCTATGCTGGAAACAGC |
| TaSAMDCB for | taggtctccaaacTGTGACATTTTTTTCAT |
| 2630/2632BsaIR | CTTgcttcttggtgccgcg |
| TaPPO2B for | taggtctccggcgCGAAGGCGACGACGGAG |
| 2632/2633BsaIF | AGCGTTTAAGAGCTATGCTGGAAACAGC |
| TaSAMDCB for | taggtctccaaacTCACTTCTGTTCAGACA |
| 2631/2633BsaIR | TTTgcttcttggtgccgcg |
| TaPP02D for | taggtctccggcgCCGCGGAGTAGTAGGTA |
| 2635/2636BsaIF | GCAGTTTAAGAGCTATGCTGGAAACAGC |
| TaSAMDCD for | taggtctccaaacGCTTCACGATAATCGAC |
| 2634/2636BsaIR | CAGgcttcttggtgccgcg |
| TaPP02D for | taggtctccggcgCGATGACGCCGACGCAG |
| 2636/2637BsaIF | AGCGTTTAAGAGCTATGCTGGAAACAGC |
| TaSAMDCD for | taggtctccaaacCCAATCTCTCTGGCCTG |
| 2635/2637BsaIR | CTTgcttcttggtgccgcg |

The following dual-target combined gene edit vectors were constructed using the method described in the literature above. To be more specific, pCBC-MT1T2 plasmid (https://www.addgene.org/50593/) was used as template to amplify dual-target fragments sgRNA1+3, sgRNA1+4, sgRNA2+3 and sgRNA2+4 to construct the sgRNA expression cassettes. BsaI digests the pHUE411 vector framework, and the gel was recovered; the target fragment was used for ligation reaction directly after digestion. T4 DNA ligase was used to link up the vector framework and target fragment, and the ligation product was transformed to the Trans5α competent cell; different monoclonal sequences were selected; after the sequences were confirmed by sequencing to be correct, the Sigitech small-amount high-purity plasmid extraction kit was used to extract plasmids and attain recombinant plasmids, which were respectively named as pQY2626, pQY2627, pQY2628, pQY2629, pQY2630, pQY2631, pQY2632, pQY2633, pQY2634, pQY2635, pQY2636, and pQY2637 as follows:

pQY2626 contains the combination of 2A-guide RNA1 and 2A-guide RNA3

pQY2627 contains the combination of 2A-guide RNA1 and 2A-guide RNA4

pQY2628 contains the combination of 2A-guide RNA2 and 2A-guide RNA3

pQY2629 contains the combination of 2A-guide RNA2 and 2A-guide RNA4

pQY2630 contains the combination of 2B-guide RNA1 and 2B-guide RNA3

pQY2631 contains the combination of 2B-guide RNA1 and 2B-guide RNA4

pQY2632 contains the combination of 2B-guide RNA2 and 2B-guide RNA3

pQY2633 contains the combination of 2B-guide RNA2 and 2B-guide RNA4

pQY2634 contains the combination of 2D-guide RNA1 and 2D-guide RNA3

pQY2635 contains the combination of 2D-guide RNA1 and 2D-guide RNA4

pQY2636 contains the combination of 2D-guide RNA2 and 2D-guide RNA3

pQY2637 contains the combination of 2D-guide RNA2 and 2D-guide RNA4

High-concentration plasmids were prepared for above-noted vectors and used for the protoplast transformation in wheat following the procedures stated in Example 1 for preparation and transformation of rice protoplasts; it's slightly different from rice that a vacuum degree of 15 pa should be maintained for 30 minutes before enzymolysis so that the enzymatic hydrolysate contacts the cells more adequately. The variety of wheat used was KN199; the seeds were from the Teaching and Research Office on Weeds, School of Plant Protection, China Agricultural University, and were propagated at our lab; the wheat seeds were sown in small pots for dark culture at 26° C. for approx. 10 d-15 d; stems and leaves of the etiolated seedlings were used to prepare protoplasts.

The detection primers in the table below were used to PCR amplify the fragments containing target sites on both sides or the fragments produced from the fusion of predicted SAMDC promoter and PPO2 coding region; the length of PCR product was expected to be 300-1100 bp; the ZmSAMDC test-F1+ZmPPO2 test-R2 combination was used to detect the fusion segment at intermediate linker after chromosome fragment duplication; the PCR product length was expected to be 300-1100 bp; primer pair combinations TaSAMDCA-g600F&TaPPO2A+g480R, TaSAMDCB-g610F &TaPPO2B+g470R, and TaSAMDCD-g510F &TaPPO2D+g490R were respectively used to test the fusion segments at intermediate linker after the chromosome fragment duplication or inversion in the ABD genome; the product length was expected to be approx. 1 kb.

| Primer ID | DNA sequence (5′ to 3′) |
|---|---|
| TaPPO2A-g330F | TCACCAAAAATGTGTGCGCTCGTG |
| TaPPO2A+g480R | ACACAGGTCGCACCATTCGCTCCAACAC |
| TaPPO2B-g360F | CACATTCACCAAAAATGTGTGTGCTCGACTG |
| TaPPO2B+g470R | AGGTCGCACCATTCGCCACAATCC |
| TaPPO2D-g340F | TGGGTCCGTTTTTTATTGGGCGCTCAAG |
| TaPPO2D+g490R | CTCAATTCGCTCCAGCATTCGCCG |
| TaSAMDCA+g670R | CAGACCTCCATCTCGGGAATGATGTCG |
| TaSAMDCA-g600F | TCCGTATGGCGCTTGTTCGTTGTTCG |
| TaSAMDCB+g620R | AGCACAGGAGACATGGCCATCAGCAG |
| TaSAMDCB-g610F | GAATTTGCCGTGGCTTATGGCATCATG |
| TaSAMDCD+g670R | CCTCCATCTCAGGGATAATGTCAGAGATT |
| TaSAMDCD-g510F | TACAGCATTCCGTCCCTGCTGTGAC |

1% agarose gel electrophoresis test was performed for the PCR reaction product, and the result indicated that the predicted SAMDC promoter and the positive strip/band of approx. 1 kb in the PPO2 coding region fusion segment can be detected in the pQY2626 and PQY2627 transformed samples of the 2A genome, the pQY2630 and pQY2631 transformed samples of 2B genome, and the QY2634, pQY2635 and pQY2636 transformed samples of 2B genome.

PCR amplified positive fragments were sequenced, and the PPO2 inversion event sequencing result of pQY2626 vector transformed protoplast test was shown in SEQ ID NO: 50; the PPO2 inversion event sequencing result of PQY2627 vector transformed protoplast test was shown in SEQ ID NO: 51. The result of sequence comparison at inversion linker of predicted chromosome segment indicated that the TaSAMDC-2A gene promoter and the TaPPO2-2A gene expression region can be linked up directly to create novel PPO2 gene with strong promoter-driven expression.

The PPO2 duplication event sequencing result of pQY2630 vector transformed protoplast test was shown in SEQ ID NO: 52; the PPO2 duplication event sequencing result of pQY2631 vector transformed protoplast test was shown in SEQ ID NO: 53. The result of sequence comparison at duplication linker of predicted chromosome segment indicated that the TaSAMDC-2B gene promoter and the TaPPO2-2B gene expression region can be linked up directly to create novel PPO2 gene with strong promoter-driven expression; the result of pQY2631 sequencing peak diagram comparison was shown in FIG. 45.

The PPO2 duplication event sequencing result of pQY2634 vector transformed protoplast test was shown in SEQ ID NO: 54; the PPO2 duplication event sequencing result of pQY2635 vector transformed protoplast test was shown in SEQ ID NO: 55. The PPO2 duplication event sequencing result of QY2636 vector transformed protoplast test was shown in SEQ ID NO: 56. The comparison with the predicted sequence at chromosome segment duplication linker indicated that TaSAMDC-2D gene promoter and TaPPO2-2D gene expression region can be linked up directly to create novel PPO2 gene with strong promoter-driven expression; the result of pQY2635 sequencing peak diagram comparison was shown in FIG. 46.

According to the results of these protoplast tests, novel PPO2 genes expressed by TaPPO2 driven by TaSAMDC promoter can also be created through chromosome segment inversion or duplication in wheat; therefore, it's obvious that the method presented in the present invention for creating new genes was also applicable to wheat.

### Example 22: Creation of Herbicide-Resistant Rape with Knock-Up Endogenous PPO2 Gene Expression Through Agrobacterium Tumefaciens-Mediated Transformation

Brassica napus was tetraploid, where the chromosome set was AACC; the redundancy between the A and C genomes enables the creation of new genes with different combinations of gene elements through the deletion or rearrangement of chromosome segments. To create a rape germplasm resistant to PPO inhibitor herbicides, the up-regulation of endogenous PPO gene expression was a feasible technical route. The analysis of the genomic data of rape C9 chromosome shows that the 30S ribosomal protein S13 gene (hereinafter referred as 30SR) was located at approx. 23 kb upstream the BnC9.PPO2; both were in the same direction for transcription on the same chromosome; the expression levels of rape 30SR and BnC9.PPO2 in various tissues in rapeseed were analyzed with Brassica EDB database (https://brassica.biodb.org/); 30SR and BnC9.PPO2 were principally expressed in leaves, and the expression level of 30SR was significantly higher than that of BnC9.PPO2; the PPO2 protein expression level was expected to rise when the novel gene expressed by BnC9.PPO2 CDS driven by 30SR promoter was created by deleting the chromosome segment between 30SR promoter and BnC9.PPO2 CDS; in that way, rape gained herbicide tolerance.

Targets available were identified by finding the information on C9 chromosome of transformed receptor rape variety Westar at the rape database website (http://cbi.hzau.edu.cn/bnapus/) a total of 6 targets were selected:

| | |
|---|---|
| BnC9.PPO2-guide RNA1 | TTCCTGTATCCTTCTTCAG |
| BnC9.PPO2 -guide RNA2 | AAGATGAGAGCTACGGATA |
| BnC9.PPO2 -guide RNA3 | AACCCAACAGAAACGCGTC |
| BnC9.PPO2 -guide RNA4 | CGAAAGAGAAGTAGACCAG |
| BnC9.PPO2 -guide RNA5 | CTCCTGAAACGACAACAAA |
| BnC9.PPO2 -guide RNA6 | CTTAAGTTATGTTTCTAAC |

Wherein, guide RNA1, guide RNA2 and guide RNA3 were close to the initiation codon ATG of 30SR protein between the promoter and CDS region of 30SR gene (i.e. 5′UTR region); guide RNA4, guide RNA5 and guide RNA6 were close to the BnC9.PPO2 protein initiation codon ATG between BnC9.PPO2 gene promoter and CDS region (i.e. 5′UTR region).

With reference to Example 1, the edit vectors of different target combinations, namely pQY2533, pQY2534, pQY2535 and pQY2536 were constructed with pHSE401 vector as the framework; where:

pQY2533 contains the combination of guide RNA1 and guide RNA4

pQY2534 contains the combination of guide RNA2 and guide RNA5

pQY2535 contains the combination of guide RNA3 and guide RNA6

pQY2536 contains the combination of guide RNA1 and guide RNA5

Vector plasmids were extracted, and agrobacterium strain GV3101 was electrotransformed. Agrobacterium tumefaciens-mediated transformation was performed with rape variety Westar as receptor using the method below:

\textcircled{1} Sowing: Seeds were soaked in 75% alcohol for 1 min, disinfected with 10% sodium hypochlorite solution for 9 min, washed 5 times with sterile water, sown into M0 medium, and cultured in darkness at 24° C. for 5-6 days.

\textcircled{2} Preparation of agrobacterium: 3 mL of liquid LB medium was transferred into the sterile tube; the solution with agrobacterium was subjected to shake culture in a 200-rpm shaker at 28° C. for 20-24 h. The solution with bacteria was incubated for 6-7 h in the LB culture medium. The cultured bacteria solution was poured into a 50 mL sterile centrifuge tube; the tube was centrifuged for 5 min at 6000 rpm; the supernatant was discarded, and a moderate amount of DM suspension was added; the solution was shaken well and the OD600 value of infecting bacteria solution was set to approx. 0.6-0.8.

\textcircled{3} Infection and co-cultivation of explants: The prepared infecting bacteria solution was activated on ice, while the hypocotyls of seedlings cultured in darkness were cut off vertically with sterile forceps and scalpel; the cut-off explant was infected for 12 minutes in a dish, which was shaken every 6 min during infection; the explants were transferred to sterile filter paper after infection, and the excess infection solution was sucked out; then, the explants were placed in M1 culture medium and co-cultured at 24° C. for 48 h.

\textcircled{4} Callus induction: After the co-culture, the explants were transferred to M2 culture medium, where callus was induced for 18-20 days; the culture conditions: Light culture at 22-24° C.; light for 16 hrs/dark for 8 hrs. The conditions for differentiation culture and rooting culture were the same as the present stage.

\textcircled{5} Induced germination: The callus was transferred to M3 culture medium for differentiation culture, and succession was performed every 14 days until germination.

\textcircled{6} Rooting culture and transplantation: After the buds were differentiated to see obvious growth points, the buds were carefully cut off from the callus with sterile forceps and scalpel; the excess callus was removed as much as possible, and then the buds were transferred to M4 medium for rootage. Rooted plants were transplanted into the culture soil; T1-generation seeds were achieved through bagged selfing of the TO-generation regenerated plants.

The formula of culture medium used during the process was as follows:

Sowing Culture Medium M0

| Culture | Chemical | | |
|---|---|---|---|
| medium | name | Dosage | Method of preparation |
| M0 | MS | 2.22 g | Dissolved in 1000 mL |
| | Agar | 8 g | of double distilled |
| | | | water; pH adjusted to |
| | | | 5.8-5.9; autoclaved |

DM Transform Buffer Solution

| Culture | | | | |
|---|---|---|---|---|
| medium | Chemical name | Dosage | | Method of preparation |
| DM | MS | 4.43 | g | Dissolved in 1000 mL |
| | Sucrose | 30 | g | of double distilled |
| | 2,4-D | 1 | mL | water; pH adjusted to |
| | Kinetin (KT) | 1 | mL | 5.8-5.9; AS added |
| | Acetosyringone (AS) | 1 | mL | after autoclaving |

Co-Culture Medium M1

| Culture | | | | |
|---|---|---|---|---|
| medium | Chemical name | Dosage | | Method of preparation |
| M1 | MS | 4.43 | g | Dissolved in 1000 mL |
| | Sucrose | 30 | g | of double distilled |
| | Manitol | 18 | g | water; pH adjusted to |
| | 2,4-D | 1 | mL | 5.8-5.9; AS added |
| | Kinetin (KT) | 1 | mL | after autoclaving |
| | Phytagel | 4-5 | g | |
| | Acetosyringone (AS) | 1 | mL | |

Screening Medium M2

| Culture | | | | |
|---|---|---|---|---|
| medium | Chemical name | Dosage | | Method of preparation |
| M2 | MS | 4.43 | g | Dissolved in 1000 mL |
| | Sucrose | 30 | g | of double distilled |
| | Manitol | 18 | g | water; pH adjusted to |
| | 2,4-D | 1 | mL | 5.8-5.9; silver nitrate, |
| | Kinetin (KT) | 1 | mL | timentin and |
| | Phytagel | 4-5 | g | hygromycin added |
| | AgNO₃ (silver nitrate) | 0.2 | mL | after autoclaving |
| | Timentin | 1 | mL | |
| | Hygromycin (Hyg) | 0.2 | mL | |
| | Acetosyringone (AS) | 1 | mL | |

Differential Medium M3

| Culture | | | | |
|---|---|---|---|---|
| medium | Chemical name | Dosage | | Method of preparation |
| M3 | MS | 4.43 | g | Dissolved in 1000 mL of |
| | Glucose | 10 | g | double distilled water; pH |
| | Xylose | 0.25 | g | adjusted to 5.8-5.9; ZT, |
| | MES | 0.6 | g | IAA, timentin and |
| | Phytagel | 4-5 | g | hygromycin added after |
| | Zeatin (ZT) | 1 | mL | autoclaving |
| | Indoleacetic acid (IAA) | 0.2 | mL | |
| | Timentin | 1 | mL | |
| | Hygromycin (Hyg) | 0.2 | mL | |

Rooting Medium M4

| Culture | | | |
|---|---|---|---|
| medium | Chemical name | Dosage | Method of preparation |
| M4 | MS | 2.2 g | Dissolved in 1000 mL of |
| | Sucrose | 10 g | double distilled water; pH |
| | Indolebutyric acid | 1 mL | adjusted to 5.8-5.9; timentin |
| | (IBA) | | added after autoclaving |
| | Agar | 10 g | |
| | Timentin | 0.5 mL | |

After the emergence of seedlings, leaves were taken from T0 seedlings for molecular identification. The detection primers in the table below were used to amplify the fragments containing target sites on both sides or the fragments produced from the fusion of predicted 30SR promoter and BnC9.PPO2 coding region; where klenow fragment was removed, the PCR product length should be approx. 700 bp.

| Primer ID | Sequence (5′ to 3′) |
|---|---|
| 30SR PRO-F: | TGACTTTGCATCTCGCCACT |
| PP02 PRO-R3: | GCAGATGATGATGATGATAAGCTC |

363 T0 seedlings from the transformation of the four vectors were tested; Klenow fragment deletion event was observed in 18 plants; the probability of Klenow fragment deletion varied depending on target combination; even the same target combination may bring about different probabilities of Klenow fragment deletion; pQY2534 vector offered the highest probability (10.96%), while pQY2535 vector offered the lowest probability (2%); the average probability was on the order of 5.56%.

Analysis of the sequencing result of 18 individual plants with positive knocked out: The sequencing results of 10 individual plants showed seamless Klenow fragment deletion between two targets; homozygous seamless knockout occurred in QY2533/w-7, and heterozygous knockout occurred in the other 9 plants; compared with the expected sequence after deletion, the insertion or deletion of small fragments of base was observed in 8 individual plants; up to 32 bases were deleted in the 30SR promoter region, and this was not expected to affect the promoter activity; homozygous knockout was observed in QY2533/w-36, QY2533/w-42, QY2535/w-32 and QY2536/w-124; the details of result were as follows.

| Plant No. | PCR test result | Sequencing result analysis |
|---|---|---|
| QY2533/W-7 | With strip/band | Deletion; seamless; |
| | | homozygous |
| QY2533/W-36 | With strip/band | Deletion; −2 bp; homozygous |
| QY2533/W-39 | With strip/band | Deletion; −13 bp; heterozygous |
| QY2533/W-42 | With strip/band | Deletion; +1 bp; T; |
| | | homozygous |
| QY2534/W-32 | With strip/band | Deletion; seamless; |
| | | miscellaneous peaks |
| QY2534/W-36 | With strip/band | Deletion; seamless; |
| | | miscellaneous peaks |
| QY2534/W-40 | With strip/band | Deletion; seamless; |
| | | miscellaneous peaks |
| QY2534/W-44 | With strip/band | Deletion; −32 bp; miscellaneous |
| | | peaks |
| QY2534/W-53 | With strip/band | Deletion; seamless; |
| | | miscellaneous peaks |
| QY2534/W-55 | With strip/band | Deletion; seamless; |
| | | miscellaneous peaks |
| QY2534/W-56 | With strip/band | Deletion; seamless; |
| | | miscellaneous peaks |
| QY2534/W-59 | With strip/band | Deletion; seamless; |
| | | miscellaneous peaks |
| QY2535/W-32 | With strip/band | Deletion; +10 bp; homozygous |
| QY2535/W-46 | With strip/band | Deletion; −1 bp; heterozygous |
| QY2536/W-73 | With strip/band | Deletion; seamless; |
| | | miscellaneous peaks |
| QY2536/W-77 | With strip/band | Deletion; seamless; |
| | | miscellaneous peaks |
| QY2536/W-78 | With strip/band | Deletion; +1 bp; heterozygous |
| QY2536/W-124 | With strip/band | Deletion; +A; homozygous |

The sequencing result showed that the 30SR promoter can be directly connected with the BnC9.PPO2 CDS region to create novel PPO2 gene with strong promoter-driven expression after the deletion of inter-target sequence. The sequencing results of the 30SR promoter fused BnC9.PPO2 CDS region were shown in Seq No. 57, Seq No. 58, Seq No. 59, Seq No. 60, and Seq No. 61.

T0 seedling test result indicated that the method presented in the present invention enabled the creation of novel genes expressed by the BnC9.PPO2 CDS region driven by the 30SR promoter; so, it's obvious that the way presented in the present invention to create new genes was also applicable to rape. The results of test on rice, corn, wheat, Arabidopsis thaliana, and rape demonstrate that the method provided by the present invention was designed for purposeful precise creation of novel genes with combinations of different gene elements or different protein domains in both monocotyledons and dicotyledons.

### Example 23: Creation of Rice Blast Resistance Through Knock-Up Expression of an Endogenous Gene OsWAK1

OsWAK1 is a novel functional protein kinase. It was reported that overexpression of the OsWAK1 gene can confer resistance to rice blast (Li et al. A novel wall-associated receptor-like protein kinase gene, OsWAK1, plays important roles in rice blast disease resistance. Plant Mol Biol, 2009, 69: 337-346). The OsWAK1 gene locates on rice chromosome 1. Through bioinformatics analysis, it was found that LOC_Os01g044350 (hereinafter referred to as 44350) gene, which is highly expressed in rice, locates about 26 kb upstream of OsWAK1 gene, and the 44350 gene and the OsWAK1 gene are in the opposite direction on the chromosome. The 44350 gene promoter can be used for inversion to increase the expression of OsWAK1 gene. Similarly, BBTI12 (MSU ID: LOC_Os01g04050), which is highly expressed in rice, locates about 206 kb upstream of OsWAK1 gene, and the BBTI12 gene and the OsWAK1 gene are in the same direction on the chromosome. The BBTI12 gene promoter can be used for duplication to increase the expression of OsWAK1 gene.

Similarly, the dual-target combination OsWAK1ts2:5′TTCAGCTAGCTGCTACACAA 3′ and 44350ts2: 5′ TAGAAGCTTTGATGCTTGGA 3′, was used to construct the duplication editing vector pQY1085. The construction primers used were bsaI-OsWAK1 5′UTR ts2-F:

5′AATGGTCTCAggcATTCagctagctgctacacaaGTTTAAGAGCTATG
CTGGAAACAGCAT3′
and
bsaI-44350 5′UTRts2-R:
5′AATGGTCTCAAAACTCCAAGCATCAAAGCTTCTAgcttcttggtgccg
cgc 3′.

Similarly, the dual-target combination OsWAK1ts2: 5′ TTCAGCTAGCTGCTACACAA 3′ and BBTI12ts2: 5′ CAAGTAGAGGAAATAGCTCA 3′ was used to construct the duplication editing vector pQY1089. The construction primers used were bsaI-OsWAK1 5′UTR ts2-F:

5′AATGGTCTCAGGCATTCAGCTAGCTGCTACACAAGTTTAAGAGCTATG
CTGGAAACAGCAT3′
and
bsaI-BBTI12 5′UTRts2-R:
5′AATGGTCTCAAAACTGAGCTATTTCCTCTACTTGGCTTCTTGGTGCCG
CGC3′.

The above two plasmids were extracted to transform Agrobacterium sp. EHA105. The recipient rice variety Jinjing 818 was transformed through Agrobacterium-mediated transformation and the transformation method was referenced to Example 2. During the transformation process, genotype identification at the junction regions was performed on the rice calli, and the inversion or duplication event-positive calli were selected to enter the differentiation stage for regeneration of seedlings.

For pQY1085 transformed rice calli, the primer44350tsdet-F+primerOsWAK1tsdet-F combination was used to detect the fusion fragment at the middle joint after the inversion of the chromosome fragment, and the PCR product length was expected to be 713 bp.

| Primer ID | Sequences(5′ to 3′) |
|---|---|
| 44350tsdet-F | CGATCGATTCATCGAGAGGGCT |
| 44350tsdet-R | ATCACCAGCACGTTCCCCTC |
| OsWAK1TSDET-F | TTTTGTGTGCCGCGACGAATGAG |
| OsWAK1TSDET-R | CATAACGCTGTCGACAATTGACCTG |

For pQY1089 transformed rice calli, the primerOsWAK1tsdet-F+primerBBTI12tsdet-R combination was used to detect the fusion fragment at the middle joint after the duplication of the chromosome fragment, and the PCR product length was expected to be 837 bp.

| Primer ID | Sequences(5′ to 3′) |
|---|---|
| BBTI12tsdet-F | TTTTCTTTTGCAACAGCAGCAAAGATT |
| BBTI12tsdet-R | AGGGTACATCCTAGACGAGTCCAAG |
| OsWAK1tsdet-F | TTTTGTGTGCCGCGACGAATGAG |
| OsWAK1tsdet-R | CATAACGCTGTCGACAATTGACCTG |

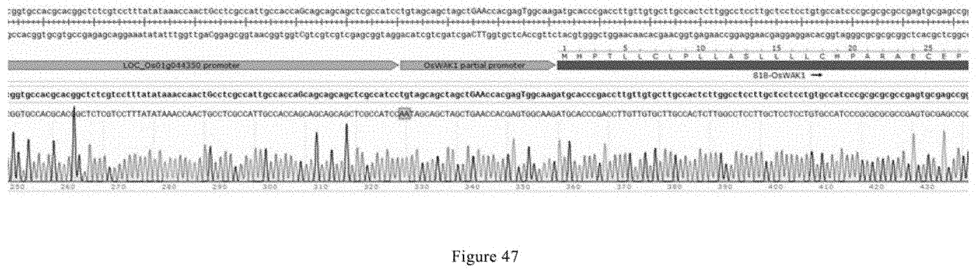

The above two vectors were referred to in Example 2 for Agrobacterium-mediated transformation of rice callus. After the callus was identified, the inversion or duplication event-positive calli were differentiated, and eventually positive edited seedlings were obtained. The results of molecular identification are shown in the FIG. 47. As shown, the pQY1085-transformed seedlings were detected to identify the inversion editing events in which the Os01g044350 promoter drives the OsWAK1 gene expression and thus a new OsWAK1 gene was formed. The representative sequences of the sequenced inversion events, QY1085/818-57, QY1085/818-107, QY1085/818-167, QY1085/818-23 are shown in SEQ ID NO: 62, SEQ ID NO: 64, SEQ ID NO: 65 and SEQ ID NO: 66.

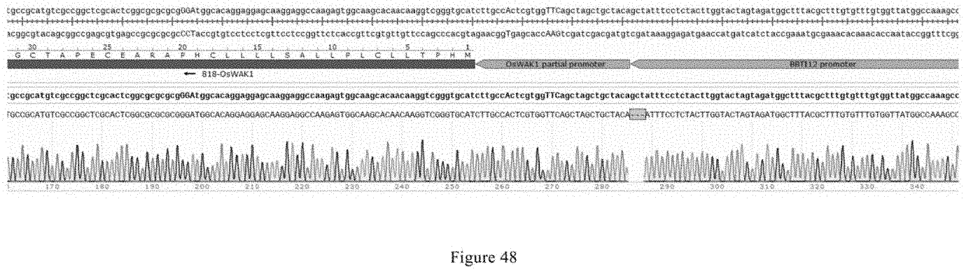

As shown in the FIG. 48, the pQY1089-transformed seedlings were detected to identify the duplication editing events in which the BBTI12 promoter drives the OsWAK1 gene expression and another new OsWAK1 gene was also formed. The representative sequences of the sequenced duplication events, QY1089/818-595, QY1089/818-321, QY1089/818-312 are shown in SEQ ID NO: 63, SEQ ID NO: 67 and SEQ ID NO: 68.

### Example 24: Creation of Blast-Resistant Rice Through Knock-Up Expression of Endogenous OsCNGC9 Gene in Rice

The cyclic nucleotide-gated channels (CNGCs) gene family encodes a set of non-specific, Ca²⁺ permeable cation channels. It was reported that overexpression of the OsCNGC9 gene can confer resistance to rice blast (Wang et al. A cyclic nucleotide-gated channel mediates cytoplasmic calcium elevation and disease resistance in rice. Cell Research, 2019, epub). The OsCNGC9 gene locates on rice chromosome 9. Through bioinformatics analysis, it was found that LOC_Os09g39180 (hereinafter referred to as 39180) gene, which is highly expressed in rice, locates about 314 kb downstream of OsCNGC9 gene, and the 39180 gene and the OsCNGC9 gene were in the opposite direction on the same chromosome. The 39180 gene promoter can be used for inversion to increase the expression of OsCNGC9 gene. In addition, LOC_Os09g39390 (hereinafter referred to as 39390), which is highly expressed in rice, locates about 456 kb downstream of OsCNGC9 gene, and the 39390 gene and the OsCNGC9 gene were in the same direction on the same chromosome. The 39390 gene promoter can be used for duplication to increase the expression of OsCNGC9 gene.

The dual-target combination OsCNGC9ts1: 5′ ACAGCAAGATTTGGTCCGGG 3′ and 39180ts1: 5′ ATGGAATGGAAGAGAATCGA 3′ was used to construct the inversion editing vector pQY1090. The construction primers used were bsaI-OsCNGC9 5′UTR ts1-F:

5′ AATGGTCTCAGGCAACAGCA
AGATTTGGTCCGGGGTTTAAGAGCTATGCTGGAAACAGCAT3′
and
bsaI-39180 5′UTRts1-R:
5′ AATGGTCTCAAAACTCGATTCTCTTCCATTCCATGCTTCTTG
GTGCCGCGC 3′.

The dual-target combination OsCNGC9ts1: 5′ ACAGCAAGATTTGGTCCGGG 3′ and 39390ts1: 5′ CTACTGGCCTCGATTCGTCG 3′ was used to construct the duplication editing vector pQY1094. The construction primers used were bsaI-OsCNGC9 5′UTR ts1-F:

5′ AATGGTCTCAGGCAACAGCA
AGATTTGGTCCGGGGTTTAAGAGCTATGCTGGAAACAGCAT3′
and
bsaI-39390 5′UTRts1-R:
5′ AATGGTCTCAAAACCGACGAATCGAGGCCAGTAGGCTTCT
TGGTGCCGCGC 3′.

The above two plasmids were extracted to transform Agrobacterium sp. EHA105. The recipient rice variety Jinjing 818 was transformed through Agrobacterium-mediated transformation and the transformation method was referenced to Example 2. During the transformation process, molecular identification was performed on the rice calli, and the inversion or duplication event-positive calli were selected to enter the differentiation stage and regeneration of seedlings.

For pQY1090 transformed calli, the primer39180tsdet-R+primerOsCNGC9tsdet-R combination was used to detect the fusion fragment at the middle joint after the inversion of the chromosome fragment, and the PCR product length was expected to be 778 bp.

| Primer ID | Sequences(5′ to 3′) |
|---|---|
| OsCNGC9tsdet-F | ACATCTCATGTGCAAGATCCTAGCA |
| OsCNGC9tsdet-R | AAACTGGTCCTGTCTCTCATCAGGA |
| 39180tsdet-F | TGGCTCAGCGAAGTCGAGC |
| 39180tsdet-R | CATGGTTGAACTGTCATGCTAATCAGT |

For pQY1094 transformed calli, the primer3390tsdet-F3+primerOsCNGC9tsdet-R combination was used to detect the fusion fragment at the middle joint after the duplication of the chromosome fragment, and the PCR product length was expected to be 895 bp.

| Primer ID | Sequences(5′ to 3′) |
|---|---|
| OsCNGC9tsdet-F | ACATCTCATGTGCAAGATCCTAGCA |
| OsCNGC9tsdet-R | AAACTGGTCCTGTCTCTCATCAGGA |
| 39390tsdet-F3 | TACTACAGCCTTTGCCTTTCACGGTTC |
| 39390tsdet-R | GTCTGCCACATGCCGTTGAG |

The above two vectors were referred to in Example 2 for Agrobacterium-mediated transformation of rice callus. After the callus was identified, the inversion or duplication event-positive calli were differentiated, and finally positive edited seedlings were obtained. Sequencing results prove that the pQY1090 transformed seedlings were detected to identify the inversion edited events in which the LOC_Os09g39180 promoter drives the OsCNGC9 gene expression and thus a new OsCNGC9 gene was formed. The representative sequences of the sequenced inversion events QY1090/818-192, QY1090/818-554 and QY1090/818-541, are shown in SEQ ID NO: 69, SEQ ID NO: 70 and SEQ ID NO: 71.

Sequencing results prove that the pQY1094 transformed seedlings were detected to identify duplication edited events in which the LOC_Os09g39390 promoter drives the OsCNGC9 gene expression and thus another new OsCNGC9 gene was also formed. The representative sequence of the sequenced duplicated event QY1094/818-202 are shown in SEQ ID NO: 72.

### Example 25: Pig IGF2 Gene Expression Knock-Up

IGF-2 (Insulin-like growth factor 2) is one of three protein hormones that have similar structure with insulin. IGF2 is secreted by the liver and circulates in the blood. It has the activity of promoting mitosis and regulating growth.

TNNI2 and TNNT3 encode muscle troponin I and troponin T, respectively, and they are the core components of muscle fibers. These two protein coding genes are constitutively and highly expressed in muscle tissue. Therefore, using the promoters of these two genes to drive the expression of IGF2 gene is expected to significantly increase its expression in muscle cells and promote growth. Since the directions of these two genes are opposite to IGF2 on the same chromosome, knock-up of IGF2 could be achieved by promoters exchange through chromosome segments inversion.

The experiment procedure was as follows:

1. CRISPR/Cas9 Target Site Selection and Vector Construction:

Using the CRISPR target online design tool (http://crispr.mit.edu/), we selected 20 bp sgRNA oligonucleotide sequences in the 5′UTR regions of pig IGF2, TNNI2, and TNNT3 genes respectively. The sgRNA oligos were synthesized by BGI, Qingdao.

| | |
|---|---|
| IGF2-sgRNA: | 5′ccgggtggaaccttcagcaa3′ |
| TNNI2-sgRNA: | 5′agtgctgctgcccagacggg3′ |
| TNNT3-sgRNA: | 5′acagtgggcacatccctgac3′ |

Diluting the synthesized sgRNA oligo with deionized water to 100 μmol/L in a reaction system (10 μL): positive strand oligo 1 μL, reverse strand oligo 1 μL, deionized water 8 μL. The annealing program of thermal cycler was set as follows: incubate at 37° C. for 30 min; incubate at 95° C. for 5 min, and then gradually reduce the temperature to 25° C. at a rate of 5° C./min. After annealing, the oligo was diluted by 250 volume using deionized water. pX459 plasmid was linearized with BbsI restriction endonuclease, the annealed product was ligated, and transformed into competent DH5α, a single colony was picked into a shaker tube, incubated at 37° C. for 12-16 h, 1 mL aliquot of bacterial solution was sent for sequencing. After sequencing verification, the bacterial solution was freezed and extracted for preparing the plasmid pX459-IGF2, pX459-TNNI2 and pX459-TNNT3. These plasmids were used for transfection in the following experiment.

2. Cell Transfection:

Thawing and culturing the pig primary fibroblast cell, removal of the culture medium and added preheated PBS for washing before transfection, then removed PBS and added 2 ml of 37° C. prewarmed trypsin solution. Digesting for 3 minutes in room temperature before terminating digestion. Suspending the cells in nucleofection solution, and diluting the volume to 10⁶/100 μl, adding plasmid to 5 μg/100 μl final concentration, performing electro-transformation with optimized program on the electroporator, adding 500 μl of preheated culture medium, and culturing the cell in a concentration of 20% FBS DMEM medium, at 37° C., with 5% carbon dioxide, and saturated humidity.

3. Cell Screening and Test:

When the cells reached 100% cell density, cells were lysed with NP40 buffer. Genomic DNA was extracted, and the target regions were amplified by PCR.

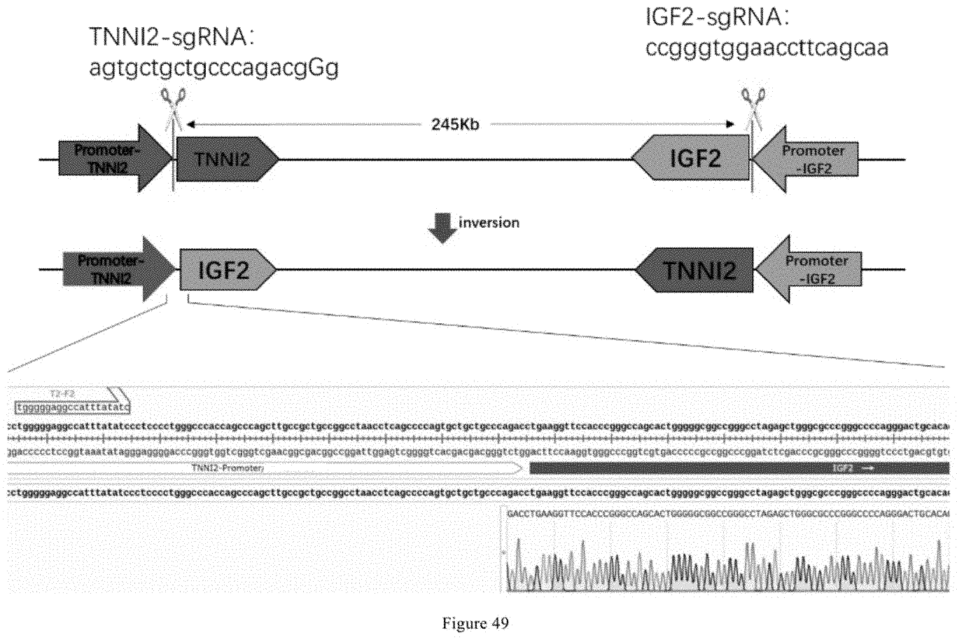

The result is shown in FIG. 49, using the primer pair (T2-F2: tgggggaggccatttatatc/IGF2-R2:acagctcgccactcatcc), the fusion events of the TNNI2 promoter and the IGF2 gene was successfully detected.

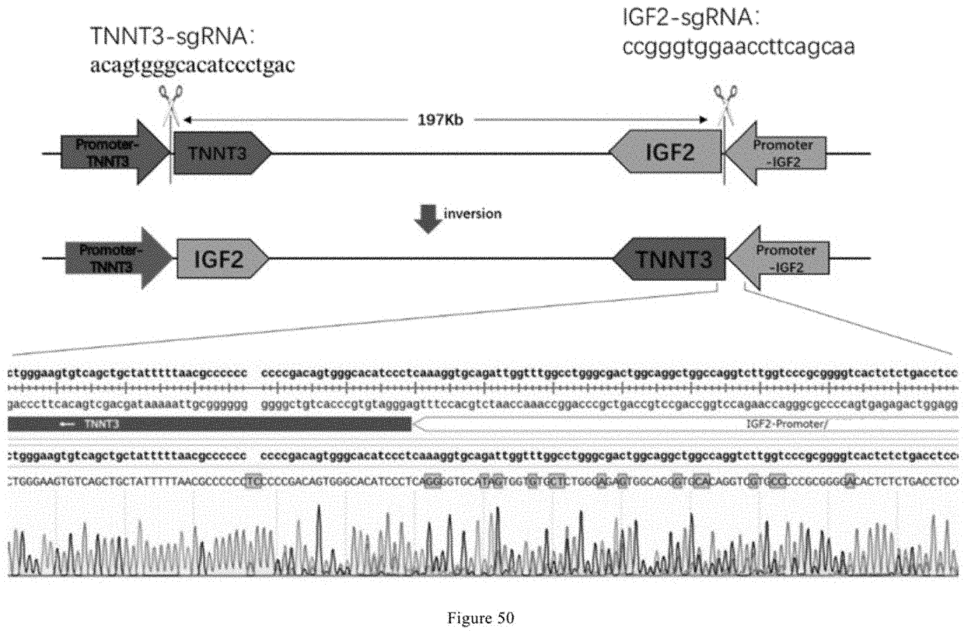

As showed in the FIG. 50, using the primer pair (TNNT3-R:CCCCAAGATGCTGTGCTTAG/IGF2-F:CTTGGGCACACAAAATAGCC), the fusion events of the IGF2 promoter and the TNNT3 gene were successfully detected. As affected by repeated sequences, efforts are still taken to detect the fusion events of the TNNT3 promoter and the IGF2 gene.

The invention fused the pig TNNI2 promoter with the IGF2protein coding region in vivo through the inversion editing events of the chromosome segment, which forms a new IGF2 gene with continuously high expression. These editing events created new fast-growing pig cell lines. This example shows that the method of the present invention can be used to create new genes in mammalian organisms.

### Example 26: Chicken IGF1 Expression Knock-Up

IGF1 (insulin like growth factor 1) is closely related to the growth and development of chickens. MYBPC1 (myosin binding protein C) is a highly expressed gene downstream of IGF1. A new gene with the MYBPC1 promoter driving IGF1 coding sequence was created through genome editing using a dual-target editing vector.

The experiment procedure was as follows:

1. CRISPR/Cas9 Target Site Selection and Targeted Cutting Vector Construction:

Using the CRISPR target online design tool (http://crispr.mit.edu/), 20 bp sgRNA oligonucleotide sequences were designed in the 5′UTR regions of chicken IGF1 gene and MYBPC1 gene respectively. sgRNA oligoes were synthesized by BGI. Diluting the synthesized sgRNA oligoes with deionized water to 100 μmol/L in a reaction system (10 μL): positive strand oligo 1 μL, reverse strand oligo 1 μL, deionized water 8 μL; the annealing program of thermal cycler was set as follows: incubate at 37° C. for 30 min; Incubate at 95° C. for 5 min, and then gradually reduce the temperature to 25° C. at a rate of 5° C./min; after annealing, the oligo was diluted by 250 volumes of deionized water. pX459 plasmid was linearized with BbsI restriction endonuclease, the annealed product was ligated, and transformed into competent DH5α, a single colony was picked into a shaker tube, incubated at 37° C. for 12-16 h, aliquot 1 mL of bacterial solution was sent for sequencing. After sequencing verification, the bacterial solution was freezed and extracted for preparing the plasmid pX459-IGF1 and pX459-MYBPC1, Those dual-target editing plasmids were used for transfection of chicken DF-1 cells.

2. Cell Culture and Passage of DF-1 Cells:

DF-1 (Douglas Foster-1) cell is chicken embryo fibroblast cell with vigorously proliferation ability, so DF-1 is the most popular cell line for in vitro study. DF-1 cells were thawed in a 37° C. water bath, and then inoculated in a petri dish and placed in a 37° C., 5% CO₂ constant temperature incubator for cell culture. The culture medium is 90% DMEM/F12+10% FBS. When the cell density reached more than 90%, passaging cell at a ratio of 1:2 or 1:3.

3. DF-1 Cell Transfection:

\textcircled{1} Preparing two 1.5 ml EP tubes and marked them as A tube and B tube respectively.

\textcircled{2} Placing 250 μl of Opti-MEM medium, 2.5 μg plasmid and 5 μl of P3000™ reagent in tube A.

\textcircled{3} Placing 250 μl of Opti-MEM medium and 3.75 μl of Lipofectamine® 3000 reagent in tube B.

\textcircled{4} Transferring the liquid from tube A to tube B with a pipette, and quickly mixing the liquid of tube A and tube B and vortexing for 10 seconds.

\textcircled{5} Vortexing AB tube mixture (liposome-DNA complex) and incubating at room temperature for 15 minutes.

\textcircled{6} Finally, slowly adding liposome-DNA complex to the DF-1 cell dish after the culture medium had been removed with pipette.

4. DF-1 Cell Screening and Test:

\textcircled{1} Culturing DF-1/PGCs cells, and the transfection efficiency is best when the confluence reaches 60-70%;

\textcircled{2} After 2 days of transfection, add 1 μl g/ml puromycin for screening;

\textcircled{3} After 4 days of transfection, replace with the fresh cell culture medium to remove puromycin, and continue to culture until the 7th day after transfection to increase the number of cells.

\textcircled{4} Collecting the cells and extracting cell DNA with Tiangen's Genomic DNA Kit according to the operating instructions.

\textcircled{5} Designing primers to amplify new gene fragments that are expected to be doubled or inverted.

The invention fused the chicken MYBPC1 promoter with the IGF1CDS region in vivo through the double editing events of the chromosome segment, which forms a new IGF1 gene with continuously high expression. These editing events created new fast-growing avian cell lines. This example shows that the method of the present invention can be used to create new genes in avian organisms.

### Example 27: Induced Gene Expression Through Chromosomal Segment Inversion in Yeast

FPP is a key precursor of many compounds in yeast. However, it can be degraded by many metabolic pathways in yeast, which affects the final yield of exogenous products such as terpenoids. The synthesis of squalene using FPP as substrate, is the first step of the ergosterol metabolic pathway, which is catalyzed by the squalene synthase encoded by the ERG9 gene. However, direct knockout of ERG9 gene would lead to the inability of yeast cells to grow, so the expression level of squalene synthase could only be regulated specifically, so that it could accumulate intracellular FPP concentration as well as maintaining its own growth. HXT promoter is a weakly glucose-responsive promoter, whose expression strength decreases with the decrease of glucose concentration in the external environment, which is consistent with the sugar metabolism process in the fermentation process, so it is an ideal inducible promoter.

As found in the Saccharomyces cerevisiae genome database website (https://www.yeastgenome.org/), both the HXT1 and ERG9 genes are located at the long arm end of chromosome VIII and are transcribed in the opposite direction, so the endogenous ERG9 gene promoter in yeast can be replaced by the HXT1 promoter, whose expression strength is responsive to glucose concentration, through the inversion editing events of the chromosome segment. It is expected that the specific induction of ERG9 gene expression will achieve the purpose of accumulation of FPP in yeast.

1. Vector Design and Construction

Vector design includes Cas9 vector and gRNA vector, which are constructed into two different backbones. For the Cas9 vector, we used pUC19 backbone, driven by yeast TEFl promoter, Cas9 sequence is yeast codon-optimized; gRNA vector used pUC57 backbone, SNR52 promoter and SUP4 terminator. The sgRNA is designed using an online tool (http://crispor.tefor.net/) and selected the following targets between the promoter and coding regions of the HXT1 and ERG9 genes for testing: ERG9 sgRNA: GAAAAGAGAGAGGAAG; HXT1 sgRNA: CCCATAATCAATTCCATCTG. Once vectors are completed, they will be mixed together for transformation.

2. Transformation of Yeast by Electroporation

1) Picked up better-grown mono-clones from a fresh plate and inoculated it with 5 mL YPD medium, grew with vigorously shaking 220 rpm at 30° C. for overnight. 2) Transferred to 50 mL YPD medium so that the initial OD₆₆₀ would be about 0.2, incubated with vigorously shaking 220 rpm at 30° C. to make OD₆₆₀ about 1.2. 3) After placing the yeast on ice for 30 min, centrifuged at 5000 g for 5 min at 4° C. to collect the cells. 4) Discarded the supernatant, washed the cells with pre-cooled sterile water twice, and then centrifuged. 5) Discarded the supernatant, washed the cells three times with pre-cooled 1 mol/L sorbitol solution. 6) Centrifuged to collect the cells, washed the cells three times with pre-cooled 200 μL 1 mol/L sorbitol solution. 7) Added 20 μL (about 5 μg) plasmids or DNA fragments to the cell suspension, gently mixed and incubated at ice for 10 min. 8) Transferred the mix into a pre-cooled cup, shocked 5 ms with 1500V. 9) Re-suspended the cells in the cup with 1 mL YPD medium and incubated at 30° C. with vortex for 1-2 hours. 10) Washed the recovered cells with sterile water, and finally re-suspended with 1 mL sterile water, took 100 μL on the corresponding plate. 11) Incubated at 30° C. thermostatic incubator for 3-5 days to select the transformers.

3. Extraction of Yeast Genome DNA

1) Took 5 ml overnight cultured medium, centrifuged to collect cells, after washed with 1 mL PBS twice, centrifuged to collect cells at maximum speed for 1 min; 2) Added 500 μL sorbitol buffer to re-suspend the cells and then added 50 U Lyticase, incubated at 37° C. for 4 h; 3) Centrifuged at 12000 rpm for 1 min to collect cells; 4) Added 500 μL yeast genomic DNA extraction buffer and re-suspended, added 50 μL 10% SDS, and placed immediately at 65° C. water bath for 30 min; 5) Added 200 μL 5M KAc (pH8.9), and incubated at ice for 1 h; 6) Centrifuged at 12000 rpm for 5 min at 4° C., and transferred supernatant to a new EP tube; 7) Added isopropyl alcohol of equal volume, centrifuged at 12000 rpm for 10 s; 8) discarded the supernatant and added 500 μL 75% ethanol to wash DNA, centrifuged at 12000 rpm for 1 min; 9) After precipitation, added 50 μL TE buffer to dissolve; 10) Took 3 μL DNA for electrophoresis test, the remaining was reserved in −20° C. refrigerator.

4. Detection of Inverted Events

PCR detection of transformed yeast cells using the following primers: HXT1pro-detF: TGCTGCGACATGATGATGGCTTT and ERG9cds-detR:TCGTGGAGAGTGACGACAAGT, respectively. The length of PCR product was expected to be 616 bp.

The invention replaces the yeast ERG9 gene promoter with the HXT1 promoter in vivo through the inversion editing event of the chromosome fragment between the target sites, which forms a new ERG9 gene regulated by glucose concentration. This example shows that the method of the present invention can be used to create new genes in fungal organisms.

### Example 28: Knock-Up Expression of EPO Gene in 293T Cell Line

EPO (erythropoietin), is an important cytokine in human, PSMC2 (proteasome 26S subunit ATPase 2) is a regulated subunit of 26S protease complex, ubiquitously expressed in cells. By designing a dual-target editing vector to identify and screen new EPO gene which would driven by PSMC2 promoter in 293T cell lines.

1. Target Design and Editing Vector Construction of CRISPR/Cas9

Using target design online tools of CRISPR (http://crispr.mit.edu/), sequence of 20 bp sgRNA oligos was designed in the 5′UTR region of the human EPO gene and PSMC2 gene, respectively. Oligos were synthesized by BGI Company (Qingdao, China. Diluted the synthetic sgRNA oligo to 100 μmol/L with deionized water. Reaction system (10 μL): for word oligo 1 μl, reverse oligo 1 μl, deionized water 8 μL; annealing program used for PCR: incubated 30 min at 37° C., incubated 5 min at 95° C., then gradually cool down to 25° C. at 5° C./min; diluted the oligo 250 times after annealing. The pX459 plasmid was firstly linearized with BbsI restriction enzyme, and then the annealing product was added, ligated product was transformed into DH5a competent cells. Single clones were selected into the centrifugal tube, incubated with shaking at 37° C. 12 to 16 hours, and then divided into 1 mL for sequencing. After sequence confirmation, plasmids were extracted. Preparation of the plasmid pX459-EPO and pX459-PSMC2 for transfection.

2. Resuscitation of 293T cell: removed the frozen tube from liquid nitrogen or −80° C. refrigerator container, immersed directly into warm water bath at 37° C., and shook it at interval to melt it as soon as possible; removed the frozen tube from the water bath at 37° C., opened the lid in the ultra-clean table, and sucked out the cell suspension with the tips (3 ml of cell complete media has been pre-added in the centrifugal tube), flicked and mixed; centrifuged at 1000 rpm for 5 min; discarded the supernatant, re-suspended cells gently, added 10% FBS cell media, re-suspended cells gently, adjusted cell density, inoculated at petri dishes, and incubated at 37° C. Replaced the cell media once the next day.

3. Transferred steps: removed cell petri dish (60 mm) from the carbon dioxide incubator, sucked out the medium in the bottle at the ultra-clean workbench, added 2 ml 1×PBS solution, gently rotated the petri dish to clean the cells, discarded the 1×PBS solution; added trypsin 0.5 ml and incubated for 3-5 minutes; during the incubation, observed the digested cells under an inverted microscope, and if the cells become round and no longer connected to each other, immediately added 2 volume complete medium (containing serum) in the ultra-clean workbench, added 1 mL of complete medium, blew and kept the cell suspended; the cell suspension was sucked out and placed in a 15 ml centrifugal tube, centrifuged at 1000 rpm for 5 min; discarded the digestive fluid and tapped the bottom of the centrifugal tube to make the cells re-suspended; added 2.5 ml complete medium into two new 60 mm petri dishes, the original digestive dish also added 2.5 ml of complete medium, and marked it; dropped the cell suspension in the centrifugal tube into three petri dishes at 0.5 ml/dish, blew cells with tips several times, and incubated in a carbon dioxide incubator.

4. Trypsin digested the cells and counted in a 100 mm petri dish, making them 60%-70% denser on the day of transfection. Added plasmid DNA with a maximum capacity of 24.0 μg into cell petri dish with a bottom area of 100 mm, diluted with 1.5 mL serum-free medium, mixed and incubated at 5 min at room temperature.

5. Cell transfection: (1) Diluted 80 μl LIPOFECTAMINE 2000 reagent with a 1.5 ml serum-free medium, and mixed diluted DNA within 5 minutes. (2) Mixed diluted plasmid DNA with diluted LIPOFECTAMINE 2000, incubated at room temperature for 20 minutes. (3) The above mixture was then added evenly to the cells. (4) Kept warm for 6 hours at 37° C., 5% CO₂, 100% saturated humidity, and added 12 ml of fresh DMEM culture with 10% FBS to each petri dish. After 24 hours, replaced the old medium with a fresh DMEM medium containing 10% FBS and keep incubating.

6. After 48 hours of transfection, centrifuged to collect cells. DNA from 293T cells was extracted using Tiangen's TIAN amp Genomic DNA Kit. The primers were also designed for PCR amplification of the target region.

### Example 29: Creation of New Genes with Different Expression Patterns by Translocation of Gene Promoter or Coding Region Fragment

A dual-target combination was designed for cutting off the promoter region of OsUbi2 gene at chromosome 2, wherein target 1 was just before the OsUbi2 initiation codon and target 2 was at the upstream of the OsUbi2 promoter. Third target (Target 3) was designed to cut between the promoter and the initiation codon of OsPPO2 gene at chromosome 4. The sgRNA sequences designed for the three targets were as following:

Target 1: OsUbi2pro-7NGGsgRNA:
5′gaaataatcaccaaacagat3′
Target 2: OsUbi2pro-1960NGGsgRNA:
5′atggatatggtactatacta3′
Target 3: OsPPO2cds-6NGGsgRNA:
5′ttggggctcttggatagcta3′,

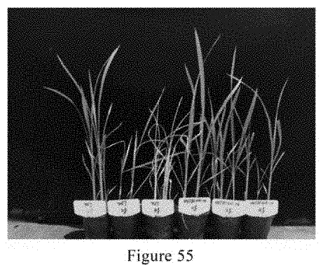

As shown in FIG. 54, new gene cassette, which is OsUbi2 promoter driving OsPPO2 gene, is created as a result of designed translocation. The translocation of OsUbi2 promoter resulted in the combination of the OsUbi2 promoter and the OsPPO2 coding region, which is a new gene or new gene expression cassette, ie. OsUbi2 promoter drives OsPPO2 expression. The calli or plantlets derived from the calli harboring such expected new gene may be obtained through PCR screening and genotyping.

The designed sgRNA sequences were ordered from GenScript Biotechnology Company (Nanjing, China). These sgRNAs were respectively assembled with SpCas9 forming RNP complexes, and three RNP complexes were mixed together in equal ratio. The mixture was subjected to biolistic transformation of rice calli (see WO2021088601A1 for specific experimental procedures).

The transformed calli were cultivated for 2 weeks and then sampled by using the following primer pair to test:

| | |
|---|---|
| OsUBi2pro-1648F: | 5′ggaatatgtttgctgtttgatccg3′ |
| OsPPO2-gDNA-236R: | 5′cagaactgaacccacggagag3′ |

PCR detection was preformed to detect whether new genes, which are OsUbi2 promoter driving OsPPO2, were generated. The translocation positive calli continued to be cultivated for 2 weeks, then followed by another round of PCR detection. After 3 rounds of detection, the positive calli were differentiated into seedlings, which were also sampled for PCR detection. The positive T0 seedlings were sequenced to identify the specific genotypes. A total of four different genotypes with OsUbi2 promoter driving OsPPO2 were obtained:

QY378-16: Ubi2pro + PPO2-CDS
5′CCCCCCTTTGGAATATGTTTGCTGTTTGATCCGTTGTTGTGTCCTTAA
TCTTGTGCTAGTTCTTACCCTATCTCCAAGAGCCCCAAATCAGATGCTCT
CTCCTGCCACCACCTTCTCCTCCTCCTCCTCCTCCTCGTCGCCGTCGCGC
GCCCACGCTCGCGCTCCCACCCGCTTCGCGGTCGCAGCATCCGCGCGCGC
CGCACGGTTCCGCCCCGCGCGCGCCATGGCCGCCTCCGACGACCCCCGCG
GCGGGAGGTCCGTCGCCGTCGTCGGCGCCGGCGTCAGT3′
QY378-18: Ubi2pro + PPO2-CDS
5′AATTGGAATATGTTTGCTGTTTGATCCGTTGTTGTGTCCTTAATCTTG
TGTTGTGTCCTTAATCCAAGAGCCCCAAATCAGATGCTCTCTCCTGCCAC
CACCTTCTCCTCCTCCTCCTCCTCCTCGTCGCCGTCGCGCGCCCACGCTC
GCGCTCCCACCCGCTTCGCGGTCGCAGCATCCGCGCGCGCCGCACGGTTC
CGCCCCGCGCGCGCCATGGCCGCCTCCGACGACCCCCGCGGCGGGAGGTC
CGTCGCCGTCGTCGGCGCCGGCGTCAGTGG3′
QY378-41: Ubi2pro + PPO2-CDS
5′ATCTGTGCTAGTTCTTaCCCTATCTCCAGAGCCCCAAATCAGATGCTC
TCTCCTGCCACCACCTTcTCCTCCTCCTCCTCCTCCTCGTCGCCGTCGCG
CGCCCACGCTCGCGCTCCCACCCGCTTCGCGGTCGCAGCATCCGCGCGCG
CCGCACGGTTCCGCCCCGCGCGCGCCATGGCCGCCTCCGACGACCCCCGC
GGCGGGAGGTCCGTCGCCGTCGTCGGCGCCGGCGTCAGGTGG3′
QY378-374: Ubi2pro + PPO2-CDS
5′GGTGGTCTATCTTGTGTTGTGTCCTTATCCAGAGCCCCAAATCAGATG
CTCTCTCCTGCCACCACCTTCTCCTCCTCCTCCTCCTCCTCGTCGCCGTC
GCGCGCCCACGCTCGCGCTCCCACCCGCTTCGCGGTCGCAGCATCCGCGC
GCGCCGCACGGTTCCGCCCCGCGCGCGCCATGGCCGCCTCCGACGACCCC
CGCGGCGGGAGGTCCGTCGCCGTCGTCGGCGCCGGCGTCAGGTG3′

The T1 generation seedlings were harvested from T0 plants, then tested using PCR. The results confirmed that the above genotypes could be inherited stably. The T1 generation of QY378-16 were selected and treated with compound A by foliar spray. As shown in FIG. 55, it showed significantly improved resistance to PPO-inhibiting herbicide Compound A. The wild-type rice was killed at the rate of 2 g a.i./mu, while the T1 generation of QY378-16 bearing Ubi2pro+PPO2-CDS genotype could survive a rate of 4 g a.i./mu, showing that the new PPO2 gene improved plant tolerance to Compound A.

By referring to this technical route, different target combinations were designed for OsUBi2, OsPPO2 and OsPPO1 using SpCas9 protein as the editing agent:

| | |
|---|---|
| 1. OsUbi2pro- | 5′atggatatggtactatacta3′ |
| 1960NGGsgRNA: | |
| 2. OsUbi2pro-7NGGsgRNA: | 5′atctttgtgaagacattgac3′ |
| 3. OsPPO2cds-6NGGsgRNA: | 5′ttggggctcttggatagcta3′ |
| 4. OsPPO2cds-14NGGsgRNA: | 5′gcaggagagagcatctgatt3′ |
| 5. OsPPO1cds-4NGGsgRNA: | 5′ccatgtccgtcgctgacgag3′ |

The combination of sgRNA 1+2+3 and sgRNA 1+2+4 with Cas9 protein was subjected to RNP transformation, new heritable genes with Ubi2pro+PPO2-CDS were identified after PCR screen selection. Similarly, the combination of sgRNA 1+2+5 with Cas9 protein was also subjected to RNP transformation, new heritable genes with Ubi2 promoter driving PPO1-CDS were also obtained.

Using MAD7 Protein as the Editing Agent:

| | |
|---|---|
| 1. OsUbi2pro- | 5′gttggaggtcaaaataacagg3′ |
| 1896MAD7crRNA: | |
| 2. OsUbi2pro- | 5′tgaagacattgaccggcaaga3′ |
| 14MAD7crRNA: | |
| 3. OsUbi2pro- | 5′gtgattatttcttgcagatgc3′ |
| 17MAD7crRNA: | |
| 4. OsPPO2cds- | 5′gggctcttggatagctatgga3′ |
| 9MAD7crRNA: | |
| 5. OsPPO1cds- | 5′ccattccggtgggccattccg3′ |
| 125MAD7crRNA: | |

The combination of crRNA 1+2+4 and crRNA 1+3+4 with MAD7 protein was subjected to RNP transformation, new heritable genes with Ubi2pro+PPO2-CDS were identified after PCR screen selection. Similarly, the combination of crRNA 1+2+5 and crRNA 1+3+5 added with MAD7 protein was subjected to RNP transformation, new heritable genes with Ubi2 promoter driving PPO1-CDS were also obtained.

In these examples, a new gene with different expression pattern was generated by inserting a translocated promoter upstream of the coding region of another gene. Likewisely, following the same technique idea, a new gene with different expression pattern could also be generated by inserting a translocated gene coding region into the downstream region of another promoter, which is covered by the technical solution scope of the present application.

All publications and patent applications mentioned in the description are incorporated herein by reference, as if each publication or patent application is individually and specifically incorporated herein by reference.

Although the foregoing invention has been described in more detail by way of examples and embodiments for clear understanding, it is obvious that certain changes and modifications can be implemented within the scope of the appended claims, such changes and modifications are all within the scope of the present invention.

1. A method for creating a new gene in an organism, characterized by comprising the following steps:
simultaneously generating DNA breaks at two or more different specific sites in the organism's genome, wherein the specific sites are genomic sites capable of separating different genetic elements or different protein domains, and the DNA breaks are ligated to each other by a non-homologous end joining (NHEJ) or homologous repair, generating a new combination of the different genetic elements or different protein domains different from the original genomic sequence, thereby creating the new gene; or
a method for in vivo creation of new genes that can be stably inherited in an organism, characterized by comprising the following steps:
(1) simultaneously generating double-stranded DNA breaks at two or more different specific sites in the organism's genome, wherein the specific sites are capable of separating different gene elements or different protein domains, and the DNA breaks are then ligated to each other by a non-homologous end joining (NHEJ) or homologous repair, generating a new combination or assemble of the different gene elements or different protein domains derived from the original genomic sequence, thereby the new gene is generated;
optionally, it also includes (2) designing primer pairs that can specifically detect the above-mentioned new combination or assemble, then cells or tissues containing the new genes can be screened out by PCR test, and the characteristic sequences of new combinations of gene elements can be determined by sequencing; and
(3) cultivating the above-screened cells or tissues to obtain T0 generation organisms, and perform PCR tests and sequencing on the organisms for two consecutive generations including the T0 generation and its bred T1 or at least three consecutive generations to select the organisms containing the above-mentioned characteristic sequence of new combination of gene elements, namely, a new gene that can be stably inherited has been created in the organism;
optionally, it also includes (4) testing the biological traits or phenotypes related to the function of the new gene, to determine the genotype that can bring beneficial traits to the organism, and to obtain a new functional gene that can be stably inherited.

2. The method according to claim 1, wherein in the step (1), DNA breaks are simultaneously generated at two different specific sites in the genome of the organism, wherein one site is the genomic locus between the promoter region and the coding region of a gene, meanwhile, the other site is between the promoter region and the coding region of another gene with different expression patterns, resulting in a new combination of the promoter of one gene and the coding region of the other gene that has a different expression pattern; or a combination of the strong promoter and the gene of interest is eventually produced.

3. The method according to claim 1, wherein in the step (1), DNA breaks are simultaneously generated at three different specific sites in the genome of the organism, the three specific sites include two genomic sites whose combination capable of cutting off the promoter region of a highly expressed gene and the third genomic site between the coding region and the promoter region of the gene of interest that has a different expression pattern; or a genomic site between the promoter region and the coding region of a highly expressed gene and another two genomic sites whose combination capable of cutting off the coding region fragment of the gene of interest that has a different expression pattern; then through gene editing at the above-mentioned sites, translocation editing events can be generated, in which the strong promoter fragment that is inserted upstream of the coding region of the gene of interest, or the coding region fragment of the gene of interest is inserted the downstream of the promoter of another highly expressed gene, finally, the combination of the promoter of one gene and the coding region of the other gene of interest with different expression patterns is generated.

4. The method according to claim 1, characterized in that said two or more different specific sites locate on the same chromosome or on different chromosomes; optionally, said two or more different specific sites may be specific sites on at least two different genes, or may be at least two different specific sites on the same gene; and said at least two different genes may have the same or different transcription directions.

5. The method according to claim 1, characterized in that said gene elements are selected from the group consisting of a promoter, a 5′ untranslated region, a coding region or non-coding RNA region, a 3′ untranslated region, a terminator of the gene, or any combination thereof.

6. The method according to claim 1, characterized in that the combination of different gene elements is a combination of the promoter of one of the two genes with different expression patterns and the coding region or the non-coding RNA region of the other gene, or the combination of different gene elements is a combination of the region from the promoter to 5′UTR of one of the two genes with different expression patterns and the CDS or non-coding RNA region of the other gene, or the combination of different gene elements is a combination of adjacent gene elements of the same gene.

7. The method according to any claim 1, characterized in that the protein domain is a DNA fragment corresponding to a specific functional domain of a protein including a nuclear localization signal, a chloroplast leading peptide, a mitochondrial leading peptide, a phosphorylation site, a methylation site, a transmembrane domain, a DNA binding domain, a transcription activation domain, a receptor activation domain, or an enzyme catalytic center.

8. The method according to claim 1, characterized in that the combination of different protein domains is a combination of the localization signal region of one of two proteins with different subcellular localizations and the mature protein coding region of the other gene, or a combination of two protein domains with different biological functions, or a combination of adjacent protein domains of the same gene; wherein the different subcellular locations are selected from the group consisting of nuclear location, cytoplasmic location, cell membrane location, chloroplast location, mitochondrial location, and endoplasmic reticulum membrane location; or the different biological functions are selected from the group consisting of recognition of specific DNA or RNA conserved sequence, activation of gene expression, binding to a protein ligand, binding to small molecular signal, binding to an ion, specific enzymatic reaction, and any combination thereof.

9. The method according to claim 1, characterized in that the combination of gene elements and protein domains are a combination of protein domains and adjacent promoters, 5′UTR, 3′UTR or terminators of the same gene.

10. The method according to claim 1, characterized in that the organism is a non-human animal, a plant or a fungus.

11. The method according to claim 1, characterized in that the combination of different gene elements is selected from any of the following:
(1) one element is a plant endogenous strong promoter or the region from a strong promoter to 5′UTR, and the other is the HPPD, EPSPS, PPO, ALS, ACCase, GS, PDS, DHPS, DXPS, HST, SPS, cellulose synthesis, VLCFAS, fatty acid thioesterase, serine threonine protein phosphatase or lycopene cyclase gene coding region of the same plant;
(2) one element is an endogenous strong promoter or the region from a strong promoter to 5′UTR of the organism, and the other is a gene coding region of any one of the P450 family in the same organism;
(3) one element is a rice or maize endogenous strong promoter or the region from a strong promoter to 5′UTR, and the other is a gene coding region of OsCYP81A gene or ZmCYP81A9 gene in the same organism;
(4) one element is a maize endogenous strong promoter or the region from a strong promoter to 5′UTR, and the other is the coding region of maize gene ZMM28 (Zm00001d022088), ZmKNR6 or ZmBAM1d;
(5) one element is a rice endogenous strong promoter or the region from a strong promoter to 5′UTR, and the other is the coding region of rice gene COLD1 or OsCPK24;
(6) one element is an endogenous strong promoter or the region from a strong promoter to 5′UTR of the organism, and the other is a gene coding region of any one of the ATP-binding cassette (ABC) transporter family in the same organism;
(7) one element is a plant endogenous strong promoter or the region from a strong promoter to 5′UTR of the plant, and the other is a gene coding region of any one of the NAC transcription factor family (for example, OsNAC045, OsNAC67, ZmSNAC1, OsNAC006, OsNAC42, OsSNAC1 or OsSNAC2) in the same plant;
(8) one element is a plant endogenous strong promoter or the region from a strong promoter to 5′UTR, and the other is the gene coding region of any one of MYB, MADS, DREB and bZIP transcription factor family in the same plant;
(9) one element is the promoter of any one of overexpression or tissue-specific expression rice genes listed in Table A, and the other is the protein coding region or the non-coding RNA region of another gene that is different from the selected promoter corresponding to the rice gene;
(10) one element is a protein coding region or non-coding RNA region selected from any one of the biological functional genes listed in Table B to K, and the other is the promoter region of another gene that is different from the selected functional gene of the biological genome corresponding to the selected gene;
(11) one element is an endogenous strong promoter or the region from a strong promoter to 5′UTR of the organism, and the other is a gene coding region of any one of the GST (glutathione-s-transferases) family in the same organism;
(12) one element is a wheat or maize endogenous strong promoter or the region from a strong promoter to 5′UTR of the organism, and the other is a gene coding region of wheat GST Cla47 (AY064480.1) gene, wheat GST 19E50 (AY064481.1), wheat GST28E45 (AY479764.1), maize ZmGSTIV, maize ZmGST6, maize ZmGST31, maize GSTI, maize GSTIII, maize GSTIV, maize GST5 or maize GST7 gene in the same organism;
(13) one element is a rice endogenous strong promoter or the region from a strong promoter to 5′UTR, and the other is the coding region of any one of gene protein in rice GIF1 (Os04g0413500), NOG1 (Os01g075220), LAIR (Os02g0154100), OSA1 (Os03g0689300), OsNRT1.1A (Os08g0155400), OsNRT2.3B (Os01g0704100), OsRac1 (Os01g0229400), OsNRT2.1 (Os02g0112100), OsGIF1 (Os03g0733600), OsNAC9 (Os03g0815100), CPB1/D11/GNS4 (Os04g0469800), miR1432 (Os04g0436100), OsNLP4 (Os09g0549450), RAG2 (Os07g0214300), LRK1 (Os02g0154200), OsNHX1 (Os07t0666900), GW6 (Os06g0623700), WG7 (Os07g0669800), D11/OsBZR1 (Os04g0469800, Os07g0580500), OsAAP6 (Os07g0134000), OsLSK1 (Os01g0669100), IPA1 (Os08g0509600), SMG11 (Os01g0197100), CYP72A31 (Os01g0602200), SNAC1 (Os03g0815100), ZBED (Os01g0547200), OsSta2 (Os02g0655200), OsASR5 (Os11g0167800), OsCPK4 (Os02g03410), OsDjA9 (Os06g0116800), EUI (Os05g0482400), JMJ705 (Os01g67970), WRKY45 (Os05t0322900), OsRSR1 (Os05g0121600), OsRLCK5 (Os01g0114100), APIP4 (Os01g0124200), OsPAL6 (Os04t0518400), OsPAL8 (Os11g0708900), TPS46 (Os08t0168000), OsERF3 (Os01g58420) and OsYSL15 (Os02g0650300);
(14) one element is a fish endogenous strong promoter, and the other is a gene coding region of GH1 (growth hormone 1) in the selected fish; or the combination of different protein domains is selected from any of the following:
(a) one element is a wheat endogenous protein chloroplast localization signal domain, and the other is a wheat mature protein coding region of cytoplasmic localization phosphoglucose isomerase (PGIc);
(b) one element is a rice protein chloroplast localization signal domain (CTP), and the other is the mature protein coding region of OsGLO3, OsOXO3 or OsCATC.

12. A new gene created by the method according to claim 11, characterized in that the new genes formed by any one of the combinations of the different gene elements (1)-(14) respectively have the following characters:
(1) the level of the new gene expression is up-regulated relative to the plant endogenous wild-type HPPD, EPSPS, PPO, ALS, ACCase, GS, PDS, DHPS, DXPS, HST, SPS, cellulose synthesis, VLCFAS, fatty acid thioesterase, serine threonine protein phosphatase or lycopene cyclase gene;
(2) the level of the new gene expression is up-regulated relative to the corresponding endogenous wild-type P450 gene of the organism;
(3) the level of the new gene expression is up-regulated relative to the rice endogenous OsCYP81A6 gene or maize endogenous ZmCYP81A9 gene, respectively;
(4) the level of the new gene expression is up-regulated relative to the plant endogenous wild-type ZMM28 gene, ZmKNR6 gene or ZmBAM1d gene, respectively;
(5) the level of the new gene expression is up-regulated relative to the rice endogenous wild-type COLD1 or OsCPK24 gene, respectively;
(6) the level of the new gene expression is up-regulated relative to the corresponding endogenous wild-type ATP-binding cassette (ABC) transporter gene of the organism;
(7) the level of the new gene expression is up-regulated relative to the corresponding plant endogenous wild-type NAC transcription factor family gene;
(8) the level of the new gene expression is up-regulated relative to the corresponding plant endogenous wild-type MYB transcription factor gene, MADS transcription factor family gene, DREB transcription factor family gene coding region or bZIP transcription factor family gene, respectively;
(9) the expression pattern of the new gene is changed relative to the selected protein coding region or the non-coding RNA region of the rice endogenous gene;
(10) the expression pattern of the new gene is changed relative to the selected functional gene;
(11) the level of the new gene expression is up-regulated relative to the corresponding endogenous GST (glutathione-s-transferases) family gene of the organism;
(12) the level of the new gene expression is up-regulated relative to the endogenous wheat GST Cla47 (AY064480.1) gene, wheat GST 19E50 (AY064481.1), wheat GST28E45 (AY479764.1), maize ZmGSTIV, maize ZmGST6, maize ZmGST31, maize GSTI, maize GSTIII, maize GSTIV, maize GST5 or maize GST7 gene, respectively;
(13) the level of the new gene expression is up-regulated relative to the corresponding endogenous gene;
(14) the new gene is a fish endogenous high expression GH1 gene; or the new genes formed by any one of the combinations of the different protein domains (a)-(b) respectively have the following characters:
(a) the new gene locates the phosphoglucose isomerase gene relative to the coding cytoplasm and its mature protein is located in the chloroplast.
(b) the mature protein of the new gene is located in chloroplast different from OsGLO3, OsOXO3 or OsCATC.

13.-17. (canceled)

18. A chloroplast localized protein OsCACT or OsGLO3, the nucleotide encoding the protein has a sequence selected from the group consisting of:
(1) the nucleic acid sequence as shown in SEQ ID NO: 28 or a portion thereof or a complementary sequence thereof, a sequence having an identity of at least 75%, at least 80%, at least 85%, at least 90%, at least 95%, at least 98% or at least 99% thereto, or a nucleic acid sequence capable of hybridizing to one of the foregoing sequences under a stringent condition; and
(2) the nucleic acid sequence as shown in SEQ ID NO: 29 or a portion thereof or a complementary sequence thereof, a sequence having an identity of at least 75%, at least 80%, at least 85%, at least 90%, at least 95%, at least 98% or at least 99% thereto, or a nucleic acid sequence capable of hybridizing to one of the foregoing sequences under a stringent condition.

19.-20. (canceled)

21. An editing method for regulating the gene expression level of a target endogenous gene in an organism, which is independent of an exogenous DNA donor fragment, which comprises the following steps:
simultaneously generating DNA breaks separately at selected sites between the promoter and the coding region of each of the target endogenous gene and an optional endogenous inducible or tissue-specific expression gene with a desired expression pattern; ligating the DNA breaks to each other by means of non-homologous end joining (NHEJ) or homologous repair, thereby generating an in vivo fusion of the coding region of the target endogenous gene and the optional inducible or tissue-specific expression promoter to form a new gene with expected expression patterns, the target endogenous gene and the optional endogenous inducible or tissue-specific expression gene with a desired expression pattern are located on the same chromosome or on different chromosomes; optionally, the target endogenous gene is yeast ERG9 gene, the endogenous inducible expression gene is HXT1 gene, and the inducible expression promoter is HXT1 in response to glucose concentration.

22. A yeast endogenous inducible ERG9 gene obtainable by the editing method according to claim 21.

23. (canceled)

24. A highly-expressing rice endogenous HPPD gene, rice endogenous PPO2 gene, maize endogenous PPO2 gene, wheat endogenous PPO2 gene, or oilseed rape endogenous PPO2 gene, which has a sequence selected from the group consisting of:
(1) the nucleic acid sequence as shown in SEQ ID NO: 27 or a portion thereof or a complementary sequence thereof, a sequence having an identity of at least 75%, at least 80%, at least 85%, at least 90%, at least 95%, at least 98% or at least 99% thereto or a nucleic acid sequence capable of hybridizing to one of the foregoing sequences under a stringent condition;
(2) the nucleic acid sequence as shown in SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO:41, SEQ ID NO: 42, SEQ ID NO: 43, SEQ ID NO: 44, SEQ ID NO: 45, SEQ ID NO: 46 or SEQ ID NO: 47 or a portion thereof or a complementary sequence thereof, a sequence having an identity of at least 75%, at least 80%, at least 85%, at least 90%, at least 95%, at least 98% or at least 99% thereto, or a nucleic acid sequence capable of hybridizing to one of the foregoing sequences under a stringent condition;
(3) the nucleic acid sequence as shown in SEQ ID NO: 48 or SEQ ID NO: 49 or a portion thereof or a complementary sequence thereof, a sequence having an identity of at least 75%, at least 80%, at least 85%, at least 90%, at least 95%, at least 98% or at least 99% thereto, or a nucleic acid sequence capable of hybridizing to one of the foregoing sequences under a stringent condition;
(4) the nucleic acid sequence as shown in SEQ ID NO: 50, SEQ ID NO: 5, SEQ ID NO: 52, SEQ ID NO: 53, SEQ ID NO: 54, SEQ ID NO: 55 or SEQ ID NO: 56 or a portion thereof or a complementary sequence thereof, a sequence having an identity of at least 75%, at least 80%, at least 85%, at least 90%, at least 95%, at least 98% or at least 99% thereto, or a nucleic acid sequence capable of hybridizing to one of the foregoing sequences under a stringent condition; and
(5) the nucleic acid sequence as shown in SEQ ID NO: 57, SEQ ID NO: 58, SEQ ID NO: 59, SEQ ID NO: 60 or SEQ ID NO: 61 or a portion thereof or a complementary sequence thereof, a sequence having an identity of at least 75%, at least 80%, at least 85%, at least 90%, at least 95%, at least 98% or at least 99% thereto, or a nucleic acid sequence capable of hybridizing to one of the foregoing sequences under a stringent condition.

25.-29. (canceled)

30. A plant or a progeny derived therefrom regenerated from the plant cell which comprises the gene (1), (3) or (12) set forth in claim 12.

31. A method for producing a plant with an increased resistance or tolerance to an herbicide, which comprises regenerating the plant cell which comprises the gene (1), (3) or (12) set forth in claim 12 into a plant or a progeny derived therefrom.

32.-33. (canceled)

34. A method for controlling a weed in a cultivation site of a plant, wherein the plant is selected from the group consisting of a plant prepared by the method according to claim 31, wherein the method comprises applying to the cultivation site one or more corresponding inhibitory herbicides in an amount for effectively controlling the weed; wherein the herbicide comprises one or a combination of two or more of inhibition of HPPD, inhibition of EPSPS, inhibition of PPO, inhibition of ALS, inhibition of ACCase, inhibition of GS, inhibition of PDS, inhibition of DHPS, inhibition of DXPS, inhibition of HST, inhibition of SPS, inhibition of cellulose synthesis, inhibition of VLCFAS, inhibition of fatty acid thioesterase, inhibition of serine threonine protein phosphatase or inhibition of lycopene cyclase herbicides.

35. (canceled)

36. An editing method for knocking up the expression of an endogenous WAK gene or CNGC gene in a plant, characterized in that it comprises fusing the coding region of the WAK gene or CNGC gene with a strong endogenous promoter of a plant in vivo to form a new highly-expressing plant endogenous WAK gene or CNGC gene, respectively; or it comprises the following steps: simultaneously generating DNA breaks respectively in selected specific sites between the promoter and the coding region of each of the WAK gene or CNGC gene and an optional endogenous highly-expressing gene, ligating the DNA breaks to each other through an intracellular repair pathway, generating in vivo a fusion of the coding region of the WAK gene or CNGC gene and the optional strong endogenous promoter to form a new highly-expressing WAK gene or CNGC gene.

37. A highly-expressing plant endogenous WAK gene or CNGC gene obtainable by the editing method according to claim 36, wherein the highly-expressing rice WAK gene or CNGC gene has a sequence selected from the group consisting of:
(1) the nucleic acid sequence as shown in SEQ ID NO: 62, SEQ ID NO: 63, SEQ ID NO: 64, SEQ ID NO: 65, SEQ ID NO: 66, SEQ ID NO: 67 or SEQ ID NO: 68 or a portion thereof or a complementary sequence thereof, a sequence having an identity of at least 75%, at least 80%, at least 85%, at least 90%, at least 95%, at least 98% or at least 99% thereto, or a nucleic acid sequence capable of hybridizing to one of the foregoing sequences under a stringent condition; and
(2) the nucleic acid sequence as shown in SEQ ID NO: 69, SEQ ID NO: 70, SEQ ID NO: 71 or SEQ ID NO: 72 or a portion thereof or a complementary sequence thereof, a sequence having an identity of at least 75%, at least 80%, at least 85%, at least 90%, at least 95%, at least 98% or at least 99% thereto, or a nucleic acid sequence capable of hybridizing to one of the foregoing sequences under a stringent condition.

38.-40. (canceled)

41. A rice resistant to rice blast, which comprises one or a combination of two or more of the highly-expressing rice WAK or CNGC gene according to claim 37.

42. An editing method for knocking up the expression of an endogenous GH1 gene in a fish, IGF2 (Insulin-like growth factor 2) gene in a pig, or IGF1 (Insulin-like growth factor 1) gene in a chicken embryo fibroblast, characterized in that it comprises fusing the coding region of the GH1, IGF2 or IGF1 gene with a strong endogenous promoter of a fish, pig or chicken in vivo to form a new highly-expressing fish endogenous GH1, pig endogenous IGF2 or chicken endogenous IGF1 gene, respectively; or it comprises the following steps: simultaneously generating DNA breaks respectively in selected specific sites between the promoter and the coding region of each of the GH1, IGF2 or IGF1 gene and an optional endogenous highly-expressing gene, ligating the DNA breaks to each other through an intracellular repair pathway, generating in vivo a fusion of the coding region of the GH1, IGF2 or IGF1 gene and the optional strong endogenous promoter to form a new highly-expressing GH1, IGF2 or IGF1 gene, respectively; wherein the strong promoter of a fish is the corresponding fish Col1A1a (Collagen type I alpha 1a) gene promoter, RPS15A (ribosomal protein S15a) gene promoter, Actin promoter or DDX5 [DEAD (Asp-Glu-Ala-Asp) box helicase 5] gene promoter, the strong promoter of a pig is one of the pig TNNI2 and TNNT3 gene promoter, the strong promoter of a chicken is chicken MYBPC1 (myosin binding protein C) gene promoter.

43.-44. (canceled)

45. A highly-expressing fish endogenous GH1 gene, a highly-expressing pig endogenous IGF2 gene or a highly-expressing chicken endogenous IGF1 gene obtainable by the editing method according to claim 42.

46. (canceled)

47. An editing method for knocking up the expression of an endogenous EPO (Erythropoietin) or p53 gene in an animal cell, characterized in that it comprises fusing the coding region of the EPO or p53 gene with a strong endogenous promoter of an animal in vivo to form a new highly-expressing endogenous EPO or p53 gene; or it comprises the following steps: simultaneously generating DNA breaks respectively in selected specific sites between the promoter and the coding region of each of the EPO or p53 gene and an optional endogenous highly-expressing gene, ligating the DNA breaks to each other through an intracellular repair pathway, generating in vivo a fusion of the coding region of the EPO or p53 gene and the optional strong endogenous promoter to form a new highly-expressing EPO or p53 gene.

48. A highly-expressing animal endogenous EPO or p53 gene obtainable by the editing method according to claim 47.

49. (canceled)

50. The method according to claim 1, characterized in that said DNA breaks are achieved by delivering a nuclease with targeting property into a cell of the organism to contact with the specific sites of the genomic DNA; wherein said nuclease with targeting property is selected from the group consisting of Meganuclease, Zinc finger nuclease, TALEN, and CRISPR/Cas system.

51. The method according to claim 50, characterized in that the nucleases with targeting property are delivered into the cell by: 1) a PEG-mediated cell transfection method; 2) a liposome-mediated cell transfection method; 3) an electric shock transformation method; 4) a microinjection; 5) a gene gun bombardment; 6) an Agrobacterium-mediated transformation method; 7) viral vector-mediated transformation method; or 8) nanomagnetic bead mediated transformation method.

52.-57. (canceled)

58. A composition, which comprises:
(a) a promoter of one of two genes with different expression patterns and a coding region or non-coding RNA region of the other gene;
(b) a promoter to a 5′ untranslated region of one of two genes with different expression patterns and a coding region or non-coding RNA region of the other gene;
(c) a localization signal region of one of the two protein coding genes with different subcellular localizations and a mature protein coding region of the other gene;
(d) DNA regions coding two different functional domains that come from two different functional protein-coding genes; wherein, the combination of gene elements said is not naturally exist, but a joined chromosome segment as designed and stable inheritance;
which is fused in vivo; and the different expression patterns are different levels of gene expression, different tissue-specific of gene expression, or different developmental stage-specificities of gene expression; or the different subcellular locations are selected from the group consisting of nuclear location, cytoplasmic location, cell membrane location, chloroplast location, a mitochondrial location, an endoplasmic reticulum membrane location, and any combination thereof; or the different biological functions are selected from the group consisting of recognition of specific DNA or RNA conserved sequence, activation of gene expression, binding to protein ligand, binding to small molecular signal, binding to an ion, specific enzymatic reaction, and any combination thereof.


SEQUENCE LISTING (72 sequences)

SEQ ID NO: 1 - DNA, 4439 bp, Arabidopsis thaliana
  Sequence of Arabidopsis thaliana PPO gene (Genbank number AT4G01690)
gatattttgatgtcgtatgtatatcaatgttgttagctgtaaactgtccaatgaaaatccatttaatatttggacaaattattattagggaaattaatgtaatgtggagtattttatatgaaatgtgtattaatgacgacgaccttacttgatcccggaagtgtgtattaatttaatcgcggtgcatcgctgtccacacggttgggttcatgcgtacgtggtatttgctggcaagtggcagtgaagtttcttgtttgatactgtatgttccattcatgtctttccctagatgcatgtttcttctatttttatatattaatatttttttgtttttcttattatgttttctttccctttgtgaggagaataattgcgatacgattatttgttttaacagaaaaaagtgaagcgaaaaagatgattcgaagtttgaagtacgattaaaatttttctttttccggatccgagtttgatcttctttagaaacaaaaattatatttgtttagaaagtctgtcagttgggttttggcccaattgatttacctggtagtcagaaatgtaagtgacctgactacccttgacattagtcagaaaacattctaaactcgagataaacactgtggtagttctttagaaagaagtcgactctaaagtactttggcgtaaaacaaaaaaatttcccttgaaattatactttatttgtttgacataaatgtttcaaatataatcttcttttaatcaatgtagttaaatttttttgatagaagcacgtcagaatttcacactagtaatgtataagtggtaatcatcattgcctaaaacaatgattaattaaaattttagaattcgtcgtcgacaaagaatttttcttatgatgaaggttttgtgcataattatgaacggatccatttatttcactctaactaggaatagatgtttgatgggatacaagtgaacacgacacaaagtaacaaaaattcatttaacgtcacaaattattctcaatcgaatttcgaagtatcaagaaaaagggcaattaaaaacgtaatctcattatgtctttgaaatctaaattaaaagttgagtttattttaattattaatttcctttttgatgatgtaggggatgacaattgacaaccaatttgatttgaaagattaacataaatcattttgagcatacatatattgcttaaatcacacccgtttatctctaaaaacctccataatttatatcaggaaaataaatctagttattgatatcgccatcaaatatttttttcttaaattgtttttttttgttgttgttgaaaatttaattagaaagataagtggacaacgacgcgagacaaagacacaatctttagggccttgagtaaattagggacagcataaacacatgcactagtcctgtctttatttaattatgtggtgtttactctaatgacgtcgctttgattaaccaagtggtcataattaatatatcgatcgaattatataattatcataaatttgaataagcatgttgccttttattaaagaggtttaataaagtttggtaataatggactttgacttcaaactcgattctcatgtaattaattaatatttacatcaaaatttggtcactaatattaccaaattaatatactaaaatgttaattcgcaaataaaacactaattccaaataaagggtcattatgataaacacgtattgaacttgataaagcaaagcaaaaataatgggtttcaaggtttggttatatatgacaaaaaaaaaaaaaggtttggttatatatctattgggcctataaccatgttataacaaatttgggcctaactaaaataataaaataaacgtaatggtcctttttatatttgggtcaaacccaactctaaacccaaaccaaagaaaaagtatacggtacggtacacagacttatggtgtgtgtgattgcaggtgaatatttctcgtcgtcttctcctttcttctgaagaagattacccaatctgaaaaaaaccaagaagctgacaaaattccgaattctctgcgatttccatggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtaagttcttgaaacaatctgattcgttaatactgagaacaatgtatttgtgaattgtttgatttgtggatttagccttctctgtttatggttagagtaggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccagtgtgtattcttacgaccttttagatcaatatgatttttagggctttatgttggtagaatctgctcatgttcttagagtttggcaaaggtgacagggtcgtgaagaatctgtggaggagtttgtacggcgtaacctcggtgatgaggtttttgagcgcctgattgaaccgttttgttcaggtagagttatagataaaacttcatgtgaaccatttattcatttagatgtcagcgaatctgattatctagcagttttcatttttgttctagtgcacccttaatgctttagattttatgcttttaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgtgagtaacatcaaacttttctgttgcttgggtcttttgtcccttcatatatatatttgtaaaacaactcaacctgcttcttcaggcgcctgccaaaaccacagggccaaacagttggttctttcaggaagggacttcgaatgttgccagaagcaatatctgcaaggtatccagtacttgtctctctctattgttggaagggaaaagggaaaaactttcaaatgttttctgaaatcttttgcactttgacagattaggtagcaaagttaagttgtcttggaagctctcaggtatcactaagctggagagcggaggatacaacttaacatatgagactccagatggtttagtttccgtgcagagcaaaagtgttgtaatgacggtgccatctcatgttgcaagtggtctcttgcgccctctttctgtaagtttctcattttgcgaactaggaatttgctcaaaattcatagattcgacaaatatgggttcttaaatcgtgtgctgcaactgttatcttctcaggaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattaggtatatatcttgtagttataatcgtcaattatgtcaaaatgttcatagaatcttcatgctgttgctcgtatttcttcaggaactatctacagctcctcactctttccaaatcgcgcaccgcccggaagaattttgctgttgaactacattggcgggtctacaaacaccggaattctgtccaaggtaaaaaacagcaaacacttgtaacacatctttattcaaccaagtaaacctaagaactgatagttttcttctctctctctcttttgttttgattccgcagtctgaaggtgagttagtggaagcagttgacagagatttgaggaaaatgctaattaagcctaattcgaccgatccacttaaattaggagttagggtatggcctcaagccattcctcagtttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 2 - DNA, 3452 bp, Arabidopsis thaliana
  Sequence of Arabidopsis thaliana Ubiquitin10 gene (Genbank number AT4G05320)
aaacaagactaccaatatgatgaactgatgatttttttttttttttttttggctcaacaacaaactttccattcacccatttatggatacacagtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatctttgttaagactctcaccggaaagacaatcaccctcgaggtggaaagctccgacaccatcgacaacgttaaggccaagatccaggataaggagggcattcctccggatcagcagaggcttattttcgccggcaagcagctagaggatggccgtacgttggctgattacaatatccagaaggaatccaccctccacttggtcctcaggctccgtggtggtatgcagattttcgttaaaaccctaacgggaaagacgattactcttgaggtggagagttctgacaccatcgacaacgtcaaggccaagatccaagacaaagagggtattcctccggaccagcagaggctgatcttcgccggaaagcagttggaggatggcagaactcttgctgactacaatatccagaaggagtccacccttcatcttgttctcaggctccgtggtggtatgcagattttcgttaagacgttgactgggaaaactatcactttggaggtggagagttctgacaccattgataacgtgaaagccaagatccaagacaaagagggtattcctccggaccagcagagattgatcttcgccggaaaacaacttgaagatggcagaactttggccgactacaacattcagaaggagtccacactccacttggtcttgcgtctgcgtggaggtatgcagatcttcgtgaagactctcaccggaaagaccatcactttggaggtggagagttctgacaccattgataacgtgaaagccaagatccaggacaaagagggtatcccaccggaccagcagagattgatcttcgccggaaagcaacttgaagatggaagaactttggctgactacaacattcagaaggagtccacacttcacttggtcttgcgtctgcgtggaggtatgcagatcttcgtgaagactctcaccggaaagactatcactttggaggtagagagctctgacaccattgacaacgtgaaggccaagatccaggataaggaaggaatccctccggaccagcagaggttgatctttgccggaaaacaattggaggatggtcgtactttggcggattacaacatccagaaggagtcgacccttcacttggtgttgcgtctgcgtggaggtatgcagatcttcgtcaagactttgaccggaaagaccatcacccttgaagtggaaagctccgacaccattgacaacgtcaaggccaagatccaggacaaggaaggtattcctccggaccagcagcgtctcatcttcgctggaaagcagcttgaggatggacgtactttggccgactacaacatccagaaggagtctactcttcacttggtcctgcgtcttcgtggtggtttctaa

SEQ ID NO: 3 - DNA, 5159 bp, Oryza sativa Japonica Group
  Sequence of Rice transketolase(TKT) gene (GenBank number LOC4340025)
catgaaccaaagagtcaaattcaagatggcgtgctgctcttggtcttttgaatgtggcagcagccaggatcaatgcatcctaatatatctatgttggcttagtgaatgactaattaaacacaattaaccccagggatggtcacatgactactgatctcttcaaactgttttaatttcccatgtcagaatctacatctgtacgtacaatttaagctctcctttttttttttttaaaaaaaaaagacgaggttaaactaactaactaagcttgagcatcttctaatgtagtaaggtatataaatcagaagcaaaaacaacatcaaaactgttacaaaaacaagcaaaccatataaataagaatattccacaaaggtagattcctgctgaatagattctatgcccatctgggtctatcttgttatttattctcgtgtggctcggtttcatcatccatcttggcgaatcacagaaaagagcggtttgcaatttatatttggttgaaaagaatggcaattttacatgccaggatcatatacctctgcatctaaaaagttgatttttttgaccccaaattttcaacataaacatcacaatcaaccatggaatgacgccagaaaacaactgtgacggcgacgaccccaacatacacaaggagccaggacaatctttacattatttttatctcttagaaaaaaatatgtcccataacaatttttttttcaaaaggctcaccagaccactaacaaataggcgaaaatatctttaataatatccaatcgtttcctaccctcttccaacggccaggatcatcttgtttgccatatcaacggtcatggctgatatcctcatccaccaacccacctcaccacacccatccccacataaacccagtccatacacgcaaatctaatcaaaaccacatgtatagctgcagaatatatataacaattctatcatatatacatactacaaacttgcactttattattccaagaattgataccatttccagcatcttttttaaacctaactgtagaattttgtgaaagctacaaaatcagtcaggtcaatgttcatgtaaaacgaactaattctcatcagattagtgtgctataaacataagcaaaatatataaagaaattagtaagatcactcctctatgtgaatattttcacacctttgccaaatatgcataaattttttatccaagatgttgcatatacttcctccgtttcacaatgtaagtcactctagcattttccacatttatattgatgttaacgaatctagacagtctagattcgtgtctagcttcattaacatcaatatgaatgtgggaaatgctagaatgacttacattgtgaaacggagggagtacttacctaggttaacatttgttaagttttttttattgtgtcatattttcatcatttgttgataatgctatactttcttttcagtatagactatgtaagtaagtagctatagcttctgttaaaacaaatgtcaggttttatctattatctaaaaacatccaagttaccccctctgcttcatattataagtcgtttatttttttcctaatcaaatattgttaatttcgacaaaatttatagaaaaaattaacaacatctaagatattaaattagttttattaaatctaagttgtatatattttgataatatgattgttgtgttgaaaatactaatatatttttctacatatttagttaaacttaaaaaaagtttgataaaatttttttaaaggatttataatataaaaggagggagtaacatttgttaactacaatataaaaactaatttaaagatcaaataagtacaataactactttataaagattataggttcaataattgcggcgaagaaagtgctcctaatctcaccagccatggcggcttaggatgctggtccatgcaccatgtccacgtccacaccatagccccctcctttttattggctacgagtccccccaaaacgcacccccaagtctatacataacccatcccatctcccaccccacaacctcgccatctccgcctctcctcgccaccacaccactcctctcgccatggccgcgcactccgtcgccgccgcgcacgccaccatcgccgcgcgcgcgggtgccgccgcgccagcgcccgcgccgccggagcgcctcgggttccgcctcagcgcgctcgccggccgcggcctccgctccccgctcccgcctcgccgcggcgcgccatcggcgtccgcgtcgcgccgccgccacaacaaccgcgtgcgcgcggcggcggtcgagacgctcgaggggcaggcggcgacgggggcgctgctcgagaagtcggtgaacacgatccggttcctcgccatcgacgccgtcgagaaggccaactcggggcaccccggcctgcccatggggtgcgcgcccatggggcacatcctctacgacgaggtcatgcggtacaaccccaagaacccctactggttcaaccgcgaccgcttcatcctctccgccggccacggctgcatgctccagtacgccctgctccacctcgccggctacgacgccgtcttggtaagcgcttcccatatccctttgctccttgcatctctctttcgcttctcgagttggttggtttggggcggataaatcgggagtaggttcgtgcaagtttcggatcttgttaggtgaagcagtggaaagtgtggatcttgactgctatttttgtgtggcggcttcgtttttttcctagtaaaagtgtgcaccttttccagtcgtcagagatctgtttagtggaatttaatcttatgtaatacttcctaggaagattagttggaagatgctattaagtcgagcaattgttcactgtaaattctggagtgatcggtgaagtgtttaatatggttgacttcagtttcaactgatttgttcatcgagcttgctgtaactatggccgcatattgctgcaggaggaggacttgaagcaattcaggcaatggggaagcaagactccaggccatcccgagaacttcgagacgcccggagttgaagttaccactggtctgtcatctcaactgaagctcatagcacctttaagtttctatgcttgaatacatacagaactgaatgtttcggtttcttgtttggtttcgttctgtaggacctcttggtcagggtattgcaaatgcagttgggctggcccttgctgagaagcacctggctgctcgtttcaacaagcccgacagcgagattgttgatcactacacgtaattttttttttatcatgaatacctttagttttcactttagttatggtggtgcaaccttggtaacatgattcatggattttgtgcagctactgtattttgggagatgggtgccagatggagggtatctccaatgaagcttgctcgttggctggccattggggtcttggcaagctgattgcattctatgatgacaaccacatttccattgacggagacacagagattgcattcactgaggatgtgagtgctcgctttgaggctcttgggtggcatacaatctgggttaagaatggaaacgatggctatgatgagattcgtgccgccattaaggaagcaaaggcggtaactgacaagccaacactaatcaaggttagattcctgtacattctgttatgctgtgttttgttatagagcttatcatatttagccaaaatgtactctttttaggtgaccaccacaatcggttttggatcacccaacaaggccaactcatacagcgtccacggaagtgctttgggtaccaaagaggttgaagcaaccagagagaaccttggatggccctatgagccattctttgtgcctgaggatgtgaagaggtttgtctattgttgttttggttatctgaatgagatgttgaaatttcatagcactcaccttatggtttatttgcatgggctgttacagccactggagccgccatgtgcctcaaggtgctgcttttgaggctgactggaatgctaagttcgccgagtatgagaagaagtacccagaagatgcagcgaccttgaagagcattgtctcaggggagttgcctgctggctgggctgacgctcttcctgtaagtcttttaccatttctactcataacatttgagctggtatgtgaatgatgacattctaatggtagattacttgatttgtacagaaatacactccagagagccctgcagatgccaccaggaatctgtcacagcagtgcttaaacgcacttgctaaagttgttcctggtcttcttggaggaagtgctgatcttgcatcctccaacatgacattgcttaagatgttcggtgacttccagaaggatacgcctgaggagcgcaatgtccgatttggagtcagggagcatggaatgggcgccatttgcaacggcattgctctgcacagcccaggactcattccatactgtgctactttctttgttttcactgattacatgagagctgccatgaggatctcagccttgtgtgaagccggagttatctatgttatgacccatgactctattggtcttggagaagatggtccaacccatcagcccattgagcacttggtgagcttccgtgcgatgcccaacattctgatgcttcgtcctgctgatggtaacgagactgctggggcatacaaaatcgcggtcctcaacaggaagaggccatccgtccttgctctctccaggcaaaagcttgctcagctgcctggtacctcgattgagggtgttgagaagggtgggtacatcgtctctgacaactcaactggcaacaagcctgacttcattgtgatgagcactggctctgaactagagattgtcgccaaggctgctgatgagttgaggaaggaggggaagactgtccgtgtcgtgtcatttgtttgctgggagcttttcgatgaacagtcggctgagtacaaggagagtgttctccctgaggctgttactgcaagagtcagccttgaagcagggtctactcttggatggcagaagtacgtcggaagcaaaggcaaggctattggcatcgacaaattcggtgcaagtgctcctgctggaaagatctaccaggagtatggcatcaccgcggagaacgtcatcgcaacagcaaagagcctgtaa

SEQ ID NO: 4 - DNA, 5164 bp, Oryza sativa Japonica Group
  Sequence of Rice EPSPS gene (GenBank number LOC4340026)
gctgggtagcctttcaaagccccaaaagattgggtcgggtctctctttcggctctcgtcggtggctgcttcccacaaagaccgccatcagacgtgaatgaactgcaagtctgcaactacgactccagttgctatccccttaaattactttactactacctttattctaagggccggttcggataattgccaaaatcaatcgtaccaaattttaataatattaggatgaaataaatatgttgcgccaaaattttctactgttactgaaatttggtaacaaactaaacacaaccgtatatttatcaattttaccaaatagtggtatggtttaaaatgacattaatctaaataggtcctgaaatataaaaatgcaccaaccatcaatgcatgcagttacattccaaagttctcctaggacacatatgccatatattttcgatggatagccttaatgtctgactactctcttcatcgcaacaaatctaatataatacaaatctgaataggagcttatataaatatatagtagtagaatatgtcataatattacagtggtattccatattggatttgttgattatagaacgacgaaagtatgcgttaacttacaaggttgtttacattatgacgaaatgaagagaaggaacgcacctactgcgaagagctcaacaactacttcctccaaatttccactcaccaactgatcaccaccaattcacttgtgcagtgccacctacccaatttcattccagtggcgtggtacgtaggttccgcatgtcagtgtgacaatcgtacggactgccatgtgtattgtgttcaggtaggtgcactggtcgtggtctcaccatctctcctccagtcgtacggcgccacgtaggcagtacacgtagacggccttcttcctcctccctcgagttgagttggttggtgagagtgagacaccgacggaacggaaggagaaccacgccgcttggatttttcttttttaccttttcaaattttaatttaaaaaataaaaccattttaaaaacttatcttcaaatacaaatcttttaaaaacactaacacgtgacacacagcgggcacgtcacccaaacgggcgtgacaatattgttttgccacaccaatccagctggtgtggacaaaatgttcatatattgaaaataaaatttaaaacaatttatattttttatctatatcattataaaaattgaagatgtttttaccggtattttgttactcatttgtgcatgagtcggtttttaagtttgttcgcttttggaaatacatatccgtatttgagtatgtttttaagttcgttcgttttttgaaatacaaaaggaatcgtaaaataaatctattttaaaaaactcgcatgctaacttgagacgatcgaactgctaattgcagctcataattttccaaaaaaaaatatatccaaacgagttcttatagtagatttcaccttaattaaaacatataaatgttcacccggtacaacgcacgagtatttttataagtaaaattaaaagtttaaaataaataaaaatcccgccaccacggcgcgatggtaaaagggggacgcttctaaacgggccgggcacgggacgatcggccccgaacccggcccatctaaccgctgtaggcccaccgcccaccaatccaactccgtactacgtgaagcgctggatccgcaacccgttaagcagtccacacgactcgactcgactcgcgcactcgccgtggtaggtggcaacccttcttcctcctctatttcttcttcttcctcccttctccgcctcaccacaccaaccgcaccaaccccaaccccgcgcgcgctctcccctctcccctcccaccaaccccaccccatcctcccgacctccacgccgccggcaatggcggcgaccatggcgtccaacgccgcggctgcggcggcggtgtccctggaccaggccgtggcggcgtcggcggcgttctcgtcgcggaagcagctgcggctgcccgccgcggcgcgcggggggatgcgggtgcgggtgcgggcgcgggggcggcgggaggcggtggtggtggcgtccgcgtcgtcgtcgtcggtggcagcgccggcggcgaaggcggaggagatcgtgctccagcccatcagggagatctccggggcggttcagctgccagggtccaagtcgctctccaacaggatcctcctcctctccgccctctccgaggtgagacgcggatcccttcctcttgcgtgaattccatttctggagatgagattttagggggtttattaggtgaggtggctgtgtttgtgaaatcctaggaattatctctcaagtcaatctaacgatgagatataactgaggttctggttttaatcacacactcatataaccaatttattgaaacattttggtttggcataagaaactgcttacgaaggtatgatatcctcctacatgtcaggctactaaattttcacgacggtatgatccactcaaaacaagtttcttaacgagtctggtgaggtctgttatgaaatttgtgtaaactaaggcaactttggaggtttcgcactgtaccaatgttatgtttgaacattttgcaagcagtgctttctcccaaaattatgcaattttgaggctcctctacatcattataattccccaatacattgctctttattcttaatagctttgatcgcgaaatttaacattttaattcttgagctgttattttgtagcatcagtttatcatgagccatgtttggtactaaatatacaatcccttgggtttatttgtttccaagcatgtcattaacttatcttaatgtggacaagaaactgatgcctgcttacattgctattatttcaagcgggtattgatcctttgacatgtgattgatcatttttttttctctggttattagggcacaacagtggtggacaacttgctgaacagtgaggatgttcactacatgcttgaggccctgaaagccctcgggctctctgtggaagcagataaagttgcaaaaagagctgtagtcgttggctgtggtggcaagtttcctgttgagaaggatgcgaaagaggaagtgcaactcttcttggggaacgctggaactgcaatgcgaccattgacagcagccgtgactgctgctggtggaaatgcaacgtatgtttttttttttaatgtttatgaaaatatgtatggaattcatggggtatgttttatgacctttttctttaccatcagttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaaggttagttactcctaaactgcatcctttgtacttctgtatgcacctcaattctttgtcaaccttctgcatttataaggaacattctatgatgcaattcgaccttacactgcacagtaacttgaaatgtttcatgcttaatcaatatgccatattcctgccaagctcaagcgagcaatatttgtttgaatttggtaccatatttttgtatatttgggcattcctttttggtcttgatgtcttcttttgaattagcatttaactgaattacactcaacaggttaagctctctggttccatcagcagtcagtacttgagtgccttgctgatggctgctcctttggcccttggggatgtggagatcgaaatcattgacaaactaatctccattccttacgttgaaatgacattgagattgatggagcgttttggtgtgaaggcagagcattctgatagttgggacagattctatattaagggagggcagaagtacaagtaagcttctacctgccttactgagctgaattattcgggtgtttatgattaactccctaaactaaccctttttcttttctttggcattgacagatctcctggaaatgcctatgttgaaggtgatgcctcaagcgcgagctatttcttggctggtgctgcaatcactggaggcactgtgacagttcaaggttgtggtacgaccagtttgcaggtataactgtagtgcctgttttgacattctaccgtttagtcaagtttagtcagtagtcacatattcagaatatagcacaatctgtattatgccactgttaatcaaatacgcttgacctagagagtgctatataccctagcttaatcttcaaactaaacagttctcttgtggcttgctgtgctgttatgttccctgacctacatgttaatattacagggtgatgtcaaatttgctgaggtacttgagatgatgggagcaaaggttacatggactgacaccagtgtaaccgtaactggtccaccacgtgagccttatgggaagaaacacctgaaagctgttgatgtcaacatgaacaaaatgcctgatgttgccatgacccttgccgttgttgcactcttcgctgatggtccaactgctatcagagatggtaaacattaaggcctattatacctgttctatcatactagcaattactgcttagcattgtgacaaaacaaataaccaaactttcttcaaaataacttagaaatataagaaaggttcgttttgtgtggtaaacagtactactgtagtttcagctatgaagtttgctgctggcaattttctgaacggtttcagctaaattgcatgtttgttcatcatacttatccattgtcttccacagtggcttcctggagagtaaaggaaaccgaaaggatggttgcaattcggaccgagctaacaaaggtaaattcattaggtcccgtgtcctttcattcttcaagtagtttgttcataagttgaattctccttcaatgatgtttaaattcatcatcttcttttttggtgttgtgccagctgggagcatcggttgaagaaggtcctgactactgcatcatcaccccaccggagaagctgaacatcacggcaatcgacacctacgatgatcacaggatggccatggccttctccctcgctgcctgcgccgacgtgcccgtgacgatcagggaccctggttgcacccgcaagaccttccccaactacttcgacgttctaagcactttcgtcaggaactga

SEQ ID NO: 5 - DNA, 3481 bp, Oryza sativa Japonica Group
  Sequence of Rice ubiquitin2 gene (GenBank number LOC4328390)
gaaatcaaaagggaaatgcaccatcttatatctccagtttatatgaacagattggataagatcataagatcaagtggtttatattattttgaggaatataacatggattcatcctaatcactcgtctaggcagtatgtgtattcatgatggatatggtactatactacggagttttttcttcacaaaataacctgttattttgacctccaaccaaacacgaattataccaaaaattgggttatttcatctatagtacaactctattataaacatgcagtaaattatcctacacatataccaaaattcaagtgtaataatcctaatacacagacttaaaaaacaaactatttcctttttaagaaaaggaaaaccatttttttaacggaaggaaaacaaattcgggtcaaggcggaagccagcgcgccaccccacgtcagcaaatacggaggcgcggggttgacggcgtcacccggtcctaacggcgaccaacaaaccagccagaagaaattacagtaaaaaaaaagtaaattgcactttgatccaccttttattacctaagtctcaatttggatcacccttaaacctatcttttcaatttgggccgggttgtggtttggactaccatgaacaacttttcgtcatgtctaacttccctttcagcaaacatatgaaccatatatagaggagatcggccgtatactagagctgatgtgtttaaggtcgttgattgcacgagaaaaaaaaatccaaatcgcaacaatagcaaatttatctggttcaaagtgaaaagatatgtttaaaggtagtccaaagtaaaacttatagataataaaatgtggtccaaagcgtaattcactcaaaaaaaatcaacgagacgtgtaccaaacggagacaaacggcatcttctcgaaatttcccaaccgctcgctcgcccgcctcgtcttcccggaaaccgcggtggtttcagcgtggcggattctccaagcagacggagacgtcacggcacgggactcctcccaccacccaaccgccataaataccagccccctcatctcctctcctcgcatcagctccacccccgaaaaatttctccccaatctcgcgaggctctcgtcgtcgaatcgaatcctctcgcgtcctcaaggtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgccatgggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatgtgtgatgtgcctgttactatctttttgaatacatgtatgttctgttggaatatgtttgctgtttgatccgttgttgtgtccttaatcttgtgctagttcttaccctatctgtttggtgattatttcttgcagatgcagatctttgtgaagacattgaccggcaagactatcaccctcgaggtggagtcctctgacaccatcgataatgtcaaggctaagatccaagataaggagggcatccccccggaccagcagcgtctcatcttcgctggcaagcagctggaggatggcaggacccttgctgactacaacatccagaaggagtcgacccttcaccttgtcctccgcctccgtggtggcatgcagatctttgtcaagactctgaccggcaagactatcacccttgaggtggagtcttctgacaccatcgacaacgtcaaggccaagatccaggacaaagagggcatccccccagaccagcagcgtctcatcttcgccggcaagcagctggaggatggcaggacccttgctgactacaacatccagaaggagtccaccctccaccttgtcctccgcctccgtggtggcatgcagatctttgtcaagacactgaccggcaagaccatcaccctcgaggtggaatcttctgacaccatcgacaacgtcaaggccaagatccaggacaaggagggcattcccccggaccagcagcgtctcatctttgccggcaagcagcttgaggacggcaggacccttgctgactacaacatccagaaggagtcaacgcttcaccttgtcctccgtctcaggggaggcatgcaaatcttcgtgaagactctgaccggcaagaccatcaccctcgaggtggagtcttctgataccatcgacaatgtcaaggccaagatccaggacaaggagggcattcccccggaccagcagcgcctcatctttgctggcaagcagctggaggatggcaggacccttgctgactacaacatccagaaggagtccaccctccaccttgtgctccgccttcgtggtggtatgcagatctttgtcaagaccctcacaggcaagaccatcaccctggaggttgagagctcggacaccatcgacaacgtcaaggccaagatccaggacaaggagggcatccccccagaccagcagcgtctcatcttcgccggcaagcagctcgaggatggccgcaccctcgccgactacaacatccagaaggagtctaccctccacctggtgcttcgtctccgtggtggtatgcagatcttcgtgaagaccttgactgggaagaccatcactttggaggttgagagctccgacaccattgataatgtgaaggccaagatccaggacaaggaggggattcccccagaccagcagcgtctgatcttcgctggcaagcagctggaggatggacgcaccctcgccgactacaacatccagaaggagtccaccctccacctggtgctccgcctccgtggtggtcagtaa

SEQ ID NO: 6 - DNA, 3166 bp, Oryza sativa Japonica Group
  Sequence of Rice HPPD gene (GenBank number LOC4328425)
aaatggtattgtttagaagtttagaaaacgttctgccggacggaatggttccatcccttaaaaaaggacgctgtttcaaattcgaaaaaaagaagagccatcacgataaccagacggttactgacgattttgccgaaggtgattatcgatggtcagtcaacaaagtataaaccctgcttagcggtaataaacacttgatgttttacgtaaaatcttttgaaacttagacggccaacgactatcaaaatatttacatcgaaatcatgaaaaaatgttgtgttcatttgtctttgcaaataatttcaaggcctcataaacttattttaatacaaaataatggctaacgtcacactaaatcaaaatccaatatcaaatgcttttaaggaagcagtagagatatcttcaattgagattttgactgtttgagacagctcattctaccgcttttcatatgtctatcgagaccgaggtaatatatgtggcacaaaacgttttagagaagtgatgtgaaaaacactaccatccatccatttgcaataagaaaatcctccattactactctccccgattcttaatagataacactatcaatttttaaaaatatatatttatcattcatctaattaaggaatataattatcttttatttttttaacttggtttatcattaaaaacacttttaaatatgacttatattttcttatgtttacacatgtttttaaaataaaacaaatgattgaacatataatcacctagtaaaaacaagagctttactccaagttaccacaaaatttgtttccccttccctctaaaaatcagaacagtcaccaacttttctcaaaaaaaaaaaaagtaacaaactcatcagcttcacaaattcaccagcagccaaggcaaccagcaccagaggacaaatcccgtccgcaaccactagacttagcgtccacgtggcgccatctcctccctttatttaaccccccaccaccaactcctccccccacgccgccactgtcatccactcccccacaccccacgacgcgccacgccacgccgcgccgcgccgcgccatgcctcccactcccacccccaccgccaccaccggcgccgtctcggccgctgcggcggcgggggagaacgcggggttccgcctcgtcgggcaccgccgcttcgtccgcgccaacccgcggagcgaccggttccaggcgctcgcgttccaccacgtcgagctctggtgcgccgacgccgcgtccgccgcgggccggttcgccttcgccctgggcgcgccgctcgccgccaggtccgacctctccacggggaactccgcgcacgcctccctcctcctccgctccgcctccgtcgcgttcctcttcaccgccccctacggcggcgaccacggcgtcggcgcggacgcggccaccaccgcctccatcccttccttctccccaggcgccgcgcggaggttcgccgcggaccacggcctcgcggtgcacgccgtggcgctgcgcgtcgccgacgcggccgacgccttccgcgccagcgtcgcggccggtgcgcgcccggcgttccagcccgccgacctcggcggtggcttcggcctcgcggaggtggagctctacggcgacgtcgtgctccgcttcgtcagccacccggacggcgccgacgcgcccttcctcccgggtttcgagggcgtcagcaacccgggcgccgtggactacggcctccgccggttcgaccacgtcgtcggcaacgtgccggagctcgctccggtagccgcgtacatctccgggttcaccgggttccacgagttcgccgagttcaccgccgaggacgtgggcaccgccgagagcggcctcaactcggtggtgctcgccaacaacgcggagaccgtgctgctgccgctcaacgagccggtgcacggcaccaagcggcggagccagatacagacgtacctggaccaccacggcggcccgggggtgcagcacatcgcgctggccagcgacgacgtgctcgggacgctgagggagatgcgggcgcgctccgccatgggcggcttcgagttcttggcgccgccgccgcccaactactacgacggcgtgcggcggcgcgccggggacgtgctctcggaggagcagatcaacgagtgccaggagctcggggtgctcgtggacagggatgaccagggggtgttgctccagatcttcaccaagccagtaggagacaggtaaaatcctcacctctttcatgatgaaaatggcttatgaattcagatttgcagttatttgttggcacatagcatcgattaggcgcagaaaggtgtcaagcattatgaaattaatccagaatgcttgaataatacagtataatatatgatagtgagctctgtgatactccatggatactctttatgtgtctccatgaatccatgatgcgcctttctgaagattgtgacactagaaagggaataaagctgaatgtgcataggaaaaaaatgaaaagccaatgtgtgtctgtttatgccttcttgcaagcatatcccagttcctttttgccggcatgttgtaatgcagatagccagccacatatagctacttaattagtgagtactccctctcacaatgtaagtcattctagtattttccacattcatattgatgctaatctatctagattcattagcatcaatatgaatatgggaaatactagaatgacttacattgtgaaacggaggaagtattacttactacatctaaggtccatggattcctttttttacaaaagaaagaaagaatcttatggcaactccatcagcataaaccagcaatgctgctgggaacaacttaaactttaggttcaggaggttgtaattgtctttaagcttaatagtctgattcagtcagtattctaatttctgctgcatctttgctattgttatttcctctctgtgactccaaatctaactggatcagctatttcactcaggccaacctttttcttggagatgatacaaaggattgggtgcatggagaaggatgagagtgggcaggagtaccagaagggcggctgcggcgggtttgggaagggcaacttctcggagctgttcaagtccattgaggagtatgagaaatcccttgaagccaagcaagcccctacagttcaaggatcctag

SEQ ID NO: 7 - DNA, 4311 bp, Oryza sativa Japonica Group
  Sequence of Rice PPO gene (GenBank number LOC4327918)
caaaaaaaattattcaaaaggaagcaggcagggcagcacattgtgagggaatcaggcggcggcgcacagttctctcccttggcaccctcctttccctcatatgcaatggactcaaaagggcggctgcagcgtatgccggtggattaggtttcgagggtggatccagtgagagcaaaggtggtggggtgacccaaggacgctgcttcgctcaagtccacatgtgcttcgcctcaaggccaggatgccgccgccctactcgagactaggtagctgacttgagactaggccgttgtcgcgcgtcggggccgcccagccgctgctccaccttgggtcacgctgtcactctatctcaaggctgggccatcgccactatgcttgaagccgcactaccgtcccacctcaaggccggcaccctgtcactccacctcgaggctacctagacgttattttgttccgctgctacgccttagagacaccccactgccgctctaccttgggctatgccgtcgctctttctccaaggcgcgctgctacaactccgccctgagtctccatgtgcctgtgtggagaaaggagagggaagaaagaaagaaaggagaaagaagtgggaggaatcactaacatgtgagctctgtatattgatttggtcagttggattaggtcaacgagccatccaagagaaaacaacccttaaaatcattgagaaaatcatttttaactgttttaatagttaagagatgaagatatatttttttttatagaaagagatgaagatatccgatatccaatatgataggaatcagaaaacgtatatttttgagggagtcaatgcggacttattcccaccagaaactgcagcatattggacgcagtggttccttccgcggtagctgcgcccgcgcccacgggaatgcgattcccagcccattccacccccttcccattccggtgggccattccgaatccaaccaaccaaccaaccatcgaacccgcacccgcatcgtatccactcctctccagtctccccgccgctccgcatcccgcagccgctcgtcagcgacggacatggccgccgccgccgcagccatggccaccgccacctccgccacggcagcgccgccgctccgcattcgcgacgccgcgaggaggacccgccgacgcggccacgttcgctgcgccgtcgccagcggcgcggccgaggcgcccgcggcgcccggggcgcgggtgtcggcggactgcgtcgtggtgggcggcggcatcagcgggctctgcaccgcgcaggcgctggccacaaagcacggcgtcggcgacgtgctcgtcacggaggcccgcgcccgccccggcggcaacatcaccaccgccgagcgcgccggcgagggctacctctgggaggaggggcccaacagcttccagccttccgaccccgtcctcaccatggccgtacgtcttcttgctccccttctcttctgattctctcgcggcggagacgacatgaatgggaatggtggtgcatggattggggcgcgcaggtggacagcgggctcaaggacgatctcgtgttcggggaccccaacgcgccgcggttcgtgctgtgggaggggaagctaaggccggtgccgtccaagcccggcgacctgccgttcttcgacctcatgagcatccccggcaagctcagggccggccttggcgcgctcggcgttcgagcgccacctccagtttgtgtgctctccccgctgtgcattcttgattcacttgtgaaattcgattgtgctgagcgtttccggcgaaggtttcaggggcgtgaggagtcggtggaggacttcgtgcggcgcaacctcggcgcggaggtctttgagcgcctcattgagcctttctgctcaggtgtttattgtagtgtgcaattgctgttttgtttttgatgattcagataagaatacggtgatttcggtgcttaggtgtgtatgctggtgatccttcaaagctcagtatgaaggctgcatttgggaaggtgtggaggctggaggatactggaggtagcattattggtggaaccatcaaaacaatccaggagagggggaaaaaccccaaaccgccgagggatccgtgagtgagaaattgccttctttgttggattaattgtccattgtgttacactgatatgccttcaccatttttagccgccttccaacgccaaaggggcagacagttgcatctttcaggaagggtctgactatgctcccggatgctattacatctaggtttgttatcattgtctttgtaatttacctagttcttcaactatggatattaggtgctgtagattgttcagatagatgcacattgtacaacaatctaggtagattgattgctatggcttgttgaattaactgtttcacttgcatcattgcctcagcacatatgaagcatatggatagattcttcaatcatttatccctcaataacacaattttgacaccagtgcttctcctttttttcatcctgcttcatctggctatccacaatataattaagcatacaaaagaggcactctttgatggacaattcatagtgttgtgggttaatattcatttgcattctttgagggacaattcgttacaccctaacatgaactagtaatgattggggtgcttaaccatttgttctgcatttcctccattttcaggttgggtagcaaagtcaaactttcatggaagttgacaagcattacaaagtcagacaacaaaggatatgcattagtgtatgaaacaccagaaggggtggtctcggtgcaagctaaaactgttgtcatgaccatcccatcatatgttgctagtgatatcttgcggccactttcagtaagttatatatatttaattaactttctgttcccaaaatacactgcagcacttcattgcttcctgaggtcctcgattcatttttcggtagacaggaagtagtattcatttgcactttttaagggattaattcaacatatccactggaaatatacatatcctacacatcctgtcaacatacttgctaaacagcattttgtttgagttgactggcatctcagcagccaattactatctttaggggacaagccacattcttaataaatcctgtcggaaatcactttttgatttttatagattagtgttgtcatagaatttggcttggtgatctacttggtaaggttaactgattcacaagtcggacaatttcttcaccaatctagcagtatgtttaagtgtgttggtacttaattctaaatgtcctgcgcatggtaacatatcatatgcaaaaattcctcagtaaccgaaatttatactgtaagttttaactgtctttacactgttaattttagacatacttcttccttgcttgttcattgaacttgtttcccccttccacagagtgatgcagcagatgctctgtcaatattctattatccaccagttgctgctgtaactgtttcatatccaaaagaagcaattagaaaagaatgcttaattgacggagagctccagggtttcggccagctgcatccgcgtagtcagggagttgagactttaggtacttataggaattcaaccttattattcttctaacatataaatgaactaatctttcttgtctagtttgcatttattgtggattaagtttggttatattgttcttacaagtttgtggtattattttgtataggaacaatatatagctcatcactctttccaaatcgtgctccagctggaagggtgttacttctgaactacataggaggttctacaaatacagggattgtttccaaggtatcgctgtcaagttgtttattttgcgactatatgattacagtatcctgtttttcaactccagctgctgttagactgtcataataaatctgctactacatgtttgcacactatttgactgcatttaaaaactcagatagcctatatttttagttgcctgctactgggtgtatttctaatgatcccatcatgtttgcagactgaaagtgagctggtagaagcagttgaccgtgacctcaggaagatgctgataaatcctaaagcagtggaccctttggtccttggcgtccgggtatggccacaagccataccacagttcctcattggccatcttgatcatcttgaggctgcaaaatctgccctgggcaaaggtggttatgatggattgttcctcggagggaactatgttgcaggagttgccctgggccgatgcgttgaaggtgcatatgagagtgcctcacaaatatctgactacttgaccaagtacgcctacaagtga

SEQ ID NO: 8 - DNA, 2463 bp, Oryza sativa Japonica Group
  Sequence of Rice CP12 gene (GenBank number LOC4325791)
tttaaatcgtatttctacttggactctcctttccttttctaattttggattttttttttgaattttgattaatctcgtattgggttcttatatggaaacttctttcaatattgcttattttcaattccgaatttcagctatttttaaatcgtatttctacttggactctccttttctttttttctttttcttcgattaatgtgggaatttctagcccccacagcgaacgtggtgactattttcaaagctgttttaataatataatagattttattttagggcctgttcagattgtagccaaaataaaccttatcaaaatttgacaataccaaaattttgtcaagttgacaatattgccaaaattttggcaggatttcttacgtatttatcaaatttggcaacaaactaaacatagatatttttttggcaactttaccaaaaaagttgtatgattgaaaatgacatcaatctgaacagccccttagaacttagaactacaatctgaacagccccttagaacttagaactacagtggaatccatcttggcatatgtacttggcagggaattttttttacatttttattattaaaaaattacaaaattttttttcgaaaatttacaaatctagacgcctaacatcctttaggagggcgtttttttaaatcgcccttctaaagggcgttaagggacctaaatgcaaaatttttatttgtattcgaaccctttccaccagttttaattgcttaaaagctaataatgcttatttgacactccaaataattttaaatgaaaaggtaataaactacaaagttgtagatctcatcgagatctatatcttttttataaagtttatctccatccaatgtcgtttgaaatgtagttctgagaatttttttaaataggttttagattttgtaacgaatatttggacatctaaacgatcttaaatgaaagttgttaattacaaacttgtagatcttctccagctctacaattttgatataaactttaatttcatatggctttatatgatatagttttaaattgtaaaatcatagaggtagcaagctatgatggaaaatttgtatttaggtccctaacgccctctggaagggtgatttgaaaaaagccctctaggaggacgtgaggcacgactagatttgtaaattttcggaatgaaaaattatttttaaatattttaatcaaaaaatgtaaaaataaaaaaaattctcggcagggtggcagcatgggcctaaggcccagtcaactgtgggcctataagcgactaatccggctgtaactgggccttgcaagaggcttgtcttgttggtccgaactcaggaagtccaggttgcggggacaacttcaaggccatctggtttccacttctcttaccacctcaattccgctcttgatccgagctagcttagtcccaatctaaaaactttacaaagaaagaaccatacgcacctattgggcaaaatgaaaaataatttgctactcaccaaataatttgagcacctctgcacctgtacactaaataactctgttccaccaaaatagttgagatatctaggacgtttcattttgtccgttcttcaccaaacttttccatagtatctcagatattttcgagaccgaaagtgatctttctggccttagaccgagttcacttccctacaagccattctttgctggcacaacacgaacctctacatcaatttcgtatccaacctgaacttctgcatacatgtacacacccacagtcatctgctcatgttttcacggtcaaattaaaactgcttctctcaccttagattcacccaagggaaaagaaaaagatctcctttgccaagtccccatttcgcatgaaatatctcaaaatacagcccacgtggcacacgacgattggctgaggaggcgataagaaacgagtgcacgtcgtcgaatcctctctccccttctcccccaccccacggagctatatatatataaaccccatctcttcaatccgtgcaacgaacgcctcgtcgcaacagctacaaacgcccacatcacacgcagaaatccgcatcaacagccagccatggcgtccacgctgaccaacgtcggcctgtctaccccggcggcggcggcgtcgtccctcgttaggccggtcgccggagctggacgcgtggtgtttccccgtgttggccgcggcgggttcgcggcggtgagggcgagcgggccggcgacgccgccggacatctcggacaagatgtcggagagcatcgacaaggcgaaggaggcgtgcgcggaggacacggcgagcggcgagtgcgcggcggcgtgggacgaggtggaggagctgagcgcggcggcgagccacgcgcgcgacaagctcaaggagacctccgacccgctcgaggcctactgcaaggacaacccggagaccgacgagtgccgcacctacgacaactga

SEQ ID NO: 9 - DNA, 744 bp, Artificial Sequence
  Sequencing result of doubling resulting from pQY002066 transforming protoplast
ttcaagtccataggcgaggcgaccggcccgcggcggacgcggcgtcggcgcaccagagctcgacgtggtggaacgcgagcgcctggaaccggtcgctccgcgggttggcgcggacgaagcggcggtgcccgacgaggcggaaccccgcgttctcccccgccgccgcagcggccgagacggcgccggtggtggcggtgggggtgggagtgggaggcatggcgcggcgcggcgcggcgtggcgtggcgcgtcgtggggtgtgggggagtggatgacagtggcggcgtggggggaggagttggtggtggggggttaaataaagggaggagatggcgccacgtggacgctaagtctagtggttgcggacgggatttgtcctctggtgctggttgccttggcagatagggtaagaactagcacaagattaaggacacaacaacggatcaaacagcaaacatattccaacagaacatacatgtattcaaaaagatagtaacaggcacatcacacatgtattcagataggtagtaacagcacacgtatcctgaacttcataaataagtagaatgaactagtggcaataacttttcacacagaacatctcccttgttttcaaatatgtagtaacagcacacgtatcctgtacttcaaaacttagtagaatgaactagtggcagtaatctttcacacagaaagaatgaactagtggcaataatctttcacacagaatgttggttgatggatag

SEQ ID NO: 10 - DNA, 603 bp, Artificial Sequence
  Sequencing result of doubling resulting from pQY002068 transforming protoplast
caacgtgccttaggcgaggcgaccggcccgcggcggacgcggcgtcggcgcaccagagctcgacgtggtggaacgcgagcgcctggaaccggtcgctccgcgggttggcgcggacgaagcggcggtgcccgacgaggcggaaccccgcgttctcccccgccgccgcagcggccgagacggcgccggtggtggcggtgggggtgggagtgggaggcatggcgcggcgcggcgcggcgtggcgtggcgcgtcgtggggtgtgggggagtggatgacagtggcggcgtggggggaggagttggtggtggggggttaaataaagggaggagatggcgccacgtggacgctaagtctagtggttgcggacgggatttgtcctctggtgctggttgccttggagatagggtaagaactagcacaagattaaggacacaacaacggatcaaacagcaaacatattccaacagaacatacatgtattcaaaaagatagtaacaggcacatcacacatgtattcagataggtagtaacagcacacgtatcctgaacttcagaaataagtagaatgaactagtggcaataacttttccccccaaaaacctgtc

SEQ ID NO: 11 - DNA, 477 bp, Artificial Sequence
  Sequencing result of reversion resulting from pQY002062 transforming protoplast
gggcggatttcgacggaggggcggagaggaggaggatcctgttggagagcgacttggaccctggcagctgaaccgccccggagatctccctgatgggctggagcacgatctcctccgccttcgccgccggcgctgccaccgacgacgacgacgcggacgccaccaccaccgcctcccgccgcccccgcgcccgcacccgcacccgcatccccccgcgcgccgcggcgggcagccgcagctgcttccgcgacgagaacgccgccgacgccgccacggcctggtccagggacaccgccgccgcagccgcggcgttggacgccatggtcgccgccattgcggtgtggtggcgaggagaggcggagatggcgaggttgtggggtgggagatgggatgggttatgtatagacttgggggtgcgttttggggggactcgtagccaataaaaaggagggggctatggtgtggccgtttggacat

SEQ ID NO: 12 - DNA, 670 bp, Artificial Sequence
  Sequencing result of deletion resulting from pQY002062 transforming protoplast
ttttcgttggaggggcggagaggaggaggatcctgttggagagcgacttggaccctggcagctgaaccgccccggagatctccctgatgggctggagcacgatctcctccgccttcgccgccggcgctgccaccgacgacgacgacgcggacgccaccaccaccgcctcccgccgcccccgcgcccgcacccgcacccgcatccccccgcgcgccgcggcgggcagccgcagctgcttccgcgacgagaacgccgccgacgccgccacggcctggtccagggacaccgccgccgcagccgcggcgttggacgccatggtcgccgccattggacgagtcggaatcgcagaccgataccaggatcttgccatcctatggaactgcctcggtccatggccgcgcactccgtcgccgccgcgcacgccaccatcgccgcgcgcgcgggtgccgccgcgccagcgcccgcgccgccggagcgcctcgggttccgcctcagcgcgctcgccggccgcggcctccgctccccgctcccgcctcgccgcggcgcgccatcggcgtccgcgtcgcgccgccgccacaacaaccgcgtgcgcgcggcggcggtcgagacgctcgaggggcaggcggcgacgggggcgctgctcgagaagtcggaaacccccgaatccaaa

SEQ ID NO: 13 - DNA, 777 bp, Artificial Sequence
  Sequencing result of reversion resulting from pQY002093 transforming protoplast
gaacgatgcgagccccacgctcgaccgccgccgcgcgcacgcggttgttgtggcggcggcgcgacgcggacgccgatggcgcgccgcggcgaggcgggagcggggagcggaggccgcggccggcgagcgcgctgaggcggaacccgaggcgctccggcggcgcgggcgctggcgcggcggcacccgcgcgcgcggcgatggtggcgtgcgcggcggcgacggagtgcgcggccatggcgagaggagtggatggtcacctgactaccggtcccctcaaactggtttaatttcccctggcaaaatccacctccggacctacaatttaagctcccctttttttttttaaaaaaaaaaaaaaagagggtaaaccaactaacctagcttggaccttttttaatggagtaagggatttaaaacaaaaacaaaaaaaaaatccaaactggtaaaaaaacaaacaaaccatttaaataaaaattttccccaaagggaaattcctggcgaaaaaaatctatgcccctctgggtctatcttggtattttttccccggggctccgtttcatccttcattttggcgaatacaaaaaaaaccgtttgaattttttttggttgaaagaatggcaatttactggccaggatcatgtactctgcatctaagaattgattttttgaccccaaatttcaacttagcctccatcgcagtagtgcgcacacacaggctgaaggtgactcttagacccaatgtcactatctcagcaatatgcagagagaatgacccaa

SEQ ID NO: 14 - DNA, 635 bp, Artificial Sequence
  Sequencing result of deletion resulting from pQY002093 transforming protoplast
agaacgatgcgcagcctcggcgtctcgaccgccgccgcgcgcacgcggttgttgtggcggcggcgcgacgcggacgccgatggcgcgccgcggcgaggcgggagcggggagcggaggccgcggccggcgagcgcgctgaggcggaacccgaggcgctccggcggcgcgggcgctggcgcggcggcacccgcgcgcgcggcgatggtggcgtgcgcggcggcgacggagtgcgcggccatggcgagaggagtgtggtgggtaccctttcaaacccccaaaagagtgggtcgggtctctctttcggctctcggcgggggctgcttcccacaaagaccgccatcagacgtgagtgaactgcaagtctgcaactaccactccaggtgctctccccttaaattactttactactacctttattctaggggccggttcagataattgccaaaatcactcgcaccattttttaataatattaggatgaaatatatatgtcgcgccaaatttttctactgttactgaaatttggacacaaactacacaccaccgtatatttatcttttttaccaaataggggtatggtttaaaatgactttaatctaaataggccctgaaatataaaagcgccccacccctca

SEQ ID NO: 15 - DNA, 652 bp, Artificial Sequence
  Sequencing result of reversion resulting from pQY002095 transforming protoplast
tttgagtagactcgccggcgcgctcggcggtggtgatgttgccgccggggcgggcgcgggcctccgtgacgagcacgtcgccgacgccgtgctttgtggccagcgcctgcgcggtgcagagcccgctgatgccgccgcccaccacgacgcagtccgccgacacccgcgccccgggcgccgcgggcgcctcggccgcgccgctggcgacggcgcagcgaacgtggccgcgtcggcgggtcctcctcgcggcgtcgcgaatgcggagcggcggcgctgccgtggcggaggtggcggtggccatggctgcggcggcggcggccatgtccgtcgctgacatgcggatttctgcgtgtgatgtgggcgtttgtagctgttgcgacgaggcgttcgttgcacggattgaagagatggggtttatatatatatagctccgtggggtgggggagaaggggagagaggattcgacgacgtgcactcgtttcttatcgcctcctcagccaatcgtcgtgtgccacgtgggctgtattttgagatatttcatgcgaaatggggacttggcaaaggagatctttttcttttcccttgggtgaatctaaggtgagagaagcagttttaatttgaccgtgaaaacatgagccaaaaggaaaaaagg

SEQ ID NO: 16 - DNA, 506 bp, Artificial Sequence
  Sequencing result of deletion resulting from pQY002095 transforming protoplast
acgcgtggttcttccggtagctgcgcccgcgcccacgggaatgcgattcccagcccattccacccccttcccattccggtgggccattccgaatccaaccaaccaaccaaccatcgaacccgcacccgcatcgtatccactcctctccagtctccccgccgctccgcatcccgcagccgctcgatgcggatttctgcgtgtgatgtgggcgtttgtagctgttgcgacgaggcgttcgttgcacggattgaagagatggggtttatatatatatagctccgtggggtgggggagaaggggagagaggattcgacgacgtgcactcgtttcttatcgcctcctcagccaatcgtcgtgtgccacgtgggctgtattttgagatatttcatgcgaaatggggacttggcaaaggagatctttttcttttcccttgggtgaatctaaggtgagagaagcagttttaatttgaccgtgaaaacatagcccagaatgacaaaataaccttt

SEQ ID NO: 17 - DNA, 614 bp, Artificial Sequence
  Sequencing result of reversion resulting from pQY002098 transforming protoplast
gcatcgtccggccgtcggcggtggtgatgttgccgccggggcgggcgcgggcctccgtgacgagcacgtcgccgacgccgtgctttgtggccagcgcctgcgcggtgcagagcccgctgatgccgccgcccaccacgacgcagtccgccgacacccgcgccccgggcgccgcgggcgcctcggccgcgccgctggcgacggcgcagcgaacgtggccgcgtcggcgggtcctcctcgcggcgtcgcgaatgcggagcggcggcgctgccgtggcggaggtggcggtggccatggctgcggcggcggcggccatgttgtgggcgtttgtagctgttgcgacgaggcgttcgttgcacggattgaagagatggggtttatatatatatagctccgtggggtgggggagaaggggaaagaggattcgacgacgtgcactcgtttcttatcgcctcctcagccaatcgtcgtgtgccacgtgggctgtattttgagatatttcatgcgaaatggggacttggcaaaggagatctttttcttttcccttgggtgaatctaaggtgagagaagcagttttaatttgaccgtgaaaacatgagcccaaattgacaaatccc

SEQ ID NO: 18 - DNA, 574 bp, Artificial Sequence
  Sequencing result of HPPD doubling in QY2091-13
ggatacgtgtgctgttactacctatctgaatacatgtgtgatgtgcctgttactatctttttgaatacatgtatgttctgttggaatatgtttgctgtttgatccgttgttgtgtccttaatcttgtgctagttcttaccctatctgccaaggcaaccagcaccagaggacaaatcccgtccgcaaccactagacttagcgtccacgtggcgccatctcctccctttatttaaccccccaccaccaactcctccccccacgccgccactgtcatccactcccccacaccccacgacgcgccacgccacgccgcgccgcgccgcgccatgcctcccactcccacccccaccgccaccaccggcgccgtctcggccgctgcggcggcgggggagaacgcggggttccgcctcgtcgggcaccgccgcttcgtccgcgccaacccgcggagcgaccggttccaggcgctcgcgttccaccacgtcgagctctggtgcgccgacgccgcgtccgccgcgggccggttcgccttcgccctgggcgcgccgctcgccgccaggtccgacctctccacggg

SEQ ID NO: 19 - DNA, 551 bp, Artificial Sequence
  Sequencing result of HPPD doubling in QY2091-20
aggatacgtgtgctgttactacctatctgaatacatgtgtgatgtgcctgttactatctttttgaatacatgtatgttctgttggaatatgtttgctgtttgatccgttgttgtgtccttaatcttgtccaaggcaaccagcaccagaggacaaatcccgtccgcaaccactagacttagcgtccacgtggcgccatctcctccctttatttaaccccccaccaccaactcctccccccacgccgccactgtcatccactcccccacaccccacgacgcgccacgccacgccgcgccgcgccgcgccatgcctcccactcccacccccaccgccaccaccggcgccgtctcggccgctgcggcggcgggggagaacgcggggttccgcctcgtcgggcaccgccgcttcgtccgcgccaacccgcggagcgaccggttccaggcgctcgcgttccaccacgtcgagctctggtgcgccgacgccgcgtccgccgcgggccggttcgccttcgccctgggcgcgccgctcgccgccaggtccgacctctcca

SEQ ID NO: 20 - DNA, 155 bp, Artificial Sequence
  Sequencing result of PPO1 reversion in QY2234-20
tctctctcccttctcccccaccccacggagctatatatatataaaccccatctcttcaatccgtgcaacgaacgcctcgtcgcaacagctacaaacgcccacaagcaacgaaggaaatgggcggccgccgccgaaccctgggccccggcccctcc

SEQ ID NO: 21 - DNA, 447 bp, Artificial Sequence
  Sequencing result of PPO1 reversion in QY2234-205
cccttctcccccaccccacggagctatatatatataaaccccatctcttcaatccgtgcaacgaacgcctcgtcgcaacagctacaaacggacatggccgccgccgccgcagccatggccaccgccacctccgccacggcagcgccgccgctccgcattcgcgacgccgcgaggaggacccgccgacgcggccacgttcgctgcgccgtcgccagcggcgcggccgaggcgcccgcggcgcccggggcgcgggtgtcggcggactgcgtcgtggtgggcggcggcatcggcgggctctgcaccgcgcaggcgctggccacaaagcacggcgtcggcgacgtgctcgtcacggaggcccgcccccgccccggcggcaacatcaccaccgccgagcgcgccggcgagggctacctctgggaggaggggcccaacagcttccagccttcc

SEQ ID NO: 22 - DNA, 463 bp, Artificial Sequence
  Sequencing result of PPO1 reversion in QY2234-207
cccttctcccccaccccacggagctatatatatataaaccccatctcttcaatccgtgcaacgaacgcctcgtcgcaacagctacaaacgcccacagtcagcgacggacatggccgccgccgccgcagccatggccaccgccacctccgccacggcagcgccgccgctccgcattcgcgacgccgcgaggaggacccgccgacgcggccacgttcgctgcgccgtcgccagcggcgcggccgaggcgcccgcggcgcccggggcgcgggtgtcggcggactgcgtcgtggtgggcggcggcatcagcgggctctgcaccgcgcaggcgctggccacaaagcacggcgtcggcgacgtgctcgtcacggaggcccgcgcccgccccggcggcaacatcaccaccgccgagcgcgccggcgagggctacctctgggaggaggggcccaacagcttccagccttcc

SEQ ID NO: 23 - DNA, 388 bp, Artificial Sequence
  Sequencing result of PPO1 reversion in QY2234-580
cccttctcccccaccccacggagctatatatatataaaccccatctcttcaatccgtgcaacgaacgcctcgtcgcaacaggtacatggccgccgccgccgcagccatggccaccgccacctccgccacggcagcgccgccgctccgcattcgcgacgccgcgaggaggacccgccgacgcggccacgttcgctgcgccgtcgccagcggcgcggccgaggcgcccgcggcgcccggggcgcgggtgtcggcggactgcgtcgtggtgggcggcggcatcagcgggctctgcaccgcgcaggcgctggccacaaagcacggcgtcggcgacgtgctcgtcacggaggcccgcgcccgccccggcggcaacatcaccaccgccgagcgc

SEQ ID NO: 24 - DNA, 417 bp, Artificial Sequence
  Sequencing result of PPO1 reversion in QY2234-H5-159
cccttctcccccaccccacggagctatatatatataaaccccatctcttcaatccgtgcaacgaacgcctcgtcgcaacagctacaaacggacatggccgccgccgccgcagccatggccaccgccacctccgccacggcagcgccgccgctccgcattcgcgacgccgcgaggaggacccgccgacgcggccacgttcgctgcgccgtcgccagcggcgcggccgaggcgcccgcggcgcccggggcgcgggtgtcggcggactgcgtcgtggtgggcggcggcatcagcgggctctgcaccgcgcaggcgctggccacaaagcacggcgtcggcgacgtgctcgtcacggaggcccgcgcccgccccggcggcaacatcaccaccgccgagcgcgccggcgagggctacctctgg

SEQ ID NO: 25 - DNA, 430 bp, Artificial Sequence
  Sequencing result of PPO1 reversion in QY2234-H5-232
ccttctcccccaccccacggagctatatatatataaaccccatctcttcaatccgtgcaacgaacgcctcgtcgcaacagctacaaacgcccgtcagcgacggacatggccgccgccgccgcagccatggccaccgccacctccgccacggcagcgccgccgctccgcattcgcgacgccgcgaggaggacccgccgacgcggccacgttcgctgcgccgtcgccagcggcgcggccgaggcgcccgcggcgcccggggcgcgggtgtcggcggactgcgtcgtggtgggcggcggcatcagcgggctctgcaccgcgcaggcgctggccacaaagcacggcgtcggcgacgtgctcgtcacggaggcccgcgcccgccccggcggcaacatcaccaccgccgagcgcgccggcgagggctacctctggg

SEQ ID NO: 26 - DNA, 424 bp, Artificial Sequence
  Sequencing result of PPO1 reversion in QY2234-H5-263
ataaaccccatctcttcaatccgtgcaacgaacgcctcgtcgcaacagctacaaacgcccacagtcagcgacggacatggccgccgccgccgcagccatggccaccgccacctccgccacggcagcgccgccgctccgcattcgcgacgccgcgaggaggacccgccgacgcggccacgttcgctgcgccgtcgccagcggcgcggccgaggcgcccgcggcgcccggggcgcgggtgtcggcggactgcgtcgtggtgggcggcggcatcagcgggctctgcaccgcgcaggcgctggccacaaagcacggcgtcggcgacgtgctcgtcacggaggcccgcgcccgccccggcggcaacatcaccaccgccgagcgcgccggcgagggctacctctgggaggaggggcccaacagcttccag

SEQ ID NO: 27 - DNA, 327 bp, Artificial Sequence
  Sequencing result of new HPPD gene resulting from rice protoplast sample transformed by pQY2658 vector
tcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatccccaccaccaactcctccccccacgccgccactgtcatccactcccccacaccccacgacgcgccacgccacgccgcgccgcgccgcgccatgcctcccactcccacccccaccgccaccaccggcgccgtctcggccgctgcggcggcgg

SEQ ID NO: 28 - DNA, 250 bp, Artificial Sequence
  Sequencing result of new OsCATC gene resulting from rice protoplast sample transformed by pQY2654 vector
tggaggctgtcctacgacacccatccctttcccgcctcaaaccccccaaccccaatgctcagagaactccagccctctccatcaccgtccctttccggcttcgtctccccaaccggcggctcaccgccgtacaaggtaacgaacgtgctctgctctgcttctggggcgaggggtttgagagtgcgttggttttttgtggttttgatgcggaatttggtgtgatgcgcgcagcaccgcccgtcgagctcgt

SEQ ID NO: 29 - DNA, 226 bp, Artificial Sequence
  Sequencing result of new OsGLO3 gene resulting from rice protoplast sample transformed by pQY2655 vector
ttagttcaggaaaggtaaatgatctgcactcacacacatgtaccattttctgtttttttttccccagccgatcataaggtgcaggagatttacaaggacaaggcttctccgatgcttggtggcaagaatggagctaatcacaaacgtctccgagtatgagcagcttgcaaagcagaagctgccgaagatgatctacgactactacgcctctggtgcagaagatcaa

SEQ ID NO: 30 - DNA, 327 bp, Artificial Sequence
  Sequencing result of new PPO2 gene from rice callus sample QY1386/818-28#
tttaggatttcttgcaattccattttggcacttttcttctgacctatttctctggctgctgcttcctgataatcgaccagttctctagtcttgctccctgcactcctcccttcccatactgatcccaaatcagatgctctctcctgccaccaccttctcctcctcctcctcctcctcgtcgccgtcgcgcgcccacgctcgcgctcccacccgcttcgcggtcgcagcatccgcgcgcgccgcacggttccgccccgcgcgcgccatggccgcctccgacgacccccgcggcgggaggtccgtcgccgtcgtcggcgccggcgtcag

SEQ ID NO: 31 - DNA, 370 bp, Artificial Sequence
  Sequencing result of new PPO2 gene from rice callus sample QY1386/818-62#
tttaggatttcttgcaattccattttggcacttttcttctgacctatttctctggctgctgcttcctgataatcgaccagttctctagtcttgctccctgcactcctccctcctccatctccggcaaagacaacaagtaaatccccatccatagctatccaagagccccaaatcagatgctctctcctgccaccaccttctcctcctcctcctcctcctcgtcgccgtcgcgcgcccacgctcgcgctcccacccgcttcgcggtcgcagcatccgcgcgcgccgcacggttccgccccgcgcgcgccatggccgcctccgacgacccccgcggcgggaggtccgtcgccgtcgtcggcgccggcgtcag

SEQ ID NO: 32 - DNA, 322 bp, Artificial Sequence
  Sequencing result of new PPO2 gene from rice callus sample QY1387/818-15#
tttcttgcaattccattttggcacttttcttctgacctatttctctggctgctgcttcctgataatcgaccagttctctagtcttgctccctgcactcctcccttcctatactgaacccccatctcatatgctctcttgtgaccaccttctcttcttcttcttctcctcctcctccgcgtcgcgcccccactctcgctctcccccccttttcgcgcgcacaatccgcgcgcgccgcacgcgtcccccccgcgcgcgccacgtgggcgtccgacaacacccgcgcggcgagaggcgcgtccgcgtccgccgccgcggcgtcgg

SEQ ID NO: 33 - DNA, 323 bp, Artificial Sequence
  Sequencing result of new PPO2 gene from rice callus sample QY1387/818-64#
tttcttgcaattccattttggcacttttcttctgacctatttctctggctgctgcttcctgataatcgaccagttctctagtcttgctccctgcactcctccctcctctatccaagagccccaaatcagatgctctctcctgccaccaccttctcctcctcctcctcctcctcgtcgccgtcgcgcgcccacgctcgcgctcccacccgcttcgcggtcgcagcatccgcgcgcgccgcacggttccgccccgcgcgcgccatggccgcctccgacgacccccgcggcgggaggtccgtcgccgtcgtcggcgccggcgtcaa

SEQ ID NO: 34 - DNA, 329 bp, Artificial Sequence
  Sequencing result of new PPO2 gene from rice callus sample QY1387/818-70#
tttaggatttcttgcaattccattttggcacttttcttctgacctatttctctggctgctgcttcctgataatcgaccagttctctagtcttgctccctgcactcctccctccctatccaagagccccaaatcagatgctctctcctgccaccaccttctcctcctcctcctcctcctcgtcgccgtcgcgcgcccacgctcgcgctcccacccgcttcgcggtcgcagcatccgcgcgcgccgcacggttccgccccgcgcgcgccatggccgcctccgacgacccccgcggcgggaggtccgtcgccgtcgtcggcgccggcgtcag

SEQ ID NO: 35 - DNA, 307 bp, Artificial Sequence
  Sequencing result of new PPO2 gene from rice callus sample QY1387/818-82#
tttcttgcaattccattttggcacttttcttctgacctatttctctggctgctgcttcctgataatcgaccagttctctagtcttgctccctgcactcctgccccaaatcagatgctctctcctgccaccaccttctcctcctcctcctcctcctcgtcgccgtcgcgcgcccacgctcgcgctcccacccgcttcgcggtcgcagcatccgcgcgcgccgcacggttccgccccgcgcgcgccatggccgcctccgacgacccccgcggcgggaggtccgtcgccgtcgtcggcgccggcgtcagg

SEQ ID NO: 36 - DNA, 348 bp, Artificial Sequence
  Sequencing result of new PPO2 gene from rice callus sample QY1387/818-145#
tttcggcttctccatcctagtagtttaggatttcttgcaattccattttggcacttttcttctgacctatttctctggctgctgcttcctgataatcgaccagttctctagtcttgctccctgcactcctccctcctctatccaagagccccaaatcagatgctctctcctgccaccaccttctcctcctcctcctcctcctcgtcgccgtcgcgcgcccacgctcgcgctcccacccgcttcgcggtcgcagcatccgcgcgcgccgcacggttccgccccgcgcgcgccatggccgcctccgacgacccccgcggcgggaggtccgtcgccgtcgtcggcgccggc

SEQ ID NO: 37 - DNA, 333 bp, Artificial Sequence
  Sequencing result of new PPO2 gene from rice callus sample QY2611/818-3#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagagagcatctgatttggggctcttggataggactaaactaactagtagatagatagagctcagcggcggatgagtgaaacgggacgagcggcggcgtgactggcgcgtataaaaggcgcaacgtgcgtcacgcccgaccggacaccg

SEQ ID NO: 38 - DNA, 337 bp, Artificial Sequence
  Sequencing result of new PPO2 gene from rice callus sample QY2611/818-10#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagagagcatctgatttggggctcttggatagagacgactaaactaactagtagatagatagagctcagcggcggatgagtgaaacgggacgagcggcggcgtgactggcgcgtataaaaggcgcaacgtgcgtcacgcccgaccggacaccg

SEQ ID NO: 39 - DNA, 311 bp, Artificial Sequence
  Sequencing result of new PPO2 gene from rice callus sample QY2611/818-55#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagagagcatctgatttggggctcttggatagatagagctcagcggcggatgagtgaaacgggacgagcggcggcgtgactggcgcgtataaaaggcgcaacgtgcggcacgcccgaccggacaccg

SEQ ID NO: 40 - DNA, 307 bp, Artificial Sequence
  Sequencing result of new PPO2 gene from rice callus sample QY2611/818-67#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagagagcatctgatttggggctcttggatagagctcagcggcggatgagtgaaacgggacgagcggcggcgtgactggcgcgtataaaaggcgcaacgtgcgtcacgcccgaccggacaccg

SEQ ID NO: 41 - DNA, 748 bp, Artificial Sequence
  Sequencing result of new PPO2 gene from rice callus sample QY2611/818-90#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagagagcatctgatttggggctcttggatagaggtcctcgcgattgagcttcaccagcagctcctccgtgccgtccatcttctccagaatcggcttgatgaacttgtagaactcctcctgagaggccccgccgtcaatgtacccagcgtagccgttcttcgactgatcgaagaagatctccttgtacttctcggggagctgctgcctgaccagcgccttcaggagggtcagatcctgatggtgctcgtcgtagcgcttgatcatggaggctgagagcggagccttcgtaatctcggtgttcaccctgagaatatcagacaggaggatggcgtccgacagattcttggcagcgaggaacaggtccgcgtactgatcgccgatctgggccaggaggttatccaggtcatcgtcgtatgtgtccttggagagctgcagcttggcgtcctcagcgccgactaaactaactagtagatagatagagctcagcggcggatgagtgaaacgggacgagcggcggcgtgactggcgcgtataaaaggcgcaacgtgcgtcacgcccgaccg

SEQ ID NO: 42 - DNA, 335 bp, Artificial Sequence
  Sequencing result of new PPO2 gene from rice callus sample QY2611/818-108#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagagagcatctgatttggggctcttggatagacgactaaactaactagtagatagatagagctcagcggcggatgagtgaaacgggacgagcggcggcgtgactggcgcgtataaaaggcgcaacgtgcgtcacgcccgaccggacaccg

SEQ ID NO: 43 - DNA, 315 bp, Artificial Sequence
  Sequencing result of new PPO2 gene from rice callus sample QY2611/818-121#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagagagcatctgtgtataaaaggcacagtagatagatagagctcagcggcggatgagtgaaacgggacgagcggcggcgtgactggcgcgtataaaaggcgcaacgtgcgtcacgcccgaccggacaccg

SEQ ID NO: 44 - DNA, 285 bp, Artificial Sequence
  Sequencing result of new PPO2 gene from rice callus sample QY2612/818-5#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagagagcatctgatttggggctcttggatagggttggttggtgtcgtttcggtttcggcgaatgtaacggtcacgtaggagtatgtgtagtgtagcaagg

SEQ ID NO: 45 - DNA, 282 bp, Artificial Sequence
  Sequencing result of new PPO2 gene from rice callus sample QY2612/818-34#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagagagcatctgatttggggctcttggaggttggttggtgtcgtttcggtttcggcgaatgtaacggtcacgtaggagtatgtgtagtgtagcaagg

SEQ ID NO: 46 - DNA, 286 bp, Artificial Sequence
  Sequencing result of new PPO2 gene from rice callus sample QY2612/818-66#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagagagcatctgatttggggctcttggatagtggttggttggtgtcgtttcggtttcggcgaatgtaacggtcacgtaggagtatgtgtagtgtagcaagg

SEQ ID NO: 47 - DNA, 254 bp, Artificial Sequence
  Sequencing result of new PPO2 gene from rice callus sample QY2612/818-377#
ctgacgccggcgccgacgacggcgacggacctcccgccgcgggggtcgtcggaggcggccatggcgcgcgcggggcggaaccgtgcggcgcgcgcggatgctgcgaccgcgaagcgggtgggagcgcgagcgtgggcgcgcgacggcgacgaggaggaggaggaggaggagaaggtggtggcaggagaaggttggtgtcgtttcggtttcggcgaatgtaacggtcacgtaggagtatgtgtagtgtagcaagg

SEQ ID NO: 48 - DNA, 494 bp, Artificial Sequence
  Sequencing result of new PPO2 gene resulting from maize protoplast sample transformed by pQY1340 vector
tcatcacaatgatgctcttcattctacctcttgtattttattagatatatgttgatagtaacatctattttttcttgttgcagtgcgcgaagaagccatcctgatatcccttttggcttcctcattctagtagtttaggatttcttttctgacactttgattctgaccaatctctctggcctgctcgacaacaagcaaatccccatccagttccaaaccctaactcaaatgctcgctttgactgcctcagcctcatccgcttcgtcccatccttatcgccacgcctccgcgcacactcgtcgcccccgcctacgtgcggtcctcgcgatggcgggctccgacgacccccgtgcagcgcccgccagatcggtcgccgtcgtcggcgccggggtcaggtgagcgagcgctctttcactctccgagcctcgggtgcgagggttttggtacgtgtttgtgccttcgaggcgtttggtgaaatggttgtgtggtgcttt

SEQ ID NO: 49 - DNA, 536 bp, Artificial Sequence
  Sequencing result of new PPO2 gene resulting from maize protoplast sample transformed by pQY1341 vector
tcatcacaatgatgctcttcattctacctcttgtattttattagatatatgttgatagtaacatctattttttcttgttgcagtgcgcgaagaagccatcctgatatcccttttggcttcctcattctagtagtttaggatttcttttctgacactttgattctgaccaatctctctggcctgctgcttcctgataatcgaccagttccccagtcttgctccttgcactcctcccccaaaacaagcaaatccccatccagttccaaaccctaactcaaatgctcgctttgactgcctcagcctcatccgcttcgtcccatccttatcgccacgcctccgcgcacactcgtcgcccccgcctacgtgcggtcctcgcgatggcgggctccgacgacccccgtgcagcgcccgccagatcggtcgccgtcgtcggcgccggggtcaggtgagcgagcgctctttcactctccgagcctcgggtgcgagggttttggtacgtgtttgtgccttcgaggcgtttggtgaaatggttgtgt

SEQ ID NO: 50 - DNA, 891 bp, Artificial Sequence
  Sequencing result of new PPO2 gene resulting from wheat protoplast sample transformed by pQY2626 vector
atgatgtccggcatgttatgttaaccatcgaataattgatattaacaagaaaataactgcatagaaacgttttcatagttatctagccatgaaagtttgctagaacctgctgtctaacccctcttttctcgtttcgcagttagtgaatgtgctaatggagtcaaagggtggcaaaaagtctagcagtagtagttccctgatgtacgaagcacccctcggctacagcattgaagacgttcgacctgctggaggcgccaagaagttctctgctgcatactcgaacgtaaggatcacggacgaaacatcagagtctcacttctgttcaaacattttatctccagatttgttgctaaccatgttactccgcggctccgatcggcgacagcgacggagaaaaacgacaagcggctcctctccccatccaaaccccgaatcaaatgctcacttccgccaccgccccctcctcctcctgctcgtcccacgctcccacccgcttcgcctccgcgcgcgggcgccgcctccgccccgtgctcgccatggccgcctccgacgacccccgcgccagatcggtcgccgtcgtcggcgccggcgtcaggtgagtccacacgatttcgcgccgaattctcgggtgggggttttgcgctcgcgcacgtcaggtgtttgacggaatggtgactccgtgcagtgggctcgtggcggcgtacaggctgaggaagagcggcgtgcgggtcaccgtgttcgaggcggaagaccgcgcgggagggaagatacggaccaactccgacggcggattcctctgagacgactgagacaacactatggatgagctcatttctgtttgtgaataacttgtggcgcttggtgtagcgctgtctctgtttggcccacgga

SEQ ID NO: 51 - DNA, 444 bp, Artificial Sequence
  misc_feature (439)..(441): n is a, c, g, or t; Sequencing result of new PPO2 gene resulting from wheat protoplast sample transformed by pQY2627 vector
gttcgttgttcgattttacaatatgatgtccggcatgttatgttaaccatcgaataattgatattaacaagaaaataactgcatagaaacgttttcatagttatctagccatgaaagtttgctagaacctgctgtctaacccctcttttctcgtttcgcagttagtgaatgtgctaatggagtcaaagggtggcaaaaagtctagcagtagtagttccctgatgtacgaagcacccctcggctacagcattgaagacgttcgacctgctggaggcgccaagaagttctctgctgcatactcgaacgtaaggatcacggacgaaacatcagagtctcacttctgttcaaacattttatctccagatttgttgctaaccatgtgaatttgtttcctgcagtgcgcgaagaagccatcctgatatcgtttttggcatccccnnnacg

SEQ ID NO: 52 - DNA, 874 bp, Artificial Sequence
  Sequencing result of new PPO2 gene resulting from wheat protoplast sample transformed by pQY2630 vector
aattgtgtaaattaattttaaaatatgcatctgctcccgtagagtcacaaagtttcaaatggtacacatgttcgtggttgaattttattagccatcaatataattttatagaaacgtttccttagttttgctagaatctgttgtctaacccctcttatcattttgcagttagtgaatgtgctaatggagtcaaagggtggcaaaaagtctagcagtagtagttccctgatgtacgaagctcccctcggctacagcattgaagacgttcgacctgctggaggcgccaagaagttctctgctgcatactcgaacgtaaggacgagacccaagagccttcacttctgttcagacattttatctccagatttgttgctaaccatgtccgcggctccgatcggcgacagcgacggagaaaagcgacgagcgactcctctccccatccaaaccccgaatcaaatgctcacttccgccaccgctcccccctcctcttcctcctgctcgtcccacgctcccacccgcttcgcctccgcgcgcgggcgacgcctccgccccgtgctcgccatggccgcctccgacgacccccgcgccaggtcggtcgctgtcgtcggcgccggcgtcaggtgaggccacacgatttcgcgccgaattctcgggtggttttgcgctcgcgcacgtgaagtgtttgacggaatggttggtccgtgcagtggtctcgtggcggcgtacaggctgaggaagagcggcgtgcgggtcaccgtgttcgaggcggaagaccgcgcgggagggaagatacggaccaactccgacggcggattcctctgggacgaaggagccaacaccatggtgagcccatttctgtctgttaactaacttg

SEQ ID NO: 53 - DNA, 894 bp, Artificial Sequence
  Sequencing result of new PPO2 gene resulting from wheat protoplast sample transformed by pQY2631 vector
agacgcgacgcacagcaccacaagttagttaacagacagaaatgggctcaccatggtgttggctccttcgtcccagaggaatccgccgtcggagttggtccgtatcttccctcccgcgcggtcttccgcctcgaacacggtgacccgcacgccgctcttcctcagcctgtacgccgccacgagaccactgcacggaccaaccattccgtcaaacacttcacgtgcgcgagcgcaaaaccacccgagaattcggcgcgaaatcgtgtggcctcacctgacgccggcgccgacgacagcgaccgacctggcgcgggggtcgtcggaggcggccatggcgagcacggggcggaggcgtcgcccgcgcgcggaggcgaagcgggtgggagcgtgggacgagcaggaggaagaggaggggggagcggtggcggaagtgagcatttgattcggggtttggatggggagaggagtcgctcgtcgcttttctccgtcgctgtcgccgatcggagccgcggagaagggtcttgggtctcctccttaccttccaatatgcaacaaaaaacttcttggcgcctcccacagggccaacgtcttcaatggtggaaccgaagggaacttcctacccttagggaactactactggtaaaatttttgccaccctttgactccattaacaccttcactaactggaaaatgaaaaaaaggggtagaccacagaatctaacaaaactaaggaaacgtttctataaaattatattgatgggtaataaaattccaccaccaacatgtggaccatttgaaactttgtgactctacgggaacagaaggatattttaaaattaatttacacaatttaattcactaatctcatggacacaaaaaaaaaaaaaaaaaaaa

SEQ ID NO: 54 - DNA, 860 bp, Artificial Sequence
  Sequencing result of new PPO2 gene resulting from wheat protoplast sample transformed by pQY2634 vector
aagggggcaaaaagtatagcagtagtagttccgagatgtgcgaagctcccctcggctacagcatagaagacgttcgacgtgctggaggcgccaagaagttctctgctgcatactcgaacgtaaggacaattctcacacacgagtctcatttgtgtttagacattttatctccagatttgttgctacccatgtgacatttttttatctcccgcagtgcgagaaaaacccatcgagatatcgttttgggcatccctttcccgtagtttaggatttttacgcaatttttttgtgtcttttttctcccaccaatctctctggcctgctacctactactccgcggcgccgatcggcgacagcgacggagaaaggcgacgagcggctcctctccccatccaaaccccgaatcaaatgctcacttccgccaccgctccctcctcctcgtcgtcctgcgctcccacccgcttcgcctcctcgtgccgcccccgtcgctccgcctccgcgcgcgggcgccgccccgtgctcgccatggccgcctccgacgacccccgcgccaggtcggtcgccgtcgtcggcgccggcgtcaggtgaggccacaagatttcgcgccgaattctcgggtggttttgcgctcgcgcacgtcaggtgtttgacggaatgatgggtctgtgcagtgggctcgtggcggcgtacaggctgaggaagagcggcgtgcgggtcaccgtgttcgaggcggaggaccgcgcgggagggaagatacggaccaactccgacggcggattcctctgggacgaaggagccaacaccatggtgagcccatttctgtttgttaactaacttgtggcgttgtgcatcgctgcctc

SEQ ID NO: 55 - DNA, 707 bp, Artificial Sequence
  Sequencing result of new PPO2 gene resulting from wheat protoplast sample transformed by pQY2635 vector
aacaagagtctctcttctttttagaccttttatcctcagtgttggttatcaacatgtcacttttttttttcttccgcagggcaagaagacatccttctaatattgtttccttcatcccccttcaggagtttaagtttttgaagcaattttattctgactcttttctcccatacctactactccgcggcgccgatcggcgacagcgacggagaaaggcgacgagcggctcctctccccatccaaaccccgaatcaaatgctcacttccgccaccgctccctcctcctcgtcgtcctgcgctcccacccgcttcgcctcctcgtgccgcccccgtcgctccgcctccgcgcgcgggcgccgccccgtgctcgccatggccgcctccgacgacccccgcgccaggtcggtcgccgtcgtcggcgccggcgtcaggtgaggccacaagatttcgcgccgaattctcgggtggttttgcgctcgcgcacgtcaggtgtttgacggaatgatgggtctgtgcagtgggctcgtggcggcgtacaggctgaggaagagcggcgtgcgggtcaccgtgttcgaggcggaggaccgcgcgggagggaagatacggaccaactccgacggcggattcctctgggacgaaggagccaacaccatggtgagcccatttctgtttgttaactaacttgtggcgttgtgcatcgctgcctc

SEQ ID NO: 56 - DNA, 871 bp, Artificial Sequence
  Sequencing result of new PPO2 gene resulting from wheat protoplast sample transformed by pQY2636 vector
ctcggctacagcattgaagacgttcgacctgctggaggcgccaagaagttctctgctgcatactcgaacgtaaggacaattctcaaacacgagtctcacttctgtttagacattttatctccagatttgttgctaaccatgtgacattttttcatctcctgcagtgcgcgaagaagccatcctgatatcgtttttggcatccccttcccgtagtttaggatttttatgcaattttattctgactcttttctcccaccaatctctctggcctgcagcaggcgtcgtggcataaccagggcgccgcctccggtttcgacccactgactagtctacccacccctgctacctactactccgcggcgccgatcggcgacagcgacggagaaaggcgacgagcggctcctctccccatccaaaccccgaatcaaatgctcacttccgccaccgctccctcctcctcgtcgtcctgcgctcccacccgcttcgcctcctcgtgccgcccccgtcgctccgcctccgcgcgcgggcgccgccccgtgctcgccatggccgcctccgacgacccccgcgccaggtcggtcgccgtcgtcggcgccggcgtcaggtgaggccacaagatttcgcgccgaattctcgggtggttttgcgctcgcgcacgtcaggtgtttgacggaatgatgggtctgtgcagtgggctcgtggcggcgtacaggctgaggaagagcggcgtgcgggtcaccgtgttcgaggcggaggaccgcgcgggagggaagatacggaccaactccgacggcggattcctctgggacgaaggagccaacaccatggtgagcccatttctgtttgttaactaacttgtggcgttgtgcat

SEQ ID NO: 57 - DNA, 593 bp, Artificial Sequence
  Sequencing result of new C9.PPO2 gene from T0 generation of oilseed rape event QY2533/ W-7
tttatatattagctgagatacaggtagagaaaaatactcatcgaagggttgtaaggccacttattcgattaacttagaaaattgccacgttatattctctattcttgatttatcaaaaagttttattattcttgatacgtgccacataaatacataaactagtgttcgaaaatcaagaagtccaaagtggcaatcatatagatttatcaaacaagttaaatattactaagttatatcaacaagttaacggagctaacaataaataataatatgcctatatctgataaaggcgcgagtccaggctttacgatacaaaaatgggccctgttaaagcccaataagtactttatttcgtaaattacatttctagaatcaacggaacccaacagaaacgcgtctggactcgacgttgtgccttatccgtagctctcatcttcttcgtcgcctctgttctcttcatctggtcagacgttttgacgctgaagtgtgttgcttttgcagatagcaatggcgtctaacgcagcagcagatcatgataaacgttagagaacttctatttcagctgattgtgtagatttttcagtttttttttt

SEQ ID NO: 58 - DNA, 563 bp, Artificial Sequence
  Sequencing result of new C9.PPO2 gene from T0 generation of oilseed rape event QY2533/ W-36
gaaaaatactcatcgaagggttgtaaggccacttattcgattaacttagaaaattgccacgttatattctctattcttgatttatcaaaaagttttattattcttgatacgtgccacataaatacataaactagtgttcgaaaatcaagaagtccaaagtggcaatcatatagatttatcaaacaagttaaatattactaagttatatcaacaagttaacggagctaacaataaataataatatgcctatatctgataaaggcgcgagtccaggctttacgatacaaaaatgggccctgttaaagcccaataagtactttatttcgtaaattacatttctagaatcaacggaacccaacagaaacgcgtctggactcgacgttgtgccttatccgtagctctcatcttcttcgtcgcctcttctcttcatctggtcagacgttttgacgctgaagtgtgttgcttttgcagatagcaatggcgtctaacgcagcagcagatcatgataaacgttagagaacttctatttcagctgattgtgtagatttttcagtttttttttt

SEQ ID NO: 59 - DNA, 587 bp, Artificial Sequence
  Sequencing result of new C9.PPO2 gene from T0 generation of oilseed rape event QY2533/ W-42
tagctgagatacaggtagagaaaaatactcatcgaagggttgtaaggccacttattcgattaacttagaaaattgccacgttatattctctattcttgatttatcaaaaagttttattattcttgatacgtgccacataaatacataaactagtgttcgaaaatcaagaagtccaaagtggcaatcatatagatttatcaaacaagttaaatattactaagttatatcaacaagttaacggagctaacaataaataataatatgcctatatctgataaaggcgcgagtccaggctttacgatacaaaaatgggccctgttaaagcccaataagtactttatttcgtaaattacatttctagaatcaacggaacccaacagaaacgcgtctggactcgacgttgtgccttatccgtagctctcatcttcttcgtcgcctctgtttctcttcatctggtcagacgttttgacgctgaagtgtgttgcttttgcagatagcaatggcgtctaacgcagcagcagatcatgataaacgttagagaacttctatttcagctgattgtgtagatttttcagtttttttttttt

SEQ ID NO: 60 - DNA, 670 bp, Artificial Sequence
  Sequencing result of new C9.PPO2 gene from T0 generation of oilseed rape event QY2535/ W-32
atttatatattagctgagatacaggtagagaaaaatactcatcgaagggttgtaaggccacttattcgattaacttagaaaattgccacgttatattctctattcttgatttatcaaaaagttttattattcttgatacgtgccacataaatacataaactagtgttcgaaaatcaagaagtccaaagtggcaatcatatagatttatcaaacaagttaaatattactaagttatatcaacaagttaacggagctaacaataaataataatatgcctatatctgataaaggcgcgagtccaggctttacgatacaaaaatgggccctgttaaagcccaataagtactttatttcgtaaattacatttctagaatcaacggaacccaaaacagatgagagctacggataaggcataacttaagatgatagtggactaatgggcctgagaattgaaacgacaccctttgttgtcgtttcaggagcaggtatttgttttataacttttatttattcatctccttcctgctttctcttcatctggtcagacgttttgacgctgaagtgtgttgcttttgcagatagcaatggcgtctaacgcagcagcagatcatgataaacgttagagaacttctatttcagctgattgtgtagatttttcagtttttttttt

SEQ ID NO: 61 - DNA, 570 bp, Artificial Sequence
  Sequencing result of new C9.PPO2 gene from T0 generation of oilseed rape event QY2536/ W-124
gaaaaatactcatcgaagggttgtaaggccacttattcgattaacttagaaaattgccacgttatattctctattcttgatttatcaaaaagttttattattcttgatacgtgccacataaatacataaactagtgttcgaaaatcaagaagtccaaagtggcaatcatatagatttatcaaacaagttaaatattactaagttatatcaacaagttaacggagctaacaataaataataatatgcctatatctgataaaggcgcgagtccaggctttacgatacaaaaatgggccctgttaaagcccaataagtactttatttcgtaaattacatttctagaatcaacggaacccaacagaaacgcgtctggactcgacgttgtgccttatccgtagctctcatcttcttcgtcgcctctgagttgtcgtttcaggagcaggtatttgttttataacttttatttattcatctccttcctgctttctcttcatctggtcagacgttttgacgctgaagtgtgttgcttttgcagatagcaatggcgtctaacgcagcagcagatcatga

SEQ ID NO: 62 - DNA, 627 bp, Artificial Sequence
  Sequencing result of new WAK1 gene from T0 generation of rice event QY1085/ 818-57
taaacgtcgtcttcgcgagggtccaaaaggcagtatttaggtgccgtacagtgactgcacgactgtgggggcctcgctgtttcgatcgtttcacacgctggagaagaagaaggaaacgacgtggcgccgatcgtcgccgtcgatcggtgggaggaaggtgaagccgtgcccgagctttcgtcgtctctcgttggaaagctactgttttgacccgttcacaccactacgcttccagctataggcagtatgtacggtgccacgcacggctctcgtcctttatataaaccaactgcctcgccattgccaccagcagcagcagctcgccatcctgtagcagctagctgaaccacgagtggcaagatgcacccgaccttgttgtgcttgccactcttggcctccttgctcctcctgtgccatcccgcgcgcgccgagtgcgagccggcgacatgcggcaacctcaccgtcaggtacccattctgggtaggcggccccgacctcaaccggtcgtcgtccgctatctcctgtggccacccggccttcgaggtgtggtgtaccggcggcgtggcgtcgttgaggggctcgcaaatcctcgtcctcagcatcgactacaacagcagctcattcgtc

SEQ ID NO: 63 - DNA, 751 bp, Artificial Sequence
  Sequencing result of new WAK1 gene from T0 generation of rice event QY1089/ 818-595
ctgaggacgaggatttgcgagcccctcaacgacgccacgccgccggtacaccacacctcgaaggccgggtggccacaggagatagcggacgacgaccggttgaggtcggggccgcctacccagaatgggtacctgacggtgaggttgccgcatgtcgccggctcgcactcggcgcgcgcgggatggcacaggaggagcaaggaggccaagagtggcaagcacaacaaggtcgggtgcatcttgccactcgtggttcagctagctgctacagctatttcctctacttggtactagtagatggctttacgctttgtgtttgtggttatggccaaagcccaggagagcatctgtatatataggcgttcagatctgaacaacatattagcatgttgccaaatgccattctttcatgcttgatatcgaatatattttttggggatgcatgttaggcgtgcctttgactgacgcggctgctagagtgtgaggcgttgaacacattttgtattcttccatgtcacactcacgctcctatcgtcctcgtcagcggtccaaaatggacgaaattgacaccaccagcaccaacaaaaatcgttcagttgtacagtggtaagctttgcaaatctttaagggaattaataatgatttccccatctcatcacacaaacgaattacctgttttatattattatgtataaatagaactcatgggggttcaaaacactattttatcaactagtagtcctattttttt

SEQ ID NO: 64 - DNA, 629 bp, Artificial Sequence
  Sequencing result of new WAK1 gene from T0 generation of rice event QY1085/ 818-107
ttaaacgtcgtcttcgcgagggtccaaaaggcagtatttaggtgccgtacagtgactgcacgactgtgggggcctcgctgtttcgatcgtttcacacgctggagaagaagaaggaaacgacgtggcgccgatcgtcgccgtcgatcggtgggaggaaggtgaagccgtgcccgagctttcgtcgtctctcgttggaaagctactgttttgacccgttcacaccactacgcttccagctataggcagtatgtacggtgccacgcacggctctcgtcctttatataaaccaactgcctcgccattgccaccagcagcagcagctcgccatccatgtagcagctagctgaaccacgagtggcaagatgcacccgaccttgttgtgcttgccactcttggcctccttgctcctcctgtgccatcccgcgcgcgccgagtgcgagccggcgacatgcggcaacctcaccgtcaggtacccattctgggtaggcggccccgacctcaaccggtcgtcgtccgctatctcctgtggccacccggccttcgaggtgtggtgtaccggcggcgtggcgtcgttgaggggctcgcaaatcctcgtcctcagcatcgactacaacagcagctcattcgtc

SEQ ID NO: 65 - DNA, 628 bp, Artificial Sequence
  Sequencing result of new WAK1 gene from T0 generation of rice event QY1085/ 818-167
attaaacgtcgtcttcgcgagggtccaaaaggcagtatttaggtgccgtacagtgactgcacgactgtgggggcctcgctgtttcgatcgtttcacacgctggagaagaagaaggaaacgacgtggcgccgatcgtcgccgtcgatcggtgggaggaaggtgaagccgtgcccgagctttcgtcgtctctcgttggaaagctactgttttgacccgttcacaccactacgcttccagctataggcagtatgtacggtgccacgcacggctctcgtcctttatataaaccaactgcctcgccattgccaccagcagcagcagctcgccatccaatgtagcagctagctgaaccacgagtggcaagatgcacccgaccttgttgtgcttgccactcttggcctccttgctcctcctgtgccatcccgcgcgcgccgagtgcgagccggcgacatgcggcaacctcaccgtcaggtacccattctgggtaggcggccccgacctcaaccggtcgtcgtccgctatctcctgtggccacccggccttcgaggtgtggtgtaccggcggcgtggcgtcgttgaggggctcgcaaatcctcgtcctcagcatcgactacaacagcagctcattc

SEQ ID NO: 66 - DNA, 620 bp, Artificial Sequence
  Sequencing result of new WAK1 gene from T0 generation of rice event QY1085/ 818-23
cgtcgtcttcgcgagggtccaaaaggcagtatttaggtgccgtacagtgactgcacgactgtgggggcctcgctgtttcgatcgtttcacacgctggagaagaagaaggaaacgacgtggcgccgatcgtcgccgtcgatcggtgggaggaaggtgaagccgtgcccgagctttcgtcgtctctcgttggaaagctactgttttgacccgttcacaccactacgcttccagctataggcagtatgtacggtgccacgcacggctctcgtcctttatataaaccaactgcctcgccattgccaccagcagcagcagctcgccatccaatagcagctagctgaaccacgagtggcaagatgcacccgaccttgttgtgcttgccactcttggcctccttgctcctcctgtgccatcccgcgcgcgccgagtgcgagccggcgacatgcggcaacctcaccgtcaggtacccattctgggtaggcggccccgacctcaaccggtcgtcgtccgctatctcctgtggccacccggccttcgaggtgtggtgtaccggcggcgtggcgtcgttgaggggctcgcaaatcctcgtcctcagcatcgactacaacagcagctcattc

SEQ ID NO: 67 - DNA, 734 bp, Artificial Sequence
  Sequencing result of new WAK1 gene from T0 generation of rice event QY1089/ 818-321
ttgcgagcccctcaacgacgccacgccgccggtacaccacacctcgaaggccgggtggccacaggagatagcggacgacgaccggttgaggtcggggccgcctacccagaatgggtacctgacggtgaggttgccgcatgtcgccggctcgcactcggcgcgcgcgggatggcacaggaggagcaaggaggccaagagtggcaagcacaacaaggtcgggtgcatcttgccactcgtggttcagctagctgctacaatttcctctacttggtactagtagatggctttacgctttgtgtttgtggttatggccaaagcccaggagagcatctgtatatataggcgttcagatctgaacaacatattagcatgttgccaaatgccattctttcatgcttgatatcgaatatattttttggggatgcatgttaggcgtgcctttgactgacgcggctgctagagtgtgaggcgttgaacacattttgtattcttccatgtcacactcacgctcctatcgtcctcgtcagcggtccaaaatggacgaaattgacaccaccagcaccaacaaaaatcgttcagttgtacagtggtaagctttgcaaatctttaagggaattaataatgatttccccatctcatcacacaaacgaattacctgttttatattattatgtataaatagaactcatgggggttcaaaacactattttatcaactagtagtcctattttttt

SEQ ID NO: 68 - DNA, 716 bp, Artificial Sequence
  Sequencing result of new WAK1 gene from T0 generation of rice event QY1089/ 818-312
ttgcgagcccctcaacgacgccacgccgccggtacaccacacctcgaaggccgggtggccacaggagatagcggacgacgaccggttgaggtcggggccgcctacccagaatgggtacctgacggtgaggttgccgcatgtcgccggctcgcactcggcgcgcgcgggatggcacaggaggagcaaggaggccaagagtggcaagcacaacaaggtcgggtgcatcttgccactcgtggttcagctagcttggtactagtagatggctttacgctttgtgtttgtggttatggccaaagcccaggagagcatctgtatatataggcgttcagatctgaacaacatattagcatgttgccaaatgccattctttcatgcttgatatcgaatatattttttggggatgcatgttaggcgtgcctttgactgacgcggctgctagagtgtgaggcgttgaacacattttgtattcttccatgtcacactcacgctcctatcgtcctcgtcagcggtccaaaatggacgaaattgacaccaccagcaccaacaaaaatcgttcagttgtacagtggtaagctttgcaaatctttaagggaattaataatgatttccccatctcatcacacaaacgaattacctgttttatattattatgtataaatagaactcatgggggttcaaaacactattttatcaactagtagtcctattttttt

SEQ ID NO: 69 - DNA, 671 bp, Artificial Sequence
  Sequencing result of new CNGC gene from T0 generation of rice event QY1090/ 818-192
ctgcaaaaatgttatctctgaatgtgtattatcttttgtgcaaatattgggtggttccaatcagaaagggtaacaattagaccagaattgcatattgacaacatatcgcctcctgtgtccacctgttcgcaactgcggatcaagctactacattaaaaggggcaaactaaatctgccgtataaattgaaactttcctccttacatatgcttgaaagaaccgaaaagaattcctcttctgcatcaagagacgaaaaggaagctacaggattaaagaatcacaagctggcagcacgcacctgttggtagtcctcctctgcatctccacctcatcctggactcgggacccaaacatggcggctgctccccttccccccgtcgaaggaaggggataagaggcgacgattttggggggaggagaaggttggttgggttgggttcatgaatcatgatatttccttcttctcctttcgctttccttatcccaagtccatgggcccaaatcacggcccatataggagtacgtgcatagccatctgggccgtacatattttaaaataaatcgacaaaagtgaaattaagctttgtttatataataatgttgtgacttgcctgtaattagaatagggattaaattttatactcttaccactggatcactgattagcatgac

SEQ ID NO: 70 - DNA, 673 bp, Artificial Sequence
  Sequencing result of new CNGC gene from T0 generation of rice event QY1090/ 818-554
taaactgcaaaaatgttatctctgaatgtgtattatcttttgtgcaaatattgggtggttccaatcagaaagggtaacaattagaccagaattgcatattgacaacatatcgcctcctgtgtccacctgttcgcaactgcggatcaagctactacattaaaaggggcaaactaaatctgccgtataaattgaaactttcctccttacatatgcttgaaagaaccgaaaagaattcctcttctgcatcaagagacgaaaaggaagctacaggattaaagaatcacaagctggcagcacgcacctgttggtagtcctcctctgcatctccacctcatcctggactcgggacccaaacatggcggctgctccccttccccccgcgaaggaaggggataagaggcgacgattttggggggaggagaaggttggttgggttgggttcatgaatcatgatatttccttcttctcctttcgctttccttatcccaagtccatgggcccaaatcacggcccatataggagtacgtgcatagccatctgggccgtacatattttaaaataaatcgacaaaagtgaaattaagctttgtttatataataatgttgtgacttgcctgtaattagaatagggattaaattttatactcttaccactggatcactgattagcatga

SEQ ID NO: 71 - DNA, 683 bp, Artificial Sequence
  Sequencing result of new CNGC gene from T0 generation of rice event QY1090/ 818-541
aataaactgcaaaaatgttatctctgaatgtgtattatcttttgtgcaaatattgggtggttccaatcagaaagggtaacaattagaccagaattgcatattgacaacatatcgcctcctgtgtccacctgttcgcaactgcggatcaagctactacattaaaaggggcaaactaaatctgccgtataaattgaaactttcctccttacatatgcttgaaagaaccgaaaagaattcctcttctgcatcaagagacgaaaaggaagctacaggattaaagaatcacaagctggcagcacgcacctgttggtagtcctcctctgcatctccacctcatcctggactcgggacccaaacatggcggctgctcaagcttggatcctacaaatgccatcattgcgataagaggcgacgattttggggggaggagaaggttggttgggttgggttcatgaatcatgatatttccttcttctcctttcgctttccttatcccaagtccatgggcccaaatcacggcccatataggagtacgtgcatagccatctgggccgtacatattttaaaataaatcgacaaaagtgaaattaagctttgtttatataataatgttgtgacttgcctgtaattagaatagggattaaattttatactcttaccactggatcactgattagcatgac

SEQ ID NO: 72 - DNA, 794 bp, Artificial Sequence
  Sequencing result of new CNGC gene from T0 generation of rice event QY1094/ 818-202
gcggcggctgctgggctctcgcccaggcttgttgttacgccggggaggagaagccacagccatggacggcggcggatgtgctgcgccgcggcggcggcggcgcagggggagaggaagacgacggtgaggagcaaggagggagacgcggtggagttgtgccgggtggtgaacggcatgtggcaggtgagtggcccgtcgtggggccgctacgagacgcccgccgccgtggacgccatgctccggtacgccgacggcggcctctccaccttcgacatggctgacatttgtaagcctctcctcccgccgcattctggatacctcgcaggaaactgacacgtggggcccactcgtagtgacacgtactgtagctgcgtgaaatttaccccgtccgtgccgcgaggggggaaggggagcagccgccatgtttgggtcccgagtccaggatgaggtggagatgcagaggaggactaccaacaggtgcgtgctgccagcttgtgattctttaatcctgtagcttccttttcgtctcttgatgcagaagaggaattcttttcggttctttcaagcatatgtaaggaggaaagtttcaatttatacggcagatttagtttgccccttttaatgtagtagcttgatccgcagttgcgaacaggtggacacaggaggcgatatgttgtcaatatgcaattctggtctaattgttaccctttctgattggaaccacccaatatttgcacaaaagataatacacattcagagataacatttttgcagtttatttttccttactgggt